SEQUENCE LISTING (29 sequences)

SEQ ID NO: 1 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 2 - protein, 710 aa, Mus musculus
MNQELLSVGSKRRRTGGSLRGNASSSQVDEGQMNRVVEEDPQQQARHQEEEHTARNGELVGANPRPGDQNDTQQGQVEENNNRFISVDEDSSGNQEEQEEDEEHAGEQEEEEEEEEEEEEMDQESDDFDPSDDSSREDEHTHNSNVTNCSSVSDLPAHQLSSPFYTKTTKMKRKLDHGSEVRSFSLGKKPCKVSDYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKIIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPSKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 3 - DNA, 213763 bp, Homo sapiens
ccttccgcagctgccgcttcagtccgaaggaggaagggaaccaacccactttctcggcgccgcggctcttttctaaaagtgtgagtggccccgggagagggaattgagggggagaaagtgggaggatgccggcgcttcctcctggagttttgctgatgttctccagtagtgttgggagtctatcaatgagggagtctagctttattcacggaggggggaaggtgttagggaggcctgtattttgtaactttttagctctttgtacgagtaatgcaagtatgcaggtttctgtcggaggagtcttttctccaaagtgaatactcgcacctttctggccagcagttggcaaagggaaattgaggaagccagagcatatgtgcgcacagaggccatctgtatctgtaaccttttcctggtactgtgggtgtgtgcggtgtactttttgtttcgggggtactctcatataggttcccgaatgagaactacttaacctgtggacccctgaaagtaaaattccttttttaaaaaacaacaacaaacctgttttgagagaggttcatttcagtacctattggtacaggagttaaccttctcagttactgggtccaatttcatgtgtgtcaggaggacactgtaacagatgtcagttactcaggaacttgagttgttaggaatcacaacttacttggggaagagcatttatcacattaatcattttataaaagttgtgattttttccctttcacccccccctttttttttttagagtaacaaattacagcacttttttatttggtttgaggctttgcttttttgaattatctttatttctgtatcaatttaggatacatctttgatatttaagtcaggaatgattttttccttccattattttaaagctgaagtttataaaacctttgacttgtattttaggtatcttgtcagagtaccaaattaaaggtttatgaaagtaattaatatttcatatacagaccttaagggttaaacatagagcagaattttatgtaagaagacatagacagtagtatactgagaattgtggtccgtctagacaactgtgggctcgttctgtctgaagccactgctgagagatactttgtgggaatgaatgtaagcctggggttatctctcttttcgaagttctccattcaggatcttgattagattaatttaaaactttctcaaacttaacaatgtttggtccttttttttaaagtaatgagttctgcaagtttattagcctgtgtattatgtgacagttcatttacatattccatacctgtgtccctaaactttcaggacacagtcagagtttgtgtttcaagaattatcaggacaagcctcagttcatcttttcttaattttatggactttcattatttctaccatcagtttctccaactgttgtcttagaataatgtctttcatttgaccacttttccctcctctggaacttctcccactgaattgtttttctttagttgccttgttatgctttggtaaggaactttaaattctgtcctttaaatgtgtactgacatgaaaatactgaatagtggatgtaatgttagaatcagagtcacgtattctgtttttcactaagatattgcaaagagaaatgtagtcattttctaaaatactgtacccaaaatacagtagaagctgtattttgggaaggaggggaggacaaactaagaaacaaactatagtttgaatgtaggttgcactctgtcaaagttgcttactaactttaggacctacttttattgcttatggattggttaaaaattacggtattttttttttttttgagacagagtcttgctctgtcacccaggctggagtgcaatggagcgatcttggctcactgcaacctccgcctcccaggttcaagcgattctcatgcctcagcctcccaaatagctgggactacaggcatgtgctaccacgcctggctaattttttttgtatttttagtagagatggggtttcaccatgtgggtcaggctggtctagaactcctgacctcaaatgatctgcccacctcggcctcccaaagtgctgggattacaggcgttgagacaccgcgtcctgccaaaaaattatggtaatgttttattgcacgtttgggtagaaatgacaaagtttttggggtccctgcctttgagatgcttttacttgtaggtggtggagacaatcaaacacaaattgcacaaagctataggagaggggggatagaccaatatttatctaagaccgcactggatgttagatattgtcaacttatagatgtgaatatatttttacatttttcactagggaaatgttgcttttggacgtgtacatatagaactaattgaagtattgaagccaaatctgggaaggtttcttttaggaagaaaagagcaagtggagacacaatatttaagacttttggaattgaaaggcataagggacagataaaattacgaagctggtttgaagggaatacagatagaggttgttgagtagtgttagggtcagttgtgtttgtgtagacacatgattacagttcaaagaagcaactgagaagagtgttcctgagagtaatttcagatgactggggagaaattacattttaggtaaaagctgtatgcctggattaggattttgtttttctagggaaccaacatttttattatatcgtggctatccagggctatacagaaggaggagtgaaaaagaagatgcttagtgtgttgaaagtggcttcttacaaaaaagagtttagaaatgggacagtagactttgctttggatgtgaaattttagggcaacaattggatactggtgcagtttcaaaacagcaaaatagtttagcaggtaatatactgaaaatgtaaagttggaattattttcaaagtatttaggtttcccagagtactgatggaaagtgaagagctgaaaagtgggtatttttagttagggggaaaatctgaatgaatgagacactacctaaaatagatgttttagaagtggataagagttgaagttaaaaaggaatgagcaggctttaaacatggccaccagtttagtcccagggtatattattctgtatttttgaaacaagctaaaatttttttttttttttaaatagggtatataggtcccttgagattaagttcagaagagtatacttaataataatttcattgttaaggaatttataaatagaaggacagatttgtttcctcaaattcatgttttaaagttcactgtgcattattatcctacgtttggtcttgatatgacgaagtgaggccaaaaatagctgacagagaaggctgcttcccttgttcacagttggtgtgtgtctttgtgtcccatacaatggaggggatatccctgtgtaggcacagtagtgctgaacatgctttttttttttttttccttttaaactttttattaaaaaaaagcaacttggcatttaaaaaaatgtagacatttttgcggcttgcatttagtattgactttaacaggattaaggattgtgaggaaggaaatatatattcacacttttttgtgttccttagttggaattctgcaccttcctagtagcttctctgacttcttaacagtgattattcaaatggcagaaactctggggaagagatgtttcaacaaggagataggtctaaggtgattctagggtgaaaatatagggattggattatatgccatatggaggccttctcttgtcatcctccactttccacaacattttttgtgcagtcgaagccatgttcagatgaattttagaaggcagccacttatagttaatgttatttactagtgaggacagtgggaagagtggtaacttcatgtactttgccttggcaacttttggattgatagggaattagagagccttgagggtatatgtttgtgtgtgtgtgtgtgttttagcacccaccagatgaccccaaaggccatgcataggctcatttcattgtactttggagatggttcttggagaagtcttctggtgcttctgacatctcattgtgtagagaaagagttggcaaactatttttgtaaagggccagttagtaaatattttaggttttacaggccacatgatctgtcacaactttcaattctgttcttatagtgtgaaagcagctgtagacaacaaggaagtgaatgaatgttgccgtgctccaattggcccataggccgaagtttttggactcctggtatagacaactgccaaatccaccaacaaacagtatgagctttattacactacaaagccaggactggctatttttggtggtctttgttttctagatgagatgtttttctctgagttcatttaaaatgattctgcataaagcacatactctactgtacaaaatacttttgccttcatggtgatgaaaactatggattgagggtcggggtgataccatctttaaagggatgcagctaaaaagtagggactacctgcatttcttatctaaagataaccacagttggcatttagtgatatactggctttgtatcatggccaaaagtatctgtaaagaagcttgtgggcatacttttgaaaccttctgaaaataaatgtaaagcatttacttcgtgtgtaaaattagaagtgtctatggttggtttgaataatatgagtcaaattataagagcattaaagtttgatactgctatctattaaagtcttaatctcttgcagtggctaattcagtgtgtagaattacacatcgcaatttttgttttgttaatttcaagtactagtgtatattaacaaatcacttaaatcatttaaatgtgtactttaaaaagctttgatgtatatacatatatatacgtatatatatatatgtacacgtatatatatacacgtgtgtgtgtgtgtgtgtgtgtgtgtgtgtgtattttttttttttttgtgacggggtctcattctgtcacccaggctggagtgctgtggtgtgatctcagctcactgcaacctccacctcctgggctcaagtcatcctcctacctcagcctctcgagtggctgggatcacaggcatgcgccaccatgcccgactaatttttttgtatttttgatagagatgggtaacaacttttggattgatagggaattagagagcctcgagggtatatgtgtgtgtgttttagcacccaccaggtaaccccaaaggccatgcatcatgttgcccaggctggtctcttaactcctgagctcaggtaatccatctacctcgacctccctcagtgctgggattgcaggcgtgagccaccacacctggccagcattgctatatttttaagagtgaatctttctcacatgtgttgttcagaaagtgtaattcactttgtgcaattttacattacagttaatattaaaatatttgtttcatatgtcagcagataccaacattggatgatggtatatttagtttcataaatcctatttattttaggattcccttgtttatggcaggggtggagattactcttgcaataatgatcactttttagttagtttgaaacctgcttttagtttatatgtagtatttctacatattgttacttaaattgaactaacaatttactttttaatttttcttcttccttttaaaaaaagagtatataatctgacaatgttagtacatcttttagaaggtcactataaagttgctttccctatttatttggaaaggattatttccttattattttaaaaaatcttttaatttttgtcttttcaagtaattttatcattgtcccaagcctaaggatgagtggcaatttaaaagacacaaggtgtgcatcttctatctgcaaatactccaaacagaaattattccagtttgttgatactttgagtggaccagggaaaaatgtgtatgtttttagtgtaactgaattgtcattacaaaaagatgagtaaagttgctgttaaataatgaattcttgctgttaatttgtcctgttcttagactttcccccattaagtataatgggggaaaatgtacacacacacacacacacacaaagtatgtatgtgtagatatacctagcgtattgtgctagatgttcatttctgcatgactgctctggaatttaggcaatattaaaagaccttacaaaatatttttaatatcacttaatatcctgttttatgtcttaatgtacttttatgtttttgctgctttctgatgatacctccttggtacttcctcatgatacctttcacagtatgtttaaatggaatgtattttctcaaagtacgtgacattttctgaacactatgggatatatatcttctagtaattatatttttacatgcaagtatctttaaaaatttcttcctattacaacttaagaattgtgaaacatttgagggagcatctgcagtttgagcacttgatctggatattggaatgacaaacttaacatttaactaaatttttctgaggtgttcttttacaatttttgagttaacacttgatttctgtaaagcagaaggcttataagaagaaaatatgagagccagtgttcgtcttgtattatcctgccatgcacgtagttgaaaatcagaaacaaacagaaatgcacaagagaccttattgcttgagtatcttctgctcttcccccatcagacctaagggacagctagtggatcagtcagatactgtgcagtttctgttgctatgctgtgtcacctttgccctttctttctatctggaagatagctactggaaatctggctgtaagaaagagatagcatgtttttttcctctctctctctgtgtgtttctgtctgtctgtgtacctgtacctagtgtattgtgttagatgttcatttctgcatgactgctctggaatttaggaaatattaaaagactacaaaatatttttaatattatttagccttttctctgagaggatcacatcctctggctttggcgttttcatctcacagcatcttaaacgaacccgtcaatgtttattatcagttccaaatgtgaagtctcaaggcaaattgttggtttgtttttggaagaatgtatgtgtgtgtgtatatatatgtgtgtgtgtgtgtgtgtgtgtgtgtgtgtgtgtgtatataatttttaaatttttaacaaaaattcctgatctagtcaatgcttatgagagtgtgtgtggtttgtataaaattattttttttagagacagggttgcttgttgtcaccaggctggagggcagtggtgtgatccttgctcactgcagccgtgaccccctgggctcaagtgatcttcccaccttagcctcctgagtagctgagtagctgggactataggtgtgtactgccacaccctgcaaatttaaaaaaaaaattctgtaaagacagggtctcactatattgcccaggctggttctcaaactccaggactcaagcagtcctcctgaaatggcctcccaaagtgttgagattacaggctggggccacagtgcccggtcagagtgtgtgtattttttaacagccttttaggattgcatgctttttaggggggtgaatgaggggtaatatacctttgcatgacattctctaggtctgagaaggagttgcttcactaaaagtgtaatttataattaccaaatggttgaaaacaggtctacctggagtcagcaacaggcagctagtttacttctcatttacgtaataaaatacattcataatatgcattcagtaattcataattcaataatttcttggcttcatggccttggacaagctatttattatccttttaattttggtggcatcacctgttgatggaggaaaacgtgtcctttacatggtggttgttaggattaaatgagataatataaagcatgcaaagctcttaatccagtgcctggcatatagcatttgacccatcagtgttaatttattaagttaaagttgttttaaagtgtgttgtagatagaagatttaaatcaggccttatattaagcccaacatttaaattcaacacttgagtttgacaaataatactgaagtaattctgtgttttcagaaacatctaaaacttcattaacattttattaaaaactgaagaaatgacagaactgttgggcttttttttaatagaaaagaaagactgatttgaacataactggaatttgaatttagtttcaaagatctcagtagtgcatggaccagaaaagaatccgtttttgaatagttagtgattatgatagtgactgtggtttaaattgcccatttagaaatagcaggatctaattcagttatcagcctttttagttgcccatctctttaacccctggaaataaaaagctttaaaaaatcattaagaagttgaacggaaattttatctgtgtcatttacatctcaaattaagatcgaaataattttgtttacatgattctattttaactacttaattttagtttttgttttttcctgaaattattagtatgtgggttttaaaaattcctttttttgtgtgcttaatatttgttaaatacaagattggttttcagaaataccttttcttcgagtagggcataatcagtgttttattggccaatacaataaataaatgcttcttgattgtttttgtatttggaaagaaattctatgttgagaacagttttggtatgctttccttcattggctgtttattgaatagaactgtatatttaaggtatatgaaaaaaatctaagctagaaaaaaagactgagatacattcttggaagaaaatacttgcaaactatgcatctgacaaaggtctattgtccaacgtctataagaacttaaatttacaagagaaaaacgaccccattaaaaagtggacaaaggacgtgaatagacacttctcagaagaagacttacatgtggccaacaagcaaagaaagctcaatattactcattagagaaacgcaaatcaaaaccacaatgagataccatctcagactattcagagtggctactataaaaagtcaaaaaataacagatgctagtgaagttgtggagaaaagggaacacttatatactgttggtgggagtgtaaattagttcaaccattgtggaaatcagtatcgcgattccttaaagagctaacagcagaactaccattccacccagcaatcccattactaatgggtatatacccagaagaatataattcattctaccataaagacacatgcacacgcgtgttccttacagcactaatcacaatagcaaagatgtggaatcaatctaaatgcccatcagtgacagattggataaagaaaatgtggtacatacacaccatgggatactgtgcagccataaaaaagaaggagatcatgtcttttgtgggagtatggatgaagctggaggctattatccgtagcacactaatgcaggaacagaaaaccaagtaccgcgtgttctcacttataagtgggagctaaatggtaagaacttatgaacaaaatgaatagacactggggtctacttgagggggagggttggaggagggagaagagcagaaaagataactattgggtactgggtttaatacctggttgttgaaataataagtacagcacaccctcgtgacatgtgtttacctatgaaacctttacatgtaccccgaaacctaaaataaaagtttaaaaaatatattctttagcactgattatcttgctagctgtgacttctccttttaaggaaagtttgctttagattttggtgtaaatgtagaaattagatgggtttatctgctagtggctctctagttagatgattaatgaatattctagtttaaaaaatttcagcattttaaatacttttctttattggtaacgatttggattatgcttctagggatctttacatttcatagtttttcaaaaaagactttataatgagttcctggatgggaagaagagcatgttctctctagtttatttgatttcttcctgattattatgtatgtaatactggaaggttgtgtaaataaatatttgggttgtaagttttcttttgcatgattggtggtctcttaattgactgctgtgtgaatttgatagtataaactagaatgcttatacagtgagtggggaagcctttttctactgagtgttatgcaggtggatttttcccccctgcggataaatactaaacttttatattaccagatttagacatttagacaggaaacaaaatttccttttttttaaaataaatgagcgggagaagagagtggggttgtacagaggtggggagagaggcagtaaaggagggaggaaagaagaggcagatatcagggagagtggctccggaagttttgagttcagtcttagtcagaggtcacaccctgttatttctttgggttgtggacatgaggtgttaccacttgttatctgtcaccctttgaaatgatgattagattattggtttttattcatagcttttcaacatttgggttctttaaaccattgaaggtttagtttgtatgggataatcagtgttattttagggtgggaaaatttccaccatacctgctaggagaaggggatggccaaagagtttttgacttacagaggagtttttttgttctgttgttttttcctcattgactctatttcgctagctttgtctgagaggggtgcttataattctatatgtgacatagtagctgagaggcaagctttggagttgatagacccaggttccagctctgctgctttgtgatttcaggcaagttatttattccatgtgtgtttcttttaaaactgggatgataataatagtacctgtttcccttatagagtaattaagaggattgagtgagataatgcatgtaagcctttagcacagtgtctgcctaagttagcatccagtaaatgttagctgctgcaataataagaaatattatagtattaagttattgttatttctgtgagggtgctggttttctgaatgctgagacattggtgatctttgctcctgaacgtgttactatttggaatactgcctttattcagcagcagattcagttcagatttactgaacacctactatgccaggaattgatctaaataagcaaaacaaaaatcctgctctcttgaaacttactttctggttaggagagacaaaatacacaaaacaaaatagatgatgtgtcactcaggaaatttggttgttggactttgtctaagctttcattaaccaaccctagcagtcttaacctttacatactttttgattgttcctgttgctgtgcttgagagtgtttggaggtaggggcaccctaaaagcgtatcatagactagctttattgtctccagcttcagcttgatcctaattttaaggcagccttaaatgatagttttcatcatttccttgacttactacttaaattggctatctcaaaactttcagtccccaaaacttgtatttcacatcattcagagagatgttgctcctccccagctgctcctcttttgctcttctatgccattttcttaagaggaggggatactagaagcatctttttagatttattttctgactagtctaactgtatacttttctttctcatattttgtgttttctaccctttatttattgttaactgatacatttaatttgcttattttctttaaaatatgtgttttttttggtctctccaattaaattgtgaaagtttttaggtcagggaccacttttctattttgaccttgagcttactctcttgaaaaaaagataataccacccagggatgttgtgagacctcaaagagataatgtgtgaaaggggtcctggagccagatccttaataaatgttagtttcttctttgaaccactctagttgctgaaactgctgtgcatataatcagtgcttaaaaaatgcttgttaatttatcttgaattgagtttgtgaactttcaattgtcacttggtggtagttttagtttttagtgctagataagattcaaaatactacttttcaagtgtcagatgataaatgctgggtcattagtggtagagctcaacagtgattaggagcaggaacttgagccatcattgtttgggttcaagtacaggctaccacacttactggttgtatgacctttggcaagttgtttaacctccctcggtttctttatctgtaaggaggggaaaatgatatctacccacctcatggttacttctttgttagtattaaacgaagtaaatgagaagctcgaaacagtgcttagtatataacaatctataacagggtccccaggggacatttggcactatttggtgatgtttctgatggtcatttgggggagtggggagagggaagggtgctactggtatctcgtgggtagaggttaggtataaacaaggtataggacagcatccacaacaaaaaaattacctggtccagaatgccagccctgccaagcaacattgggaaacccagcttagttcctttgtataaggtactttgccagttagattctgtatacatgctgattattgataaagatgagtcacagacatggataggcttcatttaacattcattttaaaagacagacatgtaagaaggttagtaggtttcatttctttttaaggttacagggaaagtactccaaaattaatctaaaagtccttacacttttttcagaaactgagaaaaggttccagatgaattttactaatttaatttttttatgacgagctattcatgtaaaagtgtattatgaatagagggcttatgagatatgagtgccgtagtgtgtattcataaagtcaaacgtacacttaaaaaacaagtatcaaggtaaatggaatgatctattgggggaaaccttggagctctctctctttccatatgttttattactcaccaagtcaagggctctttaacttaggatcttgtttttctgtgtacacttttcctgtttactgtttaaaattggacccttactaaaaatacagaaaattagctgggcatggtggcgtgcacctgtaatttcagctactggggaggctgaggtagggagaatcgcttgaaccagggagacggaggttgcagtgagctgagatcgcgccattgcactccagcctgggtgacagggcgagactcagtctcaaaaaaaaaaaaaaaaaaaaaaaaaaattggacccttaccattccttgtccatttcaggagcctcttaattgccttgcttctgttctttctctgtattcccacttgaattgactcttctttgctgggatccctttgggatcccttctagtccagatccaatctacccttcagagctccttccaccgtccccttatttctgggagagaattcagccttaataccctaaggatggaatttgaatgtactgacttttctgctatttacagggttgtgtattatctttggaagaattgggaagtcatatcattttctgtatgttgtggttcaggtgacttgtttcacttgtatccaaacactggttgtgtgtccatgtatatacagcatgtatgtactttaatgtctatttgtatgtgtgtgtacatgtatattctttcatgtatatttgtctctacaccttgctgtttcctctgcttggaatattcttttcttttcatctgggaaacatttgtctcttcagaccctacttaaatattgatctcctaggaagtgtgtcctgattaatataactagaattagttatttttttccactggttacccacagtatctctgtggcttttatagcacttaacatccttatttgtatttttcttttctactgatctctcagttctttgagggcaagagtcttgtcttagtcaacacctgctcccccaagtacctaacattcctgccacattgtagacatatagtatgttaacaaatgaatgggagagacaaaaaactggaaagcttttaaataagttctatgggggttggcaggaggggagaccacacattatcttgtcacctttgtttccaagtgcttagtgttgtgcttgcccataggtgttcagcaaatatttgttgaatgaattagaagcaaatcccaggagctgataaaattttcatgtgaacttttttcttctgcatttctagaaagcatatcctagaagtaaagtagcttagatattttggaacatcttattcttaaagtcgaccttcctatttttgactcagtagtgaaactttgatccaatggatacttcctgaatagtgcttttggctgattcctttttcacaacataggtccgttctggtgttgagtttgttggaaagatgaatatgtagtatcatttttacatcatggaggagccatcattaagtaacagtgttaaataaggactggtcccctttgcacatttgggcccaaattaagtgagtatgtactcttaagtatgggtaatgattgaggctattgaggtgtaggaaagaaagaggacaactggaggtgaagaagtgacctactagatctcttttgcttttggtaatgattgattttttttttttttttttttgagacggagtctcgctctgtcgcccaggccggactgcggactgcagtggcgcaatctcggctcactgcaagctccgcttcccgggttcacgccattctcctgcctcagcctccccagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccggtaatgattgattttttacccccagttatttttaaagaattggcaaggaaggagaggggtatgaatctagaagataagaatgtagattttgtaattcatgatcattattattaccatgtattgaatgtttactgtatgtcaggaactgtcctaaggatttttacaaccgttatttaacttagtcctcacagcagttcatatgaaatagctactgatgttttatggcaaaggaaacaaattcagagaaatttccaagaacacatcggaattttgacttgcagaactgggattcaaatctagatctgtcatactccatggtctatgcttttaatcatgcatgctgtctcttaagtttttattgaaaatggaaagttgtactgacataggaggggaaaatgatagaaaatcatcctattggaaggcagattaacagactcgtgaagaagcaattcccagttttatgtcattatacaaaataattgtgaaattgaagtccgtcatttgaattgatttgcattaaccctcattttttagtcttgctcttggaatcatcatcaggacaggtgtcccagatatgttagcagtagaataaatttatctaggcaaaatctgtctaagattatggagaaaactgattttatatgtgaaatgtgttgttgagtgagagttaaatcttttgaaggagattatttcaggatgagcctcaaatacattatttaggatttgaaaacctatggtttagcagaaaatctgagaatcaacatttgttagtctcttgggtataaattgcttaagtcagtgggtatctgattattctttctttctttctttatttatttatttatttatttttgagacagagtctcactctgtagcccaggctggagtgcagtggtgtgatcttggctcactgcaagctctgcctcccggattcacgccattctcctccctcagcctcctgagtagctgggactacaggcgcccaccgctacgccctgctaattttttgtatttttagtagagacggggtttcaccgtgttagccaggatggtctcgatctcctgacctcgtgatccacccaccttcacctctcaaaatgctgggattacaggcatgagccaccacgcctggcttcccttatgtaatttcttaaactatttttgtttgctgatttggtgtttgtaatttaaaaaaaatttattttttcttcttttacatactttgtagaactattccctacccagtgagtttatttatagatatttcatatttaattaatactgtgagaacatagtttttatatctgtatttttgttttttcattaggatatttaaaataatttttgtaattctgttaccttaaaaagagctaaacttagatgttggcctttaaaaatgttttgtgaaagattttctcttggttccatttaaaatactgtatttgtaactatagttaaatacatttgaactttgcccttttcagaaattttaattatatgcctttataaaattttatcaaaagttacatgcaaatactaagtatagagtgctgaagctgagcgtgatggcttatgcctataatcccagttactcgagaggctgaggcaaaaggattgctttaggccgggagttggagaccagcctgggcatcatggcaaaaccttgtctctacaaaaataaaataaattagctgggtgtggtggtgtgcacctgtagtcctagttacttgggaggctgaggtgggaggatggttttgagcgtagtagttcaagactgtagtgagccaagatcctgccactatactttagcctgggtgagagagcaagaccctgtttcaaatatataaataaataaaatagtaggctgggtgcagtggctcatggctgtaatcccagcacattgggagtccaaggcgggtggatcacttgaggccaggagttccagaccagcctggctggcatggtgaaaccctgtctctactaaaaatagaaaaattagctgggtgtggtggtgcgtgcctgtaatcccagctactctggaggctgaggcaggagaattgcttgaacccgggtggtggaggttgcattgagctgagattgcaccattgcactccagcctgggcgacacagcgagactttgtcttgaagaaaaaaagaaaaaaataagtattgagtgctgaatgatgttctataaaagattaaaaatgcctaagtggaactaaattctggtaaatagaatttaggggttggggtagaaagaatcagataagtacttcaagattttcagactttaagtctaaggctcattagtatcctctgacataatctactacaggtttcctttcttagaagtgaatgttaatatggcaccactatacaaataatatcgatatcggtatttgagtgtttattatgtgacaggcactgttctgtgtccttcatgtgtattacctcatccagtcctcacgatagctctatgaggtaagtgttgttaatatccctattttatagctgaaaatttggcatagagaagttaaacatggtcaaggtcccacctttagttaatagtggagctgggattgtttccaggcagtctggctctagagcctgggaccttaatctttattctatactgtctgtgtcatctgtaaatttatttcttatatattatttatagaacagtgcctggcacacaataagagccatagaagtactggctattttcattggcatttaattatatatgaggttggcagagcatgcattattattccatttgtttatttatactattaattagatagtagactttattcagatttctgtgtttcctttttttaaatagtcgagtttaccgaagttcttagaaactggatgattgcctccaataatgtggccatgctagatctcaaaattaacttttttaaaaaaaccacccctccaactttatttacacttccttagaaatggataaagaccaagattgggcttctgttttgatatgtcattggtttagaaaggcagaaacagcctggcacggtagctcatgcgtataatcctagcactttgggaggctgaggcgagtggatcgcttgagctcaggagttcgagaccagctggacaacatggcgaaaccatctctactgaacatacaaaaattagctgggtgtagtggcacttgcctgtagtcccagctacttgggaggctgaggcaggaggattgcttgaacccaggaggtcgaggctgcagtgagccaagatggtgctactgcagtccagcctgggtgacaaaataagaccctgtctcaaaaacaaacaaaaaaagcggaaacaaccatactaagtttacagtattgtttgtgtgcctacttggtggaagaaataaattcttagaagttacactttctttaaaaaattaaaaattaaaaactgtgcagtgttgagattagtattaccgcttatcttgctgacttgtgaaattgaattacatcacttataataatgtgaagttttagaaattgttttgatgataaagagtagcagcctttaagagaaataatataaaccatgtttcctcaaatctaaatggtcatctgtatggtattagttttccatcgctctacagaagcagtccccagcctttttggcaccagggaccatttttgtggaagacagtttttccgtggatgcggggaaggagaggcagtggggaatggtttgggatgaatcatcaggcattaggttctcttaaggagcacctaacctagatgccttgcatgcacagttcacaatagtgtttgtgctcctatgagactctaatgccacagctgatctgacaggaggcggagctcaggcgataatactccacctgcagctcacctcctgctgtgcggccgggtttctaacaggccacggaccagtaccagttgtggacccctgctctacagcaaattaacacaaatttagcagcttatcttacagtttctattggtcaggaatttgggtatggtttagctgggtctttcctcaggttctcactaggctgaaattaaggaatcagccggggctcctatctcttctgaggctctgggttctcctccaggctcaccggttgtgggaagagttcaattccttgttcttgtagatctgatgtccctgtttgcttgctggctgtcagccagggaccactttgagctccttagaggcccacccccgccaccatgttcttgccatgtggccaagttattatgttgtgtatgactaaggaagaaaaatgctggattaaaatataagacaaattcttactttatgtatgactaaggaagaaaaaatgttgaattaaaacatgatacaacacattattgcttggacagtgccttgcaaaaaaaaaaaaaaaagagagttgtggttattcttccagccacaatatctgcttcagaaatgtcaaatttattttctactgttgtgtttgtgttcttttttgtaaacaacttttttgtttcaatgctgaatcatagtattttacaggacatttaaaattaagaagtaagcgtgacttcttattgtactactaaatgtacagaatttagtaataattatggtgatataaactgccattacccaatttgcaagtgtacagggaatgacagctatgttgtgactgccatgggcaaatggtgatttgtaaagcattgttgcttttttccccccattttaagttgattttattgagatataatttatatacagtaaactacaagacatttctgtcaccctcaaaagattcctcatgccccttcacagttaaatccttccctgcaccctcgcttcaggcaatcaatgatctgcttttttattactacaaattagtttgcattttttagtattttaaacagtgaggaaaaaagaggagatggagacaggctatttttaatttttatattttcttctttgttcttagagaaatcctagtttctttattaggcattaggtttattccttcagtgtgggggtaaagtgattcttcttgaagctttctaagtatgtttcctagatgttcttgacctctgttttgcctaagataacttttctccagcatacccagagttggctctcttaaaccatacgtgcttgtgtagctccctcatccatatttctacttattgtcttttcaggaccgtaaacagaggtatttttgacttgacattctcagctcctaaataatcttatagtgttactgaggcttttcacctgtcacatacccccgtcctagtctctttttgtgttgctgtaaatacccgaggctgggtaatttatgaagagaagaggtttatttggttcatggttcctgcaggttgtataagaagcatgatgtcagaacatctgctttctggtgagggcttcaggaagcttccactcatggcagagagtgaaggggagcaggtatcacatggtgaggcaggaaaggagagaaagaggagagggggtgccaagctctttttatcagttctcatgagaacttacagaggggaaactcattcattaccttgaggatagcacaagctgtttatgtggaatctgctcctatgacccaggcacctcccactgtgccccaccctcaacattggggatcagatttcaacctgagatttggaggtgtcaaatattcaaattatatcatcccctagagaacatacctccccacccctgagaatctcagaatttcttgttgagaattcatattacagtatgagttgtaggaaacatgaaggaaagatttcccttctagtcagttcttatatttatcacttgtttcatgctccttggtgtgtatagtgttttcaggtgcaggaaatgctctgggaagaatgttgcttcttagaacgtaagagaaagggaaccccaagggaagactcaaatgaagtccttaaaaagcccacttagcttcttctgagacagcattgtccaatggaatgcaagcgagatagttaaatttaaattttctggtagctatgttaataaagcaaaaaaagaaccagccaacattaattttaataatatattttatttaacacaatatatcccaaatgttattttaacatgtaatcaatatgaaaaccatgaagactttgatatttaaaattcttattttaatactaaatctttgaaatcagtgtatattttatacttacaagacatctgaatttggacttagtcacatttcaagtaagtgctcattagttagtgactaccttagtggacagtgtagatgtagattatgaactttttaggaaaagacaatagcatgtggactccacaagaaattgtctttaacaactgtttgactctcataggttagggctttagtgtttcagaatggtttctatggttgagatgtaaagttttttatttttataaaggtaagtacatttttatacattttcattatacaagtaatgtcattgttgaagagttggaaaagtctgggtaaaagacaacaaattgtcctcacttcctcagtccagagataaccaatggtaaactttattgaatttcctctttgggaatgttgctttttaaaatcaaagagaaaatagatttcttttttgtcataattccttcctgttcacattcagacatacattattattgacaattcttttacctctttgtttaaatttatgtattctttttctcgggctgtatcctccaatagggtctctgtgagtggagtactctggagcacagtaacctgaatggtgcttccctggattagggttaacaatagaaacttcaatggcttttatgtactcattgctaaaccagtgtagttgctttctattttaactatctgcgatctctttgcatatcctgcagcttttttagttattgaggtataatttgcataccagaaaattcactctattaaatactcaattcaggccggcataatttgcataccagaaaattcacgctattaaatactcagttcaggccaggcgcaatggctcatgcctgtaatcccagtttgggaggctgaggtgagcggatcacctgaggtcaagggttcgagaccagcctggccaatatggtgaaaccccgtctctactaaaaatacaaaaattagccgagcatggtggtgtgcgcctgtagtcccagctatttgggaggccgaggcaggagaatcgctggaacctgggaggtggaggttgcggtgagccgagatggtgccatttgcactccagcctgggcaacaggagcaaaactccgtctcaaataaataaataaataaatattcggtcgttttcattgtattcacacagttgtgcagtaattatcactattaatccagaacattttcattacctcaaaagaaccctgatgctcattaaggagtcacttcccatttcctctttgcgcagtctcctggcaaccactaatctacttcctatctctatggatttgcctattttagacatttcttataaatggagtcatacagtacatggccttttgtgtttggcttctttcacgtaacacgatgttttcagtgttcttctgtgttgtagcatgcatgagaacttcattctttttaggactgaatagtattcagttgtatggagggtaccacattttattcatcagttgatggactttggggttgtttctgctttttgagatcacattcattcttcaagaacattaatttcttaatatcgtcctccattcttatcaaagctttcagtggtttcctgttgtctataaacataatagaaaaatatttagtttgcattcagtcttacaaagtgtaacttcaagttactttcttttccactcttattttccatcttgttgttgtttactgtgtctaaaatatgtgctatatatgaattataaaattatgtagacaatatagataaccttgtcatctgatctaatttttaaaaagtgaatttgaatactgccaaactaaggagtatgtgtagtatggagaatttgagaaagacaagtatcagtaaagaagaaacagtatagtaaaacaaaatttttttctgccttggtccattgaaagaaatatttagtttcatataatgtgaacaaaaaccttaattttgaatgaacatttgatacataggaactcattcaaatcattgattttctggttaactttgttagtagtttgtttttttctgagtcttttgttttaatccagatagtcagtctttttctcctttttacatgcttgaaatggtaaaaatgaaatatttttacttattcttaaaaatatttccagaatgtcttacacacacatacacttagaaacaaatacaaatatatagtgatattctctcacattttacagagaaaagcgtagtatacatactttgttttttaatttcacagtagatcttagaggtcttttaatattagcatactgagaactttctcatttttttcataatagtattgtttattccattgtgatgaatatgctattgtttttgaaactagtcccatattggtggacatttaggttgtttccaggtccactattacagataatactacagtgaataagtttgtacatgtatcatttcatatgtatgccaatatttctgcaggataatttcccaaaagtgaaattgctgaatcaaaagatatatccacctgaaatttgattacccatatttttaagcataaaggaagagataaagggaaataaatagaaaaaaaagaaactatagattttcaaaaactttgtttatagatgaaaaaatggaggattagaacttttaagtaattgtccatggtcatgcactccacagaggactgggcatggtgggattcggtggggggggcggggggctgtgtcaggatgctaacccaggtgttcctgattttaaataagctgctatccttcctgttatattcatactgcatgttatgagcaaaaggggagagaagtcgccaactttttggagaaaaagaataaaatcccagactttatttttgggatcatctgaggtgggaagatggcttgggcccaggagtttgagaccagcctgggcaacatgaagagaccccctgtctctacaaaaaaaaaaaaaaaaaattaaccgtggtgtttgaggcttcagtgagctgtgatcactgcactctagcctgggtgacagaatgagatcctagtatatgtaggtatttaatgtatatcgaaggcagcatttcagattggtgggggagaagatggattatttggtaatttatattgcagcaactttctattcatttggggaaaaagccctgtcccttgctcttactctttacataaaaaataatttaaaaatgaaagcaaaaggtaatatcgaaggatgtcgtgaggttagccttattcaatagactgacctcagtgagtactgaactcatgaaagtttcatttttttcccagtcactgtcttttgcattttccttaattgatttttaacatcaccttggattaatctcatagctttcccaatatgtttttagttatgaaaagtctatttctcctcttgccccgtattaggcttgacctgtggctggagagatctgtggtactgtttcatatgtaccgccctcctctttctctcctcccttttttacttgttctgaaaacaatgagtaaggttaaaaaattatttttcacaaacatgaaaaaggttgataatacgtaagttttgaatatgggggaaagttactctcatactgttgttgggagtatacatttttgtggcctttttggagagcagtttgacagtatttatcaaaataaagtacacaggcatcttgaatcagcagttgttcttccaaaatttagtagtacagatatactgacataactttacttgaaagaagaatcctttttataacttagtgtttattgacaagagaattagcatttagagtcatttattattctaactcttctaccaaaatggtgagatgtgttgtaatttactggacgtttaggttgttcccaatttttttgtaagtatgaaaaatgctgcagtgaacatctttgtctgtgtgctacccagtcatcactgttgagaatgtttcttaatggctatatttaggtaagtgttatttccctttatttcccctcttatactttaaggagcacaggagatctcagttttaatcgcagctcaattgcctaaatggatgtgtgacttaaaacaagctctttaaccttactgagcaggaggttctgcagatatgggcctctcagactattttatagcagtaataaatggactacctattgtcacattcttcatgatcctgtagggagtatgtgtcagtaggacaacccattgagtgacagtcctaactttcttggttcctctccctcattgtgaggtcattgtttcacaattgtatatacttactgattcaaaaaacaactccaacacttattgaggggtagtactgttcccagttgagaattactgttgtaaaaattttggcaggaattatataagattattcatgattgttatcgacttatctttattccttcatttttttttaaagcaggaagcaataatagtttacgttcgttaacctcaatatctactctaaaacatgttgtttttcttgttattaccatgaaagtctgggttaacaggagacgagactagcttcttcacagatacatacattaatttgacattatagaaaaatgagactgtatcctgacttgccagtttggttttgattggtggtttcacctttttttttagactcaggaaaacggtggcaaaaatagtataaagggtaaattctcatatgcccttcattcaagttcccaaaatttaacatcttatgcagtgacagaaaaattatcaaaatcaggagattaacattcatgtaatactgatagtctacagactttattcagatttcacccattttctcaccaatggcctttctctggtccaggatcacaatatgaaattattcacgtgttgtattttagttatcatgttttcttagttttctttaacctgaaacaattccagtctttctttgccttttgtgactgttatactttggaagagtactggtcagtaattttgtggaatattgcacaatttgtgtttgtatgttttcctatgattcaattaattttaagcatttttgattaagaatactgcaaaaatgattttatgaatcttatttagaggcacatgatgctgatttgtccagttatcagtgatactaattttcagttcctttcgttaaggtggtatcaggtttgtccactctagttgctttttttcctttgtgattcataagtagtgttgattcagattctaggttaagaaaaggaaaaaaaaaagtatcctatgggaaggtacttaaaaataagtactttgggaccaggtaattaacatttctcaccatactctaccaatttttaacttccttgctgttttttttttcttttttttttttgcagtggcacgatcatgagtcactgcaagcctcaatctcctgggctcaggtgatcctcctacctcagcctgttaagtagctgggactgcaggtgcacaccaccatgcctggctaatttttcatattgtttgtagaggcgaggtttcatcatgttgtccaggctagtctcgaactcctggactcaagccatcctgtgttggcctcccaaggtgttgggattacaagcgtgagccactgcctggccccagactttaacttttattgatgattttgtcatggtgattttctgtttccatcatttcttctgtatttacattagtggtttttggtaaggaagaactttcccttgttatgtatttatttaatgttttatgtatatcagaatggactcgcggattttttttcccctcttatatcagatggacttacagattcttctatggatttcatctattatcatcattatttattttggtgctctaattatcccaggtttggccccatgaagcctcttcaaggaggttcctggtccttttgacatgtctccatcattttgtgagggcttaactttttttttttctttttgagtcagggtcctgctgtgtcactcaggctggagtgcgcagtggtatgatcatagctcactgcaacctggatttcctgggctcaagtgatccccctgccttagcctccggagtagctaggactacaggccaggtatgtgccaccacacccagctagttttcgccttttttttttttctccaatagagacagagtcttgctgtgttgcccaagctagtcttaaactcctggcctcaagcagtcctcccaccttggtctcccaaagtgctgggattataggcatggaccaccacacctggtcagaaggcttaactttctagtagcgcaaaatgttctagtctcttcttgtgcttttctctgccccagccatgaaatcagccatttcaccaaggaatattgattccttttactggaaatcggaattcttccattccccctgcccccagtacttgtatcttaccatctttttaaaagtctttgtgtcttagaaccgtgttagatgatctggacacattaccctgatgtacagaaattagtttatacacggtaattctttttgtagcagactgtttaatttgtagcagattcattttgcactgattaatcctggaggatttcttcacaagttacattgtgtcaaatatgttatttcatgggaacacaattgaattttttccttaattttgttcggtatttgataataacaatttaaaaaacttttctaagagcttccttctaatattatatgatataggaatgtttacatttctcatttatttatttggtgtagtgatctcagaagtttttttaaaagaaaaatcttgactttgtctcaggtactctcagttcacttccatgtgggagggtcatttgccatccatccagattacctttttgattaggcttttcagtgtctataaatatttcggtcttttctgatcatttaatttctaatacatgcatatgctcctatgtataataaatagacaacttcaaatttgcagtttctagatggttggaaaagggaaacattgtggtgtgtaatttatcagccatcagatcctagatatttgagattttaactaagcaaggtattagatagaccatgttgttttggcttcacagaattcattcatattgtgcattacacaatcagtgtgcatattgcacattgattttatctgtaagttgtctttatcagtggttctcaaagtgtggtcccctgctagtatagtatcagcctcacattggaactggttagaaatgcagacttctcaggatccacctaattgcagtagttaattttaacaagcccttcggtgatcctgaaacatgttacagtttgagaaacactgctataatacgtttcatttaaattgtttcaggttgtgggggtagggaataagactaccaatttattcatcttctgtgcaatattacctgtttacctaactcttagagatattaagatattttgaagaatgtgtcccatgagattataatggaactgacaaattcctattgcttagtgatatcatagctgtcatgaagtcttagtgctgtaccttactcatgtgtttgtggtggtgatggtgtacacaaatcttctgcactgccagtcgtctgaaagtatagcacatggccgggcgcggtggctcacgcctataatcccaacactttgggaggctgaggcgggcagatcacaaggtcaggagattgagaccatcctggctaacacggtgaaaccccgtctctactaaaaatacaaaaaattagctgggtgtggtggcgggcacctgtagtcccagctactcaggaggctgaggcaggagaatgacgggaacctgggaggcagagcttgcagtgagctgagatcgtgccaccgcactcccgcctgggtgacagagcaagactccgtctcaaaaaaaaagtatagcacatacaattatgtacagtacctaatacttgataataaaggactgtgttactggttgatatatttacaatactgcactttctgttgttactttagaatgcactgtttgaactttaaaaaaataaattcactttgggaggccgaggcggacgaattgcttgaggccaggaattcgagactagcctggccaacatcgcaaaaccctgtctctactaaaaatacaaaaattagctgggtgtggtggtgcacgtctgtaatcctggctacttggaggctgaggcacgagaattgcttgaacctgggaggcagaggtagcagtgagccagtatagcgccaccacactccagtcttggtgacagagtgagactctacctcaaaaaaaaaaaaaaaattgcaaaacagcctcaagaaggcccttcaggaggtgttccagaaggcagcactgttgttgtaggagattacagttccatgtgtgtgattgcctctgaagaccttccagtgggacattatgtggagatggaagacagtgatattgatgatcctaatcctgtgtaggcttgggttattgtgtatatttgtgtcttaaaacagttttgaaagttaaaaaaattttttttaaaacaggaaaaaggcttataaaataaggatataaggaaagaaaatatttttatatagctttactatgtgttttaagctaagtgttattacaaaacaggcaaaaaattaaaacgttcataaagtaaaaacgttatagtaagctgaggttattacggaagaaagaaaaaattttaaaataaacttagtacagcctaagtgcacactgtttataaagtgtatggtagtatacaataatgtcctaggccttcatatgcatccaccccaactgactcatctagagcaccttccagtcctgctagcttaatttatgatacagatgcaccgttttacattttatataccttatttttaatataccttttatatatttagatgtgttaatatcattgtgctacagttgcatacagtattcagtacagtaacatgctgtacaggtttgtaacctgggagcaatagtttataccatggagatttgtgtaaaagcactgtgtaatattcacacaaagaagaaattgcctgacacatttctctgaatgtatctttgtcatgaggcgatgcgtgactgtataatttgagccacacatgttattttaaaaattgcaatacccttattaatgtaaaaagcagttgaattaattttaagatatttagcctgatggagccatgatattaccatttcaacataatttataaaaaaaattgttattttagattctctttgtaccaagtttttgaaatctggtagatattttatatctcaatttcaacactaactttttaccggaagtaattgatcttcctttagatttcataaagtttatggttggaaaagtagattcacatatctaaatcgtttcagacacacttaagtatttcgctacctgaattaagtaccaaaaaataatttttctgtaatatttgcatctaaattggtaaaactggttcatgttttttagtagaatgtagtagactttgaagcaaaactgtatccatttcaaaattgagctactgaagttaagcaaattcatgaactcttgtgtcaactccgtattactgacattatattcaaaaggagattggatgttaggcaaatctatattaaataaagtgtttttcaatgatttttttgtaatttttgcagccactttgcaaaacactgtcaattaaaatttatatatgttgatttatgttagaacaaagtgtaaaatcttttttattggtttgtgttatgaaaactttaaatttcaatgtaaatttataagaaagttcttataaaatttctggcataccttcttccagtttctttttattttactttttcttgtgttagcctggtcataaataaactctacctttctttggtgcttcaagttaagcttgttcataggcattgagatgtaagtgaggaaacaaaagaatactgccatttttgtcttttattacttaatagtgaaaagcatctctttctaaaatcagagttgacattagagtagatctttgtgaagttattccactatacccagtgagcaaagaacacagatgttttaaatgtattgttttttatttttttaacatttccatgaactggctaggattcatatttttggcatgtacatactgaacaattcaactctttacatagggcttttcatagtctgtttcagaaagctgaacacagatattttcaatgtgtatcatacagtggaataaaggaataggagaaacatcaatttttgcttttaaaattcctaacatagctggagctgtctgttgtgatagaaactaattgtttaatacctagctgaaattctttgacagaggtaaaggattaaaaaatatctatgccactcttgattttttttttttttttacagctacatagtgacaactttttcttaagtttagaagttctttcaaataaatttcacctaaaagatttaattggggccgggcacggtggctcatgcctgtaattccagcactttgggaggccaaggcaagtggatctcttgaggccaggagatcgagaccagcctggccaacatgacaaaaccccatctctactaaaaaatgcaaaaaattagccgggcgtggtggtgtgcatctgtaatgccagctacttggaggctgaggcaggagaatcacttgaacctgggaggtggaggttgcagtgagccgagattgcgccactgcactccagccagggcaacagagcgagactgtctcaaaaaaaaaaatttaataaggtagtgtctcttatatcacatggctcatctaaagctaaagggaaatagtaatttttcacattttgaatctgttgatttttcgtattattagaaaggtcttccatatgatggatggtgtcttaattgaatatcttccacgtgttgtaaagtatctattttagtattcctcataattttctaacaaaatttctataattgaaataatttctttaccatctctttatgtaaatgtgattttctttcttttggcgcaagaatttaagccagtttatagctgaccagagttacaagcccagatttgttaaaaagcttttaaacacgtttgttgtacatttacttctgacattgtttggctaattttgttgcttttcttctgattgtagagcagaaagttcttataaaattcactgtgtatttgttgaaaatgtctcctaatattgtctactttacttgtaaaactttaaaacccaagaaatagcttttaattttgctctgtcacagctaattgtaattgtcattcattagaaaatttctagcataccttcttccagtttctttttactttactgttctggtggtagcattagcttctgtatctctacttgattgtgtattgctgtaatgccttctttttacttttaaaaatgtgtcctcctcttgtccattcatttttaaagtaagaaaattaattatattcaaaatattaaaattaaaaaaaataaaaagtattgcgattgagttaccagttgtgatttacataggcatcactgcaacttgtgttatttgtgaaaaacgtatttaagtaaacgcagtacggtgttagattgatgagtagaaaaatactcattctaactgtaaattagttaatttttactgactagatcagtatttttatgtgtaatactagaaatgactcactgtatcctgagatgtggagtataatatgcagtacagtggtcacagtgaaatgtagtactgccaaaagaataaaactttctttagtagaaggacattttacacagcttcagcttttaaatttaaattaagtacaactaaataaagctaaaaactcaattccctggtaacactagccacatttcaagtgcttagtagtcacatgtggctaatagctaatatattagaaattaaaccttttgagagttggttatgtatgtgattaaagtattttggttacttgggactagagattacagtcatttttgatcaggctgatgtcataggaacagtaccaaggggacttctgaatcaagcaccctagaaagagctacttagaactacttgcattttctttgtggcatctcttataggcatgaaaaaatttcaaacatttttcatggataaaaggatttaatcagaaaagcatttggatatacatatttgaaatcatagcttgcctgtatgttactagagtagttggagagtggaaaagtatctatattctaaagactgtttcatcatttgggtgaaaaaccaaaaaaagagagtaattttgtttaatagctctttcacaaataaaaaaaagaatgttcatctgtagagacctatcgagatctcatcaggtttacaacaaactttagattagcaagctcaatttcagttgacatggattgtggaaagattttcagtggagctcagatatttaatatcctgaatttggatgaatatacaatttctaaaattaagtacttcaactcctctgtggttttaaaaccagtagtggccatatcttgactagttattaaagcaccaacctttttttctttattactttgcttttatcctttgagcaagttaggcaataagttctccatgtttttgatttctacagaagtaaatgaatgttattgctgatgtttatttcattctgtcttgatctcatgatatgagaatataaggagatgtctgaaaacttgttttaaaaatcctatgaagcataagttcatgaataaagaaggaagtggaacttgtcttgagagaatttgcctatttagatggtttccaaagtgcattctgctagtcactagagatggtccttgaaagagttctgtaattaagtttgaagaaacactgtgtaccataataatggcctattgaatattttaccatgcacattaatatatcaaaggctttgagatatcttacaagaaagaaacttgctaaagcttgtcttcctgcatgtcttaagcttcttccacagtgactttaagtcggacttgtctatcctgtgagaaatgctgccctctagaggcacaaattcaattgctcttaaatttgagtaccataatgctgcctgatcttagtgtgaagttgtgataattaatattaatcaaatgcatggtaacaaaaccaaatagaagaaatcttatgttgcttaattttaagaaaatatgaggaaacagaattgcaatttataaatacccactttttaaattgtacatgtgatatttttggctgctaataatcttttgaatacatttctgtatatggggaattgcggacacatagtacttcaagtctccaaactctgagtctctcctgctagcagaagccatccttccctgtgttaacaaccttgcttaagacagttaccatccatggggaagctagtttcctcataccccttcccacgatactcatgtttccatacctgtaactggagtcagactatatcatgctgcacctttgcagtcacaatgggaagaaaaggattatatgtaatttttttaaagttttttttttttttttttttttttgctgacaaagcagcaaaaccttcaggaatatgtacagaaatgggtgatgatgatgatgtgagagagaaagaaacatgcttttgcatttggctgaatgtattggtatggtgcagttatcagagattctgaattcattgttctcgttcaagtagccaggagtggttctaattgtttgctcctttggtctcagcattgacttatactaaatgaagttgaatgcctaaaaactcatagcataatatagaggaagaagtcaaaagattaaaggaaatagaactgatggagtggatttatcactgtgaccattcacttacctcccctattccctctccagtattctctactaagaggtctggaagacacttcattcaccaacctaatgagaaatactttgagggcagtgccagcatatatgaaaagtaattcaattgcttttgtctgtaggctggaggagatactttcattgaaattgtcttttttacttcagggggtgtggccagattctaggtaaggtccaggcatcattttcataaggtagcttgacaggcatgatgatcagagatgcactgacttgaattctttttggctgtgactaaatgatcatggatccctacgtctaaaatagagaggaagcctattaagatcctactttatctgtatgagcaaaaaattttaggtctcataggagaaggccttacttgaactaccataagagtgagttggccagcctgggcaacatggtgaaaccctgtctctacaaaaaacacaaaagttaacttttgtgttttgggcatggctgcgtgagcctgtggtcctggctgctcaggaggctgccccctttttgcagtttacttcatgggttatatgagaaaacaggaaggagggggatatcaccgagagattctgatattggagctgtgtgcaataactgataagtctctggttcttacctccatatgaggaacagagagagtcttgtttgtacaaggcttagttataatctgtcaggtttgaggatcttgattggaagcatagacttgatctgtatttgtggtttctcatttcaaatagcctctcaatgttggataaatttgcatgttcatgttttatagagtcagaactcaatttcctagtattccttgcagctagggcacaagcatatgacctaggctttattgatcagatgcatctgtgtaagacatgaattcagggtcagagaaatgcagggtaacagctcttgcctggaattgctttctaatgtgcatgttgagtgacaagatattgagctgtttagaggtagcagtgacagagatactagtgagccttgttgtcctaggctcagctttcatggtactagggctgaaggatgggaacaatttggtggggagacttaggtatttttcctggcatcatatctccaagtggattcttcagttctctgataattcttggaatcccctaatatattaataaatttattttctcttgaaaccacctcaaatagattctgtttatagcttagagccctgattgatcgtctaactttaaagattttatttatcgtcaaaatattttatttttctaattgagatctaaaaattgtcaagcacttatactctattttttcccttcctagagctctgcaatcctttgctgtcattttttcttaccaggaatgccaatttttgtaccttttgaattgctttttcttctttgctacttggcttgagctccactattgttatgaaacctttcttataccatcccagcagcctgatggatttttttcttcctctgaattccagtagcacttgtttagacattaagtgcaaggtgtttttttgttttgttttgtttttctgtatgcatcttagcttccgcactaaatgataagctccctgaaaactagtactatgcctctttatatcgcttgcattgtccaacagaatgtcttgcacctattagattcaaggttcagcacagtcaccctcataatatatagtgatgagagttgctgagaatcacagtgatgccaggctgaatgagctgagataataaactagttagatgcatagtctggccatgaggcccaatttccatcttcctcaaggagtctgagacttgcctctcagtagttttcgttagtaggtgatcatatcttctgaattactgtcttcttaatgttttatgagtttttttttgccttgacaaaagccttctatttctctcatgcttcctagttccttccattgtgccctctgcaatttcttttcactaagcttactatttgcagtctcttaaaatttgtgtaaattatgaaatcaaagatacaaagaaaggacagaaagtagtataacaaacacctgtgtatctaacctcagggtcaagcagatattaaccttttgcctcgtttgatttccttttaaagttcttccctccccatacttttctcccttctttggaggtactcattatcctaaagtccttacgattatttccctgctgtttttgtacacataaaacatttacacaaaagatgacctgtgtttttttctagatgtgttcatattgacatgtgtagatttagttcattcattgtaactactgtttagttttccaggttgaataatgctaagttttatagagataatgcagttctaccttttggcaaataatattgtaatgctcgtacataggtacttactgtaattttatctaggatagatacctgaaagtagaattgctgaatcacagggttcaggtgccactttatcaagtattgccgagtccagtccaaagtggagctaccattttatacccattaacatgtttccacacttttttttatcaacatttaattttatctagctatgatttttttgcctatttgaaagatgagaaatggagtttgatgattttcattttcattactttgatttatagtaaagttgaatgtctttgcatagtttttgatcattctagtttcctattctgtgaatcttttatgttctttgctgatttttttcctattgggtattgaaccttaaattgatttgtggtgattctttttataatctggatattaatggttttggtaatatgcattgcaagtatctcatagtgtgtgtgtgtgtgtgtgtgtgtgtgtgtgtggtttttttttttttttttttttgagatggagtctcactctgtctcccaggctggagtgcagtggtgcgatcttggctcacacaaattctgcctcccgggttcaagtgattctcctgtctcagcctcctgagtagctgggattataggtgcccgccaccacacctggctaatttttgtatttttagtagagatgggatttcactgtgttgtccaggctggtctccaactcctgacctcgggtgatctgcccgccttggcctcccagagtgttgggatgacaggcgtgagccattgtgcccggccttcgtgtcggtttttaacattgtttaaaagtttcagatttactacatgagttcatttgtagaatctctcttctgtatcatagcttctttggtctgctttcatgtctttacaatattgtgttaattttaatagttttgatataaatcttgttatctgataaggtgaatcttctctcaatgaaaattttgggtcagctaatcatatttcaccctgaccctaatgaaaatttaacagaaaacaaaccctgttggaaatgtgattggaatctcattgaatttacaggttacagtggggatagttgtcacctttacattggttgactgtcattgatatttctgttcaatacagttctatttttttcttttttctttttcttttttttttgagatggagtcttgcttttgttgtgatctcggctcactgcaacctccgcctccctgtttcgagtgattttgctgcgtcagcctccccagtagctgggattacaggcgcctgccaccatgcctggctgatttttgtatttttagtagagacagggtttcgccatgttggccatcctgaccttaggtgatctacccacctcagcttctcgaagtgctgggattataggcgtgagccactgcgcctggcccagttccatttttttcttgatagaagtcttcattagattttttttttttaggtttttatgatttttgttaccatcgtgaatggcaaatttttataaaaatactttttgttactggtagctacaaaccaattagtataagagacaaattgctaagaagcaagttgctgtttgtatcttgattttgtatctctcaatcttgctgaattcttagttgtttttgtttgtttgtttgaggtggtgtgtcttactctgacacctaggctggagtgcagtggtgtggtcactgctcaccacagccttgactgcctgggttcaagcaattctcccacctcagcttcccaagtagctgggaccacaggtgcatgtcaccacacccaaccaattttaaaattatttgtagagatgaggtcttgctatgttgcccaggctgctctcgaactcctgggattaagcgatcctcttgccttggcttcccagagtgctgagattacaggcatgagccattgcacctggcctaaactcttacttctaatgatagattctcttggatacctcatgcagataatcatataagtagtgaataaagatggtttttgttgctttctaagcctatgtctttatatttttcttctgttattgacctatgtaggaccactttactaaaattgcagcttttcctgggccttggctttatgtgtgtgttaatgtatggagtagaaaggggtatctcagaactgaagtctcccactaagggccaaaagtctcatttctagtccctacatatgagtcaaaggataaatataggttaagactagcaacccattgtagggaagaaacattgccaacttgtagcctaatatatatatttttaatatgttactgttttttccatgttactggcacacacttaaagatttctgtttcatggtggctaaaggattaatttaaaataattttttttttcctttttgagttgagtttcactctgttgcccaggctgaagtggagtggggtgatctcagctcactgcaacctctgtctcccggtttcaagcgattcttctgcctcagcctcctgagtagttgggactacaggtgtgtgccaccatacccagctaatttgtttttttttaaatttttagtagagacggggtttcaccatgtaggacagggtgatctcgaactcctgacctcaggtgatctgcctgtcttggcctcccaaagtgctaggattacagacgtgagtcaccacacccagcctaaaagaattattttgatatatcctttctgcatgaaatttctgtgggaaggcttttcaggtgatctgatctgatacagtgttaggactggaagtatagcttatactatataaatttccttaattgaaatttcacattatttaggaactctgtaccctgaagctgtctcactctggtatttaatgtcctacaattagtttaagagagcaagcttgggtaagtttcttaaaacttccttatatttgtttcttcatcaatattagtagtatagtatcatctcctatcttatagattattataagaaataagttaataatgtaaagcatttagaacagtattagttattggtctcaacctccttttagctgttatctacagcctttctggtagattttacttattctgggaagatagtggtgtattcctcataatctccttgtacattccaggtcctacaataattctcaaacattttagcatccttacatagactctccaatatcctggtcttatttccctgatctcctcaaatctactgatttttctcttcctcagacaagtgatttttcacttatttggtaaaatcctaattctggattaactcggttatcagattctttcattcctataccacctgaatgctgcagtaaaaagttacctcaaaggacatttaaaaaaaattataaattcatgattgttgatcacagaccctaatactgcatgggaatccactgcattatccttgtcagctagtttcctcatctttaccatgactgtttcaatcatctgttctcctcagacctccaacatgccctttctctttctacagaaaacttttcctgttactttccagtgaaaaccggagtacttggtgcaacccttagttgcccactatcttttctatattttctttcttcttaatgttaaaatagaggtagtcttgctttctatctaaggctttctccctttctctgcagtaccttatgctgttgatgatttcccttttttttgtatgtttaacttttctttacacttgattcttctcgtcagtatttaaacacgcttaattcctgctattaaaaaaataaaaacaataagcattaatgggtaccacattactgacctttctgttctctttcctagccaaatgctagaaattattgtttccatttctcacctttgactcacctgtttagtttattcattccactgtaatagtatttgcttatgataccaacaacctctctgttagtaaattaagtgaatatttttaaacttggtcttatttgatctctcacttggtgtgctagggcttctataaaataccacagactgggtggcttaaacaacagaaattaattttctcactatgtggaggctggatgtcaaagatcaaggtgttggcaggtttgttttcttctgaggcctctctccttggcttgcagagggtgccttcttgctgtgtcctcacttggtttttcctctgtttgcatgcatttctggtgtctgtgtccagattggattagggcctggaagatgaatttactcacctctctaaaggccctatatccaaatacagtcgtgttctgacgtactgggggttagggcttcaacacgtgaattttgggggagacagtttggcacataatctcctagcaggtttaaacagttttgaaaaactatccttggctggacgcattggcttacacctgtaatcccaacactttgggaggccgaggcgggtggatcacctgaggtcaggagttcgagaccagcctgaccaacatggtgaaaccctgtctctgctaaagatacaaaaataagccaggtttggtggcacatgtctgtaatcgcagctactcgggaggctgaggcacgagaatcgcttgaacctgggaggcagagtttgcagtgagccaagattgcgccactgcactccagcctgggcaacaaagcgagagtctgtctctggaaaaaaaaacaaaaaacaaaaaaactatcctttaaactctttggttttcattttgcctctctgatctattgctcttcagcctttttttaacaggcttctcttctgcttggttattaaaagtagaaatttcttgagtcttggtcctttggtattttctgcttctcttacttctgttatttctccccaggcagtcttaccactttgatttccatttatgaaggaactgcatacctgttgttccagcacttaatttgcctgagtttcagacctatttaataatgccatttagcttatcctagaggtaccttaaaaaaacgagttcaaatctgaactcataataatccccatccatgtgatttgaccctaatctacctccttagccttactattctttgttactctttgccatagctgcactatagcaaagcacggcctcttggtccaggcgtaatataccttttcctccagagtctttgctcttatttcctatgttaatatgctcctcatttttcatttacaacattacccagttttcaatacatacaaaagataaaaacattagttattagggaaatgcagacttaaaccataaaataatcactaaaaccaactagaaggggtaaaattaaaaagactgtcaactacaaatattctaggaagccatccagaacactcgtaaattgctggtgtgagtataaaatgggagagccactttggaataccactggcaatttcttataaatttaagcatacacctttggatccagcaattctacttctaggtgttttcggaaaaggaatgaaaacatgtccacaaaaaacttttaaaaattatatttacagtcgatttactccttacagctgacaactagaagcagcccaaatgtcaataggattaatgactaaaaattttttggtatattcatacaatggaatgccactcagtaataaaagaactacagaatggcaaatgtacacaacatgacatggatgaatctgacatacttggcaaaggaagctggacatgatactgtattattccatttatattaagttctagaagaggcaaagctactctggagtgaaaaaataaaaacagaacaaaggttgccttttgcttctggaaaatggtgatattcaactgggagggtgcataaggggccttcttgaagtgatgaaaatgttccatatctcatagaggtgtgtgttacatgggcttatctattaaagttgtacagctaagaagtatgtatttcaatggcatacatgtatcttagataaaataaacaagttttatctaaaaattatagaaataattgagttaggtgacagggagtgggaggtgtagatgaaagaagaattgcagaacgttggtggttgctgaagttgagtaatggatgtggggaagtttagtatactattttgtttgttttttgtgtgttcaaagttttccatataaagatctttttaaatcaaagttacgaagacacttaaacagcacctgtggtctgactgccagcttaataaagaatgcttactaatgtaattttcttctctgtgaatcgtaccctgattcagcactcctttctccccaccagccactatcctgaattcctcttgactttttaagacatcttccaaatatcattctaaacgtcacgtccatgggggaaagccttttctgattcagaacactcactagttttaagtgcccctgttaagtcctttcatagcttttatcttaattatgatttatttgcttaactattatcaacctctacactagactaagcctaagtttttggagggaaagatatatctattcttgctgttttaatgtctagaataataatacatttattgaatgattatgttggtgtagttttttaaaaaaattgaaggatacccaaattgaagagctattctattactgcttatgttgtaaggtcagtatgttataaaatgtatgaatgtgagagaatgactgagaaatgataatggtttctttctgattttattggcatctgataaaaacaaggggcaataaaactgtgttattagatctggaactatatccggaaagtcctgtcagttggttctcaaatgcccttgagctttattatgcttcttccttattaatgtattaagccgtgtgagtgaaagacccctttcctctttacaaagaatattctaaatcacctcttttttctctcttctctaatgtgcttcttcctttgactattagttttggtatttatcagggacagcgatctttaagagcttgttgttttgttattttttgtactgcttgctgtatgttttaaattttgatatatcttactgccaaatgaaggtgtgagctcataagtaataaagatagaaaaaatctgctttgcttataattataccattaaacatgttgtccttaatgcagatacacactattggtattgtaggtgcaggattatgttttgatttttgccagttcatatttccatggagtatttaggcaattttaacaacatagatttctgtaatgaatgtttttttaacttatgctgctgcttgagagacagagtcagactgccagggtttgagtcctatgcccaatatttcctagctctgtgacttttgagttatctgctttttgcctgtttcttcctcattaaaacagagatactaagtgtaccttatagtgttgttgggagggctatttgaggtagtatatgtaatgcctggtacttattaaatgttagatcttttttactttcttgggttctagggagtccaataagtaacaaatttttcctctaaggtttttttctttattggtggcaaaatatacatagtataaaatttacatttcaaccatttttaaatatacaattcagtggcatcaagtacattcacaatattttgcaaccatccatctctgtccatttccagaagtttttcatcatcgcaaataaaaacttgatacccattaaacaataactcccatctttcctcccctgatttcctggtaacgtatgttatactctgtctttatgattatgactattctaggtacctcataaatagaatcatacagtgtttgtcttattgtcctctcatttcatttggcataatattttcaaagttaattcatgttgtagcgacttctagttccattttttgtggttgaagaaagtacttccctaatagaaatgaaaacatgtttgcaaggctttttttaaagaatgcttatggatttagtcataataggtaacaactgaaaacattggtatggctgggtcaagttggactaattcactgtagcccagggtcatgtaagaacatggagttttcaaggtaggcatgggggtggtattgaggggagggtagtaagcataaaagacagtgctcagaagaagataagggtgccaggtttcagttccagggatgttcacagtaaggattacagtgggctaaattttaagattaattttataaattctaaacacatcctgttgactttaactgcaactcttaaaatgtatcaataacatgactttataattcctggaaactgatattttcccaatactttgaagaggtttaaaaatagttgactaatattagaaccaatatttcatgtattgatttttctctttaagtgacagttagtttagaaaatgctaaacttaagttagcagtatagagtaggtatatagttatactgagtatgaatgtcttggtaggcttggaagcctaccatttatgtcctcttgaggtacctgaattaccaaaagctttatgtattctgaagttattgaaaataagagcttttgggaattcaggtagttcaggagtgacttttctaaaaaacagaactgagcaccatacactacttcttatagccttttaattactgccaattgcctagaataaagttcaaattcgagggcaacacaaacatggccctttgtgaactgacagacatttcctaataattccttacatacatgatacccttgagccttacagaattaagaatttctgaatttaccttggtgtttgatgcatctgtgtttctatacgtgctgtttatttacctaaaatagactattctctatttagcctcaaaagtctcatggaaggttttaaaaaatcctgtctaagctctgttaattatattctttatactattatatttagtagttaatattataaactaccttacagtaatttttacagatacctggcttactccctaaattgagttgcttgagggcaggaattgtgtattaatctttgctgtattttctttgccttgcttactgtagttgattggtgctaattgaataaatgaacatttatgcttttctttttgagaatattatatgggtagttcctttagttgtttaatagaagatagcacagacttctatacagattggtttttatgggaaaacacaagagaataattaattaaaacataatttcatctagaaatttctgcagttggagcgaatattttttaagttattaacttacctagagtggaagattgaatttttgaatgatagtgttttagttattaaagtaaatggcatgtaagagaacttttaagttcatgtggcaaaacagggaaaaattgcaaagttagagtctgtaaggtttctgatgtttcatagactaacaaaaggttgctttattagcagcattaatgaacgaatcgctttattggcctaggcatactggtagcattgtcgttccaaagagctgagagttcttatttcaattcatgattatgttagacaagtgaagtgaaagccgttgaccttctcttaaggagaaaatagaaagtttttacaaatagattatagattaaagccttttaaggaacatgatgaagaactaaattactgctaaatcacttgaaaaatttatatttttttatgcaccagtcttataacagtaggttttttatagacctattaattgatatttaaaataataactttaataggatgattttgtagagttagtcaaagtgattgacaaaagcaaattattaaaatagtcaaactttcaattctccaaagaatgttcagttttaaaagcaatatgagatacaggaagtattaaactggagatgtaatgccacttggatggcatacttttaacattattttttatttgtttaaatggaatgcattttgcttattagaatcattttggaagtgccaatttgcttctgttaatttacattaaagattaaaataggaaccactggtgaccttctaaatcaaagttcttcaaaacggttgttttaaacagcaagaattacagaactaagtattgtatagctattaaaactgaaaattatgtagcagcagtccattttaaatttaacaattttaaatgatacttcaaaatataaaaaatacaaggaagaatataagatgcttatgtatctgtactgccaaaatttaacaaatgttaactttttgctatcactgtttcagaactttttaaaataaattaaaaatataattgaagattactaatctcattcttttgcctctcttctcagaggtaattacttttctaaaggtatcctgtaagttttcaatactttttaccacatatttgtataaaaagataaacagtactgatttccttttaaaatgtgaataataatatactgttggcataactttttaacttgcttttttcactcagtgtttatttacatcaagatttattcatgttgatatttgtatgtacttaaagtttgtttattttatcctctgaattattagtttggtccattatttatgatgtttagtttgattacttggttaaggtggtgtgacctgatttctctattgtaaaagtatccttttccccttgataattaatcattaatctgtggggtaatgccttgagactgagtaaacattgtttctcaacagcctttcacccagtgtttttaacatccattattgatttatgcctgaattattggttacattgatgcttgcaaaatgattttctacatctattattctttccacatttattagctggcattcttctttctgtaaaggagtttctgtcctttcactaagggctcatggtttcttcattaattcggtgaactaaaatccattattgttatgtagtcatgcactgcatatacgacattgatcccatgagattataatggagctgaaaaatttctatcgcatagtgacgttgtagctgttataacatgttaggactaacgtcacaaccaacatattacatgtttgtagtgatgttagtgtaagtcaaatctactgtgctgccagtctaaaagtatagcacatataattgtgtacagtacataatacttgataataataataaacagctatgttaacggtttatgtatttactatgctatactttttgttgttattttgaatgtacttctacttattaaaaaaaaaagttaactgcaaaacagcctcaggcagttccttcaggacgtactccagagacattgttattataggagatgacagctctgtgcatgttattgtccctgaagaccttccagtgagacagtatgtggaggtggaagacagtgatactgatgatcctgaccctgtgtagggctagtctaatgtgtttgtttgtgtcttaggttttagcaaaacagttttaaaagtaaaacaggaattttttaaaatagaaaaggcttgtaaaataaagataaaaggaaagaaaatatttttgtgcagctgtacaatatgttagttttacgctaaatgttattacaaaagtcagaaaagtttaaaaactaaaaagtttatatgagctaaggtttattattgaagaaagaaaatgtttaaaaatagatgtagtgtggtttgagtgtatatttacaaagcctacagccgtgtgcagtaacgacctaggccttcacattcactcttcactcaatcattaactcacccagagcagcttccagtcctgtaagctccattcatagtaagtgccctatataggcatatgattttaaaaaatcttttatactgtatttttactgtaccttttctatgtctagatattctcagacgcaccatgttacagttaccaggcagtattcagtacagtaatatggtatacaggtttgtagcctaggagcaatgggctataccgtgtaggcttgtgtaaatacactctgtgatgttagcacagtgagggaatagcctaacaaacacatttctcagaagatatccctgtggttatgtgacgcatgactgtgtatcctttggtgactcaaattgttaaaatttagccagcgagaactcaatcagtctggtttctgtttctttgacatgccccctcatttttatagcaccagtttgctttgtggcacaacaaaacattctctccttaccttatggttttcttaccccagacctggacttggctattttaccaaaagttcttgtttctcttagtggggaatggtatttggaagccaacatcatggatgcattactgggtgtcattgcttctattggttcttttcattagacagaataagaaaatatacttttaaaaactagtgatatttgaattcctagtgacaccaaggttcttttctttccttgtttcatattcctcttccataatgaagatccagtttaccaataacatcaatatatgtttattcatttgctctgtcttatacatgatataacattggaattactatagcaataccactactaataataatctcaataaagtttaagatttctatgcagttttttatttatagcgtatagcccagtgaaggtatatagtcagatcgctatgcttatagttattttaattatatttttcacataggcaatttgaaactttttctaataggaaggttaagcctatttacttatcttgacataactagtatgtttaatctaactgatatgttgtatttactgttgttttatgttatatatgtttttcttttactcttttttaaaaagttatttttactaatacaaaaatcttcctttctctagatatttagaatgttggtcttagctcttatagtttcccagtctttttggtatttttgttgtcattactatgtttaattttgttaactagaattcatgttttcttttcttcatccatttaattgggctttatactttttttttttttttttgagacagagtctcgctctgtcacccacactagagtgcagtggcgcgatctcagctcactgcagcttctgtctcccaggttcaagtgattctcctgcctcagcttcccaagtagctgggattacaggtgcttgccaccatggctggttaatttttgtatttttagtagagacagggtttcgccatgttggccaggctggtctcgaactcctgacctcaggtgatccacccaccttggcctcccaaagtgctgggattacaggcatgagctaccgtacccattctatatattcctttgcataaatgttgtgttctgaggaaaatttgtgtctgagcatttaataatttaaaaaattaccatgtgagtggatttattttatatgaataatgtgccacatgtaagttgttctgcagttgattttccctttttgtggagcttttacaatattgttagtatgtttacttgtccctttttccccttgtatgtgtagccaagtaattataaaaaatggtgcactttgccatatctgagcttctctttttcccctcaagtagatactgtgcatttgaactgcaaaaagggtcatattcttgctttatctataatctcttttaaaggcaaaggctgatggttactttttctttaataagaatcttgatttttcattttgtatatttgatatttatcattttaagaaaaaaaaatcctctatacatagttgttagaaatttatgtagaatggatgttggaacccaatcataaggctgtaaaaccataaaataagtagtaataaattttaaaaaatgaataggatttgtatgaagaaaataacaacacttcattctaggatcaaacaaatgaagagagagctatttttcagaatgaaaaaccaaatagtgttgcatgtagactctattcaaagttctagtctgtaaaattaatagcaattctagtcaaaatagctgttttatcttttgagaagttcgggaagtttgacaatctgattgtcaaatatatgttgtagagtatgttaatgaaactatccagtgtaaaatttaaaaataaagaccagtattgttatattgtactgtattgcttgctatactgtagtggtgtagtaattaaaacaccattctagtgatacaggaatagggtccaaataaataaaaactgagaaggaaattgtatacacatgcaagtgtgtgtgtgcacacacacacacaacacagaaagggtttttttttgttttttgtttttgttttttggagacagggtctcactttgttgcccaggctggagtgcagtggcatgatcatggctcattgcagccttgacctcccaggctcaagcaatcttcccaccttagccttgtgagtagctgggactatgtgtgccactgtttccaactaatttttgtactttttgtagagactgggtttttcatgttccctaggctggtcttggactcctgggctcaagtgatccttcacctcagcctcccaaagagctgggattacaggtgttagccaccacgcccagcctcaaaaagctttttttttttttccctataaagagccatatatctaaaaagggaaacagatttgactctattacaatggaattctgtgagagaaaacattttaacaagatcagactatctagtagaaaaatatttgtaacatatagcaaagttttaacaaacataataaagaccttctaaaaattaataaaattgaaaaaatgggttatgaataagtacaaataattcaaagagtgtacacaaattagttttcatagaaatatgcataaagatatatgtacaagtatgttaattttagcattatttataataccaaataataatattccaacaaaatagttggaataatctgaatgttcaccagtagggaaatggtacatctgtattttagaatattagcaagctgattaaaaagaataagatctttgtgaccaggtatggcaaaatatcccacattattcatgaaactatagaatgatctgtgtagtatgatctcatttacttagctcagtaggtctattgtttgtggtaaacttaaaattattttatttatcttataggatagggacttttaagttaacctattaatattattaggaactaaaatatatcactgtaagagaataaagacaaatatagaataaaaggaactaagaaaaatattttacatcagattaattggaaatattggtatgaatccatgagtaaataaagaaatatctctccccccaccctgttctgtctaccagaaaatataagaagcatggacaatctcagtagcaatggttacatgcatcttggttgctaaatatggctccccattgaaagaattcaagatgtcttagaaaaatagatgatcccagttcttaggaaaacacacagcaagccagaaaacaaggaagtggtgaaaaactgaggaattatgtaaaaaagaaccaactcaaaggagttctagtggccacctccgaaataatgattgcaatcacaggcatatcttggagatgttgtggattcggttccagaccatcacaataaaacaaatattacaataaagtgagtcacacacatttttgttttcctagtgcatataagttatgtttatacaatattgtagtctgttaagtgtgtaataacatgtttaaaaaatgtacatgccttagtttagaaatactttattgctaaaaattgctaataatgatcaactgagtttttagtgagttgttctaatctttttcctgctggagggtcttgactcgatgttgatgactgctcgccagtcagggtgatagttgctgaaggttggggtggctgtgacaatttttaaaaataagacagcaatgaagtttgttacatcaattattctttgatgaaatatttctctgtagaatgtgatgttgtttaatagtgttttacccacagtagaaattcttcaaaattggagggcagttctctcaaaccctgctgctgctttgtcaactaagtatgtaatattgtaaatcctttgttattatttcaatgatgttcacagcatcttcaccaaaagtggattccatctaaagaaatcactttctttgctcttttttataagaagcaacacctcagccattaaaattttattttgagatttcagcaattcaatcatgttatcaggctctgcttctagttctcttgctatttctatcacatctgcagtgatttcctccactgaagtcttgagctcctcacatccatgagggttgggatcaacttcttccagagttctgttaatgctggtgttttgatctccactcatgaataatgaatgtttttgaaggcgtccagattgatgaatccttcccagaaggctttcaatttactttgcccacatctatcagaggaattcctatctatgacagctatacctttatgaaatatatttcttaaataataagatttgaaagtcaaaagggcttctcaatccatggcctgctgaatggatgtggtggtagcaggcatgaaaacaacatgagtcttctggtatatctccatcagagctcttgggtgaccaggtgcaatgagcaatgtcagtgagcaatgagtagtattttgaacggaatcttttattctgagctacagtctcaacagtggacttaaaatattcggcaaaccatgttgttaaatagatgtgctttcatcaaggctctgttctgtttgtactgcacaagccagagtagatttagcttatttcttaagggccctacaatttttggaatgataagtgatcattgttccacttggaagtcaccagtagcattggctcctaacatgctgagtcagcctatcctttgaagtttggaagccaggtattgacttatcgtttctagctaggaaagtactacatggcttcttcttccagtagagagttatttcatctcaattgaaaatctgttgtttagtgtagccaccttcatcagtgatcttagttagatcttctagataacttgctgtaacttctccatcagcacttgctgcttcatcttgtacttttatggagacagcttctttccttaaacctcatgaaataacctctgctagcttcaaacttttcttctgaagcttcttcacctctctcagccttcatagaattgaagagaggactttgcactgaattaggctttggcttaagggaatattgtggctggtttgatcttctgtgcagaacaccaaacctttgtccatattagcagtaaggctctttcttgttcctattattcatgtgttcactggagtagcacatttaatttccttcaagaacttttcttttccttttttaacttggttaactggcacaagaatcctagcttttggcctatcttggccttcatcattccttcctcactaagcttaattatttccaacttttgatttaaagtgagtaatgtgtgactcttcttttcactccaacacttagaggccattgtggggttattaattggacaaatttcaatgttgttttgtcttagggaatagggaggtctgaggagagggagagagatagggattggctggtggttggagcagtcagaacacatgcaacatttatcagttaagcttgccatcttctgtgtttgtgatttgtggtggcccaaagcagttaaaatagtaacatcaaagatcacagatcaccataacagatacaataataatgataaagtttgaaatattgtgagaattactaaaatgtgatactgagacacaaagtgagcacatgctatttgaaaaattgcaccaaatagacttgcccaatgcagggttgcacaaaccattattttgtaaaatccacaatatgtttgaagtccaataaaatgaaatgcagtacaaggaggtatacctataattgtaaaacctttgattgaaaaaaataatccatgagtccagagagataagaagaaaaggaaggccctcctttagagaattgtagtagcagtgttagaactggaaaactgtactgtaaactccaaagaaacaatctgtgccaacagcatcaatggatgccaaaacctttaggtgtaagagtgctgtgaacatgatacgtatgcagtggtgcccaattattctgtaaatcttgtacgactacaaagggaaaatgtgcctttgcaatagatctatagattgccacttgaactaagtggtcacacttataatgggacagcctgataccatgttccttttgttatgatataatgcggaatgatgtaaccttgggtgtattcttgcctaatgtttatctacaatccagtcaggtttctagacttgatcttgtttccaagaaatacaggaagttgtggagtaagtcagaatacattctgggaagcagtcagacaaatctaggatgtgggacattctgtaagaaaactggccatgagtcctgacacatcattgtcttaagaaaaagatatgtgtgtgtgcgagagactattctaggacaggcgagaccaaagagatttaacaggcaaatgttatacataaattttcatttgcccttggattgctctctcaaccctgaaggcttaaaaatagatatctttgagacacttgagaaaacatcaagatactagcttaccttatttttacttttttttgactagatgtcttaatggttttattactttgtaagacttgttttaaaagatatgtttcactactcagtatagtctatgataaaatcagtaaatataaaatatttgtcatttgcatctcttaggaggttcgtctccattgatgaaagctcagcttatgtgtaactgtcttccaaagtagcttgcttggaaggacattcactatcatcatattctgctttatttcatagctgtgtaatcacatctactacatccttgtttatcgtttattagattactgcttttaagcctattttagttctttaatctgagaataacagttgactcagttaatttcattaaagcaagattgcctgtaatttttttataaccttagaaaagtactatgcccaacagcaaaacaaaaacctaattcaaaaatgggtaaaggacttgaacagatatttctccaaagaagatataaaatggccaataaacacatatgaagagtctcaacaccagtagtcattagagaaatgcaaatcaaaaccacatttagatacaatttggctattagcaaagacacagaaaatagcaaggattgggaaggatgtggagaaaattggaaccatgcctgcactactggtgggaacgtaaaatggtacagctgctatgaaagacagtttaccagttcttcaaaaagtttgatgtagaatgattgtatcatccaacaatttcactgttaggcttatctccaaaagaattgaaagaagggacacaaactggataacttatacagcagtgttcatagcagcattactcatactaaataaaagatgaaaaaggccaaatgtctgtgaacagatgaatggacaaatgtgtttatacatatagtagaatattattcagccataaatacaaatgaaattttggtacatggtacaacatagataaaccttgaaaacattgtgctaagtgaaataagctagacagaaaatattgtatgattctacttagatgagataccccaagctggcaaatatataaagctggcaagtataatagaagttactaaggcctagggtgagggaggaatgagaagtttgcttaatgagtatagagtttctatttggaatgatgaaaagttctggaaatgaatagtggtgataattgtacaacattctgaatgtactcaatgctactgaattgtccatttaaaaatggctaaaatagtacattttatgtatattttatgatacaaaaatatttttaaagtaaaagtttgtattttgatgttacttgtgaaaaatattttcactaataaaattttgttgtttcattgctgcctctttgaaaatgtattaaaaattatatgcattttgaaatattacagctatataaatgaaaaagagtatcagtaaagaattttagctcaatactctcatatcatgttatttattgtcctttctgtttcacattaagatatacagagtcaacaatttaaatttgcagattaaaaaacactttctgcttttggccctatagaaataaaccaaactggaaaaggtgtcatgtttcctagattgtggacacagaaaaagaagagctcaaaaggacaagtaactcagcacagtagatggttattgacctgatcatatttaagaatatcctaaaaatgcaaaaatgttaagaagcaaaactgggaaatactgggatgatatatgaaaaattcaaatgacaaatatgcagtggttgagtatttatgggtttttttggggtttactattttttcccccaaatttaatgtctcctcttttaaatacttctaccggattctctttagatgatgctgcaattggattgagacattttcattgtttggctttgtaagatttccaaatgcgtatagttgttaaaatacgtattcaatgagggtggaaggaaacacacaaaaatagaagtatgtcactctggtaagagatgataggtccgatgactgctaatacttttccaaatggccttatttggatccttcttagaacttttatatttttctaatattgacctacccctttggaactagagtcccattgtcaagaactgtgaagaatcttgacattttaccctacttgcaagctagcagttactcagttgtagttttatggatggtggttaaagatatgagactcctgagttggagttgaaggacagtttattagtgcagcaagcagtagcagtgaccagagtattagcttcctttttttttttttgccgcttttccaagccctggttcccatatagtagtgtgaagaggagggctacatgatgcttgtacacactgtggattgcataacagatgcggaatcctcagcttcttacagaatctgaatagtagtgtataataggaagtaagaatgcctttctttgctttggagcaagatactgtctttgtctcccaacactattgctgtaaaagtatcctcaaaaggatagtgcagaccaaaagggtagttgagtgtcttgctcctaagaggtgcagaaacatgagagtctcatagaaaattgtctcccaataatattttctgctcatttctatactattttgtcttaaggcaaatttttttcatgagtataccactctgatttatctgacagaagatagtgaagtatctgttcaaatagttagctggcttttaaaatcaggttatttgctttcttattgttgagtttgagagttctttcttgattctgggtacagtcctttgtcagatgtgtgatttgcaagtattttctctgagtctgtgacctgtcttttcatattcttcaatctcttttacagaacagaagtttctaattttgattactatcaggtttttctcttatgaattgtcttaggaactttttgcctagttgaaaatcacagagtttttttctttgttttgtccagatgttttattattttgtgtcttacatttagatctgtgatctgtcttgcattaccttttgtttgcataagttatgaggtagagttcaggttcattgtttttgcgtatggatatttacttgttctagcactatttgttgaagagactatcttttccttcggtgaactgcctttttacctttgtcaaaaatcaattgcccttatttgtaagtatattttgctggatttattattctgttttgttgatttctgtgtgtgtcctcttgctaatacatcattgccttaaatagtaacagttttatggtagttattgaaattagtgttctcaaattttgttcttttgctgaattgttttggatattatagtacctttgcattttctttataaattaggaatcagcttgttgatagctacaaagatactttgagattttgattggtgttatgtttaatgtacatgtcagtctgaggagactgacattttgccaatattgagccttccaatccatgaatatagttatttttccattttttaggtctttgattattttcatcagtgctttatagttttcagcatgcagatcctgcacatattttgtggaagtgtatcttttccagatgctattgtgagtgctactgctttaaaaatttttgatgtactaacttattgctgatatataaaaatatggtcgatttttacatattgattttatattccacaacttgttaaaatcataatcataaaaccctttggattttctatatagacaacatgcttttgaataaaggctgttttacaaaatttcattttaaatttgtataccttttatttcatttcctttcttaattgcacttgctagagctttccaggacaatgctgaatagaggtggtaaaaacacatctttgctttgttcctggtgttttattactcttttattattagtgttatgtttagagatggggtcttgctcagttgcgaaggctggagtgcaccggtatgatcaaagctcactgcagcttcaaactgttgggcccaagtggtcttcctgccccgccttcctgagtagctgggattacaggcatgtgccatcatgcctggctaagttttttattttttgtggagatgaggtcttgctgtgttcacaggctggtcttgaactccagtctcaagtgatcctcccacttgtactgggattacaggcattagccaccacacttggcctcttattatcattattattattaatatatataaataaatatacatatatttatttattgagacagagtcttgctctgtcagccaggatggagtgcagtgccacaatctctgctcactcaacctctgcttcctgggttcaagtgattctcccacttcagcctcctgagtagctgggattactggcgtttgctaccatgccgggcgtggtggctcacgcctgtaataccagcactttgggaggccaagacgggcggatcatgaggtcgggagatcgagaccatcctggctaacatggtgaaaccccgtctctactgaaaatacaaaaattagctgggagtggtggcgtgcgcctgtagtcccagctacttgagaggctgaggcaggagaatcacttgaacctgggaggtggaggttgcagtgagccaagattatactattgcactccagcctggcaacagtgagactccatcttaaaaaaatgttatgggatgaaattttaaaaagacctttgaaagatacaagataataggattcaattctgtataaggtagagataaactgttgatttgtactagtttgatttgtgaaatattacaattaggctaaatcaaataggtgatgggtaatttgagaattgatttctaatgctttgtcctagatcattcaaaatctgatgttcacataactacttagaactcttgactagttgtctgtgctttttaaaaacctttacctggctggctacaacttttctaccgccataattaaaattgaaaacaaaataattagtctcttaggaaaataatttacgaacactatctaaacatgctagatttaaaactaccagtatttacatggttagaattcatgagcagtcttcattagacttactgctgaatgaccagcatacagtcgctgaattcattgtgcatcttttaagggaagattgttttgcttgtatctttgcagtcttctctgagatctttaatgttactagaagttataaagtttggttaattttcctaataagcctttatctgattctgtgggctatttaggtttgggatgggatctgttgtcttaatttggtcaccattggctcttggcttcaaagtggaccctaaactatattcctagtctgtctagtatttgtcttttttttgctgagatatgtgacccctggggaggtttcccttattttcaataagttaatctttctcttttaggaagaaaagacaaatttctcataagggagggattttttccttttttgttctagagtgcctttcaaattaacaattttgttcatatcaaaagttgcccataatgatcactgtaagtctcaagttattcttggtgaaaatatgccatttagaaagataagtatctgaattggaagaagcatgtttcatgcttggagaggttgtggagttagactgacttaaaaagtcagttttgtgcagcctgggcaacataacaagacccagtttctatggaaaataaaaaaaaaattaggcaggcgtggtggtgcacacctgtagtcctagctactccagaagctgaggtggattgcttgagcccaggagtttgaggctgtggtgagctatggtaatgccactgcacttcagcctgggcaaaagagtaagacctcatcttttaaaatatatatacaggtttttcttcaaaagattccaaaggtactgctttaaaaaatgtaagattttctaagcaacagggccgagtactttgctttaaagtttttatgaatgcctggcttggctatctggttgcttttgaaatcaacatcttactaaactattaatgcttttaaaatgtgtgcagaattacatatttgtaaatttaacttgagtctgtctaggaaagcattagcaagacttttgtttttgtttttgtttttggagacagtcttgctctgtcgcccagcctggagtgcagtgcaggtcactgctcactgtagccttcacctcctgggctcatgctatcttcccacctcagcctcctgagtagctgggactacaggtgtacatcaccacacccagctaagtaaaaagatgtatttttcatagagacggggtctccctatgttgccaggctgggcattagcaagattttacgggaaattttttttatcattagtagaaaataataaggaatcccttgctggtaaaatttatattgaaactactcttaaacatatttatataaaatgaccagagatatttaatttacctttatataataaatagaaataaattgaaatacattattaggagattcagattttttaattagtaaattagtgtgatggcttaaagttattgagaggtaatttttcatctttgattagaaaaacttaaaaattgtataatttacatacacaaagtatatatgagctttttatattgatgaactttacaaaatgaacacacttttgtaaccacaacccagatcaagatatcagcttctcaggagcttccctgtatctgtcatattcctttacatgaaaggtaattaatattctgactttgtaattagagattagcttggcttttttttgaatttttataaatcataaagtatataagactatatgatttataagttttttcaaactggcttctttcccacagcagtatgtttttgagatggatatttggcttacagcatttttcattttcactgaggcttagggtttcattgtgtgaatatccttagtttatttctcccttctgctgatggacattcaaattatatcaattttgggttcttacaaatagtgctgctgtgataattctcgtacatatttttttgtgcatatatgtacacatttccattgagtgtatgctcaggggtagtatcacttagtctttgggtttatcttcagcttagtagacacttccgaacagtttcccatactgtttgtattatattattatacatttttacctgcagtattttagaatgtcagttgctacagatttttgcttacacttggtattgtctttttttagataaaagttagtcattttggagggtagtagtatgtctttgtgaagttttgtttggtctttagcctatcttaaaaatcggtttgttttttttgttgagttgaagtatttttaaagaatatattttgggtatgagtctttagtggaacatgtgcattgctgatattttctcctacactgtagcttgcattttcatttttaatttatttaaattttattttattttttaagagacagggtctcactctgtcacccaggctggagtgcagtggccttatcatagcttattgcggccttgaactcttgggctcaagcagtcctcctgcttcagccttctgagtagctgggactgcaggtatgtgccaccacacctgggtaatttttaaatttttttgtagagacagaatctcactatgttgcccacactaatctcggactcctggcctcaagcagtcctcctgcctgggtctcccaaaatgctgggattacaggtgtgagccactgcgccagtcatttccttctttccttctttctctctttccctctttccctctctctttctctcttcctttctctctccttccttccttccatccttccctccctccctccctccccattcccttccccttccctccccctttccctccctgcttccctccttccttccctccttccttctctctttccttccctccttccttccctttttccttccttccttccctccttccctccttccttctctccttccttccctccttccttccctccctgtaattgtaatgaaattgtttgtttgtaatgaaattcagtttttttatgtgctgtgtattaaatctatgcctaaatatcgtgacagtatttatcttgtcactatataagctttattatttaaaattttatatatataggtctctaatatatcttattttttttcccaccttttctgtggcttgtggatagatgttattcattttactgattttgattttaacagtagtagtcatagaaaatctattgatgtggcttactcatgagaaagcatttttatttttatatgtgatattataatttctagtatggcaacattttctgctagttatgatatgtgatttttactctgcagattctttaagaggaatttaaaaacagctttattcctgactttagagaaggcagtcagttttacttaatatgtcattttatgtcttatttgttatgttttgtttttgaggagtcatactggtagtttaaaaatactgagttttatcatcaaatgcagctacaatgtttaagcagatgacttaatctccgtgaggcatgaggtgattaaaaaatgtatgtatatgctttttcaaatacagagtaaattgcctggcagacaagaagagcttcataaatattatttagtgctttttattctagatttttgaaataaattggactcgacttgatgcaaaggatttgtactataagtttattataaaattattaaccgctagcttttgtaaatagtaaaattgatttgcccattatacatttctttttgttatgcataaaatattttaatttttaagtaccaacataggatggatttttaaatgcttcctcatgttaattttttaataaacatagaatgaatactgtgtgaaatattttcccactttattggctctgttttaactcagtagactctatagttagattgtctaggtgccacttaaaagatggtaactagataccagaatgtgttacattatatgttgattgtcaggaagaactgaatgcatgaatgcttccctaagacattgttagcatgccgtggtcttgatctataattttttgtctaagttgtgatagagctcattctttacatggtctagtaaattcagccaatgtctttataattcattagcatctctatatattacctataaaatttaaattataaggctatgaaaaaattgtttcatatgtataatataccaaagttttagcatatggtttggattttaaactgttttactgaaacaaattaccgtcattaaatggaattcttaggtagacattacagagtaggtgtaactctgaagttgaaaatggtttcatcttcttttttaaaaaattttattatatagggaatctcaaccaccagcaggattttaaaaaggctatctaaatatttggtaagtaaatattctccatgttaaatattttgcgtgttacttaagtttacacatatgctacatgaatgacatttacaatagtgagtgtaaattatagttataattaatattttaaattttacttggttactagaatttttgtctccttaaacctattccaatccttttctctcttgttctttatgttgtgtccagagattctttcagtgtacagccttattattaagagttcaaggaaagaaatggttttagtgagcaggaatgctaatactttatgaataaaacaggatttcccctacttgagattttgtagtatttcttttttgtgaaaacactaatacagatttgttctttagccatccattgcttgactcctccttgtatttaggtcctgtttgctgcaacatgccaacatgaaccaaatactgtggtcagggtctatgcatttcctgtttagattattctttgttccaagcttaaaatatgaaaaattttgtaggtgtctgtagttaagaatttcataaacttatgatttcatagataaagcttttacttttttatttttgagtaaaggtaggtgagtcaatctcagaatgtattttacttcaactacatagtattttatacctgtgtactatggtgattatatagatataaaatgatttggttaatcataatagattttaaagttattaaatatgcttcagaatgggaaagaagtgctgagacattgcataatgaggtgtcatagtataggcttttggttgtttttaatatagtcaatagcagattattaaggaggaaatatttagtgataagcattggcttcttttattatactttttaatgtgtttgaaacagcaaatatttgaccaaattatttttcttaaattttatagtaactacagtaggtgcctaaatgttcaacagttatactgttctgtttactgtaaatttgtaataaatataaatacttttaggagcaaatgatgccacagagatctcttaaacagatttttactactttagaaggtggtaactgatttctggagttgtattaagtacaaaagagtaacttattgtcatttttttccctattgatgcgtatatttgagttagagggaagcagtcgtctttgtgttatatgcttatcatgtatatttcatcttagcaaatttattaattttagtgacagttttattgagatgaaatttatataccaggagatttcatctctaagtgtacaattcagtgatattttaatatattcaccaagttatgcaactaccagtactttgtaatcttaggttactttcatcatccttcttctggacgccctctatcacccagccctaggcaaccactaatacactttctgtttgtataggtttgcctattctgggcattttataaaaacagaatcataaaatatttgtccttttgtgtctggcttctttaatttagcatgatatttaaagcttcactcatgttgtagcatgtatcagtgtttcattcatttttgtggctgaatatttctttgtatatcatttgtgtattcatcagttaatgagttgtatccacttctttttttggctattaggagcgcttttctgtgaacattcttatataagttttgtgtagatacgtttttgagtatatatggatggggtgggattgctcgataatatagtacctgtgtgtaactctttgaggaactctcaaaatggctgtaccattttacaataccaccaacaatgtatgaggattgtaatttctccacgacctcataaacacttattgtcagtcttttgtatttcagccattctagtaggtatgaagttgtatcccattgtgattttgatttggataatgactaatgataagcatcatttcacatgcttattgaccatatatcttctttggagaaatatctgttcaactcctttgcccattaaaaaaaaaaagattatctttttaatgttgaatattgtaaacattctttatatattctggaaactagtcccttatcacatacatgatttgcaaatattttctcctatggattatctctttacttttatgatggtgtcctttgaagcagaaaagttttaattttgaagttcagctgatctgttttttgttgcttgttcttttggtgttatattgcagatttgtttttaaataaatactcactgatagcctgggtctatgacaaagttttaatcgtcataggactacgaaaaagcattggcatttcttgtgtttgacagtcaaagcccaaaggaggcaattatccttgtatttgtgcttcagttcctcagctaattgttttaagcaaaaccttacgttttgatcccatgtgttactgtgaaaatattagctgtgtgtgatatgtttattgatattagtcttgtgaatttatgtttagttttttttggattgaagaagggaggggccatccttaatagatgttattagtccgttctcacgctgctgataaagacatatacccaagactgggtagtttataaaggaaagaggcttaattgactcacagttcctcagggctggagaggcctcaggaatcttacagttgtggcagaagaggaaccaaacacatggtgacagcaaggagaagtgcagagtgaatcgggggaaaagccccttataaaaccatcagatcttgtgggaactcagtcactattatgagaacagcatggaggtcatcatccccatgattcgattaccttgcactgggtcccaccacaacacttggggattgtgagaactgcaattcaagatgagatttgggtttggacacagccaaaccatatcaatagtttattatgacaattactgcatattttatttcatagtttaagaaaccattgctttttagactaaggattatcaggttcttatctttcttgtgaagaaggaatagattttgataatctcactgattttaatcttgtacagattattattattattattttgagacggaatctcgctctttcgctcaggccagagtgcagtggcgcgatctcagctcactgccagctctgcctcctgggttcacgccattctcctgcctcagcctcccgagtagctgggactacaggtgcccgccaccgcgcctggctaattttttgtatttttagtagagatggcatgtcaccatgttagccaggatggtctcgatctcctgacctcgtgatccgcccacctcggcctcccaaagtgctgggattacaggtgtgagccaccacgcccaacccagattatttttaagcaatgatattttggtgtttttgtaaacttatgtatggaatatgtaagtttttcctctattaagtaaatgtctttaatttttttttttttttgaaatggagtctcgctctgttgcccaggctggagggcagtggcacaatcttggctcactgcaacctctgcctcccgggttcaagtgattctcatgcctgagcctcccgagtagctgcgattacaggcatgtgccaccaaacctgactaatttttgtatttttagaagagacggggtttcaccatgttggtcaggctggtctcaaactcctgacctcaggtgaccacctgcctctgcctcccaaagtgctgggattacaggcttgagccactgcgcccagccagtgtctttaatttttaaggtaccaatttagttccttattttttatcgttatgtgacgttatgtgatcagttgaaggtgtatttcatgtattttgacctataatgttgtctagtttagtttctggagaaatggtgtgtaacagtggaatgtcacttgtcttcaaagcattagctttctgtgtaaggttgaggttgtaatataaatgcgctagatgtgtttttattaggggaattgactattcttacctttcatgttatttgacagtattctttgtcaacttcaaataacggcctattttgatgagattttatagtgctaggatcactgatattcttgttagttgatttaaaatcatggtgttcattggctaacagtcacttgagtggctattagatgggtaatattgttctcttttttttttttttttttgagaaagagtctcactctgtcgcccaggctggagtgcagtggcatgatcttggctcactgcaacctctgcctcctgggttcaagcgattctcctgtgtcagcctcctgagtagctgggattataggcgcatgccatcatgcccggctaatttttgtatttttagtagagatggggtctcaccatgttggacaggctggtctctaactcctgacctcaggtatccacccactttggcctcccaaagtgctgggattgcaggcatgagccaccgcgcctggccagatgggtaatattgttctatacattgatattcttatggtttatgttatagtattcatggaaatttagcaatggaaaaggaaatgaaaaacttattaatgatgttgttaaattttcaattgtgatgacagtaaggaaaagacataattgagagctacctccaattgtttatatcaaatgtggtaattaagaggattttggtgacctatttatggctgaattaaggcaaaatagcttttatatccgattctttcctcttcccttctcttgttctgtgtgcttactatcataataattgagtatacattaattttctttaagtatttatattttaaataaggttttgatgtcagtagagacttttttttttccttgaaggtaagtaccctttaaaccactaatttgtcactagagtctatagtcgcttccatatataataggtagcacagcttctcaggtcatgtcctgctttgctgctctgctgaccaataatacaccttggagatactggttctggttatatgaatagaacaattaaattgttcttcattagatggatctttgattactaatcttactcagaaaggctcaactggacttggattattctaattgccaatactgtttttgcacaaatgtctgcaaatcattgaacagtaatactgacccctttttcttccaataaagttattgcagtaatcacatcattgcagagaatatttcttaaaagcttttttcaatatttacttctttaatgaaaaacttaatccagattaagccacatacatagacgtcttgactttattattttgaatacctgagatttccaaaatctctttaactgtcttcacacaatttctagtctgtcattttaattaattacaactgttcatttcttctgcttttgtttcctcttcatgtgctgcctagcgttgctagagtttcatcagtttcctgccttgagaggtttctagtacaaattaacattttcccttcctagtttatgcaggttccttcctggtaatttttatctttttgatctgtttcattctgcatgttagttatttggaaccattctactgtgctcaaatgaacagtcctatccctagttctcttattcctttttcatttcatattttataccctttttcctcagtctgtcttaaagaataaagtgttttcatttcaaaagcttattatctctgtttttaatcttagcccttctttcctctactatttttcttcctcttaatgctccatgctgggttctctgccttatcagacttaatcttgaaaagtacgttacttccttcccattatctccaaggttgttatagtccattctaggtgtctccaatttctcaactcttactcaattactttctgaagtttgactattatttctactagtgttcagctttcttaagtgtaagtaaacacatttcttacttaactaggtagataaacattagctggacggatctggccgtaaacatgctattatttccaagataaaactgagttccaaatggctaaatctttgtcttttcacttttcatccattcctgtcttttttagcatttgtttttttgctgcttaacattcacttctcctttgtcttctgtaataaaccttaatggtttttctcctctctctggctaaatgtttgttttctttgttactgctttcttcttttatataccacttatatataaattgggttgaagctttgctcttagctctttttcattacacatcattttgaggagatggtctcgtgttcattatgtagattatcacaaatttgatgactcagaaataccacatttttcttagtgacctgttttaccactttctcagtcgccaggtccttagcaccccaaaattgttatttcccagtggaaagtttgtcttgtacaatgcttgctatatggttggaactaagtaaatgctttttgaatcatgaactaactccttatctgtctttttttttcttaagataatggttttaactgtccacctattctcatgtcacaagccctgtaatatctctgaccacttcttgtctacacttgttaatattagttgtcatattttggtcctaagtttgactaatgaggaagatgacatttttgtctgcagatggtggaaataaaaatcacagagattgtgatttccttatggttttgtgggtaatggtggagtttaaacttacaacgaagtttctggtgacatgtttcctagttttcacagaaaacattctttttttttttgagacagaaagtctcactgtgtcgcccaggctggagtgcagtggtgtgatgtgtcggctcactgcaacctctgcctcctggattcaagtgattctcatgcgtcagcctccaagtagctgggattacaggcgcccaccaccacgcccagctaatttttgtatttttagtatagatggagtttcaccatgttgaccaggctggtcttgaactcctggcctcaagtgacccacctgtctcagcctcccaaagtgctgggattacaggcataagccaccacacccagccaaaaacattctttataatgatacaagtaatataccagacaagaaattactcagtgtactatgtgagattaaaaaaaaaaaaaaaaaaagactggaagtggaagtgggagtacttcaaattcttgtctgatgaggtaaagattttaacttggtggaaggaatagaaatcaaggatgtgagaaatttttcattaattagaatttcgagattcttcttttaagaatgcagtgatgctagtgaagtggttgagtgaaccatctttcagctaacaattataagatctggacaaaatattgaaagaaaaactttaactgttttaaaagccatggaaagtaaccaaaaaagcaaagccagatgagagcctccaactgcaatgtgtaattgtatgtttgtggctttcttgccttatggtgcattttcgcctcctgtctttcagtggcataggaaagaagttacgggtagcctggcagttggaaatttactcaggggaatcatggaagcaataagtaagatccaaaatctgagtgtaaactctgtccaaatcatggctaatcacgaaactatacatgcataggggagaccccaggggacccagtgtaaaaacaggcagaaacaagagcggaatctgctcctgaaagaattgaaccctgtgagatttgtgattgcttctttttttgacagtacatttctcaacttgcatgcaatttgcgtggccaacagctggcttgaggtatcagagcacagaacatattgttgacagaagagttaaaatttagggaacaaacactgaaagcaagataacatcagaggatgtaggccacacatctcagtataatctctgcccatttccttcactgaccaccagactacccaggtgcagaggggatgccctgggcatcaggttagaagaaggaagcagctggaaggtgaaagaactaagcaaaatcattgttgcttactataagggagacacacagtaagtcctcacttaacattattgataggttcttggaatctgactttaactgaaacaacgtataacagaacatttttttctcatcagttgtataacaaagcagtgttaaaggaaacgacagtattcaaggaccttgctgtttgtgttttgcttcaagttgctgtttccaagacctatcgatggtattaagtgaggacttactgaagtttgcagtttgagtccatacgagcccattagaatcatacaacatgaaaaccaaatggtcctttgaaagattgcgacaaaatctggttgctataacttgctatttacaatgtgtattttcaactaaaaattattagatatgcaaataaagtggaaatatgactcattttcaggtaaagaaaatcattcaatagaaattaaccccaagtatagccagatattggatttagcaagcaaggactttaaaatagccctaaatataatcagaagaatttaaggagtcctatgttcaaaggattaaagaaaaatacagtcacccctctgtttccatgtttctgcatttgcagattcaaccaaccgtggatcaaaaatagttgggaaaaaacataataaaatttacagtacagcaataaaaaataatgccaataaaaaacaataaagtcttgttcagtgaatagataggagatcaaaacagagaaacagatactgtaaagaagaagcaaaagagaattttagggttgagatcagtaactggaatgaaatttttatattatattttattttattttattttgtggtggagtcttgctccgtcgcccaggctggagtgcagtggtgcgatctcggctcaccacaacctccgcctcctgagtagctgggattacagacatgcaccaacatgtctggctaatttttgtatttttagtagagacagggtctcaccatgttggccaggctggtcttgaactccctcccgacctcaggtgatctgcctaccttggcctcccaaagtactttgattacaggcgtgagccactgtgctcggccaagcaaaacagttgtttggagatggcaaaagaatcagttaaagatagattaataaaagtcatctatttgaaagagaaagaatggagaaaaataagcagagtttgagagacctttggggtaatacaatgtttgaacatgcatctaaatggagtctcagatggggaataaataatggctgaaaacccccacaactttggtggaaagtatcaatttacatatccaggaagctcagcaaacttcaagcaggataaacacacacaaaaaaacagatgttagcacataagtggtcaaattgctcaataccagagaatttcttgaaagtagccagaaatatcttaccagaaacaatggcgacctctagccatggatatggtatattcagctgaaaggcaaaaagctatcaaataagaattctgtgttcagctagaagtgaaggtaaaataaatacatgttcagataaaccaaaactgggaaattttcaccagccagccaccattgcaagcatttctaaaggaatcaatttcaagggaaatggcaccagatggcaagtaccatctacaggaggaaatcaacatcaaaactagtaaatacataatttactctaatttttataaactatatttttccttattttaaaaacaagtacctgtgacttgaagcaaaaattatgatgctgtattgttgggtttataatggagtttgggaatttacatattgtacaatttttaatatgtaatgacatacaaattaacgtgttaatagtcttctaacttatgaacatggtatatctcattaaggttattgtttaattttagtaatgtataataggtttctaaaaaacttaaaaatgtggtaaaatatgtacaaaataaaatttgccattttagctgtacaatttggtggcacagttgcattcacagtgttgttcgttcattactactttttccagaacttttctatcattccaaacagaaactctgtacctactaagcagtaatttcccatttcccttccccgagtctctggcagtgtctaatctactttctgtctttatgaatttggctattctgtatttcatatatagtagtcccccttatccacagttttgctttccacaattttggttatctgtggtcatccatggtctgaaaataggtgattatagcacaataagatgttttgagagaccacattcatataactttattacagtataattgttcttttttattattgttgttgttaatcttttactttgcccaatttacagattaaactaataagtatatatgtataggaaaagacatagtatatatagagtttggttctatctgtggtttcaagcatccactggggggtctggaacatatgctctgtggataagtgggggactagtgtacttcctgccctaccccttggtttggagtcgctgctgtatgaatataagggcagtctgcctgtgatgtgactctccattgatcacctgggtgtatgaattaattgctgtctataaagagtcttggaatgactattgagtttgctctgtgtatgtatacatacaacagtatggctactgttgtagctgatttggctatgatactgtagtatcttatttgctccaaagagatggagtagtgatgaaatctggtttactaggtggaagctagtgtaaaaatgggtactctgttttattgaattagcttgattggaaaaagtgaagtgctgatggttttgaaaatatgatgataatgatgtcatccttctggtttaaatattttgtagcacttgtggtagattgaatgctggtgtcggtagtaaagtcatgctgcagttatagtctgaaccagctgtactgttttgggtagtaacttagacagtagagaacaccacttttctaggcagggctcctcacctctcctagggggccatttcactgcatcttggagtgaatatacagagaggaagtagctagatcctaatttctacaagttatatcagttggggaacagttgtggttgtcaacctgtgttagggctgtattggttttgctttcagttgtgctataaagtggaaaaaattgaattgtttatttatctcttattgtgaaactttcctgtggcccatagaagtggcatagcaagctgaacatagctgggtttgttcttcattgcaagatcctacccccacctccctttctttcctgcctggtttcttgtgtccatactgcaagctctttttcccagttagaagtaagttagttcactcttaatagagctgatttacaaaggaattttatgtgtgtcatactgtttgattctctgaaaggtagatatgggactgtggtaacaatccagcatgatgttaactgtgctctcatttttaaatgtgcaaatggatgtatgtgaaatggtggtttgtctgatacaatattggctgctaagacatgtctgacctagagcctagatgtgttggcttcttaataatgctggatgatgtatatctggatcattctttttaattgactgagatgatgcgtatcttctacctcttgagagttaggaatgtaaagggcaagtgaaaaatttgttagatgctcttaaaagatgaaaatagatgtgctccatcactagtatagtttctttttgaccactatgtcaacctgtttagaaagtatattttaggccgggcgcggtggctcacgcctgtaatcccagcactttggggaggctgaggcgggcggatcacgaggtcgggagttcaagaccagcctggccaacatggtgaaaccccatctctactaaaaatacaaaaattagctgcgcgtggtggtgcgtgcctgtaatcccagctacttgggaggctgaggcaggagaatctcttgaacccgggaggcagaggttgcagtgagctgagactgcaccagtgcactggcatggcaacagagactgccaacagagcgagactccatctcaaaaacaaaacaaaacaaaacaaaaaacaaagaaagtatattgtaaacatcaaattaaaagaatttcctgaagtaactttttttttttttagtaggggtggggaagatatgagtagaagagaaatgaattggaaactgagtaccactaccctttttcttaccttctatccagaaatctttcttaaattccacagtatcagtgtaacatcttttatcttttgagctgtctttgagattctttactttgtctccagactgtttgttaggattctactagagaagttgttttcagattttaatttctctgctccattttaatcttctaattttttaatgaaaaaaaatccagttttattgagttcttttttttttttttttttttttgagacagagtcttgctctgtcacccaggctggagtgcagtggcacgatctcagctcactgcaagctccgcctcctgggttcacgccattctcctgcctcagcctcccaagtagctgggactacaggcgcctgccaccacgcccagctaattttttttgtatttttagcagagatggggtttcaccgtgttagccaggatggtctcgctctcctgacctcgtgatccgcctgcctcggcctcccagagtgctgggattacaggcgtgagccactgcacccggcctattgagttctaattaatgcataatttgttgagttttgacgtatatgtgcactcatgaaaccatcaccacaatgtatatatccagtgaatatatacatcacccttaaaagcctctttgtaatctcttcactccccacttcgcaggcaaccactgatttgctttctgttactgtaggtcagtttgtatattcttgaatgttatgtaggtagaatcatataatgtgtctcctcttttgtctgacttctttcaaccctagctcaatatatttgagatttgtctatattgttggatgaatgatcaaaactttgtttttgtttttttaactggggaatagtatttcatttttagtatatagtacaatttattttttgcccatttccatttagctgttattgctggattgggttattaacagttattgactattatgaataaagtggtcacaaatgttggtgcatggttttttatgtggacttgtgctcttatttctgctatgagtggaatggctgattcatatagtaggtgtatgtttacctgtataagacactgccaaacttttccggtttgtatcagtttatattaccacttgcagtgtatgagcattccagttgcttcacatcctcagaattcttggtatggacagtattttaatttgatttgttttagtagctgtataataatgtcttattgtggcattcattaatcattaggaaaatgcgaattatgttgaacatctttacctatgcttgtttgccattcacatctttttctgttgaagtattttctcgaatctttgcttattgatttattgatttatttattttgagtaagagtcttgctctgtctcctaggctggagtgcagtggtgtgatcatagctcactgcagccttgaactcctgagttcaagtgatccccctgcctcagcctcctgagtagctgggactacagatgtgtactacaacacttggctgtttttttttttttttttttttttttttaaggatttttttaaagagatagggcctgactgttgcccaggctggtctcaaacttctggcctcaagccatccttccacttcagcctccagaagtgcagggattacaggcttgagccacagtgcctaaattttttcattgtattgtttgtcttattattgaattataagagttctttctatgtgctaaatactagtcctttggataattacttgcagatttttttcagtttgtggtttgcctttttttctttacccatgtcttctcaagttcaagtcttttattttgatgaatacacatttatagtttttcttttatatttcatgctttttgtgttgtattttggcaggccaaataatgacccctgaaagatgtccatatcctaatttccaaaagctgtatatatgttacctgatatggctccacaaactttgcatatgtgattaagttaaagattttgaaatgaggagattagccaggattgtccaggtgagcccagtgtaatcagagagtccttgtaagaaggaggcaaaaccaatcagggcagaagagatgaatgactgcagcagaggttggaatgatgtgctttgaagatgaagggaaggggccatgagctaaggccatgagataataaatttgtgttgctttacaccaagtttatagtaatttcttaccacagcccatatagattttgcctgtaagtggaatgccgctgtaacaaatacctaaaaaggtgaaagtggctttgaaattggggtattggcagaagctggtagaattttgaggaacataatggagaaaacctagattatcttgaatgtacagatggtagaaatacagatgttaaaggctctgctggtgatgccccagaaggaagtgaggagcacagtagagaaaatgtgtatcgtcttagagaatacctaaatcatcataaagagactgttggtggaaatgtgaacattaaaggtgctgctgcttgtgagtgctttgaaggaaatgaggaagatgttattggaaactgaagtgaaggagatccttgtttagattgtaacagaaagtttacctgaattccgtcctgtagttatgtgactaactctcctgtgttgcctgggtatagcagcaatcatttatacatacatttgcagttctgttctgtacttgttataaagtagttttaattaaagtgacagatttatgtttttataaattaaatttatatttaaaaaatcagtatttagaacaaatatcttagtggactatattcttgagatattattattaaagtctttttacagcttcctttcaaataaatggatatgtaatttaaacctttaatttctggatttcctgatgacttttctttaaaaagaataatttttaaaaacacattttaatttataactaaaatggttatttaaggaaacacatatattagttttatttagatatatgcttttttattattaatttggaaagtggaggtgcattgtttggtagaataattttaaatggtagctcttgttaatggctaatatttattagtggatacaacttacaaatatctgtacattcagtctttatccttatcttccatatctttttcttctgcattctaggtttttttgtttgttttttggaaggaggatatataaagtaattattactacatttttagctgttgccaagttgaaagtcagtgtttagttttttttaaaaagggctatttagtgtggaattcctaaatattatagttccctcttctgtcctttcagagctattgcaccctgttaaattttttttgcattcagttcaaccagtgtttgaaaattaccaatagacttaaagtacttctctgaagttagaagagctcctatgtctgatgctatattgctagtatttctattcaggttggaatttggaatagtcattatcaaaagttatttgtaggcaaaagaaaacaaaaagaaaaaagttatctgtaggaaatattggagactggggtacaaggtaagtgacacagtgaagaagcacagacaatttcagaatatgagacacctaacatggtttctgcaaggtattggcgtggaaaaaaaaaaaaaagggggggggggcttagagagttactctccagtaaaagagatataagaagcagtactatagttatatacaaagggtagttctttggattctgtttagatcaaattaactagaaaatacatttttaaaataagatggggaatttgaatattgactggtattacttaatatcgtggaaatatacttattttgaatgtgtatagtgactttgtgagtatgaaaggcagtggccaaattatttagtgatacataatgatgtatatggatgagggaagtaatgacgtgatgtctggaatttactttaaaataccttggaaaaaatgaataaaagatagataaagcaatattgctaatatttcagtacctgttaaatataggtgacagtatatatatagaggttcattatactcctttctccacgttttgaaaaatttcatttaaaaattcaaaaaaattctaaaaaatattttttcttttagaaactaacacatgctgaaaaaattcaaattttgttcactgaccacgtttttgctgccaccactctttcaacatttaaaaaatgttcttaaatctttaaaaattcgatttacattttcattaagaaaatgaggcatttgctggccatttcatccttcctcttgtattttttctccttacctgctgccaccgtttactagaaaaatatttctcttgttttttctgtaccttctccttgaaatataaaagttcagtgaaggcaggagagattttagcccgtttttgtatggctgcatcttccttatccatacctgcatagcatgtggtaggtgctgagtaaatatttgaatgactgaatgggtgactgaatagactgaatgaaccagagttgtagaagacatggggtgacgtggggtttggctaaggacagaaccaaggagaacccctttgaaaattcactctgtgggatttacaggggaaaatgtgggctactacataagaaattaagataagaaatcatctttcctgtttgactgtaaagccttaatatttcaagcatttaacatattctcagcataaataattaagtttttttcttcttttttctgaaacagtctcactctgttgcccaggctggagtgcaatggtgcgatcttggctcactgcaacctccgcctcccaggttcaagtgattctcctgcctcagcctcccgagtagctgggattacaggcatgtgccaccacgcctggctaattttttgtatttttagttgagacgggggtttcaccatgttggccaggctggtctcaaactcctgacctcaggaacccacctgcctcagcctcccagagtggtgggattacaggcgtgagccactgcgcctggccagtaattaagtttttatactgtaagttctctaattgtctttaatgtattcttgaaagtgttttcctcctaatttatttattactgcaaggcaatccgttcttgaggttctatttaaagtttatgtatttcttctcaggaaaaaaaattacttaaaactagtggtttgtacagtcaagatgagttttagaagctgttcttataatttctttttatgttaaacatatataatgtcacatttcccttttcctaacaactgatcttctttctttttcaacgtgtatttataagctagatttttaaattttgctccagggactgctgtagttgttctactacttaaaaaaagaaagatgttagtattgaacactattctgagacacagctgataacaattgtgctcaacaatgaagatggctaaaaattgggttttaaaaataacagtgaaaattcagaagcattttatacttgctattctaaagtgagtatttttctaatctctgctttaaaattactgaagtccttttaatgaccaacgctgtattttaaggaaaaaatgtgagcaaagatttttagtgattctaaatttgttttgctctctggatctctttaaacttttaaaaattattgaagaccccaaaaagctttgacttatgtgggttatctctcttgatatttaccctgttagaaaataaagctgagaagtttaaatagttttcatttattaaatttatttaaaaataacagctaattacatgtttacacaattaacaaagtctggcttacaggaagacagttggatgctcatatctgcttttgcattcagtttgttaggatattacatgctctatactctgacaaaaactttactttgtgtatactcaagagacagagtaaaaaacataagtaacatcttagtattatagaaataattttgatcttagaaactccatgtaagtgtctcaggaactcccagggttcatattttgaaaattgctggagtcatctctcagaggctatgtgtgacaaatggtgtttttaaatgaaacaataatttttaaaatatagtgtcaattggctaagtttttatttgatactttttttttcttttttttttttgagacagggtcttgctctgtcgtccaagctggagtgcggtcacagctcactgcagcctccacctcctgggctcaagtgatcttcccactttagcttcccaagtaggaagtaggtgggactacaggcaagtgccaccatgcctcagtaatttttgtatgttttgtggagacagggtttaccatgttgcccaggctgctctcatactcctgggctcaagcagtctacttgcacccaactcagcatcccagagttctgggagccggattcttatttgatacatttttacctttaggggaactataaaaattagcttatgcttatttatgcaaataaatttgagcattgaggaaggatgtctcttgttcagggttgtgtttgtagatttcacatcttttagactaccatttaagagaagtgatcaccttgtatcttaaggaaatgaaaatcatttgatagaaatggattgttaaatatttttaaaacaagttttgacagttacttgcaattaaaggggaaagcaactttaaaatgcattagttaattgaagaaatactcaagatccttaattgttgttagtgagcaagtattaatgttctttcctctttgaaaacgtatctgtacctagacaaagttaattataaactttttcataaacaatattatgcttctttttcttctaatttttaaaccttatctttgttggtttgccttccagctccatctatctgcaacctaagaatcaacaatactatacatcaaattagctgatgtggaaggaaaatgataaataatgaaaatacgtcatattgcttatactctaagttataattaatataagatccaagggatattggtgtgggggacgaggaggaaggaacattgcaagtcaagctatttcacaagtattctatacctttgaatcttttatttcatagttttctttagaaacaatagtgatgcagactgaatagctagtaagataactttcattaatagcagtatggcagggatttttaattgatgcttgttggtaatattagacacatgatttatttataggcttttaattagaaaactaatacaaatatcaaattgttatggacaggttatacattttttggcaacacaaatcctaccaggttttaatgcctggtacttgtttttacattagtatgtggtattaattttttcattactactcttagcttccctagcttacccatttgctaggagtacatagcagcactaggacatttttcgctaacgtggctggcatgcatctgaaagtgtgacagagcaggggtatagctagattttagaagttcttttgctcatttaagaaacttaaacttcagatttgatagtacttcagattctacataatgtgctctttcgatgaatagctgtgtagcagcagcacaggaaaagcagagttcttggcaatacccagttcaattgcatctgcagatgaggatttaaaattctgtgcagcagacagtacctccagcaggcgagtaaaaggttactgcattatttttcttgtttgttaccatagcctttatctaccaaaggatttttaaaaaattgaatgtgaagtgcatgcctgtttttagatattctggaaattgatttaagtataacatgaatatcaaaagggcagtgcaaggtgaaggcagaggcagtgctggatctttttagctttaaaccagggaagaaattgagcatgttacatacacaaagaagaaacctttcagtgtcacagacaatgggaagttttaaaatacgtattttaattcaaggtgtttattatttagttttagactatcttccccaaaaagggtgctcttacattagcaatcattggtctatgaatgttcatttaagttacagtaatttgaagtatgattatttgtgaagactataaagtatttgtattaatgcgaccctcccttgtcctggaaattgtaatgtggattaattacattgtataaacaaatacctttgtgcattattggcaaaggtagaatgcttaagtataaaaggtatagtatagctagcatacttaatattgttgcttatgttttcagggtggtttgtggtttttaaaataatcttttaaataaaatgcagacaagtaagtgggagaaaattcagtatcaaaacaatgaggtttttgatggctttttacttgtggtcagtacctacctatatagccatatgtccttttttctcattaaaactcttctaacatttatgatagcaggagcctccttggattatccagcttgtttattacatcattatctgcaccagtatttttgctatagatttttattgttcattacttctcaagtctacataaagtatccagagggaatttttgcttgtatattttattcagttaccctcctaccctcttccatcagtcctcaaagatttgtattagctactgtctacattttgtttatttaaaaaatacttaggcctatatttttgtattgtagattgaataataagattgaaaacaattctgtaaattaatctatagtatagaagtaaaatttgttgattattcttactgtgagtatttggcaggcttcttgaattgtagagtcataaaaggtcagcagttctgattggtaagttaaagaatccatccagtaatttgctccctgttgtttctcaaagcaatgcagcagcagttcttttccagatattaaacggttcttgaacagtgtgtgtggagacggggaggggtggagctcattcctgttggttgttgaaatcgtgtgcatttacagagggaacaggttggataggctgagctgaaggagtaggcaaacagatctaaactgatggaagacagaaagtggttagtataattttggggtgtcttgagtttgctcctttgttaagtacttgggctttaatataaaacaatttaaaatattaaagcaccttaccttctttctaaagagtagctgggagacttctgttttaatatccagtttttcttggcgatgagtttgtggctggcatcttctgtagcaaagctgttgttttattcttttttagtagtgtttctttcagccagagagctgcagaatgaggcagtttgacaattttgagtatatatgtgacatcaccagttctgattttttaagtcaaattctcatctaatgagcaagtcaatgtgtggctgtggctgcagcattatttcttctttaatgaagaggttgagccgttcgacctgctgctccgatttgttctcttgtagccatacatcagaccagttgcagagattatccatcgaatgtcccaggcaaccaaaggtaactacatagatttatttcaaataaaaatatgcaatgaaaatggatgcattatgactaagaccaaatgattaaaaataaaagaccaattaaagatgttcttagcagttttcttgaccttgcagtagatatccaatatcattttgtcatcatcagattgtgtagcaatggaaatgcactgcagtaattgattttgtaataggatcaggtgatttactagctgcactgacaaccacttgcttgcttgctctgagctgtggagcactctaatggatgttgtgatttcagcctggagtttatgtgagactgccggccacttaaagcagcagcacttattttaaagattagattagtttttctttcttgttttcttcgtttcaagttttgtgagtagcctcagtaactttatggttaagttgtatgccttcatgaaattttagagattatatattatttcattcatcaattcatagtctttctgctccatattcctagctaaatatcaacatattctggtattgaccatgagagcatatctctaaaatatgagagttattggtaactatgctgtgttatctaaatgaagtggaatattcctcacattcgtagatttcattagcttcagactctagctgtaattagaatgatggtaaggttcttgatctcttgagttgtactgcagttgttttgcatccttttttggtcttttactttcttagtggtttcatgggtaaggcaccatttaggaaatatgagttgtattacttctaagggatactgatgaggatatataaagctattttaaagtagtgtttaaggatatagcaaattaaaaatctaatatcaagtattataaatttcaaagtgattattttaaaataatttttgttttccttttctatgccttttaaacaaataattggttcaaatatagaagtgtaggaatattgctaactgtaaaatagaactactgtcatagaaactcagatgctgtcaaagactttgattacttaaaagttttgctgatggtgttagattagaaaagaaactctcttccactcccttcctcaccaatacctctacctcatgtaaagtgttttatacagactccaccacataaaaatactgaattctttatcattccctgtttctgttcttgccatgatagagacaccatttctctcaaccacatctaaaaacatttacaaaaattaaataagattaacaatttactgtagtagaaacggggcataaaattgtcatcacatgtggtattcaaatcaccatgttaagagaactttcctttttgatgtactcaaatagtcacttgtagtgtttgaagccttagtgtttctagaaagttgaaaatattatctgtgctagtctgcacatttcctttaattcagatactttaaacattaattatggaaaattgaaaataatttaaacactagtatttgtaatcttttattattcaacaggtaaaaagtttatagactctcttgacttccaagaaaaaaacccttctgtgaacactgatgaacataagcatgttaatatcatttaggattcggtcaaggatgtgtctgaatttcatatatattgaaaatgtttaatgatgggccaccagcaaattaatcatggatatgttttactggagtgccgtctatgacagcttcttttcatgatgggttcagcaaataggaaacgaggaagtaaacaccagagtgttgactactttttataaaagttagaaagataactatgattggtctgtgattagacagtattttatgtaaaataaatgggcagcgtgaagttctagcctcagtggagctgccttttctaaagagccctggatcgagcattaaaagagttggatttaatttggctctgccattaatctatttgataaccttgatcaaattatgtatgaacattttaaagtcccttaaacatgctcttaaaatgtccattaaaaagatttcaatttacccttaccagtgggaagtattagaatacttgaccttgaagctatagaagttagagttaggaagaaggatgaagtttcttaaagaatcaagttgtaggtgatgttaaaacctctcctttcactttttatgtctttttttgggggggtgggtgggtaacatgtttttgctaagaatactgttttatctctttgatatccaatatttcctaagtaggatagtaattctggaaattatcctagtggttaatagaatagacctgggattaaaatctggtgctgccaattttttctagactttctaacaaagataatgtcattagggagtttattcagtacctaggatattttttagtaaacacgttttaagaaggttggccattatgttattggtgctttcttcctgtatggcctattagataagcgctgtgcagtcatctttgttgcctaggcaaaatggtttgtaggttcattgattgaactcttactttggaccaagtgctgtactaagcactttggttttttttttttttttttttttttttttttttaaagacagagtcttgctgtgtcacccaggttggagtacagtggcgcgatcttggctcactgcaacctctgcctcctaggttcaagtgattatcctgcctcagcctcccaagtagctgggattacaggcacctgccaccacacccagctaatttttttgtatgtttaatagagatggggttctgccatgttgcccaggctgatctctaactcctggcctcaagtgatctgccccgcttggcctcccaaggtgttgggattacaggtgtgagccactgtgcctggcctgtactaagcacttctatgttaattatgtcatttatgagaaagactctaatgattatttttaaagatgagagaactgaagctcagagaggttctacaactcctaaaaatcacacaactgggaaatagcagaaccaatgtcaaaactttaggcatcagcctgctgctcaaaggaagtgctcattgaagcgcttcggattttgaaatttctgataagtataatgcaaatattttttaaaaatcccaaatctgaaacatttctggttccaagcatttcagataagagatactcagtttgtatttgtactttcaattaagctgtgagtgtagagatgaagccaaatttcatctgaagatgtaatataaatgattacctaatttttttagtcttgaataagaaatgatttgttcacctcttactcagcaataattcgtaggaaaaattattcatataaagcatatttgtactatttggagtaatgttttccaaagtgtggttcagggctgggcacggtggctcacattggtaatcctagcgctttgggaggtagaggcagatgggtcgcttgagtcgagaagtttgagaccagcctgggacacgtggcaaaacctcatctctacaaaaaatacaagaaaattagccaggtgtggtggcatgtgcctgtagtcacagccatgtaggaggatcatctgagtctggcaggtcgagactgcagtgagctgagctattattgtgccactgcagtccagcctggacagcagagtgagaccctgtctcaaacaaagaaagtatggttcaggaatcacttgtatctatcactttggatggtggttaaaagtaccagtttacatcagggcacattgaaagaatcaccggaatgggatccagagatctttgtttttaaataagcctctcacatgattcttggttataccaaattgttacaaccaatgatattgagaatgttttcctgttttaccttgtttctatgccttgcagcttttcctttaagaatttgtttacaaaccatagatggtttgtaaaattatccttctgacttatggtgaaatgtaaattggttcttttgtggtactcattttaggaacttgtttaggattaaaagtattcataaactttgactcagaaaacctacttctaggaattatagagataattcctaaggagatactcagattcgcaggaagacttaggcacaaggatgttcgtaccattatcatctataatggtgaaaaactggaaacaactcaatttcaaacatcagataaatggtttgttttttatatccatttgtttggactaaccatccagccataaaatgttatatggggaagattttgtaagacataggaaagtgctcatgatttaatgtaaagtctcgaagaatataaaattatggtcatacatatatgtacatatagtgcatatggtcttaggttaaatcatatgaaattaccgtttttgtaggtcaagagtagtagaatatcagcattttcatgtagtgtaacctaacatggcacccatttttaaagcacatgtttgtagagaagcacatgtttgtagagaagcacatgtttgtagagaacgctttgtagagaaggagctggaaggaaatacagcaaatttataaagtgattatctctggatgatgatattatggataatttgaaatatttcatctttacatgtcttatgttttcaaattttctactataccttagactcaacctttgaaataggacaaagtaaattaataaaacagggaacagttgcaaactttctctgaaaaacattaaaattttttagaagattgtaaatcctttaagctagtataaggacccctttatactaatattcttaactcttgtattaattattgagtgtgtttatgcaaatggatcatatatgcctttacctacccacccagcaaattttgattggattttaatctgcagtagaaagtgtctttcaaaagaagagaaaaaaatcaattttcaaagtttttaaaaagaattgggttcttaaaagaccgccaactatatgcaggagtcagttgggtagccacgttaagcacaaagccctgttttaggctgtgtggttttgatttttaattgtaatttaactgatcgataaaactctgtagattaaaattagtaatttgtgtatttattttaaaatattactgtatttttttgaaaggaaaatgtttctcccactttcatacactttaatcgtttaattttaaagaaatagcttaattaaagtaacagctaccatattctggttatctgccatacaccaggttttgggtgctttaatatgttttttgttttgttttgtttttttgagaaggagtttcactcttgttgcccaggctggagtgcaatggtgcgatttcggctcaccacaacctccgcctcccgggttcaagtgatactccggcctctgcctcccgagtagctgggattacaggcatgcaccaccacgcccggctaattttgtatttttagtagagacagggcttctccgtgttggtcaagctggtcttgaactcccaacctcaggttatccgcctgcctcggcctcccaaagtggtgggattacaggcatgagccaccgcgcctggccttgggtgctttaatatgttacttctttcaatcttcacctagaatgtattaattagatatgagctttgttttagcgttgaggaggcactgaaactattataagctttcctgagatcatattaagtggtggatctaggtttttacctcagtaatgtctaacttaaagacccatgttcttatccctgatactgtccttgttggtctgtctgaaacttttaagtgttcataacttgggaaacaaaactgttgtataagccttcctaattcagactgtcaaagtgtagagcaaagtatataatgagtcattattgctgatttttattcaactatttgttacctgtcctttaatctggtcagtacatgtgtactggcatttaacgatcgcctgctaatagctaacctcttgtgtaaaccactggaaatactaaaacagagaagccaaaaattacttccttggagtttacagtcctaatgagagagacagacaacaaaatagttacaaggtatttcagaacataggtatggaatagaacctgttggaacagaaagtttttaaactaataatgtttgggagtagtggtgaagagttacttgggaagccatcttggaagaggtgaatttagaaggagccatagatttcgtttttgctaccagcctaggaatttacattttaatacaagaacccctgcctccagagtgatctgtaattctactgaagtttgagaaccaccactgtagattgtagttgatgctacgagaaagcatataataccctgaggaagctgtgtggagtgggaaatgagaagttggaaacctcagagcaacaacattgctttgataagtaggggaagttatatgatagttacgattaatatattataagcaatatcttttagtgcctagaagtaagattggaaatgtcttgaattttgtataagatcacaaaacttacaggaagaatatcaaaattccgattgaggttacttttttctgggagggatggtgaaatgttattctttaccctacaatgtgtgattaagagtgaataaaaatcgtgttaaccttttaaagataatgtttaagaaacaggtttaatttttaaaagaaaagtgagcataggagcaaagttttgtaacatattctgcttctatataggaaattaattcctaaattttggaaatgactagttgaaagtaatagcagtctatcctccaaagagagctttttaaaatttattttttagaatcagacctgggttaacttttagatataatttggcataaccagcaagtcattggttattcaaaaataaagattggaaataattgccatatagtcttggctctttgcgtattttgtgatactgattgttgcttcctcctttacttccattttattattagagcttcattaagagtgaattgaaaagaaaactgaattttattattttaaaaatctgaatagttggattggggcaactttcttcctatttatgagacttcactcatttatgttggtcttatattgacattaaagaatcagtgtcatatactggtgaatattaacaaagagacaagattaagattaaagaaattgaacctttaagcaggaggcgtgtgactaatggagcaaatttctgaaaggcttaggggtagagaaaatcaaagctactgatggtattaatagtcttagaaatgatggagattacatttttcctctcagagacaggcgacaaagaagggaggagtaaagaaacaaaacctttgagattgagatatcacaagttgataggtacacagctctctgaaagcttcagtcatctcaataaggagaggaacatacttagccatataagttcgagttgctgaatatacattctatgtaggtaggtctgtgtaattttgatacgtattttctgtttatcacagtggaaattgttcttgatcaatgtttttatttcaataagaaagatatattgccgcttccatcttaaaatttctacatagaagagacatgacgcttagtgaacgttattgaaaaaaaaaaaccgtataaacttaagcaatgaagaaagagaaacagtgtttcagaatgttacagtattgaacctgaattggtagagagaatagaaagtggtgtgaacagtcagggtccaaatgacattctatataaagcatataggacatgatttggagcagggagtgggatggggaatgcttagattacatctctttactagatgggtgtacttgttctgtgcaaggtaccattttctccccttttttgttagctcatatatcccttcccagttttgaagagtgctccactggtatttataactttagtgatttgacatttcaggatatacctgtttcagaaaatcataagaagttttaaaaatgccttgatgtaaaacaaaatttcaggtaaacttaaatattttaagtttttaataatttgtgttaccaaaattaaattttggttagagtttgacattcctaagtgatagaatcatcttaaacacatgttgtcaacagaaatttgaatgctgaagtcaatttttgttattgttattgttctgcttagtgaagaaatgtatcctctctaaccagggacagtatgtactgctttctccttaatactttgagattttcataaagaaaataatttcaaaggtgttgtgctgcaagctaaagagattatttctaaaggaagaatttgtcattctaatggttgtcatcttccttgtggcattattataaaattaagaaactgaaaaaaaatttttttagaagacatagtatataccagaatttaaatttagtttggcataagaggccacatattctacctatgctattacagtctgtatttgttattgcctaattttgactcattttgaatatctcactccagtcctttttattagtatttcttaaaacaaaaatgggcaatatgttggattgactgaaaataatttttaagtgtttagtagcaataaaatttaatcttttagtgcagattggtttaccccataacagcacatatggaagaagtaaagccaggtctgtatgaagcaggaggtgacattggactttttttttttttttttttttgtgacggagtcttgctctgtcacccaggctggagtgcagtggcacgatcttggctcactgcaagctccacctcctgggttcacaccattctcctgcctcagcctcctgagtagctgggactacaggtgcctgccaccacacctggctaatttttttgtattttttagtagagacggggtttcactgggttagccaggatgtctcaatctcctgacctcgtgatcggcccgcctcagcctcccgaagtgctgggattacaggcctgagccaccgtggacttctttaaataaccatagtacaggcagttacttttgttatataacagtatagttgtctctgaagaaaacctgaagctctgcaaaatcgggccctgaaaatcggtttctgagtggcttgttcctgtgtaaacttgtaatctctgtagtaacgaaaccagtacccattctaataaaaatattatatagctaaatatttaccccagttagttctaagttctaggatttatgctttctcctttgaaatgtaggtcctcgagaacattcatttctacttatgatgaaattgtgaaaactagtaaatctgatccagggtgtagtttttatcagttgcctctcaccccttaaaaaaaaaaattaacgtgggaaaaatattgtcatggattttctatctgtactcacagcttgaccagatggttttaacattgtggaatgcctagcagtttaaagccattaactagcctctggttaccctcttctggatttctagtttttttcttaaggtcatcatgtattgcttaagctctgtctttgtgagaaagcttgtccctgctggctttaaaactttactatgctggggaaaattttggactaaagtgacattcacattatactgacctcatcgcccaaatgatgaatgaatgaactaatgtacactacaagaacatgtcttaataggaaagtagactgttgtctgtagttgtcccattgttttatcattgctgctactatattgacatgaagtagcagtgggtgtgcatgtttgtatattttttctgtgctgttaaataagtccacgcacatctcccatgtgggtagggtagtactgggtgatggagtaggtttaccagtaccatttgtcccttactttacttgtatagctttatgttttaaaatagagtgtttttgtttttttgagacagagtctcagtctgtcacccaggctcgagtgcagtgatgtaatctcggctcactgcaacttttgcctctggggttcaagtgattctcgttcctcagcctcctgagtagctgagattacaggtgtgtgccactcctggctaatttttatgtttttagtagagacagtgtttcaccatgttggccaagttggtctcgaactcctgacctcaagtgatccacctggcttggcctcccaaagtgctgagattacaggcatgagccaccgtgcctggccactttttgttagaattaatactttgaagataccatggatttggttctagaccactacaattaagcaaatatcataataaagttagtcacaagaatttattgtttccccagtgcatataaagttatatttacatggcattgtagcctattaagtatgcaatagcattctgtctaaaaagtgtgcataccttaactaaaaaatactttactatttatttatttattgagacagtcttgctctgttgcccaggctggagtgcagtggtatggtattggctcgctgtaacctccgcctctcaggttcaagcgattctcttgcctctgccttccaagtagctgggattacaggcacccgccaccacacccggctaattttatatttttagtagagaccgggtttcaccatgttggccaggctggtcttgaactcctgacctcaagtgatctgcccacctctgcctcccaaagtgctgagattacaagcgtgagccatcgcgcctggctaaaaaatacttgattgctaaaaaatgctaacaatcacctgagccttcagggagtcacgatcattttactgctgtagtcttgcctcagtgttgatggctgctgactgatagagtgatgttgctgagggttggaatggctgtgtcagtttcttaaaataacaatgaagtttgctgcattgattgactcttcctgtcatgaacgatttctctgtagcgtgtgatgctgtttgatagcattttatgcagagtagaacttctttcaaaactggaggcaatcctttcaaatcctgccgctggtttatcaattaagtgtatgcagtattctaaattgtttaactttgtctcaggaaactactttccttgctcattcagaagaaacaacaccttatccattcaagttttattatgagattgcaacagtttagtcacattgtcaaactccagttctaattctagttctcttgctatttctacgcgcctgcagtgacttccgccactgaattttttttttttttgagatggagtctggctctgtctcccaggctggagtgcagtggcacgatctcagtcagctcactgcaacttgcgcctgccgggttcaagtgattcttctgcctcagcctcctgagtagccctccactgaagtcttgaacccctcaaagtcatccacgagggttggaatcaacttcttccaaactcctgttgatgttgatattttgacctccttctgtgaaacacagatattcttaatggtatctggagtgcagttatccttctcaaaaggttttcaatttactttgcccagatccatcagaggattcactatgtatatatataaaacagctaatgtatttctaaaacaagactggaaagtagaaatgacccttgattaatgggctgcagaatgggtattgtattattaggcatgaaaacaacatgaatctccatgtacatctccatcagagctcttgggtgaccaggttcgttgtcattgagcaatgagtaatatttgaaaggaatctttcattctgagcagtaggtctcaacagtgggcttaaaatgtctagtaaagtcttgcaataaatggatgtgctgccatctaggctctgttattccatttgtagagcacagggagagtatatttagcctaattcttaagagctctacagtttttggaatggtaaatgagcactgttctacctaaagtcaacagcagcatcagcccctaacaagagagtcagcctgtcctttagtttggaaggcaggtattgacttcacctgtccagctagaagagtcatggatggcatcttccagcagggggctgtttcatctacagtgaaaatctgttgtttagtgtagccaccttcgtcagtgatcttagttagctagaccttttaggtaacttgctgcagcttctgcatcagcatttgctgctttaccttgtacttatatgttatcaagacagcttctttttttaagcccgtgaaccaacctctgctagcttccagcttttcttctgaagctttctcacctctctcagccttcatagaattgaagtgagttagggccttgctctggataaagctttggtctaaggaaatgttatggttgatttgaccttttattcagatcactcaaaactttttccatatcaacaataaggctatttcactttcttatcatttatgtgttcactggagtagcacttttaatttccttcaagaacttttcttttgcatttctagcttttgatttaaagtgagactcttcctttttgcctgaatgcttagaggccattttaggattattaattggcctaattttaatattgttttgtcttaggggatagggaggcctaaggagagggacaaagatgggggaatggctggttggtgtagtagtcagaatacacaacatttttgccgtcttccatgggtgcggtttgtggcaccccaaaacaattacagtagtaacatgaaagatcactgatcattgatcatcataatagatacaataataatgaaaaagtttgaaatactgcaagaattaccaaaatgtgatacagagacatgaagttaccacatgcttttgaaaaaattgtgctgatagacttgctcaacataggattgccacaaacttccaatttttaaaaaaatatactatttgtgaagtgcagtaaactgcagtccagtaaaatgaggtatgcctgtaaaacaaacccgcagaaaaaaaatcgtctactcagaagagcactgacaagtatcattgaattattatatctacaacttgctaatcaagctaatgtagtgtgagttgtagatacaaccattttaatgcaggggttcctgagatctgaaaattattttaagtgtgcctccagtgtagaaagattgagaaaggttgaaagtgaagatcaataatgggatgaactggaaatatgcctacctgataatacaaagcaatgagagaaacatagtatcacttccgtcatattcctgcctgcgatgcataatgtgaatccaaaattagacaaaccaaaattaagggccgttatacaaaatgaatggcttataatctttcaaagttgttaaaggaatgaaaataaagaaaggatcaaggaagtatttcagatacaaaactcaaatgggttctgaggaatagacagtctgatatgtcagtgccattttccttactttaacatttatattgtgattatgaagaagtatgtcctttttataagaaatgcatacttacgtgtttggtaggggatggtgcatcatgttggaaacccctcccctcccctcccctcccctcccctcccctccccaggcctctcgttagtagtttccaaaccttttgcatgctgaacccattgctcaaatgaaggcagaatattaaacatgtaaaggaagagcttgtgtgtgggggggggggagcggggggaaaggaggttgggggaacagagaattaggaacccctgagtgactacagaacttctgaaaaccactgttctgtaggataaaatctaaactctgaagcacattatacaaacctctatctatacacatcagtttgtctgaaattgtatatttctcaacaatattgatctatttgtatttcaatatgttgtatgctctcatatgtctgtatctttgcaaatgatattttcttctctttaacgatttttgtcttaccctgctggtaaggaagtcaccttccctctcccttctgtatatccttgtcaaggaactatggaaattgaatggcataggtcttgatacaagaacctctcttttctctttataaaaaagtcagtaaatagtggtggaacattcaggagaaagttggggacatagagactaagaaggcaatgcagtattattaggacagggggtaaactaaggcatagaggacagatttgatgaacatattatcttttttatatacattataagaagtagaaggctagaaaagtgaacatgcttgctattgcacgtctcactttccaatttcatgaagaaagcaaaaactacttctactttattacttctttttgatgttaattggtaccacatagcaagtttgcattatagtactgagctttaaaatgcaaggatatactcatgtatatacacagacaataaataataggttcttttccacattcaggaagtgaaactcagctgtccgtacgacacttactgtccatatataggcatccctcagtatctgcaggggattggttccaggaccctcacaccaaaatctaggtatactagtcctgcagaaccaacatatatgcaaagttggtcctctgtatacttgggtttctatatcccattaatgtggagaatacttttgatatgtggatatggagagctgactgtatttattgaaaaaaaatccacgtataagtgcacctgtgctgttcaaactcatgttgtttgagggacaactgtattctgttacttcctatagtttctaaaagcattaaccaacaaatacatgtaagatgcgtggaataatttttccatttcctagaaaatagtcaatcatttttttttcagattgtgaactagaaagcatagtgaatatagatttttaaacatgaaaatgtaagtgtccattttaaattgttatggatatttctgtttgctgaagttcaagactgaagactactggaaaaaatcctgtgactgagagcacatagattccttctttttaggtagagcgcaacagtcaccatgacttcagtgtttgcatatggaagctaaaatctgtctgagaaagggcaaacaatcacaggagtcattgatttatttatttgttcatttttaatgcaaattttaattgagtgaaaaagtggtggaaaaaacagttgactaaagaagatatgagaagggttaatacatgaagaagtcctcaataatattagccaccatggagatgcagattaaaaccacaatgccatactactttccatccacaagaatagctaaaattaaaagaagtcagtcaccactgaatgttggtgaaaataggaagcagctgaaactctcttacatggctggtggaagtgtaaaatggtaccattcctgtggaaaactaattgacagtttcttagaaaaaacactcaccctatgagcaagcactcctagttatttacccaggagaaatgtccacaaataggctggcacaagaatgcttatagcagctttattcagaatgaccaaaaactgaaaacaactccccaacattcatcaagagaagtgataaataaattgaggtatattcatacaatagaaagtacacagggacagaaaagaatggatttctgatacatgcgacaacacgaatatcaaaaacagtatgctgaattaaggaaaaagacaacaaaagtaatgcagcatgagtcatataaacatcatataaaaatgatgtttcaaaatccacaaaactaatctgcactgatggaacagtagttatctaaatgtgctaaggagtgattagaatgggaatgggacatgagaaaagagggaactttatggagtgatgaaaatattcaatacctttttctttttgagacagtgtctcgctctgtttcccaggctggagtgcagtgtcgcgatctcgtctcactgcaacctgtgcctcccaggttcaagtgattctcctgcctcagcctctttagtagctgggactgcaggcttgtgccgccatacccggctaattttttttttgtgtttttagtagagatgggggttcactatgttggtcaggctggtcttgaactcctgacctcaaatgatccacctgccttggccccccaaagtgctgggattgcaggcatgagccaccgcacctggctgaaatattctgtgtcttgattaaggtgttgtatatatagatatatatgtttttcaaaactcactgcgctttctactttatataaaaagtacctcaattaaattacatattaattgaaggcaaattaaaaacatctctgtctgtaactctcctactgtgagtctgttccatgttgaagaatgctcttgtcttcttttctttgctcttcttggttgggacttccctgttctaatatcaggtatgctcacgttagaaagctaggacagaggcctagaatactgatttttttccatttcaacaggcaaggtgacagttttcctagagttcacaactcttggtattccaagtcagcacatagttcacggtgaaacattctctaggaggtagagagaattgtgaccgaagaatatgataggaccaaataagtagctatactagaaggaagcacatttgagaccactaccctaaacagccaatgactacagaaccagagaaaaccagaatctgccaaatttcagaaataactcagcatctttacagggagcctctacacaatcacaatgagactctgaagagtagcaagtactgattcaaatccttttagctgtggactccactctgcctcacaatcattttagaagagggttttgagctttagaataagcccaccactgtataaaactagtgagtctgttttttaaggtaggattcccatcttagtaatcataattttggcatggcaattattaaatatgaaccacaaggagaaaaaaatgtcctcttgttattactttttaaattaggcattttaatttcatttactactgcttgatactctcttctctgggttcctgggaggtttcatttggtaagtattgttcagtaaaaattatgggacaccattgttttggactgtgctcctgccctaggccccagtggaccagatcaaactaaaatgtagtcattcatgttaactgccacataatcaaatagaaacttccaggaagcagacagatcccaaagcagaccatttttttccctgagaacaggagattccagtctagctgaatcccctgtgctttaaacccttagaaaaagtaaactgaagtaacctgatattaacaaatcagcttttttttcctgttctgtttccttgttcccaccttacaaaacccactgttgtgctgttgctcagtaggacctctcattctaccttgcagaatgggggctgccctgattcatgaaatgcaaataaatgccagttagatctgtaactaaactgttgtaattttgtcttttgacagaatgcagttaaaactcaggtatatccacactgcttttatttcgtctccttcccatttcagaaactcctgtctagatttcttttcttgctctctaaggcaaacaattaccacagttagatgaatatgcctggcaatcgctattgacagatagactctcagaaacactcgttccattttccttttcaaattcttggcaggaagactgggtggatatattgaaggaggtactgtttgattgtactgcacacagtgtggtttcaaaaatgtgtatttgaattggaaaagagaaatatatgtcaggcactggggatacaatactgaccaaaggtatataccctgaatccaaaaataaatataatgtagttgctgtaaacatattaattttaaagattgtcagaagaatatccatcttagttagacctttgcaagatatatagataagaaaatttctgaaagttacataactaaaatagaaaccagtcaaaatgaacttcattctaccctggtttcttccctctaatgttcttatctgtcaatactgtgactgaaaaagccaaacctaccaccaccaccaccactgacaaaatccaaaaaactacagatatctacaacattaattctaacaccaagtaacattataacccacttaaaactgttcttttttctttctttttttagtacagacagaacttttttttttttaatggtaagaaatatcatgattttcttataaagaccttctgtatgtacaaataaagctatactatcatagtggataaatcagtaaacaatatcttatgtaatctgtaatttttcacacaagaaatttaaaaagtttttcaggttatagaacagtatttctggacacacctaacaaggcagtagctgacattctcaaacttcaaaacaccctcactttcaatacaaataatgctgtgttgtacatttaacctaaaactaagagaaagcattgttggttggccacaacttgaatttcttattacaatgaggactgcttcaggattttaccactaaactcagccgttcagagaactaggaagatagtaaccatggtcactgttcttcaaaagcactgatttcagatttgttgaagggatatttcttagatcttcaagtttctgaaaaggtttccttcattctggttttctggatttccttgtcattctgaagttattcctcttgcacaggcaaaattagagtactcattcaataacaacaacaacaacaacaacaaagccataaaatatgccttcttttttctacaacaccaatttagctactcaagatacagaactaaaatattagcagaaaatcttgtaggtatgaagccagttatgtattcacattagtaaagatgattccactgcattcatttaatcagcaaacatttacagaatgcctactacataccaacaatatattagtgactgcatattcatttaatcccgaactctgaaatcattattcctggtttacaaaataggaaacaggcttggattaaggttgcttgcccaaagatttatagttagtaagattcactaccaagtttgaaactcagatatgtctgagccaaaatttctgctccttccactaagccaagctgtcttccagcatggcttccagcatacttagtatcttgctgtatcaagagaaaacaaggtacaggtgacagtgacgtgatgccattatgactatctctttttatataaaaagacccttgttgagataactacaactctctctttctcagacctcctacacattgcttttccttcaatcatcagggtcatgagccttctcccctctcctcccccgccgccccccaccacccaacttccccctaacaaagtaaaagaaatcaaatgagatttgattaaagtgttttgtctagactgcttcaggactaaagggtcttggtaatttgtaccagcacctcacttcttccccacgcccaaaaactcacgtacacagtttgattgttcttgctttctaaagtgttctcttttttttgagatggagtttcgctcttgttgcccatgctggagtgcaatgacccgatctcggctcaacacaacctccgcctcccaggttcaagtgattctcctgcctcggcctcccaagtagctgggattataggcatgtgtcaccatgcccagctaatttttgtatttttagtagagacggagtttctccatgttggttaggctggtctcaaactccctacctcaggtgatccacccgccttggcctcccaaagtgctgggattacaggcatgagcccggccaaagtgttctgaaagctatgtgtgaatctgttaagtacaatctatacttacatttgattataaatattttgacgatctgaattggtaagattttaggcaaaacaacaaaatcaaatcccgtggactctgacccaaagaatatattcagctttgtttgaatatatttcatcttgcttccttaccaattctcaacacaactccagaataactgttattaactcttattgaatcttattttgtactaagccttgttaaatgttttacctatgaattgtgatttcattgttacagcaaacctatgatatagatgctatataacagatgaagaaatgaggttcagaacctgtgctacgctgcctcgcaggcacaatccatctgataaaagtagtcccttgaatctctctttctgaattccagtcccaggacagtggaaatacacagtattgtggtcaaagataaaccattcttggctccaggtgagctaatggttttagataagaataaagaatatttattagaaataaaaaattttgtaagtataaaattagactaggaaaaagctcccggttgaattttatatttataaaatcctttactgaagttgaaatcttctccttgaaatttctgcttggtatggcctctctgtttgcctcacaagtgaaatttaatcctaatgttaccttattttccaagcataaccacaccagtaggctgctttgtcatgagtttagtatggtgtcagtagaaacgagaccagcagtcattagggttacctaattcaaatcttgactcattttgggtacttcctacaagattagaataattatcttccaaaccaaaaacatacccaaggttccatgggaaagggtgtagcttcaactctttttaggtggcaagaaaatcattctctaacctctcaaaggtcaggaatagggtcgatggtccttaaaatttggtctggggaccactggttgctgaaccttttgcaggaggtctgaaaggtcagacctgttttcacaatacatgttaagaagttaatggcttttttgttgtcattcttttccaagtgtatttgattatacaacttgggatgatgttcctctgatagttaatgcaattatacttatgttttaaaagtttgttagttttgatttctaatacagtaagtctcaacagatagaactcacataacaaaacacttttgggagtccttcataatttttaagagtgtaaaggaggttctaagaccaaaaggtgtggaaactgctcttctaggtttcagaattatgaagcatacaaagactcttcatctggaagggtttagaggtaattttatctaatccccttattttacaggtttagaaactgattctagacatttaagttcccagactaatgtcacagaagctaatgaattgcagaggttaattggaagcctggtcttaacactcccaggttatcttaatgagttcatgaggatggcatatggataatgcacttcaaagggtgttgtaagtattaactaagttaatacaggtcaaatgcatatattagcactcaatgcacggccattgatcaataaatgctagtggttctgatcagtgagaatctaacctctgcttaaatacctttagtcatcagcagcttccactccctgagtaacatgttgcatttcttgatcaattatatttttacagaattcttcctttactgaagttgaaatcgtctccttgaaatttctacttggtatggcctctctgtttgctacacaaataaatttaatcctaattttatctagcttattttccaagcataaccacaccaatttcattaaatgattcctcatgtggcatgactttaaactccgtcaccatcctatttgtttttctcaaagagctccagttgactgctcctgtgaaattgtccatctattaatgtaaatgttttttctaattttacagagctccccgttgtattgtgtacagtgttaaaatagttttctgagatttcttgactctgttttcccaagtttcttgtggcccttctctttccttcgtctctattctgtgcggtttttatttcactcccacagtttctcattgctgtgaggccctgttatggaatgagagccctggttttgaaagttcacagaggctagacttctcttgtccctgtagtcctggctgagggcccactacacttgtctgttatccgagtgggcaaacgacctacccgttttcatctgctgggcggccggttatttggggggatccccctgttacaggtctgatctctgttgcttcctttgggaggccgaggcgggcggatcaccaggtcaggagtttgagaccagcctgaccaatatggtgacactcctgtctctactaaaaatgcaaaaactagctgggcatggtgacgtgcgcctgtagtcccagctactctggaggctgaggcagaagaatcgcttgaacccgggaggcggaggctacagtgagtcgagattgtgccactgcactccagcctgggcaacagagcgagactgtttaaaaaaaaaaaaaaaaagtttgtagccattatctctggaaatatttactcttcctcattcttctgcttatctctttctggaactccaactagatatatactagacctttgattctatttttccacacctcttaacctgtctagcttatttagtcatacatctctcaagctgcattcttccttggagctttatctgtaggaattctttgagccttgattgagtttatattgcttcagagaaaactaaggtttgcttcagctagttgcccaggaacattaccacctggttaccacttttaattaaggtcactgcttgaggatttcagagcgacacagagccctgtatgagggcctgtttatggttataaattcttggggtgtaggcaggggtggggatcctgcttctttacctggagctaaaactgagacagaaaaatccctgactgtccatttttgtgtggtgggtttattttctgttcaccctgaacaatgataggtgttcaccttgtagagctccccgctttatgtgggggtttcctattagagaactatcatatgtggcctgttggcttattttcttgccttcagcacctgtgccagtacataagttagaaacccaaggtttccaggtttcgaaaaactctcagggcaaaagttcgttttagcctatgcttagctcccagggtcctttgtacttggccatatggatttctttttttttttttcttcatgtcaacactgtggttatatttatctgtctaacctactcttttaatttttgttttatctggtattttaattgtttcatttgagaaagtttttcatagtatctgtttggtagttggccattctactaggagtttaggagttatacttaactccttccagggagatgttggaagatttttactaaaaatacaatatttaaggtgggtattgaagtactatcagaggggacctggggtggagtatgaaaacaaaacaaaaccttacaaacgggaataattaagagttaggatatgtgagatgtttggttttgctgggcttaaaatagaattgtggggtatggaattgctgcaaatgaggaagatggactcagagatgaggctcaagagaggccacatgcaacttttgagagtctttgtgtgtcacagtaaggaagatgaacttgtgaaatcaccaaagggtggaatatataaggaggaagctgacactgtcatatctatgccttaggatgtttattttggcagcagtgaaaacaaatcaggtagacaagagtggagcattgctcttcaaaatttagtcattgactggcagtaatggcatcatctgggagcttgttagaaatgcagaatctcgggtcctatttcagatttaccaactcagaatctgcattttaacaagactcctaagtgtttcatatgcatagtcagttgatgtctcaaccagtgacctgcagagccagtgatctgcaaagcaagtgaacgttagctttattttaaatttcaagaaagaagtcttaatttgcctagagtacatatcatttgatgttcacagtgtcactttatttgcctaataactagctgactctagatccttagcagcattagatttagtatttgaggttttgaccctatgtacctttgataagtagtgatttgtaacttatgaattaaatttgaattccttaacatgttgctagttacaaaactctagtgtctctagcagactattaagaaattgagcaggtttcgttctccattcactgttaacacatgcggtaactcaccaggcgcttaaaacaagtgggctgtggtgcagggtacttcttgtttagtgacagaaatgaaaagatctaagaatatgatggttttaaataaatatgataagagatgaagagagttttaagtaatagaactattctaagttttggatgtttggtttagtgtctctagctatatttctgtttaatgtcaaggcataatatattcgaaccattttatttattaaatttaataaacattgattgtcctattagtcaaagtttcagaaaaatgcaaaaagtcttgttctcaaaggaatgctccctggaccatgcacatcaaagtcgtgggtggctgcttgtgggggtagaaaatattaaaaatgcagaaactgggccctactgccgaatcccaatgtacattttaaaataagcagcttcctggccaggtacggtggctttcacctgtaatcccagtattttgggaggctgaggcaggaggatcacttgaactcaggagttcaagcctgggcaacatagtgggacccccatctctgcaaaaaatggaaaaaaagtaaaagcatctcccaaggaattctgccagcaggaaagttgtgttttcccccctcaaatctggaaaccataattgttcactttttgaccttttactggaatacagcatactctgagaaaagtaacaacactcaaataaaaaaatagaacattctattccagaacctcctctagtgactccttctagaaagcagtctagcaccccacccctccaaacacaaacataaccaccatagtgacttttaacaccatagattagtttttccttttttgaactctttaaagtggaataatacaatttatactctgtctgttttgtgttgtttttgcttagcattatgtctgtaaaatttgtctatattgttagctgcagtttgtcattctcattctcattgctgtgtggttctctgttgtttaaaagtaccagtggtaaaattatcatttatgtggtttatctctgatggacattagaggtatggtggtaccactgataggcaccaaaatcaagtttaataaccactgttgtatattatttgggaaaatgtgtactcttgacatgtttggcaaatttttatttctccaatctaaaatttagaaaattataattttaaaattatcaataaaaatttacaaagtttttactgtcaaatattatctaatagctcaactagctcatgctagagtaatacagccatgtaatacagcattaccaacttttctaagtggtctatagcactgaggaattaaatagcaaataagggaagcatttcttcatgaaatcatttcttttaataaattaaaaagaaatgagagtattagaataccaccattttgccaccttcagtgtattaatggatggaggcaatgaatatcaacagctgctaaaatcataagaaggaaaccagatgctagatgacacataatgaagtaacacattattagacaaaggaacaaaccaagttcaataaagcctctggatccagcttccaatttgccacaaataaagagaccagatgaatatgctgaattgcattatgtgtaaataatcagtcaaatctacactacgaaaagtcagatcaaacataaattttaagaaaaagaaaagaatggaaaataaacctgtagattaaaagagactgaatagaagtagcaactcttcttaaaaatggtcaagattaaataatagttatctagggaggcttacttagctgataaagtgataaatacaggatgtaattaagaataatttaaaagaagtgagttacttagaggaagggaaggggctatgactgaatcagcacttggaggagcttctgggctcgctggcaaattgctgtgtggtggtcacaagaatgtattctcttaatatgctaaggtataaagtttttgtatagttttctgtatctgtattttattttactctaaaggaagtagttttttaaaaaagattatagatttgctaatctaatataaactcccctagagcttcatcagtaatagtctagtgtcatctggattatcttcagcaatcattttcattctaaattgtcatagtgtcatctcaaagcagaaagagacagagagtttgacaaagtgtcttaaaatgcctgatgtctgtcttaatgattctaatagaaactcttcagtacaatactcagacttactgcgcttacaactacataatggcttataaatttgggaaactcaacagatttgggaatacaacacatatcttgaagccagttaatttactttttacatacctcttcactattccactaccagtgtgcaactggtaattcagttgaattcataaagaattcactgagtttttctgaatttcttctgatttcaagaggctagaagaactcagattataaagcatcaggttatcacttttactatataggcacaccatggtttattgcactttgcttctgattttttttttaaacaaattgaagggttggagcaatcctgtgttgagcaagtctcttggtgccattttccagcagctcactgtgtgtctctgtgtcccattttggtaattctggcaatatttcaatctttttcattattattatacctattatggttatctatgatcagcgatctcttttatgttactatcataattgtttggggatgccatgaaccacacccatagaagaataaaagatggcaaacttaataaatgttgtatgtattctgaccactccaccaaccagctgttcccatctctcttttctcaggcctttttattccctgagacacaacattgaaattttgccaattaataaccctacagtggcttctaagtgttcaagtgaaaagaagagttgcatgtctctcattttaaatcaaaagctagaaatgattagtgaagaaggcatgttgaaagcagagacagggcaaaagctaggccttttgtgccaaacatccaagttgtgagtacagaggaaaagttcctttctttttttcactttttcgtatgtttaaaaagtagagacagggtctcgctatgttgatcaggctgttcttgaactcctgacctcaagcagtcctcccacctcggcttcctcaagtgctgggattataagcatgagccactacacccagctgaaaagttcttgaaggaaattaaaagtgctactctggtgaacacatgaattataagcaaaacagccttattgctgatatggagaaagttttagtgtctggatagaagatcaaatcaaccataacattcccttaaacaaaagcctaatccagagcaaggcccgaacttacttcaattctatgaagggtaagaggggaggaagcttcagaagaaaagttggaagctagcagtttggttcctgaggtttaaggaaataagctgtctcgataacatgtaagtacaaggtaaagtagcaagtgctgatggagaagctgctgcagcatgttatctagaatgtctagctaagatcactgatgaaggtggctaccctaaaagattttcaatgtagatgaaacagcactctattggaagatgccatctaagacttttctagctggataggggaagtcagtacctggcttcaaaacatcaaaggacaggctgactctcttgttaggggctaatgctgctggtgacctcaagtggaagccagtgctcattggtcattccgaaaatcacagggtcctaagaattatgctaaatctactctgcccatgctctgtaaatggaacagagcctggatgacagcacagtttgctagatatgttaagcccactcttgagtcctactgctctcagtgaaagattcctttcaaaatattactgctcactgacaatgcacctggtaacccaagaactctgatggagatgtacaaggagattcctattttttcatgcttgataaacataataacatctattgtgtagcccgtggatcaaggagtcatttctactttcaagttttattatttaaaaaatatatttcttaaggctgtagctgccgcagtgattcctgtgatgaatctgggcaaagtaaaccaaaaaccttctgggaaggattcaccattctaagtaccattgagaacattcgtgatttatgggagggggtcaaaatatcagcattaataagagttgggaagaaattgattctacccccatgggcaactttgaagggctcaagactacagtggaggaagtcactgcagttgtggtagaaatagcaagagaagtagaagtagaacttgacacagcgtgacaattgctgaaatctcaagataaaatttgaatggctgaggtgttgctttttattgatgagcaaagaaagctgtttcttgagatggaacctactcctagtgaagaggctttgaaaattgttgaaatatcaacaaaggacttagaatattacataaaattagttgacaaattgataaagcagcggtggtttgggaggattgcctccaatcttgaaagaagatctacagttaggtaaaatgctgtcacatagcctagtgtctttcatgaaaggaagagtcaattgatgtggctaactttattgttgtcttattttaagaaattgccacagccatactgattagtcagcagccatcaatatcaagataagaccccccaccggcaaaaaaattggactcactgaagtctcagttgatcattagcatttttttaagcagtatagtccttttaaattaaggtatgtacattttttttacacataatgctattacacacttaatagtagtgtaaatataacttttattgcactgggaaaccaaaacattttttgtgacctgctgtattgcaatattacctttattgcagtggtctggaattgaacctgcagttttctgaggtgtaagtattggtgaaatcattaacctcctcctctgtgtgtgtggtttcttcaactcacaaaggatagcagccccagtgaaagtcgttacagaaggcagataaactccattttttttaagactttgccactctgtgtgtgtgtgtgtgtgtgtgtgtgtgtgtgtgtgtgtgtgtttaccttatcctagtgggccttaccacacatttctattatgtgagatccaaaagtgatgtggtatacacaagagtaattttcaccccattcttaaaactgaaaatgatgtttaaatcactttttcttgggggaaaattcaaccccgtggtaattaagcttcacttttcctgaaattaatctttttataaattttataaacttataagaaatgtaatacattttgcttaagtaactagcataagccaaaatggcacttcatttttcttaggctcagcagatttaatttaataaagattataaattgattttaattaatgatgtttacgaaatgaaaattaattgacagtgttgaatagtaaagctgctaaaaccataagccaaagcaatatgaataaaagtataatttagacacaattttaaaccagattttgaaaaactttactcggtgatatggtcaggctcaattgtatgcctgctagttagattacttcttgtataggctgatatatgcataaaattattatttttgaattttaaataaaattatatttatctctttaatgtgaatttacctctttaatgtaaaaattatatttgttttttgtccttgtttcttactgcacatatcaattccaggtcaagcatgagataagcaaatcatgttaatttcattttcaactgaaataagactatttctgagcttattcttgtttgctgaataaaaaaagattgttcatatgtgcgagaaattgctgcagcataatgttcactgtttttctgtgagtagcaggataatcttttcttcatagaaatctacatcttgtctgggaaagattagtaaaactcatctttaatgtatgataaaactgcagatctttttccttttttctcaagtcaaattttcacagtcagaacattcagaaacaggatcccttttccagttctacacttgtgttcaagtgaaggaaaatctgttcagttttttttctgcagctcctccaggtgatttccagtaagacttgagacttgctgatatccttcctcactctcaagcccaaacagcaatagaaacatttcaagattttcttttgtaacatgtttttcaaaagctcattttccttttacatccaagaatgttgtcctttgaaataaagaaaacccaaccattttcttcacaccttacagagactgaaaaataagtactacgtaggctagtttctacagccattaatgtttaagtattcaacagactgtagcctactgttttctttaaaggacccatttcagttcaaataccctattgagaagttttccactgctaagtggattttgtgtgtgtgtgtgtgtgtgtgtgtgtgtgtgtgtggtaagagctgttctttaaatgagtgaaatgacctttgtttaatgaagttaaccattttataacatctttcagatgttaaaacattttgccccaagagcttttgatatcagtactcttaacacagatgttttttgtgttagtttgggtcttctgagacacagatgtcaagacaagatttgatgcgcagggaatctcccaccccaccctactaccctcctattgtcctttggagcaatccccattctagtacttctgccctgcttggtagcttgctgggagaagcccagaggcagcaaggccaggggtggttgccatggattccaaggtgcgatagatggaggctgtcaagcagttctgttcctcatagcaggttaggttgaggggatatctgagtagggtaccactgtggccactgcaccctcctcttcagcctttgacatctttccctcatgtggacactctttactgtgttacgcacccacagggatgttgtcactgtgttctgcctttgacctacgggggcttctttactatatggattattcctcatgcttcttagagtcaagctttgccaccttgcccccttgttggtctgcctctgttcctccccgccgccatccctcactacagaaaggctcttttcattctgctttgactcccaaattttggactacctgcctgccacatacatacctaacttgcttggctccatctaatagctttttgactaaatttttgaggaaaagaaggaagaagagccatctgggtttcttacatctgcaactagctagaatatgattccattcactgaaatagagaaaataataggaggctctaagaacaaatttgggccttacgtatgtgtgcatgttgtggggaaatagaagtttggatgttaaaataatgaatttggtgtgactttgggacaccttcttgaaattaataggctcagtagaggagtctgactggagataatagtatggggagtcattagcttctgtgtgatagttggaaccctgactttcaacgaaacaacaaaggagggaggagctagcgatggagcacagtaacagtgctgcctgagggatgcactgaggaatgtaccaaggaaaattaagggacaggagagagttgtaaagagagctagtgaccaacagaatataaagatacaaaaatggcaaaagagtctttgagatttaacaaccagtttaaaatttagttattatggagtaagcccatatcacacaggtggtggcgtatcacacagggattaaatttagccaaaacaaaatatgtattataattcaaataacaaaaaatatatattgtcatgtggactgaggaatagtcttttgcatcctttccagtaaagatatatatgattgactagtgaagtctatttaaattattcagtgtacatccataaaactttatcaattactttctgtgtatatgatgacacttgccagtattgcccattttgtgctgattttgttagtgtacattactaaggagcttttcatcaaattttgcagtccctgaaagattttcatctttaaagttacaaatgtgatttctcagctgcagtatagaatatatggaaccagaaatactcaaaaaataattgttttcgaagtattaatctttttagtcacagttgtttttttacatcctgtgctgaaaataacaagaaatttgatacagagggaaaaatgatttattatgacttagtagacaatttacaaatgtgtgttaagatattaggatatatgtcctaccagtttcaaaagtactacaagtgacttgttcaatacagagtacaactttaaaaaatgtatttaaagtaattgacgttgtgtttgtgacctggctcgaagttcttttttggtgaagagcatgcttagtacatgattgtctttaaaaagatctgataattatttactcagtaaatgttttgagtgccttatgagtcaggcatttttgtgggtgctggagataatagtgatgaaaaacaaaagtcccttttccttatagaacttaaattctagtgtgggaaggcagatagtacagaaataaattgatggatggatagatggataggtaggtatttgctatatagcaggtactatttttgcttttatatagtgctgtggaaatgagtaaagtcaggacaaaggaaaagagaaggtgatgctggggggtattattttaagttggatggttagggaaagtatcattgaataggggacatttgaacagagtcctctatgaaggaagtaaaagaaataaaaagaggaatagctagcacaaagactctgaggttgcagcacactttacccctcaagaaagaacggtgacagaaagggtggaacaaagtgagagagtagtaaaatatgagttcacagtggtggggatgtatgtagatgatagaaggacccaacaggcaaccataaaagacattggcttttactctgagtgaaatgggaagctcttagtgggtgttgagcagtggagtaataagataaaacttatttcatcatgcatttcaggtacttactcctaatcatagtaattaataaattattaatttggggtatagaaagttcatatatgaagtggagggtgttggctcttttaagaattcagtgaaaatcctaaacttcttgcagaaagtgattccaagctttgagttattcttcctgatgatcagtacagttggtttactttcttgttatctttgtcctaaagcttcttaaatcactttgtagctcaagcctaatggattatacctgcccatgtaaattctgaaaatgttaatgttgcctagtgattcagaggctttagtaatttacttaaactacttcttgttttattggtataaactgtattcctcagtgtctactatgatttcaaagttagtatttgccttggaatttttctttgaagctggcaactctagttcaatataagacaggctctcagagctccacttatttaacaactgtatctatgcccacttttattcctctaagcattcctagaataagcatttcaaatggttttgaattatcaaaggtctttttgaaaaacaaggttattgagaaaatgtttaaaaagttcatgagaaagttgtctaaaatacacccccctcccccaaggtaggcctgaaatgccactaaatcaacaaaggatagtcactaaaagttaccctgtattttctaggacttggggaaagtcatggagtagctgtgctttctctaagtaacctattttaaatttttttgaagagcaaaattattatacctctttttcatccagaatggagctgactgtaggcatctttttaaagtacccataggtcaggacctctctggactttccactaccttgaaagacactgccagaagaatgttgttccccacacttaaaaaaggaaaagtaatgttcttttctccagccattattttatgcaaagtccaactttattttatctgagctatctgaactttcaatttatttgaaacatttattgattaatatctataatggagcttttaatttctctgatcatttatcctttgttaggttagcagaactgtaacaacacagtcactttcacagccacgaatgatacttttatagtcatttgtggacatatactgtgcagagtatcacaaagtttgagccacctgacaaacttatttccagttgagatctgcagggtgatgctctgccttcttgttgcagctctcataccatgaacaagtgtcattttcatggtccatgaggaaaaagcattttcctcattttggtgctttttgttagtgattgtgcagtttagaatggcccccaaacatagcctgaagttctgtttaatgttccgaagtgcaggaaggctgtaatttgccttaaagataaactaatgtgtgttaaagaaacttaattcaggcattagttataatgctgttgaccatgagttcgatgctaatgaatcaatgtatattaaagatatatttaggcagaaacacgcataaaaaaggttatgcattgatcagttggcaacagtgttgtgaccagaggcttgtgagaactgtatttcccctaggagcaatggctcagtatttgttaattcagtggttgcagcaattttatagaacacaattactgtgaatgggaatcaactgtatttgattaacggtggtgatttatagggactttagataccagaaatgtttcataattccctttctctgaaatcactgttttaatatgtttaaatgcatgcatgcagagttgtgatgttaaacataattttactgtgggttctggtcaaaaaagatgaaatgtcattggttaacttgaaagagacattattctaaacattatcctccagaaaattgacatgtacaggaagcatattgaatgctcagcaaataatttgttttagagtaataatctttgagtattgtagtgattcgagttttggaaaagaaggcagtatcagtgttagagaataatattctttcaggattttatgtagttactttaaggtgcatgaaaggagactcagttgggtgttaaattcttgtccctgctttacttgaaactagtaatcttgagaaatttactaaatcattttgagccttgattaccttgcctgcaaagtgaggaacattttcaatttaaaattctatttcttttattagtattcttcctgaccttgaatcatttctattcccatcatgtccagttgccaagaaaaccatgcaacttaatgttgtttgttagccatcagattttattttactgaactcaagttcagtcacatcagttaccgataaaataataagttctatattaatatttgatatatcttgacattttgtattatggtaggaagaaataaaagaccttcctcaaattttaggatatgggcttctgtttgttgatgtttgtgttttatgatacactgttgtaaatcatactaaaggttttctctgaaaaagactagaattgacaatacttcatttgttagcagtttcaagttttagatcacgggtgcataatttcttgtgtgtgcacaacatgttggcattttatctgacttcaaagatgtccatatagataccagcaaaggataaaatgtaagaatgaatatcatgaaaagatgctgtatcagataaaattctagagcctaggttaaaaaacataaaggcaaacatgcttgatatataaaaattgagaaatatgataaaaagaagtaagtttaaagcatcagtaataccactgcctaagatgcagtttattttgaaatgttgtcttctagtcttcatacagtttaaaaatcatactataatcacaatttccagcattttccccttagcataatatattataacatccatgtttttaaaaatattttatgaacgtcatgtttagggattacaccatatatatcccatctcatagattcctttatattgaacatttggatttttatttacatttttatttacatttatttttatctttattttttgctttcgtggataaactgcagtgagcatcctctgtataaattttttttttctgttttaggattacttccttaggatagattgccagaagtggagttactgggtcagagggtatgaacttatgaactttttttttaaataaattttttgttcattttctgtccagtcatttgtaaaaaaattaaaataataatgattataattgttacctgctagaaaactgtgtctgaaggcaagattctgtgatcagaaaagtttgggaaatacccagggtaaaaagccttttaatctgatttacctcagcatttcccaaattaactgaacactgaaaattttttttgatacacttcttaaaattttgtggaagttttatataggatatagtctatgaactactgatgtaaaattaaaattgttttatttgcttgaaatattatgttttattggtcaaaggaattaaactataaatcttcatgctaaaaattattaaaagggattagtgttgtttactatggtttttgtgcatgtctttataaagtcttataacttttaaaattgaaatgttctatattgtttactaaagaacattttaatgccttggtggcatcaatacttttttgcaatcattaatgatatttagaaagtagacatataacatgaaagtagaacataatatagattgtacaaatcttgttttttaccctattttcccctgcagaatgtgaaaacctttgcatcttctgatagtctagccaaggtccaagaagtagcaagctggcttttggaaatgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaagtgagtatattcaatatattgttaacctgagaaactttacatatctattttaattgtaatgaaactttcctcacagtctttgtattactaaaaattaatcttaagatgtgtaataataaaatgaactagttttttgtcttacagaaggtaatggaaagtattacaagtagaaatactgataatgggacaatctgagtacaaatggagggaaagtaattgagcctattactatgaaaaattattaacatcaaattttcgtctaaaccttgcgtcccgggcagggattagtagcttgtgtggggtgtagaggtttcgggtgaaacatgatttgtgtaaatcaagtaatgaagaaactttttatttacaaaatgcagctgctagagtgggctattcacaagtgtcactctaaggcataaattactcagaagttgcctcaaattagttctagttatttatcatttgagaattagaaataatgaatattcatgtagagtagacttacaagttaaactatgttttggaaaggaacattttaacttgaatatacttgttttatagacattaacataatgtctgtggtagttcatcctcttactcctacttgcttatttcttacattttacagtgattttaggccataaacaatatacaaactgatttaacatcgtatcgtttaacctaacttgacttggaatgaagtatcttacacacttggattaataataattgaaatgctaaatcatgttttacatctgcttgtaaaaccttactttctaaaatgtgtttggccctaatttacctagcctatctgcaagtcagattttatttagctggcatgatctaagaaggacgtgacatcaagtagtgatttttcatccattgtaccatccctgtctcaagatgttgaagcagtagctttggatctcagccagatggctctaccactaacaactatagcaccactttgttaaggctcagaccttttatagtgtcattcctcccttttgcccccctagtcttgatcatcttcttcaaggttgctatatcaaaagggagttttaacatctctttttttcctgagggttcagttagaggaataggggtttaatacttaaggtccaattgcactaacttatttcagaaagaggttcaatcaaaactaagccttgcggaacaatgcagagttattgctgatttctgctacactttaaattaaaagcaaaaaaagctagttagacaaaacattatctttttccaaaaggaaaagtcatccttatattctgtaggaaaccttccagttcacaagtttgacaggtgctttatcttttctttaccttcaagatgtaaaattttgtgtatgatgattacctttttaaaaacgaagtatactgaatttgttagtttttttctagtgattctgaatcatgaataacaattattgtcctgtacatataacaagaatggactgggctctaaccatagaaggaatacatgtgtgtatgcatttatgtatatacacatgtacagtatagatgtgtgtatgcatttatgtatatacacatgtacagtatagatgtgtgtatgcatttattatataagtgtgtatgtatagtttttgatatattgttgatcatgagatgatctgccttcgtaagtatggcatgaagaatacaaagctatttgattgccaggacagtgatacccttctacaagtaagatttcagtctgaaatttttctaaatgttttcctttttgtccattcctgaaacccttctgccagagtattcccagagcactttcaacagcattaaaaaaagtcactgagtactgcttcacctaaaaagaaaatgtcagtattctccgtgtatacatgttttaatacaataaatagaagctctcactagttgaagtactttattttggaagagctttaaattcttgatcattataaagttaagacacttctacagatgttaccctagaccatagtttcattcaagtcttctttgacttttgagagtagaggcataagaaatgaaatcccattttaaatgttaacataggattagctttctacaaattttactcaggatacctttcaagttttattttttggcattctcataaacaaaccctgtggacagctagactgaattttacccatcatccctaaatatgctgcataagcaaatatattaatgagaaaaaaagatttttaattcttccttgcattgctttattctttgaaagcattctgaaaactgtcaacactctgaggacagtttttccccccttgtcactaaaggtttgttcttttgggagccttatggcaagcaatatatgttaaaaattccatgttgtcttaatgttaaaagtcaagggaagtggtagtcaagaattgctatttattaagctcacactgtttgaaagatcctactacagatacataaacaaaagttacagagaggccgaatgatccaccccagattacactacgtgatgagagattttaatcctgatttgcctaatagcaaaaaacccctttctctttccacatgctatgctgccccctatggttgaatttgacaaacatcactgaacaagatttccaacatagtcagagactctgccatgtgatgattttctgatactatgtcccatttgaagtgataagagctaccctatagaaaggacagagggctcagttaactacttttctttgtggcacttaacaaaacattattgaagattttacagctcaaggctaaagtaatcaggaacaaatgacgctgttaaaaaattttatagaggtcatactctcttgaatccataaaacatgcttttaaagcagaggttgtgtgctggctattcctctaaatcttgcttctccagatacaaatgtctgtctcttcagttttaactctcattctcaacatccttatcatatttttttaaaaaaagttgttttctgaagatatgcccagctcatattgaagaatctttgacatattttatgcagaatatcaactaattttctactaacatgaaaaacaaatgagatgattattaggccagatctggtatcctgatcttttgatgaagagaagtgtgtttgagcattaatttctgatgcttccatatctatcttagtggcttccacctgttctcttctcacataacttccttacctcaaattagtctatttttttcccatcttgattgttagattcttcctagtaattaaatcgtttttatccatttgcctcaaataagaaataaatgtttatcaaaaaataaatatatataaaagatagccaggtgtggtggctcacacctgtgatcccagcactttgagaggctgaggtgggcagattgcttgagaccagcctgggcaacatggcaaaaccctgtctctacaaaaaatacaaaaattagctgagtgtgatggcatgcgcctgtggttccagctactttgggggctgaggttggaggatcatttgggcccaggaggttgaggctgcagtgatctgggccacagagcaagaccctgtttcagaataaataaatagagggtggggggaggtgtggagaagcatctagtgtattggagtttagaggcgtaagaccctttcttgtctataaattctgaacaatagaggaatcaggtaataggatcattaaccaatcagggtatctggtccgccattctccaaccacctcctaggaccagagctgctgagcagctttgtaggataatgtggctaacattggaaatataacagggctctaagttacactctgttgtagagttctagagaatgatagcttcttcagaaagtccctggcctagtctacctgcaagacaagcaacttaagttcaaagtctgtttggtatatacatgtccagagagcaaaaaatgctatttaaggcagtcatcttgaataacatccactagtaaacatggcccaatgaatagccatgtcccctacctttgtgaaggaggaaaaatgaaacattgactcagcctaatttagcccaattttaaacatgtggggaggataatggaaatgggagaaatggggacaatgggtgagatgggttttcccctaacatattagagtgataagaacttttcacatctgagtttgaattctgattataccacttactgtataaccatgagtatataatctcagtgaacctattactttatctgtaaaataaagactcatgcctacctctcaaggtttttgtaaggattaagtgaaataatttatgtaaaacaagtagcatagtgcctgggacactcagatattttgcagatatcggccttgtgacctcattttttccgtctcctgtgccttaccccaaatcccacaaaataaacttcattccccgcagcggttgtcaatctctatatatatgttttacaaccttcagcattcagtcatcatatcaccctctagtgtaaaaagtagagaaataaattcttttaaaaaggtacataggaggattagaagagtactgtaataagctaggcccatcttcaataaaaatagacataaattttgaagtcgatgctacccacagatctccctagttgataaaatatgtaccctttcctttcattagaatttaagtcaatttctctagactttcttctaatctttaactaaagcttctgtatatttaaatgctttaatgaatcttccatatggatgccaaaatgaattttagctcatggttaatttctaattaatgcctgttaatatagttttaaatgtatttgttaatattattttaaacaataatagctctaaatgttggtgataaactgtatctcattcactgcaggtaatgacagttactattattaattatttaagagacggggccttgctgtgtttcccaggctgtccttgaacccctgggcataagagatcctcctgtgtagctgggactacaggcgtggaccaccgtgcccagctcaaaaagtattttaattatgaggtttcattgtagactaaagaataatcttcacaccaaatggaccattttaaaattgttttatagcattccattgattagttctttatacagatgataaacttcactgaagtatacacaaatatcacaaatagttatcttcaaaatgcttccaaagaaaattgtttcatatgtgtattataatgatctgatccatttttaatgttgtttttgttggtggtagggaagacagtatgaagaaaaaggacataggaatagatcatgatgaatattgttttcactttggtagttaactccaaaattatttctcaagctccgatcattcaccttagccatagttttgcatttctagctgtctgttgggcatcatcatctgagtggtccactgaaaactaaaagtactgtttctttctccaaaccaagtttttatttgcctccccaggttatcttttccctctcaataggtcccatcttctggtcagtggatagaaaagtggcttgactttttcttatcatctgagtctgggcctgctgaataatttaacagacttgatcacttagtctctctttttcttacccgccaggcaattgtaagataaaaattggagggtcagtgtttaaaaacgttaagagcatttttgcttgggcttctgtttgcatcaggctataacgccacgtccttgacaatgagttggcagtatgaataattggatgagttttttaaaatgaatttttagctctgtatagataataacctcaatacagatatctggtgccccatttcccactgataaagccaaacctaaatgagaatgtttgaagtcttccaatatcttaccatagtttttacctggtttcatctcctctcctctctacttgttttgaatcatactccttcccaccagccatatccttcaaatcgaagtccaaatcccctatcttaataccttcctagatttagaaaatcgaattcctttcaacctgttcttgcttctcttcatggtgcagttcatttggtattacctggtgacatttttggtaagctttgtcttaagttgacatataattcttcacttttttgagcagtagaaatttggtaaatttgttttcagtcctaattcagtttgagaattgaagactggcaaactgtaatgtgaacagttgtcatttcacctctttttttttttttttttttttttaagacaaggtcttgctttgttgcccaggctggatggagtgcagtggcataatcatggctcactgcagcctccacctcccaggcttaagcaattctcccacctcagcctcccgagtaactgggaccactcgtggcaccaccacgcttggctaatttttgaattatctgtaaagacaaggtcttactatgttgcccaccctggtctcaaactcctgagctctggtgatcttcctgcctgggcctcccaaagtgctgggattacaggtatgagccaccatgccccagcctcatttcacctcttaagatgtacctaagctagactagaaattgtaaatgggcacgtcattaaaattgagtttaagactcaggtattcagtatatgtacagatcgttaaattgagactgccctgaggagaagttaaaaatcccactttaggaatcattttgctaacatgcagaaatcagttgaaggcaattcatacagccattaaacatcacatttgaaagaatatttaataacaggaaatgcttaaaaatattttatttaaaatataggattcaaaactctatacagtgtgatcttaattatgaagcaaacatacatttttatatatgtgtgtgtgtatgtatctacatatatacatatgcaaagaaaaagatcagaagaagttgtaccaaatgttaggaattatctctgaatgtggttgaataaaaggttattttttcttaaatacattttataatttctccattttgttataacaagcatatatgcctctttatttgaaaaataactctctgctggaaaccaattattattctatgtggtcttttttgatgactttacatggtgtataatgctcctaatggaggattggataaaaagatgttcctcacaggaaatgtcattatatcatgatctctgcatgagaatgcagctgaatatagtggaaaccttcccctattcccaaccccccatttcatagtggaaagagcacagaactctttaagtcctagtgtaactgattcttcacatctctagggctcaggtgttaccctggcaattttctaagatcaacttaacacgttattagaagagtaatagcatagtagtatccgttcttgttttgaagatatttattgttttctttgaagagttctttagccttgatagcacttttggctctctctaaacatgaatatttttcatataaaaaccgttctccgagcaatttcagagtctgtttttgaaaatgggtgtccccaacccccaggccatggaccagtctgtggcctgttaggaactgggcctcacagcaggaggcgggcggtaggtgagtgagcattaccgcctgtgctccgcctcctgtcagatcagctgtggcattagattctaataggagcacgaaccctattgtgaactgtgcttgcgagagatctaggttgtatgctccttccgagagtctaatgcgtggtgagctgaggtggaacagtttcatcccaaaccatccccccacttcccctgtccatggaaaaattgtctttcatgaaacctgtctctggtgccaaaaaggttggtgactaccgttttaaaacacaattcttttttttttttttttgagatgaagtctggctctgttgcccaggctggagtgcagtggtgcaatctcagctcactgcagcctccacctcccaggttcaagcgattatcctgcctcagcctcctgagtagctgggattacaggtgtttgccaccacgtccagctaatttttgtatttttagtagagacagggtttcaccatgttggccaggctggcctcgaactcctgacctcaagtgatccacctgccttggcctctcaaagtgctgggattacaggcatgagccatcgtgcccagccttaaaacataattctaaagtggccactgctttatgaggaaatcatcagtgctttcccttccctcccttcccgtattcccaactccccatttcatcaaactaatgatatatttaacatgttaagggtttttagtcctgattttaagtctcctaagacagaccccaaattggagtggacctttgagatcaccaaggccaaacctgtgcccagtggctttaccaacagacttgaacttacaaatgaggtcattacaaaggaatactgcaatgtgaagtatcttcatagttttagacgttatttatgttagggttctttattttgtgttgacatctctgactttgctttggtccttgttcttctacctagagcagtgacagctcttttcataggagaagattgctgccatattcatctgtgatcttctccagactgaatgccttcaatgtgttcccagaatgtgtatatatacttatattttttctcatttatcttaacagtcctaatatgtcatttctaatctttaaccccataatggagatgagaaatatgtgaagagaggttaagtaattaatcttgtatcagtcagctggtaaatagtggagttgggattggaactcagatcttatgctgggtcccggttgtacttttgttaacgtttggaaattcagttaaacaatattagtaattttgggatgagtgggctgtgatgaaataacctataaagctaccgccctaacatttattttcatgtccccaaatgttgcctcaactaaatctctgttactgtggtatatttattgtgactcatcgtattaagtttgtttttctccctctctcttaagccctttgatttcttattttattgcctaataatatttattgtgttatcaggtaccctgataacctaattattagtcaacaaagaattagtgtataaatttttacataatacttgtcagtcattgaccacatggatatgtattttgtgtagctggagtcagtgtgtatacttcttttatattcctgattctttcagttattatgataataatagtcttattattatttggtaacctgtttgaacagttgacttgttcagcccctcaaagggaatttcccccactcccctctgcttcactgaaacatgacccattaacatgaattatttctacattccttcacctagtatttcatatgtatcttttttcttctatccctccaatctggtaagaagaaatacacttgtcctttacaaggaaaccttgtctttgaattattgatccttgaggtctgcgaaatgttttttggagacttcctgcattagccattctgtctttttaaattaacaagctttaatttttagattagttttagattcacagcaaagttgaatgagaggcctagagagttcccatatgccccctggtgcacacatgtacagcttcacccttaccagagtagtacatttgttatagtcagtgaatctacctttgtcatatcattatccagaatccatagtttccaccaaggtctcactcttggtgttgtgcactctgtgggtgtgggcaaatgtgtattgacacgcatctaccaatgtagtaccatagagaatggtttcactaccttaaaagtcctgtgttctgcctattcatccctttcttccccagcctctcatcgccactgatcctttcaccgtctccatagttttgctttttccagaatgtcatatagttggaattatacagtatgtagcctttcagattggcttctttcacttacgtaatatgcattgacttttccttcatgtatatttatagcttgatagctcatttccttctagcactgaataatattccattgtttggatataccacagtttatccacttccctagtgaaagacatcttggttgcttccacataattggcaattatgaatatagctgcttgtgaaaatctgtgtgcaggattttgcatggactaagtttttaactcctttgcgtaaatgccaaggagtgtgattccaggattgcaagttaacagtatgattaattttgtaagaaactgccaaatggcttccaaagtgtctatatacttttgcattcccaccaacagtgaatgagagttcctgtggctccacatcttcagtggcatatagtgttgtcagtgttctggattttgaccattctgataggtgtgtatggtatctctttgttaccatttgaagttccctagtgatatatgatgttgaccatcttttcctgtgcttacttcccatctgtaaatctcctttggtgaggtgtctgtccagatcttttgtccatgttttagtcaggttgttctgttactgttgtatattttggataacaatcgtttatcccatgtgcattttgcaaatactttcttccagtctgtggcttatgttcttattttcctgacagtgtctttcagagagctgacattcctattttagtagagtccagcttatgaattatttctttcatgtatcatgctgttagtattgtattgttgtatctaaaatgtcatttccggctgggtgccatggctcacacctataatcccagcactttggcaggctgaggtgggaggatcgcttgagcccaggagtttgagaccagcctgggcaacatactgagacctcctccttaaaaaaaaaaaatgcacaaaattagccaggcatggtggtgcgttcctgtgatcttagcaacccgagaggctgaggttggaggattgcttgagcctgggacatcaaggatgtggtgagccaagatcataccactctaccccagcaaaaagtcatgtctatacccaaggccatttaggttttctcttatgttatcttctaagagttttatagttctgcactttacatttatgtctatgatccattttgagttaattttgtgaaggttgtaaggcctttgtttagattcatttatttgcatgtggatgttcggtggttcagcaccatttgcggaaaagactagcttttctacattgtactgcctttgttccattgtcaaagatcactgtaccatattctggtctctctattctgttccattcatctacttttaattctttcaccaataccacactgtcttggttactgtagctttatagtaagccttgaagttggagagtgtctatctcccagcttcattcctctccttcagtatcgttttggctattctgggtcttttgcctctccatataagttttagaatcagttcgttgatatctgttacttgctgggattttgattggtattgctttgaaactatagatgagattggcaaggactgacatcttgacagtattgagtcttcctgtgcaagaacatggaatatatctccatttatttagttcttctttgattttttatcagagttttatagttttcattatgtagatcttatacatattttattagattcatccctaaatattttatttcagggggctgctaatgtaaatgatactgtatttgtcatttcagatttcactttttcattgctggtgtgtaggaaaataattgacttattatatattaatcttatatcttgcaaccttgctataattgcttttttttggtcaattttttaggattttctatgtagagggtcatgtcatgtgccaaaaaagacagtgttatttcctctttcttaatctctttatcttttacttccttttcttgtctttgtgctttaactaggacttcccctatgatgttgaaaagcagtggttaaagaggacattcttgccttgtgtctgatcttagtaggacagctttgagtttccatccttaagtgtgatgttagctgtaggctttttataggtagtctttatcaagttgagaaagttcctcccctctattcctagtttgctaagagtccttctatcctatttcttaatggtttagtagatgactctgtggtactttgaaggttgtttgcagaatttccatgccataggcaatttacctttccttgacatttgaaggattgatgttggtgccaagtatagaatcttcacagagtcctcctgtagcttctaaaggtttagcttgaaaatgttaattgcttaacgctagtaagtgagtgaaaaagctggggataaattttgtatcttgcttatatttcagttcccacctctgtcctgacaaaaccccaatatataaacagtaaatgtaagatatatatattttaaacataataacaaatgtaagttccttttgaaattattatttcaccaactttaaacagtattatcaatttttcaaacatttggccagtggttacaattttatcaatcttattagtgagattcttttctgcttgtgggtctacttttactttgactcttgtcctgaaatctcaattctatctttggcctctcttctgagagacataattttgtgtatttaattaatctctattttctataaattaatatgataaaaagaatcaagttggatggatcagaagtagaatacagagaaaagaaaatatggttagggaattgtgattaggtatgtttaaatacttagttcgtagacacgaccccattcttttgttttttctttgaaatttgccttagattcagtattgtctagttcttaaaatgctattttttttcatttagcattacaattatttgtgggaaaaattttaagaacatttcagaattctttcactggatattctaatatggatagtaattcaaaagaaagcacaaagatgtaaaatcacaaaagatataaaatatttaggtttttgtaaaaatcatatattagtatgtttagcatacatagaaaattttataaagtattatttttttttttactgtgaaacatcatatcaaaagcacacaattccttaagaatttatctgtttcttcactgaacactagagggccatatagcaccttatagatagagcggctttccatatatttaaatatatttaatatgtgcttgtctttctctcctactttgtgttttagtatagtatgtcttgctcagaatttggagtatacactcaacagtaattatgtgcactgtcagataatatgtgggatacctagggtttttgtttttgctttttttttaacaagcagctagcattttgctacttagtaacaacaaaaagagataaagagtgtatcttgggataaatgtatgtgcagacttactatgctttggaagtataagtatgcttttagaaaatttctcaagtatctgaacatatacgtgtaaaaagaataggcctctcattgttatttgctctggtacttcttggcatgtatgtgagaaaagaattatctctccaaagaagctacttcccttgtctaaatgtcatcttgtattagcacagacagtaatattgtggggtataaccacctaatcatcgtggagtaaagaatatttaaacaactgatttaatcttcctcaattttttttttttttttttttttttttttggcttcttagggtaaagaaatgagaaatacaacttacaggaacttaaataggtaatgtcaatgtgtgaagagagtcatgaacagccagtaagactgttcaggctcttatgaactacgtaagatatgccttccccaaaaggtgttaatgttcagttaaaagaataaaaatatgaggcaagaggatggtttgagcccaggagtttgaggctgcagtgatagtactacagtactctagcctcggtgacagagtgagactctgtctttagaaatatcaagaaaagaaaaagaaaaacattgtgctggctccaaaaaactgactttgagctacatatgacctatgggctgtatactgttggatctttaatagcttctagagaagaagttcaagatctaagtagggccttggctaaattttaatttcttccttagatatactttagacagcacttttcattattaatttctgtgttatgtttcagggggcagaaataaggacatttctcgttaactactgtttacactgtcttttggtgaaggaagattagtatgatgaacttagaggaaacacactgtttacacctagaggagaaaataaaagagggtcatgtgaaagagaaagaaggaacaataattcactcttaaagacttaaaagaatctcatcttttaagtctctcctcctttgatttgccaattttaataccttaaattatttcttcaaattagttactataatggagtgacaaaactttgatctttagttctaaattttaccgagatgttagtcctcttgaatattgatgaacttacaattattttaaagtcactgctatgataaacagtattctctaactgataatattttttatgccttagtggtgtgtttgctagttttctgtatcactgccattttctgaaaaccacatttattggcagattgacatgagcttaggtaattgaaggctgagcttgaattctgatccttttgcacaggtaatgaaatgagttattgttagcataatatagctggtgcatgttgaactcttaaatggtcaagaaggtaaatcaaataaaaagatcagtatttggatatgttgaaaaacacatggactccttctttgagtggcagagataatagatgctattgatggattgaagacatccattattgtgtctttaacacgtgtctggagaaaacactggcttgctagttaaaacttgattcatttaacagtattgctgagcactcaaagaattgccattatttttctaagatatgccacaatttaatagtagctcttgtagggaggggttggggaaggacgatgttgggagtagattactatgctaaatgcatatattcattgttagttgcattacattttaaaagggttaatgggagggggcaagtgcagaattgaggatatgtattattttataaagggtaagagatcacttttatccacccagaagtcagaggtttggaaaaagcaggttagtatgaatatgaatactagagatgtttctagaatatcatacacctgtattgtaactaatttccatcaatgatttatttactaacctggaatgattttcaagcagaaaaccctctggaccatagggtccagaaagttaattgatatgaatgaaatggactaaatataagagccagggagctaaaacttctctaaaaagagggtcttgatattgccatgtttttgaatttttcaaaattcattggaatttcagattttcagatgatatttcttaatctttaaaatatattggtgtccagattttgcaaaagcacttccgtgggttggtttgacccatagaccacctatttttcactagacatcatgtagggaagaagaatggactattattcttgaactgaataactgaaactgaaataaatgcaagggaagatgttctttggcacttctgtagaaatgtttatcacccattgcaaaattacgatagtttgcctccagtgctgatccattaatgggaattgggttgtttggagtggaagtgcggcatatgcatagcactttaagtgctttagccagtgacctacggacttttccttatgagtttggtgtactgattttagttattaaactgccaaatgaattgacctcattttaaagaaatgagtatccaacttccacaaggaatttgagatttgaagtggtctatatgtaggaaaaatacatataataaagtgacaaagtgtacatgaaaatcagaaccagggacaatactggagtagaatatgagggcaaggtcatgagaagaggtaaggattgagttatgatgagttggagaaagctagtaataggatataggttgtccactcttaagtgtctgcaccagtatttttcaaagtgttaccttggaccagctgcatcagaagcatctggagtgctttttaaaaataacagatttctgggttactctcaagagctacttaactaaactccagggtcgggatgtgcatttttaataagttctcaactgattttcatgtaaactgaagtttgcaaaacactggtgtgtagtttgttgtctctgctgccctgtgataaaattgacgatgtactgagtcctcaggggaagagaaaatatgtcacacacacacacttatgcacacccatgcatctttcctaatattacataaaaggaatttcccacataggacttcctgagtgactcagtagatcatgtcctcaacagtatagctataacaaatgcattaattctcatgaatagtggtacagtgtgatctctaaggtcaaactacggggggattttcaaatgctagctctgtcacttcatggcagtttgaccttgcacaaaccgttaaccttgtagagtttcttagggacggtagtagcatctaccacatagtgttattgagaggattcagtgaggtaatgtatgcaaagtactcaggacaatgactggcatatagcaaatccttaatgagcatttgctatggtatattataaggttaattattgtaatgttacaagtctatgctataactaactcactagttcaggctgggaggaatgctatgcgaaatatttttagttccaaagatgctcttttgggtaggttgaaacaattccattgacttttgcctacattattaatggcttttagcatatagttcagctgtttgatattgcacattttgttactgctaactatgaagcactcttgatccccgacagatattttttccctaattggttgttaatgctgaacttagaaggaatttgtaaaagaacctacaagtcaattttggtaatcttagagctttgagtactcatatgaaaatgttcagaagagaggaaaaggcaagttttattctttaatgatacttgggtgttcttgaactgaatagctgaaactggaataaatgcaagggaagatgttctttggcacttctgtagaaattctagaattactgcttgatactacagaaaatatctaagtttttcacagtgaagagatgattataaacaaatgaagcaatcactatgaatggaacaatagttttgggtaggttaaaattatctttaggatacattttagagaacacttcgtaaattattagggtaacttctagctgtatctgacataggatattaacttcattccttgtaataagtattgtcagggtagcatcttgcatataaactttaacctctaattatatgaaatgcaggaagaatgatgattgttttcatagatcaggtttaaaaatgatgtttaaaatttttttgtatctttccccctccccagaaaagggtctgttttattcaaacgtaccttgtccttctgtcagttggtttttgctgatgcaaaaggtagtttctctgctttagaaatagcagttgcttttaggagatgcattacttggttggtttccatggcagtttttggttattgaaatcagatgcaaatgaggaatggaagtatgaagaaggtactggtgaatttgaggagtaagtgccaacagttattcatttgcttagctacagatgttggttctattaatataagactacattataagtttaattttctagaatgaattgtttgtactttaccgatatgctgccatctgtcttagggtttaatgagtgaaaaacttaaatatactgaggtacctctgtagtgtttatcacgtgtttcagttttggggtttattcatcattatcattttactctgttacttggtatagaaaagaaaattaaaactttaagacagaatcattattctctatcagttagattatcactaatcttacattttattttaattatatttttgtgcttctttactcagaagggaacagaatagagattacaaaatggtgccttgtaggtatttaaatctttttcttttcatcatgacagcttaccattgaatgtatttttaaaacacatttaaatttgttgccaacattggaaaatcttgagagttcacataaaaatattgatttttttcaccttcttttgaaaatatggaacaaatggctttgctgggcacatgtacttacatggcaacatcaactgcaccttctaaaagggcttttcagatcttaaacctaatttgtttttctcctttattatacttgccttatttgtcatctgaatttgcttggaatatagacaaaagcatgattttaggaaatccaaaagtgagtttgatttttcctttcttggtcataacatcaaatgactttagatgtaaattcaagccaaagtaactctttaaaaagttttattatgaaaatagaaacttgctaaggcctcatttccattgagtgtcaaggtgggaaattgtcacccaaaattttgccatcctcaagatgatcacttaacgttttcattttgaatcacctgttgtcctttccattcctgttacattgtaaaattaacactccactaggatgactataaaccaacttagagctccttgttttataagatgtctcggttatccattgtcacctgaccaatcaccgtcaaagctttgtggattataacaaacgagaattttattatttctcattgttcttataggtttgtaatttgaggagggctgatctggccattgttaactctgggtggcagtcagatcatggctggaggtgcaaaagtgggggtctagaacagctgagggctgacccagaaattctttttccgtatcatctcctaacatctctatataatacggtctttttacctgggctagtttgggcttcttcagagcatggtggcctgaaggtactacttagccagcacatcaggactaccaagtgagtgttccagcaagcaaggtggaagctgaatctcctttttatgactcagcctgtgaagtcacatgataccatttttactatctctaatggtccaaacagtcacaaaggtctggcctgttattaaggcaagaggacatggcctctccctcttagtgaaaggagtctcagagtcaccttgcaagaagagcatgcaagatgggggacagcgttgtgactacctttgagaatacaatttgctataagagttcataatatctttgatgttattcctgtttctttttttaaatttgggcttcataaaaatcaggcacattaaaacattccatatttaatatgtaaatgtcttatacaatactttatttcagttatctagaactcatttatatctgacaaacttacatatctgaaacactgacaaagaagtgtctccgaacaactaatttaggaagtaacacaactaagttaggatgtaacattctgtggaattggatagatgttctgttacaaagcacaggtttaggaattagacataccagggttaaaatctcaactctactacctggcagctgtgttaccttgggcaaatttgtaagcctttctgagccttagttttgtctgtaacaagtagacaaggataatgatacctcagggtttcggcgtggtgattaagtgatacaaagcatgatctagtagaaactcttgtcagtgtttaatctcttccccttatcattgttccagtctgaaacaatgcgtacgtattttctaagccctatagctgcagacttcctttatatctcttactgatctctgatagtctccttagacttatttatctttctatcccaagatctttgtattacaggcatactaaagaatagtaggaattgaattttatttctttgtttctaatttttcttgatagccagttagtgttttacttactgtaaagaatgaaaacttaggctctcagcttaatttaaacctcagctacaaaaaataacattgatatttttcagaaaaattatttttttgagtatgtaatattgacagcgtaaaaggccagtggtatcaactgtcagcccgtccaagaattaagttctaaatatgtattttctattgcttaatcttatacttgttttctgtactttaattctatacttaaattctgtgctgaagtactttcagttaatattaataaatctcacatacttgcatagcacattactgaaaattatataaatgaagtatggtactatgtttatctttctcaaaatatattgatttttatttataattgcatcagaaactcaacagtataggaaataatgtccccctcctattttacctatttaaagcaccagcactccttcagattttttatgtttccataagtgtagctttttatttttcataagataaatttttataatttgtcaaactgtcaatttggttgagccttctgcattatagcttaattttttaaagctgtgtagcaaaaaaacacacaattacttactgtattttaaaagaatattagtattatttccttagtcctaattattagtataattagtattatgactccctggcctgcaatactcttaagaatttttataagagaaagatattattggtaagtaatgtttaatttattttaactgaaagtttaaactgttatgtttaaactgtttatggacttaagtactgacattaaagaagttattgtctttcttagacttaaattttattttgagaattatggaaaattaggttactttaaatcacttactctgtcattacatgaggctttttgcatgactgaaatacctgtaattactgtgtcatttttgtcatagttgacttaaaggtgtaactataataatcacgaactaaattcttggtgagcagctgtgtaaggccattttaacagctggggtaacccagctcagcctgtaagagttaagcttaatcatgagacaggttatgttaagtcattgaagacttcatactgggcatttctgaaatacttagaaaattcagtgggatagttggatttttgttttatcttaagtactttttaaaatgcttaatgaaaggatgaggaaaagtattttaacaactaaagttcttaggcacaaataatagaaataaaatccaaagtttactcattaaaaaaaaaaagtcaaatatactctatatgctagtgtttagaccactacagtaggtaggagaggaaaaaaatatataaacacacacacatattattatagtataatatggaatcagaaaaatcctgaggttatttttcttgaatttcatcttttaaaaatttttttaaaaatcattatgcctgtagcttctttccaataaaaatgttttcttattactatacaaaaacatttagagcacaggcatacatgccttttgtaaaagaaggcagacacaaaagaattgaaacaatgcattttatttggatatattttataaactagctatattgttcactaaactataataaaggaaagctcaccaaattgaaccttcttacaagaaggccatatatgtaatattattagagctgtaatatgcttttttttggtataaatttaaggggtactctagttttttttacatggatatattacatagtggtgaaatttgggcctttagtgtacctgtcacccaaatagtatacattgtacccgttaagtgatttctcatctctcagcatcctccctgcatgtcacccttctgagtctcttaatatctattactacacactccatgttcatgtgtacactttacttagctttcacttgtaagtgaaacgtggtatttgactttctgtttctgtgttgtttcactgaagaatcacctccaattccatccatgttgctgcaaaagacatttcattcttttttatagctgaatagtattccatttgtgtgtgtgtgtaaacatatatatatgtgggttagttatcatagttgacttaaagttataactattataatcaccaactaaattcttgggcatctttaaaataatgatttcttttcttttgggtagatgcccagtagtgggattgctggatcaaatgatagttctattattagttctctgagaaatctccataatgttttccatagagattgtactaatttgcattcccaccaacaccgtataagcatttctttttctccacatcctcgccaacatctattattttttgtctttttaataatagccattctgaatggtgtgagatgagatctcattgtggttttactttgcatttctttgataatgatgttgaacattttttcatgcttgtaggcatgaaaaaatatgtatgtcttcttttgagaagtatttattcatgttcttttcccactttttaatgaagttaatttggtttttgttgttgagttgcctgtcaattctgcatatcagccccctgttagaagcatagtttgcaaatattttctcccattctgtaggttgtcttttcattctgttgatttagagatgtaatgtactcttaaacctgcaagggacttaatctcaatcatctgaagagaagtcagatagtctatatctattaaatatttagcctcctctttgtagaaatctgaagtataaactccaggtaattgtactgcaagacattaaatagttaaggttcctgaatcttggagtttctggtgtaccatgggatggttttgtcatctgtcaacttataatttattagcacttgttttggttggttttctgtgttttactttttaatttataatgtattttcagcaaaatttttggaaataactggaatataaaacagcttaaatgtctaacattgagatgttgattccataaatcagagcagttacaataccttatataatctagtattctggcatttttattttgagaatgctacctatctgtgcatataggcatgtacatgcaatgcttttattttgcaaatgtccaattatcaggtcacatttttataacacttgtgtatgttgtatgtgctgcttcagaacccaagcatatttctcttagttaggggccgcccttgttgcccaaatgaagaaaattagcagggaagtgcagtatgttgtccattgaatgtttacatacatgtaatgtctcaaatacattataattggaagttgtaatctgagtgagcactttgagcatgtaaataaatatcttttagaacatttttagtaatcattttaaatgtgattttaatcttataaaaacattttaatttatttgacataccttttgtgaatactaaagcttaaaaaaaaaatactaaaactttaaaagatatcctgaccaggcgctgtggctcacgcctgtaatcccagcactttgggaggccgaggtgggcggatcacaaggtcaggagttcgagaccagcctggccaacatagtaaaaccctgtctctactaaaaataacaaaaattagctgggcgtggtggtgggcacctgtaatcccagctgctcaggagactgaggcaggagaattgcttgaacctgggaggcggaggttgcagtgagccgacattacaccactgcactccagcctaggcaacagagcaagactcagtctcaaaaaaaaaaaaaaaaaaggtatgccatgttttttttctttgatgcccaatctaattgtatttatgtttctgcatttaaagtaatatgttgcttgctggatgtttttttttttttttttacagtttttgctaatacaagcttgataatttatcattaagcagagggtttaatttattgatttatgattgttttcctctgtgtttaaattgttccgtcttcaaccatgaattgcgttcatttgtgaattgcttggtagggaaatactttaatactctgatatctatgatgttaggaagactaaaaactgatttggtagataaaataatctgctttactctgtatctgtgaaataagtgagtatgcttgataataccatattatctgatcagaaggattttgattctgatttattgcacttcagtaatggtatactttttagcatgtgggactttctgtttctacaacagagtaagtcacttgttgcctgaattcacagaagcagtgaataatactgaattcaaataacatacgttgatattattcctgtgaaagtttttgaggtaagactttgaaaactcttggtataaagtcaatttgaatttgcagggagatttacatcttctgagtgatttgttttagctgattcaggaataatttatgaaaaatggaaaatctttgtgctgctcatatgctgtggctttggtcaggagcagttaaatttctagccttttgtataatactgaatgaaagaaaagatggaaatccagagtaaattgctgtgatggtatttttattgttgttacaagagaattggagtgcttggacttgtaatgcattttacagataccgtattggtgatattttattacatgtcacatttatatataattgtttttacggatgcaaatttttagaagaaagaaaatcagggagtgataagtgggcaaaataagttaccttatataaagaccttttctttactacaagttccctaatgtttagcccatcttggatgatcagtggtatctggaatcttaaaatgccaagacactatggctaagatactacagcttcatattttggcaaaaattttgacagaattatagaagtctttctgaaacttacaaataatatgttttaaaatattcttttccaaagttattatagatggattttagaatctgctttatttcctggtgattttaaaattggaaatgtaatctacaaagctctatgtgatttttgattccattaatagtagtagtaatcataatgacagtttaattaacactatcgaacacttactagatgtgaggcgttactctgtatacttatatcaactaatttttaatcctcacaacagtcctatgagtactgcctaggtgaacttgagacacgaattaaattaaggtcatacctggtaaagccaggatttggactaagttagtttggttctggagtccatgttcctacctatcatattgcactgccttattatttgtggtatcatctttaaaagagcttgtctgtttttaagagtctcaatttgggtagtaattaaagagtatctttaatgtttttaagagtagaattgagactcagactttatcttttcttgaacacatggaattagctacattttgaagtattaacagttggggttcttatataccttgtctatgatttccctcatttattagggtttttgtctatatttgaaggtttgtttttttagtgtttcgtgttgattgggaatatcgcattagaaatactgttacttgaatcctcccaaaaaaatgaaacttgtagtatatatatttggaaattagtactcactttgcccaatcattgataatgtcttctttttccaaaaatatattatgacaaacacaggattctgttacagcgtttggtttgccacagttgcaatgccctgtattatccagttgagggcagctatttactgatttaaaaaaataataattgaattagaatgttacccccttgtgcttaccaaaacttggtatccactaaaaaactccacatttgaggtacaaacagccttaattgtgttcacttatgttttgtttctgttgtcattgtaaattatttcatgaaaatgaaaagcctagctttacttcatagtaacttttggtctcttagaaactagaaactgttgattttagttttaattttcccaatccaagttcttttcaattctaaatcttttttccccccgttaggatcagactcattgagatcagggattttcttatctttaaaatgtctgcatgtaaatacatgtgactttagagttctttgagggcttttaaatgtaaaagtgaaaatttataatattgaatacattatctttattaaccagtggaataaccactgatggctaactaggtctttgattcatttttctaaatgccaaatgaacatgttaaactcgtagaatatattaccctcttctaatttttctgcattggagtatcacattgttaaaattgtatcttctatgtagttcaggaaaagaaattcaggggggttaattatccaattgatttcaaaggtatcactgagacagaatttttttgaaaataatctcagactgcagaaaactgttgtaatattaacaggtagtgttagtaactgagataaggatattccatgggacctactttagcaaaagggattgtctaatgagagaaggacaaaatatgacatatgtttcacaaacaaaacaattagtcttatgttggaaaataaagtgaacatctgaggagatgctaagactagcatgaacctagtattcattctgcaggatagtaagttgtctcatagggctgtagacatggtatcttggatatcattctcactttttttctactaccagtttataggtttccaacactctcatattatctcttctctctcctaaccttagatctctcgaggtttttttggtgatgattattccattttcaatcatttacttttttgtttaaaaacatttaaaatttatggttaaatatccataacatgaaatttaatattttaaccatgtttaagtatacaattcagtggcactaaatatatttacattgttatacaaccattaccactatccatcttcagaatattttcattttctcctactgaaactctgtgccaactaaacactaactctcatttccccctctccctgtctcagacaaccatgattctgttttctatctatgcaaatttaagtattccaagtaccttatataagtggatcatacactatccttttttgactgacttattttacttagtatagtatcttcaaggttcatgcatgttgtagcatatgtcagaatttccttctttttcagagctgaataatgttcccttgtttgtatatgtcacattttgtttattcattcattcattgagacttgagttgcttctaccttttggctattgttaataaggctgctataagcatgtgtatacaggtatctgttcaagtctctgcttttaattatttaaggtatatactcagaagtggaaatgctggatcatgtgctgattttatgtttaaattgttgaggaactgccatactattttctacagcagctacattcattcatttcacattgccgccaaggatgcacaagggttctgatttccccacatcctagcccatgcttgttgtttactgttttgttttgttttaaaaccatcctaatggatgtgaagtggtagcgcattgtggctttgatttgcatttccctaattagtgatgttgagcatcttttcatgtgcctattgaccatttacatatctttggagaaatctgtttttgtgtagcattgctgccagtgattttgcatttttaactacattttaaattgtggtataagagaaaggtctactaataggaatgggactatttcttgtgcatagattgccttcccagcagcccactttaatatttagcaatgctaatctttccaaagtcaaaaagaaatcacccatttctaactttctcctttcaaatcctttcttgtctccgtcaaaattcctgctataaaactccataaaagcttggaagttaaaagttggaaggagagataagtggagctgactggctgttggaagaagaaaatagaatggaaagagagaatagttgattgagcactgtgaatggatagttcacaatcattatctactatctgttaggaatatggattatagtataaagattaattaacacttgcacaaatggttaatttagggaaatatttgagataatgctggaaagtttagtttggggccactttgaagagagtcttcatctccagactttatcttaggccgtggagaaccattgaaggttcttgaggagtaaacggcacactgcattccctctctgtgttttaggaagatttatctgaccatactgtgaaggagaaattaggcactagggatttgcaatggactggctagttactctcccattttactataattacttcccatcatagattactttataaataaatacactttttacatgtctttaatcagttgggtgactgttatgctttcaagtataatcaataagtttttcagaattaaagattgacaattgaaaactctggtacaaagattaatctgaagtgtgtattaatagctattagagaaccagagtatacagtatttaatgtctagtgagtatgagataaagaatggtgtgtatggaaaagggaaagagagcacacacttgaaaatagtgctcattataatatatctaaggtagaacaaacatacatatgtgtgtgtgttttttttaaaccatacgtaaacacagttcaaaaaattcaaaacaaaatttgtaacagaattcaaaacattcctaaaatattaatgctgacatttggataaggaagacttaatagatactcaagatacaatgtttgttttttttattttatttttgtaagtaaaaatttactttcactttctaggaaacactcttagattagttagttggagaaatattaaaagggaccttacagcacagccctgctttggttgtgttcacattactgactttgtatgagaggttcaccagctgtttgactggcaggagcaggagctttcacttcagtagtttataattcctggctattctaggatagtttgcacttcaccaagcctcagacaggtcaggacatttggtaggggaaggttgaaagacaaaagcagcaggccttgggttctcagccttttaaaaactattattaaatatatatttttaaaatttagtggttagagcttttagtaatgtgcctgtattacatgtagagagtattcgtcaaccaagaggagttttaaaatgtcaaaaccgggaaaacctactctaaaccatggcttggttcctgttgatcttaaaagtgcaaaagagcctctaccacatcaaactgtgatgaagatatttagcattagcatcattgcccaaggcctccctttttgtcgaagacgggtaagtctcacacatttgaaaaatatgaatgtttatttaagtgaagtaccttggcctcttttagtgaaaagatgcctcaaatttcagtttttatttaaaattaaaactatcaatgcattgtgtagccagtaaacacttatataagtgatagttttataatatgatatagagcctagaagatagggttacccagttcacctttttctctcatttatttttcatgaaattagaatcctgcagatttatctaaaattagtaaatgtaaaaaggtatgttttgaaatacgttgacaaatattagcactgtaaatttgtagtttttgcatgaaaggcaaaaagttgatggtattctccttatacatgggaaatacagcatttcattcgttaaaaagtgttccaaatccttaggcttctttggcctcttaagctgtattatttttttaaattgctattccttacattcaaacttaataggaacactactttttgctgttatataaaataaaaatagcatgctattgtgtggcagttacgatatggtttatatatagacaatcctgaactaacaaacattttgtagaacttgtatgatctattggatgtttctcaggattacgtactcctctccctggtcaacaagttttaaccctgttaacatgagcaattttttgtatttcctgagttaatttaaattgccagtttcaaattatttattagagtgatatatttttagatctaacataggtttatgagcctgaaacaaacaaacaaaccactgcttgggattaaatgagtgtatttgtgtatatcaacactgttagggttctatctttttgtgcgtttcttaaagttttattttgtttttaattgacacataattgtattgtactttttcttttagctaaaggggaaatattctattaaatagaataatccataacactggaagaatatttttcctccttaataaagagaatcttctatttgctggaacttaaccttcataaaaacatattttctagttagcttagttctttttgagtagtttccacttctacgccaaacaagtggcagccatgttggaaaacttttacatgaggttaaaatgtaatacagtttaaacatcgaaaacagaatacaaatatccccagcaccctcttgttacttttgtactttactgttttaagaaagtgtttgttttattttgtctgctttcaccttgtcattttagtagcttcttctgaatgaggtcactgctctgatttgagtattttatagaggcctctaatagctttaaagtcactaataaactaatttattatcttatacccctgtaaaagaaaataccaatacaaataaaattaaaaattgagaaaaagaagtgtagggctttttaaaattaaagggttaagatgttaataatcaagttaatttttttattgaatatttttaataaacaagtacatatgggagattgcctcattatgttacagtatatatttcctgtctttgtcttctaacatggacattaagtttaaaagacataggagaagattctgtaagataaattaatagagaactgtaaggctttcctgagggctttctggggtcactatttaatttgacttttctaggttactgtgttatgttaaattatagccttgcagttgtaatagcagaataaaactaaatattaaaaatgaaattcatattaaaatttttagaagatatttaaaaaacaaaatgaacacattcaattttagcctgccggccaataatttactattgcataactcattgactctgactactagaaatattagagggattgtgaaattatatcctttctttccttttgcaaatggttagttgatttacattttctgtgttatttattaactttcccatttttctttattagaaattttaaagatatttttaatatgttataattaataatattttgtacctgacgtattgtatggcttttacatcgggtatttttactttctttcaagcagattgctatacaccttgacaacaaagcacacaaagcaaacattcttctgtattttacagaccacttttttccttctaagtcatgagctgaatgtaatgtatagttattgcttatgagactatatgcagtcttgatctattaaaatttgaaagcatggtattttccttccagtcatcatagatactgagtcaagttaatgtttgcacagttaatattttggacagaataaaaaaagtttgaaaatagtggaaaaaattttaaggtagcagtaataactataacttcaatttatatctcttcaaatattttcaggaaattaatttaccttactattaagtgtaatattagtactggtagttaatgtattatataaattattctagctgctcatctgagataatttataagcattggatttaatactaacaacagtaaataaatagcaacctagctattaatcatttaccttcaaatgaaaatgtttttaggccaggcatggtgggtcacgcctgtaatcccaacactttgggggccaaggtaggaggatttcttgaacccaggagttagaggttgcagtgagctgtgatggcaccactgcactccagcttgggtgacagagtgagaccttgcctctgaaaaaaaactaaaattagaagagagaaagaaaaaaatgtttttagtcttgcagtcctcataatttgtttgtgttttctcagattttcctggtcataaatatcagctggaatacttactaaaaataagttctgcaggtatttcttagtatcagaaaaatttgggaatacaatttttaactaaaaaaagccttggttgtcttttaagggaagataaattttacaggaaaaaattatttaacctataaaataattaggtaaagatatttaaaataaccagttattttgtaggttatagaaaaataactattttttatttgaaattggaatgcctaattatggtaatgttatatttcttaaatcctaagatgcattattttttcacattataagcatatatgaaataaggatacatcttgatggcactagatagcttacctagcaacatttttttctttctagtaataagagaaattatattaagatgcagcttacaactaataattgcatggttttttgttttgctttttttgggacagtgtctcactctgtctcccaggctggagtgtagtggcgccatcttggcttactgcagccttcatctcctgggtgcaagagatcctcccacctcagcctcccaagtagctgggactacaggtgcatgccaccgcatctggctaatttttgtatttttgtagagatggggtttcaccatgttgcccaggctggtcttgaactcctggactcaaatgttttatctgctcgcctcagcctccaaaagtgctgggactacaggcatgaaccaccatgcccggcataataagtgtatgttgaaagaaaatggacttttaatgcataaatttcatctcttgactcattttgtttttgtttggagcacttttaagcatttagaatattctcttaaaattattcttcagtgttactattttagtagaaatttccaaatgacatgcaattgttggtttatttgggacttaaatatatctggtttgactgctggtttatagacaaatattgtatttagagtgcctcctatattcttgagtttgtgtagcataggcatcagcaacacaggagagaatggagtagagctttggtggaaaggatggagcacagataagggacaaggatttatgcatctgagacccaggaactcaggaaatagtgaaatatacaactgacgtttccagtagttgaaaataatttcatagtgattatattctatatcaaattctattccaaatactgtacttaaaattatttttctttaaaataaaaatagaaataaaagtagaatatttgtgtgtcatttgaaaaggtgttactgtcacatcttatgtatcgtcatcttaaatcaggagtccacatgctttttctgcaaagggccagtaaatattttaggctttgtggaccgtgtagttggcctctgttgcactcagctctctctgcctttctatcatgaaagcagccacagacagtacttaaacaaataggcatgactgtgttttaataaaatgttatttataaaaacagtgaaaaacaggtttttactgttacaacagtactgtagtaaattttcttttgcatatagctttttgcttgtgtaagtttcattttatggtgagtttttagaatggggttctggagctttaagccattactttcctttataagactcattttctctagttcctttgtgtttgtgggaggaagaattttatttttgcgtatctaatcactctgattttttttttttttttttgagacagactcttcctctgtcacctaggctggtgcgatcttagcttactgcaaccttctccttccaggttcaagcgattatcctgccgcagcctcctgagtagctgggactataggtgcccaccaccatgcccagctaacttttttttttttttttgatatttttagtagagacagggctccaccatgttggccaggctggtctcgaactcctgaccgcagttgatccacctgcctcggtctcccagagggctgggattacaggcagagccaccgtgcctggcctaatcactagaattttaacatactgctttttattatttatcttctaaatgtataataatggtattgatgatttctatcattttaaaagagccaactatgtattctaggtactcctcagctactttttatattttaatccatttaatccttaaatagtttaaatgttaatgaaacaatgtaagttttatctccattcagtagatgagaaaactgaggctcagacaagtgaataactctcccagttttccaggtagttcggaacgctggtatttaatctcaggactctctaatgctgcaggtactggctgttcttttactatgctttgtaacatatacattttttttcttgtgaacttaacacgtttcaactattagtattttgtagggttagtctgggggcctattgctgttgacattgtttctatgagacaatacaaagtttcaaacagttgacttacaaataatttttgtagcaccgtccctttataaatttaaaattttttgtgtaatcccttttaagttttttttttcctttttagttatagagttatctcacagattatgtaacaattttagtcatgaaaaaactggaaatattcagaccgtatgcatgtttgtcctgtgtctttttgtaaactgagggatgatacttcagggtctggggtcaggcaactgagaacagggtgcctaacaggtgactatcctcaaaagacaatgcagtaccattctcatcagttaatcaattagtacttttgacaattatgaccaaaaaaaaacacagcacctctcataggacctcttagcaatactgtgttaggttcaggtttttcttcattgttcagttattcatgataatagacaagcctatattcaaaattttaatcttctcattctcataacaaattagtccataacctaatttgttgtttttgtatgtataaacttcagttagttcaaggggtgtataattttaatctattatactcctgtagtagactgttgggtttgttagaacttttcagcaaggtctgctaggcttattaaacagtcgtattgcttttgtttcagaaaataacatctctaattatttattctacaataaaattttcattaatatgaagattttatacatacccaaaacaccgaccatcataagctctgttggtaaagcaaatttgggaattattaccctcccaaatttagtaatttaatatcctccaaagtctgagttcaatggctatataatgaatttttatttccctccaatatttaatatcttttacttctctttataaaactattagacatgttaaatatgtaatgtagaaaaggcacttgtctttacagtgtcctaaattattagtaatctcacgggtcagaaataaccacaataaggattttaagcatataattttttaaacaaaactgccactttaattcatttttaatatactgctagtattttcccatggctgtaggtattcaaaagtatataatctgccatataaatatatcgtaatttctcactattgttgatttagattgtgacctgctttgaacatcgttatgcataaatttaaccacatcactgtacacagatttctacaagtagttttattgggtcaaggagtgtgaacttctttagattcttgataaatattattaaattttttcagaaaaaattgtatcagttcaaaaacagcatggggtttctaatgttacaagacattatattttaaagcacatttattcttatggcaaagctttgtggactttacttagctgatactgtatcattgtacttccatgtttattattctaggttttctgtgaactgaattttcattttgagaaaataacgatatatgtttttacttaaggcctcataaggaaaatatatcaattgattaaaaaattatttgtcgctcatagtgattttattttttattcttataggagataatatttgccatagacttgtcacctatattattttcaactgtaattttcttctattcttttcatcgttttaaaactgtgatctgtgtttttatctaaatagatgacccacatgtatgatttgataatttattattgattcaacatattttttatatggagagtcttatggttgtctcattttttgtcacattagattatacttttacatttctgaattttagcatggtatctttacagtataagtgacaacctctcccaaagtatatagctattaataatctctactgtaagagtacttctcatgctagcaggagcagctgccagaaatacttgcctgtttcctatagcactgttacttagcattacatttctgtcatagctttgtcatttagccccaatcctggtttgccttaaaaaaaaaaattctagtctagctatgattaattctcatttaaagtagttgtagcaagtctcctcttctaaaatgtcttgatgactaaaagggatttcacgaaactcaacatcactttcatagaactcttagccaagtaaaaatatattgatacctccttaacatacggaaatataaaacgttttaaaagccagcatcatccccccccctcctgtaactagtctatttggtatccactggttacatttgtgcctgtttttctacatcctttccatgacctcatggtccagcttacctctctcttgaaacctcccttctggcactggtttcatccactgttgtcctcaattattgctgttatctttggtccttaggcatactatatgggctgctcaatgatgttatatgacaactttgtatagatatctccttggttaggttataaactcttacaattcagaggttatttttaatacctttttatgcttccagagatatgcaggagaaagttagtataaatatttctatggctgattcattgaagtgtctgttaaacttggaaaaatccttaaggaatttttgtgctgtaagaatgtggcttgtttttgtttactaaatgtttttaagtgactgtgcagctgtgattttgaatatttcattttctaaatttaacgaaactcagatacagtttgacacttaattttaaaagtaattactatgtactaattttgaaaatgaaaaagaaacacacatggtatagtcatttaggtggtacttttgggagtctagagtagctcctaaatatcagaaaatgctgaaatagagaaaaaaaatacagtactttgaagagaagaaagaatagggtggtggtatgagcaaatttagagagggaatttaatagggtacctgttttgtggtggggtttttttggtttgttttgtttttgtttgtttgtttttgagtgaggaaactaaaatgtggctttctaagcttctcttacttgcatatatcttccattggtacttggaaaatataaacatttccagatcctttggtgttttttatggtcactacttactttatcaataaaggcattaattactgtcagttatcacatatcagaccattctattttagatcctttggtctttttctctttaagaatgtttcacacatcatgcatcaacatgagtaagcaatgtaattgtcggttttctttgcattctctgtgctgtgcaagccaccggctataaataagatgatgatcccccatgcccatgcacaaatgaaaagtcacagtgataaaactacaaacaaaccattatagtacaaaataataactgcttaaatggacctacgtacaaagtactgtcagagtatgaggaggcactcagagctcaatatgagtggttcaggaagggcttcacagtaaccctgaattaaaacatgaagatgtttgctaagtaggtgatcagaggagtgatagagggaacttgtatcattgcttgggatcgtgtcttgtcttatgttttttcatattaagtatactgctgcaaattacatcttgccgtcaatttattaatgttgaaaaaatacgtattgagcacctagagtagagaactttcctagaaactgtagatataacagtgaacttgatagacaaaggccatgccctcatggagcttagattgtagtttggggagacagaaaataaatactactgttaatattttgatgacagtaaaatagggtaatagaataaggtatgatttgggatgtagaagaggcactactctagttactttctatacataaactcatttaatccttaaaatcaccttgtgaagtacgttgtattgtattttacctgttttacacgtgaggaatctcaggcataggaaggttcttgccccttagctagtaagtaatgtagctaggacactaactcaagcagtcttattccagcatcacagccactgcaggaaacggatctggagtaggcatttgttgaacagacccttagtgtccaaatgttttaagggttaacttttgccatataatcttggaattttaaatgtgtaattctcacctttcccaacaatttatgatggtctcaatatctaattactgataatactgccttctgtatgtttttgtcatcagatgatgttttagtattgggttgtgccctagttgtcagataggagtacaaagcgacttagcacatgatttataaatctgtaactacactatggaaacaacttgtatgattttgtcttttatgtaacaaatttttaattaggtaacaatttgtataattttgacatcaaagggcaaggctatttaaatttgtaaatataaactaagttaggttttacagaaactaacttaatgttgaggcttgtaatatagaagacagatgaaaaatatgctaaaaataaacttggaactaagctcctggcaaggtaaacaagtgtgaaataagtccacagtcccttgtctgcaaagcagaaatccatcatgctctgaaaactgaaaatttaaaaataaaggtagcaaaacctaacccaaactaacataaaacagtcatctttattatttatctgaatattcatatattttgctggagaaacactaatgtgtgatatactggaagtgctctgtaataaatagtatatgcactctgttacctttctacaattcaaaatattctgaatttaggaacacagctgtctccaagagcatggaactaaatccagagaccttagagttttacaactgtcacccactcataaaaaactgaacacactttgaaatgtcagagtagtctaagctttatcttaagtctctgagtacatgcatattaaatactttaaaacatacatatatagatgagtacctaaagtttattaacctaattataggctattataaccatataatacaatgatagtttggagcttttatattacttttcttcagtgattaaaatacagctcatgctaatagacttattctttaaatgagtgtagagaacctcaaccacattttttttaattgtcccagtatttccatgggaaattaacaccacaaaattaattttaatctgctgatatatagtatgcatactctctgtgtgtgttgctactgttacaaagatgaataagacagcacacctttcccttaaggaatccacaaatgccttcatgtctctgagagacttgtctcatcatccacatttcatggtcacatatcaaaattagatgaacatggcttaattaatttattcaacaaataagcatctgctaatagccaggcccagttctaggcacttgggatacattagtgaacaaaacaggaaaaaaagtcctttcaacattttagcggtcctgagaagtcctccaaggcaagaggcagtttgtgccaacatacagtgtgtcatcagactcctttattaaattatacacttttcatttctaaatcatttggtgataatttgtattatagttttttcatatgtatacataactacatacatgtacatacaatttacctgatttgatagaattaagttagtggccttattgaaatgtataatctgccggttttcaaatactgttagatagtatctattacaagcaaaatattattttgatgctctgaaatgataaataggattccatttaactccctttttaaatatttttgtgaaacaactttcttttggtgtttttaataattctatcaggcacagcttagtgatttctttctgaattattcctggatcagaattagacttcttgtctttttccatgccactatggaaggttgcagaaactagcatgtatctcacatataaattagttatataatggtagataaaatttatggtagatatatttgtgttatattttggtataaataacagtagatataatttaagatttttaaagatgatattaattttactatagaaataatagtggaaaggcccagcatttttggtaaagttgatgacattctaaatgttagcagtttcaattcagttatttcctttcacaagtacctgctgatgcagcacgtctctttatttgtatatatagtgttttactcctaaaatatgaagggtaaaattattaaagtagggtttttttaagtcaatatcttagattaataaagttttcttttgtgactttgatagtgtcttgtgtttatatatttataaacttcattgcaatttaggaatgggggaagcagtgagtctttctcaaatattaatactgtttttgtaattctgtttttctatactgaatatttttcagagctatcgaaccataaaatagcaggcttttcttcttaggagagccatcatttctcaaacgtacatcttactacattggcattatttgcatatttgtttttctttgagagagagaatagttgaaagtggccattgaaagtagcacctccaagtgtagataatgctaaaaacattgcagtgatccactatacatgtaatattttgatataattaacttgaaacattaattttatagtggaattccatgtttgcattcattaggaatttactcacaactaaatcaagacttttaatttgaactttcttcctcaagtaaaggtagcactccccaccccatacataaaagagacagaaaaggttaaatatttatcccaaaagcacatggctaattagccatttgtttatgattattttgtggtatcctcatataatgatgggaaaatctgttttgaggatttgagaaaatttatggtaatgttgaatgggattttcttaaatttcttctcctttgctagtcttgaacatgaaaattgaaacttatgagctatttaattacatttttatgtgtgggtgatttcatatagttcacttgggaacaatttcagcttaaacagcttttcaataatccatagaaatctctgcgttatcactttagattttaaaatgtcactgttgctactaatatgaacatgtaggggcagtatagtactgtttttgtaaatcttgtctgaatttgtgagttttaaaaatctgtgttactgttagaagtttgttcttctctaagataacataactatatcttgcttttttaaaggatggttggtactgcttcattaagttggaactattctttcagttcatgttcttacatgtgttatacaaaatctcttatattatgaaagctcttggccctgatttatatttgaagtcaaggaatatgtttagttaaaaggaaaaatgcatttttattgtctgacaataaaaatgagaataaatggattcataaaaaccggaaaataccaagatcagtgcagttttgcccttactctgtgtcatcctttcaaaatgaatgtttatgataatttttacagtcttaatttttcaatatgacaagtgaacaaaactgagattaccgaacagtcttatttttaagattctttaattggtacatctttttttcgtgtcaaaatggttaaggttattacaaactttattacaaaatatagtaacttttcccccgacttaatatttaaaagaagacaactgagcagttatctcttccatctatgtattcactttattcaacacctcctatgagccacggactgctttaggcatttagagtacaacagtgaacaaaacatcccagccatcaccctcgtggagttcatatcagaaaagcagagtgcctaagagcgcggattatagggtcagttggacctgggtttaagttctttgctgtaccacttactaagccttatctgtgaagagctaatagtagtaccttttacagagggttgttgtaaggataaaatgaaataacccatggaaagcatggttatgtacaagagttctgtaactattatgaagccctcataagacatttttagatgtgttggtaaaatttataattaattctcctgatgtaatagtacttatgatagagattttgagtatttttttcatttttagtttcttcaagtggtaaaagaagatgcacagcataaattttgaataatttttcttaaacagtttcctgagtgacttttaatcttctctgagtggaaagataccatgcataaaaagttaatgtgatttggttctacttacattatgtagattcattcagtgcgactatcatatatgtaaaaattaaccccattatgtctccctacatgaagaaaagcatatgtgacatcttggtagtttgtttggagattctcagatgttcagaataaatgtgttggtttcaaatgtcattaaattggaatttttaaaatcgctatataagaattaaaaatacatgttattgatttacatattatgttagctgctaaaatgtctagtatatagtttccattataattttaagttctgacaccattttcctactgaagcccccaaaatttggtaattaagaaatgtaacatactttttaacaaatttgaaaactgtcttgtcaaaatggtacctgatatatcattactacataagaattgagacttaaaacatttaaagaactgattcacaaattttaacaaaagtacttagaaaattagacctttctttagtaccctgtgcttctcttggttcttgttatttctgtgttatacagaaattccatatttaacacacacacatgcatgcatccgcacacacgggcatcccacacacatagaagtaaaattctaaccaccccctacctctatttaatagcttatcagctatgttcatgctacaactgcttcattgaagggaagtcctattctgctgttttcatattagctttgttataatgcagtatttttcagctgctcatgctcattaccatacagctggtatgtagagaagagaaggaaacaggcagaagttgtactgtctacaagaataagaagctctcttgcagatggggtgctggcagatggggcatgtgtttgaacaggttttactaataacagagtgcgttaaaaatcattctgagaaagtcctaaggtcagtgccatggattcagagcttgagaggttgttcagctttttcgtagacctttttagaatattaatcgactcttacaaaaaaaaagtaataaatggcaaaccatacctcctccaaaaaaaacttaccagtttttttcttctttatattagtatgttttttctttagtataattttaaaagcattaatactgtcacctagtatcatcaacttgtgttgtcgtagtcttaaatatatttacttaaaatgtctacatctatatacaaaaatgtagatgctttgtgaaaatggtaataacattcttcagtttctgtatattagaatatttattagaaagtgaaaactaaagatttagatatcagtgtgtatagtgttgactacgttgaagattaagtcgaggaatgttttatctgcatacaaagttagaagtgaatggtatgcccagaaatgtatctattgttaaaaatatttcttgcatattttgcatattcacaaaaccaaagtgaatgaagtataattaaaataaccatcatttttattagagtattttagtgggatcttagtctttttggcccttttttattaactagtatttcttgatatatcctctctcaatgggagatgctgatgaaaagcaactgaagcaaaataaagctaggcaattttgtgtaacatatgtagtattcaatttaattttaaaggaaatagaacttgttcttttcatagagattaaaatatgtgaatatatataatatatatgaaatgtatacaagtatagctagtttttttttatactttaagttctagggtacatgtgcacaacgtgcaggtttgttacatatgtatacatgtgccatgttggtttgctgtacccattaacttgtcatttacattagatatttctcctaatgctatccttgccccatccctcaaccccacgacaggccccagtgtgtgatgttcccagccctgtgtccaaatgttctcattgttcaattcccacctatgagtgagaacatgcggtgtttggttttctgtccttgtgatagtttgctcagagtgatggtttccatcttcatccatgtccctacaaaggacatgaactcatccttttttatggctgcatagtattccatgatgtatatttgccacattttcttaatccagtctatcattgatggacatttgggttggttgcaagtctttgctattgtgaatagtgccacaataaacatacgtgtgcatgtgtctttattgtagcatgatttataatcctttgggtatatacccagtaatgggattgctgggtcaaatagtatctagatccttgaagaatcgccacactgtcttccacaatggttgaactagtttatagtcccaccaacagtgtaaaagagttcctatttcttctccacatcgtctccagcacctgttgtttcctgactttttaatgatcgccattctaactggtgtgagatggtatctcattgtggttttgatttacatttagtttttaaagctagtataagttacaacacttttggcaactcagaaactcatgcttcagatttcttgtcagtaaaatggaggtacaaaatactgtctgtgaagttaataactatgaataaataaaatgatttaaatgagacctgtgagaagtggtgtactttaacaagcattgctaaaactgtgtattccttttaatagtgaaagtttagattctgttgggcattttaaaggtacagaagtcctaaactgtgaagttgcatttttattgctcatcaaacttattttcttccaatttaatttgtatttggactgtatgactgataaatatgtagaaatgcttttatgacttttccctgagggggaaatatattaagtattataaacaatagacagtataattataaaatgataaacctgtaaaatgtatttttcacctacatcatcatttcttatttagaaaattatgagtttttaaaattgtgttcaaaattaatggttttctgttgttatttctgtagctctaccattgttgattagggtatgtcttgtgtggctaattatctcatataaatattttttattggtaccctactatatgcaggaactatgtgaggagctgagatacaacaagaatcaaaacagaataaaaatactgccctcatggaacttacagtctattggagaagacagacaaatatttaatgtgctcacactgtaacaaggtgttggcttcaagcactgggaaatcagagatagcctgtatacagataattcacatattttcatctccaacccagacctttcttctgggccagaaccagttggagcatgagatttggatacccagttatctccaagatatctcaagatcctcctcacccctggcccccagaaaacaaaaaacaaccaactaacccagtccttgttatccaattgcacaagtgagaaaacaggtgttatctttgataccttcctcttttttacttgccatgcatgacccatctgttactatcctatcagtttgatgtcctaaatgtctcttaactgcccactcctatgcatctctgtttctacctagctaatcgaagccatcattatctcttgcctggtctagtggaataacttcctcacacagtctctccacatctactctcaatcactgccattctattttcagcattacactttgaatgatttttttcaagaaataacatctgattatatctttttcttaactactatgcagcattagcttttcattactcttatggtaaaacccaaaatcctttgaggtcttgggtaatgtatcccttgtctacttcttcagcctcatcttacacctgccatcctacctctgttcagtgtcctctagctataggtggttatccctccctgctccgaacctagctcagtcattcattcagcgagaagatgccttctgtgaacccacagactaggtggaaacctcaccattcttaacactcttcgaacactgcagcatttaccacagttgcagttttgctttgttttgtctcattatcttgcccactatgctgtgaactccagccatgaaacagacacaagacttagtttagcttatgttaggcaactaagtaactattatattgaatgaatgggagtatctgtggcagactgaaataaccatgtaaatctgcatgtgttgtaattatgtatattaggtagaatataattatggagatacttgagacaaagtgtagggtagaatcacagagatacatatgtttcaatacagatgttcactgttaggaatgataaccatacatggatcaaatggacaattaatgaagatggattggctctttaaaaaaggccttatgtaatgaagttaagaaacagggcccaaatgatagccacatattgaaaagcatgtgctctatgtattcagatggaaaatcctgtgtatttgttactacttggttagtaagtcaagtatttttaggctgtgtactaaaaaaattttcaatgaagaagtcatagtcccttattaagtgtatttgggtttgagtaatcaacaaagaacatttgaagcagtttctttcataaatattatgattcataacttttgccagttttccatagggttcaaatcattttggtaaggaggaaaaaacctagtagcttcgttgaaaggacactgcaatggagatcctttgatgtttgtgttttcagctctcttttatttcatattttagggaaataaagcaggacatgaaggaaggagagtaagacatagaagtcctgagagggtttgggaaaaggaagcaaaacccatttgttgttgtgtttcttccagtatgcccactgatgactgcctggcctcaatataatgtcacagattataagagcagttcttaaccaagcggagccactggcagtagtgtattaacttgttgtcaagctgctcctggtgaattcccagaaataaagtgtatcacagtacacaattagctgcatagtccgtctcttctttctgcaagtctgcagtggctctagtgtggatttcatattcacatcacacatttgcatactgaaataactaagaaaacatcttagagatatttaaactgccaatatttcttgctttgtatatagagaaccctatttttatttttttgtaaactgaagaccaggacttccttaggttcaaatcccatctaccttaagtgaccttggggtcgcttaaaactctctgtgcctctttcctcatctgtaaaatagagataataatagtactttctgcagaaagttgttaaaccgtttcattgaattaatataagaaaaacacctaggaagagtgcctgacatatagtgagtttccttccctgtatacattgtattcttaagaaaacatctttttgaaaaggagttgcttattcaaactggaataaagtttgcagcatctttgatagtgggcactgaaagagcattcaggtcagtttaaaatctagtgtttatttttcttgtatacatttggtttaaaatattgccatgtgcatttcttgaaaatattatctcttcctctttctagttgtatatattgtcatacaaattaagcagtttcacttattagtcaaatggatttgtcattttttattgacttgcctgtccttccctcctgagttgggagtgcttccaggatagatattacatcttgctcaattttgcatcttttttgcttagtgcttggcttttagtaaccctggacaaatatttctaaaatgtggtagatctagatctaaaaatataacatttgaatcactgcttaattttttctccctcttgttttattgtttagaaaatggagaatcattatatgactcaggattcatttggatgagactgataatattataatggaatttggagttagaggctcacatgaattgctctcagtcactagctttttctgtgctacctataaatgataaacaaatattgtgaatctcacaaagttaaagtctttgaaataaatggattggtagggggtaaattacatcttagtctaacaaaatctgtttattgcaatggtttaataaaagtcaggtgactaaaaatatcttggaaaataccacttaattttaatctttgctgtttatgttgtgatcatgatgattaacatatcttcatttatcttctcttgaaaactattccaagttcttgttgtttttttttttttttttttttgagagagagagtttcactctgttgcccaggctgcagtacagtggtgcaatcttggctcactgcatcctctgcctcccgggtttgagcaattctctgcctcagcctcccaagtagctgagattacaggtgcccaccaccatgcctggctaatttgttttatattttcagtggagacggggtttcaccatcttggccaggtgatggcctggtgaacttgatcttgaactcgtgaccttatgatccacctgcctcagcctcccaaagtgctgggattacaggcatgagccactgcgcccggccctaagttcttttttaatatgctaagttgtctgttgttggaatgactttaaaggaaaggtttaaaatgaagaaaggaatatgtgtgtgtcttctttatcatctcatgaactaaaaaatactggaattttaagattacactaaagttcagcatccttttgctagctttgaattgatattcttttgggtagatttagactattcctcataggattttttcatattgtataattatgaacatacattgactcataaacttagattttaagaaatctaagatttagctttaaaaagtggtctcaatttaccatgtagcagttcgtaacagaccacagctatatgcacttgttcatatcaggagcagttcttaattttagttataattcatatttgtttaatagtcatcattccatcattacataccatgccttcatgcaactggtgggaaacttatgatattcattccacataaaggagtttttagaatcatgtattaaatgtgataagctttcttttttcttttcttttttctgttctgttctgttcttttttttttttttttttttgagatggagtctagctctgtctcccaggctggagtgcagtgttgcaatctcggctcactgcaccctccacctcccgagttcaagcaattctcctgccttagcctcccaagtagctgggattacaggtgcacaccaccatacccggctaatttttgtatttttagtagagtcggggtttcactcttggccaggctggtctccacctcaagtgatccaccagccttggtctcccaaaatgctgggattacaggtgtgagccaccatgcctgtcaaaagttagttttcaaacatttggaggtctcaagtgtttataggtaaatattttttaaatcatgtgctaaaataaattttaagaaagtgaatctgtaaataaaatatttctatgagaatgttgagaagatagcaaagtgcttctaactatcagtcttacagttcactgcacataaaagacaaatatacattcagataccttttcaagttaagttgtcttgaaaatatttattgaccttcagagtcttttggaatctcctaaacaggtagggaatcatgtaagtatttgctagtcatagaattcttgatttttttttaaagcaaataatgtaatttgttaaagtatatatatgtatataggaagtactttaaaatttaaattaatgttttaaaaatgaatagaatcctctttttaaaatgtagtgggggcctgattactatgttcttccctactcacttgacagctatgtcaaacacttgaatacttaaagaacaagcaagctacataaaaacaaagaattgattaaagatcaccatgaggcacactttctagagcaagaatccagtgttatttttgtttctcactggcctagcaccaaaacaaaataggtactcaataaatatttttgaattagtgaaatcagtttgaattcacagactttgattcataactagagttttaaaagattggttcaagttatgccttgtcattttgaattcttctggaaaattagtaagatgcttgatgagaagagatgcctatattttaaaaagtgcagtaagtatgaatctggactttctgtatctgccagccaaatgttggctggcattttactatattttatatatatatatatatttaatatattttaatatatttttctaggcttttctctatgcatacacagtgcgcacacacataaaatatatgcttgttttttcaaaaggaccgtaccttatattgtgttatcgaatctcacataccttctagttttacagtcacccttccatagtcacctagctctgtacctgatgaacggtttctgagacttcaaaatgctctatagtgaagtctgttctttagaaaagaggctttgagtctgggggccagacagtagaggtgtggggagaaagggctggggtagaacaagatgaaaattttcttagtgagctatacctttttgaataggacaggttctttcagtcacatgttggcaagaatcggtgaattgtgtactcacctgttgccccgagtaacttttcggattgttttttcattaactttttctgggaatatacaatttcaatgaatgttttatacaccatttccctgcgctgtgtattcaggagtacaacttgcttgtatgtatgatttctagtgtgaatgcctgttcaaacagaaagtccccttgtttgtcagtgtttgtaactaaaataaaaagtcaacagtcttcacacccacacagtaaagttctcctagtacagaaagacacataatgccctaagatggaatgatgagtcatgacaggaggtattttgaagaaatttaacaatatgttaaataacattttaactctagtattaaatgtttcagttaggagaaataattgttatcacatgggttatcccctaatgaaccttgtaagaccagtgattagaagtctaatagatgctgttaatcctgcaagttaataatagtcgtatttccatttacatctcaggtccacaggtggtaatttccttcttccctctcaacacttatcaaacttctgttttttaaacacagaagcaagaccatgtcatttatgaactcccagactattaaaataacttctccattttctgactcactccacccaatttggcaattgatatatagtccagtattgctttctatttgtttcttgtctttgtgggttttttttgttttttgagactgagttttgctctttttgcccaggctggagtgcaacggcacgatctcagctcactgcaacctctgccttccgggttcaagtgattctcttgcctcacctgtcttcgtggtttttgttgttgttttgttttgttttgttttgtttttggtagtttttgtttgtttgtttttgagacagagtcttgccttatcacccaggctggagtagagtgctgtgatcatagctaactgcaacctcaaacgatcctcctgccccagcctctcaagtagctagggctacaggtgcacactaccatgcccaggtaattttttttcattatttatagaaatggggtcttgctatattgcccaggctggtcttgaactcgtagcctcaaatgatcctcctgcctcggcctcccaaagctctgggattatgggtgtgagccaccacacccagcctcatttcttatcttgtcacctggattttaagtatcttgtgggaggaaggtaattgcaatgtttttgtttgcccttggtagcatttcgcagatattgggacataggaggtgacttatttctcaattgattgtttttttaacctaaaattttctcagtttctgtcacctttcccccatcaccaggcctttgcctcagatttcccagatgtagtagaagcatagttaatatggacctaaaataacgattcaataaactgaataatcaagatttacttgtaatttacatacatactttttgcaaaatataacacttttttcttctttaacaatgtctcgtgttttagcttaacttgagctaccacatatctaatggaaatcagtaagaattattgatcttgggaccagagaccaaggttctaaatatagctagctatttaagcatgggcaagacacttaattctgtacctacttttcccacctgttaaatacagctaccatttctagggtatctgttatgtgctaggcattttctgtttcctcacagtgactctgtagtttaggtggtaaggtaggtgtatatcattactgttttataaaaagagaaaacaaactcgtaaaggtcaagtaaccacctaacattacactctggtaatggcaaaacttgcgcttaaatccaggtttttctggttccattgcttgtgttcattccacaaacaaagaggaattagatgacatccaaaatcactctatggcttcctgctgttaatattttttgctgatttagtagctataccttttttttttttttagtctttagctctcttctcaaaacttttttatcatgactaatcactaatttaggaaattaatcccagaattttaaacagttcttgaaatgaatacaagattaatatatcacacctatgggaaattcttctcaagtcaggcaaatatatttgaaaatcacaaacagcagcaataaatatggatttgtgtaaagaaagcagaagagtgaatagggtttaggccttatattgagggaaatttgatttcattctttctccatgttaaattctttcctgggtcatatttgtggtttcatttttcttgaagtcaaacttacattttatactttttttccatgatatctgtcttactttactgaaggtattactataagaaattcacaataaaattttgagaggaaacttatgaattccaaatgcttattggtagtttattttggattttaagcatgaaagattcattgctttctggataaatcagtaaactccagggtgacttgctaactttgttgtcaaattattgcactgatgccggctgtctgtgaaaaagtagaggatgttgacttagtattagtctgacagctctgtaacaatataaattttagaggagcgaataaaacccccatattattttattctgtggctggcataaatagaactcttaacttgcactattgggtatgaattatgtatgtgtgtatatttatatatttgtagataaatacatttgtgcatgtgtccatcagatgactaatttggcccaatctgaaggaatgaaaaaacttacactgttggaactaatactgcagttgattagtgtgtcacactaatcagaggctgtgtctttgtaaggagagttttaaatagcttaaacttcaagaattaaaacttctggccaggcacggtggcttacccctgtaatcccagcaccttgggaggctgaggcgggcggattacttgaggccaggagttcgagactagcctggccaacatggtgaaaccccatctctactaaaaatagaaaaattagtggggtgtggtggcacacgactgtaatcccagctacttgggaggctgagtcatgagaacctcttgaacccaggaagtggaggttgcaatgagccgagattgcgccactgcactctagcatgggcaacagagtatgactatgtctttaaaaaaaaaaaagaaaaaactactacttctgtaattatattaaaatttatttaaaagcagtcagctataacattttgatttgcagcttgatttacaaaggtttggattatatatattataaacatccccttccccccaaaaaataagtttaagtaaagtaagattatctattacagtgtttagtgtttgggatttttttctaaggcactagtgtcacagtatttgtttacttagatggtagttttgaactaaagctggctcatcttgttttattttacatgttctcaaaaattttttagaactaatttcagtatgagttctaacagtcagatgtttgtgtatattacgttacactatgctcttgtgttaattgactgtatttccaatggagaaagaacaaagtttcaattgatgtatataatattctgtgcccaacacaaagtaggtgctaaacaaatgcttactgaatgaataaatatggaaaattgcttattggccaatactgccacctatttgtgttagtgaaaatgtaaatgctgggcatatatactattccagttgatatctgtaatagaacaggtttattagtttgccagttttaataagcaaaccttttaataagcactcacatcccagaaagttaatgacagtgagttagtgaaatttcaaaaagtatttcagtctcaacatgcttggcattcctttcattgatgttgaaattttgaactgtctgtaattttgaactgtctgtaatctaaattctgtgctcagggcaagacagctaactgtggtggtttcctagtcctttctagagtacaaagtgttgtcatgataataataaaatcactgacataggaatataaacacattctcctactattataaaatggaagtgtatttttaaatgttagcaaggtaaagaaaagaactaatagtattcatagtattttccactaattataaactttttagttgagttaaactttttagtactgattgtaatatatgtttaagctttagaaaaataatgctgtggtttctgatatttgcttgtatggccaattcaaaaatattaattatttgttttgtatcattttctggatgccctagaaataaatttgagtaagatgtgaatctttagggaggagataggggagaaagatgtataaaaataatgacaatgaactatgataactgcagtaatagaactatatataagacggaaataatgaactcttgagagtcaaggtgaggtaaagtactctattaactacatcctttatgttgacattatatgtaaagataagtatttctctgaagatacatgattcgcttaagaccatttgtgtatatttttctcactgtatatacactattttttctttctgctcacaaccacaacttgcccttctaaaaacttaagtcgtcccaattatttgttcagagctctggtaggagctcacgggttaatgattctatggcatcattccccagaggcaaacatatcatcatatatttgtgttgtttttttttcctcgttacttctaacttctatgctagcacactagttccacttctacacctagcacagtggagttgtgatgagatgagcattgggtctgagttagcagatggattttattccaggaccttattgttggtcattccctttgctaatggaatttcaataatgcttctaggttgtcagtcaacacaagaattaattttttatagaatgggagaaatgagtgcctagctgaaattccatgaaaattattgtaatgaaaatatatttaatatagtgaaatagtaattctaaaaatatagatgacaaacattttcttagctgtttggttgactaacttagctatttggtttcttagctatttggttgaccttctttcctgtgaacttactacttaatattttagtgtaaggcataaaatactttaaaaaaacttatttaacatagataatagactgatctaaagtttaaaatgtttcttgtcacagagataagatagataaatacagacacaaacccataaaaatttttaaatattatattaccaaaaacttaaatctgaataaagtttcaaactatattttagccgagtggtataaatagaaatgcaagtgattttatttgctttgttcatattaagattatgatgacagtaatataatagatcacagttttcattgatagtacgtcagagaatgttgaaaatcatttttattctgttaatttgaaattcagttagaagcttagttgactagttttgcttgacagattcttcacagttcaaattaacatcataagatattagggagaattgtttaaatacttgggaagacaaggtcttcattagtacttataaacattcgcatttctcacttaaaggataaacaataaaaatcattcccaaatgggaaccatggtagatggaggatatgtggtagtagaaagacttctcagtgtagaattttttgtatatcattttgatgtttgaagcatacaaacctggtaataaaacatggatctacaggacagtacccacatacaaacaatatttgtagggaacaggttttcctgtaactcacagccctaagtaggagccatataaccatgggagcagtcctttgaatatacaaagttaagagatattttctttaggttttgagacagtatcccctacttcattaaagttggtctcttcttaactgagtagatttaatttgaaaaggttagttcaactagaaataggacatgatttcagttgaagccaaagtatatactattatagcactagtattacacgtgtcttatggaaaacttgttaccactgacaaattctggatgaaatttcaggtgttctcatagcccagaacattgaggaaaatcaccttttttccccctttcctaaatcttgagtcctcagaacttctgacaaaagagtttatgggtccattcctaacaattagatttttttgtttttttgcttcccttcgaagtgagattatttagataactaacttctgctgagtacatatacatattgttcctgagggtaagaattaatttaagagccaggtaggagattttttttaaagataagtagacagtagaatataatttctagaagactcaagatgtacatcttcataaaattttacgttatcttaatttcatttatgttttaaagtaaaagatgttgcattgacataatgtcttagttataaaggtaaacatttttatttttaaaattatgatactactttatgcagtatttagaaccattttactaaagaaagccatttaccaccatagcagagagtattaaaatgttagtaagtccaaatggttgttttgttttgtttttctggagaaacttacttcgaatagtggtaactataggcacacaatttacttgtaataaagaacactaatggggagggtaggagtaaattaactagaaccttaataatgggcttttctagctcaaggaatcttataattgtccctgaaggtagttgtgaaggaagttgcaagatgaaatatctagatttgtgtagtagtcattccaggaaaaatgttgccttctggcaaactttggcatctttattatgtaaaatgcctttttaatgataagcaatatagattgtcaaaataattttggagatagaagtctagtgcctcctgtttactccagattaaattagcaaataaaagtaagcctctccagccagcacagtgtttgctcttttggggataaaatagccttttggccaaactttacacacttaggtagtactagtttgttaatcttacaaagatattcttatgattagtaagaattgatgaagtgtatggcaatttccctgaaaattactatgttttaaaggattgaaatggaaaagcataggcaatgtttttattaaatcaataccaaaatacgcagtttgaaatataacttagattacataatttctgcaaatgctgtcggctccaaaccagtttactttattgtcagtcaaccccagagtatattaatttctcttttcgggattgcctgttttgtaccatccaagctacttgaaaaatctgattagtctgtgccaggactacggtaggatgacagtgaaaatagcatgctttgcctcagttcagggggaccgtatgaaaatatatcctattttcaaaatgtttctgaaaagacttatttgtggaaatgttccttgctatttaaaactcttcaaaaatgcaggttttacaaaatacaaaaataacctcttgaaagtttgaaaggggagagggggctgtcgtgtgggggcggtctctgctgcccacatcctccctctgttcagtgcgttgtgttcttacgggttccctggagcggatcaccatataattgatgtgcagtctgcattgctgaatcctggactgcaccattctgtgttcaagggaagatgtaatctgatccctctgctgctgagggaggaatctgttcagtcaaggctttgacagggcatagtctcctccaataatcttctccgttctctctcattattccctcgagttcttctcagtcaagctgcatgtatgtatgtgtgtcccgagaagcggtttgatactgagctgcatttgcctttactgtggagttttgttgccggttctgctccctaatcttccttttctgacgtgcctgagcatgtccacattagaatctgtgacatacctacctgaaaaaggtttatattgtcagagactgccaagcagccggacacacgggggcacagaatcactgaaggggaaaaatacagaaaatatgggtttctacggcacattaaaaatgattttttacaaagtaagtaagctgttttcttcctatgctatgtgattgtgtgtgtatgcctgcctgagaaggaaaatctgtatcatcagagatggctgcagtatcctctaattttggtgggtggctggatgtctggtattctgttttatatcatgaatgtaagcaaataacggaaagactgtgagctggaaaaagctttaaatttaagataaaatgtatattttaatctgagaatttttcactaaaaatacattgagactttagaaatgtggctctgttaaatgctgacctggaaatactctatccttagttggcttttagtgaaccttttgccttcaatttttctataccttgtatacagtttttcttttttcattagttgactctttaaaagtatagaatcatgagactatttgggatttatatttaaagagatcatgtttactttctgtacccctcaaaaattgcagaattaatttttggttctgaagtcagtattttcagtaaacagtacagtatttaaaagacttctatagtcatacagaaggtgctttcctttataaaatatttttaaaataaaatcctttctcagtggtttccttgatacacattttggggcatgtttattcattgaaggatttctaagatggaaataaagtttgcttttagtggcaaatggtatttgcaccaatgttttttacaaggtatcctgggaagttctgaaagccagtattacaaacttaattagaataaatatcaaagtttctcctaattatgaagataagaatttgtggaagttatcaaaaaactagattttaacatagtctgtactgactgaccttcctgatggattcatttgcatgtatgtttatatatatatgtgtgtatctataaattatatacacggaaacacgtggctacctatatacttcagcttttatatttatatttgctttttaataaaattatttggagaaagaaaataatggtttctgagaggaatcactttaaaacaatgtaatgagtatataaacttctaaagggaagaaaccagccagatcaattccatttctttccagtaggtatggcatgaactaaggtataaattgtaggtgagcttgttgtaccctcacctctagcccctaatgtcgtaagatcatcctagtattttagaaaactcttcgtgatgacaaaaacatggtctttattattcagtgtggctctaaaacattttaagtctctttagtatttatattgtatcatgctatgttatgatgttcccatgtaaggcaagaggtaggaaaataaggctgctgcaaagcaaggatttttacctacttataaacatgaggatagctctctatcactttggagtgtttttcctcctataacttgcagtaatacagtgattaattagtggacacaaatcttaaaaaggggttgttgaaatggaaaaagtcaaatcttaccagattttattttctagttttaaaatatacatatagtcaaatgagaatatttcaatattgatgttgacaagaaacaattctgaatatatttatagtataacatggcataatgaaacaccttaaaatgtagattttggatacaccttatagaagaggcataactgattcttttaaaacctttctgttgttgcttttcaaattagaatattttgtcctgagtatctgtcaatagagctatattctgtatatatatcaattgacaactttcccctcagcgttattatactatctccagatttctcttattattgatttactttttaaaactgtccttattgcccatattaattgctttatgccttatcactattcaagattatttatactgctctttaccctcttataatggtatgagtgtttaaaattgagtttgttactttcataattatttctgattttacaattttgaattttatctttattaaaaacatgtttttaaagaaaaatgattcacgaggaaggcacgttaaatatgattattttctgtgtggattaacttgagatttttccctatgttggttattaaattggaaaacatgaaagatatattatctcaaggaatatatatatataatgaatatcagttttcaaatcatcacacactgttcttctggataaaatattaggggggaaaattcacctttccattccattcagagtaaaacaaagtagaacaaatagaagaaactatgaaacatttattttatatatttaattaacattgtaggcatatgaaaatttaagaatttaaaatattaaccatgctgactcaagatttgatagttagacgatttttaaaaaacatatcttgtttgttttaccttatagatgaaaagaaagttggaccatggttctgaggtccgctctttttctttgggaaagaaaccatgcaaagtctcagaatatacaaggtaatattaaatataatgattatctcatgattctcagttttttagagttgattctgaaagtgttttagtttattcttgtagaataataatgttactagtacggttgactgtttaacacattttgagattaccggaaattgagaactatatcattttaaataagaggaatgccaccttagtttatgttttgttactaatacattttgaagtaggacacaagcagaaattatcttgtgaacaaaaatttaaaattacaatttttaaggaattctaaaaagtaaaccataaaatatgaatatgagcccctgtgtcttgttactataggaacagctgttctagggtagacatttatgtatggaaatttgaagatacatttattagcagtgaagtaaaacaattactaactcaatcttatttcatgaaattaatgtgttagaaattttcttttatgggcttggtaggtcttaagtttttaacacatacatttcttttctataccagtagaatagtttccataacaatgaaacataattgtaatctttttctacttcagtccccattatttttggattttacatcagtataaaacttttcccactctcacagttattaggctaacttaagcacaatgtctattgcccagccaattaaaagtggctttgaatgtcacttggaatgtcgtattttaaaatttaaatgacagaagtatcttagagcttttttttttggtctttaaaacagaattttttgtataattgccagtttttaaaataagtgattgaaagttaacagtagaaaccataagaacttcatggttatgacatttcttcctttctctcaaaatatttcaatttagttatttttattctataagatgcactaaaattaacattgctcagatataatttttttattgaacattgttgcagattgtgcctcttacagagaaatagcttgtttttatttcagttgtaaaaaatatttctttcaaatgctgctttatgaaatttgaaaatcatcataattggaatagtcttacattcaaaaagtgaagagataacacagaaaagaaaaataaagctatctcttattcaggagctgcttttcggtgtgatttttcttatgttaactgtcccccttcctgttactctttgccttttgacagttttactgtgttttggcacttccaccagagggtgtggagggatagagagacaagtgaaactattcacttcattttgcttttcccttctagtcatgataaagtatttcttggatgagaaatttgcatttaatgttaaactaactgttaatgctattttttaaatgtatcagtcttctattaatatggctgataccaggacaaaggtattacattgttcacttgtttttaaggcagcttagaataaatgagtgctgaatttttattacagaacacattttagtaaaatttgtctttagttgctcactttttatattgacaaagcaactgaaaaggcagaagttttatttttagcctgttaatatataattttattaaagcaaaattagaccaaacagcatatttatttccatgtttttctggagactctcggttaggaaggtatatatttggtattttgtgatatttttgacagatttgctaataagataaaactatgatttttccatcaaaaatttgttataaacatatcctattttattttggaggaaaagaaaatagaacttacctttgaatttgaaagaagaaaagatagatttaattgctgaatgttaaagattgtcttcgatttatactattagaggatttaaagggagtagataagaagacttttctgtaaaatctggtgactgagatacattttttaacaaaataatgctggatagaaaaaaacagctggaggagttgtttcattagtaatttatcagagtttgattgttaactattacgatgcaatttgtaagtgttctttgaatttaaaataattaatcaacatagctcaatgaattgagtgagaatttcttttcaaatataattttgttaaaccagaccttcctgctgctttttttccaaggtctttaaaatggatacccaatatttaccttgagtttagtttaattacatgcagaaattagaaaggaaagtttaatacgttagtaatatagttatatattctttctgctattaattggtcatttttagggtagttagttttagagagatcatctagtcgtcctcattttactagtggcagaattgaatttcaagtttgtttgagacagttaaatctcaaaatttaaattattgtataaaaatgtttagatgatcttatttagttttgcatataagttcttaatacttacctgttttgaataaatgtatttctaatcaagactgagtcctgtcaactattcagaccaaactttataaatagcagtaaaaaattagaaatattatagacaattttgaaggcatctgaggcaaacttaaggaataatgtgattcttgaaattcaaaacatttgaacatagtatttctatgtctgagttttttaaagctttaataaagtatcacaaccctttttataaagaaaatttaaactttacttatgtcaccaaagaccagtcataggagaagtgggacttgtatgttctaaggaaatattttaactttatttgtatatattaacaacttgcagttttgtcatggagattctgttatttggttgcaattagcatttctgggataataacaggcgttatataggagggaggaaacttactgtaagaatagcaaataggttttgtgtacctgtgatctctgggaagaaaggtaattaattgatgtttgaaatatgccagatcatcattctttgtactagaatctattattaataagtatctatctttgctttcacttttgtttttttccttttatcctttctctctctcttttttttttttttgtagtaccactgggcttgtaccatgttcagcaacaccaacaacttttggggacctcagagcagccaatggccaagggcaacaacgacgccgaattacatctgtccagccacctacaggcctccaggaatggctaaaaatgtttcaggtgacatatctaaacatagtacttgaaatgcctcacttaattgctgcaaaataagggattattcatccgtaaaaaaagaaggaaacactgttacatagtatactgtggatgaattttgaaatcattctgccaagtgaaagaagccagacacaaaaggccacatgttgattctctttatatgaaatgtccagaataggcaaatccatagagacagaaagtagattaatggttgcctgacaatgaggggaggagaaggactaactgccagtaggtacagattgtccttttggggtgatggaagtgttctggaattggatagtagcgatggttgcacagcaaggtgaatatactgggaagcactgaagtatatacttgaaaatggtgaatgttatgtgctgtttatttcaatttaaaaatgctattaaaaacaagggaactcaatttttgaatgtcataattgagaaagtccaatacttatttgtgtaactgtgcactgacattttttttattgctttttaaataataagaccttccttcatcttctcctttgtttgaatttctgggttgtctagttttctaaactgcattgcttgtaagttaggcatttaagagtaatttaagaaccattctagctagagttaatacagcttcttttcttcagtgccatgcttgtatatggcttaccatgcttgaatcctgattactatattttaatagtatttatattctttggatgtgtacagggaagattggttacaaccaaaccctgaatattatgaactttataacctaagatagtgatattacattgatttatctcttcttccagtaagataatgcatatacatcaagtgtttagcaaaatacatggcaaggattaagtacttactcaattatttgttgcaaataataaataatataaattatgtaaagtaaaaaatatctgatattagcctccatagtaatgtaaaaagaatatactaccctacctctggctgcttgaatggttaggtatttaatcaatgcataaggcaataatcgttggtaaagatgatactttacttgacttttagtgtttcagatttttaaggtgttcactttgtaaaccctaatattttaaatacctagatgaatatgagataaatgtgtctatttttttcttctgtaggtagaaaactaacttggagcagtattgcttttaaatggttttttaaaattgttttctacccttcaggtgatttctgcaaaactaggataacctttttttggttggtaactgaaagatttaagccttgctctcaacagggattagtttttccccgcgttgagacaaaagtgtctcaagacatttttcatttttataactgaggggatggggcgcaggacactgctggcatctagtaagtagaggcaatacaaaggacgatctccatgccaaagaattatccagcccaaaatgtcaatggtactaaagaatctgggttagaggaatgaaagttagattttattgttattaaaatgttataagccactgtaattacaaagaattttttttaagttttcattttcatagtcatttgttgattaaacttaccttttccccagggattattcctacccttcctcctgagttattttgcctctggttcatgatggtttgtaaaatgttaattaatttttattttttcctatgcacaccctcagaattctaaatttttaattttgtcccatccttttaaaaggaaatttatccgtcaataagaggcgaaaataaaactcggacacttgtatagccttaaaaaaataatttaaagatttttatcatctctgattaagccaaagaggtaggaatatggttgtatgttgccatgctaacacatgcatgtagtgtgaacagcatatatcattctgatttcatatcacatgatttcactgatttcatatcatttttataagaaaataccaacatgattgaaaattacactattagcaatagttttatcatcattgtagataacctccaaaatgcttattttatatgaaaaggcttatttcttttcagaaggtagttcagagtaagatttactttttctgtggtagggctcattttttccaacgttttggaatacctgaatattctttgcttgctatctaggatatttctcaattttaaagggactctgtagtattgatctataggaagaggcatttcagctttattttagtcaggatattttagagagttcattgtacaccgtttgcttgtttagctagcttatgctttttaaataaaatgtccaaatgttaccgcatgctgtatttcatcaaatctgagacaccatagtttttttagatgcattccaatttcggaaatgttaatgtgaaaaaaaaaaaaactgtattcaataagctaaagaatgttagaatagcttttggtcctgtaaaaggagtaggtgcagtgaagaaaacatgttcttagttaacaggaatagcatgtggaattacccctgcaggtatagtattatatacatttttttcagataaatattctgtagcaaagaaatctgtagcaacaaaaggtcaactggacaggtagatcaggaccttcatcttttgagtaccatgtctgtctctgacacacccaaggcctcattgttgtacactgccctttattttcagggctggtcccagagtaactagaaatgattcacttgcctaaaggcttactgtgctttaatgtctaacttgtagagatataatttcataataaagtggggagtcatgagttgacctatgtgagagagttgagggataattaaactggcatgttttctacaaatatactgtgggggcatttcatgatgacttttctgttgcataataagcagtatgggagagcttgtccccttactgttctgctattgctcctaaggcagtgatacagcattgctttggtccagttccatctattctgacaaagtactctgtattctgtttataaattgagtatgaacaaatgatattccattttgcataaaagaggactgaaacagaatgccactagaaaatagaagtgcaaattaaagacaaaaaccaaaaaaaaaaatagaaagtttataccatttgaattttcagtagccattgatccactttgtatggcagtcagattgatgcttcagttaatttagggaagcgagttggtcatttttttggcttgtttatatcattcaggtgctaataaatggtttttcacttaaacttttcagactgggtaaagacattaattacatgaacattatcttatattgaaatttgtattcagaagcatcttgagaatagcacaaatgacatagaaggacaagttcttagttcaaattttgacattgtctcttaaaaacaataatttcattaatatataaccattcttgcctttattttcacgctctataaaacacataacattaaaaagttgtcaaaggggaaaaaacatgtacatctgcttaaagttatacttggaaatatattggagactcaataaatgttagtttcttcccttggtcaagaaataatcttagtggacctcagaaattttctcagatctaaaaagggaccgttaacactagcttcacagtattgctatgaggatcaaagataaagtgatatggaagaactttaaccttacacagatgccattagctttacattattttaattaaagcatattggaattaagctatttatgtactttgaaaagatgttatactaaaaatatttttaaatgataagtataaaatacatgtgttttgatattaaaaatggagaaaaaacttctccattggaagatttaattaggtttcatgggaacacttagaaattgtacatgttctcaaatctactttatttacatcttatttgctaatcaacaaaagattaaaacctacaggatagtgccaaaattgaatttattattactttgaaattgtatttgggacacagtgatttaaaaaccttgttttgtgtgaaagacttgaagaaagtcatgtatttagctagagaagaccaggagaaaatgtgatgtctgtttttaacattttaacgacttttgtataaaagtaggaaaagattcattcttggtttcttcagaaagtaaaacctgaattatgttctttgagtcagattttggcattaagttaaacttagcaattagaactggtccacagtagaatgggagataataagttactcgaatcctggaactgttcaaacagactggataaccatgtgctagaggtgctagagaagtgagttctttaccacatgaaattcaggtgcagtactacccaaggtcataaaacagtctatctaattgagacactaaagatcatccagaccagtggttcccaaatgctgctttgtgagtcatctacatcaacactggatgagttgcttgtttaaaaaataaaatgtcttcagccactgaatcagactatctgctggcgggttttagaactcagtaattcttgctgtccttgatcggcatttgggaaccactggcctggaccaagcctctcactgtgtaggtgggttctctgagactcagactcattaagagacttccatgttgttctacagtgagtttcctggtgaagcccaggctcttgattgcagtttagtgctccttctactcttctctactacctctgtgccctaatttacctgaaataaaacaccacttccatggggaattttcaaatttgcatttgcaattttgtccttaaattaaaatttgcttctgaacctggaatgtttttcctctccctttcaatgtgttccaacatggctaccatgaaaggccaaaggaagtactttttttttttctcttttctgagacaaccacttttcaagtagcagtaaaataataattaaaagaattatccaacactctgcttttgcagataaactaaggcctgaagaggttaaattgtaacctaattacaacgacagtatctcctaaaattatttcctgatccccaattcagttatctctcaactattcttcgttaattgttgttgtaggttgttatttcatgtacacatgcatgcgcgcgcacacacacacatacacacgtagatagtcattagctgcaaggttttcatgttaatgtatatgtcccttgctgctaaattgtatatatacaaaatttgaaaccactcttggagccagaataagcaagcaagtcctagtgataccatcaaatataaagaatagaaacactctctagagatagagttgagatgatagcattaatctagagagagtatttcctgattcttttgggattcatttatttattcagtaaacatttattaagcaccttttctgtgctaggccctacttgggtgtggtatgcaatagtgaataaaacagacaaaaaacactttttcctgaaacctacattctagtcagaaggggatgaggtggatgagtttggaactggttgcaattttagagaggttgctcagggaaggaagtaaggaagcaaggcatgtgggtatctagggaaacaatattccaaggaacagcaaggaaagatgctgtgaagctgagtgtgcctggagtgtttacagcacagaaagagtgtgtatggcagagcagagtaaataaaagggaaagtaatgggaaagtagtgtgttgtaggccaacctaaggactgatttctactttgggtgaaatggaaagccattagaaggttttaagaaggtttaagactttctgacagtagcactgagtacagtgtggagaatagaacgaagagccgaaggtagaaagctggaagaccacttagggagttacaggaataatctaggtgacgcatactggtagctcaatccagtgtagtaatagtcagattgtggatatattttaaagtttaaggctgtgagttcatttggatcaaaagcatctgatggtgaaagagagggaggggtcaggaatgaccaaagacttcttggcctgagtaactagtaaaatagagtttattttactgaaatgaggaagactgtggaaatacagatttggagagaaagatcatagacatgttaagtttcaattacatatccaaaaatagatgttgaaaaagcaacaacataatacaggtctggagttcaggaaggactccaggctggagagaggaaagctgtttttatggtccagatctttgtttagaatcaccaaagagatgtgattttaaacgataggacagttgaatatgtgtagtcagtggaaacagtctcattttctattgctactcgtctttatcagtggtatccttttttttttttttttttttttttgagatggagtttcgctctgtcgcccaagctggagtgcagtggtacaatctcggctcactgcaagctcctcccgggttcacgccattctcctacatcagcctcccaagtcgccgggactacaggcgcccgccacgatgcccagctaattgtttttatatttttagtagagatggggtttcaccatgttagccaggatggtctcgatctcctgacctcatgatctgcctgccttggcctcccagagtggtgggattacaggcatgagccactgcaccgggcctatcagtggtatactttttaataagataacacgaagtaacatctgctgagtatatagataatttaaattgagacgttttaaattgtctctggttcttattaaagattctttaaaacaaaataaaaatacattgtttcaaatattgtttaaagtcttagatatgttttgttatacaggcataatttttttccatgtactttttcttaaatatatggaaatttctaacacagtttcaaatatttagcttaggtctgaattagttgtttatcatctgttcatctgttctgtcttctttaatttctacaccctatcctccaaaagccttgataaaagatatgtctcatatattagctttttttttttttttttttttgagacaaagcctcgctctgtcacctaggctggagtgcagtggcatgatcatagctcgttgcagacttgacctcctgccacagccccctgagtagctgggactacaggcgcattccaccatgcctggctgatttctaatttttttgtagagatggggtctcgttatgttgccaggctgatctcaaattcctgagctccagtgatcctcttgcctctgcctcccaaaactactgagattacaggcatgagccaacacacctggcctaagctacattttaatcaagtcagcatagttatccatcactccaaatgagcatgtccttgtattagtgtccatgcattgtctatgatgtagatggagaggtatagcatattatcaaaacccacctcaaatcctgtgagggatgtgaatcaataaattaatgagtttgttctattgataccaaatggagagagatgagtggagattgatcactatcagttatgtcaaagaggtaagttttaaaccagtgtttgaagttatgtgagagaaaccaaaccaaacaaggtaattaaacttcaaattaactgcttttaattggtcacgtgtgtacatatacatgtttatgagtattttacatttctttgtataataaatcccctgaccaaaaaagatacaatgtaagactcaaggtgaatgttctggatacatgtatttgtagaagcaaaaggaagggacacaactggttggaacctgtatatctttaacattaggtggtaaaatggaatatactatggaaataaataccattgaaatgttttatggtattgccaatttaatacatattttatgataagaaagaagagaagaagttcattgtagattccacatgggtaaatactttaaaaagtattccaaatacagctcctcatatccttaatatatctgttgtcaattattatatatttcatgtcttggatcaagttttcttgtttggacaagaaatggaatggcaaaatccagatctttttagctgaaaacctcacctattctctattcacccacttctaaagtggacacaataattcttaaaatggtacttaaatcagtatagatttgcagaaacaaaggcctgcttaagaatgttctttcatttaagtattatgaggttaattaaatgtgtgaaattgtaacattttgaattattcacatggcattttagtttttccttttaagaaccatggcttttctgtgcaggtgttcttcactgggataatgttttaagataatcccctgatgtcatttaaaggaactcttatttctgaagcatgattgtgtgtttttgtgtttttatagtgtatctctttcttatcctcttttggcctttgtttatattctcattttaccactatcagctactattttctattgttgattgtttttattgtttattctattttctattgtgtatttgttttcacataacaaatcaaagcctgtttaatttgtggaagacagttgtcaaatgtgtaatatatccagttactaatttttctctgattgatgctgctcgtctcctttcagatgagtgccttattaaaaccatctaattttatggtgcttactagaaagaatgtagagaaattgtcaaaattttctattatttactcagtaacccaaaatgatgtttctggaataaaattttggcttctcagttagtgacttggtggtagatgaacatataactattataataatgtaatgaaaaaatatttttctgtcaaacatgtttttttttaaccttttacgaaataaaagttgttggtaagtgatgaaattaaatgccttcttcaatgccttgtggaaaaaatgtcactataaggagccactttttatacagttctaccctgatcaactatggttgtgtgtagatgtgagttttcctttataagcctgtaatttgggacatctgttaaaacaataaaaatttctaattctctttgatagagctggagtggaccagagaaattgcttgctttagatgaactcattgatagttgtgaaccaacacaagtaaaacatatgatgcaagtgatagaaccccagtttcaacgagacttcatttcattgctccctaaagaggtaaagattatcaattacattcttcattgggtgttatttgaatatataactcaacagctaccacctttaaacattgtcatgctaattatggtattcggtattgtcagtttgacactcttaattagtggtaaagagggctgtaaattgataaattgccatgacttaataatgtaattgtcagattaaagaatgcatgtgaactaagtaactcctttccactatttgaataagtcatatatgtgagagtaactattgagtttcactaatttgattatttatttaccaagatttttttgtcaagttatttgaagcccaacctttagttatcttggtatattcatgatagtagattttttaaaattctgaccatattaaaatagcaatattttttctcttattttattatatatcttaagtcccttcaagaccaataggtttatgattctattgaattctagttctctggtacttaacattgtgtaattattttaaatacaaatattataatttcacatgtgtatatttttctaatcattctcctccccatctccattacacaaatacagatttcccatactaattacatgctaacagcacttctttgggaattatttgtgcctatttctttttatatttttatgtaagctgaaactatcatagactgattatatgggcattcataactataaggcatagtttagatatagttcctttattgatattatagacctagtaattttgtcagtgaacagaaataatataacattaaataatcagcatcaccatgatttatattttcataagtaatatgtatatgcccctagttatatgtcagctaagaagacaatataaaatactaacataccatttcaaatgatcctgaaacttcaaatttgaacagaaaaaagtcatttttttaagattatcattctaaatatcctaatggtgttaaagatagtcccaattagaattgcacgttggatacgaataaaatagttgtatcatatttaatgcatggaactaagcataactccagagaaaagcaaagtggagaaaatcttttcttcctaaaatgtgttttctactgagtgctttttaaaaatgactttggcccagaagaaaatttttatttttgctggctcattctaaaactgaatcctttgaatagatttggcttatgaaagtgacgtcagctctctttgcttccagaagggcattaataatgggaaacacttacataagtatttgactgaaagtgattttattcacctttgatatattctctgtagtttacttagtgatatcctccgtattttagaggttatcatttttgttaaaatgcaggctttctcatagtctcattttttctcttatttctaataatggtgaggtttttggtttgttttgctttatttgaatttccataacttcttaacagacaaaatgttgtgtaagatcagaatatgaaagtattttgaaaaatctaagaattaaaaacaaataatcaataaccagggtaaatccagtaaagattctctcagtttaagtgttcttttaaagactgcctggggaaaaataagtgaaattagttatataggagactaagggagagccttggtttagcatatcatctgtgtttccctcttgttttcaactaccagaaaaaaaaattcctattttgaaaaatgctttattgtctaagtggataaataatgatatagaaaacattatcttctcagaaacaaagtattattatttaaaacattattaccttcttgtcaggaaatggacttatttttcttataattcctttggccatatctcaaaactatttacaaacatttactttattaatctcaaaaagcttgacagaagtctcactaatttcacactagaggcttgtgatgaatagcattgtctttataagtgaataccaagaaaatcataatcatacaatagatgatctagagcaaaacctgaaattcttaatagaggattagccaacatttaaaaataattttttttccttgactagtttaatccactgaaatacagcaagtctgggaaaatattggctaccttttaagtacaatgtggttgtcttttatataggttatagagaaaaataaaagcaattcttacagagttggacaaggaaaaggtaattatctgaaatacgtttcaattcaagagcttttctctggcagaagtgatctttaggaagaaatactaatctgggttttacctttaaaacaaatgaatagaaaactgcattaactggaccatttgacttggctgtaagaaacccataactccctaccatagcggttggacagacaagagtctcactctgttgcccaggctgggatgcagtggaatgatcatagctcactgcagcctcaaactcctaggctcaaatgattctcctatgttagcctcttgagtagctaggactacaggcatgtgccaccacacttgggtaatttttttgcggggtgggggtggtggtagggggggatgtcccttttatgagagtactaattccattcatgaggactccaacctcatgacctaattattttccaaacacctcacttcctaataccatcacattgagggttaggatttcaacatgaatttgagggaatatggacattcagttcattttagagccctacatgacaaaaccttgcttcctttgagggaactggtatacttcctcctcataatacgtagtaagaatcatgcaactaattgctattctctttttggcttggcagtttaattcaaaaccttgtaagacataaccttgttgacagcgtatttgtgctctactttttctctagtttaaaccttcttttctgattttttaattcatatttttttgttttgctttgtgtattttctgtattgcttagatgttttgatggaactggctagatgtaaataggcaaacatttaataagtcgaagttcttggttgagttagtatcttcactacttttaaggaaagtgtgccagtaacctgtagagttagcacttcaactttctttagcgccttagtatttctttatatttctctttaatgcactggactagatgatctcataatcactattaatgctggaaattttacctggtgatttggggatgtacttttgaaatctaagctgctctggatttgctgtttcctagtctttaagatacattgactatttctgtaaattacctgatttgtacttcagttagataacgaaagcaaagaaaagaatagactttttctgtttttagataaatattttggtcattaattagcatctcttttgcacaaatatttttatgcctgatagcttctatgttaaattacaattccacatagattagtaatggtaacaccacattactgctatactgacacaaagcgaacttgcctaggctttgattgattggtaattcccaaattaagtagcttttggagtatctgttaagtacaatattgagactatatatgtgtgagtattgttgaatatagttacacctttttattttaaatgactcagcttctctacataatactggtcttctctgatataccaaaagtaattatttcacaaaaagtttttcactatcattcatcagtagtagtattaaactgaatattggctggttggagtattttgtatattgaatatactcaatacatatgtaattaatgatgagttgttgttgttgttgttgttgttgttgttgagacagaatcttggtctgtcgcccgggctggagtgcaacgacgtgatcttggctcactgcaacctccacctgctgggttcaagtgtttcttatgcctcagcctcctgagtagctgggattataagcatgcaccaccacacctggctaatttttgtatttttagtagagatggggtttcaccatgttgaccaagctgatgtcgaactcctggcctcaagtgatccacccacctcggcctcccaaagtgctggtattacaggagtgagccaccacacccagcccatgatgaattccttatatatgcaaagtcattgggaaagcataatttatattatatactttacatttagcatcaaaaaataattaaaattgtgcttttactccctgtgtgacccttgaccctttaacggataatttatgttagttcttgtcatttatttcatatgaaaactgaacttaattgctctatttaaattgttttccctagtatcataatttaaaattttatgtgatatgtgaggctacttgaataatcagtcagaggctataattccccttctttctttttttttctctttatttttcctttttatttatttgaactgacacactgaaaaaaaatcaatagtttataagtattataaacaaagtttttaaatcagtggtataccaaagatttagctcaaatatacaactcttatgtggcccatcaagaagatccttataagtaatataaggtaatcagaactcaattacttttatatgtctatggaatggtttaataattgtccccctttgtcatttacagtcaaggatgtaagtgtttcagaagttcattacaataataaacactttgatttgaagactgttaattatgatatatatctatcaaaaatgatctggagtcataccacacagaatatatacaaatctcatttcttatctgtcagggaagcatttaggtcctatcttcagtgaatttacattgtagtaggatagagaaaaaagttaaacaactaataacattttttttaaaaaattgagttgaggctcacagagtaaaggcaagagcagagacagagaggtttaataagtagtccttgaagagggtaaagagttagtagagtgaagttcataaaaaccataaggtgtgtgtgtgttttttatctggtatatatttgttaggactgaagttactgcctttcataattttagaaatcagagtgcagaattatatttatcaagtatctcatcctgtggagaataattaacttattatataatatatatatttatttaaatatgggtattatactatgcatttttttcttagttggcactctatgtgctttcattcctggaacccaaagacctgctacaagcagctcagacatgtcgctactggagaattttggctgaagacaaccttctctggagagagaaatgcaaagaagagggtaaggttcaaaattgcatagagaatatgtttatactttgaaaagcagagtgattctgaaaacaagtagaaaaaataaaaataatattcagctacttatgataaaatccacagattattctacacattcagatgagccgaatcttaacagctaacccctttaacaagcaatataactaatagcaaatatatctgatttgcttcctatcactagtagaattaataaacaatgaaaaagtcaagatgaggatgacttaaaggaaaccccgaaattatatttccaattaaatatccaaatatttagtattaattcattcttttcataaatgtttattgaacacctcctatgagccagacatttcttcaaggtgggggatacagcagtgaactgaagttctcttttgtcatggagcttatgttcttgaaacctgtttttttccttagtagaggagtagttatcctgtgtattgggagaactgttttttttgtttttgttttttttcccagcagacatccagaaaaatatttttgaaaaaatataaaaatactatgtgcctcagaagcaaaatattttagaaagaatcatagagcatgatgtaggtactctataggtgattattcccttcatggtagttatagtcaagtaggaagcattgatggttagtccagttaataaaggtttgaggtaaagatccagggtgcttggtttgtcatcctgcttttgacctctgtagggcttggattaatcagtgaaatcttcattcttaagtatttgcatacataaagtggaattaatctgtgccttcccttcttccatataatcattttttaaatgatcttttaaaaattgtgataacatgtatgtaatttatcagtttaaccatttttatatgtacagttcagtggcattaagtacattcacattgttttgcaactaccatctgtgttcagattcactaccatctgtgttcagaacttcatcatcttcacatgaaactccatgccccctgttctaactccccattattcctctccacagcctctggcaaccacccttttactttctgtctctatgagtttgactactctaggtacctcatataagtgaaatcatacaatatttttccttttgtaatcagcttatttcacttagcataatgtcttcaaggttcatccatgtcatagcatgtgtcagaatttcctttattccattgtgtatatgtatctaatgctacattttgtttatccatctttcgatggacccttggattgcttgcacgttttagttactgtgaataatcctgctatgagcatgggtgtacagatgactgtttgagactctgcttttaattgttttggttatgtatccagaagtggaattgctagatcatatgttaattctgtgtttgttttggggaaccaccagactattttccacagcagctgcaccatttgcacaagcattccaatttcctcacatcttcaccaacatattttagaatcattttgagaaatgaaatagtgaatacaaaaatatgccatatagaatgtaaactttacattattgctacatactgtgataacagtattggaaatttgacaatgtcttgcatttcagtggttttgagcagagagatggaatactctttataaagatggaatactttataaaatgtataactgttattattggtgaaggcaatttactcttgaactgtaacactttagggattgatgaaccattgcacatcaagagaagaaaagtaataaaaccaggtttcatacacagtccatggaaaagtgcatacatcagacagcacagaattgatactaactggaggcgaggagaactcaaatctcctaaggtaactgaactctgcacaatgtaacagaaaccgttaatcagtgttagtacctagtgtactatttcacttggcaaactgtataatatgacactcctcttctgtagaaagaaaaccgtatattttaaaataactgaaagtattaactgacaatattcttttattgctaatttctctaatctgacaactaattgtgcttgaataaaagataagttaaattgagtatctttttatcttacgtaaatgaaaatctctcaatatcctattgaattttactgtgtcatacacattctacctaagaatttgctgcctcctctgatcacatactccaaacatgattttttgaggagtgtctgtgttttggatttggtgacttttgtgtgtcttatttttacaaattgaagcataattctggttttctgtctaataacatttcaaatacataaagaatggtaaaaacacttgaaattattgggcgtatttttacaactggaaaaaaccaatttggggagacattgttatactaatatgtatatattatgattttttttgttttaagaatgctaataattgttactctttgtggttaatagatgcttattttgttcttgtgtgtcaaagttgtaaaaattagaggcatattcttctggtttgaacagatggaatagattattgtcaaatacattggtgtttgaaccatctttatgaggtcttgatgcagtaaaactttaatagttgatgttttgagtataaataatagactatggaaacaataaaatgtttaattctggattcgtcagccaggaaaaataatgctttatataaatttaaattctagagatagactacaaattactgttcctgtttatgccttcatttttctcttcaccagtattttaaagaacataatttaattaacatatttctaatctgcacatctttcttataggtgctgaaaggacatgatgatcatgtgatcacatgcttacagttttgtggtaaccgaatagttagtggttctgatgacaacactttaaaagtttggtcagcagtcacaggcaaagtaagtttactcttcctacacttcacaaagtcataattttgtgtatatcattgcaagtagcaactggaaaaaagaaaacaataattcatttttgattcttagttatttggatagcaaatatcagagctcataacttagcttttttccccatatttatgaaagccaatggcatgaggatagtgatgcttctcatgatagaggtttcattaattttttttcatttacatagtttgtctatctttgtgacagatactttaacattttcaagggtgctttatgtttttcttggctcaatttttccccacttttatttactatctaagttatacctcattactttgtaatgtttttcaaaagtatggttgagggatatttatttttccttataaactgctttgttgataacctacctgtagtttagaacttaataaatcatgttaatttatacttatctcaaacccttgtgagatgagagtggacaaggcatttaaatgttaagtttgaagaaaaagcccaagcaatcagcttgactgtgtaccatatcataccttaatgattctctagtgaatatttatttttatctgtccagtagagaatattttcatgaattaagtaattgccacctaaaaaataacagaagagagtatgaaaacaaaaaaaaatccatgtcttttaaaatgtcttttaccatgttgcttcttggcaagtatcttataaatattccaagtcagatgttaaaattttttaaagcagctttatatttccatagtgtaatattagtttgggatttattacagggctttaatgtctgtctttacttgtctagaagtagcttcactggctcctaactactgccctggggcagattcagatgaaatgtggtatgtagccacacccaatactgttggcatactttcattgaatctattcccatggaagaaacccagttgcaatttcagtattatttcatcaaagaggccaggccagagctatcataactgctttcatgtcgtgtttcctttttattatatcatgttttatatcatatgtttgctttctacatactacatacacaattgcttgtgcttatataaatttataatgcttttgaataaacaaaacaataaatagtgatgggatcattttatacggatgtaatattttctgaagagccaaacaaattaaataagtaactttactccaaattttaatgagctattaaattttatttaaatcacttttcctttctacccaaaagtaatcatcttaagtgtttttccagtgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaaggtaagggaaaatcttgtcatggttgggacttcttcctctattattggtgaatttgggccctttctgtgtagagttactgaaatagtgacagtgtgctcttaaattagatttaattattacacttcagatgtttctattctaggaaagccatagcgttttgtcttaaaattggtgattgtctgtaaatttcctttctcatttgctagtgttgtcagcttcctgtttgaaactctctactctctaatctctaatcctttttgttgttctgtatctttgatgactcatcatattcaagcagaaatcatgttttctctctctttttttgatattcttatttccgcaagtgggacaaattcaggcttcaagcgtctgagcctttttaaattacctacccaccttacacccaccctatctctaagtctaggcaaaagctgaggcctgtgggtaatttgttctctcattctgaactattgtcacagctttctaacttggttatcttatgttttatacagctgacatatagtccttttataattatgatagtgtcactgcccagttcttaagtctttttgagcctactgttgactaacgaattaaggaagaaatcctcgatccagttttcaaaatggtttctgccaaactggactttttgactttctcttctcagtgcctttgctgttgcttttatctcctcctgtaaagccctcctattaaaatcctatctttctattatcgtcatcatacccctgttaaacaagtgtgatttctacctcatttgtcaaaaaaggtctctctgaaaggtccttacagtgaatccatggcagtctttacctttaattctgtctatcgcatcttctttattaagaacaattacattaagcagatttcacaccagatttgtaatatgtgaaaaggagtaccttttcaattgtcatttacctctactttgaacagtgtgctatactgttaaaatttgggggatgagtatcaagagaccttgtattccagtttccattatgttctctcaacattaggttctcaattaaagggttctctcaaccctttaattatctacttttttaaaatatggaaactgtggtatttctttatctcttcagaatgacataaggatacctgagttttcagctttgttttgtaatccaaaagtgattacagtatttgtatattgctctggttttcagaagagtatagcgctgatgttgaaccaaatgtttaagggatagccattgtttttaaagatgtacatatcttttaaaatctttatctcttaatatttttatagcctattgcatatttataaatctaatcattaaaagcataagcagcctgtgtttgaggacatgtctaaaactggttaatttttactggaatgtatagtgtaaacacaaatctgcagctgtttaaattccaaattctgacttgttttgttgtataaactttattgaattttttacctaatcacaagggaatatatgcattcataattgaaattttggaaaatggcaaagaacataaagaaaatttaaattctttaaaccactaccacctagagttctcccattaacattttatacatatgcccacattaacattttatttctaaaatcaaaatcttattgtatatatttagaggcagcttctgctgctttttaaaaaatacagtacagtggttttcaaactcagtcccacagaaccttaaggtgctgaacctaaggtccccaggtcagggaaccaagcatgcagcattctaggcttcccaacttcccattcccttattatgtttatataccaaggttttacataggatttcttttgaaaatggttgttgctgtgtaaaaaaaaaaaaaaaaaagctaaaaaaccttgactaaatctaccatgttttctcatattattaaaaattctaaacgtgggtttttttgttttgttttgtttttctgtttctccctctgcagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacaggtaagatctcttatctctcccttaaatgctctcctgatgaatcataaggttgttttactcagataatcactgtcaaattgctgatccagtacagtccaaaaaagattaatcagattattctgctgtttttatatacgttaaaattggcatgaagaaagtgtaaatttcaaaattatttaaatgctttcaattttctgtgatctatttctcctctaaatacaaatatttgttttgcaacctgacactttggggctctaagttttgctatgttaattaatggttaaatagtctcataggagagtaacctggattttaagaaactcctttgcagaagattaatatggccaaaagctgcccctttgctttgatagctgaaagttgatggaccatgtaattgtgattccatccccattcaaagtggaaatagatcctttctgcataaagcactctactgtcccaggcccactgatttgtagtcataaagaagtaaagttgactaaaccgatttcttttttttggtttatatttcaagcccatatccttgaaaacattgccatattttgagatcattaactagatgaataggaagtttagaatatgaatcttgttttatttccagaaaggcaacatttttaatgtaaaaatatttcctatagcttatatagtttatttaccagttattacccattaatgttcagcaaattagtacataatcttagtactgtcaattctgttcttaggatatttgtttctttgtcaaagggatattgtccatgtcatgaaatattgacaatatgataaaaatgtttgctaaattagaaaaggtgatgaatttggaggaaggggaattggctgcacctgtttctgatatgttcagaagcttaatgaatataatattctaatttaaataaactgtttgattgagaaaagaggtagccacattattgtttagaaatgatagactgttattgacttttggtgtagctgggaagctggagaagaggtagtatgtagtttgcttttgatttcaaaatgccacctcttctgattccagatacaattatcttttggcacatttcctaattagcattaggttcttataaatgaaattttattttacacacagtttttaatggaacttacttttgaacatcacgaaagttatctctagcccttttcatgccttaggtgctgataagcattccgtttatcataagctatgtcattagtctcagcttcctagtgggaagtaaaactcatagcaattctctcagtcatccatgatatatagctaggtggggccagatgatttgaaaattaacatattgttcattttaggtgctttgttttcattttaagttgtttctgcatctagtttgaagctgcttggcataaagatgagcctttctgatagaagtgtgtgagaacatacattgtagagttgcttgatggcatgcactttatcctcattgccactttatcttagtacctcattttgttcctggcattacctgtttcccatcctcttccccctttcctactaggattaaggtcagtaattgataggaagagtatccatacttctattctaataaactgtgtcttttgtagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtaagtcttactcacctttcgttttgttcattaaaaatgagatcattattcaagagaaatatggagccttagcatcacaaaaagagtcaacctcatacagaatggttacttcagaaaagtttatgaatattttcaccctgacttacagaactagtctactcttgaggaaatgttttaaactgtgtttttaatatgtaatataaaattgtttaatcacttcctgcatgttcaattgactctacttaacctttaagaacagtgagaagaaagatcttggctatatcatagaagcaacgtactcattatattttttgattgaccattgcattttgaatttgtagtccaattttaaagggctaccttattgaaaaaaacaacagcatccttgatagaaaatttatttggcctccacttagtcacgtgcttaactggaatggagaggaataatagtgcctactaaagtagagaaagaaacaagtgaaaatcactaatattagatgaggtacagcagtgcctcatgattaactttttctatttgttatagcccttagaaagtgtttttttcagtggggccatctgataggcaacattgcagctttttactcattggttttgtctctcctcagagttttggaatagtgactgttcagctttgagtggttcatggtggtggggtaatagatctcttagtgctcatttgtccatcagcagaaggtatgaagttgcactatctacatcctcaccaaggaatgctgtcgttcacacagccagaaaataagatgtttgtctggtagctaaccaagcttcttggaagacagacctcttgaaagttaatgtttcagttacaaatcactaccagattttcttacatccatgctgatcctaaaatactgaggacatgggtttctaaatatgtatcatttcaaatgttgcatttattgtatgttctaaagtagaagtctacaaattataatgtaactaactcatagccattatttctaaccagtaattaaattcttttggtttttgtctaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattgggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttttttttttgccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattatttttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgtttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgtta

SEQ ID NO: 4 - DNA, 163622 bp, Mus musculus
gagcgagcggggccgccaccgccgcctctatcccagcagcgcggaagagacccgggtagctgcttggtggagcgacgctagcaccgcttcttcctcagttccgcgcctagccagccttccgcagctgcccgcctcagcccgaaggaggaagggagccagccacattgtcagcgccaccgctcgactctgaagcgtgagtggacccgggggagggggacattataaggtgacaagtgggagcgggtgggtgctttgatgctggagttcggctgttcctcgggagggttggcagtggaaacctttcctttctaccaataggagaaagtgtcagtttgcaggagtaatgcaagtactcggagttttttcttagagcagctgtgtctccagagtggacacttgcacctttttggacactaaactgagaaagcctatggccagctctgtcccatgccttttcctggcaaccttattctggcagtggtttttgtttcagggaaactcccatggattcccacgtgggaatcacttgaatcaacccttaatttttttttttcctttcctaactattgtttttgaagttgccttggatgggaagaccagatttcattttagtgcataggggagtttggtgcccccaacgctttatttcccaggtctacagagtccgatgtgtagggtgcttgcatgtttcagttggctcaggaaaatccgagttcctaagatgcacaacccactgaaacttatttcagggagagcacttaataaattaccatttcgtaaaagttgctgcagcgctctctctctctctctctctctctctctctctctctctgtgttgagggagagagggagtaatatgttttaagtggtagcacatttttactaggtttctactagggactggaagggcatgcatgcagctgtttttatttggctctacttgtcgaccttgagactttatactcttttaggaaaacatttagcactctgagtgttaaattagaggtatagaaaagtaatgctcacatatgtaggttaagctgaccatcctaaggcttaacgtactgtaggatttcatgtagcaaggaacataggtagtagtttaagactggaattctggtctatccagacagctaaatgtgggcagaataaagatgctctgtagaaatgactagaaacttgggagtatcaatttatctacccatccatccatcttaatttctccattgagggtcatgattaaatttgtttgaaagtttttcacatttcatgtttgggacctgttctcccctcctctttttaaataatgaacctccaagtttatcattctgcattatatagcaatgtatttctctttgaccagccttctcaatctttcaggatacagtttgtttgaggaatgctaaaacactgtgaatttaccttaccttgcttttatagattttgtttaatctctactatcatctgtcccaacttttcttttcttcaagactgtttttctatgttaccttggctgtcctggaactatctctgtatatcaggctggcctagtgttcacagatatgcacctgcctctgcctcctgagtgctgactgggattaaaaggtatgtgccctgactgccagccacgtcacagcttttttgagtagctccctcccccaaccctcatttctttggataactttgccctctttttggatctcctagccttttttttttttttttttttttgttagatactttgttcttcatatgatttcatgagacactttaaattttgcttaagtgggaattagtatgaaaatattgaatggtaaatttaactttagatttagaataaaacgtgtttttggatctgctaaccttactttgctaccctgataaagtcatatattccagttttgaaaaagatgatatttgagaaacttagactttaatagtttgcttcttggaaaggaggagagtggtactttgagtttgtactgcatctaatcagaggacctgctttgtgtgtaaattggttttaaattatggaagtactttactgaatgtagtaataacaagacatttgctattttcagtcttttgagggtgacagagacaaccaactacaaattgcacaaaactttaggaaaggttgaactggtgggaaggctctgtgggtaaagctgcttgttgcaagtctaaggacgtggagtctgtctaatgcccagaaccacaggtggacagagaaactaaccatacagagttgttctctgacctgtgtcgtatgtgtggacatgtggtggtagtacagttgtagtagtagtagcagtagtagtagcagtagtagtagcagtagtagtagtagtagtagtagtagtagtagtagtagtgtagtagcagtaaaaagaaatacatttcttaaaaatgtaggaaaggggacatatattaccattattactcataagatcactctgtgtcagggaatgcagttgcacatttttattagggaaatactgattttggatgacccaggaataattggagtgtctgagccaaatcttggaaaggctctttcaggaagaaaagaacatacacaaacaaaatgttaagggcttagcgtcagaataagtaaaagatgagatctaattaggaagttagcttggtaggaatgcctactgagggagggaagttcctgactagagttaaggtcagtttgtgcttgtataagagaatttgaaaacagtgttccaaagaaggtagtttcaggatattgggagaaattatattttaggacagacattatactggctttaggattttgtttcattaggagactaacatttttatattatggtcatctagggggcagcacagaaggagacacgaaaaaggaaatgttttgaatgttttttagcatgatgctagtttcttatgagtaaatcagaaacagggtagcaggctttgctttggatgttgaatttggggttaagagctaggtatctgtacagttcatgtgttagctcattgtttttcttttcctccctctctctttaaaactttttccactttctttctcttcttttgtctctgtctcttgtgccaggctgaccttgaacttgttgtgtaccttttaacttgtaaccaccctgcttggacttcctgaatgctaaatgtactagtgtgtgctgccatttcttgttttgtagtgctggggatggaacctaggcctttgtgcatgctaggcgggtactttaccaactgagctacatccttaggacctaccccgtgcagctttcaagactgcaaaatagtttgagtaatatgctgacagtgtaggtagatgggagtcatttttaagcctttatatttctgagattactgatacgaaattaagagctgaaaaatgaatttaaaattttgggacctgaagcagatttgtgttggtataagcacagctatcaaaaggagagacagtccccagaagatcagcttcgcaggtgagtgggaatgacagttggaaagaggagcagtctgtaacataatgaccattcagtcccaggacatgggattctgttttagaagtagttaaaggttttcaatagcacatttagatcctgagatagataagttcagaaaacataggaccttgttagggaacttataaataaaatggaaggccattacagtttccctgcattttataatttttttccccgagacagggtttctctgtgtagccctggctgtcctggaactcactctgtagaccagactggcctcgaactcagaaatacgcctgcctctgcctcctgagtgctgggattaaaggtgtgcgccaccactgcctggctcattattataatcttaagcttggtcttaatactaatgaagtggggctaaacattgatgaaagggaaggctgtttctcttacttcagaaatgtgcatctttgtgtccctacaatgtgtgtttaggggggacatccctgtgttggtacagaagtattagggatacttttttgtataaacttaaaaagagcctacaatttaggaatatttaaacaacacaaacaaacaaacaaacccaagctagataatgatggcacatgccttttaatctgacccaggtagctctgagttcaaggccagcctagtctacagagtgagttacaggacagccaaagcaacacagctaaacctggtcttgaaaaaaacaaacaaaacaacaacaacaaaacgaacaaaaaagactattcaggcatctttatggttagcttttggtattggctttgataaggataaaagattgtgaataaaaacacgtaatcattatgatcctttgtttgaattcattgtttttctttggcttcttaagagagcatccagttggcagtaatctcttggaatagatatttttaaacaaggatgtgggtctacaattattttagggtgagtagatagggattggaatatctgcaatataggggtcttttgctacttggcccatttcttccatgggtgaagacatgcttaagtgtatttaaaagacagccttttaggattaacgtgatttactaatatggcgggattctgatggagaatggtaacatcaatattctttgccttgatagttatgctatttatatgagaggctggaagttttgtatctttgtgtgtcttagtaccagaggactccagagtctgtgtgagttatcagcagttcatttagatatactttgaaaatgattctttgaaaagtcttgtgggacgacgtaaagaatgggttggcaacctttctgtgtgggtcagcatactgtgtcacaactttcagctgtttctgccatgtgaaaacagctgtagataatatggaagtgtgttataactgttccagtcagatctcatgcataaaaacaagaagtagatgggatttggcctgtaggcggaggtttatagctccctggcagaggacagctaaatctaccagcatctactagtgtaagctagttatactagaaagccagattggccatttcaggtggattttgttttctagatgagatgtgcttctctggactgccattgtaatgatgatttgcctttatagtaatgagaattatggatcaagaagcagagacttgatgttctgtaaatagatctgaaaaatagagaccttcatcttaccagaaggtgccataattgatgtcttagttacttttctatttctgtcacaaatatgtgaccaaggcaacttactgaaaaaaaaaagcacttaattttgcttacagtttcagagggtgagcccttggtcatcattgtgggtgagcaggcataacactggagaagtagctgagagctcacatctgacccacaagcaggagagactgggtctggtgtgggcttttgaaactgaaagcttaccccagtgacacacctccttgccttttcaaagcagttcaccaactggagaccattcaaatatgtgagccagtgggggccattctcattcaaaccatcacattggtatgtagggatacacttgttttgtattatggctaaaagaatctgttaaggaagaagttaactttctaccccactgctacagggataggagccccgagaagctcgaggcctgctaatcaagcattctcccactgagctgcatctctctatggtataagcttacttttgaaaggttctcaaaacaaagtataaagcatttgcttttaatgtgaaagtaaaaaggcatttaagttgttttgaatcagacaaagatgttaagtcagactcaaggaaagttaaggtttaatgcttctacctcttaaaatcataatctcatactaaatcagtgtgaagaattcagtacagaattttcattttgttgttaaatcttaaattacctaaaccacttgaatgtatactttaaacaacattgctatctttaagactaatatagacaaataaatgtactacctagaaagaactagctattcacttagtgaaatgtttaatgttggcttaatgttaaaactttcgtttcatatgtcaacagaaacctgtattagatgctagtatatttaccctggttttacaaactttgtttcatggtactgcaattttttttttttttttttgtataatacagtgcaatagtgatccctttttagttagtttcagaactgtattaactatagtgatccctctgtataggacccctgcagataccaaaatctgaggatgtttaagtctcttatgtaaaataaagaaatatttaaaggaattagtgagacagcttgacatcatgtctgagaacctgagtttcatccctcagacatccctaccatggacatgatggaaggagataacttactcttgaaagttgtcctctggccttcatatatgcagtgtatagtgtgccaaccctgccataaataatgtaataaaagtatttacatataacatgcttatatatataaacatatatatgtatatacacacatgtatatctacatacctctttctgtgtactttaaaattcacctctaaattacttgtaataaatataataaagtgtttaataattgttataatacaataaagtagcacattagcagttgttatgctgtgttgtggagggataacaacaacaacaacaacaaaaatctattaggttatacttttttttctggatatttttcaatttggttggttgagtctgtagacatagaactcacatggaactgtttattctgtgtatatagtagatactttagtttctgttttgctctttgaataaaagatgatataatctgacaatacttttctttttgggggtggggtgtaggctctttagtttgtatgcatatgcaattctgtgagacagtctttcctgcccaggctgcttttcagtgtgagatactgctgcctcatcttctggagtgttgagattatagggatctataactatgcctggttttattttggtcttttaaaaatcttactttaaacattttgcttttaaaaataattttatcaaaagatgggtgatgaagttaaatgacactggcactctcatgtctgcagggatttcaagtagaaatattttactttggcaacacttagtagaccaggaatggtgagctttttggtgtgtgttcattaccattgcaaatagatgagtacatttgttttcaaatagtgaactcttgccatcacatatttctattttgagactcttctcatttagtataaaatcattctctggtaggcttgaaaatagacaattagatttttttgcagttttctgatgttacctcactgatagtttctgtaatacctttcatgtatgcaaatggagtatgtttcctaagtgcatatgacatttatacatattctagtaattacatgtttacatataaatatagttaaaattattcctaagacattttgagattgtgaaatccttgagaaacatttgcaattctcactgagccctgggacagtagaatgattaatttaaaagaaaagttttataaaatgttcctttacgttttttaagtacactcaatttctctaatagagtaagcttaaaaaagagaagatgagaagcaatatttgtgttgtatatcttccttgtgtgtaggtggaaatgctcaagagatgagctattattatttgttctttctcctaaagcatagctgtgcttcagtcaggtaggtcctgtgtgctctgctgctaatttgtggcctctgccctttgcttccacctgaagaagaacaactactagaaatttgggtggctttggaaagagctagttctttttccctccctgtttgtgtatctgtgtactggtataatatgataaatactttttttctgtgctgtaagacttctctgatgtttgtaaatcttaaaagctagtgtgtaatcaggctaacacacttacactttaccttccctctgagaagtctctatgaacacactgtgtttaccttgggctttctcagcccagactagcctggaactcactaagtcatttgtataggcctcaaacttgagagccctttgtctcagcttcccaagtgctatcagtgctgggagtataggtctgtgccgccagagtccacttgccaatactggtaacaccaatcagtatgattatttacaattggttgattcttttaggatgtctgtttttttaggagtttaagaagagatagaacttggcatggtgttctaggtctatgaatttgctttaccaaaagtattgagcaatttataactaactacaatcagtggttgtattcctggaaccagcaacaagttgctagctcatctctcatttacctacgaacacacaatcacactttgggggcagaatactcaggtaattgagaacccatttcttagcttcctcaccttgaacaagctacttcctcagtacccactcactggggaaagcatgctctccacaaggtttgctgggagaagctcatatatatgatttaccaatgttaatttactaaaggacatgccttttaatatgtaatgtaagtagaaggcccgagtcagccattattttgctcaacatttctgttttacggtttctattgctgtgaagagacaccatgaccaaggcaactcttataaaggaaaacatttaattggggctgccttacagttttaggggattagtctattatcctcatggtgggaaacatagtgtgcaggcagacatggtgctggagaaggagctgagagttccacatctggatccatagaccacagaagtgagtcctacactaggcctagctttaacatttgagacctttaagcctgctcacgtacatagtgacacacttcctctaaaaaggccacacatcctaatagtaacactcattactgtgggccaaacaatcaaatacatgagtctatggggaccattcctattcagaccactaactacaattcacctttaacaccacccccgagacagggtttctctgcgtagtcttgactgtccaagaactagttctgtagaccaggctggcctcaagcctttgccaggattacaggtgtgtgccatctctgcctggctgattctatccaacatttaaatgagcacttgagcttgtagtcctgtttcataagcatcaaaaatgaatatatgtttcattaacattttattaaaaagtcctaaaaactagggaaatgacagaactatttatagacaaagatttaatcaaatatcactggacttgtagttaactgccaaagacctcaagtggtgcatggaacagaaaggtttcctctttattctggaacaattagtgttttccttttccttcttctgctagtgatgatgatttgagttgtccaggacaaatcctgggatgcgattcagttctcagttcactgataactttactgccaccactgccccttcaacactggaaatacaaagctttggaaaatcgtaaagctggatgcaagatttatctttctcagttattcagactaagattgaaataccttgatttagtgaacacattgttagttgatacctaattttattttcataaaatgattgtgtgtgtgtaagtaatgtgccttaaatacaagatagctttttcttcaagtgtagaatagtcggttttaacagcatctacaaggaatagttgcttcttaatggtttaaagagaggatttatgttattttaaattctgtgtatagatctgaatgtctgtgcataggtgtgtgcacttatgcaggtgcctggaaaggccagaggtgtcagctcctctggagctagagtttctggggattgtgagccacccagtgtgggtgctgggaacaaactggggtcttcagaaagagcagtgtctgcttttaacatccaacccatctctgactcttgatttttttttttttttttaatttggaaggtttttctggtttgaggaccacttggttatgcttttacttacattttacttgacagaattatacatttaatttataaaagctcacaggcaggataaaagatggaaacatttcttagcagtgatgatgtttatgctttatgctccattcttcatgctttattttctcctttgtcggcagctttcctttagattgtgatttaaatgcaaaatcaggttgcttatctgcacgtggatctcttgttagattgtcaatttgatgactatctaatttttaaaaatttcaatttttaaacttttttctttttggtagcaattatagctaggtcatctctagttttctttccacttcagttattttcaaaaaggatgctgatttctataagaacgcagtcacctagccttctgtgtggtcagtagctctgagctgtgggtttgatgcaggagtgcagggcgtttctctacggatcattacacagtggtgttgttttcattaaggaagataagattttaatttactggattgagaaatttggagtaggaaacatttaaacttgttataaaattcattgtgggggagggggaggggtggacggtacagggaagggacagacagcaggacagaagagggagggctgaaaaataaactgggggagcctgtctccggaagttttgaggtcagtcttagtcagaggtcacaccctgttatttctttgggctgtggacctggggtgttaccggttgttatctgctatactttgaaatgttgattagaagattgatttttgttcttggcttttccacatctggattcattaaaccgttgaatttctaatcctttttacataatcagtgtgtcctcgggtggtagaatttccactatacctgataagagaaggtttggacaaaaggtctgatctaccctacatttctgatttctgtaagagggatgctaataattgtatataaggagctaagaggcaagctttgagctgcttgctcaagttcattgtgcttcctgtacaactgtaatgataataatagtacctacagcacttatggtattaggaagactggcattgcgcatgcgagcctttagcagtatctggctcagtgcccactaagtgctagtcactgtagtaatacagagtgcagtgttacgttattgttgtatttgtgaagttgctctgttttcaaaaactgatgtcatttgtgatctttgcttttgaatatgttcctattttgaatactgtctttattcagcagaagatatttactgaatacctactgtgtcaggcattgatctaagtgaacaaagcataaaaatacctgattgctgaaacttatctctggttggaggaggcaggacacacacaataaagcaaaacagaaattgggaggcagaggcagttgaatttctgagtttcaggccagcctggtctacagagtgtgttccaggacagtcagggctatacagagaaaccctgtctcaaaaaaaaaaaccaaaaaaaaccaaaaaaaaaaaaaaaaaaaaaagaaagaaagaaagaaagaaattgggctggtagattttgtgtattggaaaccaccgttaatgaaacctgctagtctttcccctgcttgtgttgctttgctttatttaggaagcacctgctggcctgtccccagctgtctgtcttccgtggaaggtagcctcaaggctttcagcattgccttggcttactgcttacggtggctagctcagcctttgtcttccagtcctccacctctgtttgagtcttggctttgcttctccccagtgtcccacctttggcctttcaactctgtcacactcaagagacagttcacagaaaacctctttttaaatgtatttcatgtttaagcagcattttcccattttgtgtttcctcccttttcattttctttagctaggatagttagcttgcttgccagtctcatttaagaaatcctatttttagccgggcggtggtggcgcacgactttaatcccagcactcgggaggcagaggcaggcggatttctgagttcgaggccagcctggtctgcagagtgagttccaggacagccagggctacacagagaaaccctgtctcgaaaaaccaaaagaaaaaaaaaatcctgttttttttttttttttttttgtatcgtcaattcagttgtgaatcttttaaggttacagaccttttccattccgagctttcttaacttaggaagaaaggataatacttgctccaaggagtgttgtgagttttctgagagttatgtagccactcgggttgctgcagctctggccttagtgctcaagagcccttgtgtctgtcatcccgcacctagactaggagagcttacagtttcacttccattttcttaatgggtcacagttttaggtttcttttttttttttttttaaatgatacaatgttacttttcaaaaatcacatgaaaatatgatgatgtaacaatgattataaagcaggaattcaaggcccatcatgtgggtgctcacactgattgtaagacctttggcaagttcctttctctctcttcagtgtatccatatgttagggaaactaatacctaccttacggatgcttttaggaaaaatgagatcacaacactatctggatgtagtaactaatctctcacagtggaggtgtttctgatggtctcagctatgtggagagaagacaggaggagcagaggggacagtgtgggagaaggaggagaggaatgaattaactcactcactgctgggatctaatgactatagaaactgctataaatgatatatagggcactattcaaagcaaaaaattatctggtttaaaataccattaatgccagtatcgggaagcctgctttgtaattaagtccccactctttcatttaggtcagttttttttttaatatattttatcagttagattctgtttaagaattgattgttgataagcataaacatggataggacttatttaatattcattctaagaaagacttaatagggaagtcaatagataaaactttttaaaaggctttttctgaacctggggaaagtttgccatattcctgaaggttttattaattttttatttgttaatctgctcatgtaaaagtctgttgagaactgagggcttgtgagagaggagtgccagaactcgtgtgttgtaaagcgagcaacgtggacactgaaaggaaaagcacacatccaggttacggaagtgacctgatggggaacttgccttgtgtgttctttttctctccttgtcttgttttattacttgacaagcaaaggactttactgtctgtcttctcttgttatctttttctccttgtttaaagttaaatcttgctattccctgtggagcctttcctttctttgctcttgtcctttctgtgcattttcacctcaggggactgggctggtagcctttctggtgcagatcctatctacatgcagacttcctgtatcatcctctaattcctgggagactatcagcctaaagtctaaagctttaggagttgggaggagcagacgtttctctcttggtgtcgcctgtgtgctaagaaagtagggacataaatttcagtctttgtcattcacttgaattttatcctttgtatgcaaaagacaggctgtaccttcaggtgtctaaaacatatgcatactttgtgtgtgtgtgtatgtgtgcgcatatatgtgtgtatatagttgtctctcagtattagttctagaatctcccaaggctgctagactctgcagcactgcttctaaccaactgcatcactttgaataccttgtctctaggttacttctaactaatactgtgtgaaggtgatggtgatgtaaacaatttaaaaccctatgtttaggacaaaaaaaaaatgtgtgtattcagttgcacaaagcaacttttctttgtgtgtgtctactcttgcatatgtgtgggtagaggccagaggttgacatctgctttgtcttacatgtttttcttattttttgagacaagtctgtcactgagtctggagctgatccattgggataggctgactgccaggatgctctagagacccgcctgcctctggcttcctactgtggttgggcttcatgactagtgtttaacattcctcctaggcattggaactgtctccatgcttgcatagcagactgttgtattgattgatccatctcttccagtacaacatcactttatagactattgtttgtttgttggtttggtttttggtgttttttttttttttttttttgagacaggtcttctctatgtaacctttgctgtcctggaacttgctctgtagtccaggctgacctcaaaggctaactttttatttctatctttagtgtttcttcagttaaactgaggatgttgaatctgtggatttggagcccatagatgttaaaggccagctatactttcatgtatatttgcttctgtaccctactattctctgcctggaggagtctttcttttcttagatcttacttaaaaagtcatcctaggaaacgtgccctgattaagtagagtgagttattttttacttacagtgtctttagcgtttacagaatcagatgtgagtctgtttttctctagtctctttgagcataggggtcatgtcttatttactttgtttagtgaatttccctcccccagataccttaacattactgctacataggatgtgtatggcttgtgaacaaacatcagaagagaacacacagaaaatcttttcaataatctctaaactggaagagattagagattattttgtcttgttatctgtctattcaggtgcttagtagtgccttctgtagaggtattctgaaaatttttattgggtcaattagaagcaagtttcatatgatatattttttttatttgcacctatgtgtatgtctctgtgaatgccacatgtgtgttagtgcctgtgaatgccagaagagggcatccagttctgtggggatggagttatagacggttgtggcccacccaacatgggtattaggaactgaacttggggatctctgcaagaggtgccagcactcttaaccagtaagcctgagagggaaaacttcatagtagatattttctgtgtgactttccttcttcctcatttctaggaagcatgtagtactaagacagctttgtacccctgggagaatgtgccttatgctggcttttcttgatttagtaggaagattttgagacaacacaatcagtttaaacttttctttaaacttgtctgcctttctggggctggagtgatggctcagtcgttaagagtattggctgctattctggaggacctggctcaattcccagcacccacatggcagttcacaactgtaattccagttccaggagagccaacactcccacacaaacatacatataggcaaaacactgatccacataaaacaagtaaataaataaatctatcttaaacaaacaaacaaacatctgccttttgtctaagagattagatttctaattatctttcagatattgcctgttcagaaacttttaccattgccaaatttttcatagcctgcatgctgagctgtgtaatccccgatgggataatgacccatttaagaaactgatctaaaatgggctgaaatgatgctcttgattgatttcttttttattcatggtccttgtctggtgttgtgtttgttgaaaaaacaagaatgttgcttattagccttacattatataagagctgttattaaataataataatactaaatgaaacttgaaggcaaaccaagtgaatatgttttttcaaatgttggccatagctgaggtattgaggcttgaagtaaggaagtcgccactgcatctctttccttttgatgatggattttagccttagacttgcccaagtaatttacaggtagttagctggaagggagaattatgtgaatctaggggataggcatatagatttgatcatctaataaatgaataatttgatatttgtcaccacagtcatgattagcacttagtgagtgcttcctgtgtgtcagtcactgtgctaaggactcccccacacagtgtttacagtttacttcttactttatctcatgctgtaggaagaggaaacattgagagcattgaacacaaggctaggaatttgaactcccagaagctctgtaagctgcactacacaggagcaaacattgtaggatttcactcttagaagcagattacaggctcagaggaaagcagttcccaatcattgtaaaatttaagtctgtatttgaatgactttccatgagccttagttttcagtgtcacttttgccaggttaccaagacattttgcttcacttgcaggagtttattaaggtaaaatatatttaaaacctgaagcagggatgcagtgggtgcagttgagtggtagtgtgcttgcctagcacgtaccaggcacctagcactgccaggacgggcgtagagtgaagggacttgtcaattttatattcaattcataattttaaaaatattaattaactaattaggacaaggtctcaggcagtatgagctgaccttgaactcctgatccttctacctctagttccccactgctgggattacaggcatgcactggtgttttcagaggaggagatgaaactttgaaatatcatctatgaattgaaaattatggttggatatatatgtgaagcatagcagaaatttgagaattaatacttgttaatctcttgaatatgttgtttaagttagtgtggttctgtggaatatttccctaatgttacttctttttggtcaattaaagtatgtcactttttctattctatactttgtttagcatgagtccctactcagtatattattgacaatgcatttaagaagtacttctgatatctcccctagtatgcctttgtatgttaactgggaggcttaaaagtttcacattttaattctttttccttacaagagctaagcctggagattgttttttaaaaaatattttgtttatttattttagtggtggaactcaaaaccaggccctgtgcatgctaggtgggcagtccaccactttttagtaaaagacttcaccgatttgagaactttaaattctgtgcttttataaaatatgtatcagaggttacatgcacgagtagagtatctagtgatttattaaagattaaaaaatatcaaaatggggtggctggagagatggctccacagatgagcttgtatgtatggcttttcctagattcagcactcctacccccagctcataacctatatctctctgctggggcaccagccctcatgtgtacatgcccctacaccacattgaaaaataaaataaatacgtggggaagaaggtctgaattggtcctgagattctcaattggtacttgcttccaggtcttgcaacgttgtttaatccctagaactgacatggtggagagaactgactcccccaggattgtcctctgacctccacatgcatattgtggcatatgtacagtagcatacaatcatatatacatacccacagcaaaaaagtaacatcccctttaaaaatgcataaatggtgctgagttcagctatggaagctagttgagtgattgaggcagagagagtcagagtcttaagttcttcacgtccttggacattattgacctttctaccacaggtttctttcttaaagtggctgttagtattggtattgtagaaatccattcctagaaacattgctgaatgtttaatgccagatactgcagtgtttatccacctctcacatactcctatttcatagtgcaagctaggtgtgaagatggtgtgtgtttacattttcaccttgacttcggagttgacctaggatcatgtgcaggagacatcaatggctaccctttactgcctgcactgtttgccagtttatttcttatgtatttttaatagaacccagcccagcccatgcatagaagtgggggtctcttttcgggggcagttagttatgtattctctaagtttggcagcacatgcataattcttccttttactgattttcactatttatacattgatttatttctttgtatgtgtgcatgcgcgtgcatgtgtgcatgtgtgttggtgcctgtgggtgtatgtaggcctgtgggcacatgcccacaatgtaggtaaggaagtcagaggcagcttgtgggaggcagttcctttctttcaccaggtgagttccaggggtcaaactcatgttgtcagccttgggaataatcaccatcttgctggccctgtttttcctattacttagtttcagactttatatacttgtataaaaatatctatctatctatctatctgtctattataatagaataatatataaataacaagatatttcctaaaatttcttgaattggccatgctggatctcaagcagagttttttttttttttaaccattcactgattctcgtaggcgtggatgagaactgaggctgcctctttcttccattctctttcgttccttttccttctctttataacacccccccccaattctttcatttccccagttgcttaggaagacagacagatgcacactaaatttataatctagtttgtgcacacagttgatggaggaaacacagccttccaagttacatccttttttaaactgaagttttaagtatgtatcttaactgactggggagattgaattgtcacttatgctaatgtgatatattggaatttgggttagtgatcaagattggcatcattaaagagaaacaagtcatagtttttcacatctgagaagtcacctgcttttcatggctgcataaacagtgattacaaactcagcacctgagagtctgcctaggagtctggatctgcttccgctgggtccttcctcagggaacacccaggctaactgaagttcatctgcctgttgcctctcctcctaatgctctgcttgtcttagaggcttactggttggcagaattagggttgtagggctgatgtctctgttttcttgcaagccacaagtcagggatccttttgagttcctcagaggcctgtctgtgttcttgccacatggccctcccaccagtatagatctgaatcaaaggttagtgatagaaacatctgtatatgtgcactgggatggcaaatggcactgtcacatgtgcttgtttgctgctgttccctgagtactttcagtcaactttactgtggcataatttatatgaaatgagctcatctatttaaaatgtatgatttgaagagatttgagatatgtatatgtcatgaaaacactgccacaagggaagacatttcatcattctttaaagatggttccttgtccagtctggtagtccctcctgaaataacttcatcttctgacactcattgctgctttctctagcctagttttactttggtattttaaatagcatgatgaaaaaagaggagatggtgaaaggttattcctccatttttttgtttttgtttttgttttttttttaaatactattttatcttactttacgcctatgggtgtttgtttgtgtggaactctgtgcatcataggcatgattggtgccagaagagggtgtcacatcccctggactgttacagtagttgtgagccaccatgtgggtgctgggagttaaaccagggtcttctggaagagtgaccagtgcttttgactgctgaaccatctctccaggtccgatacatattactcttaacttacgctttcctcagattccttcttggcaaaataatcctttatcagataatgctttatggttttgggatgtggtaaaatgattcttgtattttctcaagatagttgcagtatgctcttgttctctgttagatctccaaagctagatagatttaaataggtagcttcctcatctactgttccagttactgtctcttaaggcccaaaccaggtaatttcccattagaaattctagtagtagtgaatcttttcactggttactggaaacccaacccttctagagaagtcagagtatactcacctcagactttctgttgttacagttcatgtcacagtgtgaatttagaagacatgaacagtttcccttgtagtcacttactgtcaccccctccccctgctccttgtataatgcagtgttgtgcagtgcaggaaggttttaggaagccttgcttcttgcagcagaagaaaaagagagctcagaggagactcagtgagaccctctaaaagctgtcaactgtcaacgagacagtgttgatcaatgctgtatacctgaatgcaaagtttctactaatggtattgaaacagatagtagtaattttagtgtatttttaaaatgtgtttactatatatttgtataatatatccaaatattattacttctgcatttaagctaagaaaacttattgaggtagtgttattttaatattaatctttgagttcctgtgcatattttatcctttacacagtagtgtcacttccttgcttccctcccttccttctctttttcctccctctttggcatgtagcttagattggccttgagtcccctgggtaacaaaagccggtttcagacgcttcattcttcatccttttgccctgttggggccgtagacagttgcaccgtcccaggggcccagaggtgagtctggtcaggttgtaatagcaggtgctcgctatctactggcccctccactagacagtgcttatgctccagataagcaactctctcttttatgtaacattacatgactccctaagaaaccacctttgagtaaatgtttgattatcttgtaggttagggtggtagctcagaaggtttctgtggttaattttgtgaaggtaaatatactgtaatacatgtttattatacagatgatgacattattgaagaggtggtggggtctgggaagaaacttgcttcctcagtcaagagttaactagtggtaaacttgggctgagttttcttcttttaagtgtttcactttaaatcaaagagaaatgaattcctttttgatcttactctacatattaagacatttattgttagcatctgttttactgctagatctttaaagttttgtatttttttctcagggttgtactcactacaggtctgcttgtggtattctcacaatagtctgaatgtttcctgaatagaattagaaatctgttttaatttactaaaattaaaacagtcttatagctcaaggtggcctggaatttactatgtaggtgaagatggtcttcgactccttatccttgtgtctttaaggagtgctgccctaactgcttaagggaagctatctcctgtctcacccatacttttcaggcacagaaaaagaggtaaagtagaataaatgggaaaatggaagccaagagttctcttttactttgtttatagaatcagaagaccatgtgagggtctgttgtgccctcccaggtgttcctgtcctgtctctcagcgtaaccttgtctgtcctggaactacagtgtatgccaggctactgtggaactcagagatctacttgcctccctcctgagtgcagggattaaaggtgtgcaccaccattcgtagctttacctggcatagtgcgtctgtttaaatgaaggaaagaggacttgttttttcactgctgtgacagaatactcaagaaaaccaaggaagacaagatttattttggctcatcatttaagaatggatacaagtttgtcatggtgtagaaggcatggcagcaggaacttggggtgtctggttgtgccatgtaacctagagggctttgcattttattgagcagggggtcctgcatatctgggcctgtctggctgcttcatagggtagcaggcacactgttgctgactactgccacctttgcagagctgtagaagggttgtgttaactcactgagtgtgtcctagtttccttctacctcttgcactgttaggatgatgttccacattgtaagtaatttctgcctcagaaagcagctccagcagtcactaaaggaggactcccagttgctatgagaaccttaggcaacgactatgtaacagaatgtctcaccctaaaaaactaaaaaagaagatttattacttatttgtgtgcctgtgaatgccaggagagggtatccagtctcctggaactggaatggttgtgagccacctgatgtgagtgttgggatttgaactcaggtcctgtggaagagcacaagggctctcaacagcggagctatcttctcagcctcagcagttcttttgatgccacttttccttctgctacccaggaagcagtgattgctcaagttcacctcaaactgtgtaaaacgcattacctgtttattgtctctgtgcgtgtgttttctctgggccagagtctaactccgaaccactcacgagtcacatttatttgctgctgtattagcaacttcctattgctgtgataaaacaccacagcaactgactgaaaagtgtttgtttgggctcagagttccagagcatagggtttccttcacagtcatggtggggcagcatggcagcaggcaggcgtggctgctgcagttggagcttttataacttgggccacaagcatgagacagaaagtggcctgaggatggtgtgagttttttaagccccagaatcccgcccccagagacataacttcttttaacaaagccacaactcacttccccaaacagtaccagcaactgggaccaggtatctaaaaagctgagaatatgggggacatttcattcgaatcaccagtgtccttaattttcttcagtctgaaacatttctaaaattcttgccttttattattactttggaaaagtactggttgtgttgaatacctccagtttttgtttaatgttttggtgacaattgttgggtaagagtactacagaaatgctgttgtgactcttactggatggcacacagtgtcagtatgcccattcatctggtgagaccagcactggtaagctgatgttaggctttgtaccagttgcatgctaaccttgcaattaagtgtcctatggggaaggtatcttgtgattatgttaattaccttattttcatcatgcttttagtttctaatttcaaattctggtgataatttgtatctgaatgaatattattactttggtgtttggccagtgttattttctgtttcttttctttcttctgtaaggaagagctatctcctctgaggtatgtactttaagtgtatcaaaatagactcatgaatattttctctatcctgtagactcagattttattctctggatttcatctatttctttctcttcctttggtgctttggtcatctgaggtttggtcctcaggactctctcaatctttcatcttgtctcaatcactttttgaggacagattatagcagcagcatcagttgtcccaggcttatcttgtccctttttttcttccctagccttcaatttggccatctcaccaaggagttatttgtttatttttactgtgggttgagttttttccatttttttctccattagcatctttaaaaacgtttgtgttagaatgactttggatggcacagacatatgaccctgctgtaggaatactagtgcatagccagtggttgtcttgtgatagactttgtctcccagcaggttcactcatttagtcatggatgagttttacaccttaacattgctcacatgttactccatggaaacaactgatatttttctttggttttgttctgtgtttggtaataacaatttaaaaagctcttctaaaagtttccttctaatatcacatagtatagaaatgtttatagctctttatttatctttatttatttatttatttatttatgatgttatgatagtttcagtttttcaataaagtttaaactgcttctacgttcttctagttcacttccatgtcagagatggcttactctctagaatacatttgtctgtctgtctgtctgtctgtctgtctgtctgcttgcctgcctgcctttgtgtgtggtttgggatggtgtgagctatatttatatgatgcatgcacatgagtatgcaggtgtatatgcctgtgcacgggtatgtggaagttggaggaagtctttgagtgcctccctttttggctctttgcggtattatgttgacccaaggtctctccgtaagccaaaagtttggcattccagctaggctgactggctagtaagctcctgcttcctccttccagtggtctagctgaggtcctcatgtttgcacagcaagtgttctttctgtgagccggctcccaagtcctgcagtgcattttacatcaggcttcacagtgcctacagtagatttcatttttcgtcctgctgctttggtttttaaagcaatgtttgttctcaatttatttcctatctctgttgtaaactgcagcttgaaatttgcaacgcctggaaagttggaaacaaggaaaacatcatggagcatggtttataagctacagcttcccagacattgtgcttttagctaagcaaggcattaactagtgcccccttgtacttgggctgtgtcagtttgcttcacaaaattcatccatacttcccattaatttatcccagttttatttctaagtattttcccatggtcagccatggcagagtcaacctcaactccgaatgggcacaaacacacagatgttcctggctgcagccagagagtagtgtacttgctcacttctccaggcgttcttgatgtgttctgaagctggaggagtactgatgcatttcctagtgactgcttcagaccgcactgggaacaggcgtgccagctcatgctctctgtctgttgaaagcagctcttaagtttctgcatcagtctgctgtttttcagctgtttcttgtcattttttttgcagctgatttgcttaataattctaattagcttttgtttatgggtttatgttaaaaagtataaattttgattgttgattgtgttatggaaattaaatttcatcattagtgtctatacttgggtagctagctcacactaaacttgacttttggatgcttcagttcagtgtgttcatatgcagtgagatatgagaaagtaaacactttttttgttgttgttgaatattgaaacacattttgtttagtttagagttaatggtgggttaatttctacgaaattcttttactattcatccagtgaggagagagcacaagatctttaaatgacttgtattttgttttcattcccacagactgggtaggatcactattgtattgtattggcacatacacgctggacagctggtgaactgtatccccaatctttcataggctgtctcagaaaactgagcacgagcattttcaatgtgtgtcatacagggactaaagctacaagagaaatattgctttcttcttagatatttctagcatagaacacttggtttgataaacctaatttttagtttgctattttttgacatatatgaaaaattagtaaggtccatgtcattatcagtagttattacttggttattaccattttggtttttttgacacagggtctgtgtgtagctggtgatggcatccaactcacaatcctgctttagcttgttgagtgctggaatcacaatcctgtgtcaccatgaagggcttattttaattttttaaaaagcagcatcttgacagtatttcttttatttggaagttctttcagaacttgtctgaacttgtctgaagttgtgattggcagtgcccttgtcatttgactcatttaaaactgaaggaagtaatttttcaaaatttgagtttattgatatttttacaaaacatcttcagactagccgagagtggccttgactttgggccctcttgccaccacttccctagcgcagggattgtaggcttgctccagcatacctgcttatgcgatgctggatcaagcccaggtccctgtgtatgctcagcaagcattctaccaattgagtggcatctgccagctgagccgtattgccacttgagctgcttccttttgtatccactccttagaagagtataattttaaattgtgtataaaagataataaaataattcagatagaaatcaaaaggcattttaatttagtaaccatctaagatttatgtaatttttttttccattttttattaggtatttagctcatttacatttccaatgctatactaaaagtcccccatacccacccacccccactcccctacccacccactccccctttttggccctggcgttcccctgtactggggcatacaaagtttacgtgtccaatgggcctctctttccagtgatggccgattaggccatcttttgttacatatgcagctagagtcaagagatccggggtactggttagttcataatgttgttccacctatagggttgcagatccctttagctccttgggttctttttctagctcctccattgggagccctgtgatccatccattagctgactgtgagcatccacttctgtgtagatttatgtaatttatataggcatcctcacaacctgtattgtaaaaaattttcagtaaatacattaaactttatattgaaagttgaaaaacattcacttcaactgaaatagttagtggatactgacaagaccagtactatttttatgtataattttgaaaaggcttatgtcataggaatattaagtaccagggaactttttaatcaagagccccgggaagagctgctcagtattaaaattttctttgtagcatgcttcactgccacgaaggattaaaagaaatgataattcaaacattgttcatgggtaaaaatctatgtaataagctggcaggatggcaggaaaggtgattgccaccaagcctcatgacctgagtttgagtctgatgacccagccttgtccctgtcactcatctgatagagaactcacttagatgagttttcctctcacctttatacacgtgctgtggtttgtgtttgtgtgtgcatgtacacacagacagacagacagacacacacatacattttagaaaagacgataaatcaggaacaatctcatttaatagctcttttcacaatatatatgtatatgtataaaggtctcaagtttagaacaaatatttaagttacacattaaattctattgtgggaagattgttaatagatctcaaatgtttaatattttgatttttgagtagattatcatctgactgtttctacccaccagctcttaataatgacacggagactatttatttatttatttatttatttatttatttatgaaagcttaggccttagcttgttactttctaactagctcatataatttaattaacctgtttattctaatctacatttgccatgtggctccttacctctcactaagtctgatttcctcagtgtctggctagtgactccctcacttctcttttcttcccatagctactatctctgcctggaagttcccctttcctctcctgtctcagctataggctgtcagctctttattaaaccaattggaaggtggcggagaagaatgtttacaaaatactcaggcaggtgatgatcgaataaaaataccaatatgagaattcagtacatttgaataatacaaagacaacctttacacaatgcacagaaagattatcccaatagtagatacactttttttttttttttttaaagtttgcgtcttctgtaatgtacttttaaagctagaggtagatgtgtctggagtagttaccacaccagactttctttctcttctttgattaagtccagcagcattaagttcttcatgtctgatgaccacaggaataaggagtatttctgctggcatttatcccgctgactgttaggatttgcacaatacatggtgatgcccagagaattgctttaaagtcagaggaataagcccatgtaaaaaaggaggagacacttgagcaatcctgtaagcttttcttttgagagaattcgtccagtagtttgtttgtgtaatgaattctgtgacaataattttaggaccactggtcctgaagagagttttgtgattaagttgaagaaacaccatatactgtaactatagcctattaagattcatagtgcacattaatatgctaagggttctgagccatctcttgaaaaagaagtttaccaaagcttgcttatcctgtatttcttaagtgtcttggacaatggaattctttcattcaagcttctctgtcctgtgataaatgctgccctctagaggcataaattcaccggctcttcaatttgagcaccttaatttaatacttagtgagtagtaagtggctcttaacaccaagtgcacaagagtggaatagcctagtagaaaaagatggtctgatttaattttaagaaaatgtatgctctaaattggctttattatgtttactacaatcatctttgtaatcagttctctctttggtggcacttgggatgggatctagggctttgcatgtgctctgtggaccttctactattcactgtgctggaataacattcttttaatagaagaaattgttaccgtttaatttttattacattttaattttgggaacacatacatagagttcagagaataacttccaagaatcagttcttacctcctcctggggatcaaattcaggtaatcaggcttttcatcaagcaccttgacctgctgaactgtcttgtggctcaaatataggggattgttgacacataagtcttcatgaacctactatgcagttcagattggccttaaactcctagtgaacctcctgcctcatcccaccacacccatctttgtctactttattgaagacctgatacaatcttgcttaagagagtgagcagttgttcccacagtccttatgtttccagacctctaactggagccaagattatgtcctacaacaccctcatctacttagattatgtcctacaacaccctcgtctacttagattatgtcctacaacaccctcatctacttagattatgtcctacaacaccctcatctacttagattatgtcctacaacccctcatctacttagattatgtcctacaacccctcatctacttagattatgtcctacaacccctcatctacttagattatgtcctacaacaccctcatctacttagattatatcctacaacaccctcatctacttagattatgtcctacaacccctcatctacttagattatgtcctacaacaccctcgtctacttagattatatcctacaaccccctcatctacttagattatgtcctacaacccctcatctacttagattatatcctacaaccccctcatctacttagattatgtcctacaacccctcatctacttagattatgtcctacaaccccctcatctacttagattatatcctacaaccccctcatctacttagattatgtcctacaaccctctcatctacttagattatatcctacaaccccctcatctacttagattatgtcctacaaccctctcatctacttagattatatcctacaacaccctcatctacttagattatgtcctacaacccctcatctacttagattatgtcctacaaccccctcatctacttagattatgtcctacaaccccctcatctacttagattatgtcctacaacccctcatctacttagattatgtcctacaacccctcatctacttagattatgtcctacaacccctcatctacttagattatgtcctacaacccccttatctacttagattatgtcctacaaccccctcatctacttagattatgtcctacaaccccctcatctacttagattatgtcctacaaccctctcatctacttagattatatcctacaaccccctcatctacttagattatgtcctacaacccccttatctacttagattatgtcctacaacccctcatctacttagattatgtcctacaaccccctcatctacttagattatgtcctacaaccccctcatctacttagattgggaagaagaggattgtgtagtgtgttttaagacttgcaaaaactttatacgtatgtatagaacttgtttcaaaacatgggaggggaggaaaaaaagaggggtgaaaccacgtacgaatagacaacatgcttttgtatttttgctgaatgagttaatgtgatttacttacagagattctagtttcagtgttttagttcaaatagttctaattatttgcccttttgattttggtattgctgtgcactaactgaaactcatggtacagtttaatggaagtgaaaaagttaaaacaaaacaggattgatgaagtccatttatcatgttgaccattcagctatcttcctgactctccagaaaattccacttatgatcttgaacatactgcatttacctaggtgggaataaaaacctttgtggcactgtgactgctgctgtgggtgtcttttccctctttggaggaagggctgataggattctagatgctctcagttccgtaagaaagataggcatcatttcataaggaaacatgctaagtgtgatggtcaaagaaacactgcataaattctccttggctatgactgtaaaatggtagaattcctagatctcaaataggagggcagcccattagaatctgttaagttctgtcccatgccccaccctcctaatgctggggattgaacctaggtctttgagcacgatagacaagtagccctgctacttttttagattgtatttcaaggtagtatctaagttacacagggtttccttgagtttgtgacccttttcttggcctctgggtatctgctagagggatgactaagttccttcttatatgcttatgtgctaatacattttccctcattcttttcattcctactccccttttgtaatttagtctgtaggttatagaaaagccaggcaacggtcatcacctagaatttctgctattggagctgctgtagcagctggtgggactggttttttaacgtttttgtgaggagcagaagatttatacaagtcttagagtttgtcatcttgagaattttgtttagagtttattgttaaaaataattgtgtgtgttgggtcatgtttttgattgtttatttcagattgcttattggtattgggatactatcatactgggctataattcacagaatcctttgcaataagatacaggcatgtggtctaggcactgtgcattacatggatgcataagatacaaatcccaaagagaagtgcaagctaacagctcttgattggaatggctgcttcatgtagtttgaggcttgagaccctgagcttgtggggcagcagtgcggaggggccagtgaccttgtcctgctccctgggcttttgtggtactagagtgctgttatggggatcatttgatgactttgctgtttttctgctgtacagcctgtgagtggatgctttctctgagagttacttacatgttttaacaagttcttttttgcaacttagatcctggttttatagatttttgtaaatattttatttttctgagacttaaaaattgccaagcacatctttcacagttttccttccccagaggtttacattttgacaccatttttcttgcttggtgtgtgttttgcacctttggattgttttgatgtttgtccttcacctcctcagcttcagttccactgtagttaggaaacctttcctccagctggcactttttttccctgaatgccagccgaagttgtctaggtgtgaagtgttgctttttctttctgtatgcatcttagtggcacactagtgataagctcccagtgagctgctaacaggcttttctgtctcttgcattaccaggcagaatgcactgtttagattcaaggtcacctgaaaaatcacagtgaagccaggccgagtgagctgagataacaagctagctacatttgattcaccttagcaccctgtccaggatgtccaatttccttcttccttacagagactcagatctgctctcattaggaggtcatggcctcgccatctgaattacttactgtctttgtaatggtttgtgatttttgccttgacaaaaacctgtttgtctccaagttcctttcattgtaacttttggatttggtattcatcctcagcaagcttactattttaaataccattataactctataaataatgaagtaaatcagacacactgtagaaatccagaaggtactaaacatacctcttgtgcccaagtcagggttaaggagccattaacaacctttgtcctcctttgcatccctcatctgatgctctgaactgatgatcctcctccagtctcttcctgacctgtaggggctcttcttcctttcaagttctttccttctcagctcgtcttcctttgttactaggtacccggtggcctaaggtcctttggccattccactcttgtttttctatgtctgagatgttcataaaatgacctgaacagttttacatttcattgctttatagatgcattccaattgacataaaaaacttagttcatttcttactgcctctgttacctttgcatgttgagtaagagtcagttttgctgagagataactaggcttttttgtctttggcaagtggtgcgtaatgctcgcacatggtatcgtaatcaagtgtgtgagttgatttgcgataggcaccagaaagtaggggctcagaggatctaggttcaggtattgctttggggtccaaaagttacagcttgatacctcccaccctgtttcccttaatttttatctagtggcctccagttggcctgtgtgagtttgatgtaaaatttatttattactttaatttctagtgaagttgagcatctttcatcactctgatatcctgttctttgaatcttttcttcgtatgctctacatttactttttgtattttccctttatattgatttctggcactttttatagtttgtgcattagtgttttgagaactaagcattacaattacttttctaatctttgttacactcagttctgttagtgttttagtttgttgcttgtatctatagttgatgttgagtgtcagcctgatagattgagccaagcctaggacctcagtaacaagcacttctgggtgtttctttccagagtggattaactgagggcagagctgccctgactgtggactgtaccattcagagactgagaggcagataaagtgaatgtgggaacgaaaatcaggtgtacacagctgtaatcccagtactccggaggtcaggagttaaaggtgcttttggcctacacagaagagccctgttttcaaattttggggattgagaatgtactccagtggtagagtgttagcatagcatgtgcagccttgggctagatctctagagttgcacttcacccaactccagcaagaagggggcggaaacctgagggtggaggtactgtgggaagcctgttggcatagacagacatcctgtctttctgtttcctgaccactgatatgacaccatgataagagtttctctcctgtgtcacgtcctcctgccccaagtcagagggaatccctctttaaaatgatttctgccatgtatttttagtggtggaaaaagcctgttaagaccgtgagttttatttgtgttgtttctattctctgggctgatttggtttgtcttccttcatgtcagaatcacactgttaaatgagaacagttctaatgtgtctttgaaatttaataaagtgaaccttctctgaaactttcaggtcagcttatcaagtttcaaacatccttccaaaataatctaatatcaagcaatttctaacaactcttggaattatgattggaacctcactgaatttacagttttcagtgcggtaacagtcatctgttcctttatgtacttttaaatgtcttctggtacagttgatggctttcatagacataatctttagatttactcttgttactgtagctctggctggcccagaacttgctgtatagactaggctggcctcaagctcagacatccacttgcctttgcctccaaagtgctggaattataggtgttgccaccatgccccatttctagttccacctccccatttttgtcccccccaaaacttttaattcaaaaaataggttctttcgggtatcctctgttgataagcctataattagcaaatgctgcattaatgctgcccaagtctgttccttttaatttttcttgtactaacaacattgaagcccttccttgttcttggtcatttttatgctagtgtgtggaagagaggtagatttgatttttaaggaccaaaaagtcttaactatagtccttataaaggttaaatatctagttccaacaagcaacctcttatagggtagacactttgttcctttgtagtcttgttagttgtactgacatgtgaagatcactttcatgtcagctgaaagattaatttataagaatggctttgatatatatatatgtgtgtatatatatatatatatatatatatatatatatatatatatatatatacacgcacacacacacacacacatatcccttcttaatggggcttctgagggaggctattcagaactggaaatgtactcagtatttaatatatttctgtaactgaaactactgtaactgaaactactctgtttggatgcttacggtaacctgaatccttcttgatgtgtgagttgtcttatactttgttataacatggcttaagagaataacttgataaaccactttatacttcatgctgtttccttcatcagtagaagtatcatcataccttaccatctaaatggctactagaattaagtaattcccagaaggcattcagaagcgtctgatccctgggattggccgtctatgggctgccacctactgcctttctggaaatttctttaaattctagaaagatactagtacaactgcatttccagccctatagtaactcttcttggcttagtacttgcatggatgactagctagtgttctgggttttactttctgtgatttttttttttcccctcctctgcagcctattctggttggcctggaaattatgttaatgattgttactcctaggggctggagagatggctcagaggttaagagttcaatttccagcaaccacatggtggctcacaaccatctgtaatgggatctgatgcctttttctggagtctctgaagacagctacagtgtacttgtcatatatataaaaaataagatttaagattttatttatttaaaaaaaaagattgttacacctagttaatcactgtgtctttcaaggaagccaggttagttgctgccactgtagctactgtactttctgtgcgttagcttgctctgtttccctttcctattttaacttgttttctttacttttatactacttaaatgctgcagtagaaagccaccccaatgggtacacatatacacatgtgtatatatgtacatacatgtgtgtgtgtatgtatgtatgcatctaaactttagattataaatgtgtacttgggtatatttttgtaagtttaagattatagaaactgctataaattcatgggcatcaacaagtagatgttggcatagcttggctgcctgatgcagtgcttcttctgttgactgcttcccttggccccagtacctaccattcagtcatttgggctctgtaggctcccacaatgtctctttctacagaaggttttgttctcactttctagggaagacaggactccgtggtgttccttagtgtcttccttcttactgggataggctcccttcctctccacagccgggcatgctctgctgctctgcgtcattcttcactgttaagccaaagttaaagttattttcacttagctttttccttcaatttagttattaaaaaaattaaatttgcttgattcttggttctagactattctctcctgatttttttttttctctttccaggcattcgcaatatcatgactccatgcagctatcttagaagtagatcagacaaaacaagtctaaatctgagttagcagtcctcactcacataatttcacctttagcctactgtctcagcctattctcatggggcttcactgtctgtcttgtctgtctctctctctttcttataactattctggccttatgctctacatgtactatatttttaatgtagaaaaagatactcatgtcagtcattagggaggtgaaagtttagcctgcaagatagccatcaaaaaatagccagaagggctaaaattccaaggactcacaacagagatttccttgggaagataccccaggacccttttagatgacacctctggcagcctcttacaaattgaaacatgtactactactggacattgtcctcctatgtattccaaagagaaatgtaaacactcattcataggaagctctttgaaaggaaggcgtactagatttattcagaatatctgactactggaaacaacccaaatatctttcagtagctaatggctggagtttcctgcagtggaatgctactcattaataaaaaggaactcccagtgtaaaaagagacatagatgaatagcacagacacactcagcagaagaacctggatacagaattctataatttctcttatattaagttttagataagacagagctactctacgataaaaagtaaaaattatgaccactgactgcctttgagtttaactatgctagtggtggatagttgactggatatgtttgctaaagtgatacaaatgttcagtgtctgactggtatatgtgtgcgcgtgcatgcgttacagggtatatttatttgttggacagctaagatatgtgtgtattttctggtatggatgatttatctagtgattataaaaatagttgaatgactgggagtgggcggcaaagatgcttgaaatttccacttaagatttttgaaggtcaaatgttataaagatataaacaacaaatacccgtgcatgcatttatattcttcaataaaaataaccttattcatatgataaattcctttctcttaggcttctatactgactccttactcttcagtccctttttgctgttttttttttttttttttttttttttgaggcagagttttactgtgtagcccaggctggccttgaagttgaagtccttctgctttaagcgctagaatgcaggtatgttccaacctgcccaccctgttctctgctttaaacatctgtatgtttgttgaaaggtcattgccatgggggaaagctgctcttgatttagaacattcactagggtgtgccctttcatagtttatcacagttatggttggaattaaagtgacccttgcaggttcatgttttgaaatcctgttcccaagctggcgctgtatggcaaatgggcctgatcggtggaggttggtcactaggagtggtcttgaaggttacagcccggactctgcttctgggactttgtacttccttgtctgttgtaatgtgaggaatctctggcacaggcttctgccgacaggaactcctctccacccttcctgcagctgtgcatggggatctccacccttcctgcagcagtgcatgcagaccatttaaattgtgcgctcagtaggtccttccttgtgtagtttctgtcaggtgttttgtcagggcgagggacgagacaagtggttaatacaattacatgctctctccagaagacttgagtttgcagaggcagagctgttctagtattgctactactcaggctttttacatcctagtgaacagttcttgagaaaagcaagtaaggaacacatctctctgtatgtgtgtgtgtaagtatatgtgtacatgtgtgtgcatgtatgtatgtatatgactacactttatttcagctcctgattttagagacttgtgttgattatgatgaggagggtatagcacagcaaagcatgaacatcacaacagccttggaaggacatagaaggagatccagtggggcaagggcaagtgatagccccaggttcacactccagtgattgatttattccagtagacccgcctcctactcatcaccacctctcagcaatacagtcacgagatgaatccatcaggagattcatacattgattaggtcagagccctcagaatccagacacttctgaaaatctgtcagcagacaaccaagcccctaatactttagactgggatgggaggggagatgaggcattacatactcaaactacagtgcattttcatgctggggtatgtatatataactctcttcccccacaaaccctatcttgcctatagctctcatttgcttttgtcacttaacaaaacaccacagacatctgctttatgattaccttcattcaaaacacagttcaggctggagttggctcaatggttccgagcgagcgcttgctgctcttccagtagacatgactttggttcctagcactcacatcaggcagctcactgcagtctgtaactgcagttccaagggatacaatgttctcttctagcctttgtgggcacctgcatagaactgcatgtaacatacacacttgcacataattagaaaaacaaagcagttctgtcgcttgattttaccagatattcactttttatctatgaaatgtgaatactaacagtttctgtcttttaggacttggagagcataatattattactatttcttaatgaggcatgcaaagttcccaattttaagtgttcagcatgtattgaagaaatgttggcagaactagccattttgttcctttaaatttttttctcatatactacattttgattgtatcctttcctttgagatttttcccacctcacttcatgctctcactttctctctttctctctctcagaaagtaaaagcaaatggtcaaataaaacccagtaagataaaaaaagtaacaaagcaaagaaaaagcataggattgccccaaatccaaaccaaacccctaaaccagcctgtaaagtctgttttgtgttggccaactactcctgggcttggttatatatcaattgtaactatcttcttggttaagggtggggtcacttccccttctctttgctccagtgttgtctgggtccaacttcttgtgagcattgtgcatgctgttaagtctctgagctcatatgttttatcagccttgttattttggaagatgctgtttccttggagtcacccactgcctctggctcttatgatctttccatattcttttacacattgatccttaagcctgatttccaagccgtctgtgttgactgtgggttcctggtagagagctggtttagggatcagttgggaagctggtccctaaaaggggttactgaatgggataacaaaaagaacgaggattttaaacatttttaatgtagacagtcaggtggcagttatattttgcaacaatcacttttatgtaattatttttataatcattcttcccagcagaatgctttaccagcagaataattcctcacaccttccccacagccatttgtgacttctgttgtgcagtctctgtgaacttgagggttccaggtagcacatatacatgcatttataccgtggttgtctctgtatgactggcttatagccgtgtggcataatgctttcaaggttaatccatgtcttaccatgtgttgtcacactgatttacaacttccctccttgtgattggagaagacactttcccagtggatgtgaaaacatgtccatagagttttcccaagaaaattgataataaatttattaaaaatagctaacgagtagacatgtaccagtaccatggacattcatagtaagattataggaaggtaaccatgatcaaggttatatgttctgagtgctgggctggcaagatggtttagcaggtgagaatacctgctggcaagcctggtgacgtgactcctgaaacccataaagtgttagaacagaatcagctcctgagagttgttctctgacctgcacatgcttgccattgtgtgtcctctaacaataaaatataataaaataaaacactcagtgttttctattgaatttagaagtagcagctttgtatcttacagttctcaggaacttgaaggattgaagatacctgatattgtagtcatttcatgtattaaacaagtagtcatattgatcatgaattgcatttaaaaatccagccttcatgttcttcacgtaccagtggtgcctcatttccccaggtctttatgccttctgaaggcattggaaatagagcctttagggctggagagatggcccactagttacagagcgtacgtttgctcttgcagagggtcggagttctactgtagcacctacattgaggggctcacaacttcttgtaactccagctctaggtgcatttcataacataactctggcctctcagggtgcctgcacttgtgtatgttgcatttgtgcacacacaaccacatacataaatacacacaaaccttttaaaatcataaaagaaaaggagaggttttggtagttcagatagttgagaggtcttttgggggtgggggtgctggggatggttcacagcagtgacctttctgaataacaaaccatagtgcttcttacaaccatgtagttaattgcccattgtctgaaaccacgtccagattctagagtaactagacacttcttcatgaaccaacagctattttccaatataataattcctaaagctagttccctatatggatggttacacttagtcttactaatgtattaaaagtctggttgtggcatttgattttatatattttatatttactttgtatatatgctgttcacataacttacagtgtcctttctttagtctcttctatttgttcaagcttaattatacctgttaaatcatatttagtagttaacattatcatattgtaatttactttagttttcctaggtgtacatcttattctccagaataaattgcttgaggtcgaagaggagttagtttggcagtattttctgtgctgtgctagctgaagctgacagtgctgattggaagtgatcagttatgcttttgattttgtgttgtgttgtttctgagatatttagtaggtagcatggattttatactttggtcttatgggtaaccacaacagaataaagtatatattagttaaattctcaacatctcttataattggatgtattgtagagagaagatttgagtttagtgaggttttaaagtaaatgccttaaagaaaacctcaagttcatatgataaaacagagaaacatttcaaaattgaagtccctgaagcttctgatgtaagttgaacaaatgttgcttccgcagccacagcaatagaggagtagctttattggctgaggcacattagtagctttgtcgatgagaagagctaagggtccttatttcaattcatggttatgctagagtagtgaagtgagagctgttgacctgctcttaagcagagactagaaagtttttacaaatagattaaagtacatagagaggtgtgaggagctaggcatgcctttaacattaggaatttcatccttttttgtccagtcttaatacagttgaccctacaacacttactaattgattctgaaaacaacagctttaatgaggtggtttaatagagttaaggtaatagttgagttgttggtacatctctgagtgctattggatacaaagagtaatatgagatacaggatttattaaactggagacaatgtaatgccactaggatggcacatctccctatttgttttatgagtccgtgggcatgactatgtggaggtcaggggacaatacttggagaagctggctctgtgtttccatcatgcagacccaggggattgaactcaggttgtcaggcttggcagcaagcatcttcatctacactgagccgcccttttggtccttgaactgtaacttacctgttaggttttttttttttttttggttttctttctttttttttttttttttttaaattttactttcataattttcaattgtgcgtattatggatgtttttctacattgtcaggttgttattttttcctcatttctttttattctctacttttcatatttttcagttctctcatgtttttattcttaactcatgatatttttcttaattttactttcctggagaaggctatcctagtagaggttataattgccatggtgaagtactataactaagaacaatttggagaggaaagggtttatttggcttttgtgttctgaatcaaagtccactgatggaagcccaggcaggaactcaaataggatagaaactgtggagacaggagctgatgtagaggctttagagggatgttctttgctggcttgcttcccctggcttgctcagtctgctttcttatagaacttaggaccaccagcctagagctggtcccacccactgtgggatgggccctcctctattaatcagaaagccctgtcagtgcttgccaacagcaagccatcttctggaggcattttctctgttgaggctccctcctcttagatgattacagctgtgtcaagttggcctaaaactgacaggacgagaatcagctcctaattcgtttcttgctttgtcttacccattttttatttgtcttttgtatttttagttaaaggtggaaaaaaaaaaacttttaaatgtttacttcagtcattccagttgatcttactttattgtgttttctgtggcttttgggttttaaacttagctgaagtttgacttggattatcttgagtacttctgcttctgttccttagagtcctacaagtgggagctgggaagaaggagggctcagtggttaagagcacatactgcttttctggagaccagagtgaggtttccaggatccatatcaggctcacaacagcctggaactccattcccagaagacccaacactcttttggcttccaaggataacccctgcccacaatgtgcatgtatagataagtaaaaaacccaccaatttttgagtaccatttaaatgtgatatactaaaactttttaaaaatagaaaatagaattgttttaagttttttttttaatttagaagaaattttaattaaaaaagtacatatgtttatggagttccatgagatgttatgatacatatatgcactgtgtgatgtttatggtaaggttaatgcatcaacctctataaacagttaccatttctttgtagtgaaaaactcaaaatctagcttttttttttttttttttaataggttataatgttgttctgtgtaatgattaggctactgtgcagagcactggaacttctagctctgagctagagcctgggaaagtgctggcttaggtatcagtgtttgctggttaccatgcaggaagaatctcatgaacttactgttgaacagtgtgtagctgcttaggtaactgtgattttctgctttagtggacattccatgcttcttccatccttacagtctttctgatctctctgtgttctcagaaagcataaaactgggtacttactccttaacaaacccataatctgcttctgtgggctctttagataatgcatgatctgttttttctcttgcctttggtcattgtagactcttaacttcaaacttcaaagtagatacctctccccctcccctcccctccccatcctcccctcccctccccatcctcccttccccatcctcccctcccttccccatcctcccctccccatccttcctcccctcccctcccctcccctcccctcccctcccctcccctcccctcccctcccctgcctgccttccttcttccttcctgaaggatagatctctttggtgtaagagttcagtcaggatgacaaatctcttattatgaaggagtttcccctttattgttataagatacctctccaattaacatttacattaaaggaggctgatatttctcttaataattgagagacaactttaaagcagtatggttcatgcttggagggggaatgcagtctgacttaggaattgatgcagtggaagtgttcctgtggctttgctctaagtgttggcatctgtattgtgtggctgtcttgtcttctcacagaaccctcataactcctggctgattgagctttctctacaggtcaaattcttatattcttgtaacattttggtgagtctacctctaatgaatgctggccttaggagaaacttaagtttctcagcatctggggagtgctgagagagcttatctttaaggactgggttttcaaacaagttggtttgtaaatggttaaaggtgcttgctgccaagtctgccaatctgagttcaatctatatgacctacatggtggactaagagaatatacactgggaattgttctctgatctccatgtgtatctgtggaacttgtgtgcatttgtatacatgtacgtgcacacattaaacaaaatgttaaaagaaagcaaaacgtgtcacagtccatggcaatctgctgttgtgaattacaaagtgtaggtggctttttttctcaaatgtgttaaaaacttcaaattgaatcaggatgctaatttagtttagagagaaatagaaattgtttacattaagagacaaatggaaaagtaattaaaaagaaatgagactgcttatattttaaataaagaagatactattgtcaatattatgtagaattttagggatatcagaatcttcttttgtaaagcattcttcagcaggtccaaaagactttcttaaaaaagacattggttagaaaaaattcagtttgtaaaacaataggactgggtagttttctttaaaggttttacaggtccctggcacgaccaccatctgttcactgctaattctttatatgtgtgctggattatatatttgtaattttgtttgtgtgcatatgtgcatgccacagtgcatgtttgtgtggagctcagaagacaatgttcaggagttggttctaactttctactgtggattctggggatcaaataagccaggctagtaaagttctcacttgctaagctatcttaatggcccatatgtttgtggattttacctgaacctgttctaaggtagacctgggggaagggtgtgggggtagggaggagataagatatggggcataaataattaattaaaaaatgtattagcaaaatttggtgttaagtggtagagctgtagagtgcttgccttgcatacatggcatcctaggttcactttgtccccacaagaaaccattgacaaatagagctagggaggtcgctcagtggatcagagtgccttatagcacaaacataaccagtagtgtctgtgtacccttgtgaccctagtgcttggggcaaaggtcagttggaggcagaagaatggctggggcttgctggcctattagtttagccaaaaaagtagcaagctctaggttcagtgagggaccctgtcttaaggggctaagggagagttgacagagaggatacctaccaggctgccagattttcagatcagagttttctatgtcactagtctgatatatcgtctaaacttgttttattgaaaacaaatttgtcaagtctgttttaggaaagtaaacttgaaaaatggaactatagttgaacgttctattttttatttgttgctatattttcacaaagtgaaacccagcctagatagatggagatccattcttcaggagactcctgtgcctttcactagatagtagctccttcctaagagaggtaatcacctgtgactttatcagagatatactttgcttagcctcatttttttttttttttttaaatagccagatcctaagtgtgtgcttgtttaggttcttatttcatattcttaatttagtttctgttatatgactatactgcagtttatggcccctgctgctgttgatacttggattctgtacagtgtggggttcataaggataattttgctatgacagttcatcttcctatctttttgtatgctctcactcactgttggtagtagatgcctaggagaggtgttatttggtcagtaggagtgtgtatctctgtaccattttatgtttctttttccagaggataaaaattacagttgcagcacatgcttgcttgcaattaatattatgtagttttaaaataaacaaaatattttggtatgcagtaatatctcactgtgataacaatctgtatcttcctgattaggttgaatatgatctttagaatttttattagattcttaggctatcttaaaaattagattgccattttattttaaaaaccattccacttttgattatgtatatacatatatgtgtctgtgcgtgggttgtgcatgtgagtgcaggtgctcatagagtgagtacaggaggatatggaactcatagctagagttataagggggggggcactgagctaccgtgggtgctgggaactgaactgaggtgctctgcaaaagcaggaagtgccctgaccacagagctgtctctctagcatcagttgtcattttctttctttcttttttttttttttttaaagatttatttattactatatgtaaatacactgtagctgtcttcaggcacaccagaagagggagtcagatcttgttacggatggttgtgagccaccatgtggttgctgggacttgaactctggaccttcggaagagccgtcgggtgctcttactcactgagccatctcaccagcccagttgtcattttcttaatgatttgaaattgttttcagcatgttttggatatgaatcatggttgtacatgtgccttgccagagtactgtatccttagcctgcattttcagtttttcataatgaatatggaattataccaatctagtttttttccacgtgtgccagttaggaaatctttgcccaatttgaaaaagttataaccttttctggtcatcttctaaaaactttatggtcctaaattttctgtttaagcttctaatagccccctttctttctatatttcccacggtctggggttagatgctatcaatctaattgctcttgattttagcacaagtagagagaggaaatctattgatatgacttaatatcatttacatttatttaatttggtgtgcatgtatgtatgtgctgggtgcatacatgtggcagtcagaggacaacaggataggttccttcatcctcgtgggctctagagatgaactgaggtcatcagacttggcttctttacctcaggacccatcttgccagctcaagaaacattttgctgccaggcatggtgttgcacgcctttaatcccagcacttggtaggcagaggcaggcggatttctgagttcgaggccagcctggtctacaaagtgagttccaggacagccagggctacagagaaaccctgactcgaaaaaaccaaaaaccaaaacaaaaccaaaacaaaaaaaccaaaacaaacaaaaaaagaaacattttgcttttgtataagttttagatggtagcagttgtgttgttaattaaatctggtgagtctgtaaaaggaattttaaaagtttttccactttgattaagaggcagccagttatactttatattatagtacatccttttgagtctagtaatggtggtgatagaagacgtgtgttttcttttgaggtagtgtctctagcccaggctggccttgagcttcctattgaactcctgattttcctgtgtgtacttctaggaacctgtacccagcttataatggtagtgagaacacgaactgtgcatcttggtgtggctgctgtgtgtaagcaggtgacttagtcgctgttaggcatgaagtacttaacaacaaaaagataactatatgctttgtaaaggcccaagtgaagaacctgacaaatagtgagtttataactcttattcctgaatttttgaacctaattaaagttgaaacctgactgagaagatttgtactctagttgttcttattaagatgacttaaatctaatttttataagtagtaaaatcaacttgccagtttatacatatctttttgctatggataatatattttagtaaagccgggcgtggtggcgcacacctttaatcccagcacttgggaggcagaggcaggcggatttctgagttcgaggccagcctggtctacaaagtgagttccaggacagccagggctacacagagaaaccctgtctcgaaaaaaacaaacaaacaaaaagcccccccccccagggtttaaactactaatatatatatatattatatatatattaaactttgcatgtgtttttgtagtttattatctcttccatgcattatgtatacaagttatttttaagatcataaaaactttaggcagactatgtttaactctgacattgaataattatttttattttctattattcaggaaatctcagctatcaaggagacttttaaaaggctctctaaatatttggtaagtgcttgatttctgtgtacttttgcatgtttcttatatttatgtatatgctacatgaacccctcttgacagcagtaagttaaattacaataagaattatatttgttttcatttttacctaggtacgagaatttcttctctttatatcttttgtgatccctctacacattgtgtctagacaatcaggttagtaagtaggaatagtaatttatgaattaaataagactttttctacctgagattttataataattgtctattagttgccaaaacaaacagatgatgatgatgttgtttactgtttgttgctgactgttccttaatgtattaagtcttgtttattacaatgtatgagcataagcaccgaccggtcctgtgggttggtgtctatgctgtttcccatctgaattaccttaatttctctgttctaatattaaaacgaaaacttctgtttgaagttgcctctaattaagaatttggtaagttaatcatttcactagggtaagctatcccctcctactagccccttgtgtacatagtatgtgtatgcggtgtatgtatgtgtgcacatgtgagtacaggtatgtatgtaggcatgcatgctgaaaccagaggaggatgtcaggtgtcttgctttgtcaccttcttccttcttctcttgagacagagtctcagtgaaactcaggttcaccatcttttggccgagctagctggccatcaagccctaacagctttttgtgtccctcaacccagtggtgacgttccagggccacttggccatacctaccttttatgtgcgtgctgggaatcaaaacccaggttctcatgcttgcagagcaagcaccgttagtcagaagccatttccccagcccatctcttgtccctgcctcttttaggctggttcctgcctgtccatccatgcatccaccatacatgtatgatgcatatcctgcacataggggaaagcaaagcaggtgatagtttgtcttccctgagtttagcataatcattagttaacatgatgatttcatttctatctatcttcttaatgtatttttattcttctttaatagctgaggggaaaagaccccctcatagtttatgtgtgcatgtttctccatccattcattcatctgttgcatggtggctgtgaacagtgctgcagcaagcactcatgtgcacagcactgtgctgtgctggctgcttcttttagctatgtgtctaggagtggaataactggaacacaaggagttctagttgtaggtttttttgaggatcctctgtactgatttccatagtacttataccagcttatatgtatactaacagtgttgaaagcattcctttttactagtgaaaaaaattcaaaaggattaagtgttttataaaatgggataagtgctgagatatccaataatcaagtaccgtggtatagtttatggttgtttttaatataaccacacaaaaatgttttgttttgtttttcattcgctaaaggagtgtctctagatatatgaagtgggaaaaacatctgtatttgaaacagtcagttttcaaccaaaagatatttctatagcaattatagcacatgcttacatgagcagcagttacagtattctgtttagcatacacctgcaatttgtagcatattcaggaacaactaatagagcaaagatgcctgtgaagatttccactctgtgtgccttgtggtggctcatctctggagtatatcacacagaaggggctagtttggccctctcctaaacatgaaccagctgtcatcattgtaggcttttggagcatatctcatcctaaatgtatttgtgacagttttattgtcttacagtttacacaccagaaaacgtcacacttgtgtactttccaggattctgagtctgtcagctatgcactcctcacagccactcaggcgtagggggctttccttactgtctaacctcatctccctctcttggcaacaagtaatgggctttctgctcgagagtttgcctgttttggttatttataaaaatgcaccacttagtatttgttcttttgtgcttcattcacttagcacagtatttttagcatcatatgatatgtgtcatatcagtacttaattcatttttatgacttggtattcctttgtatcccacatgttacttatcctttcatcagttgatagagctgcttttttggttgttagtaatgtgtattgtgaactagtgtggatatatattttcacttcttttgagcatgtgtgaatggggaggggttgagaattaatcctttgtgtttcttttcaagtagtggtcaaaatagcaatacactggggtcctagtctccacatccgcatgggcagccatgtgtgtattttagtcttcctggttggtgtgaaaggacacttcattgtgggtgcttactggccgtttgttgtctctgaagaaagagccattcaagtattttgctcattttccagagcaaatacttttaattttgaattgtgatcattttttttacgtgttatgagtagtagatgtttattcagtatgagagttccaagtattttcttcttttctatggattatccttttagtttgaaaaaaaaagtcattggaagaaatgacccattttatcagatgggtttctctgtagccctggctgtcctggaactcactctgtagaccaggctggcctcgacctcagagatctaactgctctgcctcctgaatgctgggtttaaaggtcgtgacaccattgcctggcagaatgaagtgaaaggttttaatcttgatagagctcagctttcattttgctttgcgtgtggcacaaagttacacgttcattttaaaataagtcattattgatagtttaggtctgtaataggcttcaccctaagtccataaagaacactggcatttcttgtatctgacggccagagcacaaagggaaggatttctactgtatttgtctttcagttccccatgcatttgtttcttatgtttgactctgtgattactcaatacccgaccctacctgcccttgggggttggaggttaggtagtaaagagtcaacaacacaggctccaggagcaggtgagaaacaccgcagcaagctcttctgaaaactgctgcaagctcctttaataccctctacacacacatgcccattggtcccaagaacagcagttcctgactggctggcactgtctgcatcaatccttgctctctcaggcagtccgcacttaggtcctcctgcaggatatgcttttgcggctccttgccccctcccggcatttacataaaggtgaccccgctggtcctgctacagttcctatatgagattggtttatttatattagtctgtgttgtatatgatagctccccctccacctgagatgagacagggtcttgtgcatctctggcagatcctggagatctctgcttcctaagtactggggtttttgaagttttctttgcatgcctttggtttctgttctggaaagaaaatagattcataggagacttatctttgttactttttggctgagaatgttcttctgatttggcatgtggccagaagctttctgagagcctcacagaacacaaagctgatagaagaagtgctgggaagaagctctgaggcaagatggcatcagaggatgtgagccatgtatctgagtacagtctctgcccagttctttcactgactaccataaactactcaggtgccagggtatcaggctgataaataggaaggcgtggctcaaaggtgaaggagctgagcacaggcagtagctacatgctgcaaaggacagtttggtgttcccattcacaacactaaaaccacaagttcctctgaaagattatagcaaagtccagatagactcttacattgtttacataaggaattctgttttcaaagggctcaagaagaatacttaggagtgaatagataaagcatagaggcactttaaagatgcaggagagagttttagggctgtctggaaggagagttaattaagtaacaaaacaagtgtttagcagtgaggagagtcaattagcttagagtaataaaagccatcaactggaaggatgggaggatgaagagtccaagtcagaggcctgcgagaggattgctagcctgtgatggcacaggaccataaataatggctgcagactcaattcagggggaagtgtcagcctacaaaagcaggaagttcaggcagaatcaacataaaagagtccacatttagctaagtaagtggtctagctgcttagttgcagagaaaatcttgtaagcactaatagagaaagaagttatcatgttggctagttgaggaactagacattcagtgagctgaaagaaaacctaaaagatgcgaatttgttaccgtaatttgaaagtaaaataaagacatgttcagataaacactggggttttctatcatcccaccacacttatgaacagttctataggaaattgattggtttgaagggaaatagccccagaagtcatatcgaagctagtaaatacataatgtactatatttttctaattataaaaagctatatgatttccttactgtaaaaatgactaactataatactatattgttgactaatgaaaacatttatcaaattatactgtttctttttgactaaatttggatttttttgttgtcgttgaggatttttattgtgcttttgcttatttgaatgtatctcctgtgaaatgtctgagaaatgacctattagactctttaataagatataccatgacaagtggatgttaaatgatatttagtctttcaacatctcaaatctgctaggtctttaatttaatttcaggttgtttaatagcatattgtcactttgaaatgtgagaattttctaacaaatctgacattgtaattatttcaggtaggccattcagttgattgcatttacactgcagtccatatgcctttcactgcctgtatgcagaactttctccttactcccgccagaaactctgttcccagtaagcaacaatcccgtagttcctcttctttaagtgttattagaccgtctgtctgagccatgagagtgctgtaaattctcaacacgagctgctcagacagctgtttgcagactcttctcagtttgtgactgattcttcagaaatataaaagctttgctgctgaggcctcagaaggaggtaagggaacctgggagagaaagcgggaattggggctagagagatgatagaagaggtgaggactcttgctactcttgtagagactggggtttatctccctgcaccaacattaggcagctcgaaacttcctctaccctcctctcagtgccccttaagacatacatgcatctgcccatacaaacacacatgcacagcactagcatgctcacacacaaataagtaaataagaatacttaaagagaagaaagctttttttttttttttttttaaaggaatcaatgaaatgctcatgaatagactgctggtggagatgtaaatgttaaaggttggtgagggtcacacgtgtggaggacacagggcctcactgaatgtagactttcccagtgggttagactggctggctagtgagcccctcatgcttctgcttctacctcctagtctgggatcgccccaggattgccattacatgctagcctttttacatggggagctgaactcaggtctccatgcttggacagcaagcattttgcctacctagccatccccttagcccctaaatttatagttttcataaaatgtcagtatcacttgaaacaaacttcttttagagctggggaccaaacctgaggatttgccagctctaccactgaggtgtttccctaaccctgtgtgcttaggagttaccataaagactttgtcacttctttttgaacacatgaacattgtgcagtttaatgcaaatttcagaacttccttgttaaaaattgaatttatgatagaaatttatttagcttaatatattagtctacttagattagcttatttattgaggaaaattatttggaaactggaagtacattgttttttggatcattttaattggtaacttctgataatggcagtgagtacatttgaattataaaatctatacagtcagcccttatctacctttaaatcctttacagtgtggtcttgtttgcttgttttttgatggaatcatatgcaaggatataattgttgtgtttttagccattactacattgaaattcagtgtctaggtttaaagtaaaatgtaatgttacttagtatgaaattactaaacattacagttccctcttctgtcacttcagagttactgcaccatgcttagctttaaatagctgaccagaaaactgtgtaccaattgtattgttagtatttctttaatagttgaatttagaatattcactgccaaaagttatttgtaggtaatatgggagattgggtaacaaggcaagtaacacagtgaaaaacacaggcattgtacaattgatggcatcagcatgtttctgctggactgggcagggcatctaggcacccagcggggtggattaaggcgaaggaagaagccggtggaacggagtgctactccattcatctgcgcggacttctttactttagatgtttttgagacaatgtggaaaatttgactattgactggtgttaatgaaatgtggttaatgtgatctaattgtgtggtgattctgtgaacatgagaggcacttgtgaagtgtgtgtgtgtgtgtgtgtgtgtgtgtgtgtgtgtgtagtaagtaggcatggtatataggacttaatttaaaatatctgcaaggaagaagaaaagtcaaagcaatattggtgatattttgaattgaatataggtgtaatagatataaaatataaataatatactgtttttttaatgtatattggaaacttttcatgtaaaaattccagatttttacctactgactgttttcctggtaccacaaatcctctcctagcatttacaagtcttctgagatattggagagctttacttaaatttttgttgcagaaatgaggctcttgtaaaccatttaacctttccctttgtattttcttcttgttatctgtcaccattgatcatagtaattattctggttgttacgtcttttttttttttaaactttattttgtctttgttttgggaatataaatttaattctggtggaagagatttgtttctgttgcagtaccttcattacaattgttttgttttgtttttcgagacagggtttctctgtatagccctggctgtcctggaactcactttgtagaccaggctggcctcgaactcagaaatccacctgcctctgcctcccaagtgctgggattaaaggtgtgtgccaccacgcccggctccttcattactcttagttgcctagcatattagatgcttaataaatatttcaatgattgagagaatgggaattagttctagaaatccaaggacatggagtttggtcaaggacagagcaaaagagaatgtttttgaatcttatgaggctatggaaaccttaacctttggagaaaaatgtgggctaatatgtacagatttggaagagtaagtcaccacctcttgttttttcactctaaaaccttgatatttcatacattcagcatagtcccaacaacaggaattcctctaagttattgtgctgtgcttttccaattgtcttcaaaatgttctctgactttttgaggcaaatcttactctgatttccaacttaacatggaagcggtcctcctgctcagcctctcagctctccaggacttcctgcctgtgttgccatgactgtacttatttatctgtatgatgtaccacttgagtctggtgtctatgcaggccagaagaagggattagtacagatggttgtgagctgccatgtgggtgctggaaattgtacctgggtcctctggacgaacagaacagccagtgcatctctctagccttgaataactaactaactaactaactaactaactaactaactaacacacacacacacacacacacacaaaataactatattctccttaaacaaaataagaaggatggcattgtgtttcatttttgtaaatgtatttaaggtctggcttgaaatatgttttagcttttgtgtttagccaagtgagagacaggctgtaaagtgtaaggaaacttctcaggtaaataactacaggtgtcttcttataacatagattgagaatgacagactaagcaacatcctaggagtactgaaaacagttgtggtctcatagattccatgcaaggatcctgggaaccctccatgttgctggagtgaataatttctcactgggatactgtttatgaacaacatgctatgtttgtaaatggaagaatcattgtttaaagatatactagaaccggtttggtgaatttatatttgagacatttttcttaaagactgttgacagtaccttatgtttacttacacagataaaaatgggccaaagaatgtcctgccacaaagttgtgtttgtagttttctgaggatggatgaatggactgatggacagatgggtggatggagggagcaggggagggagctggagatctaaccaaggcccttgttcatgctaggcaaacactcaagacctctactacatacagtatttaacttctcaaaggaagaatttgacattgagttacttatagttaagaaggaaactagttactttattgaagtaatcatgagggctggggatgtggttatgatggcttgaatgagaatggcccccaatgattcatatatttgaaagtttggtccttagtaggtgaaactgcttaggaagggttaggtggtgtgggttttgttgaaggagctgtcactaggggttgggctctgaggtttcaaaagccaagactaggaccagtgtctctctctgcctcaaacctgttgacaagatataatctgtcagctccttttccagggccatgcctgcctgctgccatgcttcatgttgtagtagttatgagctcaccacctgaaactaagcaagcctccagttaagttcttctaaaaagttgttttggtccttatgtcctcagtgcagtagaacagtaactaagacaacggtatattccttgcttaccatgcttaaagccctgggttccatccttagcatcacacaggaaagaaaaatcatacttttaacttattattgataaattattaataaataatgttttctctactttttaaaagggcctgtaggtagacttgattaaatatgcactgtctttttccctccagaacagtattatgctcatttttctgatagttaataaatgtttacttctgttggttagtcttcaagttctgtctgtatgcaacatagtgatcagtattacattgaattagttaacagggaagaaaaatgataaataatgaaataaccattattgcttacattctatttttgttagaactaatttaagagccaagggatattggggatggtatagggaatgaggctgatgaagcatttcatgcttttgggtatttcattttattttattttgtttttcaggacagggtttctctgtgtagccctggctgtcctggaattcactttgtagaccagggtggcctcgaactcagaaatccgcctgcctctgcctcccgagtgcagggattaaaggcgtgcgccactacgcccggtgtggatcttttattttaggcctttgtagaagcagtagtgatgctgaggctggtcagatacctggctttcattgagagcagcatatatttgctggggactttaaattgattcttgctttattttgtacacagtgatgtatgttctgagcatattctgtacttattggcggagtaaattatgttagatgttaatgcttggtacctgttcttacactagcacaatactttatttatttatttatttatttatttatttattttaccttctttagctcccctacttcccatatttgctagagaggtttattagcagcagttaagactgtaccctaatgtctctgatgagcatctcaaaaggtagcaacatggctaaagcttgcttttacaagttctgttgataaatcattttagacacttagtctgatagtcccttgcattttccatgtactgtgttctttggcttgtctggcagcagcacaggaaggtagacttaacggcagtccagctcaaatgcatctacagatgagaatttatcttctgtttagcatacagtgttttcagcaggcgagtacgatgttactgaattattttgcttgtttctttctacagtcgttatttatagaacttttttccccccttgtgaagtacatgcctgtttctaggtattctgaaaaaactgatttaagtataacatgagttaagtctagaaggacagtgcaaggtgaaggcagaaagagtactgactctttttagctctaaagcagggaagggactgagcgtgggaagggactgagcgtgacacacacacacacacacacacacacacacacacacacacacacgcagagcagggaagggactgaatgtgagacacacacatacacagagcctttcaatgcctcaaacataggctcttctatagaaggtattttgttatttttagtttcctttttctttttcttaaaggtgtattaaaaagagttgacatcattggtatctgaatgttcattaaattattttgaatatgattatttaagaagactaacatgtttgtattaatgtgactctaattctgggaattcagaaagaattataattactggatacaagtaactgttatcaggaaaggtagaatcttttaagtataaaattgttagggaatgactaataccttcagtggtgtttgcttatgttttcagtgtagttttcgggtctgtctactgaaatgcaaatgaccaagggaaagatgcctcgtcagcttaggttccgatgacttttagttattgcaaatagagcacatagccacatccttttcttctccgtaaacctcagaacacttatgacagcaggcattccacagctcaccttgctgcttgtctcattcttacctgtagtgaaactctgcagtagattttcttgttatccctcaggtccaagttcagcatgcagagggaatttcttttaacattgtattcagtcatcctcctgcctctcccaccactattcaaagatttctgctagctactctccacattttattatttttaaaaaagctgtaagcccacgttgtctgtttctcgttgggttttagggtgactgactgagaacagttctgcctgtaactgcaatagagaaataaagcgagtgggtattcttactgtgcgtgcttggcaggcaggcgtcttaagtgtagagtcagaggtcctcagttctgagtggtgagctagaggctgtccagtgatttgctcccgctgtttctcagagcagtgctgcagcggttctctccagatcctgaggctttcttcaacagtgtagaacagtgagggtggggctctttccggttggctgttgaaaaagcgtccatttacagagagaacaggttggataggctaagctgaaggcgtaggcaaacagatccaaaactgatggaagacagaaagagtggttagtatgttttggggtgtcttgagcttgcttctttgctaagttcttgagctttaatataaacacattgaaatattaaagcactttacattctttgtgtagtagcctggagacttatgcttttcacagtttcttggtgatgagtttgtggctgctatcttctatagtaaagttggtttttcatttgtttttaatttgcttttagtggtgtatctttcagccagagagctgcagaatgagtcagttagccagttttagtatatatgggacattactagttgtgattttttaaagccagattctcttttaatgatcaagtcaatgtgtggctgtggctgcagcaacattatttcttttttaatgaagaggttgaaccgttcgacttgctgctccgatcttttcccttgtagctatacatcagaacagttgcagagattttccatcgaatgtcccaggcaactaaaggtaactacatagatttatttcaaataagaatacacagttaaaatggctgcattataagttgggccaaatgattaaacataaaagaccaattaagatgttcttagcagttttcttgaccttgcagtagctatccaatatcattttgtcatcatcagattgtggcagtggaaatgtactgcagtaattgattttgtaataggatcaggtgatttaccagcggcactgagaaccacttgcttgctctgagtgtgcagcactctaatgactgtgtgattgcagcctagagctgaagtgaggtggctggccacccacagtagcagcgtttactttagaaatcacaagagtactaggagtttcttgtttgttttctgtgctatcagttttacttcatgtgttcatgtgaagtcttagaaattacgattttctttttcactcaccagttcataaaaagtttatcggtttgactaacattaagtataacaaaacacattcttctctctctacttctgagctcagcatcaacatactgtgctgctgaccatgaggacacatgtctaaaacataagcttttgttaactattgtacattatctgcattaaggaaaatattcttaacacctgcaaatctcatcatgtttcaactcttatctgtatggtaatactgggactagaggaaactattggtccagagagttactagaaaattgttttgtgttttattttgtgtttggatttctcagtagtttatttagcaaatctggactgtataatttcttggaaatatccagaagaacattaagggcaagtgtaatgtttaatgatactacaaattaataacttggtatccaattttatttaatatccaaaatttataaaatggctatgttttaataatgtttgtgcctttaaagcaaataattggttacaaatatgtgtgtacagagctcattcttgatgcggaatgcacctgctgttgtaggaatgtgagaccctgtgttgagatcctggctgctgagattttgctacaatgatcttttgcttagctttgttgtattagtgaaaaaaaaaattacacgctttccctccctcactcctttccttgccagaaccaccaactcttgtgcttagtatgtaatagtgttttgtacagaaccacacacacagggaaaatgacttttctgtcagtccttagatatatttttcctgttagccttactatatttcataacagttaaaattagatcttgcagaatcaagaagagttctgccattgcatatgctatgtgaggcgcttccctttctgataagcccagaggctagttcttaaactgtttgaaggcttggtgtttcttgaaaggtgaaaatatgaacagtcatactttggccaaatccagatacttaaaaaggtaattatagttatgtgttctgttgctaataatgatgcaatatcaacatggagtttgtagtctttattcttctttgccaacaacagaaatccctctgtatacattggtaatgtgtacataaggatgttattactatttgagatttgttcaaggacatgtctaagttcaatattgaaaatgttaaatgatgtaccatcaccaaatcaattactgataaatatttcactatggtagtgttcattaaagcccctcccccagcattgtacacttgattgagcaaattggaacagaaatatgtcccagtgtgtgattgcttcttaaaataatgattgtgaaagacacagggcactgttacatcttcaatatgatagggcttcctttagggttaagtgggagatgtatcctgggaagtccttgccccagctgggcacactcttccaaagtgcccgagatcaaacacctggattttgtttgactttgttacaaaggaatttgatgatcttgacacagttacatatgtatgaacactataagctcccttaaaatcctcattagaaagaaatgggcgtgtcctgtccctttagcactggggttggaagctgtagaagttggactttaatggaaagaatcagtgtgcatgctgtagggacaactcctttccatattcatgcctttctttttgctgagactactgttttatctttttgatatctaatattctctaaatagcatatttgtagtagcgtttaaaagaccagacctagggctaaattctgacacgtgatttttttttccctaggcatttgatgataataatgttgttcggtcatttgttcagtatttcacatggtagttttcagtggtcagagtttgggaaggcactgttgtttccagtgctccagcaagtgcacctggcaggtaagcttgtggaggcgcctgcctttgcggcttaggatggactgttgccttcccagagatggggatgtggtgccgactcactcatccttcaggaacctgtgttttggatcatgctgctctctagtgaggactcctttgtcagtcatttcacttgtgagattggaactctgagtatttttaagacaaagagactgaggcttacaaaggtctcattcctaaaatcagataactaggaaatagaataactaaggttgaagcactactttaaaatactttctttgacaacaatgtgataaaacactatattttcttacactttaaaagaattttctttcagttaagcgtaactgcagagatggcaacaaatgtctgaagatactcatgtatccataaactacttcctgtactatgagtgaattttcaggtgtgggttagattttatcaactgcttctctctcgaatgacagaaaagcttggcatttactaattgtacagaagaaaaatataagttcatgttaatatgtgaagaatcaattaacaaacaacgggactagaaagtacagtttgtccttctataaaactattatgtagtgtgagttggtttgtgcattcctcactgagggatttagctggtatagtggtaaagtcacttcagtcccccatgttcttaagtaatgtaaatacatctatttcagggtttctactgctgtgataaaacaccatgttcaaaagcaacttggggaggaaaggggttatttcagcttacagttttacatcacagtccatcactgagggaagtcagaacaggaactcaaagtagaaacatagaggcaggagttgatgtagaggccatggaaaagtgccactcactatcttgctttccatggctttcttataccatctaggcccaccttggggtggtatcaccagcaataagccaggcactgagatatcagtcaagaaaatgccctgcagacttatctacaaataaatcctgtggaaacaaagactgtagcttatgtcaagctgacatgaaaattagccaacacaccatcagaagtaagtttttttcacaaagagggtattatcaatatataatggcattaagaataaaactgaaggtccagagagacacttgctatcatgcccaattacctgagttcattttctacagcccacatgataagaaagaaattcagagagactcctgaaattgtcttcagaaccttctgaatgccacatacctattttcagaacctgatcttgcagaatttccttctttttcagagaaagacagattgtttattatttgtgtgaatgtggaacatgtgttatctaagtatggatatgtatagaatgtgtatacacaggtgtatgcatggagggtattaagtatctttctcagtctatcttacctctatattttgagataaagtctctctattagtagctttccatgtcctgaaactcaatatgtagacaaggatggcctaagactcagaaatctgctcacctctgcctcccaagtgctgggataaaaggcatgcaaccaccaccatgcctggctcaagccagtgtttcttactgatcataccaatttagctacacattttggccagcaagccgcaggaatcctcctatcgccatctctctagtcctgggattacagatacataccagggactcaaaacccaggtcctcacactcatgcggacacactttacccaccaagccattcaatttgtaccatatgtaacagaactgcctgtgttacagctttcaaggatctcagggatagccagtgcttcatttagacctggctttactgtggaatgaataccatcttccttgagatgaagttttattcctgatgttcatcatgttcagtcaacctcaaatagtgtccatttcagctgtagtctaagaaacactaatagagatagaatgaaatattaaaagaaaaaacactagaaatacagatttaattatagagctaggatctgtggtccttttaaaacaaattaaatttaaattcttgtatacgctgtctacttttcaaaacaaagtctggggctggagagatggcttagtggttaagagcactgactgctctttcagaggtcttgttcaatttccagcaaccacatggtggttcacaaccatcggtaatggaatccaatgcccttttctggtgtgtctgaagagagtgacagtatactttgtgtgtgtgtgtgtgtgtgtgtgtagtgtacacacacacatataaataaataaattaaaacaaaacaaaatctgagtcagtttcatcgactttatgaacatcccatatgagaaaaaaaggctatccattaaaatgatatattttcttgggagccatctgcctttagcagaacaaaattgaaattgaaattattttcttcatgaaaattacaaattaccaatgagaagttatgggtaccattcctggttataggcaattctttctttaactaagctgaaaagtaataaagttgacttcaatatttaaatctattgattacatatacttccctcatccaatctaaatcataactttgatgatataaatccttaaaaagtaaatcattaagatatatacttcaaaagtaccatttgtgttgctataaactggaatcacttaatcaagaaataagagagaaagaagcgtgtagcacttcagaacacacgaggggcagcagcctgaagagaagacgtacccacgcaccaaacattcccagctcacctgcctgcccagattgcatcctgtccactgcacaggatttcctttgccttggaactgtcattcataccattttctgtcaagttaagccctcttctctaacattttgaatcagcttctgggttagggattaaggttagtaactattcccaaactccatctttctttttcattgcttacatttacatgatttaacttctcaaagcctcactataaggcatacgtcttttctgttaaaaattttatatgtatacatatataaaatataataaatatatagtataatatatatgtttgtatgtacatatatatgtatgtatatttcttttggggggcatggtttctttgttagtggtgtggtcttcttttttgtttcttgagatctcacagtgtgaccctgtataatcctgcaactgctatatagaccagctagccttgaactcagagatccacctgtatctgtctcccagtgctggcatgaatgacatgaaacacaccacatctggtctctgcaaaaaaattaatgtggaataagcaaacatatatctttcttacagaaaaataaatatgtatcagtaattaaaagcaatttccactctggataaaaacactaataaaaatcaaaactacaatctgcacagaataaaatgtttataaacaactagaactcatcctactaaactaactcttaactaagatgactggagttttcagaactgggtacttgtcaacatgggtgatgtgccagtatcaaaactcaggttttttgtaactcctttcttttcccatatcatttacaaactaatgacatcagttcctttgaatatcttccccacaccagaaactagacccaccaaccgttcattccccttctgctcaaaagctctacctccttaacctcagtcactctgctatgcacaagtctatgacttaaatgccaagacagaccagggtgaatggtgaagtctcacaaagaggagagaatttgtaccaatttgactattcagatttaatttttttaatttgattttcttttcatttctttgatttttgtttttaatgaaaccctaataatagaatgaaatgttagaaaagcaaaaatcagtattaaagtgtacattgagaaaaagctgtagtgtcctgactcccaacctctgagtaactaaggactttcaagttgctcctaaagttaacctggatacaaggcacatgttaaagggtgcctctttgggtggcttagcttacacagaagcaacaaatgcagaccttagattctagaggatgccacttactgctcttaacatgtgcccatggaagccacttcccaattcttcttgccaactcagtattatatatgtgtagaggaaatttatcataacatccagagtaactgtgtgaattgaacacaagacaacacacacacaaaacaaacaaacaagaactagtcatgtctgaagctggaagtaaagtgtcacggctctgactcggctcctttcccttaaacactgtttttgcacccactgccaattcgtgaattcagctccgcaacacttagctccttgtctccagtactcgtccctgcaaagctgctgcttttggcttccctgccctgcatgccttccccaggctctccactctgctctcaccgtatccgctgccaccaacacggctgattcttaaaactcaacgagatgagccatcccatgggagcaagggcactgcttccagggcagattcctagaacttgtggcaaaaacaaaaccactggctccttctccactgctgtcttccgagatggctcgcacactccccactccaaagccattaaagggcgcttaagaacctcaagcagcatcagccacacctgtgctcttcagtgttggctttcctaaagggcccagcagctgctccagggcagtttttgccttttctgtttcagtaattccagttgcaccggaatttaactgtaacctaacaaagtaagtgacaagaatggcatatgaatcgccctcgtagcagacatctacagtggttggataaaatgaaaaccatttgcttattctatctcaacaccatcaacagtctgaagcacgattttacattttacaaaacggattctttacaaaaacaatccttaagaaaaggaaaacttgcatgcaacatgcagaacaagttaagaatgacagtgtgttctcagaaccaatggctctcaaaagaacccagggcttgagagatggctcagcagttaagagcatatactactctagcaaaggacctgagttcaacatctggcatccatgcgaggctgcttagtgggatccaacattctcttttggccgctgtagacactgcacgtatgcagcgtagactcaactcatacacataagtttttttttttttttttaaacaaaataaagatgcctgtatcccattacacagcttttgccagtggttactaatatagcgtgaatgtgtgtacgcagatgtgcatgtgtgtgtaggcacacctaactctgcacgtgcatctggaggccagagaacaacctgagctgttgtatctcctcagacacagcaacagcctaccttgtctttaacacggggtctctggctggacctgactagatggccggtaaacaccaggaatcttcttgtcttcttcccagtgctgggagctgcccctggcctgcccgtgggtaccgggcatcggcgtcaggaacacatgcttgaacaccaagcacgtgacaggctgagctatctctgcagctctagtttttacttccccctcctttctgagtgaggcacgtgatccctgaagcactactccaaaggaggacatgcggtgagatatgtttacaagacaaattaccatgtgactgctgaataagagggtcagcatgtcacctcattccagtcgctgtgataaaacgcccttaacaaaagcaactttttgttaagagaaagggctgattttggctcacagttgatgatgggctgtcatgaggggaccctccagtgacattcaggttacattcttgctgcccagcaatgcttcccctttctgttcagttcaggacccagcctgtaaaggaatgctacttcctaattttagggtgggtctttagagcctaagttatcagttaacctaattgaggagacaggtacaggcaagggcaaaatctgctttcctgggtggttgtaggtcctgttaatttgacagtattaacactttccaaatgctagtggaatcagattttccagacatggtattatgtacacatactggtgtgtccctgcatctttgtgtgcacatacacgtagaggtcagaggtcggcctctgaagttattcctcacacactgtccaccttgttttgtttttgtgacaagtgagctccagggaccttccttctgtctcctgctgggagacttaaatttccaaataggttttaagatcagaagcactagtcgggtcttggggtctgggcattgagcacaggtcctcctgcgtggcaagcactttactggttgctgtctccagccctacttcttagtaagttagaaaagtcttctgtttatttacaagcacaagtagaaatgattttctgacaccttacagtttgaatgcctataggctctcataatagcccccctccctcactggttgagtagtgaataaggaggtgtgttaaagtaaattaactgacttgaagatgaatgggaatattgtgccaggtagttttcagtagacacattaggacagacatcacccattttacaggcgatatgctctatcagtttctgtatctcattcttacccttgttatccggctcccttagctcttgcctgccatctgagggaatttcaaatgggtttacagaagagctgagcaagcagtttattacagtaaaagttcctccagacagctaggagagggctgacaagagggatggcccacagctctcaggctatgaggtttgcctctccccccacccccaccccccatgtcctctacttatgtccccttcctcccaccccttttcctctcccctccttgagccagggttctataaaacaggttgtttttatcttctcccagtgtgtgcactcattgctaagttgcttgtgatctggtgtcctgatgataggatcacatgactgtttgtcagctctgatctcctgatgccacagagctgagattaaacaatgcagctttaccccctgaccccttccaccactgttgactgtgacttctacccatgcagtaacaacttggaataaaagtaactcagataacattgacctgatgaagctgtgggggagtccttgtcagaatgacagatctcatcatcgtttgcattttgctgtcatcttttcttgagcagtggctttaaagtggtgatcaggtgattctattcctaggcagcttagctcaagcaaatttaactttctctgaagcagtataaattccaccaaagacacaaagaattcttataaagctccaaggtaaaattccaggggacagtgaagcttcaacaacaagtgaattgaagaaaaatccttgttctagcatgcctctcatttgccttctaaaaagaaacagaagaatggggaagagtctactaaaaactgaagaagtatacatatgtaaagctcagagaatgtcagtcaggataaatacaagtaaactcactctcagactcagcacacagtaactgcagatcagtcagcctaaagaaactatttaaactacaactggagagcagaagtaatttacctaaaagaactaggtatttcactgcattgtgagtagaaatagtggaagcctgatgatggtaagttcagcttgctgagaagggtggccaaatgaagacctgctgtggaggcagggaggctatcttagttagggtttctattcctgcacaaacatcatgaccaagaagcaagttggggaggaaagggcttattcggcttacacttccatactcctgttcattaccaaggaggtcaggactcgaactcaagcaggtcagaaagcaggagctgatgcagaggccatggagggatgttctttactggcttgattcccctggcttgctcagcctgctctcttaaagaacaaagactaccagcccagagatggtctcacccacaaggggcctttcccccttgatcactaattgagaaaaagccttacagctggatttcatggaggcatttccccaactgaagctcctttctctgtgataactccagctgtgtcaggttgacacaaaactagccagtacagaggctgatgtgtagattccgtctgtattggcttttacccctcatgtgactgctgcttactataccagaggtgtcagaggaggagtcttcatgctgggttacctgagccaaaggatggcagctcctgcacagactacagtgtctctggaaagcactgctctttacagtctgcctgccagtctagttttgcagcatttggtgtttggttaccacgggtttgtgtaggatttgtgtcttagcagaaaaaaagcaaagggaagctgtgggagtcaggcacagcaacccccagattacacggttccttacagtgttcttcccttatacctccattgtaagagggctgtctgtaaagtgggagttgctgcttctactgatagaccactagtgtctggagccaagtaaatgcaagtctaatgtgtgctgagaccacactagaaggctccctgattttcaactctatattttcataggagaaatctcttagaactttttaatcaaaggcaaacaggatgatggagtttcccatagccgttattgattatcttaatttacaaaactgaaatctgaaatgccccagaatccaaaattgttgactgtcatgttggcatacagaaagtttctgatttgggaacagttggacttcttgagattttgaaataaggaatatgcagcatacagcatgttaaatgctgtgtgccaaaatatcggtcagcactgtatatgtacactcttatttaatctttaatacttctataagacaaagtacaatcatttgaaatcctctctgcagattagacagttgaggttttaagacactggataacttgtcccgttttgtaggtagatgctggcacaacttgtgtttgtagctcctcactaccaatccgggcctcttatccaccaagattatagcttgggtgttatgtagctgtgtatttccccatccaatgctttaataacatccagcaaggtttatatttttcatgtcctttgttcctatttatttttactttcaacataaaacagctctgctggaattggaatggcctggctgtggccacagtattgtataacttccacagccttgggagtgaaattcaggcaaatatgttgcagcaggtggtcagtgcttaaaattatttcacttgtgatacagcgtgtgtgtcagaggtttactctaacagtaacattacgctttaactgtacagcacctggcataatgtaaggttcagttggagataattgttattttttttagttgtgtggggaattagaaggcagcaaaataagaaagttggatgtgtttgtgttagaattcaaaattttattttcattattttgccttaaaagtaattttagatgtcaaagtacttataacttatgtaatacagtgctttttaaaatttatctgcagctttaaaaactctttgtaactggtaaggaagcatgcattagttactgttctgctgctatgataaagcaccacgatcaaggcaactgatggaaggaagggttcatttagacttatgttctacaaggtaaggatatagcagcagcagcagcagcagcagcggagagttcacatctgaaccagaagtgggaagcagaaagagggaatgcctgagtttttgaactctgcaagttcagttgcagtgactttctccagcaaggctactgctccccaagcctcctaaaacagggccaccggctggaagccaagtattcacatgccatagactagaaggggacatctgactctcaccactaccaagtgcagtaagaatgaatgcatgagtttggggtgtgcacaattacaaattaccaaaactcattagtcctgatacaagttctaaggaagagagttgtgtcttagttagggttttactgctgcgaacagacaccatgaccaaggcaagacttataaagaacagtatttaattgggctggcttacaggtccagaggtttagtccatcatcatcaaggtgggaacatgacaggcatggtgcaggcagagctaagagttctacatctttatctgaaggctgctagtgcaagactgactttgaaagatctgcatgcctttcttctaaacactgggattaaaggtgtggtccaccaagcctggatttaagtttttctttacctagaacttgctctgttctaggctggccttcaactcatatctatttgtctctgcctcctgggattaaaggcttgtactaccacacctggacctaaatttagatgggtggaatcttgccccaaggtcacttctcccttaattcaaattaatctccttgagcacaggattcagctccatttcacttcctggtgtccctttagtactcaaactttatattttgtatttttcctttctcagcttgctacatttgttcaaatgttcttcatcagacttaaccagagaacaaagtctgttgggctttgttgggacttcctttgtcaatgcaattattataaatctctttagtcttagtcttaggcagtcttttcagacaatggccacgttcaccaaaacaacacaaaacagtctctcaccacatattaaagttcttctccactgaaacctcttgggctagcatgcctaaacagttcaaatgaccctcagcaccagtcttccatattttactaggttggctcattaagccccacttaaagcattccttggcttcccaaacccaaagtctcaaatccacattcttccaaacaaaagcatcgtcaagcctatcacagcagtgccccaatccctggtaccaacttctgtcttagggttttactgctgtaaacagacaccatggccaaggcacctctcagaaggacaacatttaactggggctcacttacaggttcagagtttcagtccattatcaaggcaggaacatggcagcatccaggctggtgtggtgcaggagaagctgagagttctacatattcatctgaaggctgctagcagaatactgacttccaggcagctaggagtagggacttaaagcccacacccacagtgacacacctactgcaaccaggtcacacctattccaacaaggctacacctccaaacagtgccactccctgggccaagcatatataaaccatcacaagttgtcactgtcttaatcttaggagggagcttttccacaaaaggagtagtcacagatggtactatttgctgttgtaagttatgtcttgtttgtggtggaagacagatttatacgagtgacttagaaataacctgcaccagctaagcctctggaccagcagccctaagaacttgtttcctgttccactgcctgggaatacttatctcagagtctagattaggtgaatgtgccccctagtgttagagtgggcactctgactgcttgctggttaaatggaagcatctttacccatttaccaactggaacataaaactggctttgtacctgctaatattttctgtattcttagtatcttggttggtagattaaggaaatggatctatagtattttctgttccataactccttaattgctagtgtaggtaatggatagtattttctgtcttttctttttcttagtttgaattagtgcttggatttttgcctagacaaaaggagtaacttggaatgaagaaaatggactcaaacttaaagagctgcacactaatcaatccttcgaacgcacatggaaaccagagaccacccctactgtcctcttagttacacaagtggctgaggttagtgataaaatcctctgcagcctccactgaattcaagctgtgaccaacacagcattgtctgtatggaaactggtgcctccaagttggagagtgcttacaagtgctgccttgttaggttggtgcagaagtattgttctgtaacctgaacactctgaaaatttcttgtgtgaaaaatacagattattacaaacaggatctattttaatgattcattaagacactgttattttatctctacacaaatggaactctttgtaagagaatcacagtatttcttaccatttcttacctcataaaaccagtcagtacaactgaccctgtgtcaacttgacacatacatccatcactagtaagctataatctttcctttcttaatcatctccagatctataactttaaactccctcattttttacaaattcaaacacattaaaattcccgtccctttaaaatagccagtttcttaaaatccaaagtcttttaagacaaagtctgaattgtggattcaaagtcttaactgtagactccagtaaaatacttttttacttcaagaggaaaaaaaaaatcagggcacagtcacagtcaaatcaaaatcaaactccaactgtccagtgtctaggatctactcataatcttctggacaccttcatagggcttgggtcacttcttcagttttgccctttgcagtacacacagcttgtcttctaggctctggttggctccattccactgctgctgttcttggtagtcatcccatggtactggcatctccaaaactcaagcctcaacttctgtgaaggccccttcagttctgggccttcaactgccactgaggcggcaccctcaccaatggccttttctggccgctcccagtgccaaacctcagctgctctccatgacccctttatatctttaaaacttgtatcgtctgggtgactcatatattcctatgtccggctgccaacatgaggtacaacctttgttgcctctggagcacagcttctctgtgctctcagaaaacacttctcagaagactttatttcattaatactggtctttctttaatcggcactaatttcttaggtccagctaaccagcaacaaatgttctagggatgtaaaggtttcactttaagtagttctggtgtcttgttagtcacagctacagctggttctttagccccagctggccagaaccagggtctaaatcacagtaacaaatggcccggatagtctttaatcttccctctgaaacttcacaagacaggcctccatctttattgctctcaacattcttttttttttttttaatattttttaaagatttatttatttactatatgtaagtacactgtagctgtcttcagacacaccagaagagagcatcagatctcagtatgggtggttgtgagccaccatgtggttgctgggatttgaactcaggacctccggaagagcagtcagtgctcttaaccactgagccatttctccatccctcccacccccgcccctctacattcttatcttacaggttccttcagaacatcccactgaggtgtcaacactcaatggttcttctagcccaaagttcttacatcctcccacaatccttcctaaaacatgtcaggtctcttacagcaataaccttctgtattagtcagggttccctagagacccagaacttaaggatagtctctatattgttagggaatttgttgatgacttatagtctgtagtccaactctccaacaattgtcagcagtagctgtgaatgggaagtccagggatctagcagttgctcagtcccacaaggcaatcaggcgaagaagagagagtaaatcttccttcttccaatgtccttatgttggtctctagcagaaggtgtggcccaaattaaaggtgtgtggctcacgcctggatctgggacttgctttgtcactatgcctcaagagctccatgccaagatccaggtcagaaacttgtatctcccagattaggatcacaggtgagtcttccaattctggattgtagttcaatccagatatagttaagttgactaccagaaacagccactacaatcaccccttgtcaacttgatacagataacatctcatgtccacatgaaacaataacaaagccaagaatacacctaacatgatatacttactatccgtcgtacaattgcaaacacatttgtaaatttacaatggggcaatgtcccttgagaacattcttttagtgtctcaacttaaataacaattgatgttaaagaaattggaagaaaaacaaatgtattctaaacaaaataagacagaagcatttacattacttgataatcctcatttctgcaactggttacatggccttagctggtatttataactaccttcctctactacccattctgtatttccttcacccttggcaaacacctcagcaggtcttggctcttttcctggaggattgacccataccttcattcctgatgggtctgcatcctttgtcaccttgcttggattaggctgttgtagtttcccattgactttaatcacagggcatggtagtactaagagacgccctaaggggtctcctacattccacacataatcctgcttacctccattgtggagaggcaatccaatttccccatggtaatctggatctatcacccctcctaacactgttattcctttcttagcctgttggtttaagggcattagaagcccaaaatgaccagggggaagtctgagctttcagttcaatggaatgtttgttgtggttcctggtatgagcactccctgctctggatcccaaacttctaggccagcagaacctagagttgtggggacaggatgcaaaacatttcctataggttcactgggagtgataatgagtggaactattcccttttccaccccttgattcctggacccatggatcctgactatgggggaaactctaccatatattggatgctgattcaaagcatatactgccttctgaagaactctgccccagccttccatgctgctgccacctaattggtgctgtaactgtgtcttcaaaaggccatttcatctttctgtcagaccagctcctttaggatgataggaaacatggtaagagctgtagcactttgctggctgtgaaatgagttccttggtcagaagcaatactgtgtggtataccatgacgatggataaggcattctgtgagtctgtggatggtggttttggcagaagcattacgtgcaggaaaggcaaatccataaccagaaaaagaatctattccagtaagaacaaaacattgtcctttccatgaagtaagtggtccaatgtagtcaacctgccaccaggttgctggctggtcacctcgaggaatggtgccatatctggggctcagtgttggtttctgctgttggtagatctggcattcagtagtagctgtaaccaggtcagccttggtgagtggaagtctgtgttgctgagcccaagcataacctccatctcgaccaccatggccactttgttcaagtggtcattaaacaacgacatggatggctggggaaagaggctgactgtccacagaacaggtcatcttatccacttgattattgaactcctcctcagttgaagtcaccttttggtgagcatttacatgggacacaaatatcttcacatcctttgcccatttggagagatttatccacatacttcttccccagatgtctttctcaacaattttccagatctttccaggtccttgaccatccagtcaatccattggctatagcccactatgctggtactaacttgtcttagggcttttaatgctgtgatgaaacatcttgacaaaagcaattggggagaaaagagtttatttgacctacactttccaatcatagccggtcattgaaggaaggcaggacaggaactgaagcagagcaggaacttgaaggcaggagctgatacagaggccatggagagatgctacttactggcttgcttcccatggcttggtcagcctgctttttcatagaaccaccagcccagggatggcaccacgtacaatgggctgggccctcccccgttgatcactaactgagaaaatgccctacagctagatatcatggaggtatttccgaatctgaggctgcctcctttcggatgactctaactcttgtcaagtcgacgcacagtacagcgagtaaggagagaaatccagacaggagtttttaaaatgggaagtaggtgaggtaagagcagcctggagctagccacttgttgcttcatatttgacaaagattactcaggagatgagcagagaggtaggctctatagaggatacacttctcggttccaagatgcaaactatacgaagacagttttctttttttgtttcacaaaaataatgttaaatgattagctaacaattaaaatgatagctaagttgggagtcccattaaagaaacagactcctcactggttgctacaaataatgtgttttccatactgcagtgaaagatttattttgaagttcagaacgttcctgcaaaatgatagtgaggcacagtggaaagaaaatctaaaaggatttgaatccatagtggttactttctagttccttatgatgctgctgagcaaggctgtctctctctctctcactctctcactggctcttagaggtttcaaatgcttcacctatggctgttgcttgtatgaccctctcatattctgtcactagtagtgagctgtgggcacacacctgcattgtaggggctgtgcatttgagaaagcctgtcactttgcccattgaattgggaaaccatgagtctttattctgattccctgatgagatttttatctgatggtactgtaggggaatccttaccactgaggacacgggctccaaggccaacaccgtccttgaatttaacagcagacagtaggagagtcaaaaacagatagagaagttttgtattatcttcagttttacagactccccccacccaatacttaatatagatttaaaccaaagtactctaactaaatactgaaagcacagttaagctctgaacaatacatacacttatatacccaacacatcagtcaagatagagattatcaccctagaaaattctttaaggatatattttatgtgtatgtgtgtattagtatctgaggagaagagggggttggatcctctggagctggagttacagctagtgaactgctcaatgtgggtgctgggaactgaattctggttctctggaagagcagcaaatgctcttaactccactgagtcagtctttccaggccttactcccaaattcttttaagccccatttcagtcactatcacacttagaaaaactctgtactgtcttacatcattgtaaatagtttaaaattaaaaaaaatattgtcagtggaattttacaatatttcttttgcatctggatatgcatgcatttacattcctgtgcatgcacagaggccatcctacccttttccccccaaactcccttgacacatgctctctcacccaatttgaagctggactagaggccagaaaacccaagtaaactctttgtctctgccccctcccacaatgaacttggtgaatagattaggctggctggcctgcaaaccccaggcatccatctgtcttggctgtcatttgtgggggaaagttaagcatgtaaatgtttctaatctgccaccaaggtattggcatttcatgttcttgcagtgattcaaagggtagacaagagtgatagagatacatggtagaaatgactagtgatactacttaaactgagttcaggtgtgtgacttgtctgctttattaagcagggatagggatctgagagtgattgtggcactcagaaggacacatttaattctagaggctttgagcccatgatcatcatgccaaggaacatggtggcaagaaggtactgcagtggatcagatcagtagctgagagcttacatgtgctctaggagcatgaggcagatagggagactgagaagtgggagtgctgtggggcccttttccaaacgaggtcacacctcccaacccttccttaacagttccatcaactggggaccaagtactcaggtccaagggactattgtcattcacaccaccacaagcactaaggacttactttgccctggtcatttggtagaaagttaccaacgtgatgtactgtctcatccaaacctgccctttgttccttctttccctcttctcccttcgcccctttcttttccacacccttccattttgttttcatttgtgtgttgggggtgggtgggtttggttgtggtttgtgtgtgtatgcatttgtatgtggggacagaaggcagttttggggtttcattctaccctgttcttgtcacacagtctcttactgagacttggggctaggctgtgcagaacttacagacaagcctgcctctgcctcacaacctcacaagcactggccttacaagtgcatgccaccatgcccagcttcctacatgagttctgggtaccaaactcagtttgactggcaagttctttaccagctgagctatgtgtctccgatccagtgtacattttgtatgaataggacattttcatgcataatgacagtatagatataaaagcaaagatgttggcatcaatatggcatttcccagacctcctttcagttaaccagttatctggttaatgtgtcttgtgagagattgactgcaagactttgtgttgcatttgttaggcttctagaatagttcattgatctcatgtgctactttcatccctttcaccacttggaggtgcttctgattctgtacagtagtaactcttgcctcatgttcccttagtagtggttctagttgtgccttactagtggttcagtatcagaagtgccagagaaatgatgctgcctttgtcttgctgcttagggattaggtccatcagcagggatgcacttttatttattgtcagtctgtctacatgagaataaaaacattcacatctttaaaacatctggctagttcaagattcaagtgcttgccttgcaaatctgacagtaagtttgtgcctcagaatccatagaagggtaggaaggcactgcttccacaaagttgtcctctgcccaccacgtatcatttgtagcatgcacatgcccacatgcatattatacccagacacgcagtaacaacaggaagaacacctgcatggcaatggttatccacagctcatggtaagtaagttagcgtggtccctgagttcctgacagtcagtgtctgatcagaagctgctctgtctcccaccctgtgttttgggtgctcattgaaacctctttttctctattgcactcaccccacaaacagtatcagctcttactaaagttattaaagcaagtaatagagattccattttttctttttaaacattatacattgtctttattacatcattagatcttatcatagatttctgctggcctggaatgtgccagaataattcttacacacttcttattaagaataaaaagtcatatttaaatcatctttttggggggaggttgcaactccctggtaattaattcagtgtttggtctttgaaatcaatcccttatcttttcataaaattttcaaactcaagaaatttaataaaataggtttagctagtttgagccaaaacgaagcttaatttttcagaatctcggcttagatgacttaaattaaataaaggccataagttgattgattaattttatttgtagcgtgacagtttattgatagtattgcatagttctgttacttagagtggtgccccagggaccaggaacagataaccagactcgatgtgattataggactgagcaaacagtgctgcctgcccacggagcctgtgcagtgcggctcttacatggctcctcctctcccaatctctgccttatacttttgagcctcgtgaatagaaactatttcacctgcccttatttcctgccacacaaataatgtgggtcacatggccaggtgggttagttggaaggtctcaccctcaagtgacaaggccaggccgagcttgtttgtcactgctgctgagttagaaaggctcctgtggtatgcaatttgaaaacttggcataatgcttatgcttccatatcaagtcaggcttctatttggccaggtcgggaataggctttttctcagttactctgtgacaggagatggagcacctgcacttgctggtccccttttttttgctctcaagccgtagcagtggtagtatcatccgtggcctcttagaatgcattgcttgtcaaaagcttatattcccattgtatccaagagcctcagctgatggaagaaacagctgccatattcttgatgctgtacagagacagacaataaatatgttacaggctagcttctgcacccttcatctttcaagtctagagcctagcccataggatggcatctatatgtaaggcaagcctttcttctttagttacatatttctggatgcaaccacagacttacctggagagatacatactttaatagtgatcctaaatccagtcaagttgacagtgatgatgattaattatctcactcttctggaatgcccattttatgtccgtgttaatatgtttcttagtcagtgttctgttgctatgaagatacaccatgatgatggcaactcttaaaggagaacatttcattgaggattgcctacagttcagagggttagtctattatcatggggaagtatggcagtggatagtagacagagtactatatgtagctttagctgagagttctatatctggattctcaggtatcagggacagggacgaggatggacacacacacacatacacacacacacacacggggtggggagagggagggtagacagacagacagacagtgatcctgacttaggcttctgaaacctgaaagcccacccccccaaacccctacctcttccagtgcacacttcctccaacaaggccatacctgctccagcaaggccacacctcctaatccttctcaagtagtgctactctgggatgaccaagcattcaaatataggtgcctgtaggggccattcttattcagaccactacagctatgtatcactgaggatcttttcagctctttttgtggttcttgattttttaccttaaagtttttacttcctgccatgggagataaagaatcaaaattactaaaaagcaaaatgagcaataataatttgaaataaaagtttttagttatagtattagtagtctgtactaaagatgacaagatttatttttttgtctattttgttttgagacagtatttattatgaagactaggctggtcttaaattcacaagtttctgagttctaggattacaggtatgagctattaaactatttgctaaagaggaaaaatatttcatgattttatgctttataaaaaactaaatataatttataaataaatttcagagtttagaaagcatttatctttgtggtgctgggatgacatagtaagaaaggacagtgcctttctgatatggcctcacatctagtgggggaaggccatacatataaatgtgggcttgtggatccataggcacttcttatgcagcaaggcttattctagtgcttctatttatttagtattatgggaagaggcagggcaaggggagaggggagcttagtgcaggggcttttactgattttagtggactagtgggagagtgtacgtatgtttgtgcaggtttgtatgctatgcagagtcctgaagttgatgctgggtgtcttcctctattgctctacaacgtatattttgcaacagggtctctcagaaaactgggtgctctagattggcaggctaacaaataccattgctcccctgcccagctctgggttcatagacttggattggaatgctgtagttttacatgggtgctggcgatctgacctgagatccttatacttgtgaagcaaatactttacccactgagccatattatgagcttacctgtgaccttttggcagttatttgtggacatgtacaggactcccccctcccaaaaaaactggacccaattggcatgcatgtgttaataagcaatgctttaccttcttgtttcagctccacatcataaacaagtgtccttttcatgtctatactgaaatacttgcccatatttgttttctttttgttgttgattatgttgtttaaaatgatccccagtcatacaaccaaattgccctctaatatttgtaagttccaaaatgctttgatactgccatgtgaaaaaaatactgcagtttagctaagtctcgtcccgacctgagttgtattatttgggcatgagttcagtgttgagtcaatactgtacattgaataagatgccttttggctggtaggtcaacagtgttgtggtctagatgcatttttcttcaagagcagtggcttgggatttactaattcagtgggtaaaccaatttcaccaaacattctggtcatgactaagagaactaactacatttaactaatggtggtgacctatagagattttaggtattgtgaatgttttattatccccactccgtctgaaactactgtttcagttacgtttaatatttccttgtagccttgcatgtgaaatcctattttactgtgagttctggtcagaaaggcctgacacaagcagaaacatgtcttagatttgctgttgtttgtttttttttaggggtctttgagacagggtttctctgtgtagccttagctgtcctagaatttagctttgtagaccaggttggcctcacactcaagagatctacctgctcctgcctcccaagtactaggattaaaggcgtgtaccacgactgcctggctagaatttgttttataatttattcttggtgagacttgggattaggtaaagaaacaattgtaaatacttctctccagaatattaatgtggatttgaaggaggttcttgttttagaataataatgtgtgaacactgcattgattctgatgttagaacaggcaatataagtgttagcacatactactgtcagggttttatcatgtgcttcctggagactaaggcacaagaagaacatgcagttacaagttcttaaacctgggctgtttttgaaggcagcatccttagcaagtgtaattcttttgagcctcggttgtctagtgtttagttagaaatatttccagtttgaaagttgtgcggttttgtcagtatcatttacattctcagcatgtccagttgtggaagaaagagttcagtattttgaaaattgtagtgttaaaaatacattagctgtcagatttcatttttctaaagtctagctaagtctcattagatattggtagaaacataagttttgtacctgataaatattgacatcatgtacttctgctatgaaatagtagagcatttaaaaatgttaaagttgaaaatttcaattaaaaaacaatagatactatatgtttgtgttttatataagtgttctgattcatacaaatatttctctaaagtgttcagaattagtttgtaaagcaaaatgtgtattcttgggagattattaacctttttgcatcaagtttcaagttctaggtgcttttttaaaaaatgtatgtaatgtgttgaaattattttaggaaaacttgaacatggtcaaatatcagcaaagaacgttctctgtaatacacagactgaacgctggtatagctgtcttgtatgaggctatgtgccagcctggcaaatacagaagtggatgcccacagtcatctataggatggatatacacagggcccccaatggagaagctagagaaagcacccgaggagctgaaggactctgcaaccctataggtggaacaacaatatgaactaaccagtacccccagagcttgtgtctttagctgcatatatagcagaagatggcctagttggccatcactgggaagagaggccccttggtattgcaaactttatatcccccagtacaggggaccgccagggccaagaagtgggagtgggtgggtaggggagcagggcgggggggagtatagggaactttcgggatagcatttgaaatttgaaatgtatataaagaaaatgtctaattaaaaaatacacagactgaaaaatgtaacatgaaatattagaccatatgctagcacacagaaaaaaacattttcagtatgtaagttttgagaaataagttgaaagcctccataaatacccaatcttcataatgcttttaaaatgcaacagcatgtcttggctacaaattctagctcatggactaggttcttgtaaattcattaactagttaattaatttatgtatgtgggtatttttgtttctatatatgtctgtatatacctgacctgaagaaggtgtcagatcctctgtaactggagttacagatggttgtgaacctccatacaggtgctaggaattgaacctgtgtcctttggaagaacagccagtgctcttaactgctgagccatctctccagccccactcatgggctcttgtatcttgtatactaaacatctggattttattctcattctccccaccccctttccatcagtgagcctcttctgtataaagttccctttcctattttcagatccttaggataggctaccacaagtggagttactgggttagagagtatggacttttgtgtgtttttacctacaatatttagatttgttgttaattgatttatattcattgtagaagataaaaattacagcgattgtgcttcttatctgaaggtaaggttttatggttcaggaagcttgggaaattgccaggataataaagctccttgaaggggacttatgccaacatccctgaaccttgctggtcactttataggtggctttgttgttgttgtttgtatatgtttaatagttagtgaatgatttatataggaattattttattggctaagttatatcttattgatccaagcaactaaaccaccaagcttttagagatttatgagaaaaggtagttaaaagtctttagtatgattttttttgaaaaaaattataacttacttaatttaaaacttatttattactaacgagcgcgtgaatctttaattatatacttagactataggcaaatgacacgaaaatagacagtaacaaagattgtacaaatcttgtttatcccccttttcctcctacagaatgtgaaaacctttgcatcttctgatagtctagccaaggtccaagaagtagcaagctggcttttggaaatgaatcaggaactgctctctgtgggcagcaaaagacgacgaactggaggctctctgagagggaatgcttcctcaagccaggttgatgagggacagatgaatcgcgtggttgaggaggatccacagcagcaagcgagacatcaagaggaggagcacactgcgcggaatggtgaacttgtgggtgcaaaccctaggcctggagaccagaacgatacccagcaaggacaagtggaggaaaataataaccgctttatttcagtagatgaggactcttcgggaaatcaggaagagcaagaggaagatgaagagcatgctggggaacaggaggaggaagaggaggaagaggaagaggaggaggagatggaccaggagagtgatgattttgatccgtctgatgacagtagcagagaagatgaacatacgcacaatagcaatgtcacaaactgcagtagtgtctcggacctgcccgctcaccagctctcctctccattctatacaaagacaacaaaagtgagtacctttagtccccttccagctggtatgctacctcgctagttcagttatagtgaagtcatccttgcaatcttcacatgcagaacaactttagtaagtcagttttagtttgttaaaggctttagtgttagaaatacagatactaataagaagtcaacttaattgcaaaactagggggtgcggattgatgttgtttaccaccaaaaaaaaaaaaatcaccagcttcacctttgctgccagagtggtttgggtgagtgtgcagggttggtgtgaaatatgatgtgtgtaaattaagtaatgaagaatcttatttgcagagtagctaatgtagtcactgtctgtcgtttcaccagcgttaactctaatgggtaagttagtggaagtcacagcaaatagttctgcgttgttgttcagaagttaaccgtggctcatggatgtctaagtagactagctttcagttaacctgttctagggattgcatggagtgacaggtcacttgttctacacacataaaaacaaacagtccacatcagacatccctttactcatttgctgatttttaaatttaacaatgaatcattggatttagtctgtaaatacctgtaaatttaacagtatctctttcaatatggtttgactttaactatagtatctaatacagttggcttaaaattcaatttgctgaatcagttttgtttttaggttgtaaaacaaactgtctcagagaacgtttgtacctctaatctccagagcctggtcacgttgttttatttacttgctggcatggcttaggaaggaagtggcatcaggtcatggctctctcccatttaactttctcaaaatggagggttttggtctgggctgattcttagtgttttgccgttctttttatttccgtcatagcagtttagaaaatcctctagcactctggagggcagggcaggaagattatgaattcaaggcaagacattctctcaattgcttaaaagacgttttaaaagttacctgtagctgtattaagacttcacatatcgaaatctattaataagaagaaatttatactaattcagtattttattttggaggcctttacatctttgatcattataaaacaacagaattgtacagtatcctaggctgcagtttcatttcagtccgcaacgtggggccttagagaagagtagggctgagatgggaatcctactggagaggcaggtgtggcagcctcatcacttaaggcaggaagattgttgtagatttcaggctggcctgggctgtatagtgggtgtgagtctgggatggactgttcagaagacttttatctttgaaaggaagaaaacgtcttccacgttctcagtttgctttaaaaatgtccctgtagaagtctccagcaatcctgaccgcagctccacttagtgtgtcattctgcagcatttctgcattaatgagaaaaggcgtgtgttctctgttgcattggtttactctacagagcacaatggagactgtccagactaagggcagtttcctgcccttggtcattgagctttccttttttgtgagtgtagagaagacggacagcagttttgacctaactgtgaagtgggcacccttaagtgctgagtataacagtggccagtggttcaaggcagacttgtactagttcttaaaatttctgaactcttgacaggttttttgaggtgctttctgtccaggtgactgacagacccatttggactaactcttagttatagggaacagtctctgctctttataagaaagccatgtttcctcttggggccattaagttttgttcttaagggaatatgccataagcagtatagagtatattagatactagataaacagatcgggaattctaatattaagtgtcaaggctaattgtagtcaataattgctgcttattaaatacaaactgtttacacgttcctgaatagataagtaaacaggttatagggagcccaatggttcactccacaccacactttctttctcatgtttagatttttttctatttttcttttcttgagaatagtttttttttaaatacaatatattctgattatgcttccccccccaacttctccaagatccttctcacctccttcctgtctgtacaccccaccctttctgtcatgtaaagaaaaataatagaccatgataaaacaaatccaaatgaataagaatagacataactggcaaacaaacagaagcaaaagagccaaagcatgagtaacacatacagatgtagagacataaacgcttatacacataggagtccataaaaacaaagacctggaagccatagtatatatgcatagggtctgtagggttaaaaagatgaagctttgacaaaacattgtgagacagagaacctccatggatgccatgtagtttgtttgtgttgatcatctactgctgggcactggaccaacccttaagagtggtttgtttcctcagtgagactctgttggaacaaagtaattatcaattgcaaatagcttctaggtttgcaatggggctctgtctacttttcctctcagtgctgggaccccatctggcacagacctgtgcatgttctgagtaacaagactttctaagaaccctttctctttccgtgtgctatgctgccccctatggtaggatgtaacacacagtacagcagagtttccaagtttgataatgaaccccctgagaactcagctagctgcttttcttgtggtgttaacctgactttattgaagatatgaggccacaagacctaagttgtaagaggaaggaaggatcaggaaaaaaggaattggaggctctggagtctattctatgctagctgtttttctctgtgtgacaagtcttcaccaagttctagagtcagtttgctttgccagttatgagtgtttgtctttgcaggtttaactgtgccagcttactatatctttttttttccctaaattattctctcaagagattctcagatcatattgaatctttggcatgtgataatttaaaatacagattattaacgtttgcttctgctttatctttatcagctcactttcagctaacaggaaaagcaaatggagctacagaggtcaggtctttatcctcttgatttttttcgtgaaaacaagcatgtttgagaagtcatttctgtagtaactccattctttatctctaattattagattcttcctagtgatggtttttatccatttgcttctaggaagaactccatatacacaggcatacacatgaatacatatgcaaagtagatagagaatacattgtgtctttgagctctaaacagtagaggatgagcagggtctagttgcaagtgcctatagtcatggcgaccagagagaagggctttagaccataaacttaaagtaaacaggtttgtgggggctctactggagctggaactaccagaaaactctatataataatgtgaccaacagctgaaacttaaagggaagctaggttatactctagagaatgatgttctcctcagaaagtccatggcttagttctgtccataactctaaactcccaagttgtattaggtctaggtatgtccagagagcagttgtcttatataacatttgctattatgcacagccctgtgagtggctgtgtcctttccttgtaaatgagaagaaatggaaacatggtgaggtctgcttaatttgattcagaatctgggggagatgatggcagggaaaaggcaggctgtgaaggatgaggtaggttccctaaacacggggaactgtagcttgtggtgcctgaacgtggactcttgactctttcactgactatacgaccatgtgtgatagatagccttattgaacttttctttatctgtacaatgattacctttttttagaagtttttcaaggattaagtaaaatcaaatatatgaagtaggtagcatgatgcctgagaaacgaggctagtgtgttcccataggccggtgacctggctttgtatctctcccttaaaatacaattcattctctataacacgtatatctttttgtgattttcagtgttcagtcattatgtcactgctaacatacaaaagatatggagaaataagcagaaaataggttaatactttaacaaaatgcatgcaagtagttgaacttaataaactgggcttcagtaaaaagaatataagctctaatcaaagtgttgccagatcttgtcagtcatcaggctataagctattacttggagtagaatttgtcagtgcatctgtactttttccaaactttaactgtaaagtttctgcttacatgctctgtaatccttgcatttgattgataaagtcaatgtcaaattgtggtaaatttctaattaatttctcttaatacagtttttaaaatatttaacattgtttgaaactatatttgctcaatatatatatgtgaatcagattatttttaatctatattccattcactgaaagtagtgaaaagaatatcttaattaagagctttctctgtaaactaaagatattttccctatagaacatgttaccttagcacttgagaagcagaggcaggcatttctctaggccagccatagctacttgatgagaccgtgtctcaaaaacaaaaacagaatggggaattttaaaacttgagtacaaatgatatcattgagtacaaatgataaacttctaaaatatgtattagtaccaagcagagttattccaagatgtttttgaggcagatcgctttatatgtataattacaagggcagtctttttaatgttattttgttggtggtaaggaagatggtatgtagaataagcatgcagtcttcaggatgaatttcattttagttttgatatataacttcagtattcttaagtatcatagcaggcatttgttagtatcagaaaatatagatgtaactgctaggaaccacttattttacctaggcttttgtgtaacttttctaatgcataatagagggttagagaaaaaggttttcttcccaagaactggcacatgcccttaaaggagggagagaagtgagactagaaagtgtcactcacttgtgtagtgggaagggtgggtgctttgctgtctggccttgatcagattccacatctcagacacacttggagctctggcaaattctagctaggtcagctgactccattgctttctgagtttggctagcatggggctctatttcatttatgtttgtgcgtacttcatgcatgtgggtactttagaacttagaacaggtactagatcctctgaagctggagttaagacagcccttggtgcctggtggggattctgggaatagtactcaggatctttggcagaccagaaggtgctttaactcctgagccatttcttaagctgaaaagttttttagtatgtagtttcctgaacaaattctttagtgtggccctgaaagcttctggctctccatgtgtctgtcacatttctattgttgtaaagagatgccataaccaaggcaacttatgaatgaaagcattcagttggggacttgcttacatcatggtagagagtgtggtggcaggcagacagacaggcatagcactgagcaagtagctgagagctcacatctgaccctcaagcacaaaagactaggtctggtggtgtgtgcattgaaccctcaccccacccccacatcccacgattaatttctctaacaagggcacacctcctaatctttctcaaacagttttataaaacaggaccaaacatacaaatatatgaactagtggggctcattctcgttccaagtattataccatgaaacataaataactttcattgagggagggtggaggggacacagtttgagaatcctgtttttaaaataattttgaggtgcccaatgctttagcaggaaggcaatagtactctccttttccttccctgctcccgactctccatttatatagacctgttactataagttattctgtaatggaaactgacaaggagactgggctcatcagctccaaatgtaaggtcatcactcagctttatgtttaacagttctttatgttaacctctgtagtgagaattttggtttattaaaaaaaaattcaattttgttgtgtgactatgttttttgttttaatcccaggtgtgggatcagaggctgcttcacagcaggtgattatgatttgccttgtgcactaccaggggcgtgttttttttttttttttttttaccagctacagatagtttaattctggggactctagagaaggtataaatgccagatccccaagaaggctgctgctccttctcctgctcctggttgatattgttgtagtttgaagagaagaagttggagaaatcttgctgatgaaaattggacttgccccccaaatccagcatccctagtcaccaggaagtagtctattttgttggctgaaagaggtcttcacccctctcccctctaaccttttctcctatatagtgttcagggttggaaggggttagggtggaataagggttggaagagaggtagagataaaagaacccaataaaatagtaaaaaacaaaatagcaccaactgacctctttctggtcattgtgtttccatgtagagtactgacatctctgtagtcttcagtagaagactgctgttataaggaatgtatttggtgtattctctgaatcaacgtcctgtttttggtttgtttgttttcgttgttgttttccttgtttttttttttttttttcttctggataagttgctcttgaaatggcatgcccttttttcagcatgctaatgctgaggggcagttgaccacaccatcttttgtccttattctggtcctcagcagtgcagtggaagatggactacgtaatggtgatagccatagtaatgattgatatgtagccatgtgtgtggcacgttgcttttgtcccagctactttgtattttcttcccaaggatcatctgagctcctaagttcaataccaggctggggaacataggaaaaaccctgtctcagttaaacaagcaaataaggataagtcatttattgaacacatttctggggtaccaatagattccttatgtggatgtccttgctgagtctcgtcgttgccagcctcgtgtctgagatgacttatgagcaggaattcagttgtcctcaggttggtagcagagatggtattgagctgagatttcacttcagcccaggttgttctccctgaaatacccagtgaagcagtatttccaactttgggatctgcagactatggaatgaccataaacctaacatctgctttcaggtcctcagtgtcaccccactgtggcaagtttaaatacacagtttcatagcgcaagcctgaggatcccctgggtccctgaattactgtgttggtgacatagttaatttgatagcctgtctaaacaattgattatttaaacaattggcctcttccttacccaactgaaattatgctcattagcataaattcttcctacttactttaatctcatgtttcatcggtgcgctcaccctggcccttcagtcctctaaccgtagaggaagaaatggcgtacactcaaaaggaagtctgtccttgagttaacaactgattctccagacttgtgaaatggcttttttgggacagtggatggggtggggctttcctacattagccattctttgtaacttttaaaaattaacaacctttttattttcaaggtaatagcaaaattgagtggtaacacagaggctcctgtatgcttcctgtctccatccacacatatcataccccacaataagcccctcaccagaatggtagatgctttacaaatgataaggcagaggccacacttgcatcgggtttcactcctggtgttatgtattctgtgggtttgaacaaatgtgtaatgatgtatttaccagtgtggtattatataaaatagtttccattgtccattgtcctagaaatcctcaagatcagcctattcagtctccatccacccaaccccaggctcaccagtctcttcactatttccacaacattcctatttctcaagtgccatataattggaatcatatagtttgtagtcctcttagcttgcctttttagtaccattaagtttctttagtgtattttcatggcctgaaagttcactttttactgttgaataatattttgttgtctgtttatctatccatccacctaatggggaatattttggtttcctagacttcggcaaccgtgagctgcagacaaggagtagtgtagttgtatatttcctttcgaaaaggaaacctatttgccagggtgtcttctcttgcctgtgctgtttttgcgttcttctcactgtcagcgactgctctgttcttcctggggcttggcatcctcagggctattggcttgggctgcttgggctgtgttgctgtccactgtgggcatgtgcacgtctccagtcgggtacatgcatatgtgcatcttcagactgttctcttttcttttcttttcttttctttttccttttccttttccttttccttttctttttccttttccttttccttttccctttccttttccttttcctttttcctttttcctttccttttttctttcctttttcctttcctttttcctttcctttttcctttcccttttcctttcccttttcctttcccttttccttttcctttcccttttccttttccttttcctttcctttttcctttcctttttcctttcctttttcctttcctttttcctttcctttttcctttcctttttcctttccttttttctggctcttttcagtttattgcatgaaggagttacactagtccaagttaaaagcagaccccaaatgattacattatacaagctgtgaggtttttaaacttgtgacaagggacagaagggaaattctactcattgcaaggaaatcctcgcttaagcttcagagagccacaagcacttaaaacccatgagccttcagctgatcgtccttagccagcccagtctctatcaggacctggcacatgttcttgcgctggtcaccctgtagctgaattacttctccatattctggatgctcaattacagtaccattgcaggcaaatttcttcttaaacaccttcactagtttctttttatcgtaatcatcagcgatcccttggacagtggtaagggtcttcctgccctttctctgttgagttcttatatggatataatcctcagagccagcaggatgcaggtcatcacccttacttacttgcatcagcaaaggggtcgaaagagtggaggttctggatagcagacatacagtacgattccttttcctcagtggaaatggcctgcgggagaaggcgggtggcggggcgggggggggggggggggcggagcgtcgggaagcgagggggctcgagggggaggcagctgagtccttggcggcggctcagtgactggggctagactgttttcttagcaatgttgtttttcaagagttttatggattttttaaaaataaaagttatttgttaatattttacaagtatttttggcttgtttgtggtgtgtcttcttattctagtcatagtgtatttcagagaacctgcttaattttaatgatgtgcatcttctctattacttcattcatagacaatgcctttcatgttgtaactacagtcaaggttatctgcttaggtttttttcttgcgtgtttgcagtctgctttaagataatttttgaaaagagtgtaaggtctctgagtgatgatgttgatgctgatgtagaggttcaggtgccgtggtaccatctacaggagactaacttttctctattatattttctttgtatctctgtcaaagatctgttgactgcatttatgttggtccatttctgggtcctcagtcctgttgcgctatctgttccccaattgctctcttgatggctgtaactttgtgaaaagacttcaggataggttagtgtcaatctttccaggttgttcagtattatatgggccctttgaagtctctaccgtaacatttaaatgttggagcatttgttagtgtctgcagagtaactgctgggattttgtttggaacagtgttgagcatttttgcttctgctctgccttcccgtgtttagaactttagaattcctttatctgccttcccgtgtttagaactttagaattcctttattatattttcagcatatggatagtaactcaaaagacagtgcaataatgaaaacctacaaaagagataagaatgttcagttttaaagacgtctacaattaatagatttagcagtgtccttagtaagttggagaaagtgatttttgcagtgaaacatgtaatcattaaagtttcttcattgaacactagagggccatatagtaccttgttaatggtgctgttttcatgtatttaaatatcttagtgtctgcctattatttcttgctccaatttggaataagtattaagggaagttgtttatattgacatataatatttttgtattttttaagtcaagaaactagcatatttcttattttgacacatagtaacaaaaatattcagtgcatcttgtacatgtgtataaactcactctctcctcaaggatgagtacattttgaaaagtccttaagaattagcatataatcttcagtcagcagacctcccatgaccagtgattgccctgggcttcctcagcatatgtgcaaaaagagcaggtccctggcgttagcactacagtaggctgaggacgcctcgtcatcttgaagtaaagaatgcttgaccagttaattttcttgacttttcttagtttggtagtggggttgaaccataatgtttaggccctaacaaagtagagtgactgtgtgaagaggaacaggaacagtttgtttagacttttggaatatggatgactacttgtgaaagttcagagtctgatcagaatcgtgctgaaattgtcaacttcttgcctctgtttatttgatagaatgcttctcattcttaatttttgtattctgggggacgtaggacattctcactagtcagtgttcatgctgtttttaagaactggtcaaggatggtgaacacagtgggcgtgccttagctacactgtgaacgtggggaagggatgggttggactctgagactgccaagggtaaaccaccacatccctgagcctctgcacctgagctgtcattattaagccacttttaggttgacaatttaaattttctgggcaggttagtccctgaccatacatggttgttagagtcgatatggataaacagtggtgtctcgtggtgtggttacggctggtgtgctctcaccacacactcactgctctgtaccgctgctgttcttctgcagaatacacacatgcttagttctggcagagtcatgtgagcagagcttaagtgactaaacaccttttgctccaggaatgaaattagttacctcatctattatgttggtgaaagttaaattcttaaatggctaataacgcaatccaaacgagaagagggatcggtatcccgtaaagcctttctatggctctgttttccagagcgccaccttcatttacatcatacacagagaaaagtagctgttctcactcattgcaaaatcaaagcagttttcatccaacaaataggattaatagcagttggatagaaacatagcattgagtgccacaactgtattatatggtctgttttcattttcaattagattattttcagatgttaaactgccaggtgatctaatttctttatcgacatgattatccttgtttttattcttttgtgtgtgagtgttttgcctatggaccacatgtgtgcctagtgcctgaagaggccagaagtcagtgtcatatttcctgaaactggaattgttgcttatcatgaggcatcatgtgtgtgttgggaattgagcccaggtcctctagaaggacaatcagtgctcttaaccactaagccatctctccagcccgcatgggatcccatttaaaggatgtatcccagcttgcacaaggaatctgaggtggtctgtatttcggagtagcatggtaaaatggagtatatgtgagaaccagcgcaatgggaataatgcagtagaagtgaggctgaggccagagaggacgaaaggaacgatctacagtgagttggggcaagctggtaatagcacgtaggccgggcatttctgtcttagggttttttactgctgtgaccagacaccatgaccaaggcaattcttttattattattattattattattattattattattattattattattttaaagatttatttattattatatgtaagtacaccgtagcttacttcagacataccagaagagggcatcagacctcattactggtggttgtgattcaccatgtggttgctgggatttgaactcaggacctctggaagaacagtcagtgagtgctcttacccactgagccatctcaccatccccaggctgggcattcctgaagttgaatcccagtattcttcagagtgtgaccctacaccacctcaaacatctggagtgctaattaggaatgtaggtcggccaagtctacctcagaactacttagccaaactctagagctggagtatgcatttaagggagctgtcctaacattcgtagcacactggttgtccatagagtggtgccataaaactgaggacagtgtacacagccctctcaatgaagaccaaatattctgtgtctgttctctctgcatgtctttctctcttactgggtttcatgagtaccttagcagattgtacccttggatgtataataagtgcattaaggaaacactgaactttacagttagatagctagtcaagaccaaactgttaactctttgatgatattaaggatgctggtagtacctgtcacatagtatcactgagagtgtttattgaagcctgaaaggtagccaggacgagtcctgagagtcttcagcagctattagctgattgtatttcataatgtcaattatttcaaagttacaagtttgtgctaaagctagcttgctgggatgtcatgcaaagcattgttggtttctaagaggtcatctggtgcattgaaactgttctgacttctacctaaatgattatggcatttaacttggagttgaactgtaggttatttaagtttagttagtactaaccatgaagtactcattttgtaattaactgcttaatgctgaaactagaaagagttggaacttaaaatattaataaactgggggccacttgggaatgctgagaagagatgaaaacctcggttgtcttcttgctatgctatggtgctgtggactcagtgactgcagtaactggaaggaggatccattcgatactcctgcaggttctgtccctgctgcgtgcgtgctgcgtgcgtgctgcgtgcgtgctggagagaatgcccacatgttcacactcactttctttctttttacctgtctactactgtgtggtcagtggagtattcacgaagcaaagttgaaagtatgatgacagaaaatttaaaagtgagctaattttttaaatcttacattaaagtaactcaggtgaaatccaagccatacattttctatagttttatattttattttgaaaatagccaaagtcttgctaaattggttcatttttagtagttatcaaagtgggaaagtgttaccaaataatctccaccctcagaatagcaactaatctttccatcttggatcaccttttgcccttttcaccgtggttaaaaattgaaattaacactctttggaaagactacatagatgccatatggtagtgaggactgtgtaggtgccatttcacagtgaaggctttgtaggcactgtatcacagggaagctgtatactcggagccctttatttgtatgtatactttgtcatcccccggcacattgaacactcagcctgtacaagtcagagtgcagcctgaggtgaagccgtgggtctgagtcagcggaggctggttagtcgccttcccatcatgcagtcttaggctttacccagattgctcttctacctgagctgccctgagctttattctatgactgcagatcacttggaaacagcacagcagtgtgaaaagcaagctccacttttttttttttttttttaatgacagcctaaatggatgttcctttaaacagaaatgtctgcccattttttttaagtgaagagaatgtagcgtacacccttcagagtaagcaatattagcgtcctactgcaaaaaggaatcttttcttaaagacagggatactatagtgatcatctttgaaaatgtatatgctgtaggatgttgtaatatctatggtctccttcctgcttcctttttcttacgtagaggctagagataaatagctggtgtcttaattactctccatctcaggcctctcactgtacccgaggcttgctagtctggctagccagcaagctttggggaccctttcttctccatttgggcactagcattgtagatgcttgagcccactgagcatgcaagtgtaagcatgggagttgggtgttgaactcaaggtccccatgtttacatagcaagcatttacccttatgtctctatctccacagcctctcactcacttcctcttaactctcaagatcttcctacacaagcacatttgggattaaagaagttgagttaatttatcttttttgatagctaattagtgttttatttattgaagaaatgaaaactcagacatctgctataaaattaatgttgaaatttatgagactgtcagtttatctaggaatgaaagtctaaattattttctatagtttgattgtatgtttaaagtattttatttagagtaaatattttttgtacttaatttttttttatttttattttaaaggtgtgtacgcacatatccttccctgcagggtccccagaagcacagagtagtgtgatttcctggcctgcaaacgatttgtaagacttttgcaggagaaatagctgaaaggttgatgagtgagttgtgctaaaacttgcttataaaagtaaagggcagactttaattgtttattttgaatactgtggaagatgaagttgttggaggctctttgtcgtcacatggggatggaattcttgcatgattgaaatacctgtaattacaatgacatttttgccatagttgacttaaaggtatagctatattaatcaccagctgaattcttggtgagcagctgtgtaaggccagtttaacagctggggtatcccggcataagcatgagagttaaacttagtcacgaagtaaaatttgttacagacacagaggactctatcctgggcactgctgagacacttagaaaacgtaatctgaaagttggactttagtttataatcgcaaatctttctttaaatgtctgataaaagaatgagaagtttgttttgaagtaaatagttcatagacacaaatattggattaaaattcaaagcttacattagaaaaattaaatctatcatctatccactttgtatgttattgcctaggtcattatggtggataggaaaagaaaataaaaatatatgattttttgtagtgttaattttttgaatttcatcttttggtcaatttttaaaacttttcattacatctataccttctttccagttaaaatattttcattgtatacaactcaactacactacctaacacattgccacgattgcctgttttgaaagagtatccatgctgggcagtggtggcgcacccctttaatcccagcacttgggaggcagaggcaggcagatttctgagttggagcccagcctggtctacaaagtgagttccaggacagccagggctacacagagaaaccctgtctcgaaacaaaaaagaaaaaaaaatgaaaaccaaacaaaaagaaacgtgtagaatatacactactggattgaaacagtgagttttgtttagatgtattctattttaaagtttctataaatcacatttatttacttatttatctatttatttatgagggggagggacatatacatgaaggtcataggacaacctttaagagttggttctttgctaccatctgagtcttagggatcaaacccaggtcgtcaggcttggcaacaagcacctttttttttttttttttttcattaaaccatgttgctggcccttggatatgttttgtaaagctaactttactaactcgtgataaagaaggctcacaaatcgatagtccagacacgcacatgagactaagaaagctacagtatacactccagactgtaaagttttcaccgtcagtcttctgtcccatccttctctttagggaatctgaagtgtgggataagcagccagcctaaagttccttgtttcatggtggcactgaccctagaatttaaaattcacctgataatctatgtgctgtgcagttgcctgtcagctttggataatttagcctagagcggtggttcccaactttactaatgctgggaccctttaaaatagttcctttaactatagtttaatatagttgtactgtggtaatcataaaattattcctgttgctacttcacaactatcattttactactgttacagaacataatgtaaatttataatggtcttaggcaacccctgtgggtttgtgaatttgtagctgctaggtctatttgtagaatgcccgtatacattcggtactgtttcagaatccaagctactgtctgcttatgtgcttagtgcttttgggttgttctttgtgcttagatatcgaaacttaacctaggaagttcaagaatacattgtagtattgctagtacactagtaatggtgggatgccctagaaactgtaacttgaaattgtaaacttcctgacaagtactctgagcatttaaatgtgtcgaacttctctggaaatcattttggactggaccttagctcaccctccctcatttttaagacagggtctcactgtgtagttctggctatcctggaatacagtgtgtagaccagactggcctccaactcaaacagtccctcctgcttctgtcttctatgtgccaccactgcttagcctggactttaaagtttataaacacagttcttccatttgatttcgttttatgaatactaaaggtataggggccacactgtatagttaaaatttaattttatgctgaattgttttttctatagttgaggtattattaatacaagaagacagtttattggtttgtgactgttccctgtttgtttcagtttctccatttttatgcataaatttcttaacgggcaatagtgtaagagtcctttctctttggcactaggctatgaaagaccgaaaattgatttagcacaggttgacaatattctatatataatgttctatattaaaagaatagttattctgacttatttaactttcagtagtgctattaaaagtatctagaattttctgtttcaccaagaacacagtggtgttaacaaaagtggctttaagttcacagaagcactgaataatactgagatccaatatttttcccattggaaaaaatgtgaaaacttatacaaaatcagtttcttaaggctggtgcagtgtcactttgcatgtagaagctgaggcaagaggattcttggagggaagactgcttgaggcctcgaatttgaggctagtctggacactgtaaggaaactagcttactgtgctaatagttgaggagccatgcatactgtacacaatgcccttcatggagtgtggttttcttgagtagctaatcacgtgaaccaggctcttgcttaatgtccgtggttgtgaggctctacacccactgtccagctcgtgcttcgttcatcagggctttcttggatatttgaagggtttgaaatttttcacatttccattctagattgacaacatggctttagaataagagttccacaaagtacagacacttggaagttagtagcttgcactgatcatcactgaagagctactttatatctttatccttataaaggcttggctggacacttcatttagtgttaggtttgctggagttgcagtgctcatattatccagttgagggcagctacttaccaatttaaaagagtttagaatgctaccacttggtgcttctgaaaatgcagtttctgatatacaacagtgctcactttctctcctgtgttttttattgtcacaaataatcatgggaacatggaaagctcctttttccttcgtaataattgtttagctctttaagaactgaaggagaaagccttatgttcacttaaagttcttcctgtcccagctcctttagatcccgtctttcttccccgcttacatcagacttcaaaagtcgcatagattggcttatctgggaacctggatcttaacagaacatacacataaacaaggcggctttacaggtctctgagacttttatgtgtaaatgtgagaacagacagtgtttattgtgctgtttaattaatcagtgggctgagtaaaaattaaattgcaactggcctgtttggtatgttttttatgcaaaataaattatattctcacagaatatacggctgtctttcatttttgtttttgtttaagcacagagatatcatttagtttaaatgatatcttccctatagttctgaaaggaattagaagtgctaattgttttcagagattatcactgaaataaattattttaagaataaagatctcgttctgtataaaattatattataatattattagcaaaatagatagtatttatatctggggatagatatattagcaaaaaggttattctaagaagacgggcagaatatgagtgagggttataattatgtgaacaaaatatcagtcttcctctagagagtaaagtgagtgtcaaaggacacacacagactgggaggtggtgacatttcttctggagaggctggtgggaccatagacatgtttctcttaactatcagttattaccttcccccacccccccgcccctggccctggccataataatttatagatttccagtgtccttatgctgtcttttctctggcctacccctgggtctttgttgggaatgatgttgtgcatgcactgccatgcatctcttagagcatgtccttgtttagaagtgacatgtgactgtgatcatggtccttcataaattattttataagaaaataagtgtatgtgtgtatctgaatgtgaatgtatgtggatgtctgtctgcatactcccatgcagagacaggaagacactgatgacttgtgcaatcaccccttgccttatttccttgagatacaagacagtctcttggagctaagtagaggctaccaatccccagggattctcctgtctcagttccctgtacaacagttactgactcacgggaccacacccagctttttttgtgggtgtgtgcaaagcttcatactcaggtcctcaggtttgaacagcaagagccctgaagtccactgagccaagtctccagtcccagtgttaaggactgttgttatattctactatcatttactttttaaacttgttactgtggttgagcaggtataacataaaaagtaatgtttttccttgttcacgtatgttcagtggcattaagtacactcagaatattgcataacaattaccaccttccctctccagaactctcccatttttttcccctgaaactctgtagcagttaaacatacttatctccacacccttgtgctttctgtctctgaactgaactgtccccacacactgcttaagtgtttgattataagtggagtcatgccacacctgtctctttgtaatgcacttgttttacttagtatgatagcctcaactgtcaatgatgtgtcaaggctggaattttcttcttgaaggcaaaacagtattcaactctgtgtgtgtagcacttagcttacttagattatttccatgttgtgattgttgtgaataatgttgctacactcatggtacttctagtcaagttcctgcttttaaatcgtgtgtatgtccatgtccatgtccagaagtggaaatttggagtgaccccttgactgtgggcagtgtttggggagctgccataccacttcccatcgcagttacagtgttttccatcatattccagttcccctacatactgccagcaagtttgcactttaagctacatttcaaatttgatattagagagaaagacctggtttaagaacataacatccggatcgggctaggaataattcttgtgtacaaactgtcttttctgctcattagtctacttacgatagagtaacattgagctttccaaagtgggaggggctgggggaagaagccagacaaggtggctcaccctgtaattctagcacttgggagttagaggcaggagaatcaccagttccaggtcatttctggctgcaggatcgaggtcgacctgagtggttcgtttaaggaaatatttaagattacactgcaactaatagtttgagaatactttcaggtagagtcgttgcctctcaagtttaccttgggcgttagggagcaatggaaagttctttaaacgtagatgttatagcccagtcctttgcgttttaggaagggttgtctaattgatatgttggaggggttaagcacagagaatccccacctaccatccaacatttcagtcacctctgtgtcagagtactcggtgacagggacacactgccatgggtttcttagagcatgtttctgctgagaagtgacatcgtactgtgatcactgcctatcatcgttcattttataaataataaacaatttatgtgccttcagtcagtcatgtggctattataatggactcaggaactgtttcagaactacagaggaactgagattgataatttctagtaagataatttgaggttattaacagcgagagagcggtagggtatgtttgctgtcttggtgagcaggagactggtctgtaaggtcagtcacttgatgcagaggaggctgaacaagcaagctgttttcagcatgcatcaacactcctgaagtttgaatagactcaaagcattcttcagacacttatggtgacgtttgagaaaggaagacttaagagaacatagagttgtgtgtgtgttgaattttgaaaaggaactcttactttccactttctagggagcaccttggttatttagttggaacgctcccaggaccccgcagcccagccctgctttgctcctgtctgcactgctcacttgctgtgggagctcaccagctgtttgactggcaggagcaggagtgctcacttcaggagtttataattcctggctattctaggacagtttgcacttcaccaagcctcagacaggtcaggacactcgcaaagagaaggttggaagacaaaaacggcaacaggccttggattctcagccttttaaaactactagtcattttttcagtttggttgtgagtgttttagtaatgtgcctgtagtacatattgagagtgttcatcaaccaagaggagtttaaaatgtcaaaacctggaaaagctcctgtgaaccatggcttggttcctgttgatctcaaaagtgcaaaagaccctctgccacatcagactgtgatgaagatatttagcattagcattattgcccagggtctgcctttttgtcgaagacgggtaagtctgtaacatttaaagcacgactgtttattaagtgctgtttctcttcctgtaaagcactgcttccgatcgcctgtgtgttgtctgtgaggagagctaacgatgctttgtgtggctagggaaaacttacacagcaagagccgggcaggggcaccctctctgcctttcgtctcctctgtttttcatgagattagaatcctgtaaacttatttcaaactggtaaatgtgaaaacggtatgtcttgaaatatgctgataaatgttagaaagttgtgtagcttttattttgctgggaggccaaactgctgttggtatgttccttacatgtgggaacgcagcatctagtgcaaagaacactccaggcaggtctgtaggcttcttgggcccgtaaagctgggctatttttacaaactctttcatgaattaaaacttcatgggttagaagagatgtcagttgttttctgaaacagaagcggcacatggttgggtgatatcccgaatgtcttggactttgcagcactcgtgttactgtataatcagcataaggtccaagggtgtttttcagtatgacgcactggggctgtagtattagcaatatgtgtttcttctgacctagctgctgttgccattttgaattatttactagagtcatgtatttatacatatgaaatatgtttatggtcctgaacacagtaggcccaaacacataagtttgtttgtgtgtctatgtctgtctgtctgtttctttggaagtaaatgagaacttgtttaatcaatgttttctgttcttacatctcttatagttactttaattttttaactgcaggggacaaggagaataatccacatcactagaaaacacttttgtctgtatagtggcatttactgagatataggaaagctttgtaatttttttctaattagcttagttccttttgagtgatttcaacttctatcttaataagtggcagccatgttggaaaatgaagttaaaacaatgtcttctaaattttgaaacaagaacccaaagtgctctcttgtcctatttcttctcttcttccttttctatttgttctgtcttcatggtttccttgtctttcacctgtcagtttgaaagctgctgtggagtgaggtcactgctcgcatttgagattttacaagaagcctctgacagctgtaatgtcattcgtaaaataagcgattagtttactccttctgtaaaagaaaataccaacacaaataatttaaaagacaaaaattagcaaacataaaaacatattaaacaattgaagtattaatagcgaagttaataatgttctcttctcttgttcttattttgtaaagaacatacggagttttagcttgcaggagactttatgtcacagggcgtttgtttggaaactgcacagaagctgatcctgaaggacccattgccgaggaccttgagggcttcctgcaggttttctcatgtcactgctctgacattgcttaggttacggccgtgcagctgcgtgccaagataaaactatgtatgggaataaaagttgtactagaaaattagaaataaagaacatggctaacgatttgaaacagtcttaatcagaatgtccaggctttgttttataaaaacagtgttgagtgtggcatgactcattgcctctgactcctggaaacctcagagttaggagaccttctctttccacatttggttactttaaatggattttcattatctttgttagtttagatattttaagtgtttatgagagaactcttctggagtaccatatagtttgctcttattttatgccagatgtcttggagagcatcatatcccttccagaggtgctgactcggctgctatttgtggggagctattgttttgaacaaaatcgaaagcatgacctatgaaaattgtgggaaattctttaaaactataattatagctatattttttaactttcatctctctctctctttaaaagaaaatgtgtttatcttatcaaagtatcataactatgagggtagtctaatttatcagtttttctaggctctcacctaatgtgtgtgaagattaaagtttttcgtaacagtaaataacaaataataaactagctgctaatcagtcactattgagtactttagcattaaggaacccatcagctagaatatttggtaaaaatcagttccttagggggccggagccatagctccattgttaatagtgcagactgctctcataatagtggccaaaagtttggtccccaggcgtcagatggtgcacagtctacctgtagttcatggcccactggcaccctctactggacttcataagaacccacattcacattcagagactccctgtacacacacacacacacacacacacacacacacacacacacacacacctctacctgcaaaagtaatcttaaaaaataaactccataggtaattcttaatataaagaagatttggggctttaacttataattttaagaagctctgattatcctttaggagaacagaaatgatgtacgagttgctttattttcccttgaaactggaatgcttttaattatggtagtaccatcagtaacattctaaaattccagtttcattatttttctacattttggtcacatctaaaataagaatgcagaattgatttttctttttagcaagagaaatattggtgaatcttattgttagtatattagaagaaaaccattcttacagctaatattagaatgtataaatctcacttattcactcattccagctttggaacactcaagtatctagactgttcctgtagaatttctcttatgtgttactcttttgataggcagttgttggtttagatcagattcttaaacatctggtgtgctcattcagcgtggaagactaagtggggtagggcttgggcgcaggcagtgagtgtgggaaaacactcacaggtccttctctgttgttcattgtgtttatgttttgagacagtcttactggacagctcaggctggccttgaactagaaattcttctgctgtaccctttctagtagcaggctttggttgtgtgtatcaccatgtttgactttaaagagcccagtaagtagcctaggaactgtgttactgggtgactactatcatgagccatttcggtccttattgacttaagtgtgagctgaatgctgtagaccagggactcaaacaggtgagtctcccagtttttgtggtgatcctgagcagtgatcttacctcctggcattccactatctctgtggtctatctacatacaccttttcctaacatgcttggcacttttcagctgctgtgataattaggtttggaagataagtctgggagcatattacagtccatttgtaatatgcttaaaatgtcttgtataatcaccttcaagatttttaacttaagctacacattgagacagttttactcattgaaattttgaaatgtttatgtcagtggttggattgttctctgttgccttgttagccagggatcacacctagtcagctgagttcacctggcagccattgagacaggcagggctccctcacgttggcgcactgtccttcctgtccacggcctcagtcagtgctttggacaaggacaaagtgacacagcacctcttcagcaacattgctttggtttaggatttgttcttaatcactgtgccattaatagagacaaagctgtattaaaatttttagtgttttttcaaatataacctaatgggctgttggttttgtatgtatagacttcagttagtttaggttgtatataattttaatcttgttcttttagtggactttagaccagccagttaatttttcagaaaaggcctgccataattattaagaaaatgtattgggtttttgtttgtttatttttttgttttgttttgtttttaggaagaatgtctaatgatcccgtttatagtaaaattttcatatgtatagagattttttttgtgtgtgttactaaaaactgtaccacaatcgtgaaggcctttcttggtaaagcaaaagtatgaattctgaatttagaaatgtgatatctccccaaattatctggattgagtagcttatgtagtaaattttattttcattgaacaacttacatctcaaaaataggacatgcttattgtatgcaaaataaaatactttctttataacttcctaaattattagtaattttactgctcagaaacaaccaaaacaataattttaaatatttaattatttttacgcaaaattggcagttagacccattcttactgtattgcttgtgtttctccaagactgtgagtactggtaagtgtctgatctgccacgtgaatgtggcagtattcttcttctttgctaatttgaatagtgacatgctttggccatgtgtatacaaaaaatttttagtcatattattatgcacagatttgtagatgtaggattacttggtaaagaattaactttattctgttcttggtaggtagtaatgaattgttttaccgaaaattgtaccaattttaaaataccacagggcccatctaaccttactgtaaaacatatatgtctgtttattcctatgtaaacattttagggacattacacttaggtgatgtcagttccaatatttccattaagtacatgtcatcatactccatgaagaaagcataagaagaaatctaaaattatttgttcatagtgatgatctttgttgtggaaaaggacctttgccagagacttgtcacctgtattatttccaactgtaaggattttctactcttttcatagctatgtttctgtgtctttgtccaaatagattgtccacatgtgtgattttaaaaatgtatttctgattcaacattgtctgtttattgctggtcttctgatagatgtcttggtttttatgtcatattatactcttacactcctggattttagtgagatatttttatagtattaatggtggcctctcccaaagtgtactgctattaataatctccactacgagagtccttctcctgcgagctgcagcagctgccggagacacttgcctgcttcttacccagtgttacttaggtcacatctgccatagctttgcccattagccactttctaggcttacccttcctccctctctctgctgagttccatttccaccatgattaacttgactatcttagtttcagcagttttcttgtttaaggtggccaactgcaattcaacttggtttctacacagttcagtgtctgtttccagaatgttcagctgaatatataaacacacacgagtatctcttcagcagagataaatataaaacattttaacacacatcatcccccctgtgttacgcatggatttttgtccccaggttctttcaagacctcacggtggagcctggctggctggctgcaccttcttccaggcgctgtgcttctttgtgccctagtggtcctcgtccattatccttaactccagtgcactcgctgttctaccattgcccgtggcctggtttgggctgctcagtggtgctgtgtgacactttgtagaaacacttggtggcttcagttgggctatccactcagaccacaggggctgactccaatactttttatgctgcaggggatacgcaggagaaaggtagtgtacagatttgtaggactggttttgttgaaaaagctgttaaagttggagtctcttctaggagttttcgtactcttaggagcatggcatgtttttgtttactgtgttttcaaatgactatgcagctgtgattttgaatatttcattttctaaatttaaccaaactcagatatagtttgacacttaattttaaaagtaattactacttaccaattttgaaagtaaaggctaaaacaaaaactcagtgtagtcctttaggaggtacttttaagagtcatttaggaggcatgagggcttgaaaatgccagaaaaggctaaaagagacaaagacctaatactcggaagacgaaggtcagtattgtggtaggagctgatctagaaacagcattgagtgaggatatagaaacattgtcttttgcaagttgcttaccacgtaatatgcaatttaaagtcctaccagtctttttcttcctgagtactttgtacagacatgagtgaagagagtagaaatcaggagtttcctggggaaactacagtctctaacatgcaaggcactggctaaaaatagaagctcctatacaggtctatacaaaaatttaaagcaaacaagcaatcaaacaaaaccctacaaccttttaagcatcttccgtctgtggtaaaacttaaactcattgtaatacaaaaggggaaatgcctaagaggcccactgtacaaagaggccaacagttaagaaagggcttcaggacgaagacatcccgagttttgcaggcaaatagatggaactaggaaatatcatcctgagtgaagtaactcagacccaaaaaggcatgcatggtatgtactcatcagtgatattagccaaaaaaagtgcagaatatccaggatacagtccacagaacacaaaaaggttaacaagccaaagggcccaagtgaggacgcctcaatcccacttgagagagagaagaaagcaatcatgggggtgggggggaaagggagggacatgggtgaaaatggggacagggagaggaagaagggaacatgaccaagtattgggtgggggaaaaggattgaagccctgagaaccaacagaaagaatggaaacaggcaaccttgggtggtaggagatgggggtgggggtgggggactctccagaatataccagggacctgggaggtgaagagactctcaggactcaaaggggagggaccttagatgaaataccctagagtggggagagggaacttatagagcccacctccagcagaaagatagacatcaagtgagggatggagttaccatccaactgtttaaaactctgacccataattgttcctgtctgaaagaactgcagggacaaaaatggagaagaccctgaggaaaaggtccagtggcaggcccaaagtggggtccagctcaaggggaggtcccaaggcctgatactattactgaggctatggagtgctcacaaaaaggggcctatcatgaccacattctaaaagactcaacaagtagctgaaagagtgagatgtagatatttacacccaaccaatggacagaagctgctggcccccgtggttgaattagggaaaaggtggaagaagttgaggaggagggtgaccttataggaagaccagcagtctcaattaacctggacccctgagatctctcagacactggaccaccaaccaggcagcatacaccagctgatatgaggcccccaacacatatacagcagaggactgccgggtctggactcagtcagagaagatgcacctaacccttaagagactagaggccccagggagttggggaggcctggtggagtggtggtggttggggacattctgtggagatggggggaggaggtactggatgtggaacagtcggagggtggaccagaagaggggtgggagagggagagagagggagagggaggattaaataaaagtaaaaacaaaaagaacttgagatatctttgtttttgaccagctgatctgagaatggcaaggcatgtggactgttgcttgtgtttacatcttgttactttctgttagttgtcctgctgccgatatccacctacattgtgccaataaacacaatacccaaaaccaaactgagagtttttctctaacctttggattaaacaggactagagagaattctgtgttcttctggagcccacagattaaatactcctacagatgctcctgttttgatgacagtaaagtgaggcagggagaacatctaatttggtataaggataagcagatgggatactactttataagtatttctcaaaaataccctcgtatagtacgtaagatgacctcatgaggtatactgagttatagtattcctactcacttgtatgagagatctgaggcctaggagggtttaagaacttgtcttaaaactagaagataagtaattacttttgaatgaggtcgtcaggccgtgggactagtatgttagtaacctaatcacagtagagtggattttcattgcactgctgctagtatcactttgatcttgcctttggtgtgatgccactaatggccactcaaagaccctgagtccatggaacagagttgagagccttgaatagacctcttcataaaacctgtgtccttaacgttcttcacataggatgatctcggtatgtaattatgacattgctttctggacctttttgtcatcagatcttagtagtattgggttttgcttcctttgttaatctagacataaagtaaattaaatgagcacgtgctatacaaatcacactgaggaaacactgagtcatgtcttattacccacaataagctggacagatgataagtagccttaataccaacaggcagggtactcacacctgcaacttgcattttaagaaatgagttgaatgtcaagactggtagagtgaacaacaaataaaaaaatactctaaaaataaacttggaagtaagttcctggcagagtaaattgtttgaaataagtctgcacggccccttgcctgcacaccagaaatctaacaagtcctagaaactaagacttaaaagacttaaagttgacgtatgatggcctcctttaatacttatctaagtgattattttgctgcagaaataatgtaatgtgctagaagtttttgtaatatgtagtttatgtgccgtattatctttctgaagtgcacatcaaaatgcacataagtgcattatctttcaaaatgcgcatgcatgaatgcagctgtcttcagagttagagaaccaagcctgttgaactgtaacattcataactatcatttatatgtaaaacttgaagactcttggagatttcaggacggtttgaactctctcttaagtctctgtgtatgtatgaatttaaagcatacattgcatggataaacacctaaatctattacctagttacagataggtcaatgtagtataatgacagtttgcagttaaaatattttcatcagtgattaaaatatggcagatatatacattaaatgatgggaaggaaccccagccacttttgacggtctgcacattaaaataagagcaaagctaaattaagtttaaccttccattacagattatgtctgttatctgcattttgctagtattgtagatggataagacagagtggcctcccataaggaactactgatggcctttctgtctcttgagagatgtatctcctgttatcgttctatagtcatatgtcaaaaacaccagaaggacaagctggatactatgtactgatgcaacacttggaattaagattggaagatatatgatctcaactcaacttgggctgcctagtgggaccctgtctcaaaaacaaaaccataggtacatgatttcattcattcatcagataggtattaaataagcaagataaccattatagttacaggatgcatttgtatttgggttgtgccaaaatacaatgtgtcagcagacactttgtgtgtgtgtgtgtgtgtgtgtgtgtgtgtgtgtgtgtgtgtgtgtgtgtacatgtatacagggctccacagaggccagagatattggatcacctagaagtggagatccaagttgttgtaagccacctaatgagtgcttagagccaaactcaggtcctccacaaacccagcacacactcttagctgcagagccatctcccagccctcttttattatacacttgtattttatgttaaagtttcttcacaagtatatacacatacatggacagacacactttgaattgttgtagcccttagtaaaatatataatctgttctcagaaattctagtacagaaaatacagtttgaattctattttaaaaaaatatacatgattttcctctactcatgtttctacagcaagactttttttgttagagagtctttagtgtcctgtcagtcacatcccagtggtttgcctctgagtcatctctgggtcagaatcaagtcctctgctctccctctccctgactgtgcgaaagttgcctaacttactgtccccagcacactggccagttaaataataatcaagtaatccaggagtgttgaaatgtaggatacaattttctactgagataatagcaatcacctatctgagataaatggatataagccaaaagaaacccaagacagaaaaggttaaatactgatcctaaaaggacatggacagttagttttgggttgtgatatctttatatgatggtactcagttctgttttgagaagaaaaaaaatatagaaatgttgaataacactgttaccttaaatttcttccttacaggtcttgagtgtggaaattgaaatttgttattcctgtgcttatttacgtttttgtgtgtgggtggttcataataatagtttatttgtgagcaatttaagcccaaacagcttttcaatcatccacagaaatctttaccagtttagatgtaaaatgtcattgttactactacagtgaacatataggggcagtattgcaccgtttttgtaaatcttgtctgaatttgaggatttttaaacagttgcagttgcagccttgttcttggacaaatgataacaatctatctgacttttaaatgcatggttgcacctgctttgtgaagtccaattcaggttcttatgtggattatgcaaaactcttgttttacagagtccttaatttgtatttgaagtcaaggaaatgttaatttaaagggtaaaattcatctgtagttagacaacaaaatgtaactaattcatgaaaaattgcaaactattaaaatcagtacagtttgtttttaactgctttataagaacttttcaagaatgtgagaggtttttttgtcaacttgtcaagcaaacaaaacctagaataatatatagtcttactttaagattcttcaattgataaatactatttttcaatatttaagtagtgagattattacaaactttgttatagatttttgcaggttttttttttttctgacttcagtgagagagaactgagcagttgtttcccatgtatttgttgttctcgatgctttcttcctgtgatctgaggactacttgacacactgagaatcaacagtgaacaaagcaaacccaggcttcactgttggctcatgtcagaagagcagagagattaagtatgtggatggtggtgctgtccaaacctgggcgtgagctcttactctttcctttactaagtgagccttagtaaatcagctgagtaaatggagagattactactatctcatagggttgctgtggtggcaacgaaagagtccataggaagtgtgtgtggctctaacaagagctcacgtcctcaagcccatgtggctaaagagcgagatgagttctcaataccagtatatcttacttttacttctgatatagtaataattcttactatttttagaattattgttttgaatattttttaaattgtgagagaaaatgcatagcatccattttgaatagtcagttattttttagtttcctgaatgactgataatccctgcctatagaaagttaatgtggcttggttcgactcacattatgtagattcattcagtgtgactaccctgtgtaaagattaaccccattatgtctatatcgatgagaaaatgataggtgacatctctgtattttgtttgcagattcccacatgtttagaataagtgtgttggcttagaatcccattaagtagaatttgtacaagtactaaatgagaattgaaaagagtttattggttcatgtgttctgtcgtttgcttcactgtctctagtcaatcccttccatttatgattttaaattctgaaaccctttctcttttttcagccccttaactttggtaattaagaagtggaacacagttgatgacatctttgaaaatgttctgatcaaagcggtcctcattactgtgtagagagagacactaaagcatttgaagaactcagctttctcatctgttagcaaatatacttaaacatttaggccgttctttgctataccttgctttcttcattcttttacattttagacgtataggaaccatgctttaacatgcttataggtatgaagccctgcacacacagggcgaagcattgttcaggtctgcaccctgtttaatagcttatcagctatgttcctgctgtgcctgctccccggaggggaagggttgttctgcaagctttcatattagctttgttaccatgcagtatttctcagctgctcacgctcattaccatacagctggtgtggagacgaggaggaagcaggcagaagttgtactggctacaagaataagaagctcttttgcagatggggtgctggcagatggggcatgtgtttgagcaggttttactaatgacagtgcgcgttaaatatcattctgaggaagtccaaggtcattgtcatgaatttagagctttcgaggttgtgcagctttgtgcagaacagaccttttcagagccctaattaataaaaatgtaaccagtggcaagcaacaactagaaacatctaatgagtttttctttattttagtatattttatctttctctgacatgatctaagaagcattaatcttatcaattagtctctcttattactttatgatggcataattttaaagttattaaaaatttcaagctatatacaaaagtatatgctttgaaaaaatacagtagcactcagttttgctatataatgttaaactattagaagatcaaatggcagaagaattggaaaactgtgtctggtgttgagcattctgaagattaagttgaagtgtgtgacacatcattgtaaagttagacagaaatggtatggacaggggtgtgtcttttgttaaaatattttgtgcttaattttttatacccataaaaacatatgaggttgagttgaaataactgccaacacttgctttttaaaaagttttagccagatgagggagcacacaactgtagcatcaggacttgggaggctgagacagtttgctactactctgtgctgcttggtgagtaccagcctagccaagactgtgtaccacgactgaagaaaaataacaagaaaaaattcttcagtggcttctcactccttttgacccttttaattaactgtagttttttatatatcatattacaagatgattaaaaagaacagctaaagcaaaatacagctgtgcagtcgtatgtaaaatgtacagtatcccctttaattttaatgtaaatagaactcttttattcccccaccccccacccctcttgatccagcatccactcgctctgggccctgcttgctccgctccggctcaatctggtccatgggttttgggtttttttttggttgattttttgttgttgttgttgttttttttttttttttttttttttctcattacagatggctgtgagccaccatgtggatgctgggatttgaactcgtgatctccaggagagcagtcagtgctcttaacctctgagccacctcaccagcccacaaatagaactcttattttgttaatggagattaaataatataaacacacattacatataaaatatacatagctaatttcaggattcatggtcagagcatgtttgcttacaaactttaatgcttctattttgtgttttgtttttttttattttcttgattagtagaggcacaaaattctgttaatgcctgtaaataaacaaataaacaagcctgtatgagattttcaggaaaggttatactgttaaatactttaacagttatactttttttcctttaaacactgttagagtatgttctgtttattgtgaaggtttgttttcagttggctttttaaagttccagggagttttcagccattccattgcatctcatcacataccgagatgtatttctgagacacataactattctctagtgagaaaatacataaaagataaaattataaataacatagaatacatataaatagccaacactttaaaattatttctgtaggtctgtatttatttggtagagtgtatatatatatatatatatatatagagagagagagagagggagagagagagagagagagagagagagtatatatactctacaaaatatatataatttatatatatatatatatatatataatatatatatatatttgtagagtatatcttacattgccaatagtctcctgtcaatattttttgttgacatgctatttgtacaacaagtttgggaagaactaagataacacaagcatcaactgtagtcactgctgcccatgtaacttaacattctcattgggaggacagacaaacataatgtgttacagtgtacctaggttccagcttgactgagtgacagctttggaatgggagacataaccagtatgcagatgacttaaaccatctcttcttcaacccagaccttctgagcttcagagccagttggtacatggtacatctgtctttggatatcttggaatcctgtcatgttcctcctcctgtcttctagaaagcagaaaacaatcaaacagcagcaacaaaacaatccttgttatgtcgtttatcttctgtctggtcaaacaaataagaaatttgatgccatctttgatactgccctctcctttgtttctatcatctgtcaccagtatgcccagtcagttttgtgtccaaacacctcaattgttgatccctgtgtatctcttgctacccagctagtccaagctgccattgcttcttgcttgtccagtgacaaatccttcttggagcctgcccatatcctcttccagtcactttacctgctttcaaccttgcatttaaaatcattttttaggaaagtttgtctgattacatcttgctttacagaagtccaatattcattggattttatgttattaaacataacttttcattgcttttatgttaaaataaaaattatttacaaagttttccataatctagtccttccttatttttctacccttattctgtgccactagtcatgcaggctgttccctgagcagtctctgtacccccggactagatggaaccttaacatacacattcttcaaacactacagcctactgcatacagcatccccctccctttctctatctcctctcctttctttttgctttgttgtctttctcaccctcctacagtataatctcaagccatgaaagccagtatttaattagctaatacagagtaggtaataaaattaatgcatactgaatgaatgagaatgtttgttggaaattaaaatagccatataagtctattggtaaaaattatacagcctagcaggatggaggtagagacagagaggataatgaagtattggtttaacttacagaggcgcatatttactaacaatgatatttgctattattagaatgggacaaatgtgcctgggtcagattgacatctgttaaatgtggtttggctcctagaaaaaggatttatgtgataaaatcaagaaacagggccagatccaggtgtgatggtgtctagtactggagagagagcagggcagaggtagaaggatcctgagttccaagccagcctggactgcatagcatggtccaggtcaacttgggctgcatagtgaagccttggaggtgggtagttgcagaggagggaagcaaacacaaagaggtagccatattatatactgtgcttgtacttaaatgtcctatgtgagtccatagaggaaatcctgtgactctggatatgctacttatattgttagattgtatctgaaactgaaaatatttagtatcagatgaagctacaatctccaagagtatttggattggtggagtcaatgaatagcacttaaagctgttttctttggttcagtagtagggtttgtcacttccatcagttgctcttgggatccagatccttgtagtaaggatgataaaagccttgcagcttccttgaatgggtgtttcattagagatcttcagtgtgtcctggtccctactatccttcagtatttccatagaaaataaagtctaggaccatgaaggaatgagggtagaacctaagttcttcaaagggctggagaaccagctcagcaggtaagagcacctgctgctcttgcagagggcctgggttcagtgtttagcaacctcgtctggtggctcacaaccacctgtaactgcagactaggggctttgacaccttcttgtggcatccacagatgcttgcgtactcatgatgcacattggaaatgagcaggggcacacacacacacacacacacacacacacacacacacacacggaaaatagataacattttgaaaaatttaaagttcttagagggattagaaaaaggaagcaaacttaatgtgttatgatcaattttatattttcttcctctgcccactagtaactgctgagtcttaatatatgtaataccaaggataaaggaagttgttcttagcaaggggaaattagcagttgtgtatcaactcttgcttcttttggtaaattcatacacgcagttcatcagtgaacacatctggctccctggtagctagtttgtcttttctctgtgtctgccttggctttggtgcagattccatccacatatgatacctttgcatgctgacacagctcagaaaacatcctagaatatccgaaataccaatatgtcttacctcaatttagagaactctattttattctataattggaggccaagatctggttctactaccatgtttttctggccctgggaaaattagtgaactcttggcctctttcctcatataaaatagaggttatcatcatagcactgtcttcataaggctgttgtacagttttcatgacccaatgaacaaaagcagctggaagaggcatgacttggtgagttctcttcccagtgtataccattttcctaaaagggcatctttgggtaaatgaattgcttattcagactcaaaaagaataacatacatatatagcctctttcattgtggctactgaagggacactcaatcatattaaaatctagtgactattttccctttattgttttagagtaaaatattatcacatgcctaactgcattatcatcatatgagctgaggaatattagttattaattatgcccatttgtcttctctgattgaccagcctactcttctttgagaatgtgggaggtcttctaggatagagacagctccatcttgatcagcttgcatcttgttttgcttaatgtctagatagctagaattttgctgactttggctcaatactttctcaaatgtggtagatgtttaaaaaccaaaaaacccttaattcctaattaattttctaccttttccttcatcgtttagaacataagaacccattgtatgactgagaatgttaggctgaagttgatcctattgcagtagagtttacggatgaaggatcactctgtaaatgattcctagttatcaccgtttcctgtgctacctgtgtgtaaacagtttagtagatccataaaatacgagttttcaaatgagtagagtagtaaggtaaattataacttggcctggcaaaatctgtttgttgcaattgtttaataaaaactaggagagtgaaaatgttttagaaagaaccatttaatttcatctttgctgatggtgatcatgttcattatcaagtcatcatctattttgaaatatctttaagattttttaatgtgctattttctctctataaaataatttttaaaggaaaggcttaccatgaagaagaaattatttatctgtgagtgtatgtctttatcttaagacctgacagatacctaaataaattattttaagattatattgaagttaattatctttttgatagttttaagtttgaattttagctacatctaatatgtgattattcttcttgtaaaattcacacgtagtgacttgcaaacttaaatttgtgaaaatttagaatttagttttaaaatgttctagcgtccttcttgtccagatccagagcaggtctcagctcctccacagcatctgcccctagtctgcccttccgttctacgccttcgtgtggaaaagtcaataccagtgcagaaaaataatcctttgaagttaggtatctaaacaacatttaaagttaatttttagacatttggggtccccaaggtcttactgttagatatctttagagcatgtcttatttgttttaaagtgaataaacaaaaggactataacaaaagattatgtgtaactctactaatggcatgtctgtttccagcaatatgcttttgtattcaaattgaggagattctaaaatctcctgaataagcaggggatcttgtaaacattatgctgctcataattcttgcaatttgttaaattatacacactcagtaactgctttaaatgtaaagtggtgcttttgatatgactatttagcacctttctttttaaacataatggggtgatcactgggttcttccgtgctcacttggacattacattagatgcttgaatacttaaaagagcaagtaagaactctgatgtacattcataaagagtgtggtgttttgtttattagcgtacaaccaatgcagagtaggtattcaagcgaagtaagtttatattcccagtcattataatgattagagtttcaaaaatatttttaacaggtcagttagggctgtgtagtgtgactctgtctcaaaaaaaaaaaaaggaaattgaataaaattatttgttattttaaattttatataattactgaattggtgaaaaagaaatgccagtttctaaaagtttagtgtttgaatctgactttatgtatctaccatccaaatattgtcacctgaatacagttgtcaaagtcagttgagcaacacatagcaagtcagagagttctagggctgatctgatgactccatgtcaaccaggccagagcaagtgcacttttgttgaaggtctcctttaacttggaggctctgcagttatgctctgtgttcttttatggtcacagagaatagttcacgatgaaatgtcctttcagctccaataactgaaactaagtgcatcttttattgtagtagacatttgctaaaacaaatactggcaataccataatagtcattggtcatctgagcccagtcgctcttttaaaaagtaaattatcaaatgcagaagtcagatgagaatgtagaaaacagaagagcagtgagataagggctgtaatggtggaccggaagtgagttagcagaacctcaaatggtgctttacaagtggtgcacagcatttagggattgggggttacctgctcttcagttctcttcttggtccaagtttagtggtctttgaaatagggggttgattgagagctggtttcagggtgtggtaaatcatgacaggatgaaggggtaaataatatatatgttgaaaacaaggcaaatactgtttatactttttaccatgtctcttcatgtgggtatgtgcacatgtgcctgcagatgaatagaggccagaagtcaacattgggcatagtgccttatagctttccatcttaaattttgagactcagtctctcacttagtctgatgctgatttggctaggcactctgggcagcaagccctggggtcttcctctcttcaccctcttcccccaacactagagttacagataggcacagtctactttgcctgactcctatgtaggtgttggtgatccaatctcaggccaatgcttgcatagcagagacctaattgtatctcttatttcaaaaagtaatgattgctttgtgaatgatcttcaactttacaatgctgagaatacacggaagaatatgggcagacagtatacagggaaattgtgttaatcattccattttagaagccttttaatgattcacaactttcaaactcttgatagtggtctgcaagatccttagtgttctgatcttgaccttttaccattgcttcagcctagacaaacttgtcctatccttgctgaacctgaacctgggctgaaaagttgtctcctaggtgctaccattccagattcctctatccttgtaactgctcacttgtttcatagcaccccgctctgtgtcttagcgctgataggagcttcctgtgtttgtttcctagtacactgctcaggcatacacaaatgagtgctcaaaatgcatcaagcttgtttatttattgacaggtgaggaaagaaggggaaagcttacagagcaatttagactgccaccattaacatttttatatttaggtttttcttctctcttcctaaaggggccattattgaacttcaaagtagtcctcctccagtttgatatgtaatgaacagtagtgtggtacgtgttttagaaagaaaggtttggaggtgtaaacgggagagaggcatggggttaagggctgatggtagaacaggatgaaaaaattcaaaacaaactgagtttatgaatagtaagggtcctgttagtggaaatcaattagtcatgttccccctctgcttccctgagatacattgaggagtataacattttaataatggttgggaaatttctctggactgttcccccactactgtgttcaagagcacaacttgcttgtatgtatcagttctggtatgagtgtatgttgtaacaggaagacctcttactagtcattactaaagtgaaacaaacaaacaaaaaaaccaaagcagtcctcatgcctacttatacattcctgtaagttctccttgtatagaaaggcttgtgggattctcccatgaattaagatagattggctaatacattaaagaacactgtaacttccacattaggtaattgagtcagtcgatggcagtacatggattaccatgtaatgaaccctctaagagcagtgatgagaagtcttaccattattagttctgcaggttagtaatggctgtaagtttacattttggttcacagaggggacctttttcctccccagcataattttgtcttagttggtggggttctaacctagctttctctacccagtgtctgccatcctttcctcattgccacttgcctcagacttcctacctgcagtagaggcatgcttgtaatgaccagggaaccagtcctctgaaagcagagcaccttttttttttttttaaagcaatgtctgtcagcttgacccactaccttgtaatgggaactagaaagcgaagttgagctctggaaacagagcccacatcctggcaggacacctgttcttactattgtcaccattaatggcagttaccatttgtagggtacttaatacctgttagacattcggagagtcctcacagtcccttagggtatgtaagtatctgaggagtactaccactcattccccacacaactcatgtgccaagtggccgaaggcacactgacgtttgagccaaagttactcctcagagtctgcgcctcaagttcatcacaaagaaagattagattagatggctccaggtccactccatggtatatggatggcttcctggtactcaagtgttttgttggttcaataggtaaaacactccttttatttcttagtttttagcttttttagatgcctctttgtcatgactaatcactaatttaagaaattagttccaggattttaaacatttgtaagtgaatacaagattggtattcacacacaagatttcatatacatgaaaacttttctcaagtagatttgaacttcatagccagctccaataaatatagacgtggatgaagaaaatagtaaattgggctgtaagctttccactgaaggaaagtaaatttcatcttgactgtgtattagttactattttgtttttcctaaagtcaaccttttatatttttatgacattttctttacagaaaatataagaataagtttacagtacagttttaagaggaaacaactaataacacatacatttaaggtagtggtattgtattttagatctaaagtgtgagatagagaaacgcaatacttgttagtagattgagggtagcttgcatggattgttggcctagtgcacaggtgtgtactatattgtgaggtatgctgacttcctaccaaatccctgacaattctacagaaatatatatcctagaagcaaataaaacttctagctttattttcttctgtgactgtgttaaatgaaactctaaccttcattattgtgtgtaagttctgaatgtgtggacatttatatttttgtacattcacatacatgtgcacatgtgcatattataatctttgcattgactttgtaattgattgtgccagattaccagaggctgcatctctctctgtaaagaggcttttagatagtttgaacttacagaaaaataaataaatactatttttacagttatatttagttaaataaaacttttaaagtctgagctttatgttcatttacagtttgatttactatggttacaatttttgtataaaatctttttgtgtttttcatatttattttaaattatatattgtgtttaatgttgggtagctatgcattaatactatagtgttggtttagttggagggtattttggaaaacgagagttcgatatttttaaaactaattttattatgggttctaaaagctccgcttccccaccctgtgtgtggggtgtgtgtgtgtgtgtgtgtgtgtgtgtccatgcccttgtcctttcttattttactatacatttaatagacgaagaaaaagtcttaaattatatactatcatgtgcttgacacattttaagagctggctaatttcctaatgaatgtaggaaatgccctactagccagtgccttgcctactgcagtagtgcatgcatgggtgctgagctgcacacctccttcccagctgatagcacctgcaagagaacactcccactgactttccactttagtcaggagcctttccataggaaagttaatgacaacgagttagtaatgattcaaaaagtttgttacagcctcacatgcttggcattcttttctttgatgtagaaaatttgagctgtctctaatctaattctgctcagggcaagacagctaactgtggtaggtagtttcctgttcttttccagagtagaatgttgtcatgataataagatcactgacatcaaaatacaaacacatcttcttactattataaggtagaaatgtatttttaactgttagcaaggtaactaagaaaataatattcatggtacaaccaatcctattacagtaaacttggaaatactaagtatagtagatattaagaaacatatactttatatattcatacatatgcctgaattaccaaattactcttatggttgtactgcttgcttgaaaggccaattgtttaaaactgttatttgttacataaatgtttttctggatgtcctacagataacttgagtgatgtatgaaccttccgggagtaattattagagagaaagttgtacagagataacattaggttgttgtggcagaagactaccaagactagttagtaatgacttggaacagtaattgtgtgctgtagggaagccaggagtctgaaggtcatgccaaggaggatagatgcaattcgctgtatgtgcatacttcacttgcatcatccacagtagactgttttggtttgtttatgtatataaccaactaggtgtccagccgtttgctcagagcgagtcatttgcttagactagcggtggttaaccattctatgccattatctcccagagcttgggctctggttcaaaacatctttgtgctgcttatttctggagccagcatccacttgtatacaaccaactctaaagcttgctaataggttttaatttaaataatgcttatgcattgctagtacaaggaatatttcaaaagaatgggaaaattattgcctacctggaattgcatgaaaatttattttattataatgaaaacatatttactatgttgagatattctaaaattttgtacaactgacattagcatttatttatctatagtagaaactcaccagctaatctttttgatgaacttctacttgatgttacagcataagacataaaatacaaagtaagcttagaactacatagattataggctaaagtcccatgtttctgtccatccaggtgtaatatagatagaaaaaagtatttaaaatatattactaaaacacctaaatatgatttatgcttgagaccatctgtaagctaagacagccagaaatcagacgatgtgaatgctttgctttgcctgggtcatatgtagagtctggtagtaaggagatacgacgcatggttttcagtgatgctacttaaaaaaagagtgctggaaaaatctttatttttattttgttactttgaaatctgattaggaaattggttaactagttttgcttggctagttctttacagttaaattagcattgtaaactaagtattgggagaattgtttaaatatttggtaaatggaagtcatcattagtaattataaccatacatatttttcacttaaagggctaagttaataggggccactcctaagtgatcaccttggcagatgaaggaagtgaaggaagtgggatggtatagactttccagtgtatccttttcatataactttatgagcaagtttccttgtaacttcatgaccataatcagcagttatgtgaccatgagaactcctttgaataaaggtatttctatctctggcaccccactaagcttacccaatacaaattctataggagagtccaactacttgttaatattcttcagtagcctggaacaagcaagaactccatatttgctgcccccaccccacaacccctgatcttgaagcaactagttaataagggtctgtatattttcccaaatattggacacaccctcgcccccaaagcaagaagtttcaatttaaaaagaacttccctttgcagagagtgaattgattgataggaaaaataacttttgttgagcatgtatgtacatattgtgtctgatggggggtgacataagaattaggtaaatgtttgagaagataatagacagtagaacataatttcttggagagaatctaagatgtgcatcttcataaaatttacatgtggaagtaaacagtattacattgaatgtcatgtttataatggtaaacattttaaaaattgatatttcatgtaatattccattaccatgcagaactactaaaatatattgttcaaatagtaggaatctattttctaaagaaactaagtgctctaagtaactatgggcacatagcttacttgtaataaagaacattagtggggaggataggagttaattaactgggcactagataatagctttcttatccaagaagtcttatcaccacttccaagggcagttgtgtagcaagttgcaagatgaatacctaaatttgtacataatcattctatgagaaaggttgcctttaggcaaagttagtttctttattattacataaaattcttttttaatgctaagcaatgtgcattgccacaatcatttttgaggtaaaagtctaccacctcttgttggcccagggtgagtgaatgtcaggggattcccaagtgagcacggagcacatggagaaagtagccttgatacaaacatgcattaggtagaagtggttcatctttaagggtaacccttcttgttcccgagaatcactggcagttatcttgaaaactatattttaaaataatgtgtggtaaagcttaagaaataattttactaaatcataatataagagacaatcaggtgaaatttatcttttattacataatttctgtaaaagaaccttttgctccaaactggtttactttgttgtcagttcccccaagaatacttaatttttccctttggaattgcccatatcacactgcccaagctacttgaagaatatagtcagcctctgccaagtctatccagtaggatgacagtaatgaaagtagcctgcttcgcctctgttgggggtgggggttacccaaatacatttcatttccaaaatgtttctttctggaaggacttatttgtggtaatgctgattgctatttaaatctcgtcaaaaatgtgggttttacaaaacacaaaaataatctcctgaaagtttggaaggggagaggggctgtggtgtgggggcggtctctgctgcccacgtccgccctctgttcagtgcgttgtgtgcttacgggctccctggagcagatcaccatataattgatgtgcagtctgcattgctgaaccctggactgcaccattctgtgttcaagggaagatgtaatctgatccctctgctgctgagggaggaatctgttcagtcaaggctttgacagggcatagtctcctccaataatcttgtgggttctcgctcattattccccgagttctcctcagtggagctgcatgcgtgtgtgcgtcccgagcagcgttctggttctgagctgcgtctgctggtgctggggagttttgctgccggttccgctgcctaatcttccttttctggcgtgcctgagcatgtccacgttagaatctgtgacatacctacctgaaaaggggttatattgtcagagactgccaagcagccggacacacgggggcacagaatccctgaaggggaaaaatacagaaaatatgggtttctacggcacattaaaaatgattttttacaaagtaagtaatctgttttcttcctgtgctgtgtggttgtgtgtgtgtgtgtgtgtgtgtgtgtgtgtgtgtgtgtgtgtgcctgagaaggaaaatctgtatcatcagagatggctgcagcatcctctaattttggtgggtggttggattctggtattctgctttatttcatgactgttgtaagcaagtatggagcgatggtgtgagctgggggaagctttaaaataaaatgtacatttccatctgagattttttcactgaaaacgtatctgagactccagaaatgtagctccgttaaatgctgacctggaaatactcagcctcacttggctttccgtgaacctttctgttcagttttcctgtaccatagtcatacgtatgtgttccttagttaactcttggagtgtagtcactgaaacgtgtagggtttatgtttgaagagattggactacttgtggtatcttaaaaaaattacccaattaatttttggttataaagtcagtattttcagttaacattacagtatttaaaagatttccacagtcattcagaagatgctttgttttgtaaaataatttaaacataaacccttctcagtgaattctgtgatatatgttttaaatattttattcattgaaagattctagatggaaataaagtttgcttttagtggcaattagatgtcatttgtaccagcatatttacaaggcgttctgggaagttctgagagccagcattactgacttaattcagatgagtgtcagagactcccataatgatcaggaaaatcatctgcgaaagttaccaagctctgtactggctgcccttcctgatggattcatttgtgtgtaggtacatgtctgagtatctgaattctacacgtggaagcacatggctccctgtgcattacagcttctatattttttgttttctgataagattatttacaaagagaaaaaataatatatttggagattatcttttaaaaatgaagtgagtatagaaacttccagagggaagaaagcaggccaattctgtttcccacagtaggtatttcactaaagtataaattgtgcataagcttcttgaaccttcgcagccacagatgacccagtgttgactgggacactttcttcacgataacaagtccttgctatcccatcccagagtttgaatctttttaatgccttttagtatttacagtgtatatactatgttccatgtgttcctgtgtgaagcaataggtaggaaaatgagcctactgcaaaataggggttccagcttactataaaaatgaagatagctatctccccataacatgcagtaatacagtagctagtgagaccctgcaaatccaagagaagctgttgaaataggaaaagttgaaccttaacagattttattttgtatacatatactcagatgacctacgttaattgatagtgaaatacaaaaataaatatttcaatattgacattgacaaatgcaattttgaatgtattgatagcacaacatggaatagtgagaaacctttaagatgtacatgttagacatgtctcatatatcatatgtcttagtcataactcattcttttacaatcatttcgttgtttcaagagtattttttcctgcgtattctgtatgtagcagttgatgatttttttccttcagctttactgtggcagatacagattgcctgctattagggatttactctttttaactgtctctgttatccttgctaaactcttctttattttaatcactattcaagattagcttattttgctctctacctttgtataatgatatgaatgcttaaaattcagttattcatatttctggttgtgaaatttaaaatttttatcttactatttttaaagaaaaatgattagtaaggaagacatactaaatacagttattttctatgtgtgctaataaattttttcttgttattaaactgaaaaatgaaaagtatataacattctaagggcatggtaaatattagtccctcacctaacactgctcttcccagtagaatactaagggaacattacctatccagtccataagagtaaagcaaataggaaaaactgcgtaatatattttctgattaacctactaataatcatcattttagacacatgaaaatttaacagtttttaaatattagcatgcaaactcaaagtctgataatggaccaagtaattttttttattttatctgttttactttatagatgaaaagaaagttggaccatggttctgaagttcgttccttttctttgggaaagaaaccatgcaaagtctcagattataccaggtaacgttgaatagaggggtaggcgggcatgtcatctggaaacatttctttgaatttgttttcgaagaataatagtgctactgtattcagtgttgttggttgttgaaatgtgttttaacattacagaaacggggaagggcatgaggctcaacccaagcagaagggctggcactgtccatcttggactttctcactgatactctgatgggagacacatgctggagttctttaaacaagattgcaaagtcacaattttaaggacttctaaacagtaaaccgcgaggaaatgtctctggttactatagcaactgctgttttagggtagctactttcatataccaaatcgaagataaatttattagcagtgaggtaaatctcccggttactaaaacttacttcatggaatgaatgtattagaaatccccatttattggcatgatgggttttatgttttaacacatacatttctacacttgtagaatagtttccataacaactaaatacaattgtagtcttgtttccttcttcactccatcatttttggattttgcgtcagtataaaactttgcccactctcacgctaattagactaacttaagcacaatatctcatttcccagccaattaaacatggttttggatgtcacttgaaatgctgaattttaacatttaaatgacaaaagtatctaagaactctgtggtcccctcccccacttttgtataactgtcacttttattgaataactggtcagagtgaacagtagaaattccaaggacattatggttaggatctttttttaaaaaaaaaaattttgatttagttatttttattctgtgaaacagaataaaattaacattgcctagatacaagattttctaaaacatcattacaaattgtcacacaccccatacatacagtttgtttttattttgttcaattgtggaaatatatttaatgctcctgggtaatcaaggaatcatactgcttaggataactttacatacagaaagctctggcaagatgatgctaaagagatgaagaaagcttcgttctctgtacagcccttctcccctcccctctgctgtctgacaatttcactgttgggcgcttgcttccaccagagggtgtggagggatagagagacaccgcaaatattcacgtcattttggttttccttctaaccttgatgatatatttctcagatgaggaagttgtttgatgttaaactaattgtaaatgtaatttttttaatgcacagatttcagtctgttcacattattttggtcttctattaaagacaataccaaaacaaaagtattaaatgatcagctgttttagaataaaacttttggtacagaacacattctcataaaatttttgttgcctactttttatatcaagacagcaacagccttttaacaaggaatcctactaaaggagaattagagtcaatacatatttatttccatgtctttgacacaattttgtcagttagtaacatatatgtttgatattcagtaatgattctgacaggtttttttttgacaggtaataagataaaactcattttgtattctcctgcctccttaggagactttaactattaacacagcctgttttattttggagaaaaagaaactgaatttgcttttggatttgaatgaaggaaagattgatttgctagttgaatattagagattgttgtaaataaatattaaaaaattcaaagggtgcaggcataaagatttttctataaaatagtgatttatgtaccgttttaaaaaattaagagtttcttcattagcgatttgtcagaggttgaaaatcagttgttatggtacaggttacatgtgctctttgtgtttcttttttaattcttttctttttggttttgttttgttggggtttttttgttggtttttttttgtttgttttgtaaacaaattcactgtaacccagcctgacttagaattcactatgtagcttgcctcaaacttaaaatggtctttctgcctcatcctcaagtgctgtgataacagatgtgagctgtctgctctttggatttaagatactttattatcatagttcagtgagctgaggattcttccacaggtctagttttgttgaacgttaataccacctgtaaggcagaaatgctcacattacctggtttgaggtgcttgaaccagcttagaggagccaccagcgtgtctgtgtgtgtgtgtgtctgtgtgtgtgtgtgtgtgtgtgtgtgtgtgtgtgtgtgtgtgtgtgtgtctgtgtgtgtgtgtctgtgtgtgtctgtgtgtgtgtgtgtgtgtgtgtctgtgtgtgtgtgtgtgtgtgtgtgtgtgtgtgtgtgtgtctgtgtgtgtgtctgctacagtctcagggctggagcagaagcactatactctgttctgagttcatttatttagtgtttttcattatctcagttaaattattggataagaatgtttagttatctcttttaattttgtttgaaattctctatactttttttgaaaaactatgtctcatcgaggctgactactaccagatacctaggctcagcttaataaatgacaaattaaaattagcaatggataaaatatcataaataatttcaatgcatctggggcaagcttaagaacttatgtataaccagtgaaatataaaatattttttttctgaaatattggtaacaatactttcgtacttatttttacaaagaaaatctaaacttaaactgagtgcttgaagacgagtcatggaagtaggagctgtatgttctaaagccatgtatttttgcctgcatacttagttatgtgctcacttacggttagggctggacctggcctgtgttctattactgattgcacctgcagccctaagcaaatacactagcaacttagagctttgtcatttggttgccagtagcattgctggatggtgataaggaagggactttagtgaggaagaggaagaggaaaaagaggaggaagaggaggagaaggaagaagaggaagacaaggaggaagaggaggaggaggaggaagagaaggaaggaggaggaagagggggacgaagaaggaggccaaatgtgatggcacatgcctgtatttctaacactgaagagtttgaagcaggaagcttatgagtttaaagctagcctgggtgacacaacaagaccatctgcagagcaagagaaaaagaagaatagcaaagcaggctttgggtgtccccgccctgagagtagcaagcgaaagggcttgctctactgatgtttgaagaacaccagatcattattctgagtaaattagagttcatagtttttaattaagacctttcttccactcttacacctaggctcagcttaataaataacaaattaaaattagcaatggataaaatattgtaaataattttaaatacatctggggcaagcttaaggtcttacgtataagcaggaaatccaaaatttttaactctgaaatattggtaacattaatactttcatactttttattttattttattttttagtaccactggccttgtaccatgttcagcaacaccaacaacttttggggacctgagagcagccaatgggcaagggcagcagcggcggaggattacatctgtccaaccacccacaggccttcaagagtggctgaaaatgtttcaggtgactacgtgaacacactgttgaaatgcttcgctcgtttgcttggggtcagggtgggagtacagaagaatttggccattaacaaaaaaagtagtgtgtctgatgctctaccatttatgagcattggaaacattggactaaatgaaagaagccagatgtaagcggaaattttattaataaaaagggacagaaagtaggttagtggttgccatgtaggaagcagagaggcctggggctacctgctaagagatgtagggtttcttttgggaagggtgacagaagtgttctagaactgggtagagtcgtggtcatccaaaaagacaggcatcctgggacccccctaggtgtacacttaaatagcacgcctgttacacggcatgcatcttctaaaaccgaagcacagtgcttattttgtgtaagtgtatatttgcatggctttattggttttctaaatagtacctttattttcttgtttgtgtttgggtttctatgttgtggaatgtattcttgtttttggagcacatttcctgtgtattaggaatgtgaggttagtttaggaactccatcagtgagtcgctgggattggtaacttagcattcactgcttctcggtagaggtgactcagtgctttctttcattgccttgcttgcagatgccttgagttcgctcaacttccaaccactgtcttttagtaggatttaaattctttgaacatttactgggaagattgccttgaatactgtggatttttttaatttaagttagttagattgtcttatctcttcttattctgaatgatagactaaatcaagaacataccaacacagctatcatagatgaattacttaacaattctttccagacagtaattaataaaaatatgaacatgcctgatgctagctagccaccacaccaatataaaatgaacacatttcactacctcttgtctgcttgaattgttgggtacataatgcacacataagaaaattacaaatttaaaagatacattggctttgtcatttcaaatatttaagaagtcccattgtataaatcctaatattttaaatacttaggtgctgtgtgtgtttgtttcaactatagctagaaaattttcttgaagtgatgtctttttaggtgagttttaacactactttatgggatggggcagatctagctactgtaactgacaggtgtggagccctggttctcagtagggtttgttttttgcctcgcaaataataaggaaggggttggagccttttgatgtttactgtgcaaaagcaatatgaaggatagcctccaaaagcaaataagtatccaacacaaaatattatagtactaaggttaagagtcttgggattaaacaaatggaagttacattttacttatgaaaatgaatactaccacaaaaaacctgtttttaatgtttatagacatgtgttaattaaactcacctttcctgtaagggttactacttccccactcttttacctcaaatttatgacattttccccctacctatgccctgcaaattctgaactttttattctgtccagtcatttagaaataaataaggcttgaaataaaaatctagacatgtttatatactcttaaaggaatatttaaaatactaatatctgaccaagccagggaaacgggaggaatgacaatgagttctgcatgcacatgaacatgtgcacaagccagcacccagaacacagccctggggtcttacctgggtgctgaaaccacatggtgtctctagtgcccatttccacagccctcaaaactgactggaaaagacactcattagcaggctattccaaataaggttttttatatatggttaggcccatttttcctacagcattttaatgtaccaaaatattttttgtaaaccatttatgatgttcattaagagaattctttctaatattgatctgcaatagtgggggtattaatgctttcttctcatgaggctgttttagagagccctttatacagtggttatttaataaaatttccaaatgttatacttgttacagctcagctttagaaatgttaactataaaagtaaaatcagaacacccaactgtgagtctctaaacccatttggtcctagaaaagtatgtcttttatatgagtagttgcagtgagagatcattcttttattttatttattcactttacatcctgattgctgcccccccccagtctctccccacctccctccccttttcttctgagaaaatggagtgccccctcccccccgaaaacccagcacatcatgtctctgcagggctaggtgcttcttcccccactgaggccaggcaagacagctgagttggggacatattccacagacaagcaacagctttagggtgtagtaggattgaggaccctgaaggggataggaactccacagggagaccaacagagttaactaacctggactctcagagactgaaccattaactaaagagcatacatgggctggacctacaccccccaccacacacatgtagcagaagtcagcttgatcttcatgtgagtcctgaataactggaaagagctcatttttagttcatagaaacaatgtagaattatctgtgcaagtagaaaacgtaatcctcctgacccaagacattctgtagcaactaaacgtcaacctgacaaggtagatccagccttgatcttctgagcttcaagtctgtctctgacataacccaggactcgttgccatacactgcccttgtcttagctagagttttattgctgtgaagagacatcgtgaccacgataaaggaaaacatttaattgggactggcttacagtttcagaggctcagtccattgtcatcatggcaggaagcatgacagcatgcaggcagacatggtgctggagaaggagctgagagttctacatcttgatctgcagtcagcagaaagaaactacatgccacactgggcatagcttgaatagataaaacctcaaagcccaacccacagtgacacacttcctccaacaggccacacctcataatagtgccactccctatagccaagcatccaaacacctgagtctatcagagccatttcccacattgttctactggctaaccagttttgtttcaatttgacataagctagtgtcatgggagaaaggagcctcagttgaggaaatgcttcaatgagatccagttttaaggcattttctctattagtgatcactggggaggacccaacctcttgtgggtggtgccatctctgagggtctgtaagaaagcaggctgagcaagccaggggaagcaagtcagtaagcagcattcctccatggcctctgtcagctcccaccttcaggattctgctctgtttaggtttctgtcctgccttccttcaataatgaacagcaatatggaggtatatgctaaataaacccttccctccccaacttgctctttggtcttgctggtttgtcacagcaatagaaaccttagccaagacaaccctggttttgttgttgttgttgttgtttttgtttcgttttgtttcgaggctggtgccaaactaactagaactgactcacttgcctggaggcttatgatgcgtcagcgccaatcttatagatagccctagctcccgaacaaaggagggtggtatgagctgacctatgaggggtacactggcaatctttgtacaaatctgcgattgaggcatttgagggtgacttccctgttgtgtaacaagcaaaatgggagctcttgtcctcttcctgccctcctattgctcctaaggcagtgttacagcacttggtccagcctcctctgtcctagtaaagtactctcaactccatttgcaaattgagtaagaacaaacgatacaccactgtagataacagcctgttaatgaaaaacagaaatgtcaactaaaaacaatggactagaaaaaatacttctgttttgatatttataatgaataaactatatttaaagatttaccatgttctatgctaagtcaaattacacatgttttctgacttgttttacttaatttaagtcttgttagtaacagaagattaaaagctataagagaggtgacaaaacaagatcagtatttttgtttctttgaaattttgttacagcatggttaaggaagcttattaaaccttgtgtaacgaagttgaagaactttgtgtatttagtcagggaagactagaggaaagttctgtatttaatatagtctttacatcaaaatagaaacagattgagccttgggttctttggaaaggaatacctagatcacgttttctgagttggattatgaagctgtttgaactaagaagaattgcccacaggagagggcaagaactgttcaaatagagatggggaaactgggtgtggtggcttaagcctgtgatctccagtgcaagatggtgtctcaaaacagcagtcaggtagactgactgccctgcttaggttctaaagaagtgagttttctatcatgggagttcaggctcagtgctgtccaaggtcttaaaaccttgtgcctgattggaacagtaagcacactctattagtgttccctcgtgctaattcatgaatcacctgcattgagagagagagagagagagagagagagaaatcagcagggcatgttgatacatgtctttaatccctgcactctaggggttgaggcaggcagatctctatgagttggaggccagccttgtctacaaagtgagttcatgacagccagagaagcactttcttgggggttgggggtggggcatctccttcgagtaaactgtgggatcctgccagttttggggaaatcactgtgccagaaaaagtctcttattatggaggcttaaggctgtaagtagaagagggactttgcctgggaaatagaataggagagtaaagataggaagcttgagagaccatttggagatctgaatatcataatctagaagaagtgctggtagcttagctggtacagtaatgactaagtcttagatatgttttgaacttcttgaatttgtctctgagcctcctgcatctgcattctgctttcaaatgcttaattaaaattgcagttttgttgagttagcatagttgttcatctcccaaaatggccttggctatggtattgtctacagatttcttataatgtaggataggtaaaataaccaacatactatttttttaaaaaattcaaattctgtgagggacgtgaatcaataaattgtaattttaagttatttatactttatggtcagtgatgaatcaaatttgaccatgttgtcagatgggcagaatctgtggcaatgcctgaatgctctgggatcagacaaccaatccagtgctttggctcctgggtggggcgttcttcgtgttgcatttcaaagctgtgttgtgtagagattgagcacagagccctgaggacaagtgaatgctttctacattgtatgtgcaagagaaaggagagcacatgacttactggagcccccccccctccttacactggcaaagtagagtaggtcacggacatagttcagtgctaacccgagttccatagttgttcacatcactgcagagtctaagggaatacatacctcttgaattactctgagtgccgcttcctgcaagcctttgtgtttcttacatcttatgggatccgagtgcaagttttagaggaagagccataccttgcctgttactctttacccatccatgtccaaaatggtgcattagcccctagaggtgtcttcatcagaggcctgtgttgatatgttttcacgtaacaattgtgaagttacttaaatgttttacatggttattataaaattccaacatcttaaattattcatatggccttaaatttgttcctttaaacagccatggcttttgtgagcagtcatgctcactgagggaaaatattaaaaacaaacaaatgggaagaattttttaaaggaacccatacttttgaagtatatgtgttttcatttttcataagtacagcatttttattcattttgggctttgattatattcaaattttgcagtattaactcagctaatattttctattgcttgccattttttaaagtaaagcatttttaaagtgtgtagaagatacttgtcagatatgcagaataaccaactattaatttttttatgttgcaaatctcttagctcttagatgaaaaccttattcaagccataaaattttatggctgtccactaaagagaatatatagagaaaaatgacctatcaaaattccctatttattcagtaacccacatggatgtttctaggacaagattttcgtgtctcccaatgtcttggtgttagaagaatgcataactctcataattaaaaataaatgcagactttggtttttttgtttttgtgttggtttgtttgtttgccagtcatgatttttaatgctttttgggaaagttactgagtaaacttgctgaaactaaaacctgcagtcgcttcagtggcttctgtccatggttccactgctcaaacatggccatgcaggggcttgcgggtcttttgtatgaatttgtaatgtgtccaattcctctttggtagagctggagcggaccagagaagttgctggctttagatgagctcattgacagctgtgaaccaacacaagtgaagcatatgatgcaagtgatagagccccagttccagcgagacttcatctccttgcttcctaaagaggtaaggggctgtcaaccgcatcccgctgggtgctgtccagtggttatgggtgcataaccacagcctgtaagcagtgtcctgctcattatggttgacacttggacaaactgctatagccttttggtacagttgtcacataaagtgcattcataccatggtactacttccttccacttggataaagtaaggctttgtgtgagagtcactgagtttcttcaagaattttttccctaggttatctaaaggctggctttagttatttggttatttttttagttatttagttgtgtttattatttatagtggatgttttcaaaattctgggttcattaagcagcaacattatttttgtttttattttgttaattatctaatgccccttcaaatcagaaattttataattgttgtatttctctggttattacattatacaatttttttaacatatagaattttacctatattcctttttggcatttctactaatcatcatccatcagagagagaaagagaggagagagagatgacataaaaattggattcttacagctcttaatcatatagccatgtgtggcttatagtttaaatataatgcacgtattgacattagacttaatagcttaatttttatcaaaagaaaggaataatgtgatattaaatagtatcctcatagcctatattttttaaaattactatatacatgtaataaatatcagttaaatagcacaaaaaactaacacatcattcatacaatgatcctgaaccctcaaatttgaacagaaagagttttgcttgctcttttcttagataattactctaaattcctagtggtgttaaatatagtcctagatataatctggcattagatttgagtaaaatcagtggagtgaagtacaactctggggaaaagcaaaattgatattcccccccccccccggtatcttttcccttttggagtactttttaaaaatgacattgtcccagaagaaaagtgtttattcttgtgccctttttctacagcagaactctttgaataaacttagaaaagtaacatgagatctcctttgcttccatcggggcgtaaaataataggggacattttttgtaagtgaagtagatattcatcttgattattttacatggccgataccctcctctagaatatgcatatgtgtagtatgttcatgtgttcaagtgggtgtacatatgtacatgcagatgtggtctaccactgaatcgagagcttacccattcagctagagtggcagctgtccagtgagcagtgactgtagctagaattgccatgcctagccttttacaagatgggatccaagcccttatcttcatcattacacatgaaatgttactgactgagtcatcaccaagctccagaatttgaaagtgttttagaatttaagaagtaagcacaggtgattaacaaaaggcaaagccccaaaagattctcttgccttcggtgttctttcaaagacttcctgaggaaaaaagatgttcggaaggagagagccctggcttagcatatcagctatggtgttttgtttcctgctactggggtgttcttcacttgggaagtgctttatgtctatgtggaagaataatggtataggcttaaccctatagggaattatgttatttaaaactttattaccatttattacacaaaatgtacttgtatatatatatatttttgataattggtgtgaacaatttcaagtctctttatcaacatttatttaggttatctaagtgcttgagcactaggctcacacaagaaacctgtgataagtggcattgctctcaccttgtaagtaaataccaataaaatcacaggtcacccagaataaagcctcagattcctaatatgagattagtgagcattttctttgctcttaaaatcctgcttttcttgatttgttcagccactgaaatgcagtagtcctagcaagatgctcagcctgggtagagtaacctttttttctgtgattttttttttttttttttgaagtgtgagtatgccttccctatttccttttccatagttggcactctatgtgctttcattcctggaacccaaagacctgctgcaagcggctcagacttgtcgatactggagaattttggctgaggataaccttctctggagagagaaatgtaaagaagagggtaagttcagaatcacacacagatgtgtttctactctggaaacaagttaacagaataacttggctgtgagtgagagtagatggctggctcaggtgcctgccagaatgcgtctgcgtctgttcacttccttgatcccaacaggaaaggaggccacctgatttaaaggaagccacaaatctgcagttaaatacccacagaattgagtattgtatatactcctcgaataaacacttactgtgcacctcctgtgagctgaacatttgtaaatgctggggacatggcactgcttcatcctagtctccctgttgtggaccttctattattaagtatagtttagtttggcttcaaagggagaatcttctgttttgtaaacaggtatctagaaaagtaatcatttagaaaaaaatagctgtttctgaggggcaaaatgcttgaggggggaatggtaatgctctgttctatagatcaggccccttggcagccgtgtgaccaggtagagagcactgacgagtgaggcggaggagggaagactccagggtaggatctcaggtgcttcttgctgagcctggttctacagtcaacctctggctgttcctagattaactgataagatcctcttcttaagttgtgcgggttttttgttttgttttatgtaaaccacggtgaatcaactctgccttaccttctttctcagaatcttttttccccttgagacagggtgtctctatacagaccaggctggccttgaactcatgaagatccactttcctctgcctcctgaattaaaaggatatggaattaaaagggtatgccacatacctagcttaagagttctttttaaatgtcctcttaaaaaactatatttcgtaaaaatgattctgaacagcttggtttatatagcaagttccagggcagtccagactgtgataataacctgaggtaatggggttcattgtactcatagcacttaggaggcagagacaggattgctgaaagctagaggccatcttggtctccatagtgagttcaagccattttattgagtaggatgcagtgatattctccccattcttccattcctgttgcctacatgctttataagtttactgttgtaggaaactatgataagtgaaattatacaacatttgtccttttgtgattgccttattcactgagcaaaatgtcacagcgtgtatccccgtgtcctctgttaaggcgagcagcatttcagggtgcactgctgcatttggtgttcatctatggaaaggcacggtgacttgtatggacagccgttcacatctctgcttttcatccttttgcatatacatgcagaagtgaaagtcctgcatcctgcactgcgtttgtgtatttaggagtcactgtgctaatttccacagcagtgcacatacagtctcatctttgcgtgttcataccagcacctcccacagtcactgtgagagagaatggagatagaacaaatggaggttcagtgctgcacagcccagtatctgggccaagcagagaaaacaagaactttataagatgtgtaatttttattagtggtgaaaagaattattctcttgaactctttgatactttagggattgatgaaccgttgcacatcaagagaagaaaaataataaaaccaggtttcatacacagcccatggaagagtgcgtatatcagacagcacagaattgatacaaactggagacgaggagaactcaaatctcctaaggtaacagactcttgacatactaacagaaacctttcttcaggaacaatgcctgctctcttcatgtgttggctgcagtgtaactcagtgcctatcttttatggaaaggaagcagctctatttcaaaattaactcagagtgttaagatcagtgttcttttatttctcatttctttaatctgataattttattgtaatcaaggatgagttgattcaggcatggtaccacatgcttgtagtccctgtattcaagtggccaaggcaggaggattactatgagttaaagccaacctgagcaatatactgagctctaggccaacgtgagctatagtatgaagccttgtctcaaaaatgtttctccccaaaaaggtgagttgagacagtgtgtgatactgtgctaccatgattctcactatgagtttgcagccttctagcctcacaggccaaatggatttttttttgtgtttgtttgtttttaatgatttgaagcagaagtctcattttctgtcttagaatattatttcagatgtgtaaatggtagaaatactaaatacatactttaataacaaaaagtgagccagctatgggaggcataattttatatgccagcatgccctgggataagttttaggttaaaaacaaaaaaccaaaaaaacaaaaaaccattatttgctattactctgtttcttggtgctttatttttcttctctggagtgctaaaagttggatctaaaacccactcttctcctccttttcctcttccacctctttctcttccttttcttctcctttactactgagctgaggttggctcagttatccaggctgccctggagctcaccttggtatcccaggcaggccttaaccctgggaccctctgttttgtcctccttgagaacaacagtcatcaggcctggttttcttctgtttttgtcaacagtatttactaaaataaagtaacttttcttttgacttaaacagattaggcatttgtgttagaaccatctttatgaagttttggcatagtaaaatgttaatagtgggcaaaggaatatggaaatatttaatgtttaatttatcatccaggaaaaagatgttgtgtcttatataaagttcattctaaaaatagattatagattactattctgacgcatggctccttcttgttttcactgtgtgtgtgtgtgatgtatgcacatgtgtgtgcacgcacacacacaggtcagaggaggtagtcagttttcctgttctcattctctgtctgacttactttttgagatagtctctctcactgatcctggagctaggctagcaattgtaagccccagcaacacgcccctcagtgtcagggttattgtgtgaccatacacagtttttctatgagttggagagatttggccctaggtcctgtatatctgagccatctctacagtatgtcactgtattctgaagaatgtgatatagtaacatccatgtgatctgtacacttttcttataggtgctgaaagggcatgatgaccatgtgatcacatgcctacagttttgtggcaaccgcatagttagtggttctgatgacaacactttaaaagtttggtcagcggtcacgggcaaggtaagttcactcacttctttctatatgtcagggagtcaccattttgtgtgtgtagtcacagtaaagtgggtgtttcttaatagcaaatatcaggcctcctttagaaacacgtcccttttcctgatagctatgagtgtcaccctggcatgctggcagcagtagggtttccttcatttaagtagctcgtgtctttgtcacttagtctaagatgtcccaggagcacatccttgttccttttcctcagcacctttcaccttgcctatgcgagttagaatcactacttcctaacgccttccaaaacatgacggagaaatgtgcaatttcccttatatgctgctttgttgctgagctgtctgcaatttagaaaccaggcacatcctgttaaggttgtcgcagacatcagtggtagggaatgaagcagggctgaagagccaagcagttgaagcagcggctgcaccgttcatacaccatggaccgttccttaaccagtctctagtgaatattgaattatatgttcaaaagagaacattttcatgaactaagtaattgaccatttataaaaataacacaagaaagtatgtgaaaagcttatgtttttagaaaatatgtcttgctgtgttgttccttggacaagagtcttacaaattctcaacttagatgttaaactgttcagagcagctatatatttccagggtataatagtagtttgggtttatctcggagtttcagcgtctgtctttacatgtctagaagtagcataggcagccctaactactgcctttaggcagatggggagagctgtggtatgtggccacacccaacgctccctgcattcttgccttccttctcgtcctggaagacaccctacagctcagcagtattttagtcctgcggccaagctagagttgtggaacttgcctgctcacagtatggttgcttcctggcatgtcttatgctttgtagcatatgattctctttaactttctttacttcattttattatatgcataagtgtttacttgtatgtatgtctgtgcaccacatgcatatcaggtgccttccaaggccagaacagggtgtcaggtccctgggcactagagttccaggtggttgggaggcaccatgtgcgtgccggagctcaaacccaggttcctgaaagagcaaccagtgctctaaatcactgagccatctccccagtcccttcctatgaatcttacggtttataccatgtttcctttctccatacttgttcctcagtcttgtatccctgtataagcatattctgctaccaaataagctaaaggtagtgatggtgtcagatagcattggtgtgcaaaaatgctttcctagtgcttcagtaaagttgaataagtaaaatttattccagattttaatgggatgttagtgtagtctttgacttacttgcatttctacctaaaattaatcatctgaagtgtttttccagtgtctgagaacgttagtgggacatacaggtggagtgtggtcatcacagatgagagacaatatcatcatcagtggatcgactgaccggactctcaaagtgtggaatgctgaaactggagagtgtatacatactttatatgggcacacttctactgtacggtgtatgcatctccatgaaaaaaggtaaaggatgtgccattatgattggggctcgcattactgttccttgtgactctgggattcatagagtgctaatgagctcttggagtggacactaaggatatagtgcagatggtccgaaattaagaaaattgggttatattggcttaaaagtggaggattgtctaaatttccttcttcatttgatatatggctgtttttcttcaaactctgctctctaatctgttttgatgttttgatgattcatcatctttaaacaaaaatcatttcctttcttgattttcgtattatgctgtgtaagaacaacaaattcagtcaccaggccttgttgcctgagtccttaagtgacacacccactctgcccccactccatcacatccctgcatatagacacagctaaggtccaggggccttaacttaatgtcagtactagtcatccaaactattgagagtgctcaaatatggggtcctcttgccttttgcacagctgacagagcgtcctcatattaggatggcttgctgccctacttctgcccaccaaatgaagggagaacttaaagtcccgttttcaaggtagtctcagccaagtctggcctgtttgccctttccttcccagttcctctgcctttatgtcaggcttctgctgacctcaggcccttgtgttgatacttcttcactgcacccagctaagaagttgtgttttctacttctttgtcacttttggtctgacgtgtcacacacatgctgctaatctttgcactgaaaaggtagacatgggtggatactgagttcccagcttttagaggagagccttcctctgacgctgtagcttagaagggtccttctgtggaatccacagcattcacgacctacagctctgtccagactgcttttcattaagaatcattgctgtaagcagaccttccctacagccatcattgtacagggactcctctcactcataaatttgagcctgctttgcacggtgtgttctgtgattaaaatttagggatgggtttcaaaggacaccacatttccgttttttgttgtgttcattctcccacatgtcagcactctgctctgtctaatgtagaatggcttttttatctcttgagaatgatgtaaaggtgcctggttagtaatctagcggtaactgcatgattgtctcgctctggttttcagaagagtatcattttatagcactgagacagttgctttaggcatttgtgttactgctataggaatatctttaagaatctctgagtcaccagctatgagcacgtgtctgaaactgactttccctgactgtgaagtataaggaaacaactatggctttctgagtcccaaattcactttaaaattctattttgttgagaaataattgtattgaaatgtctacttactcaaatagttaataggagttcataattgcagtgtagtgaaatgacaaagatcataaagaaggaaagttctggattgttgtaagttcataccacctggaggtgcattgaggatttatgcatatgccacatttccattttactgttaatcaggatcttgttgtgtgattggagagctgcttgcttcttgaatgcagtgctttccagactgtgccatggaaccttgaggtggaaccagggtccctaagtcagggaaccaatcagatggccagttgatctccaagtcttcctcacttcccattccaggcagaggagctggaatttgcatttgaaggcattaggtagagtttctcagaaaggatttctgtttaagaaaaagaggagaaagcaatggtttacctgtcctgtgcattcatttcctgactcttgtgttctgttctgctctgcagggttgtaagcggttctcgagatgccactctcagggtttgggatattgagaccggccagtgtttacacgtcctgatgggtcacgtagcagcggtccgctgcgttcagtatgatggcaggagggttgttagtggagcttatgattttatggtgaaggtgtgggatccagagactgagacctgtctacacacgttacagggacacactaatagagtctattcattacaggtaagaggccgtgtctctcccaccctttgattctctgccgttgactcataagattgttctcctcagttactgtcaagttgctgtttataagtaccttaaaattagcatgaagaaacacaaatttcaaatcatttaactgatttaatgatttaaatttctgtgatccattttctctttaaaatacaaatatttgtcttggaacctgatacttcaggattataaattttgctacactaattaatacttagataatctcataggagaataacttggattttaataaactccttagctgaggattaatttgaccaaaagctgcccctttgccttgatagctaaaagttgatggagcatgtaattgtgatttcatccccatttgaagtagctgtggaacctctctgcagaaagctctgtacttaacttgcccagagccactgactgggagccacagagagatgaatttgactaacctgttgttttcctttagatttgaacttgatatttattaatagactgtaatagttcaagatcttcagtaacaagacataaaacgtgggatacttttatttctggaaatgcagcattttccatcttaaaatatttcctgttacataatttccttcccagttactactgcctgttagtgatgagcaaagtagaacagaatcccagtgctgatgttgccgttcttagggcatttgattcttaattgatgatactgcccaagtaatgaagtattgatatataataaagatgttgattgcttcagaaaagaggatggcttccaaggggcgggaattggttgctaatgctgctgcttgtgatgtgctcagaactgtagagaatgatctcagaatagatagctgcatgtcatagagaccttctggggactggtagatgcttattggcttttagtgtccttgggaagctgaagaagagagactacatagattgcgtttgtttctaaaataccccctcttctaattcctgatacagttttcttttgggatatttgctaattagcattaatttcttgtaaacaacttcattttgcaaacactttttaatggaatttactattgaacatcatgaaagttatctgtggctattcccttgcctcaggtgctgataaacatgtctctgtctcaggcagtggctttggcttcctctagacaagtcaggctccatagtcctactaaatacctgggatgtagcttccagctgggaccaggtggtctgaaaaaaatgttgtttatttcaggtgctttgttgccttcttgaagttgtttctgccttgtttgaacgattcggctgttttgttttgtttctttttttaacttttaagagaatttatgaaagaagtattttgaagacatgatctatagactttcatggcatgcacatcagcttacctgcaagtttatcccagtaggctctttgttctggcctacgtgttgcttgtaccctctccctattgtgagtaatgtcaacagctgataggacaggaatccgtgtttctgttctaatactctgtgtctgttgtagtttgatggcatccatgtggtgagtggatctcttgatacatcaatccgagtctgggatgtggagacagggaattgtattcacacgctaacaggacaccagtcattaacgagtggaatggaactcaaagacaatattcttgtctctgggaatgcagattctacagttaagatctgggatatcaaaacaggacagtgtttacaaactttgcaaggtaagtcctggccctctgttagcctatctttaaaaacatcattgctattcaagagagaacctgtggagaacagaggtttaaccttatgcagagtacttactcctgatgagtttgtgaatgtttggctttcaaaagtaagccaccttgagaaatggttttcaattgtgtctttaatgtgaaaattcttaatcatttcctgtgtgttctgttgactctgtttaacatggctgtctcatagggcagcgttctttttgagtgatcattttattttgaattgaagcatctctgataggaaatgactttggccttgcttggtcaattccttcagtcggaatggaaggagtaatggtgctctctgaggggaaggtagagtgagaagcattagcattaggtatagcaccattggcttctccatgagtaacttccttctcttagtcacagcccttagaaggtgttgttgttgtttgtttgtttgtttgtttggagtgaagccatcaggaggcagcacatagcagcctttgttcattaattgtgactgttcctaggagtttggggtagtcacaaattctatttccttctttgactgatcagttcagtgtggcagagtgacagagtgacaaacctgcacgttcccttttccagcagtggaatgtgtacactggctgaccagggccactgggagccggagctcttgagacttggtactgtagttacatagccaccagatttccttacatctaagctggtcctaaactacagcaaggttgctaactgtgcagctttttaaatactgcatttcttgtatgccataatgtagaagagcacaagtcccacgcagaagccctgaacgtccttcctaagcagtaacccgcatttgcctttgcctaggtcccagcaagcatcagagcgctgtgacctgcttacagttcaacaagaacttcgtaattaccagctcagacgacggaacggtcaaactctgggacttgaaaacgggtgaatttatccgaaacctcgtcacattggagagtggggggagcgggggagttgtgtggcggatcagggcctcaaacacaaagctggtgtgtgcagtcgggagtcggaatggaactgaggaaaccaagctcctggtgctggactttgatgtggacatgaaatgaaaagcagacatgatgaattttgtccaactgtgtagacaatatactccctacccttccccctgcgcaaaaaacaaaaacaaacaaacaaaaaaatgaaaaaaaaaaacagaaaaaaaaaaagagaaaaaagaaaaggaaaaaaatcccttgtactcagtggtgcaggatgttggcttgggacaacagactgaaaagacctacagactaagaaggcaagaagagacaagagaccgtaactgacaggaggcggcagctgtcgcatctcgcaaaggcctcacttgtgactgaggggcagcttggcaagacgactctctaaatccaaccaggtgcaattattctttgttttcttctccagtggtcattgagcagagctacatcagcgttgttaccgtcacctagaaaggagtggcagtaatatccaaacacgggctgcttatcttctaatcagagcatctgcaacaaaccgtcatttttctgaagtggaaaagcttaaaacaattactgtgaattgtttttgtacagttatcatgaagctttcttttttctctttttcctgtttctcttcttttcttttttcttttttttttttgccaaccattgccaatgtcaatcaatcacagtattagcctctgttaatctatctctttactgttgcttctactcttcaatgcatatgttgctcaaaggtggcaagttgtcctgggttgtgtgagtcctgagatggatataattcttgatgctggtgctagaagtaggtcttcaaatccggggtcgttgtcccacccctgtactgtactcccagtggccaaacttatttatgctgctaaatgaaagaaagaaaaagcaaattattttttttttattttttttctgctgtgacgttttagtcccagactgaattccaaatttgctctagtttggttacagaaaaaaaagacttttttgccactgaaacttgagccatctgtgcctctaagaggctgagaatggaaaagtttcagataataaagagtgaagtttgcctgcaaataaagaattgagagtgtgtgcaaagcttattttcttttatctgggcaaaaattaaaacacattccttgggacagagcctgaggtgcctgttctgtggagaaacttctttttgagggctgtggtgaatggaagaacatacatagcaaaactgacaagatattttaaagatatataaaacacaaaggaaaaggaggttgctggtcagtcgtagcatcttacagtattggggaaaacaactgttacagtttcattgctctgagtgactgacgtgagaggaattcgctctgcagtgacgctgtctgtcactcgcctaccagctcgacgagcaagagagcgggagtcagatggtccgcctcattcaccaggagccgtaactcaagctgaactgtgaaagtggttaacactgtatcctaggccgtcttttttttcctcctcctgtttattttttgtttgttttatttatagtctgatttaaaacaatcagattcaagttggttaattttagttatgtaacaacctgacgtgatggaggaaacaacctgtaaagggattgtgtctatggtttgattcacttagaaattttattttcttataacttaagtgcaataaaatgtgttttttcatgtta

SEQ ID NO: 5 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 6 - protein, 214 aa, Mus musculus
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL

SEQ ID NO: 7 - DNA, 1420 bp, Homo sapiens
gataaagcgtgccaaggggcacacgacttgctgctcaggaaatccctgcggtctcaccgccgcgcctcgagagagagcgtgacagaggcctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggtaatatttggaggcctccgagggacgggcaggggaaagagggatcctctgacccagcgggggctgggaggatggctgtttttgttttttcccacctagcctcggaatcgcggactgcgcccagtgacggactcaaacttacccttccctctgaccccgccgtaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgaaaggacctgtctgtcgctgggctgtgggtgctaagggtaagggagagggagggagccgggagccgtagagggtggccgacggcggcggccctcaaaagcacttgttccttctgcttctccctggctgacccctggccggcccaggcctccacgggggcggcaggctgggttcattccccggccctccgagccgcgccaacgcacgcaacccttgctgctgcccgcgcgaagtgggcattgcaaagtgcgctcattttaggcctcctctctgccaccaccccataatctcattcaaagaatactagaatggtagcactacccggccggagccgcccaccgtcttgggtcgccctaccctcactca

SEQ ID NO: 8 - DNA, 1674 bp, Mus musculus
atgcagctcagaaatccctctgggtctcatcactgcagcagtggtcgagtacctcctcggagcttttctacgacttccagacgcaatttactccaggcgagggcgcctgcagtttagcagaacttcagagggagcagagaggctcagctatccactgctgcttgacactgaccctatccactgctgcttgtcactgactgacctgctgctctctattcttttgagtcgggagaactaggtaacaattcggaaactccaaagggtggatgaggggcgcgcggggtgtgtgtgggggatactctggtcccccgtgcagtgacctctaagtcagaggctggcacacacacaccttccattttttcccaaccgcaggatggcgcctcatcccttggatgcgctcaccatccaagtgtccccagagacacaacaaccttttcccggagcctcggaccacgaagtgctcagttccaattccaccccacctagccccactctcatacctagggactgctccgaagcagaagtgggtgactgccgagggacctcgaggaagctccgcgcccgacgcggagggcgcaacaggcccaagagcgagttggcactcagcaaacagcgaagaagccggcgcaagaaggccaatgatcgggagcgcaatcgcatgcacaacctcaactcggcgctggatgcgctgcgcggtgtcctgcccaccttcccggatgacgccaaacttacaaagatcgagaccctgcgcttcgcccacaactacatctgggcactgactcagacgctgcgcatagcggaccacagcttctatggcccggagccccctgtgccctgtggagagctggggagccccggaggtggctccaacggggactggggctctatctactccccagtctcccaagcgggtaacctgagccccacggcctcattggaggaattccctggcctgcaggtgcccagctccccatcctatctgctcccgggagcactggtgttctcagacttcttgtgaagagacctgtctggctctgggtggtgggtgctagtggaaagggaggggaccagagccgtctggagtgggaggtagtggaggctctcaagcatctcgcctcttctggctttcactacttggatccctagccctctcacagggcttaactaggcttctcatcggtacccttgctgctgcgcacagcagacattgggggctgctcttctcttaactctcctcggtgcagccacatcaaactctcgctccaagcatttgagaatggtagcactacctagttggagactcccatacttcctggtgagtctgccctcattcaaatctgccggcctccgaccatccatcactttttccagggtgacctaatccagtgttgcgtcttacctcactggctcctccatccagctcttggcccatagatgatgttcgtcgtctttactgcccgctacatgcagggtttctgagcttctccattctgccttagtccacgaaggtgatctgccttcttctgcacttttcaagtcgttacccttcccccaagggagaccaggctgtgaaccggaaagccctagcctatggctagagcatctctccaacttgtctcccgtgtctaaagtgtgagttgcagggacggttcctgaagcactgtttgtctccct

SEQ ID NO: 9 - DNA, 19 bp, Artificial sequence
  Synthetic sequence siRNA sequence
cttttggggacctcagagc

SEQ ID NO: 10 - DNA, 19 bp, Artificial sequence
  Synthetic sequence siRNA sequence
ccaacacaagtaaaacata

SEQ ID NO: 11 - DNA, 19 bp, Artificial sequence
  Synthetic sequence siRNA sequence
ccttctctggagagagaaa

SEQ ID NO: 12 - DNA, 19 bp, Artificial sequence
  Synthetic sequence siRNA sequence
agaaaccatgcaaagtctc

SEQ ID NO: 13 - DNA, 19 bp, Artificial sequence
  Synthetic sequence siRNA sequence
caagaactttgtaattacc

SEQ ID NO: 14 - DNA, 22 bp, Artificial sequence
  Synthetic sequence Primer
tgccagctgatataatggagaa

SEQ ID NO: 15 - DNA, 23 bp, Artificial sequence
  Synthetic sequence Primer
ccatgataggctttgatgacttt

SEQ ID NO: 16 - DNA, 27 bp, Artificial sequence
  Synthetic sequence Primer
cagagaggaggtactttggactataaa

SEQ ID NO: 17 - DNA, 21 bp, Artificial sequence
  Synthetic sequence Primer
gccatgttgaaacaatgacct

SEQ ID NO: 18 - DNA, 20 bp, Artificial sequence
  Synthetic sequence Primer
gaagtggaggacccacaagt

SEQ ID NO: 19 - DNA, 20 bp, Artificial sequence
  Synthetic sequence Primer
agtgccaaggtctgaaggtc

SEQ ID NO: 20 - DNA, 20 bp, Artificial sequence
  Synthetic sequence Primer
gaaatccaccaaagctcacg

SEQ ID NO: 21 - DNA, 18 bp, Artificial sequence
  Synthetic sequence Primer
cgggttccgctgtgtaag

SEQ ID NO: 22 - DNA, 17 bp, Artificial sequence
  Synthetic sequence Primer
cctgcgctcaggcaaaa

SEQ ID NO: 23 - DNA, 19 bp, Artificial sequence
  Synthetic sequence Primer
gctgggacaaacctttgca

SEQ ID NO: 24 - DNA, 17 bp, Artificial sequence
  Synthetic sequence Primer
gcgcctcatcccttgga

SEQ ID NO: 25 - DNA, 20 bp, Artificial sequence
  Synthetic sequence Primer
ccgggaaaaggttgttgtgt

SEQ ID NO: 26 - DNA, 20 bp, Artificial sequence
  Synthetic sequence pDEST-Rosa-NGN3-WT
ctgagccccacggcctcatt

SEQ ID NO: 27 - protein, 5 aa, Artificial sequence
  Synthetic Construct
SPTAS

SEQ ID NO: 28 - DNA, 20 bp, Artificial sequence
  Synthetic sequence pDEST-Rosa-NGN3-AA
ctggcccccacggccgcatt

SEQ ID NO: 29 - protein, 5 aa, Artificial sequence
  Synthetic Construct
APTAA

(19) United States
(12) Patent Application Publication
(10) Pub. No.: US 2015/0322406 A1
(43) Pub. Date: Nov. 12, 2015

(54) METHOD FOR REPROGRAMMING SOMATIC CELLS TO PANCREATIC BETA CELLS

(71) Applicant: CANCER RESEARCH TECHNOLOGY LIMITED, London (GB)

(72) Inventors: Axel BEHRENS, London (GB); Rocio SANCHO, London (GB)

(21) Appl. No.: 14/410,304

(22) PCT Filed: Jun. 24, 2013
(86) PCT No.: PCT/GB2013/051649

§ 371 Date: Dec. 22, 2014

(30) Foreign Application Priority Data
Jun. 29, 2012 (GB) 1211606.7

(51) Int. Cl. C12N 5/071; A61K 31/506; C12Q 1/25; A61K 35/39; C01D 15/04; A61K 31/519; A61K 31/4015; A61K 31/433; A61K 31/551; A61K 33/14; A61K 31/404

(52) U.S. Cl. CPC C12N 5/0676; A61K 33/14; A61K 31/506; C12Q 1/25; A61K 35/39; A61K 31/404; A61K 31/519; A61K 31/4015; A61K 31/433; A61K 31/551; C01D 15/04; C12N 2506/09; C12N 2506/22; C12N 2506/094; C12N 2501/999; G01N 2500/20; G01N 2333/9015

(57) ABSTRACT

The present invention relates to a method for reprogramming a somatic cell (e.g. a pancreatic ductal cell, a skin fibroblast or a keratinocyte) to a pancreatic β-cell. This method involves decreasing expression or activity of Fbw7 and/or decreasing phosphorylation-mediated degradation of NGN3 in the somatic cell. The method may be carried out ex vivo or in vivo. The invention also relates to a method of treating a subject with a metabolic disorder (e.g. diabetes) or a subject at risk of a metabolic disorder by administering somatic cells that have been re-programmed to pancreatic β-cells using methods described herein to the subject. In addition, the invention relates to screening methods for identifying a candidate agent for reprogramming a somatic cell to a pancreatic β-cell.

The present invention relates to a method for reprogramming a somatic cell, such as a pancreatic ductal cell, to a pancreatic β-cell. This method may be carried out ex vivo or in vivo. This method has therapeutic applications for the treatment of a subject with a metabolic disorder, such as diabetes, or a subject at risk of developing a metabolic disorder, such as diabetes.

The generation of specific therapeutically relevant cell types is a pre-requisite for cell replacement therapies and an important goal of personalized medicine. Pancreatic β-cells control glucose homeostasis through the production of insulin, and inadequate numbers of insulin-producing β-cells is the major cause of type 1 diabetes mellitus (reviewed in ¹). Therefore the replenishment of lost β-cells may allow for improved diabetes treatment².

The pancreas is composed of an exocrine (ductal and acinar cells) and an endocrine (β-cells, α-cells, δ-cells and pp cells) part, which are distinctively differentiated to secrete enzymes or hormones respectively⁵. Importantly, β-cells are not regenerated during adulthood. β-cell neogenesis in the adult has only been achieved experimentally with rather artificial approaches. For instance, pancreatic duct ligation⁶ and pancreatectomy⁷ experimentally induce adult β-cell neogenesis. Additionally, conversion of other cell types into β-cells by genetic approaches has been demonstrated. Adenoviral overexpression of the three transcription factors Ngn3, Maf1a and Pdx1 is sufficient to convert adult acinar cells into β-cells⁸, and extreme β-cell loss after diphtheria toxin overexpression or overexpression of Pax4 converts glucagon-producing cells into β-cells^{9,10}. However, the regulation and mechanisms of β-cell neogenesis in the normal adult pancreas remains largely unknown.

Fbw7 is the substrate recognition component of an SCF-type E3 ubiquitin ligase and can regulate the abundance of several transcription factor substrates involved in the control of progenitor cell fate decisions such as Notch1, c-Myc and c-Jun^{3,4}. Fbw7 is frequently inactivated by deletion, mutation or promoter hypermethylation in cancer²⁶. Fbw7 mutations occur in approximately 30% of T-cell acute lymphoblastic leukaemia (T-ALL), 16% of primary endometrial cancer and 11% of colorectal cancer. Overall, approximately 6% of all human primary tumours have mutations in Fbw7²⁷.

The present inventors have found that inactivation of fbw7 reprograms differentiated pancreatic ductal cells in adult mice into cells that show hallmarks of β-cells. The induced β-cells express proteins essential for β-cell function and are indistinguishable from islet β-cells in histological morphology and ultrastructure. Ductal cell to β-cell trans-differentiation occurred rapidly within several days, did not require cellular proliferation, and involved an intermediate stage when cells undergoing reprogramming co-expressed ductal and β-cell markers. Thus, inactivation of fbw7 induces direct cell reprogramming of exocrine ductal cells into β-cells without reversion to a progenitor cell state. Our study highlights the plasticity of seemingly differentiated adult cells, and identifies Fbw7 as a master regulator of cell fate decisions in the adult pancreas.

In a first aspect, the invention provides a method for reprogramming a somatic cell to a pancreatic β-cell, the method comprising the step of decreasing expression or activity of Fbw7 in the somatic cell. The somatic cell may, for example, be a pancreatic ductal cell, a skin fibroblast or a keratinocyte. Preferably, the somatic cell is a pancreatic ductal cell. The method may be carried out ex vivo or in vivo. For example, the method may be carried out in a subject with a metabolic disorder, such as diabetes, or in a subject at risk of developing a metabolic disorder, such as diabetes. The method may comprise contacting the somatic cell, e.g. pancreatic ductal cell, with an agent that reduces the expression or activity of Fbw7 in the somatic cell. The activity of Fbw7 may be the phosphorylation-mediated degradation of neurogenin-3 (NGN3). For example, the agent that reduces the expression or activity of Fbw7 may inhibit the phosphorylation of NGN3 by GSK3β.

In a second aspect, the invention provides a method for reprogramming a somatic cell to a pancreatic β-cell, the method comprising the step of decreasing phosphorylation-mediated degradation of NGN3. The somatic cell may, for example, be a pancreatic ductal cell, a skin fibroblast or a keratinocyte. Preferably, the somatic cell is a pancreatic ductal cell. In one embodiment, the method may comprise the step of decreasing phosphorylation of NGN3 by GSK3β. The method may be carried out ex vivo or in vivo. For example, the method may be carried out in a subject with a metabolic disorder, such as diabetes, or in a subject at risk of developing a metabolic disorder, such as diabetes. The method may comprise the step of contacting the somatic cell, e.g. pancreatic ductal cell, with an agent that decreases the phosphorylation of NGN3 by GSK3β, such as an inhibitor of GSK3β kinase activity.

In a third aspect, the invention provides an agent that reduces the expression or activity of Fbw7 for use in a method of reprogramming a somatic cell to a pancreatic β-cell, the method comprising the step of contacting the somatic cell with said agent. The somatic cell may, for example, be a pancreatic ductal cell, a skin fibroblast or a keratinocyte. Preferably, the somatic cell is a pancreatic ductal cell. The activity of Fbw7 may be the phosphorylation-mediated degradation of NGN3. For example, the agent may inhibit the phosphorylation of NGN3 by GSK3β.

In a fourth aspect, the invention provides an agent that decreases the phosphorylation-mediated degradation of NGN3 for use in a method of reprogramming a somatic cell to a pancreatic β-cell, the method comprising the step of contacting the somatic cell with said agent. The somatic cell may, for example, be a pancreatic ductal cell, a skin fibroblast or a keratinocyte. Preferably, the somatic cell is a pancreatic ductal cell. The agent may inhibit the phosphorylation of NGN3 by GSK3β. For example, the agent may be an inhibitor of GSK3β kinase activity.

In a fifth aspect, the invention provides a method of treating a subject with a metabolic disorder or a subject at risk of developing a metabolic disorder, the method comprising the step of administering to the subject pancreatic β-cells obtained by the method of the first or second aspect above. This method of treating a subject may include the step of reprogramming a somatic cell, such as a pancreatic ductal cell, to a pancreatic β-cell using the method of the first or second aspect above followed by the step of administering the pancreatic β-cells obtained by this method to the subject. The metabolic disorder is preferably diabetes.

In a sixth aspect, the invention provides pancreatic β-cells obtained by the method of the first or second aspect above for use in a method for treatment of a subject with a metabolic disorder or a subject at risk of developing a metabolic disorder, said method comprising administering said pancreatic β-cells to the subject. The metabolic disorder is preferably diabetes.

In a seventh aspect, the invention provides a screening method for identifying a candidate agent for reprogramming a somatic cell, such as a pancreatic ductal cell, to a pancreatic β-cell, the method comprising determining the ability of the agent to reduce expression or activity of Fbw7, wherein an agent that reduces expression or activity of Fbw7 is likely to be able to reprogram a somatic cell, such as a pancreatic ductal cell, to a pancreatic β-cell. The activity of Fbw7 may be the phosphorylation-mediated degradation of NGN3, for example the phosphorylation of NGN3 by GSK3β.

In an eighth aspect, the invention provides a screening method for identifying a candidate agent for reprogramming a somatic cell, such as a pancreatic ductal cell, to a pancreatic β-cell, the method comprising determining the ability of the agent to decrease phosphorylation-mediated degradation of NGN3, wherein an agent that decreases phosphorylation-mediated degradation of NGN3 is likely to be able to reprogram a somatic cell, such as a pancreatic ductal cell, to a pancreatic β-cell. The method may comprise the step of determining the ability of the agent to decrease phosphorylation of NGN3 by GSK3β, wherein an agent that decreases phosphorylation of NGN3 by GSK3β is likely to be able to reprogram a somatic cell, such as a pancreatic ductal cell, to a pancreatic β-cell.

These and other aspects of the invention are described in further detail below.

### BRIEF DESCRIPTION OF THE FIGURES

FIG. 1 shows that Pdx1 driven deletion of fbw7 in the pancreas induces occurrence of β-cells in the ducts containing functional and ultrastructural mature β-cell hallmarks (a-d) H&E staining of fbw7^{f/f} or Pdx1-Cre; fbw7^{f/f} pancreas. Numbered squares indicate fbw7^{f/f} pancreatic islet (1) or duct (2) or Pdx1-Cre; fbw7^{f/f} cells with altered morphology in the ducts (3,4). (e-g) Double immunofluorescence (IF) for glut2 and insulin (ins) in 4 week old fbw7^{f/f} (e) or Pdx1-Cre; fbw7^{f/f} (f-g) pancreas. Nuclei were counterstained with DAPI. Ducts are circled with a dashed line. Dashed square represents the area of the magnified picture (g). (h-j) Transmission electron micrograph of fbw7^{f/f} duct (h), fbw7^{f/f} islet β-cell (i) and an induced β-cell situated between two ductal cells in Pdx1-Cre; fbw7^{f/f} pancreas (j). Insets display a high magnification of the insulin granules. White arrows mark ductal cells, white asterisks mark β-cells, black asterisks mark duct lumen.

FIG. 2 shows that ductal β-cells formed after fbw7 deletion co-express different cell lineage markers. (a-b) Double immunofluorescence of fbw7^{f/f} (a) or Pdx1-Cre; fbw7^{f/f} (b) pancreas (4 weeks of age) for insulin (ins) and cytokeratin 19 (ck19). Nuclei were counterstained with DAPI. White dashed square represents the area of the adjacent magnified picture. (c-e) Triple immunofluorescence for glucagon (gluc), amylase (amy), and insulin (ins) in 4 week old fbw7^{f/f} (c) or Pdx1-Cre; fbw7^{f/f} (d-e) pancreas. Nuclei were counterstained with DAPI. White dashed square represents the area of the magnified picture.

FIG. 3 shows that inducible deletion of fbw7 in the adult pancreas promotes β-cell neogenesis from the duct. (a) Scheme of the mouse model. (b) Schematic diagram of RY pancreas before and after tamoxifen injection. (c) GFP immunoperoxidase staining in RY pancreas after tamoxifen injection. Section shows acinar cells, islet and a duct. (d) Quantification of GFP positive cells in the different pancreatic cell type compartments (A: acinar; I: Islet; D: Ducts) RY 13 days post-injection. (e-n) Triple immunofluorescence for insulin (ins), GFP (GFP), amylase (amy) in RY and RFY mice 13 days after tamoxifen injection. Nuclei were counterstained with DAPI. Ducts circled with a white line. White square represents the area of the magnified picture. (o) Western blot analysis of fbw7^{f/f} and Pdx1-Cre; fbw7^{f/f} pancreas lysates for Notch intracellular domain-1 (NICD1), phosphorylated c-jun at ser73 (p-c-jun^{ser73}), phosphorylated c-myc (p-c-myc) and phosphorylated cyclin-E (p-cyclin-E). (p-u) Double immunofluorescence of c-jun and p-c-jun^{ser73} in pancreas from fbw7^{f/f} and Pdx1-Cre; fbw7^{f/f} mice (4 weeks old). Ducts are circled with a white line.

FIG. 4 shows the results of intrapancreatic injection of 4-OH-tamoxifen in RY and RFY mice. (a) Schematic diagram of the experimental strategy. (b,d) GFP immuhistochemistry in RY 5 days (b) or 21 days (d) post-sugery. (c,e) GFP immuhistochemistry in RFY 5 days (c) or 21 days (e) post-surgery. (f,g) Quantification of GFP positive cells in the different pancreatic cell type compartments (A: acinar; I: Islet; D: Ducts) in RY and RFY 5 days (f) or 21 days (g) post-surgery. (h,i) Insulin immunostaining in RY and RFY 21 days post-surgery.

FIG. 5 shows intraperitoneal injection of tamoxifen in RY or RFY mice (a) Schematic diagram of the experimental strategy. (b-d) H&E staining of RY or RFY pancreas 12 weeks post tamoxifen injection. Arrows indicate altered morphology cells in the RFY ducts. (e-g) Insulin immunohistochemistry of RY or RFY pancreas 12 weeks post tamoxifen injection. Arrows indicate insulin positive cells in RFY ducts.

FIG. 6 Shows NGN3 phosphorylation is required for its interaction with Fbw7 and for its stability. (A) Schematic representation of the proposed mechanism of NGN3 degradation by Fbw7. (B) Western Blot analysis of overexpressed NGN3-HA in 293T cells upon GSK3 inhibitor. (C) Schematic representation of NGN3 GSK3 phosphorylation residues and the different NGN3 phospho-mutant construct generated. (D) NGN3-HA/Fbw7-Flag Immunoprecipitation assay using the different NGN3 constructs indicated in C. (E) Western Blot analysis of NGN3 in cells overexpressing the different NGN3 construct indicated in C.

FIG. 7 shows the proposed mechanism of action of Fbw7 in pancreatic progenitors. (A) In wild type situation, Fbw7 would promote the degradation of NGN3. (B) In the absence of Fbw7, the levels of NGN3 would increase.

FIG. 8 shows the proposed mechanism of action in a NGN3-AA conditional transgenic mouse. (A) In a wild type mouse NGN3 will be degraded normally by Fbw7 and the cells will remain as a progenitor status (B) In conditional NGN3-AA transgenic mouse, NGN3 will accumulate and induce differentiation of insulin producing cells.

FIG. 9 shows a schematic of the NGN3 and NGN3-AA constructs used to generate the conditional transgenic mice. (A) Schematic of the NGN3 and NGN3-AA intermediate construct (pDEST). (B) Western blot analysis of NGN3, HA, Flag and Myc in 293T transfected with pDEST-NGN3 or pDEST-NGN3-AA. (C) Schematic of the NGN3 and NGN3-AA constructs used to generate the conditional transgenic mice. (D) DNA sequencing of NGN3 and NGN3-AA constructs used to generate the conditional transgenic mice showing the phosphorylation sites.

FIG. 10 shows PCR analysis of unrecombined and recombined R26-LSL-NGN3-AA in Pdx1-Cre; R26-LSL-NGN3-AA double transgenic mouse. (A) Schematic representation of the locus before and after Cre-mediated recombination. The primers used in the PCR are indicated with arrows. (B) PCR in genomic DNA from tail, liver and pancreas using primers 1,2 and 3. R26-Δ-NGN3-AA: recombined allele; R26-LSL-NGN3: unrecombined allele.

FIG. 11 shows expression of NGN3-AA in the pancreas of the NGN3-AA transgenic mouse. Western blot analysis of NGN3 in Pancreas and liver lysates from Pdx1-Cre; R26-LSL-NGN3-AA and R26-LSL-NGN3-AA mouse.

FIG. 12 shows Q-PCR results for different genes involved in beta cell differentiation in Pdx1-Cre; R26-LSL-NGN3-AA and R26-LSL-NGN3-AA mouse liver and pancreas. (A) Ins1 (B) neurod1 (C) pdx1 (D) ins2 (E) ngn3 (F) hes1.

FIG. 13 shows a low magnification of insulin immunostaining in (A) Pdx1-Cre; R26-LSL-NGN3-AA and (B) R26-LSL-NGN3-AA pancreas.

FIG. 14 shows pancreatic spheres generated in Fbw7+/+; Rosa-CreERT and Fbw7 F/F; Rosa-CreERT mice injected with tamoxifen. (A) Day 4 after culture (B) Day 15 after culture.

FIG. 15 shows YFP fluorescence of pancreatic spheres generated in Fbw7+/+; Rosa-CreERT and Fbw7 F/F; Rosa-CreERT mice injected with tamoxifen after 14 days in 24 well adherent plates (A) Bright field (B) YFP fluorescence (C) High magnification of spheres.

FIG. 16 shows pancreatic spheres and differentiated cells generated in Fbw7+/+; Rosa-CreERT and Fbw7 F/F; Rosa-CreERT mice injected with tamoxifen after dissociation of day 4 spheres (A) Fbw7+/+; Rosa-CreERT (B) Fbw7 F/F; Rosa-CreERT low and high mag of spheres and differentiated cells.

FIG. 17 shows insulin and CK19 staining in Fbw7 F/F; Rosa-CreERT differentiated cells. (A) Bright field and Insulin immunofluorescence. (B) Insulin/DAPI and (C) CK19/DAPI staining.

FIG. 18 shows that converted β-cells induced by fbw7 deletion show hallmarks of functional mature β-cells. (a) Schematic diagram of the comparison strategy used for the mRNA expression profile. Heat map representation of 15 representative β-cell markers in GFP+ sorted cells from tamoxifen injected CY, CFY and MIP-GFP mice. (b) qPCR analysis of ins2, gck, pdx1 and nkx6.1 in GFP+ cells sorted from CY and CFY cells. (c) Double immunofluorescence of ductal CFY β-cells for insulin (ins) together with different β-cell markers (c-PPT, glut2, MafA1, Nkx6.1, Pax6, PC1/3, Pdx1) or ductal markers (Sox9, DBA) (scale bar: 5 μm). (d) Schematic diagram of the glucose challenge experiment design and FACS profile in uncompensated signal, indicating the sorting gating used for the isolation of the GFP+ cells in CY and CFY mice. ELISA for insulin levels in the supernatant from GFP+ cells from CY and CFY 0, 15, 30 and 60 min after the addition of 20 mM glucose.

FIG. 19 shows the blood glucose level in Pdx1-Cre, R26-LSL-NGN3-AA and R26-LSL-NGN3-AA mice after intraperitoneal injection of glucose (2 g/kg bw).

### DETAILED DESCRIPTION

The present invention is based on the finding that inactivation of fbw7 reprograms differentiated somatic cells, e.g. pancreatic ductal cells, into cells that show hallmarks of pancreatic β-cells. The invention is also based on the identification of NGN3 as a novel substrate for Fbw7 and the finding that Fbw7 targets NGN3 for ubiquitin-mediated destruction by the proteasome.

In a first aspect, the invention provides a method for reprogramming a somatic cell to a pancreatic β-cell, the method comprising the step of decreasing expression or activity of Fbw7 in the somatic cell. The somatic cell may, for example, be a pancreatic ductal cell, a skin fibroblast or a keratinocyte. Preferably, the somatic cell is a pancreatic ductal cell. The method may be carried out ex vivo or in vivo. For example, the method may be carried out in a subject with a metabolic disorder, such as diabetes, or in a subject at risk of developing a metabolic disorder, such as diabetes. Alternatively, the method may be carried out in an experimental animal, such as a mouse or rat.

The method may comprise contacting the somatic cell, such as a pancreatic ductal cell, with an agent that reduces the expression or activity of Fbw7 in the somatic cell. The activity of Fbw7 may be the phosphorylation-mediated degradation of NGN3. For example, the agent that reduces the expression or activity of Fbw7 may inhibit the phosphorylation of NGN3 by GSK3β.

Pancreatic ductal cells form the epithelial lining of the branched tubes that deliver enzymes produced by pancreatic acinar cells into the duodenum. In addition, these cells secrete bicarbonate that neutralizes stomach acidity. During development, epithelium of endodermal origin evaginates from the future duodenum area and invades the mesenchyme to form a complex branched network. All endocrine, acinar and ductal cells arise from common precursors in this epithelial structure. Adult ductal cells share some similarities with embryonic primitive ducts and may retain the ability to generate endocrine cells in the adult²⁸. Pancreatic ductal cells may, for example, be obtained from pancreatic biopsies, e.g. from a human pancreatic biopsy, or from a full post-mortem pancreas in the case of an experimental animal. Pancreatic tissue may, for example, be digested using collagenase and single cell suspensions of pancreatic ductal cells cultured as described in detail below.

The pancreatic ductal cells have preferably been obtained from a subject with a metabolic disorder, such as diabetes, or from a subject at risk of developing a metabolic disorder, such as diabetes. For example, these cells may have been obtained from a pancreatic biopsy taken from a subject with a metabolic disorder, such as diabetes, or from a subject at risk of developing a metabolic disorder, such as diabetes. The subject is preferably a mammal, and most preferably a human.

Fbw7 is an ubiquitin E3 ligase substrate adaptor. Ubiquitin E3 ligases can physically bind to and add ubiquitin chains to a target protein to result in its recognition and degradation in an ATP-dependent manner by the proteasome. Fbw7 (or FBW7) is also known as F-box/WD repeat-containing protein 7 isoform 1, CDC4, Ago (or AGO), hSel10, Fbxw7, FLJ16457, FBW6, SEL-10, CDC4, DKFZp686F23254, FBX30, FBXO30, FBXW6, or SEL1.

The amino acid and nucleotide sequences of human and mouse Fbw7 are shown in Tables 1, 2a and 2b below.

When carried out ex vivo, the method may comprise the step of decreasing expression or activity of Fbw7 in a somatic cell, such as a pancreatic ductal cell, obtained from a subject with a metabolic disorder, such as diabetes, or from a subject at risk of developing a metabolic disorder, such as diabetes. The method may comprise contacting the somatic cell, such as a pancreatic ductal cell, with an agent that reduces the expression or activity of Fbw7 in the somatic cell. The activity of Fbw7 may be the phosphorylation-mediated degradation of NGN3. For example, the agent that reduces the expression or activity of Fbw7 may inhibit the phosphorylation of NGN3 by GSK3β.

When carried out in vivo, the method may comprise the step of decreasing expression or activity of Fbw7 in a somatic cell, such as a pancreatic ductal cell, present in a subject with a metabolic disorder, such as diabetes, in a subject at risk of developing a metabolic disorder, such as diabetes, or in an experimental animal, such as a mouse or a rat. For example, the method may comprise the step of administering to the subject or experimental animal an agent that reduces the expression or activity of Fbw7 in a somatic cell, such as a pancreatic ductal cell. The activity of Fbw7 may be the phosphorylation-mediated degradation of NGN3. For example, the agent that reduces the expression or activity of Fbw7 may inhibit the phosphorylation of NGN3 by GSK3β. The subject is preferably a mammal, and most preferably a human.

The agent that reduces the expression or activity of Fbw7 may be administered to the subject or experimental animal by any suitable route of administration. For example, the agent may be administered by intraductal, intraperitoneal or intravenous injection or orally.

Decreased or reduced expression of Fbw7 may be achieved at the nucleic acid level or at the protein level. For example, the transcription of the Fbw7 gene into Fbw7 mRNA may be decreased or reduced, or the translation of Fbw7 mRNA into Fbw7 protein may be decreased or reduced.

Alternatively, decreased or reduced Fbw7 expression or activity may be achieved by decreasing or reducing Fbw7 protein stability or by inhibiting Fbw7 dimerisation.

The activity of Fbw7 includes its downstream effects on its substrates such as c-Myc, Cyclin E, c-jun, c-Myb, KLF5, Mcl-1, mTOR, Notch1, SREBP1, presenilin and Notch intracellular domain 1 (NICD1). The inventors have also identified NGN3 as a novel substrate for Fbw7 and have found that Fbw7 targets NGN3 for ubiquitin-mediated destruction by the proteasome, thus reducing its level in cells. Specifically, Fbw7 recognises phosphorylated NGN3 and targets it for ubiquitin-mediated destruction. Therefore, reducing the activity of Fbw7 may include reducing its downstream activity, e.g. by reducing ubiquitin-mediated destruction of NGN3 by Fbw7. For example, reducing the activity of Fbw7 may include reducing the phosphorylation of NGN3. This reduces its targeting for ubiquitin-mediated destruction by Fbw7, thus reducing the downstream activity of Fbw7. For example, the agent that reduces the expression or activity of Fbw7 may inhibit the phosphorylation of NGN3, e.g. by GSK3β. Therefore, the agent that reduces the expression or activity of Fbw7 may be an inhibitor of GSK3β. Examples of agents that reduces the expression or activity of Fbw7 by inhibiting the phosphorylation of NGN3 by GSK3β include lithium chloride, CHIR-99021 (CT99021), SB 216763, TWS119, CHIR-98014, SB 415286, tideglusib, LY2090314 and TDZD-8.

NGN3 (neurogenin-3) is a regulator of pancreatic islet differentiation and regeneration²⁹. NGN3 is also known as Neurog3, Atoh5, MGC129292, MGC129293, Math4B, bHLHa7, Atonal protein homolog 5, Helix-loop-helix protein mATH-4B, MATH4B), atonal homolog 5, protein atonal homolog 5.

The amino acid and nucleotide sequences of human and mouse NGN3 are shown in Tables 3 and 4 below.

The agent that reduces the expression or activity of Fbw7 for use in the invention may, for example, be a small molecule, a nucleotide sequence, a protein (e.g. an antibody), a peptide, a modified peptide (e.g. a stapled peptide), an aptamer or an RNA interference (RNAi) agent (e.g. an siRNA). For example, the agent may be a small molecule inhibitor of NGN3 phosphorylation by GSK3β (as discussed above), e.g. lithium chloride, CHIR-99021 (CT99021), SB 216763, TWS119, CHIR-98014, SB 415286, tideglusib, LY2090314 or TDZD-8; an anti-Fbw7 antibody; or an siRNA molecule that reduces the expression of Fbw7. The agent may also reduce or decrease the stability of Fbw7 or inhibit its dimerization. Exemplary siRNA sequences that can be used to reduce the expression of Fbw7 include:

| | |
|---|---|
| (a) | 5′-cttttggggacctcagagc-3′ |
| (b) | 5′-ccaacacaagtaaaacata-3′ |
| (c) | 5′-ccttctctggagagagaaa-3′ |
| (d) | 5′-agaaaccatgcaaagtctc-3′ |
| (e) | 5′-caagaactttgtaattacc-3′ |

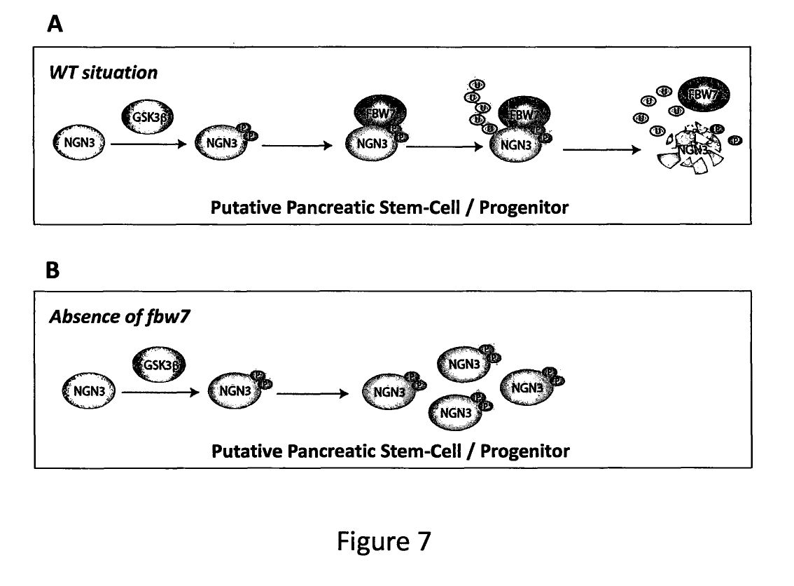

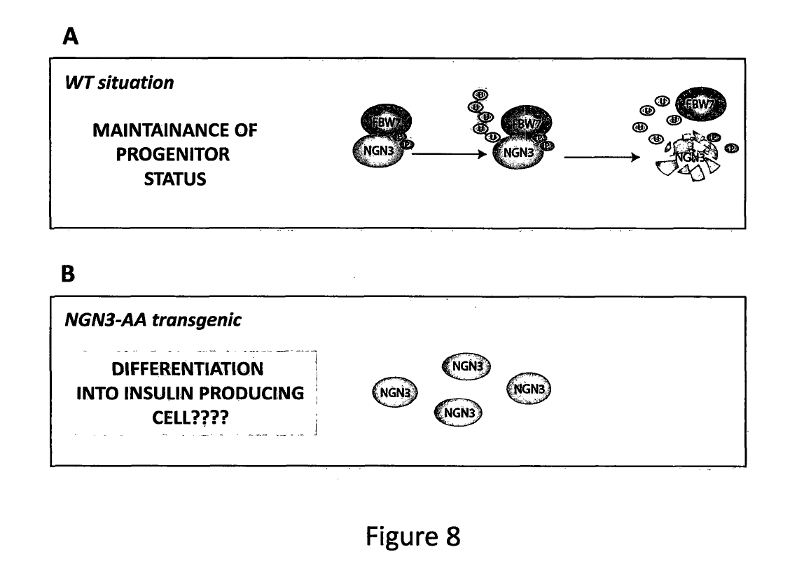

As mentioned above and as demonstrated by the Examples below, the inventors have identified NGN3 as a novel substrate for Fbw7 and have found that Fbw7 targets NGN3 for ubiquitin-mediated destruction by the proteasome, thus reducing its level in cells. Specifically, Fbw7 recognises phosphorylated NGN3 and targets it for ubiquitin-mediated destruction (see FIGS. 7 and 8).

Therefore, in a second aspect, the invention provides a method for reprogramming a pancreatic ductal cell to a pancreatic β-cell, the method comprising the step of decreasing phosphorylation-mediated degradation of NGN3. For example, the method may comprise the step of decreasing phosphorylation of NGN3 by GSK3β. The method may be carried out ex vivo or in vivo. For example, the method may be carried out in a subject with a metabolic disorder, such as diabetes, in a subject at risk of developing a metabolic disorder, such as diabetes, or in an experimental animal. The method may comprise the step of contacting the somatic cell, such as a pancreatic ductal cell, with an agent that decreases the phosphorylation of NGN3 by GSK3β, such as an inhibitor of GSK3β kinase activity.

The pancreatic ductal cells have preferably been obtained from a subject with a metabolic disorder, such as diabetes, or from a subject at risk of developing a metabolic disorder, such as diabetes. For example, these cells may have been obtained from a pancreatic biopsy taken from a subject with a metabolic disorder, such as diabetes, or from a subject at risk of developing a metabolic disorder, such as diabetes. The subject is preferably a mammal, and most preferably a human.

When carried out ex vivo, the method may comprise the step of decreasing phosphorylation-mediated degradation of NGN3 in a somatic cell, such as a pancreatic ductal cell, obtained from a subject with a metabolic disorder, such as diabetes, or from a subject at risk of developing a metabolic disorder, such as diabetes. The method may comprise contacting the somatic cell, such as a pancreatic ductal cell, with an agent that decreases the phosphorylation-mediated degradation of NGN3 in the somatic cell. For example, the agent that decreases phosphorylation-mediated degradation of NGN3 may inhibit phosphorylation of NGN3 by GSK3β. The subject is preferably a mammal, and most preferably a human.

When carried out in vivo, the method may comprise the step of decreasing phosphorylation-mediated degradation of NGN3 in a somatic cell, such as a pancreatic ductal cell, present in a subject with a metabolic disorder, such as diabetes, in a subject at risk of developing a metabolic disorder, such as diabetes, or in an experimental animal, such as a mouse or rat. For example, the method may comprise the step of administering to the subject or experimental animal an agent that decreases the phosphorylation-mediated degradation of NGN3 in the somatic cell, such as a pancreatic ductal cell. For example, the agent that decreases phosphorylation-mediated degradation of NGN3 may inhibit phosphorylation of NGN3 by GSK3β. The subject is preferably a mammal, and most preferably a human.

The agent that decreases the phosphorylation-mediated degradation of NGN3 for use in the invention may, for example, be a small molecule, a nucleotide sequence, a protein (e.g. an antibody), a peptide, a modified peptide (e.g. a stapled peptide), an aptamer or an RNA interference (RNAi) agent (e.g. an siRNA). For example, the agent may be a small molecule inhibitor of NGN3 phosphorylation by GSK3β (e.g. lithium chloride, CHIR-99021 (CT99021), SB 216763, TWS119, CHIR-98014, SB 415286, tideglusib, LY2090314 or TDZD-8), an anti-GSK3β antibody or an siRNA molecule that reduces the expression of GSK3β.

In a third aspect, the invention provides an agent that reduces the expression or activity of Fbw7 for use in a method of reprogramming a somatic cell to a pancreatic β-cell, the method comprising the step of contacting the somatic cell with said agent. For example, the somatic cell may be a pancreatic ductal cell, a skin fibroblast or a keratinocyte. The somatic cell is preferably a pancreatic ductal cell. The activity of Fbw7 may be the phosphorylation-mediated degradation of NGN3. For example, the agent may inhibit the phosphorylation of NGN3 by GSK3β.

The pancreatic ductal cells have preferably been obtained from a subject with a metabolic disorder, such as diabetes, or from a subject at risk of developing a metabolic disorder, such as diabetes. For example, these cells may have been obtained from a pancreatic biopsy taken from a subject with a metabolic disorder, such as diabetes, or from a subject at risk of developing a metabolic disorder, such as diabetes. The subject is preferably a mammal, and most preferably a human.

As set out above, Fbw7 (or FBW7) is also known as F-box/WD repeat-containing protein 7 isoform 1, CDC4, Ago (or AGO), hSel10, Fbxw7, FLJ16457, FBW6, SEL-10, CDC4, DKFZp686F23254, FBX30, FBXO30, FBXW6, or SEL1. The amino acid and nucleotide sequences of human and mouse Fbw7 are shown in Table 1.

The method of reprogramming the somatic cell, such as a pancreatic ductal cell, may be carried out ex vivo or in vivo. When the method is carried out ex vivo, the method may include the step of obtaining a somatic cell, such as a pancreatic ductal cell, from a subject with a metabolic disorder, such as diabetes, or from a subject at risk of developing a metabolic disorder, such as diabetes, and then contacting the somatic cell, such as a pancreatic ductal cell, with said agent ex vivo. When the method is carried out in vivo, the method may include the step of contacting a somatic cell, such as a pancreatic ductal cell, present in a subject with a metabolic disorder, such as diabetes, or in a subject at risk of developing a metabolic disorder, such as diabetes, with said agent. For example, the method may include administering said agent to a subject with a metabolic disorder, such as diabetes, or in a subject at risk of developing a metabolic disorder, such as diabetes. The subject is preferably a mammal, and most preferably a human.

Decreased or reduced expression of Fbw7 may be achieved at the nucleic acid level or at the protein level. For example, transcription of the Fbw7 gene into Fbw7 mRNA may be decreased or reduced, or translation of Fbw7 mRNA into Fbw7 protein may be decreased or reduced.

Alternatively, decreased or reduced Fbw7 expression or activity may be achieved by decreasing or reducing Fbw7 protein stability or by inhibiting Fbw7 dimerisation.

The activity of Fbw7 includes its downstream effects on its substrates such as c-Myc, Cyclin E, c-jun, c-Myb, KLF5, Mcl-1, mTOR, Notch1, SREBP1 and presenilin. As described herein, the inventors have also identified NGN3 as a novel substrate for Fbw7 and have found that Fbw7 targets NGN3 for ubiquitin-mediated destruction by the proteasome, thus reducing its level in cells. Specifically, Fbw7 recognises phosphorylated NGN3 and targets it for ubiquitin-mediated destruction. Therefore, reducing the activity of Fbw7 includes reducing its downstream activity, e.g. by reducing ubiquitin-mediated destruction of NGN3 by Fbw7. For example, reducing the activity of Fbw7 may include reducing the phosphorylation of NGN3. This reduces its targeting for ubiquitin-mediated destruction by Fbw7, thus reducing the downstream activity of Fbw7. For example, the agent that reduces the expression or activity of Fbw7 may inhibit the phosphorylation of NGN3, e.g. by GSK3β. Therefore, the agent that reduces the expression or activity of Fbw7 may be an inhibitor of GSK3β. Examples of agents that reduce the expression or activity of Fbw7 by inhibiting the phosphorylation of NGN3 by GSK3β include lithium chloride, CHIR-99021 (CT99021), SB 216763, TWS119, CHIR-98014, SB 415286, tideglusib, LY2090314 and TDZD-8.

The agent that reduces the expression or activity of Fbw7 for use in the invention may, for example, be a small molecule, a nucleotide sequence, a protein (e.g. an antibody), a peptide, a modified peptide (e.g. a stapled peptide), an aptamer or an RNA interference (RNAi) agent (e.g. an siRNA). For example, the agent may be a small molecule inhibitor of NGN3 phosphorylation by GSK3β (as discussed above), e.g. lithium chloride, CHIR-99021 (CT99021), SB 216763, TWS119, CHIR-98014, SB 415286, tideglusib, LY2090314 or TDZD-8; an anti-Fbw7 antibody; an anti-Fbw7 antibody; or an siRNA molecule that reduces the expression of Fbw7. Exemplary siRNA sequences that can be used to reduce the expression of Fbw7 include:

| | |
|---|---|
| (a) | 5′-cttttggggacctcagagc-3′ |
| (b) | 5′-ccaacacaagtaaaacata-3′ |
| (c) | 5′-ccttctctggagagagaaa-3′ |
| (d) | 5′-agaaaccatgcaaagtctc-3′ |
| (e) | 5′-caagaactttgtaattacc-3′ |

In a fourth aspect, the invention provides an agent that decreases the phosphorylation-mediated degradation of NGN3 for use in a method of reprogramming a pancreatic ductal cell to a pancreatic β-cell, the method comprising the step of contacting the pancreatic ductal cell with said agent. The agent may inhibit the phosphorylation of NGN3 by GSK3β. For example, the agent may be an inhibitor of GSK3β kinase activity.

The somatic cells, such as pancreatic ductal cells, have preferably been obtained from a subject with a metabolic disorder, such as diabetes, or from a subject at risk of developing a metabolic disorder, such as diabetes. For example, these cells may have been obtained from a pancreatic biopsy taken from a subject with a metabolic disorder, such as diabetes, or from a subject at risk of developing a metabolic disorder, such as diabetes.

As set out above, NGN3 is also known as Neurog3, Atoh5, MGC129292, MGC129293, Math4B, bHLHa7, Atonal protein homolog 5, Helix-loop-helix protein mATH-4B, MATH4B), atonal homolog 5, protein atonal homolog 5.

The amino acid and nucleotide sequences of human and mouse NGN3 are shown in Tables 3 and 4.

The method of reprogramming the somatic cell, such as a pancreatic ductal cell, may be carried out ex vivo or in vivo. When the method is carried out ex vivo, the method may include the step of obtaining a somatic cell, such as a pancreatic ductal cell, from a subject with a metabolic disorder, such as diabetes, or from a subject at risk of developing a metabolic disorder, such as diabetes, and then contacting the somatic cell, such as a pancreatic ductal cell, with said agent ex vivo. The subject is preferably a mammal, and most preferably a human. When the method is carried out in vivo, the method may include the step of contacting a somatic cell, such as a pancreatic ductal cell, present in a subject with a metabolic disorder, such as diabetes, or in a subject at risk of developing a metabolic disorder, such as diabetes, with said agent. For example, the method may include administering said agent to a subject with a metabolic disorder, such as diabetes, or in a subject at risk of developing a metabolic disorder, such as diabetes. The subject is preferably a mammal, and most preferably a human.

The agent that decreases the phosphorylation-mediated degradation of NGN3 for use in the invention may, for example, be a small molecule, a nucleotide sequence, a protein (e.g. an antibody), a peptide, a modified peptide (e.g. a stapled peptide), an aptamer or an RNA interference (RNAi) agent (e.g. an siRNA). For example, the agent may be a small molecule inhibitor of NGN3 phosphorylation by GSK3β (as discussed above), e.g. lithium chloride, an anti-GSK3β antibody or an siRNA molecule that reduces the expression of GSK3β. Exemplary siRNA sequences that can be used to reduce the expression of Fbw7 include:

| | |
|---|---|
| (a) | 5′-cttttggggacctcagagc-3′ |
| (b) | 5′-ccaacacaagtaaaacata-3′ |
| (c) | 5′-ccttctctggagagagaaa-3′ |
| (d) | 5′-agaaaccatgcaaagtctc-3′ |
| (e) | 5′-caagaactttgtaattacc-3′ |

In a fifth aspect, the invention provides a method of treating a subject with a metabolic disorder or a subject at risk of developing a metabolic disorder, the method comprising the step of administering to the subject pancreatic β-cells obtained by the method of the first or second aspect above. This method of treating a subject may include the step of reprogramming a somatic cell, such as a pancreatic ductal cell, to a pancreatic β-cell using the method of the first or second aspect above followed by the step of administering the pancreatic β-cells obtained by this method to the subject. The metabolic disorder is preferably diabetes. The subject is preferably a mammalian subject and most preferably a human subject. Pancreatic β-cells may be administered to the subject by any suitable method, such as intra-portal vein (liver) islet transplantation or kidney capsule transplantation in humans, and kidney capsule, intrapancreatic or intrahepatic transplantation in mice.

In a sixth aspect, the invention provides pancreatic β-cells obtained by the method of the first or second aspect above for use in a method for treatment of a subject with a metabolic disorder or a subject at risk of developing a metabolic disorder, said method comprising administering said pancreatic β-cells to the subject. The metabolic disorder is preferably diabetes. The subject is preferably a mammalian subject and most preferably a human subject. Pancreatic β-cells may be administered to the subject by any suitable method, such as intra-portal vein (liver) islet transplantation or kidney capsule transplantation in humans, and kidney capsule, intrapancreatic or intrahepatic transplantation in mice.

In a seventh aspect, the invention provides a screening method for identifying a candidate agent for reprogramming a somatic cell to a pancreatic β-cell, the method comprising determining the ability of the agent to reduce expression or activity of Fbw7, wherein an agent that reduces expression or activity of Fbw7 is likely to be able to reprogram the somatic cell to a pancreatic β-cell. For example, the somatic cell may be a pancreatic ductal cell, a skin fibroblast or a keratinocyte. The somatic cell is preferably a pancreatic ductal cell. The activity of Fbw7 may be the phosphorylation-mediated degradation of NGN3, for example the phosphorylation of NGN3 by GSK33.

The screening method may be carried out in vitro or in vivo.

The candidate agent may reduce expression of Fbw7 at the nucleic acid level or at the protein level. For example, the candidate agent may reduce transcription of the Fbw7 gene into Fbw7 mRNA or the candidate agent may reduce translation of Fbw7 mRNA into Fbw7 protein may be reduced.

The activity of Fbw7 includes its downstream effects on its substrates such as c-Myc, Cyclin E, c-jun, c-Myb, KLF5, Mcl-1, mTOR, Notch1, SREBP1 and presenilin. As described herein, reducing the activity of Fbw7 may include reducing ubiquitin-mediated destruction of NGN3 by Fbw7. For example, reducing the activity of Fbw7 may include reducing its downstream activity, e.g. by reducing the phosphorylation of NGN3. This reduces its targeting for ubiquitin-mediated destruction by Fbw7, thus reducing the downstream activity of Fbw7. For example, the method may comprise determining the ability of the agent to reduce phosphorylation of NGN3 (e.g. by GSK3β), wherein an agent that reduces phosphorylation of NGN3 is likely to be able to reprogram a somatic cell, such as a pancreatic ductal cell, to a pancreatic β-cell.

The method may comprise the step of contacting a cell expressing Fbw7 with the candidate agent and determining the level of Fbw7 mRNA in the cell before and after the contacting step, wherein a decrease in Fbw7 mRNA following the contacting step indicates that the agent is likely to be able to reprogram a somatic cell, such as a pancreatic ductal cell, to a pancreatic β-cell. The level of Fbw7 mRNA may be determined by any suitable method known in the art, such as quantitative PCR.

The method may comprise the step of contacting a cell co-expressing NGN3 and Fbw7 with the candidate agent and determining ubiquitination of NGN3 by Fbw7 in the absence or presence of the agent, wherein a reduction in ubiquitination of NGN3 following the step of contacting the cell with the agent indicated that the agent is likely to be able to reprogram a somatic cell, such as a pancreatic ductal cell, to a pancreatic β-cell.

The method may comprise the step of contacting a cell expressing Fbw7 with the candidate agent and determining the level of Fbw7 protein expression or expression of a substrate of Fbw7, such as NGN3, c-Jun or Notch intracellular domain 1 (NICD-1), in the cell before and after the contacting step, wherein a decrease in Fbw7 protein expression or expression of a substrate of Fbw7 following the contacting step indicates that the agent is likely to be able to reprogram a somatic cell, such as a pancreatic ductal cell, to a pancreatic β-cell. The level of Fbw7 protein or a substrate of FBw7 may be determined by any suitable method known in the art, such as immunohistochemistry.

In an eighth aspect, the invention provides a screening method for identifying a candidate agent for reprogramming a somatic cell to a pancreatic β-cell, the method comprising determining the ability of the agent to decrease phosphorylation-mediated degradation of NGN3, wherein an agent that decreases phosphorylation-mediated degradation of NGN3 is likely to be able to reprogram a somatic cell to a pancreatic β-cell. For example, the somatic cell may be a pancreatic ductal cell, a skin fibroblast or a keratinocyte. The somatic cell is preferably a pancreatic ductal cell. The method may comprise the step of determining the ability of the agent to decrease phosphorylation of NGN3 by GSK3β, wherein an agent that decreases phosphorylation of NGN3 by GSK3β is likely to be able to reprogram a somatic cell, such as a pancreatic ductal cell, to a pancreatic β-cell.

The step of determining whether an agent decreases phosphorylation of NGN3 by GSK3β may be carried out by any suitable method known in the art, such as phospho-specific NGN3 antibody development. Phosphorylation of NGN3 may also be determined indirectly by determining its ability to interact with Fbw7, wherein an agent that decreases the ability of NGN3 to interact with Fbw7 is likely to be able to reprogram a somatic cell, such as a pancreatic ductal cell, to a pancreatic β-cell.

Further aspects and embodiments of the invention will be apparent to those skilled in the art given the present disclosure including the following experimental exemplification.

### Experimental

### Materials and Methods

### Mouse Lines

The Pdx1-cre²¹, R26-LSL-YFP²², R26-CreERT²³ and Fbw7^{f/f 24} mouse lines have been previously described.

### Genetic Labelling Experiments

Mice were either injected intraperitoneally with tamoxifen (TM) or intrapancreatically injected with 4-OH-tamoxifen as indicated. Short and long term histological analyses were performed at short. (5 or 13 days) or long (60 or 82 days) post-injections. Pancreata were excised, fixed in cold 4% paraformaldehyde for 12 h, embedded in paraffin, cut into 7-10 μm sections and staining was performed as described below.

### Immunofluorescence Staining

Mice were euthanized by cervical dislocation and pancreas dissected out into ice-cold PBS. The pancreas was fixed overnight in 10% neutral buffered formalin, briefly washed with PBS and transferred into 70% ethanol, processed and embedded into paraffin. Sections were cut at 7 μm for Haematoxylin & Eosin staining.

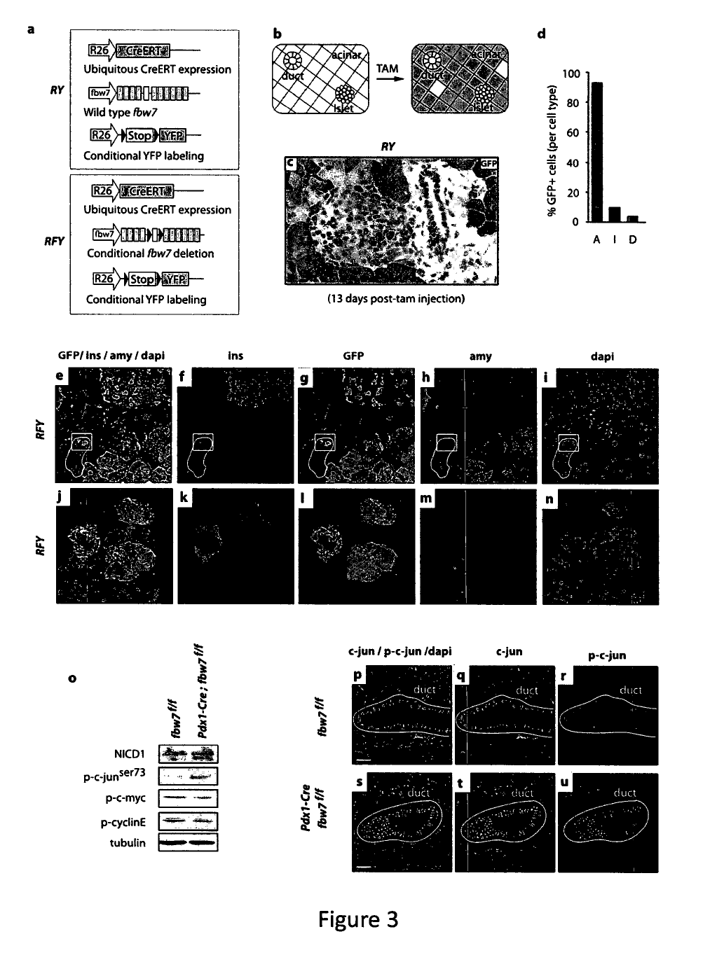

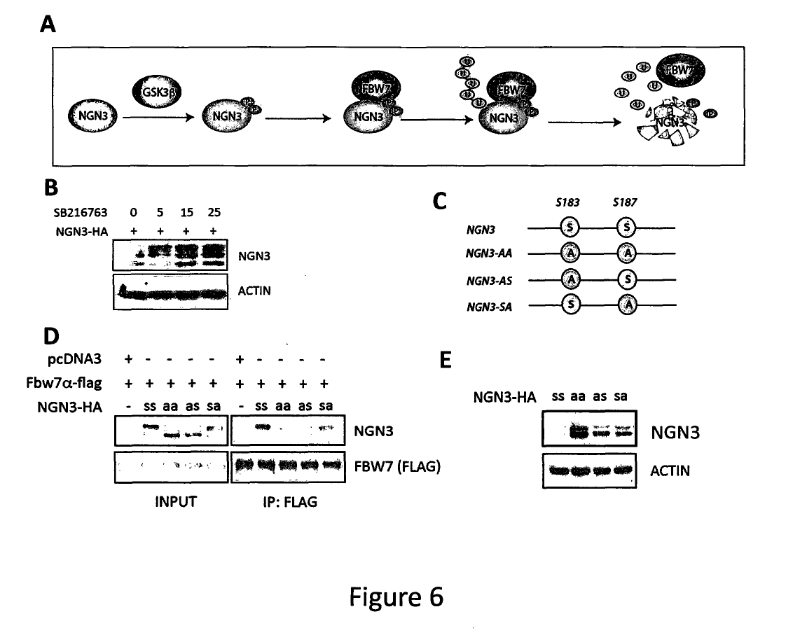

Immunofluorescence and immunohistochemistry staining was performed as described before¹³ using antibodies against GFP (Roche Applied Science), glut2 (Millipore), insulin (Sigma), amylase (Santa Cruz) and glucagon (Sigma) were used. The percentage of GFP positive cells per cell type (see FIG. 3 and FIG. 6) was determined in at least 5 sections per mouse (number of mice>3 per genotype).

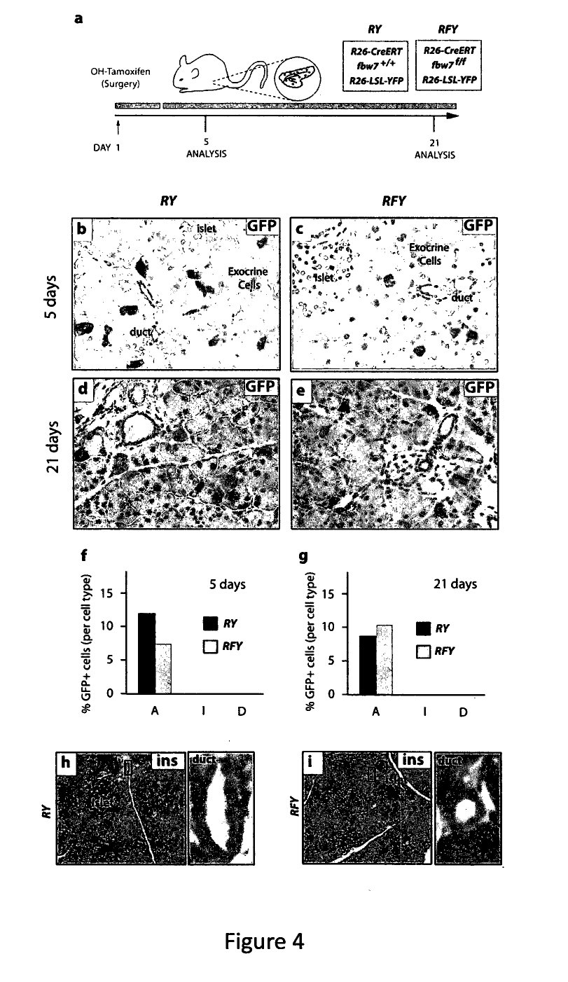

Quantification of number of ducts per area, number of isolated insulin clusters per area, number of insulin clusters in close proximity to a duct per area and number of insulin clusters within a duct (see FIG. 4) was performed on at least 10 sections per mouse (3 mouse per genotype).

### Electron Microscopy

EM analysis was done by the Electron Microscopy service at London Research Institute (Cancer Research UK). Briefly, dissected pancreas was fixed in 4% paraformaldehyde and 0.1% glutaraldehyde for 2 h at room temperature (24° C.). For conventional transmission electron microscopy, samples were further fixed by osmium tetroxide, embedded in Epon resin and sectioned at 60-80 nm. Images were obtained with a Tecnai G² Spirit BioTWIN transmission electron microscope.

### Western Blot Analysis

Pancreata were dissected out and lysates were homogenized in RIPA lysis buffer supplemented with protease inhibitor (Sigma). Immunoblots were carried out as previously described²⁵. The gels were transferred to nitrocellulose membranes, and the membranes were immunoblotted with various antibodies as indicated: NICD-1 (Abcam), p-c-Junser73 (Santa Cruz), p-Myc (Santa Cruz), p-cyclin E (Santa Cruz) and actin (Sigma).

### Statistics

Statistical evaluation was performed by Student's unpaired t test. Data are presented as mean±SEM. **p≦0.001 was considered highly statistically significant; ***p≦0.0001 was considered very highly statistically significant.

### Cell Culture and Transfection

HEK293T cells were cultured in DMEM and 10% FBS. Cells were plated at subconfluence and transfected with Lipofectamine Reagent (Invitrogen) following the manufacturer instructions.

### Sphere Cultures

Pancreas isolated from the mice with the indicated genotypes was digested in collagenase (1.6 mg/ml—Worthington Biochemical Corporation) at 37° C. during 30 min at constant shaking. Pancreas tissue homogenate after digestion was passed through a Nylon mesh (70 μm). Eluted cells were cultured under self-renewal conditions in Neurobasal Medium (Invitrogen) supplemented with 1% Penicillin/Streptomycin (10000 U/ml; Invitrogen), 1% L-glutamine (200 mM; Invitrogen), 2% B27 supplement (Invitrogen), human epidermal growth factor (EGF; 20 ng/ml; PeproTech) and fibroblast growth factor (FGF-basic; 20 ng/ml; PeproTech). Cells were plated in non-adherent or adherent 24-well plates as indicated. For differentiation, the primary spheres were mechanically dissociated and replated in 24 well plates.

### Plasmids

PCR generated NGN3-HA was cloned into pDEST-Flag using Gateway® technology. All the phosphomutants forms were generated by PCR-direct mutagenesis and cloned in pDEST-Flag using Gateway® technology. For the generation of the construct to generate the NGN3-AA transgenic mouse, the PCR product (NGN3-AA) was cloned into pRosa26-DV1 plasmid using Gateway® technology.

### Immunoprecipitation

Cells transfected with the indicated plasmids were treated with proteasome inhibitor for 3 hours. Cells were then lysed for 30 min at 4° C. in 500 μl of immunoprecipitation (IP) buffer containing 0.5% Triton X-100, 1 mM EDTA, 100 μM Na-orthovanadate, 0.25 mM PMSF, and protease inhibitor mixture (Sigma) in PBS. After centrifugation, supernatants were incubated for 3 h at 4° C. with 1 μg of the Flag or HA antibody (Sigma) coupled to Protein A-Sepharose beads. IP products and whole lysates were run in a 12% gel, transferred to a nitrocellulose membrane and immunoblotted with anti-HA or anti-Flag antibodies.

### Western Blot Analysis

HEK293T cells lysates or tissues were homogenized in RIPA lysis buffer supplemented with protease inhibitor (Sigma). Immunoblots were carried out as previously described⁴. The gels were transferred to nitrocellulose membranes, and the membranes were immunoblotted with various antibodies as indicated: anti-NGN3, anti-flag, anti-HA, and anti-Myc.

### Quantitative RT-PCR

For qRT-PCRs, cDNA was synthesised using Invitrogen Superscript reagents according to the manufacturer's instructions. Quantitative real-time PCR was accomplished with SYBRGreen incorporation (Platinum® Quantitative PCR SuperMix-UDG w/ROX, Invitrogen) using an ABI7900HT (Applied Bioscience), and the data were analyzed using the SDS 2.3 software. Results were normalized to those obtained with β-actin and results are presented as fold induction over control mice.

The sequences of the primers used for Q-PCR analysis were:

| | |
|---|---|
| mHes1-Fw: | 5′-TGCCAGCTGATATAATGGAGAA-3′ |
| mHes1-Rv: | 5′-CCATGATAGGCTTTGATGACTTT-3′ |
| mIns1-Fw: | 5′-CAGAGAGGAGGTACTTTGGACTATAAA-3′ |
| mIns1-Rv: | 5′-GCCATGTTGAAACAATGACCT-3′ |
| mIns2-Fw: | 5′-GAAGTGGAGGACCCACAAGT-3′ |
| mIns2-Rv: | 5′-AGTGCCAAGGTCTGAAGGTC-3′ |
| mPdx1-Fw: | 5′-GAAATCCACCAAAGCTCACG-3′ |
| mPdx1-Rv: | 5′-CGGGTTCCGCTGTGTAAG-3′ |
| mNeuroD1-Fw: | 5′-CCTGCGCTCAGGCAAAA-3′ |
| mNeuroD1-Rv: | 5′-GCTGGGACAAACCTTTGCA-3′ |
| mNGN3-B-Fw: | 5′-GCGCCTCATCCCTTGGA-3′ |
| mNGN3-B-Rv: | 5′-CCGGGAAAAGGTTGTTGTGT-3′ |

### Immunostaining

Mice were euthanised by cervical dislocation and the pancreas was dissected out. The pancreas was fixed overnight in 10% neutral buffered formalin, briefly washed with PBS, transferred into 70% ethanol, processed and embedded into paraffin. Sections were cut at 4 μm for immunohistochemistry. For immunohistochemistry, an antibody against insulin was used.

### Immunofluorescence

Sphere derived differentiated cells were washed twice with cold PBS and fixed with 4% PFA at room temperature for 15 minutes. Cells were washed with PBS and permeabilized with PBS+0.1% Triton for 10 min. Cells were washed with PBS and blocked in 1% BSA/PBS before addition of primary antibodies diluted 1:1000 in blocking buffer. Cells were washed three times for 5 minutes with blocking buffer followed by the addition of secondary antibodies diluted 1:400 in blocking buffer for 1 hour. Cells were washed with PBS and incubated with DAPI prior to visualising on a Zeiss Axioplan 2 upright microscope using AxioVision 4.1 imaging software.

### Transgenic Mouse Generation

Transgenic mouse generation was performed by the transgenic unit at the Cancer Research UK London Research Institute.

### Results

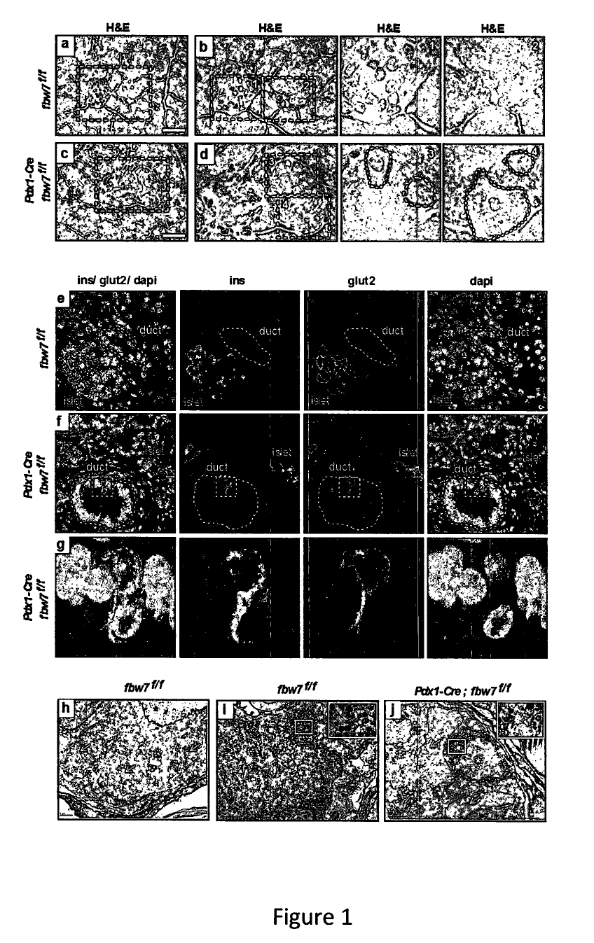

To assess the role of fbw7 during pancreatic cell-type specification, mice in which fbw7 was deleted in pdx1-positive pancreatic progenitor cells were analysed during embryonic development by generating Pdx1-Cre; fbw7^{f/f} mice. While pancreatic organ size and gross morphology appeared normal, histological analysis revealed some scattered cells in the Pdx1-Cre; fbw7^{f/f} ducts with altered morphology when compared with fbw7^{f/f} ducts (see FIG. 1a-d). These mutant cells were characterized by enlarged cytoplasm and a nuclear shape different from surrounding ductal cells (see FIG. 1 b-1,2 and FIG. 1 d-3-4). Insulin expression, which is normally restricted to islets of Langerhans in control mice (see FIG. 1e), was detected in mutant ductal cells (see FIG. 1f,g). In addition to insulin-positive cells, Pdx1-Cre; fbw7^{f/f} ducts also contained, albeit more infrequently, cells expressing the β-cell marker glucagon, while insulin-, glucagon- or amylase-positive cells were never found in control fbw7^{f/f} pancreatic ducts. Quantification showed that insulin-positive cells were most abundant in the Pdx1-Cre; fbw7^{f/f} ducts (16.82% of the ducts contained insulin-positive cells compared to 2.84% and 1.89% for glucagon- and amylase-positivity, respectively. Thus, absence of fbw7 appears to trigger abnormal differentiation of a small subset of ductal cells, with a preference for insulin-positive cells.

One of the key features of functional β-cells is the expression of the glucose transporter 2 (glut2). In control animals, glut2 was co-expressed with insulin in islet β-cells (see FIG. 1e). Double insulin/glut2 immunofluorescence (IF) demonstrated that glut2 is expressed in insulin-positive mutant ductal cells (see FIG. 1f-g). To characterise further the induced β-cells found in Pdx1-Cre; fbw7^{f/f} mice, these cells were analysed by electron microscopy. At the ultrastructural level, control ductal cells showed no accumulation of secretory granules (see FIG. 1h), whereas islet β-cells possessed small dense secretory granules characteristic of insulin granules (see FIG. 1i). In mutant ducts, cells that resembled islet β-cells at the ultrastructural level were found surrounded by ductal cells (see FIG. 1j). Thus, induced insulin-positive cells in Pdx1-Cre; fbw7^{f/f} ducts showed functional hallmarks of mature β-cells.

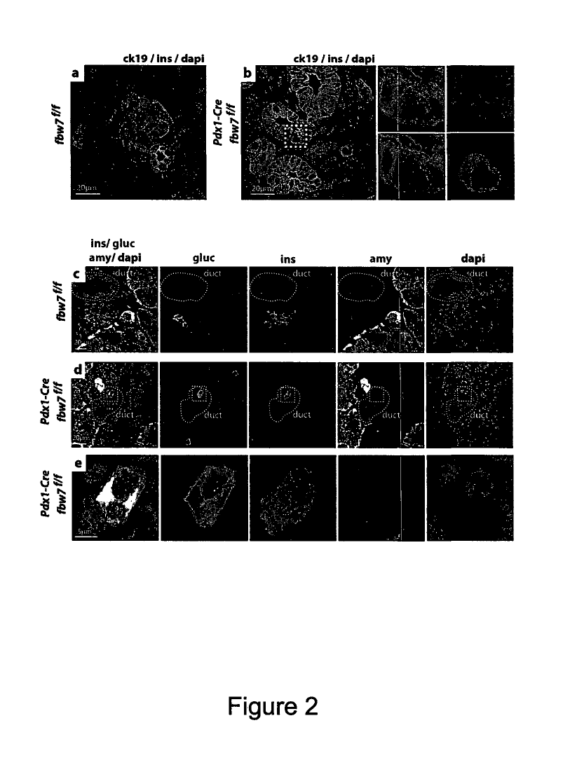

To analyse the induced β-cells, double immunofluorescence for markers of pancreatic cell types was performed. In fbw7^{f/f} control adult pancreas, cytokeratin-19 (ck19), a bona fide marker of ductal cells, and insulin were never co-expressed (see FIG. 2a), but co-expression of insulin and ck19 was commonly observed in Pdx1-Cre; fbw7^{f/f} ducts (see FIG. 2b). Triple amylase-insulin-glucagon IF revealed the existence of glucagon/insulin double positive cells in Pdx1-Cre; fbw7^{f/f} ducts but no amylase/insulin co-staining was found (see FIG. 2c-e). It is noteworthy that glucagon/insulin double positive endocrine progenitor cells have also been described in human embryonic pancreas¹¹. Thus, deletion of fbw7 in the pancreas promotes the occurrence of cells co-expressing markers of different pancreatic cell lineages, a cellular phenotype which is not normally observed in adult pancreas.

During embryonic development, Pdx1-expressing progenitors give rise to all the pancreatic lineages¹². Thus, the emergence of insulin-positive cells found in Pdx1-Cre; fbw7^{f/f} ducts may reflect a developmental-specific defect. To test whether fbw7 deletion was sufficient to induce β-cell neogenesis in adulthood, and to clarify the cells of origin giving rise to the induced β-cells, fbw7 inactivation was combined with lineage tracing. To this end, the R26-CreERT line, which expresses a tamoxifen-inducible form of cre recombinase from the ubiquitous ROSA26 (R26) promoter, was employed in combination with R26-LSL-YFP, in which Cre-mediated recombination leads to the permanent expression of YFP (see FIG. 3a-c). In an fbw7^{f/f} background, tamoxifen treatment will result in recombination of the floxed fbw7 alleles concomitantly with activation of YFP expression in Cre-expressing cells, and thus recombined cells will be irreversibly labeled with YFP expression driven by ubiquitous R26 promoter (see FIG. 3b).

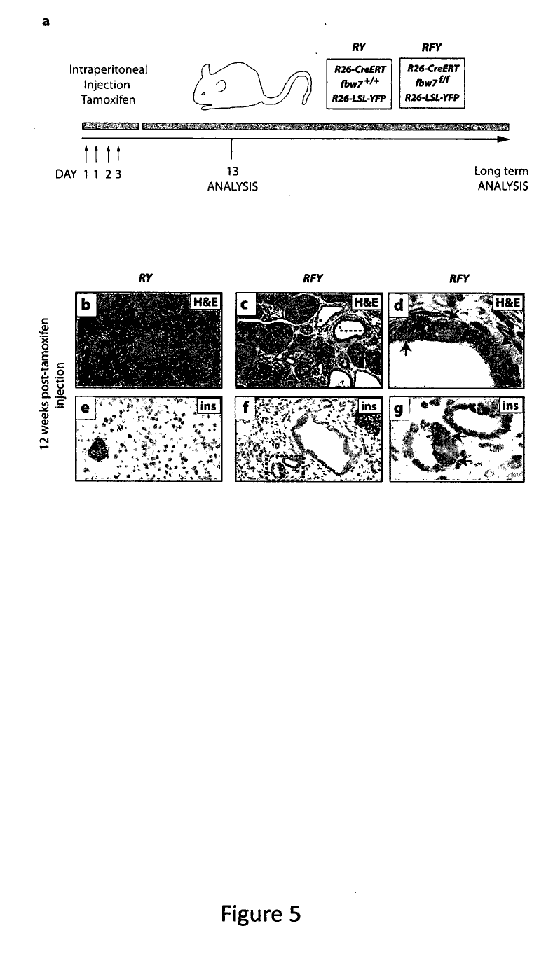

Adult (12 week old) R26-CreERT; R26-LSL-YFP (referred to as RY hereafter) and R26-CreERT; fbw7^{f/f}; R26-LSL-YFP (referred to as RFY hereafter) mice were injected intraperitoneally with tamoxifen and histological analysis of the pancreas was performed 2 and 12 weeks post injection. CreERT mediated recombination was detected in all the pancreatic cell types, but with different efficiencies. While almost all acinar cells showed YFP positivity (97%), recombination occurred much less frequently in islet (12%) and ductal cells (5%) (see FIG. 3c-d). Despite the high percentage of recombination in the acinar compartment (see FIG. 3d), no insulin-positivity was observed in cells with acinar cell morphology in RFY pancreas (see FIG. 3e-n). Likewise, direct intrapancreatic injection of 4-OH-Tamoxifen into the pancreatic tail resulted in exclusive recombination in acinar cells (see FIG. 4a-e), with no recombination detectable in islets or in the ducts (see FIG. 4f-g), but no β-cell neogenesis was observed (see FIG. 4h-i). In contrast, despite the low percentage of recombination (5%), ductal cells of RFY mice gave rise to insulin/GFP double-positive cells in ducts as early as 13 days post injection, albeit very infrequently (in less 1% of ducts) (see FIG. 3e-n). Insulin-positive cells in the ducts of RFY mice could also be observed at later time points after tamoxifen injection (12 weeks) (see FIG. 5b-g). Therefore, fbw7 deletion in ductal cells but not in acinar cells, induces the acquisition of β-cell identity.

Fbw7 targets many proteins involved in proliferation and differentiation such as N-terminally phosphorylated c-Jun, Notch intracellular domain 1 (NICD1), phosphorylated c-Myc and phosphorylated CyclinE for proteasomal degradation³. It has been shown that SCF(Fbw7)-mediated degradation of its substrates is tissue specific^{4,13-16}. Pdx1-Cre; fbw7^{f/f} pancreas showed increased p-Jun^{ser73} and NICD1 protein levels when compared to fbw7^{f/f} as detected by western blot while—Myc and CyclinE protein levels were unaffected (see FIG. 3o). In control pancreas, c-Jun and p-c-Jun^{ser73} were mainly expressed in the ductal compartment (see FIG. 3p-u), and c-Jun and p-c-Jun^{ser73} immunoreactivity was increased in Pdx1-Cre; fbw7f/f pancreas (see FIG. 3s-u), suggesting that SCF (Fbw7) controls substrate abundance in pancreatic ductal cells.

Ectopic expression of combinations of transcription factors can induce changes of cellular fates (reviewed in ¹⁹), however examples of reprogramming in vivo by loss of only a single molecule are rare. pax5 inactivation converts differentiated B cell into other hematopoietic cell lineages²⁰. In the present study, it has been shown that fbw7 controls the differentiation of a subset of ductal cells into β-cells and other pancreatic lineages, implying an unexpected plasticity and heterogeneity of pancreatic ductal cells, which might be therapeutically exploitable.

The conversion of pancreatic ductal cells into β-cells observed in the RFY mice in vivo could be due to up-regulation of substrates involved in beta cell neogenesis in the adult pancreas. One of such factors is NGN3, which has been shown to be required for proper differentiation of beta-cells from pancreatic progenitors⁶. To test whether Fbw7 had any effect on NGN3 protein stability (see FIG. 6a), a set of in vitro experiments were conducted. In accordance to the behaviour of most of the known Fbw7 substrates, NGN3 protein stability was increased when cells were treated with GSK3β inhibitor SB216763 (see FIG. 6b). An in silico search for putative phosphorylation motifs revealed the existence of a GSK3β consensus site comprising 5183 and 5187 (see FIG. 6c). NGN3 was able to interact in vitro with Fbw7 when both proteins were coexpressed. However, the interaction was abolished when Ser183 in NGN3 was mutated to Ala (see FIG. 6d). Furthermore, the stability of NGN3 when mutated in the GSK3β phosphorylation site was drastically increased (see FIG. 6e). These data lead us propose the following mechanism of action: Fbw7 expressed in a subset of pancreatic ductal progenitors would be controlling the levels of NGN3 (see FIG. 7a-8a). In the absence of Fbw7, NGN3 would accumulate in those cells, thus initiating the differentiation of them into β-cells (see FIG. 7b). This model predicts that overexpression of NGN3-AA (in which the GSK3(3 phosphorylatable serines are mutated to alanine) in mouse pancreas should increase beta cell number (see FIG. 8b).

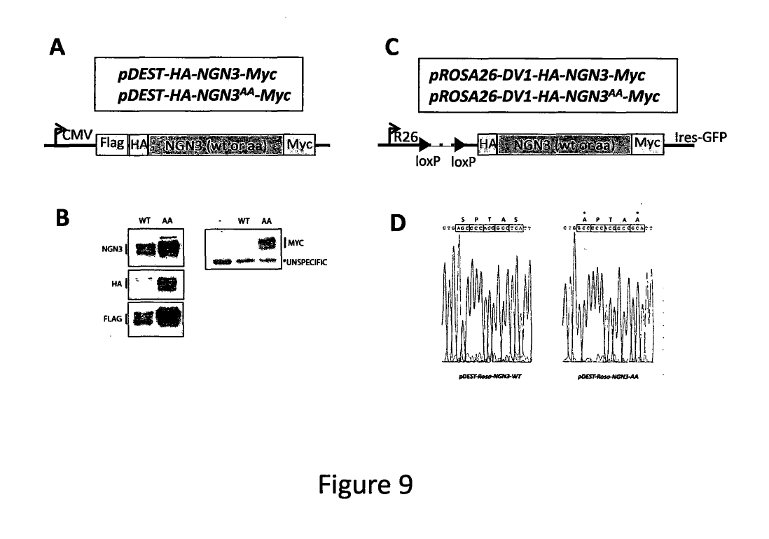

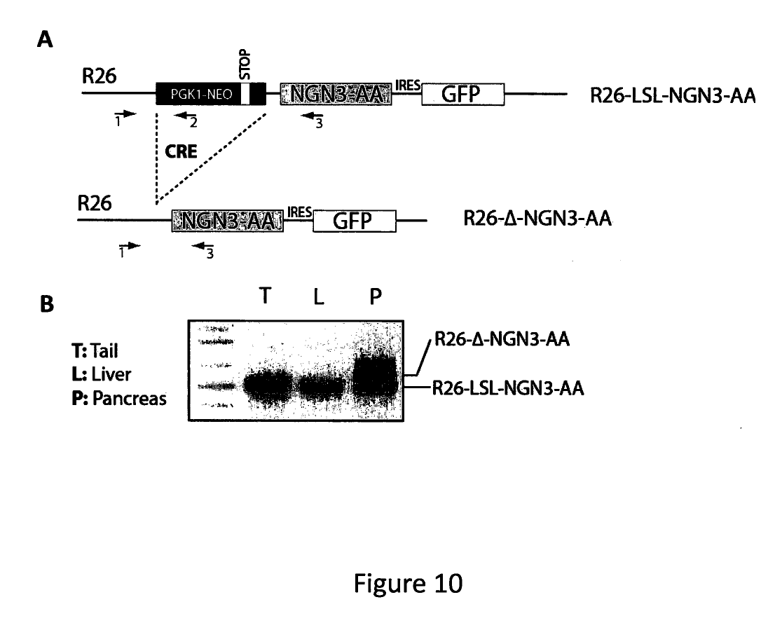

To test this possibility, a mouse line that would allow conditional overexpression NGN3-AA in the pancreas was generated (see FIG. 9a-d). In order to achieve overexpression of NGN3-AA specifically in the mouse pancreas, we crossed NGN3-AA mice with Pdx-1-Cre mice (see FIG. 10a). NGN3-AA; Pdx1-Cre mice showed good recombination of the R26-LSL-NGN3-AA allele in the pancreatic tissue, while liver or tail remained completely unrecombined (see FIG. 10b).

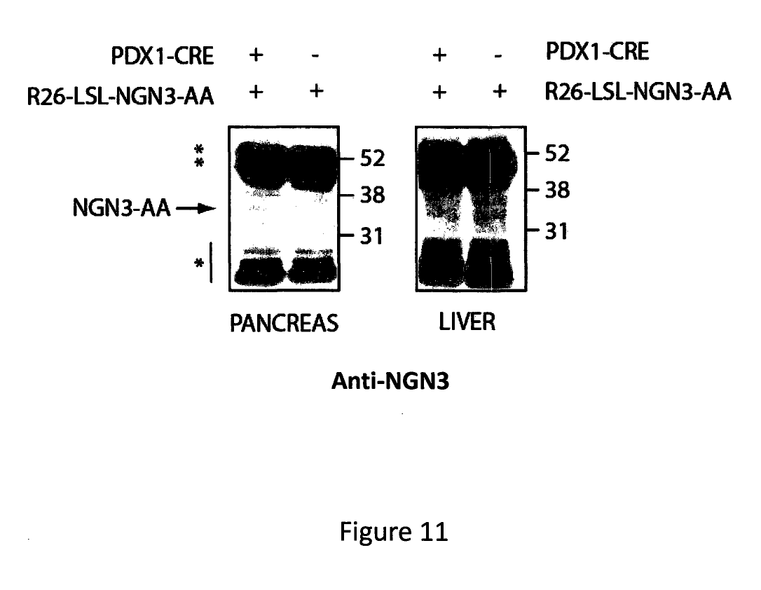

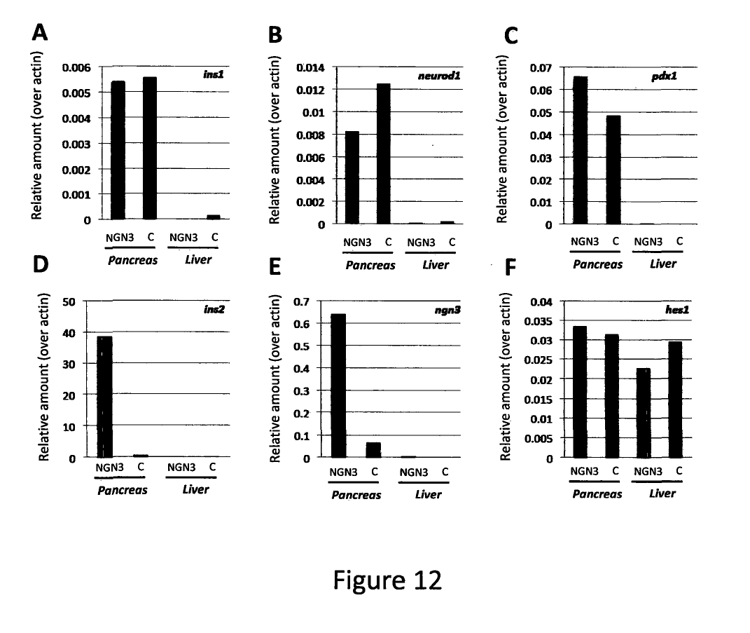

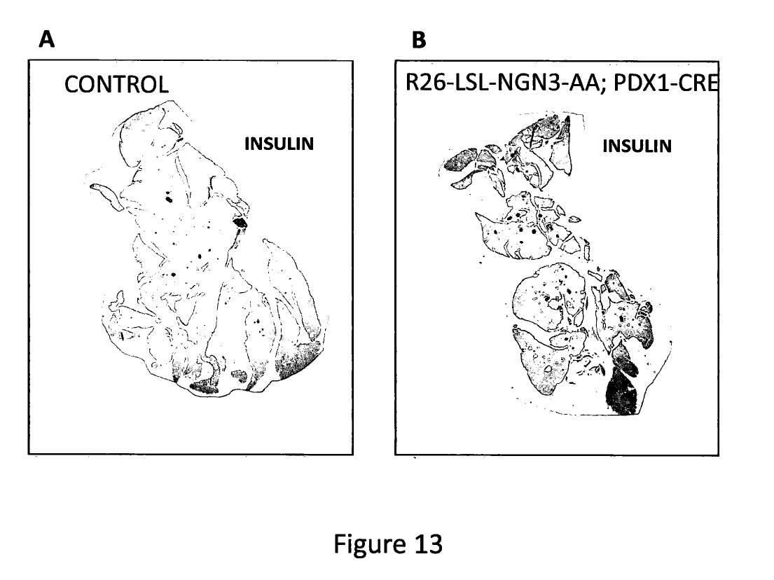

Accordingly, NGN3-AA protein was detected in the pancreas but not in the liver (see FIG. 11) concomitant with an increase in the amount of NGN3 and Ins1 mRNA (see FIG. 12). Furthermore, the number of insulin islets analysed by immunohistochemistry performed in NGN3-AA; Pdx1-Cre pancreas was increased when compared to control littermates (see FIG. 13). Thus, over-expressing NGN3-AA in mouse pancreas is sufficient to cause an increase in β-cells. Therefore, NGN3 is a likely substrate through which Fbw7 exerts its function. These data suggest that, inhibition of NGN3 degradation by Fbw7, for example by inhibition of GSK3-β, may cause the de-novo generation of β-cells.

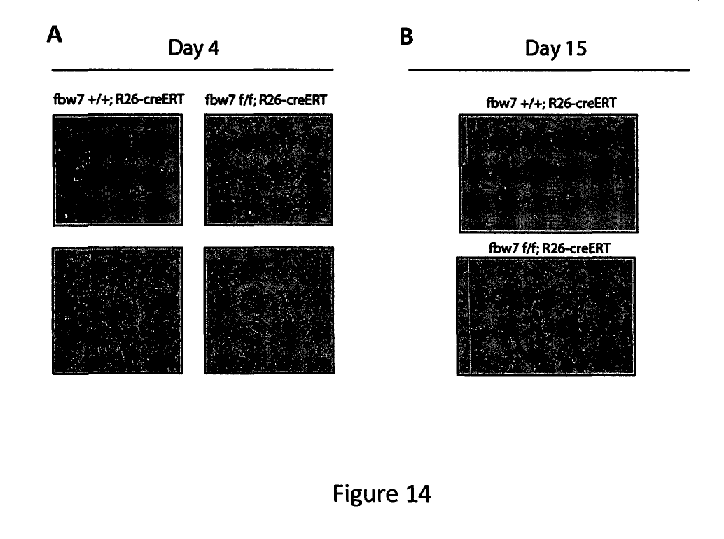

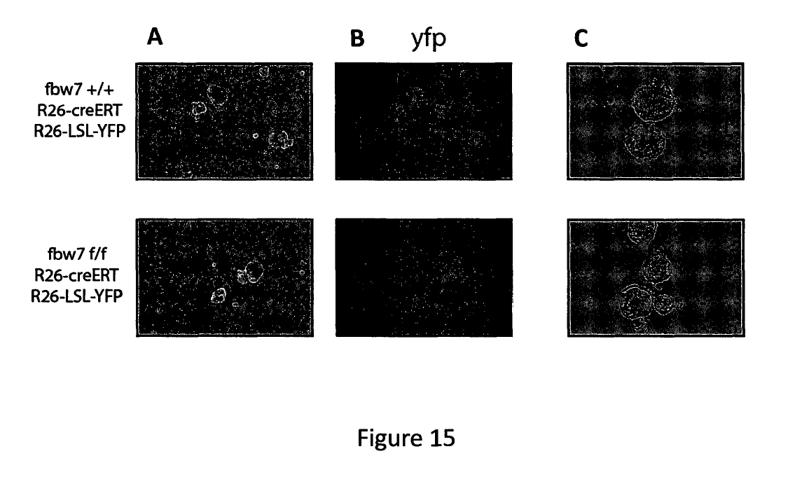

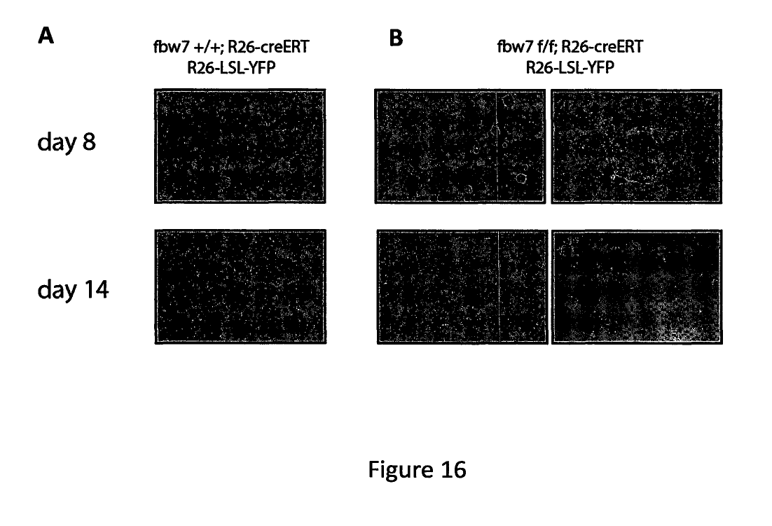

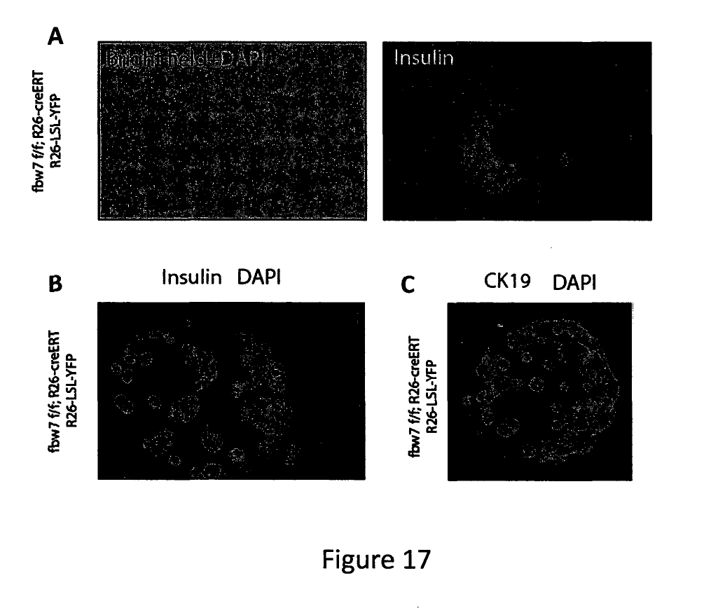

To address the differentiation potential of pancreatic spheres in RFY and RY mice injected with tamoxifen, an in vitro culture system that enriches for ductal cells was optimized. Cells isolated and cultured as described in the Methods section were monitored over time. As early as day 4 post-plating, pancreatic spheres were observed in the cultures and remained constant until 15 days post-plating (see FIG. 14). Both RFY and RY spheres showed GFP positivity under the fluorescence microscope, suggesting they were originated from recombined pancreatic cells (see FIG. 15). Furthermore, when day 4 spheres were dissociated and replated in attachment culture conditions, some RFY spheres spontaneously differentiated into adherent cells after day 8 and the effect was even more drastic after 14 days post-replating (see FIG. 16). Interestingly, Insulin and Ck19 staining revealed that both cell types coexisted in the differentiated structures (see FIG. 17). These data suggest that Fbw7 deletion spontaneously induces the differentiation of pancreatic spheres into β-cells in vitro.

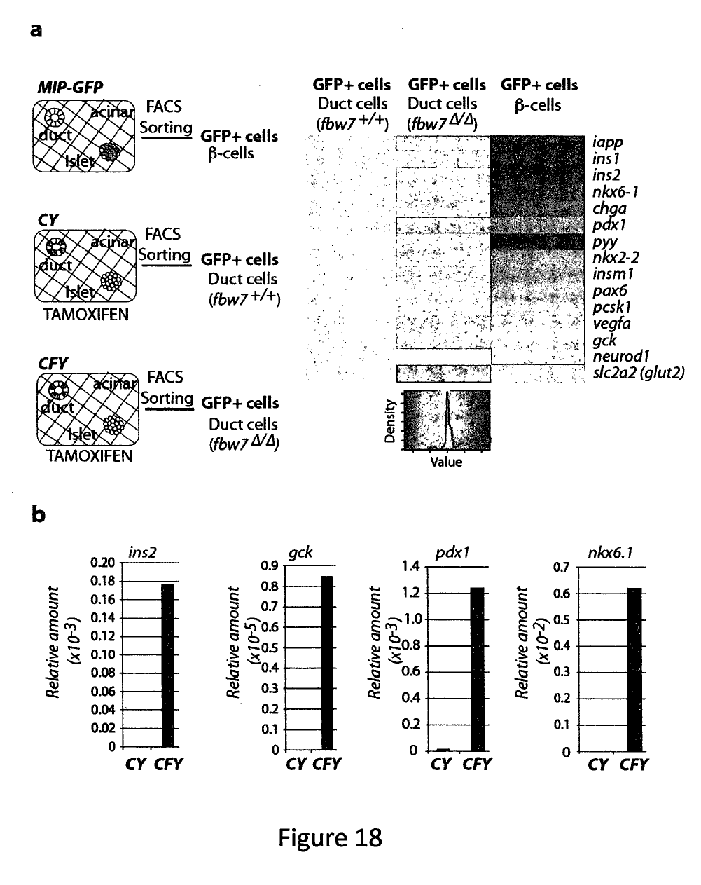

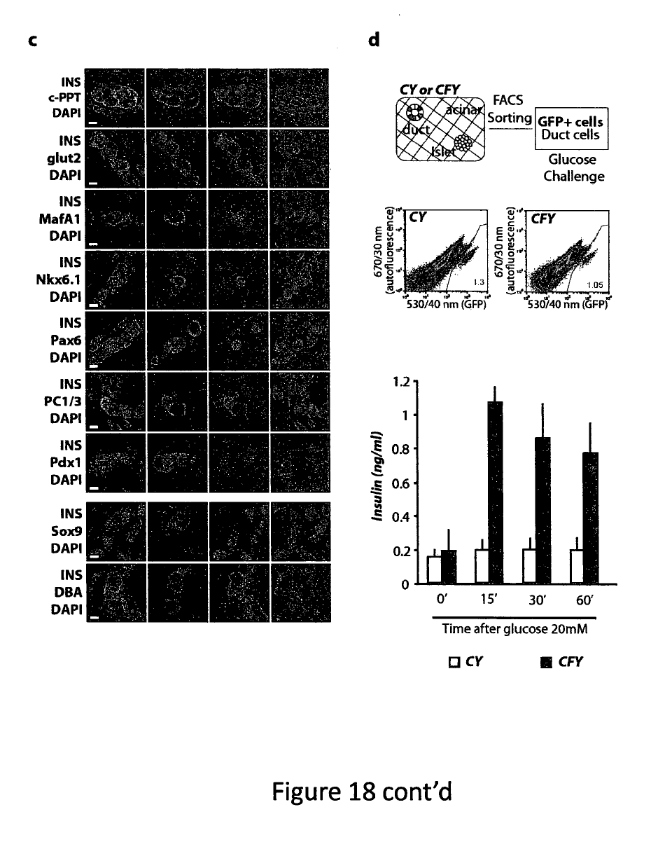

To explore further the functionality of the newly formed β-cells upon fbw7 deletion, mRNA expression profiling of GFP+ sorted cells from tamoxifen injected CY and CFY mice was carried out and the results were compared to those from GFP+ cells from MIP-GFP mice as a positive control for β-cells (FIG. 18A). CFY GFP+ ductal cells showed increased expression of β-cell specific genes iapp, ins2, nkx6-1, chga, pdx1, pyy, nkx2-2, insm1, pax6, pcsk1, vegfa, gck and slc2a2 (FIG. 18-A). Q-PCR analysis confirmed increased mRNA expression of β-cell marker genes (ins2, gck, pdx1 and nkx6.1) in CFY ductal cells when compared to CY GFP+ cells (FIG. 18-B). Furthermore, insulin+ cells in the ducts of CFY mice co-stained with the functional β-cell markers c-PPT, Glut2, MafA1, Nkx6.1, Pax6, PC1/3 and Pdx1 (FIG. 18-C) while they were negative for the ductal markers Sox9 and DBA (FIG. 18-C).

An important hallmark of β-cell function is the ability to release insulin upon glucose stimulation. To test this in induced β-cells, GFP+ cells from CY and CFY mouse pancreas were sorted and challenged with glucose treatment in vitro (FIG. 18-D). While GFP+ cells from CY pancreas did not respond to glucose, GFP+ cells from CFY mice showed a substantial release of insulin (FIG. 18-D). Thus the induced β-cells show both the characteristic marker expression and functionality of mature β-cells.

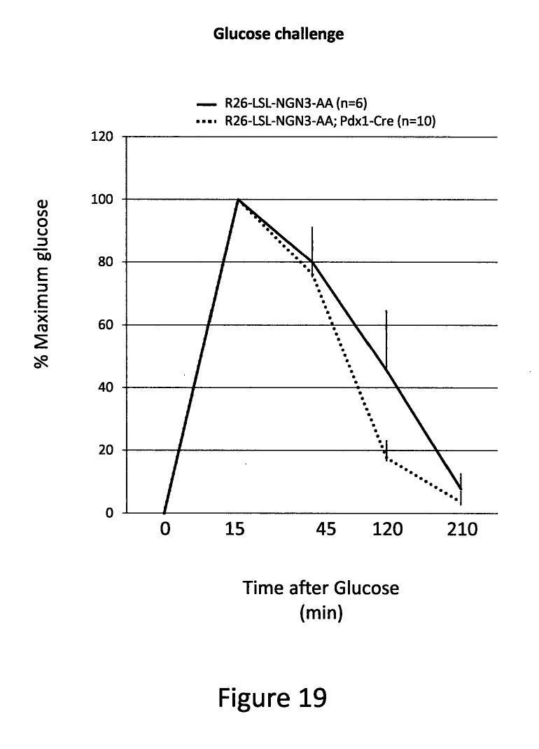

A glucose tolerance test was performed in R26-LSL-NGN3-AA and R26-LSL-NGN3-AA; Pdx1-Cre mice. Mice were fasted for 12 h before glucose injection. Glucose levels in the blood were measured at the indicated times. The data suggest that NGN3-AA transgenic mice show improved glucose tolerance compared to control mice (FIG. 19).

TABLE 1
Fbw7 protein sequence

| | | |
|---|---|---|
| Fbw7 protein | Human (Homo | MNQELLSVGSKRRRTGGSLRGNPSSSQVDEEQMNRVVEEE |
| sequence | sapiens) | QQQQLRQQEEEHTARNGEVVGVEPRPGGQNDSQQGQLEEN |
| | NCBI Reference | NNRFISVDEDSSGNQEEQEEDEEHAGEQDEEDEEEEEMDQ |
| | Sequence: | ESDDFDQSDDSSREDEHTHTNSVTNSSSIVDLPVHQLSSP |
| | NP_361014.1 | FYTKTTKMKRKLDHGSEVRSFSLGKKPCKVSEYTSTTGLV |
| | | PCSATPTTFGDLRAANGQGQQRRRITSVQPPTGLQEWLKM |
| | | FQSWSGPEKLLALDELIDSCEPTQVKHMMQVIEPQFQRDF |
| | | ISLLPKELALYVLSFLEPKDLLQAAQTCRYWRILAEDNLL |
| | | WREKCKEEGIDEPLHIKRRKVIKPGFIHSPWKSAYIRQHR |
| | | IDTNWRRGELKSPKVLKGHDDHVITCLQFCGNRIVSGSDD |
| | | NTLKVWSAVTGKCLRTLVGHTGGVWSSQMRDNIIISGSTD |
| | | RTLKVWNAETGECIHTLYGHTSTVRCMHLHEKRVVSGSRD |
| | | ATLRVWDIETGQCLHVLMGHVAAVRCVQYDGRRVVSGAYD |
| | | FMVKVWDPETETCLHTLQGHTNRVYSLQFDGIHVVSGSLD |
| | | TSIRVWDVETGNCIHTLTGHQSLTSGMELKDNILVSGNAD |
| | | STVKIWDIKTGQCLQTLQGPNKHQSAVTCLQFNKNFVITS |
| | | SDDGTVKLWDLKTGEFIRNLVTLESGGSGGVVWRIRASNT |
| | | KLVCAVGSRNGTEETKLLVLDFDVDMK |
| | | (SEQ ID NO: 1) |
| | Mouse (Mus | MNQELLSVGSKRRRTGGSLRGNASSSQVDEGQMNRVVEED |
| | musculus) | PQQQARHQEEEHTARNGELVGANPRPGDQNDTQQGQVEEN |
| | NCBI Reference | NNRFISVDEDSSGNQEEQEEDEEHAGEQEEEEEEEEEEEE |
| | Sequence: | MDQESDDFDPSDDSSREDEHTHNSNVTNCSSVSDLPAHQL |
| | NP_001171245.1 | SSPFYTKTTKMKRKLDHGSEVRSFSLGKKPCKVSDYTSTT |
| | | GLVPCSATPTTFGDLRAANGQGQQRRRITSVQPPTGLQEW |
| | | LKMFQSWSGPEKLLALDELIDSCEPTQVKHMMQVIEPQFQ |
| | | RDFISLLPKELALYVLSFLEPKDLLQAAQTCRYWRILAED |
| | | NLLWREKCKEEGIDEPLHIKRRKIIKPGFIHSPWKSAYIR |
| | | QHRIDTNWRRGELKSPKVLKGHDDHVITCLQFCGNRIVSG |
| | | SDDNTLKVWSAVTGKCLRTLVGHTGGVWSSQMRDNIIISG |
| | | STDRTLKVWNAETGECIHTLYGHTSTVRCMHLHEKRVVSG |
| | | SRDATLRVWDIETGQCLHVLMGHVAAVRCVQYDGRRVVSG |
| | | AYDFMVKVWDPETETCLHTLQGHTNRVYSLQFDGIHVVSG |
| | | SLDTSIRVWDVETGNCIHTLTGHQSLTSGMELKDNILVSG |
| | | NADSTVKIWDIKTGQCLQTLQGPSKHQSAVTCLQFNKNFV |
| | | ITSSDDGTVKLWDLKTGEFIRNLVTLESGGSGGVVWRIRA |
| | | SNTKLVCAVGSRNGTEETKLLVLDFDVDMK |
| | | (SEQ ID NO: 2) |

TABLE 2a
Human Fbw7 nucleotide sequence
Human (homo sapiens) Fbw7 nucleotide sequence
NCBI Reference Sequence: NC_000004.11 (SEQ ID NO: 3)

ccttccgcagctgccgcttcagtccgaaggaggaagggaaccaacccactttctcggcgccgcggctcttttcta
aaagtgtgagtggccccgggagagggaattgagggggagaaagtgggaggatgccggcgcttcctcctggagttt
tgctgatgttctccagtagtgttgggagtctatcaatgagggagtctagctttattcacggaggggggaaggtgt
tagggaggcctgtattttgtaactttttagctctttgtacgagtaatgcaagtatgcaggtttctgtcggaggag
tcttttctccaaagtgaatactcgcacctttctggccagcagttggcaaagggaaattgaggaagccagagcata
tgtgcgcacagaggccatctgtatctgtaaccttttcctggtactgtgggtgtgtgcggtgtactttttgtttcg
ggggtactctcatataggttcccgaatgagaactacttaacctgtggacccctgaaagtaaaattccttttttaa
aaaacaacaacaaacctgttttgagagaggttcatttcagtacctattggtacaggagttaaccttctcagttac
tgggtccaatttcatgtgtgtcaggaggacactgtaacagatgtcagttactcaggaacttgagttgttaggaat
cacaacttacttggggaagagcatttatcacattaatcattttataaaagttgtgattttttccctttcaccccc
ccctttttttttttagagtaacaaattacagcacttttttatttggtttgaggctttgcttttttgaattatctt
tatttctgtatcaatttaggatacatctttgatatttaagtcaggaatgattttttccttccattattttaaagc
tgaagtttataaaacctttgacttgtattttaggtatcttgtcagagtaccaaattaaaggtttatgaaagtaat
taatatttcatatacagaccttaagggttaaacatagagcagaattttatgtaagaagacatagacagtagtata
ctgagaattgtggtccgtctagacaactgtgggctcgttctgtctgaagccactgctgagagatactttgtggga
atgaatgtaagcctggggttatctctcttttcgaagttctccattcaggatcttgattagattaatttaaaactt
tctcaaacttaacaatgtttggtccttttttttaaagtaatgagttctgcaagtttattagcctgtgtattatgt
gacagttcatttacatattccatacctgtgtccctaaactttcaggacacagtcagagtttgtgtttcaagaatt
atcaggacaagcctcagttcatcttttcttaattttatggactttcattatttctaccatcagtttctccaactg
ttgtcttagaataatgtctttcatttgaccacttttccctcctctggaacttctcccactgaattgtttttcttt
agttgccttgttatgctttggtaaggaactttaaattctgtcctttaaatgtgtactgacatgaaaatactgaat
agtggatgtaatgttagaatcagagtcacgtattctgtttttcactaagatattgcaaagagaaatgtagtcatt
ttctaaaatactgtacccaaaatacagtagaagctgtattttgggaaggaggggaggacaaactaagaaacaaac
tatagtttgaatgtaggttgcactctgtcaaagttgcttactaactttaggacctacttttattgcttatggatt
ggttaaaaattacggtattttttttttttttgagacagagtcttgctctgtcacccaggctggagtgcaatggag
cgatcttggctcactgcaacctccgcctcccaggttcaagcgattctcatgcctcagcctcccaaatagctggga
ctacaggcatgtgctaccacgcctggctaattttttttgtatttttagtagagatggggtttcaccatgtgggtc
aggctggtctagaactcctgacctcaaatgatctgcccacctcggcctcccaaagtgctgggattacaggcgttg
agacaccgcgtcctgccaaaaaattatggtaatgttttattgcacgtttgggtagaaatgacaaagtttttgggg
tccctgcctttgagatgcttttacttgtaggtggtggagacaatcaaacacaaattgcacaaagctataggagag
gggggatagaccaatatttatctaagaccgcactggatgttagatattgtcaacttatagatgtgaatatatttt
tacatttttcactagggaaatgttgcttttggacgtgtacatatagaactaattgaagtattgaagccaaatctg
ggaaggtttcttttaggaagaaaagagcaagtggagacacaatatttaagacttttggaattgaaaggcataagg
gacagataaaattacgaagctggtttgaagggaatacagatagaggttgttgagtagtgttagggtcagttgtgt
ttgtgtagacacatgattacagttcaaagaagcaactgagaagagtgttcctgagagtaatttcagatgactggg
gagaaattacattttaggtaaaagctgtatgcctggattaggattttgtttttctagggaaccaacatttttatt
atatcgtggctatccagggctatacagaaggaggagtgaaaaagaagatgcttagtgtgttgaaagtggcttctt
acaaaaaagagtttagaaatgggacagtagactttgctttggatgtgaaattttagggcaacaattggatactgg
tgcagtttcaaaacagcaaaatagtttagcaggtaatatactgaaaatgtaaagttggaattattttcaaagtat
ttaggtttcccagagtactgatggaaagtgaagagctgaaaagtgggtatttttagttagggggaaaatctgaat
gaatgagacactacctaaaatagatgttttagaagtggataagagttgaagttaaaaaggaatgagcaggcttta
aacatggccaccagtttagtcccagggtatattattctgtatttttgaaacaagctaaaattttttttttttttt
aaatagggtatataggtcccttgagattaagttcagaagagtatacttaataataatttcattgttaaggaattt
ataaatagaaggacagatttgtttcctcaaattcatgttttaaagttcactgtgcattattatcctacgtttggt
cttgatatgacgaagtgaggccaaaaatagctgacagagaaggctgcttcccttgttcacagttggtgtgtgtct
ttgtgtcccatacaatggaggggatatccctgtgtaggcacagtagtgctgaacatgctttttttttttttttcc
ttttaaactttttattaaaaaaaagcaacttggcatttaaaaaaatgtagacatttttgcggcttgcatttagta
ttgactttaacaggattaaggattgtgaggaaggaaatatatattcacacttttttgtgttccttagttggaatt
ctgcaccttcctagtagcttctctgacttcttaacagtgattattcaaatggcagaaactctggggaagagatgt
ttcaacaaggagataggtctaaggtgattctagggtgaaaatatagggattggattatatgccatatggaggcct
tctcttgtcatcctccactttccacaacattttttgtgcagtcgaagccatgttcagatgaattttagaaggcag
ccacttatagttaatgttatttactagtgaggacagtgggaagagtggtaacttcatgtactttgccttggcaac
ttttggattgatagggaattagagagccttgagggtatatgtttgtgtgtgtgtgtgtgttttagcacccaccag
atgaccccaaaggccatgcataggctcatttcattgtactttggagatggttcttggagaagtcttctggtgctt
ctgacatctcattgtgtagagaaagagttggcaaactatttttgtaaagggccagttagtaaatattttaggttt
tacaggccacatgatctgtcacaactttcaattctgttcttatagtgtgaaagcagctgtagacaacaaggaagt
gaatgaatgttgccgtgctccaattggcccataggccgaagtttttggactcctggtatagacaactgccaaatc
caccaacaaacagtatgagctttattacactacaaagccaggactggctatttttggtggtctttgttttctaga
tgagatgtttttctctgagttcatttaaaatgattctgcataaagcacatactctactgtacaaaatacttttgc
cttcatggtgatgaaaactatggattgagggtcggggtgataccatctttaaagggatgcagctaaaaagtaggg
actacctgcatttcttatctaaagataaccacagttggcatttagtgatatactggctttgtatcatggccaaaa
gtatctgtaaagaagcttgtgggcatacttttgaaaccttctgaaaataaatgtaaagcatttacttcgtgtgta
aaattagaagtgtctatggttggtttgaataatatgagtcaaattataagagcattaaagtttgatactgctatc
tattaaagtcttaatctcttgcagtggctaattcagtgtgtagaattacacatcgcaatttttgttttgttaatt
tcaagtactagtgtatattaacaaatcacttaaatcatttaaatgtgtactttaaaaagctttgatgtatataca
tatatatacgtatatatatatatgtacacgtatatatatacacgtgtgtgtgtgtgtgtgtgtgtgtgtgtgtgt
attttttttttttttgtgacggggtctcattctgtcacccaggctggagtgctgtggtgtgatctcagctcactg
caacctccacctcctgggctcaagtcatcctcctacctcagcctctcgagtggctgggatcacaggcatgcgcca
ccatgcccgactaatttttttgtatttttgatagagatgggtaacaacttttggattgatagggaattagagagc
ctcgagggtatatgtgtgtgtgttttagcacccaccaggtaaccccaaaggccatgcatcatgttgcccaggctg
gtctcttaactcctgagctcaggtaatccatctacctcgacctccctcagtgctgggattgcaggcgtgagccac
cacacctggccagcattgctatatttttaagagtgaatctttctcacatgtgttgttcagaaagtgtaattcact
ttgtgcaattttacattacagttaatattaaaatatttgtttcatatgtcagcagataccaacattggatgatgg
tatatttagtttcataaatcctatttattttaggattcccttgtttatggcaggggtggagattactcttgcaat
aatgatcactttttagttagtttgaaacctgcttttagtttatatgtagtatttctacatattgttacttaaatt
gaactaacaatttactttttaatttttcttcttccttttaaaaaaagagtatataatctgacaatgttagtacat
cttttagaaggtcactataaagttgctttccctatttatttggaaaggattatttccttattattttaaaaaatc
ttttaatttttgtcttttcaagtaattttatcattgtcccaagcctaaggatgagtggcaatttaaaagacacaa
ggtgtgcatcttctatctgcaaatactccaaacagaaattattccagtttgttgatactttgagtggaccaggga
aaaatgtgtatgtttttagtgtaactgaattgtcattacaaaaagatgagtaaagttgctgttaaataatgaatt
cttgctgttaatttgtcctgttcttagactttcccccattaagtataatgggggaaaatgtacacacacacacac
acacacaaagtatgtatgtgtagatatacctagcgtattgtgctagatgttcatttctgcatgactgctctggaa
tttaggcaatattaaaagaccttacaaaatatttttaatatcacttaatatcctgttttatgtcttaatgtactt
ttatgtttttgctgctttctgatgatacctccttggtacttcctcatgatacctttcacagtatgtttaaatgga
atgtattttctcaaagtacgtgacattttctgaacactatgggatatatatcttctagtaattatatttttacat
gcaagtatctttaaaaatttcttcctattacaacttaagaattgtgaaacatttgagggagcatctgcagtttga
gcacttgatctggatattggaatgacaaacttaacatttaactaaatttttctgaggtgttcttttacaattttt
gagttaacacttgatttctgtaaagcagaaggcttataagaagaaaatatgagagccagtgttcgtcttgtatta
tcctgccatgcacgtagttgaaaatcagaaacaaacagaaatgcacaagagaccttattgcttgagtatcttctg
ctcttcccccatcagacctaagggacagctagtggatcagtcagatactgtgcagtttctgttgctatgctgtgt
cacctttgccctttctttctatctggaagatagctactggaaatctggctgtaagaaagagatagcatgtttttt
tcctctctctctctgtgtgtttctgtctgtctgtgtacctgtacctagtgtattgtgttagatgttcatttctgc
atgactgctctggaatttaggaaatattaaaagactacaaaatatttttaatattatttagccttttctctgaga
ggatcacatcctctggctttggcgttttcatctcacagcatcttaaacgaacccgtcaatgtttattatcagttc
caaatgtgaagtctcaaggcaaattgttggtttgtttttggaagaatgtatgtgtgtgtgtatatatatgtgtgt
gtgtgtgtgtgtgtgtgtgtgtgtgtgtgtatataatttttaaatttttaacaaaaattcctgatctagtcaatg
cttatgagagtgtgtgtggtttgtataaaattattttttttagagacagggttgcttgttgtcaccaggctggag
ggcagtggtgtgatccttgctcactgcagccgtgaccccctgggctcaagtgatcttcccaccttagcctcctga
gtagctgagtagctgggactataggtgtgtactgccacaccctgcaaatttaaaaaaaaaattctgtaaagacag
ggtctcactatattgcccaggctggttctcaaactccaggactcaagcagtcctcctgaaatggcctcccaaagt
gttgagattacaggctggggccacagtgcccggtcagagtgtgtgtattttttaacagccttttaggattgcatg
ctttttaggggggtgaatgaggggtaatatacctttgcatgacattctctaggtctgagaaggagttgcttcact
aaaagtgtaatttataattaccaaatggttgaaaacaggtctacctggagtcagcaacaggcagctagtttactt
ctcatttacgtaataaaatacattcataatatgcattcagtaattcataattcaataatttcttggcttcatggc
cttggacaagctatttattatccttttaattttggtggcatcacctgttgatggaggaaaacgtgtcctttacat
ggtggttgttaggattaaatgagataatataaagcatgcaaagctcttaatccagtgcctggcatatagcatttg
acccatcagtgttaatttattaagttaaagttgttttaaagtgtgttgtagatagaagatttaaatcaggcctta
tattaagcccaacatttaaattcaacacttgagtttgacaaataatactgaagtaattctgtgttttcagaaaca
tctaaaacttcattaacattttattaaaaactgaagaaatgacagaactgttgggcttttttttaatagaaaaga
aagactgatttgaacataactggaatttgaatttagtttcaaagatctcagtagtgcatggaccagaaaagaatc
cgtttttgaatagttagtgattatgatagtgactgtggtttaaattgcccatttagaaatagcaggatctaattc
agttatcagcctttttagttgcccatctctttaacccctggaaataaaaagctttaaaaaatcattaagaagttg
aacggaaattttatctgtgtcatttacatctcaaattaagatcgaaataattttgtttacatgattctattttaa
ctacttaattttagtttttgttttttcctgaaattattagtatgtgggttttaaaaattcctttttttgtgtgct
taatatttgttaaatacaagattggttttcagaaataccttttcttcgagtagggcataatcagtgttttattgg
ccaatacaataaataaatgcttcttgattgtttttgtatttggaaagaaattctatgttgagaacagttttggta
tgctttccttcattggctgtttattgaatagaactgtatatttaaggtatatgaaaaaaatctaagctagaaaaa
aagactgagatacattcttggaagaaaatacttgcaaactatgcatctgacaaaggtctattgtccaacgtctat
aagaacttaaatttacaagagaaaaacgaccccattaaaaagtggacaaaggacgtgaatagacacttctcagaa
gaagacttacatgtggccaacaagcaaagaaagctcaatattactcattagagaaacgcaaatcaaaaccacaat
gagataccatctcagactattcagagtggctactataaaaagtcaaaaaataacagatgctagtgaagttgtgga
gaaaagggaacacttatatactgttggtgggagtgtaaattagttcaaccattgtggaaatcagtatcgcgattc
cttaaagagctaacagcagaactaccattccacccagcaatcccattactaatgggtatatacccagaagaatat
aattcattctaccataaagacacatgcacacgcgtgttccttacagcactaatcacaatagcaaagatgtggaat
caatctaaatgcccatcagtgacagattggataaagaaaatgtggtacatacacaccatgggatactgtgcagcc
ataaaaaagaaggagatcatgtcttttgtgggagtatggatgaagctggaggctattatccgtagcacactaatg
caggaacagaaaaccaagtaccgcgtgttctcacttataagtgggagctaaatggtaagaacttatgaacaaaat
gaatagacactggggtctacttgagggggagggttggaggagggagaagagcagaaaagataactattgggtact
gggtttaatacctggttgttgaaataataagtacagcacaccctcgtgacatgtgtttacctatgaaacctttac
atgtaccccgaaacctaaaataaaagtttaaaaaatatattctttagcactgattatcttgctagctgtgacttc
tccttttaaggaaagtttgctttagattttggtgtaaatgtagaaattagatgggtttatctgctagtggctctc
tagttagatgattaatgaatattctagtttaaaaaatttcagcattttaaatacttttctttattggtaacgatt
tggattatgcttctagggatctttacatttcatagtttttcaaaaaagactttataatgagttcctggatgggaa
gaagagcatgttctctctagtttatttgatttcttcctgattattatgtatgtaatactggaaggttgtgtaaat
aaatatttgggttgtaagttttcttttgcatgattggtggtctcttaattgactgctgtgtgaatttgatagtat
aaactagaatgcttatacagtgagtggggaagcctttttctactgagtgttatgcaggtggatttttcccccctg
cggataaatactaaacttttatattaccagatttagacatttagacaggaaacaaaatttccttttttttaaaat
aaatgagcgggagaagagagtggggttgtacagaggtggggagagaggcagtaaaggagggaggaaagaagaggc
agatatcagggagagtggctccggaagttttgagttcagtcttagtcagaggtcacaccctgttatttctttggg
ttgtggacatgaggtgttaccacttgttatctgtcaccctttgaaatgatgattagattattggtttttattcat
agcttttcaacatttgggttctttaaaccattgaaggtttagtttgtatgggataatcagtgttattttagggtg
ggaaaatttccaccatacctgctaggagaaggggatggccaaagagtttttgacttacagaggagtttttttgtt
ctgttgttttttcctcattgactctatttcgctagctttgtctgagaggggtgcttataattctatatgtgacat
agtagctgagaggcaagctttggagttgatagacccaggttccagctctgctgctttgtgatttcaggcaagtta
tttattccatgtgtgtttcttttaaaactgggatgataataatagtacctgtttcccttatagagtaattaagag
gattgagtgagataatgcatgtaagcctttagcacagtgtctgcctaagttagcatccagtaaatgttagctgct
gcaataataagaaatattatagtattaagttattgttatttctgtgagggtgctggttttctgaatgctgagaca
ttggtgatctttgctcctgaacgtgttactatttggaatactgcctttattcagcagcagattcagttcagattt
actgaacacctactatgccaggaattgatctaaataagcaaaacaaaaatcctgctctcttgaaacttactttct
ggttaggagagacaaaatacacaaaacaaaatagatgatgtgtcactcaggaaatttggttgttggactttgtct
aagctttcattaaccaaccctagcagtcttaacctttacatactttttgattgttcctgttgctgtgcttgagag
tgtttggaggtaggggcaccctaaaagcgtatcatagactagctttattgtctccagcttcagcttgatcctaat
tttaaggcagccttaaatgatagttttcatcatttccttgacttactacttaaattggctatctcaaaactttca
gtccccaaaacttgtatttcacatcattcagagagatgttgctcctccccagctgctcctcttttgctcttctat
gccattttcttaagaggaggggatactagaagcatctttttagatttattttctgactagtctaactgtatactt
ttctttctcatattttgtgttttctaccctttatttattgttaactgatacatttaatttgcttattttctttaa
aatatgtgttttttttggtctctccaattaaattgtgaaagtttttaggtcagggaccacttttctattttgacc
ttgagcttactctcttgaaaaaaagataataccacccagggatgttgtgagacctcaaagagataatgtgtgaaa
ggggtcctggagccagatccttaataaatgttagtttcttctttgaaccactctagttgctgaaactgctgtgca
tataatcagtgcttaaaaaatgcttgttaatttatcttgaattgagtttgtgaactttcaattgtcacttggtgg
tagttttagtttttagtgctagataagattcaaaatactacttttcaagtgtcagatgataaatgctgggtcatt
agtggtagagctcaacagtgattaggagcaggaacttgagccatcattgtttgggttcaagtacaggctaccaca
cttactggttgtatgacctttggcaagttgtttaacctccctcggtttctttatctgtaaggaggggaaaatgat
atctacccacctcatggttacttctttgttagtattaaacgaagtaaatgagaagctcgaaacagtgcttagtat
ataacaatctataacagggtccccaggggacatttggcactatttggtgatgtttctgatggtcatttgggggag
tggggagagggaagggtgctactggtatctcgtgggtagaggttaggtataaacaaggtataggacagcatccac
aacaaaaaaattacctggtccagaatgccagccctgccaagcaacattgggaaacccagcttagttcctttgtat
aaggtactttgccagttagattctgtatacatgctgattattgataaagatgagtcacagacatggataggcttc
atttaacattcattttaaaagacagacatgtaagaaggttagtaggtttcatttctttttaaggttacagggaaa
gtactccaaaattaatctaaaagtccttacacttttttcagaaactgagaaaaggttccagatgaattttactaa
tttaatttttttatgacgagctattcatgtaaaagtgtattatgaatagagggcttatgagatatgagtgccgta
gtgtgtattcataaagtcaaacgtacacttaaaaaacaagtatcaaggtaaatggaatgatctattgggggaaac
cttggagctctctctctttccatatgttttattactcaccaagtcaagggctctttaacttaggatcttgttttt
ctgtgtacacttttcctgtttactgtttaaaattggacccttactaaaaatacagaaaattagctgggcatggtg
gcgtgcacctgtaatttcagctactggggaggctgaggtagggagaatcgcttgaaccagggagacggaggttgc
agtgagctgagatcgcgccattgcactccagcctgggtgacagggcgagactcagtctcaaaaaaaaaaaaaaaa
aaaaaaaaaaattggacccttaccattccttgtccatttcaggagcctcttaattgccttgcttctgttctttct
ctgtattcccacttgaattgactcttctttgctgggatccctttgggatcccttctagtccagatccaatctacc
cttcagagctccttccaccgtccccttatttctgggagagaattcagccttaataccctaaggatggaatttgaa
tgtactgacttttctgctatttacagggttgtgtattatctttggaagaattgggaagtcatatcattttctgta
tgttgtggttcaggtgacttgtttcacttgtatccaaacactggttgtgtgtccatgtatatacagcatgtatgt
actttaatgtctatttgtatgtgtgtgtacatgtatattctttcatgtatatttgtctctacaccttgctgtttc
ctctgcttggaatattcttttcttttcatctgggaaacatttgtctcttcagaccctacttaaatattgatctcc
taggaagtgtgtcctgattaatataactagaattagttatttttttccactggttacccacagtatctctgtggc
ttttatagcacttaacatccttatttgtatttttcttttctactgatctctcagttctttgagggcaagagtctt
gtcttagtcaacacctgctcccccaagtacctaacattcctgccacattgtagacatatagtatgttaacaaatg
aatgggagagacaaaaaactggaaagcttttaaataagttctatgggggttggcaggaggggagaccacacatta
tcttgtcacctttgtttccaagtgcttagtgttgtgcttgcccataggtgttcagcaaatatttgttgaatgaat
tagaagcaaatcccaggagctgataaaattttcatgtgaacttttttcttctgcatttctagaaagcatatccta
gaagtaaagtagcttagatattttggaacatcttattcttaaagtcgaccttcctatttttgactcagtagtgaa
actttgatccaatggatacttcctgaatagtgcttttggctgattcctttttcacaacataggtccgttctggtg
ttgagtttgttggaaagatgaatatgtagtatcatttttacatcatggaggagccatcattaagtaacagtgtta
aataaggactggtcccctttgcacatttgggcccaaattaagtgagtatgtactcttaagtatgggtaatgattg
aggctattgaggtgtaggaaagaaagaggacaactggaggtgaagaagtgacctactagatctcttttgcttttg
gtaatgattgattttttttttttttttttttgagacggagtctcgctctgtcgcccaggccggactgcggactgc
agtggcgcaatctcggctcactgcaagctccgcttcccgggttcacgccattctcctgcctcagcctccccagta
gctgggactacaggcgcccgccaccgcgcccggctaattttttgtatttttagtagagacggggtttcaccttgt
tagccaggatggtctcgatctcctgacctcatgatccacccgcctcggcctcccaaagtgctgggattacaggcg
tgagccaccgcgcccggccggtaatgattgattttttacccccagttatttttaaagaattggcaaggaaggaga
ggggtatgaatctagaagataagaatgtagattttgtaattcatgatcattattattaccatgtattgaatgttt
actgtatgtcaggaactgtcctaaggatttttacaaccgttatttaacttagtcctcacagcagttcatatgaaa
tagctactgatgttttatggcaaaggaaacaaattcagagaaatttccaagaacacatcggaattttgacttgca
gaactgggattcaaatctagatctgtcatactccatggtctatgcttttaatcatgcatgctgtctcttaagttt
ttattgaaaatggaaagttgtactgacataggaggggaaaatgatagaaaatcatcctattggaaggcagattaa
cagactcgtgaagaagcaattcccagttttatgtcattatacaaaataattgtgaaattgaagtccgtcatttga
attgatttgcattaaccctcattttttagtcttgctcttggaatcatcatcaggacaggtgtcccagatatgtta
gcagtagaataaatttatctaggcaaaatctgtctaagattatggagaaaactgattttatatgtgaaatgtgtt
gttgagtgagagttaaatcttttgaaggagattatttcaggatgagcctcaaatacattatttaggatttgaaaa
cctatggtttagcagaaaatctgagaatcaacatttgttagtctcttgggtataaattgcttaagtcagtgggta
tctgattattctttctttctttctttatttatttatttatttatttttgagacagagtctcactctgtagcccag
gctggagtgcagtggtgtgatcttggctcactgcaagctctgcctcccggattcacgccattctcctccctcagc
ctcctgagtagctgggactacaggcgcccaccgctacgccctgctaattttttgtatttttagtagagacggggt
ttcaccgtgttagccaggatggtctcgatctcctgacctcgtgatccacccaccttcacctctcaaaatgctggg
attacaggcatgagccaccacgcctggcttcccttatgtaatttcttaaactatttttgtttgctgatttggtgt
ttgtaatttaaaaaaaatttattttttcttcttttacatactttgtagaactattccctacccagtgagtttatt
tatagatatttcatatttaattaatactgtgagaacatagtttttatatctgtatttttgttttttcattaggat
atttaaaataatttttgtaattctgttaccttaaaaagagctaaacttagatgttggcctttaaaaatgttttgt
gaaagattttctcttggttccatttaaaatactgtatttgtaactatagttaaatacatttgaactttgcccttt
tcagaaattttaattatatgcctttataaaattttatcaaaagttacatgcaaatactaagtatagagtgctgaa
gctgagcgtgatggcttatgcctataatcccagttactcgagaggctgaggcaaaaggattgctttaggccggga
gttggagaccagcctgggcatcatggcaaaaccttgtctctacaaaaataaaataaattagctgggtgtggtggt
gtgcacctgtagtcctagttacttgggaggctgaggtgggaggatggttttgagcgtagtagttcaagactgtag
tgagccaagatcctgccactatactttagcctgggtgagagagcaagaccctgtttcaaatatataaataaataa
aatagtaggctgggtgcagtggctcatggctgtaatcccagcacattgggagtccaaggcgggtggatcacttga
ggccaggagttccagaccagcctggctggcatggtgaaaccctgtctctactaaaaatagaaaaattagctgggt
gtggtggtgcgtgcctgtaatcccagctactctggaggctgaggcaggagaattgcttgaacccgggtggtggag
gttgcattgagctgagattgcaccattgcactccagcctgggcgacacagcgagactttgtcttgaagaaaaaaa
gaaaaaaataagtattgagtgctgaatgatgttctataaaagattaaaaatgcctaagtggaactaaattctggt
aaatagaatttaggggttggggtagaaagaatcagataagtacttcaagattttcagactttaagtctaaggctc
attagtatcctctgacataatctactacaggtttcctttcttagaagtgaatgttaatatggcaccactatacaa
ataatatcgatatcggtatttgagtgtttattatgtgacaggcactgttctgtgtccttcatgtgtattacctca
tccagtcctcacgatagctctatgaggtaagtgttgttaatatccctattttatagctgaaaatttggcatagag
aagttaaacatggtcaaggtcccacctttagttaatagtggagctgggattgtttccaggcagtctggctctaga
gcctgggaccttaatctttattctatactgtctgtgtcatctgtaaatttatttcttatatattatttatagaac
agtgcctggcacacaataagagccatagaagtactggctattttcattggcatttaattatatatgaggttggca
gagcatgcattattattccatttgtttatttatactattaattagatagtagactttattcagatttctgtgttt
cctttttttaaatagtcgagtttaccgaagttcttagaaactggatgattgcctccaataatgtggccatgctag
atctcaaaattaacttttttaaaaaaaccacccctccaactttatttacacttccttagaaatggataaagacca
agattgggcttctgttttgatatgtcattggtttagaaaggcagaaacagcctggcacggtagctcatgcgtata
atcctagcactttgggaggctgaggcgagtggatcgcttgagctcaggagttcgagaccagctggacaacatggc
gaaaccatctctactgaacatacaaaaattagctgggtgtagtggcacttgcctgtagtcccagctacttgggag
gctgaggcaggaggattgcttgaacccaggaggtcgaggctgcagtgagccaagatggtgctactgcagtccagc
ctgggtgacaaaataagaccctgtctcaaaaacaaacaaaaaaagcggaaacaaccatactaagtttacagtatt
gtttgtgtgcctacttggtggaagaaataaattcttagaagttacactttctttaaaaaattaaaaattaaaaac
tgtgcagtgttgagattagtattaccgcttatcttgctgacttgtgaaattgaattacatcacttataataatgt
gaagttttagaaattgttttgatgataaagagtagcagcctttaagagaaataatataaaccatgtttcctcaaa
tctaaatggtcatctgtatggtattagttttccatcgctctacagaagcagtccccagcctttttggcaccaggg
accatttttgtggaagacagtttttccgtggatgcggggaaggagaggcagtggggaatggtttgggatgaatca
tcaggcattaggttctcttaaggagcacctaacctagatgccttgcatgcacagttcacaatagtgtttgtgctc
ctatgagactctaatgccacagctgatctgacaggaggcggagctcaggcgataatactccacctgcagctcacc
tcctgctgtgcggccgggtttctaacaggccacggaccagtaccagttgtggacccctgctctacagcaaattaa
cacaaatttagcagcttatcttacagtttctattggtcaggaatttgggtatggtttagctgggtctttcctcag
gttctcactaggctgaaattaaggaatcagccggggctcctatctcttctgaggctctgggttctcctccaggct
caccggttgtgggaagagttcaattccttgttcttgtagatctgatgtccctgtttgcttgctggctgtcagcca
gggaccactttgagctccttagaggcccacccccgccaccatgttcttgccatgtggccaagttattatgttgtg
tatgactaaggaagaaaaatgctggattaaaatataagacaaattcttactttatgtatgactaaggaagaaaaa
atgttgaattaaaacatgatacaacacattattgcttggacagtgccttgcaaaaaaaaaaaaaaaagagagttg
tggttattcttccagccacaatatctgcttcagaaatgtcaaatttattttctactgttgtgtttgtgttctttt
ttgtaaacaacttttttgtttcaatgctgaatcatagtattttacaggacatttaaaattaagaagtaagcgtga
cttcttattgtactactaaatgtacagaatttagtaataattatggtgatataaactgccattacccaatttgca
agtgtacagggaatgacagctatgttgtgactgccatgggcaaatggtgatttgtaaagcattgttgcttttttc
cccccattttaagttgattttattgagatataatttatatacagtaaactacaagacatttctgtcaccctcaaa
agattcctcatgccccttcacagttaaatccttccctgcaccctcgcttcaggcaatcaatgatctgctttttta
ttactacaaattagtttgcattttttagtattttaaacagtgaggaaaaaagaggagatggagacaggctatttt
taatttttatattttcttctttgttcttagagaaatcctagtttctttattaggcattaggtttattccttcagt
gtgggggtaaagtgattcttcttgaagctttctaagtatgtttcctagatgttcttgacctctgttttgcctaag
ataacttttctccagcatacccagagttggctctcttaaaccatacgtgcttgtgtagctccctcatccatattt
ctacttattgtcttttcaggaccgtaaacagaggtatttttgacttgacattctcagctcctaaataatcttata
gtgttactgaggcttttcacctgtcacatacccccgtcctagtctctttttgtgttgctgtaaatacccgaggct
gggtaatttatgaagagaagaggtttatttggttcatggttcctgcaggttgtataagaagcatgatgtcagaac
atctgctttctggtgagggcttcaggaagcttccactcatggcagagagtgaaggggagcaggtatcacatggtg
aggcaggaaaggagagaaagaggagagggggtgccaagctctttttatcagttctcatgagaacttacagagggg
aaactcattcattaccttgaggatagcacaagctgtttatgtggaatctgctcctatgacccaggcacctcccac
tgtgccccaccctcaacattggggatcagatttcaacctgagatttggaggtgtcaaatattcaaattatatcat
cccctagagaacatacctccccacccctgagaatctcagaatttcttgttgagaattcatattacagtatgagtt
gtaggaaacatgaaggaaagatttcccttctagtcagttcttatatttatcacttgtttcatgctccttggtgtg
tatagtgttttcaggtgcaggaaatgctctgggaagaatgttgcttcttagaacgtaagagaaagggaaccccaa
gggaagactcaaatgaagtccttaaaaagcccacttagcttcttctgagacagcattgtccaatggaatgcaagc
gagatagttaaatttaaattttctggtagctatgttaataaagcaaaaaaagaaccagccaacattaattttaat
aatatattttatttaacacaatatatcccaaatgttattttaacatgtaatcaatatgaaaaccatgaagacttt
gatatttaaaattcttattttaatactaaatctttgaaatcagtgtatattttatacttacaagacatctgaatt
tggacttagtcacatttcaagtaagtgctcattagttagtgactaccttagtggacagtgtagatgtagattatg
aactttttaggaaaagacaatagcatgtggactccacaagaaattgtctttaacaactgtttgactctcataggt
tagggctttagtgtttcagaatggtttctatggttgagatgtaaagttttttatttttataaaggtaagtacatt
tttatacattttcattatacaagtaatgtcattgttgaagagttggaaaagtctgggtaaaagacaacaaattgt
cctcacttcctcagtccagagataaccaatggtaaactttattgaatttcctctttgggaatgttgctttttaaa
atcaaagagaaaatagatttcttttttgtcataattccttcctgttcacattcagacatacattattattgacaa
ttcttttacctctttgtttaaatttatgtattctttttctcgggctgtatcctccaatagggtctctgtgagtgg
agtactctggagcacagtaacctgaatggtgcttccctggattagggttaacaatagaaacttcaatggctttta
tgtactcattgctaaaccagtgtagttgctttctattttaactatctgcgatctctttgcatatcctgcagcttt
tttagttattgaggtataatttgcataccagaaaattcactctattaaatactcaattcaggccggcataatttg
cataccagaaaattcacgctattaaatactcagttcaggccaggcgcaatggctcatgcctgtaatcccagtttg
ggaggctgaggtgagcggatcacctgaggtcaagggttcgagaccagcctggccaatatggtgaaaccccgtctc
tactaaaaatacaaaaattagccgagcatggtggtgtgcgcctgtagtcccagctatttgggaggccgaggcagg
agaatcgctggaacctgggaggtggaggttgcggtgagccgagatggtgccatttgcactccagcctgggcaaca
ggagcaaaactccgtctcaaataaataaataaataaatattcggtcgttttcattgtattcacacagttgtgcag
taattatcactattaatccagaacattttcattacctcaaaagaaccctgatgctcattaaggagtcacttccca
tttcctctttgcgcagtctcctggcaaccactaatctacttcctatctctatggatttgcctattttagacattt
cttataaatggagtcatacagtacatggccttttgtgtttggcttctttcacgtaacacgatgttttcagtgttc
ttctgtgttgtagcatgcatgagaacttcattctttttaggactgaatagtattcagttgtatggagggtaccac
attttattcatcagttgatggactttggggttgtttctgctttttgagatcacattcattcttcaagaacattaa
tttcttaatatcgtcctccattcttatcaaagctttcagtggtttcctgttgtctataaacataatagaaaaata
tttagtttgcattcagtcttacaaagtgtaacttcaagttactttcttttccactcttattttccatcttgttgt
tgtttactgtgtctaaaatatgtgctatatatgaattataaaattatgtagacaatatagataaccttgtcatct
gatctaatttttaaaaagtgaatttgaatactgccaaactaaggagtatgtgtagtatggagaatttgagaaaga
caagtatcagtaaagaagaaacagtatagtaaaacaaaatttttttctgccttggtccattgaaagaaatattta
gtttcatataatgtgaacaaaaaccttaattttgaatgaacatttgatacataggaactcattcaaatcattgat
tttctggttaactttgttagtagtttgtttttttctgagtcttttgttttaatccagatagtcagtctttttctc
ctttttacatgcttgaaatggtaaaaatgaaatatttttacttattcttaaaaatatttccagaatgtcttacac
acacatacacttagaaacaaatacaaatatatagtgatattctctcacattttacagagaaaagcgtagtataca
tactttgttttttaatttcacagtagatcttagaggtcttttaatattagcatactgagaactttctcatttttt
tcataatagtattgtttattccattgtgatgaatatgctattgtttttgaaactagtcccatattggtggacatt
taggttgtttccaggtccactattacagataatactacagtgaataagtttgtacatgtatcatttcatatgtat
gccaatatttctgcaggataatttcccaaaagtgaaattgctgaatcaaaagatatatccacctgaaatttgatt
acccatatttttaagcataaaggaagagataaagggaaataaatagaaaaaaaagaaactatagattttcaaaaa
ctttgtttatagatgaaaaaatggaggattagaacttttaagtaattgtccatggtcatgcactccacagaggac
tgggcatggtgggattcggtggggggggcggggggctgtgtcaggatgctaacccaggtgttcctgattttaaat
aagctgctatccttcctgttatattcatactgcatgttatgagcaaaaggggagagaagtcgccaactttttgga
gaaaaagaataaaatcccagactttatttttgggatcatctgaggtgggaagatggcttgggcccaggagtttga
gaccagcctgggcaacatgaagagaccccctgtctctacaaaaaaaaaaaaaaaaaattaaccgtggtgtttgag
gcttcagtgagctgtgatcactgcactctagcctgggtgacagaatgagatcctagtatatgtaggtatttaatg
tatatcgaaggcagcatttcagattggtgggggagaagatggattatttggtaatttatattgcagcaactttct
attcatttggggaaaaagccctgtcccttgctcttactctttacataaaaaataatttaaaaatgaaagcaaaag
gtaatatcgaaggatgtcgtgaggttagccttattcaatagactgacctcagtgagtactgaactcatgaaagtt
tcatttttttcccagtcactgtcttttgcattttccttaattgatttttaacatcaccttggattaatctcatag
ctttcccaatatgtttttagttatgaaaagtctatttctcctcttgccccgtattaggcttgacctgtggctgga
gagatctgtggtactgtttcatatgtaccgccctcctctttctctcctcccttttttacttgttctgaaaacaat
gagtaaggttaaaaaattatttttcacaaacatgaaaaaggttgataatacgtaagttttgaatatgggggaaag
ttactctcatactgttgttgggagtatacatttttgtggcctttttggagagcagtttgacagtatttatcaaaa
taaagtacacaggcatcttgaatcagcagttgttcttccaaaatttagtagtacagatatactgacataacttta
cttgaaagaagaatcctttttataacttagtgtttattgacaagagaattagcatttagagtcatttattattct
aactcttctaccaaaatggtgagatgtgttgtaatttactggacgtttaggttgttcccaatttttttgtaagta
tgaaaaatgctgcagtgaacatctttgtctgtgtgctacccagtcatcactgttgagaatgtttcttaatggcta
tatttaggtaagtgttatttccctttatttcccctcttatactttaaggagcacaggagatctcagttttaatcg
cagctcaattgcctaaatggatgtgtgacttaaaacaagctctttaaccttactgagcaggaggttctgcagata
tgggcctctcagactattttatagcagtaataaatggactacctattgtcacattcttcatgatcctgtagggag
tatgtgtcagtaggacaacccattgagtgacagtcctaactttcttggttcctctccctcattgtgaggtcattg
tttcacaattgtatatacttactgattcaaaaaacaactccaacacttattgaggggtagtactgttcccagttg
agaattactgttgtaaaaattttggcaggaattatataagattattcatgattgttatcgacttatctttattcc
ttcatttttttttaaagcaggaagcaataatagtttacgttcgttaacctcaatatctactctaaaacatgttgt
ttttcttgttattaccatgaaagtctgggttaacaggagacgagactagcttcttcacagatacatacattaatt
tgacattatagaaaaatgagactgtatcctgacttgccagtttggttttgattggtggtttcaccttttttttta
gactcaggaaaacggtggcaaaaatagtataaagggtaaattctcatatgcccttcattcaagttcccaaaattt
aacatcttatgcagtgacagaaaaattatcaaaatcaggagattaacattcatgtaatactgatagtctacagac
tttattcagatttcacccattttctcaccaatggcctttctctggtccaggatcacaatatgaaattattcacgt
gttgtattttagttatcatgttttcttagttttctttaacctgaaacaattccagtctttctttgccttttgtga
ctgttatactttggaagagtactggtcagtaattttgtggaatattgcacaatttgtgtttgtatgttttcctat
gattcaattaattttaagcatttttgattaagaatactgcaaaaatgattttatgaatcttatttagaggcacat
gatgctgatttgtccagttatcagtgatactaattttcagttcctttcgttaaggtggtatcaggtttgtccact
ctagttgctttttttcctttgtgattcataagtagtgttgattcagattctaggttaagaaaaggaaaaaaaaaa
gtatcctatgggaaggtacttaaaaataagtactttgggaccaggtaattaacatttctcaccatactctaccaa
tttttaacttccttgctgttttttttttcttttttttttttgcagtggcacgatcatgagtcactgcaagcctca
atctcctgggctcaggtgatcctcctacctcagcctgttaagtagctgggactgcaggtgcacaccaccatgcct
ggctaatttttcatattgtttgtagaggcgaggtttcatcatgttgtccaggctagtctcgaactcctggactca
agccatcctgtgttggcctcccaaggtgttgggattacaagcgtgagccactgcctggccccagactttaacttt
tattgatgattttgtcatggtgattttctgtttccatcatttcttctgtatttacattagtggtttttggtaagg
aagaactttcccttgttatgtatttatttaatgttttatgtatatcagaatggactcgcggattttttttcccct
cttatatcagatggacttacagattcttctatggatttcatctattatcatcattatttattttggtgctctaat
tatcccaggtttggccccatgaagcctcttcaaggaggttcctggtccttttgacatgtctccatcattttgtga
gggcttaactttttttttttctttttgagtcagggtcctgctgtgtcactcaggctggagtgcgcagtggtatga
tcatagctcactgcaacctggatttcctgggctcaagtgatccccctgccttagcctccggagtagctaggacta
caggccaggtatgtgccaccacacccagctagttttcgccttttttttttttctccaatagagacagagtcttgc
tgtgttgcccaagctagtcttaaactcctggcctcaagcagtcctcccaccttggtctcccaaagtgctgggatt
ataggcatggaccaccacacctggtcagaaggcttaactttctagtagcgcaaaatgttctagtctcttcttgtg
cttttctctgccccagccatgaaatcagccatttcaccaaggaatattgattccttttactggaaatcggaattc
ttccattccccctgcccccagtacttgtatcttaccatctttttaaaagtctttgtgtcttagaaccgtgttaga
tgatctggacacattaccctgatgtacagaaattagtttatacacggtaattctttttgtagcagactgtttaat
ttgtagcagattcattttgcactgattaatcctggaggatttcttcacaagttacattgtgtcaaatatgttatt
tcatgggaacacaattgaattttttccttaattttgttcggtatttgataataacaatttaaaaaacttttctaa
gagcttccttctaatattatatgatataggaatgtttacatttctcatttatttatttggtgtagtgatctcaga
agtttttttaaaagaaaaatcttgactttgtctcaggtactctcagttcacttccatgtgggagggtcatttgcc
atccatccagattacctttttgattaggcttttcagtgtctataaatatttcggtcttttctgatcatttaattt
ctaatacatgcatatgctcctatgtataataaatagacaacttcaaatttgcagtttctagatggttggaaaagg
gaaacattgtggtgtgtaatttatcagccatcagatcctagatatttgagattttaactaagcaaggtattagat
agaccatgttgttttggcttcacagaattcattcatattgtgcattacacaatcagtgtgcatattgcacattga
ttttatctgtaagttgtctttatcagtggttctcaaagtgtggtcccctgctagtatagtatcagcctcacattg
gaactggttagaaatgcagacttctcaggatccacctaattgcagtagttaattttaacaagcccttcggtgatc
ctgaaacatgttacagtttgagaaacactgctataatacgtttcatttaaattgtttcaggttgtgggggtaggg
aataagactaccaatttattcatcttctgtgcaatattacctgtttacctaactcttagagatattaagatattt
tgaagaatgtgtcccatgagattataatggaactgacaaattcctattgcttagtgatatcatagctgtcatgaa
gtcttagtgctgtaccttactcatgtgtttgtggtggtgatggtgtacacaaatcttctgcactgccagtcgtct
gaaagtatagcacatggccgggcgcggtggctcacgcctataatcccaacactttgggaggctgaggcgggcaga
tcacaaggtcaggagattgagaccatcctggctaacacggtgaaaccccgtctctactaaaaatacaaaaaatta
gctgggtgtggtggcgggcacctgtagtcccagctactcaggaggctgaggcaggagaatgacgggaacctggga
ggcagagcttgcagtgagctgagatcgtgccaccgcactcccgcctgggtgacagagcaagactccgtctcaaaa
aaaaagtatagcacatacaattatgtacagtacctaatacttgataataaaggactgtgttactggttgatatat
ttacaatactgcactttctgttgttactttagaatgcactgtttgaactttaaaaaaataaattcactttgggag
gccgaggcggacgaattgcttgaggccaggaattcgagactagcctggccaacatcgcaaaaccctgtctctact
aaaaatacaaaaattagctgggtgtggtggtgcacgtctgtaatcctggctacttggaggctgaggcacgagaat
tgcttgaacctgggaggcagaggtagcagtgagccagtatagcgccaccacactccagtcttggtgacagagtga
gactctacctcaaaaaaaaaaaaaaaattgcaaaacagcctcaagaaggcccttcaggaggtgttccagaaggca
gcactgttgttgtaggagattacagttccatgtgtgtgattgcctctgaagaccttccagtgggacattatgtgg
agatggaagacagtgatattgatgatcctaatcctgtgtaggcttgggttattgtgtatatttgtgtcttaaaac
agttttgaaagttaaaaaaattttttttaaaacaggaaaaaggcttataaaataaggatataaggaaagaaaata
tttttatatagctttactatgtgttttaagctaagtgttattacaaaacaggcaaaaaattaaaacgttcataaa
gtaaaaacgttatagtaagctgaggttattacggaagaaagaaaaaattttaaaataaacttagtacagcctaag
tgcacactgtttataaagtgtatggtagtatacaataatgtcctaggccttcatatgcatccaccccaactgact
catctagagcaccttccagtcctgctagcttaatttatgatacagatgcaccgttttacattttatataccttat
ttttaatataccttttatatatttagatgtgttaatatcattgtgctacagttgcatacagtattcagtacagta
acatgctgtacaggtttgtaacctgggagcaatagtttataccatggagatttgtgtaaaagcactgtgtaatat
tcacacaaagaagaaattgcctgacacatttctctgaatgtatctttgtcatgaggcgatgcgtgactgtataat
ttgagccacacatgttattttaaaaattgcaatacccttattaatgtaaaaagcagttgaattaattttaagata
tttagcctgatggagccatgatattaccatttcaacataatttataaaaaaaattgttattttagattctctttg
taccaagtttttgaaatctggtagatattttatatctcaatttcaacactaactttttaccggaagtaattgatc
ttcctttagatttcataaagtttatggttggaaaagtagattcacatatctaaatcgtttcagacacacttaagt
atttcgctacctgaattaagtaccaaaaaataatttttctgtaatatttgcatctaaattggtaaaactggttca
tgttttttagtagaatgtagtagactttgaagcaaaactgtatccatttcaaaattgagctactgaagttaagca
aattcatgaactcttgtgtcaactccgtattactgacattatattcaaaaggagattggatgttaggcaaatcta
tattaaataaagtgtttttcaatgatttttttgtaatttttgcagccactttgcaaaacactgtcaattaaaatt
tatatatgttgatttatgttagaacaaagtgtaaaatcttttttattggtttgtgttatgaaaactttaaatttc
aatgtaaatttataagaaagttcttataaaatttctggcataccttcttccagtttctttttattttactttttc
ttgtgttagcctggtcataaataaactctacctttctttggtgcttcaagttaagcttgttcataggcattgaga
tgtaagtgaggaaacaaaagaatactgccatttttgtcttttattacttaatagtgaaaagcatctctttctaaa
atcagagttgacattagagtagatctttgtgaagttattccactatacccagtgagcaaagaacacagatgtttt
aaatgtattgttttttatttttttaacatttccatgaactggctaggattcatatttttggcatgtacatactga
acaattcaactctttacatagggcttttcatagtctgtttcagaaagctgaacacagatattttcaatgtgtatc
atacagtggaataaaggaataggagaaacatcaatttttgcttttaaaattcctaacatagctggagctgtctgt
tgtgatagaaactaattgtttaatacctagctgaaattctttgacagaggtaaaggattaaaaaatatctatgcc
actcttgattttttttttttttttacagctacatagtgacaactttttcttaagtttagaagttctttcaaataa
atttcacctaaaagatttaattggggccgggcacggtggctcatgcctgtaattccagcactttgggaggccaag
gcaagtggatctcttgaggccaggagatcgagaccagcctggccaacatgacaaaaccccatctctactaaaaaa
tgcaaaaaattagccgggcgtggtggtgtgcatctgtaatgccagctacttggaggctgaggcaggagaatcact
tgaacctgggaggtggaggttgcagtgagccgagattgcgccactgcactccagccagggcaacagagcgagact
gtctcaaaaaaaaaaatttaataaggtagtgtctcttatatcacatggctcatctaaagctaaagggaaatagta
atttttcacattttgaatctgttgatttttcgtattattagaaaggtcttccatatgatggatggtgtcttaatt
gaatatcttccacgtgttgtaaagtatctattttagtattcctcataattttctaacaaaatttctataattgaa
ataatttctttaccatctctttatgtaaatgtgattttctttcttttggcgcaagaatttaagccagtttatagc
tgaccagagttacaagcccagatttgttaaaaagcttttaaacacgtttgttgtacatttacttctgacattgtt
tggctaattttgttgcttttcttctgattgtagagcagaaagttcttataaaattcactgtgtatttgttgaaaa
tgtctcctaatattgtctactttacttgtaaaactttaaaacccaagaaatagcttttaattttgctctgtcaca
gctaattgtaattgtcattcattagaaaatttctagcataccttcttccagtttctttttactttactgttctgg
tggtagcattagcttctgtatctctacttgattgtgtattgctgtaatgccttctttttacttttaaaaatgtgt
cctcctcttgtccattcatttttaaagtaagaaaattaattatattcaaaatattaaaattaaaaaaaataaaaa
gtattgcgattgagttaccagttgtgatttacataggcatcactgcaacttgtgttatttgtgaaaaacgtattt
aagtaaacgcagtacggtgttagattgatgagtagaaaaatactcattctaactgtaaattagttaatttttact
gactagatcagtatttttatgtgtaatactagaaatgactcactgtatcctgagatgtggagtataatatgcagt
acagtggtcacagtgaaatgtagtactgccaaaagaataaaactttctttagtagaaggacattttacacagctt
cagcttttaaatttaaattaagtacaactaaataaagctaaaaactcaattccctggtaacactagccacatttc
aagtgcttagtagtcacatgtggctaatagctaatatattagaaattaaaccttttgagagttggttatgtatgt
gattaaagtattttggttacttgggactagagattacagtcatttttgatcaggctgatgtcataggaacagtac
caaggggacttctgaatcaagcaccctagaaagagctacttagaactacttgcattttctttgtggcatctctta
taggcatgaaaaaatttcaaacatttttcatggataaaaggatttaatcagaaaagcatttggatatacatattt
gaaatcatagcttgcctgtatgttactagagtagttggagagtggaaaagtatctatattctaaagactgtttca
tcatttgggtgaaaaaccaaaaaaagagagtaattttgtttaatagctctttcacaaataaaaaaaagaatgttc
atctgtagagacctatcgagatctcatcaggtttacaacaaactttagattagcaagctcaatttcagttgacat
ggattgtggaaagattttcagtggagctcagatatttaatatcctgaatttggatgaatatacaatttctaaaat
taagtacttcaactcctctgtggttttaaaaccagtagtggccatatcttgactagttattaaagcaccaacctt
tttttctttattactttgcttttatcctttgagcaagttaggcaataagttctccatgtttttgatttctacaga
agtaaatgaatgttattgctgatgtttatttcattctgtcttgatctcatgatatgagaatataaggagatgtct
gaaaacttgttttaaaaatcctatgaagcataagttcatgaataaagaaggaagtggaacttgtcttgagagaat
ttgcctatttagatggtttccaaagtgcattctgctagtcactagagatggtccttgaaagagttctgtaattaa
gtttgaagaaacactgtgtaccataataatggcctattgaatattttaccatgcacattaatatatcaaaggctt
tgagatatcttacaagaaagaaacttgctaaagcttgtcttcctgcatgtcttaagcttcttccacagtgacttt
aagtcggacttgtctatcctgtgagaaatgctgccctctagaggcacaaattcaattgctcttaaatttgagtac
cataatgctgcctgatcttagtgtgaagttgtgataattaatattaatcaaatgcatggtaacaaaaccaaatag
aagaaatcttatgttgcttaattttaagaaaatatgaggaaacagaattgcaatttataaatacccactttttaa
attgtacatgtgatatttttggctgctaataatcttttgaatacatttctgtatatggggaattgcggacacata
gtacttcaagtctccaaactctgagtctctcctgctagcagaagccatccttccctgtgttaacaaccttgctta
agacagttaccatccatggggaagctagtttcctcataccccttcccacgatactcatgtttccatacctgtaac
tggagtcagactatatcatgctgcacctttgcagtcacaatgggaagaaaaggattatatgtaatttttttaaag
ttttttttttttttttttttttttgctgacaaagcagcaaaaccttcaggaatatgtacagaaatgggtgatgat
gatgatgtgagagagaaagaaacatgcttttgcatttggctgaatgtattggtatggtgcagttatcagagattc
tgaattcattgttctcgttcaagtagccaggagtggttctaattgtttgctcctttggtctcagcattgacttat
actaaatgaagttgaatgcctaaaaactcatagcataatatagaggaagaagtcaaaagattaaaggaaatagaa
ctgatggagtggatttatcactgtgaccattcacttacctcccctattccctctccagtattctctactaagagg
tctggaagacacttcattcaccaacctaatgagaaatactttgagggcagtgccagcatatatgaaaagtaattc
aattgcttttgtctgtaggctggaggagatactttcattgaaattgtcttttttacttcagggggtgtggccaga
ttctaggtaaggtccaggcatcattttcataaggtagcttgacaggcatgatgatcagagatgcactgacttgaa
ttctttttggctgtgactaaatgatcatggatccctacgtctaaaatagagaggaagcctattaagatcctactt
tatctgtatgagcaaaaaattttaggtctcataggagaaggccttacttgaactaccataagagtgagttggcca
gcctgggcaacatggtgaaaccctgtctctacaaaaaacacaaaagttaacttttgtgttttgggcatggctgcg
tgagcctgtggtcctggctgctcaggaggctgccccctttttgcagtttacttcatgggttatatgagaaaacag
gaaggagggggatatcaccgagagattctgatattggagctgtgtgcaataactgataagtctctggttcttacc
tccatatgaggaacagagagagtcttgtttgtacaaggcttagttataatctgtcaggtttgaggatcttgattg
gaagcatagacttgatctgtatttgtggtttctcatttcaaatagcctctcaatgttggataaatttgcatgttc
atgttttatagagtcagaactcaatttcctagtattccttgcagctagggcacaagcatatgacctaggctttat
tgatcagatgcatctgtgtaagacatgaattcagggtcagagaaatgcagggtaacagctcttgcctggaattgc
tttctaatgtgcatgttgagtgacaagatattgagctgtttagaggtagcagtgacagagatactagtgagcctt
gttgtcctaggctcagctttcatggtactagggctgaaggatgggaacaatttggtggggagacttaggtatttt
tcctggcatcatatctccaagtggattcttcagttctctgataattcttggaatcccctaatatattaataaatt
tattttctcttgaaaccacctcaaatagattctgtttatagcttagagccctgattgatcgtctaactttaaaga
ttttatttatcgtcaaaatattttatttttctaattgagatctaaaaattgtcaagcacttatactctatttttt
cccttcctagagctctgcaatcctttgctgtcattttttcttaccaggaatgccaatttttgtaccttttgaatt
gctttttcttctttgctacttggcttgagctccactattgttatgaaacctttcttataccatcccagcagcctg
atggatttttttcttcctctgaattccagtagcacttgtttagacattaagtgcaaggtgtttttttgttttgtt
ttgtttttctgtatgcatcttagcttccgcactaaatgataagctccctgaaaactagtactatgcctctttata
tcgcttgcattgtccaacagaatgtcttgcacctattagattcaaggttcagcacagtcaccctcataatatata
gtgatgagagttgctgagaatcacagtgatgccaggctgaatgagctgagataataaactagttagatgcatagt
ctggccatgaggcccaatttccatcttcctcaaggagtctgagacttgcctctcagtagttttcgttagtaggtg
atcatatcttctgaattactgtcttcttaatgttttatgagtttttttttgccttgacaaaagccttctatttct
ctcatgcttcctagttccttccattgtgccctctgcaatttcttttcactaagcttactatttgcagtctcttaa
aatttgtgtaaattatgaaatcaaagatacaaagaaaggacagaaagtagtataacaaacacctgtgtatctaac
ctcagggtcaagcagatattaaccttttgcctcgtttgatttccttttaaagttcttccctccccatacttttct
cccttctttggaggtactcattatcctaaagtccttacgattatttccctgctgtttttgtacacataaaacatt
tacacaaaagatgacctgtgtttttttctagatgtgttcatattgacatgtgtagatttagttcattcattgtaa
ctactgtttagttttccaggttgaataatgctaagttttatagagataatgcagttctaccttttggcaaataat
attgtaatgctcgtacataggtacttactgtaattttatctaggatagatacctgaaagtagaattgctgaatca
cagggttcaggtgccactttatcaagtattgccgagtccagtccaaagtggagctaccattttatacccattaac
atgtttccacacttttttttatcaacatttaattttatctagctatgatttttttgcctatttgaaagatgagaa
atggagtttgatgattttcattttcattactttgatttatagtaaagttgaatgtctttgcatagtttttgatca
ttctagtttcctattctgtgaatcttttatgttctttgctgatttttttcctattgggtattgaaccttaaattg
atttgtggtgattctttttataatctggatattaatggttttggtaatatgcattgcaagtatctcatagtgtgt
gtgtgtgtgtgtgtgtgtgtgtgtgtggtttttttttttttttttttttgagatggagtctcactctgtctccca
ggctggagtgcagtggtgcgatcttggctcacacaaattctgcctcccgggttcaagtgattctcctgtctcagc
ctcctgagtagctgggattataggtgcccgccaccacacctggctaatttttgtatttttagtagagatgggatt
tcactgtgttgtccaggctggtctccaactcctgacctcgggtgatctgcccgccttggcctcccagagtgttgg
gatgacaggcgtgagccattgtgcccggccttcgtgtcggtttttaacattgtttaaaagtttcagatttactac
atgagttcatttgtagaatctctcttctgtatcatagcttctttggtctgctttcatgtctttacaatattgtgt
taattttaatagttttgatataaatcttgttatctgataaggtgaatcttctctcaatgaaaattttgggtcagc
taatcatatttcaccctgaccctaatgaaaatttaacagaaaacaaaccctgttggaaatgtgattggaatctca
ttgaatttacaggttacagtggggatagttgtcacctttacattggttgactgtcattgatatttctgttcaata
cagttctatttttttcttttttctttttcttttttttttgagatggagtcttgcttttgttgtgatctcggctca
ctgcaacctccgcctccctgtttcgagtgattttgctgcgtcagcctccccagtagctgggattacaggcgcctg
ccaccatgcctggctgatttttgtatttttagtagagacagggtttcgccatgttggccatcctgaccttaggtg
atctacccacctcagcttctcgaagtgctgggattataggcgtgagccactgcgcctggcccagttccatttttt
tcttgatagaagtcttcattagattttttttttttaggtttttatgatttttgttaccatcgtgaatggcaaatt
tttataaaaatactttttgttactggtagctacaaaccaattagtataagagacaaattgctaagaagcaagttg
ctgtttgtatcttgattttgtatctctcaatcttgctgaattcttagttgtttttgtttgtttgtttgaggtggt
gtgtcttactctgacacctaggctggagtgcagtggtgtggtcactgctcaccacagccttgactgcctgggttc
aagcaattctcccacctcagcttcccaagtagctgggaccacaggtgcatgtcaccacacccaaccaattttaaa
attatttgtagagatgaggtcttgctatgttgcccaggctgctctcgaactcctgggattaagcgatcctcttgc
cttggcttcccagagtgctgagattacaggcatgagccattgcacctggcctaaactcttacttctaatgataga
ttctcttggatacctcatgcagataatcatataagtagtgaataaagatggtttttgttgctttctaagcctatg
tctttatatttttcttctgttattgacctatgtaggaccactttactaaaattgcagcttttcctgggccttggc
tttatgtgtgtgttaatgtatggagtagaaaggggtatctcagaactgaagtctcccactaagggccaaaagtct
catttctagtccctacatatgagtcaaaggataaatataggttaagactagcaacccattgtagggaagaaacat
tgccaacttgtagcctaatatatatatttttaatatgttactgttttttccatgttactggcacacacttaaaga
tttctgtttcatggtggctaaaggattaatttaaaataattttttttttcctttttgagttgagtttcactctgt
tgcccaggctgaagtggagtggggtgatctcagctcactgcaacctctgtctcccggtttcaagcgattcttctg
cctcagcctcctgagtagttgggactacaggtgtgtgccaccatacccagctaatttgtttttttttaaattttt
agtagagacggggtttcaccatgtaggacagggtgatctcgaactcctgacctcaggtgatctgcctgtcttggc
ctcccaaagtgctaggattacagacgtgagtcaccacacccagcctaaaagaattattttgatatatcctttctg
catgaaatttctgtgggaaggcttttcaggtgatctgatctgatacagtgttaggactggaagtatagcttatac
tatataaatttccttaattgaaatttcacattatttaggaactctgtaccctgaagctgtctcactctggtattt
aatgtcctacaattagtttaagagagcaagcttgggtaagtttcttaaaacttccttatatttgtttcttcatca
atattagtagtatagtatcatctcctatcttatagattattataagaaataagttaataatgtaaagcatttaga
acagtattagttattggtctcaacctccttttagctgttatctacagcctttctggtagattttacttattctgg
gaagatagtggtgtattcctcataatctccttgtacattccaggtcctacaataattctcaaacattttagcatc
cttacatagactctccaatatcctggtcttatttccctgatctcctcaaatctactgatttttctcttcctcaga
caagtgatttttcacttatttggtaaaatcctaattctggattaactcggttatcagattctttcattcctatac
cacctgaatgctgcagtaaaaagttacctcaaaggacatttaaaaaaaattataaattcatgattgttgatcaca
gaccctaatactgcatgggaatccactgcattatccttgtcagctagtttcctcatctttaccatgactgtttca
atcatctgttctcctcagacctccaacatgccctttctctttctacagaaaacttttcctgttactttccagtga
aaaccggagtacttggtgcaacccttagttgcccactatcttttctatattttctttcttcttaatgttaaaata
gaggtagtcttgctttctatctaaggctttctccctttctctgcagtaccttatgctgttgatgatttccctttt
ttttgtatgtttaacttttctttacacttgattcttctcgtcagtatttaaacacgcttaattcctgctattaaa
aaaataaaaacaataagcattaatgggtaccacattactgacctttctgttctctttcctagccaaatgctagaa
attattgtttccatttctcacctttgactcacctgtttagtttattcattccactgtaatagtatttgcttatga
taccaacaacctctctgttagtaaattaagtgaatatttttaaacttggtcttatttgatctctcacttggtgtg
ctagggcttctataaaataccacagactgggtggcttaaacaacagaaattaattttctcactatgtggaggctg
gatgtcaaagatcaaggtgttggcaggtttgttttcttctgaggcctctctccttggcttgcagagggtgccttc
ttgctgtgtcctcacttggtttttcctctgtttgcatgcatttctggtgtctgtgtccagattggattagggcct
ggaagatgaatttactcacctctctaaaggccctatatccaaatacagtcgtgttctgacgtactgggggttagg
gcttcaacacgtgaattttgggggagacagtttggcacataatctcctagcaggtttaaacagttttgaaaaact
atccttggctggacgcattggcttacacctgtaatcccaacactttgggaggccgaggcgggtggatcacctgag
gtcaggagttcgagaccagcctgaccaacatggtgaaaccctgtctctgctaaagatacaaaaataagccaggtt
tggtggcacatgtctgtaatcgcagctactcgggaggctgaggcacgagaatcgcttgaa
cctgggaggcagagtttgcagtgagccaagattgcgccactgcactccagcctgggcaac
aaagcgagagtctgtctctggaaaaaaaaacaaaaaacaaaaaaactatcctttaaactc
tttggttttcattttgcctctctgatctattgctcttcagcctttttttaacaggcttct
cttctgcttggttattaaaagtagaaatttcttgagtcttggtcctttggtattttctgc
ttctcttacttctgttatttctccccaggcagtcttaccactttgatttccatttatgaa
ggaactgcatacctgttgttccagcacttaatttgcctgagtttcagacctatttaataa
tgccatttagcttatcctagaggtaccttaaaaaaacgagttcaaatctgaactcataat
aatccccatccatgtgatttgaccctaatctacctccttagccttactattctttgttac
tctttgccatagctgcactatagcaaagcacggcctcttggtccaggcgtaatatacctt
ttcctccagagtctttgctcttatttcctatgttaatatgctcctcatttttcatttaca
acattacccagttttcaatacatacaaaagataaaaacattagttattagggaaatgcag
acttaaaccataaaataatcactaaaaccaactagaaggggtaaaattaaaaagactgtc
aactacaaatattctaggaagccatccagaacactcgtaaattgctggtgtgagtataaa
atgggagagccactttggaataccactggcaatttcttataaatttaagcatacaccttt
ggatccagcaattctacttctaggtgttttcggaaaaggaatgaaaacatgtccacaaaa
aacttttaaaaattatatttacagtcgatttactccttacagctgacaactagaagcagc
ccaaatgtcaataggattaatgactaaaaattttttggtatattcatacaatggaatgcc
actcagtaataaaagaactacagaatggcaaatgtacacaacatgacatggatgaatctg
acatacttggcaaaggaagctggacatgatactgtattattccatttatattaagttcta
gaagaggcaaagctactctggagtgaaaaaataaaaacagaacaaaggttgccttttgct
tctggaaaatggtgatattcaactgggagggtgcataaggggccttcttgaagtgatgaa
aatgttccatatctcatagaggtgtgtgttacatgggcttatctattaaagttgtacagc
taagaagtatgtatttcaatggcatacatgtatcttagataaaataaacaagttttatct
aaaaattatagaaataattgagttaggtgacagggagtgggaggtgtagatgaaagaaga
attgcagaacgttggtggttgctgaagttgagtaatggatgtggggaagtttagtatact
attttgtttgttttttgtgtgttcaaagttttccatataaagatctttttaaatcaaagt
tacgaagacacttaaacagcacctgtggtctgactgccagcttaataaagaatgcttact
aatgtaattttcttctctgtgaatcgtaccctgattcagcactcctttctccccaccagc
cactatcctgaattcctcttgactttttaagacatcttccaaatatcattctaaacgtca
cgtccatgggggaaagccttttctgattcagaacactcactagttttaagtgcccctgtt
aagtcctttcatagcttttatcttaattatgatttatttgcttaactattatcaacctct
acactagactaagcctaagtttttggagggaaagatatatctattcttgctgttttaatg
tctagaataataatacatttattgaatgattatgttggtgtagttttttaaaaaaattga
aggatacccaaattgaagagctattctattactgcttatgttgtaaggtcagtatgttat
aaaatgtatgaatgtgagagaatgactgagaaatgataatggtttctttctgattttatt
ggcatctgataaaaacaaggggcaataaaactgtgttattagatctggaactatatccgg
aaagtcctgtcagttggttctcaaatgcccttgagctttattatgcttcttccttattaa
tgtattaagccgtgtgagtgaaagacccctttcctctttacaaagaatattctaaatcac
ctcttttttctctcttctctaatgtgcttcttcctttgactattagttttggtatttatc
agggacagcgatctttaagagcttgttgttttgttattttttgtactgcttgctgtatgt
tttaaattttgatatatcttactgccaaatgaaggtgtgagctcataagtaataaagata
gaaaaaatctgctttgcttataattataccattaaacatgttgtccttaatgcagataca
cactattggtattgtaggtgcaggattatgttttgatttttgccagttcatatttccatg
gagtatttaggcaattttaacaacatagatttctgtaatgaatgtttttttaacttatgc
tgctgcttgagagacagagtcagactgccagggtttgagtcctatgcccaatatttccta
gctctgtgacttttgagttatctgctttttgcctgtttcttcctcattaaaacagagata
ctaagtgtaccttatagtgttgttgggagggctatttgaggtagtatatgtaatgcctgg
tacttattaaatgttagatcttttttactttcttgggttctagggagtccaataagtaac
aaatttttcctctaaggtttttttctttattggtggcaaaatatacatagtataaaattt
acatttcaaccatttttaaatatacaattcagtggcatcaagtacattcacaatattttg
caaccatccatctctgtccatttccagaagtttttcatcatcgcaaataaaaacttgata
cccattaaacaataactcccatctttcctcccctgatttcctggtaacgtatgttatact
ctgtctttatgattatgactattctaggtacctcataaatagaatcatacagtgtttgtc
ttattgtcctctcatttcatttggcataatattttcaaagttaattcatgttgtagcgac
ttctagttccattttttgtggttgaagaaagtacttccctaatagaaatgaaaacatgtt
tgcaaggctttttttaaagaatgcttatggatttagtcataataggtaacaactgaaaac
attggtatggctgggtcaagttggactaattcactgtagcccagggtcatgtaagaacat
ggagttttcaaggtaggcatgggggtggtattgaggggagggtagtaagcataaaagaca
gtgctcagaagaagataagggtgccaggtttcagttccagggatgttcacagtaaggatt
acagtgggctaaattttaagattaattttataaattctaaacacatcctgttgactttaa
ctgcaactcttaaaatgtatcaataacatgactttataattcctggaaactgatattttc
ccaatactttgaagaggtttaaaaatagttgactaatattagaaccaatatttcatgtat
tgatttttctctttaagtgacagttagtttagaaaatgctaaacttaagttagcagtata
gagtaggtatatagttatactgagtatgaatgtcttggtaggcttggaagcctaccattt
atgtcctcttgaggtacctgaattaccaaaagctttatgtattctgaagttattgaaaat
aagagcttttgggaattcaggtagttcaggagtgacttttctaaaaaacagaactgagca
ccatacactacttcttatagccttttaattactgccaattgcctagaataaagttcaaat
tcgagggcaacacaaacatggccctttgtgaactgacagacatttcctaataattcctta
catacatgatacccttgagccttacagaattaagaatttctgaatttaccttggtgtttg
atgcatctgtgtttctatacgtgctgtttatttacctaaaatagactattctctatttag
cctcaaaagtctcatggaaggttttaaaaaatcctgtctaagctctgttaattatattct
ttatactattatatttagtagttaatattataaactaccttacagtaatttttacagata
cctggcttactccctaaattgagttgcttgagggcaggaattgtgtattaatctttgctg
tattttctttgccttgcttactgtagttgattggtgctaattgaataaatgaacatttat
gcttttctttttgagaatattatatgggtagttcctttagttgtttaatagaagatagca
cagacttctatacagattggtttttatgggaaaacacaagagaataattaattaaaacat
aatttcatctagaaatttctgcagttggagcgaatattttttaagttattaacttaccta
gagtggaagattgaatttttgaatgatagtgttttagttattaaagtaaatggcatgtaa
gagaacttttaagttcatgtggcaaaacagggaaaaattgcaaagttagagtctgtaagg
tttctgatgtttcatagactaacaaaaggttgctttattagcagcattaatgaacgaatc
gctttattggcctaggcatactggtagcattgtcgttccaaagagctgagagttcttatt
tcaattcatgattatgttagacaagtgaagtgaaagccgttgaccttctcttaaggagaa
aatagaaagtttttacaaatagattatagattaaagccttttaaggaacatgatgaagaa
ctaaattactgctaaatcacttgaaaaatttatatttttttatgcaccagtcttataaca
gtaggttttttatagacctattaattgatatttaaaataataactttaataggatgattt
tgtagagttagtcaaagtgattgacaaaagcaaattattaaaatagtcaaactttcaatt
ctccaaagaatgttcagttttaaaagcaatatgagatacaggaagtattaaactggagat
gtaatgccacttggatggcatacttttaacattattttttatttgtttaaatggaatgca
ttttgcttattagaatcattttggaagtgccaatttgcttctgttaatttacattaaaga
ttaaaataggaaccactggtgaccttctaaatcaaagttcttcaaaacggttgttttaaa
cagcaagaattacagaactaagtattgtatagctattaaaactgaaaattatgtagcagc
agtccattttaaatttaacaattttaaatgatacttcaaaatataaaaaatacaaggaag
aatataagatgcttatgtatctgtactgccaaaatttaacaaatgttaactttttgctat
cactgtttcagaactttttaaaataaattaaaaatataattgaagattactaatctcatt
cttttgcctctcttctcagaggtaattacttttctaaaggtatcctgtaagttttcaata
ctttttaccacatatttgtataaaaagataaacagtactgatttccttttaaaatgtgaa
taataatatactgttggcataactttttaacttgcttttttcactcagtgtttatttaca
tcaagatttattcatgttgatatttgtatgtacttaaagtttgtttattttatcctctga
attattagtttggtccattatttatgatgtttagtttgattacttggttaaggtggtgtg
acctgatttctctattgtaaaagtatccttttccccttgataattaatcattaatctgtg
gggtaatgccttgagactgagtaaacattgtttctcaacagcctttcacccagtgttttt
aacatccattattgatttatgcctgaattattggttacattgatgcttgcaaaatgattt
tctacatctattattctttccacatttattagctggcattcttctttctgtaaaggagtt
tctgtcctttcactaagggctcatggtttcttcattaattcggtgaactaaaatccatta
ttgttatgtagtcatgcactgcatatacgacattgatcccatgagattataatggagctg
aaaaatttctatcgcatagtgacgttgtagctgttataacatgttaggactaacgtcaca
accaacatattacatgtttgtagtgatgttagtgtaagtcaaatctactgtgctgccagt
ctaaaagtatagcacatataattgtgtacagtacataatacttgataataataataaaca
gctatgttaacggtttatgtatttactatgctatactttttgttgttattttgaatgtac
ttctacttattaaaaaaaaaagttaactgcaaaacagcctcaggcagttccttcaggacg
tactccagagacattgttattataggagatgacagctctgtgcatgttattgtccctgaa
gaccttccagtgagacagtatgtggaggtggaagacagtgatactgatgatcctgaccct
gtgtagggctagtctaatgtgtttgtttgtgtcttaggttttagcaaaacagttttaaaa
gtaaaacaggaattttttaaaatagaaaaggcttgtaaaataaagataaaaggaaagaaa
atatttttgtgcagctgtacaatatgttagttttacgctaaatgttattacaaaagtcag
aaaagtttaaaaactaaaaagtttatatgagctaaggtttattattgaagaaagaaaatg
tttaaaaatagatgtagtgtggtttgagtgtatatttacaaagcctacagccgtgtgcag
taacgacctaggccttcacattcactcttcactcaatcattaactcacccagagcagctt
ccagtcctgtaagctccattcatagtaagtgccctatataggcatatgattttaaaaaat
cttttatactgtatttttactgtaccttttctatgtctagatattctcagacgcaccatg
ttacagttaccaggcagtattcagtacagtaatatggtatacaggtttgtagcctaggag
caatgggctataccgtgtaggcttgtgtaaatacactctgtgatgttagcacagtgaggg
aatagcctaacaaacacatttctcagaagatatccctgtggttatgtgacgcatgactgt
gtatcctttggtgactcaaattgttaaaatttagccagcgagaactcaatcagtctggtt
tctgtttctttgacatgccccctcatttttatagcaccagtttgctttgtggcacaacaa
aacattctctccttaccttatggttttcttaccccagacctggacttggctattttacca
aaagttcttgtttctcttagtggggaatggtatttggaagccaacatcatggatgcatta
ctgggtgtcattgcttctattggttcttttcattagacagaataagaaaatatactttta
aaaactagtgatatttgaattcctagtgacaccaaggttcttttctttccttgtttcata
ttcctcttccataatgaagatccagtttaccaataacatcaatatatgtttattcatttg
ctctgtcttatacatgatataacattggaattactatagcaataccactactaataataa
tctcaataaagtttaagatttctatgcagttttttatttatagcgtatagcccagtgaag
gtatatagtcagatcgctatgcttatagttattttaattatatttttcacataggcaatt
tgaaactttttctaataggaaggttaagcctatttacttatcttgacataactagtatgt
ttaatctaactgatatgttgtatttactgttgttttatgttatatatgtttttcttttac
tcttttttaaaaagttatttttactaatacaaaaatcttcctttctctagatatttagaa
tgttggtcttagctcttatagtttcccagtctttttggtatttttgttgtcattactatg
tttaattttgttaactagaattcatgttttcttttcttcatccatttaattgggctttat
actttttttttttttttttgagacagagtctcgctctgtcacccacactagagtgcagtg
gcgcgatctcagctcactgcagcttctgtctcccaggttcaagtgattctcctgcctcag
cttcccaagtagctgggattacaggtgcttgccaccatggctggttaatttttgtatttt
tagtagagacagggtttcgccatgttggccaggctggtctcgaactcctgacctcaggtg
atccacccaccttggcctcccaaagtgctgggattacaggcatgagctaccgtacccatt
ctatatattcctttgcataaatgttgtgttctgaggaaaatttgtgtctgagcatttaat
aatttaaaaaattaccatgtgagtggatttattttatatgaataatgtgccacatgtaag
ttgttctgcagttgattttccctttttgtggagcttttacaatattgttagtatgtttac
ttgtccctttttccccttgtatgtgtagccaagtaattataaaaaatggtgcactttgcc
atatctgagcttctctttttcccctcaagtagatactgtgcatttgaactgcaaaaaggg
tcatattcttgctttatctataatctcttttaaaggcaaaggctgatggttactttttct
ttaataagaatcttgatttttcattttgtatatttgatatttatcattttaagaaaaaaa
aatcctctatacatagttgttagaaatttatgtagaatggatgttggaacccaatcataa
ggctgtaaaaccataaaataagtagtaataaattttaaaaaatgaataggatttgtatga
agaaaataacaacacttcattctaggatcaaacaaatgaagagagagctatttttcagaa
tgaaaaaccaaatagtgttgcatgtagactctattcaaagttctagtctgtaaaattaat
agcaattctagtcaaaatagctgttttatcttttgagaagttcgggaagtttgacaatct
gattgtcaaatatatgttgtagagtatgttaatgaaactatccagtgtaaaatttaaaaa
taaagaccagtattgttatattgtactgtattgcttgctatactgtagtggtgtagtaat
taaaacaccattctagtgatacaggaatagggtccaaataaataaaaactgagaaggaaa
ttgtatacacatgcaagtgtgtgtgtgcacacacacacacaacacagaaagggttttttt
ttgttttttgtttttgttttttggagacagggtctcactttgttgcccaggctggagtgc
agtggcatgatcatggctcattgcagccttgacctcccaggctcaagcaatcttcccacc
ttagccttgtgagtagctgggactatgtgtgccactgtttccaactaatttttgtacttt
ttgtagagactgggtttttcatgttccctaggctggtcttggactcctgggctcaagtga
tccttcacctcagcctcccaaagagctgggattacaggtgttagccaccacgcccagcct
caaaaagctttttttttttttccctataaagagccatatatctaaaaagggaaacagatt
tgactctattacaatggaattctgtgagagaaaacattttaacaagatcagactatctag
tagaaaaatatttgtaacatatagcaaagttttaacaaacataataaagaccttctaaaa
attaataaaattgaaaaaatgggttatgaataagtacaaataattcaaagagtgtacaca
aattagttttcatagaaatatgcataaagatatatgtacaagtatgttaattttagcatt
atttataataccaaataataatattccaacaaaatagttggaataatctgaatgttcacc
agtagggaaatggtacatctgtattttagaatattagcaagctgattaaaaagaataaga
tctttgtgaccaggtatggcaaaatatcccacattattcatgaaactatagaatgatctg
tgtagtatgatctcatttacttagctcagtaggtctattgtttgtggtaaacttaaaatt
attttatttatcttataggatagggacttttaagttaacctattaatattattaggaact
aaaatatatcactgtaagagaataaagacaaatatagaataaaaggaactaagaaaaata
ttttacatcagattaattggaaatattggtatgaatccatgagtaaataaagaaatatct
ctccccccaccctgttctgtctaccagaaaatataagaagcatggacaatctcagtagca
atggttacatgcatcttggttgctaaatatggctccccattgaaagaattcaagatgtct
tagaaaaatagatgatcccagttcttaggaaaacacacagcaagccagaaaacaaggaag
tggtgaaaaactgaggaattatgtaaaaaagaaccaactcaaaggagttctagtggccac
ctccgaaataatgattgcaatcacaggcatatcttggagatgttgtggattcggttccag
accatcacaataaaacaaatattacaataaagtgagtcacacacatttttgttttcctag
tgcatataagttatgtttatacaatattgtagtctgttaagtgtgtaataacatgtttaa
aaaatgtacatgccttagtttagaaatactttattgctaaaaattgctaataatgatcaa
ctgagtttttagtgagttgttctaatctttttcctgctggagggtcttgactcgatgttg
atgactgctcgccagtcagggtgatagttgctgaaggttggggtggctgtgacaattttt
aaaaataagacagcaatgaagtttgttacatcaattattctttgatgaaatatttctctg
tagaatgtgatgttgtttaatagtgttttacccacagtagaaattcttcaaaattggagg
gcagttctctcaaaccctgctgctgctttgtcaactaagtatgtaatattgtaaatcctt
tgttattatttcaatgatgttcacagcatcttcaccaaaagtggattccatctaaagaaa
tcactttctttgctcttttttataagaagcaacacctcagccattaaaattttattttga
gatttcagcaattcaatcatgttatcaggctctgcttctagttctcttgctatttctatc
acatctgcagtgatttcctccactgaagtcttgagctcctcacatccatgagggttggga
tcaacttcttccagagttctgttaatgctggtgttttgatctccactcatgaataatgaa
tgtttttgaaggcgtccagattgatgaatccttcccagaaggctttcaatttactttgcc
cacatctatcagaggaattcctatctatgacagctatacctttatgaaatatatttctta
aataataagatttgaaagtcaaaagggcttctcaatccatggcctgctgaatggatgtgg
tggtagcaggcatgaaaacaacatgagtcttctggtatatctccatcagagctcttgggt
gaccaggtgcaatgagcaatgtcagtgagcaatgagtagtattttgaacggaatctttta
ttctgagctacagtctcaacagtggacttaaaatattcggcaaaccatgttgttaaatag
atgtgctttcatcaaggctctgttctgtttgtactgcacaagccagagtagatttagctt
atttcttaagggccctacaatttttggaatgataagtgatcattgttccacttggaagtc
accagtagcattggctcctaacatgctgagtcagcctatcctttgaagtttggaagccag
gtattgacttatcgtttctagctaggaaagtactacatggcttcttcttccagtagagag
ttatttcatctcaattgaaaatctgttgtttagtgtagccaccttcatcagtgatcttag
ttagatcttctagataacttgctgtaacttctccatcagcacttgctgcttcatcttgta
cttttatggagacagcttctttccttaaacctcatgaaataacctctgctagcttcaaac
ttttcttctgaagcttcttcacctctctcagccttcatagaattgaagagaggactttgc
actgaattaggctttggcttaagggaatattgtggctggtttgatcttctgtgcagaaca
ccaaacctttgtccatattagcagtaaggctctttcttgttcctattattcatgtgttca
ctggagtagcacatttaatttccttcaagaacttttcttttccttttttaacttggttaa
ctggcacaagaatcctagcttttggcctatcttggccttcatcattccttcctcactaag
cttaattatttccaacttttgatttaaagtgagtaatgtgtgactcttcttttcactcca
acacttagaggccattgtggggttattaattggacaaatttcaatgttgttttgtcttag
ggaatagggaggtctgaggagagggagagagatagggattggctggtggttggagcagtc
agaacacatgcaacatttatcagttaagcttgccatcttctgtgtttgtgatttgtggtg
gcccaaagcagttaaaatagtaacatcaaagatcacagatcaccataacagatacaataa
taatgataaagtttgaaatattgtgagaattactaaaatgtgatactgagacacaaagtg
agcacatgctatttgaaaaattgcaccaaatagacttgcccaatgcagggttgcacaaac
cattattttgtaaaatccacaatatgtttgaagtccaataaaatgaaatgcagtacaagg
aggtatacctataattgtaaaacctttgattgaaaaaaataatccatgagtccagagaga
taagaagaaaaggaaggccctcctttagagaattgtagtagcagtgttagaactggaaaa
ctgtactgtaaactccaaagaaacaatctgtgccaacagcatcaatggatgccaaaacct
ttaggtgtaagagtgctgtgaacatgatacgtatgcagtggtgcccaattattctgtaaa
tcttgtacgactacaaagggaaaatgtgcctttgcaatagatctatagattgccacttga
actaagtggtcacacttataatgggacagcctgataccatgttccttttgttatgatata
atgcggaatgatgtaaccttgggtgtattcttgcctaatgtttatctacaatccagtcag
gtttctagacttgatcttgtttccaagaaatacaggaagttgtggagtaagtcagaatac
attctgggaagcagtcagacaaatctaggatgtgggacattctgtaagaaaactggccat
gagtcctgacacatcattgtcttaagaaaaagatatgtgtgtgtgcgagagactattcta
ggacaggcgagaccaaagagatttaacaggcaaatgttatacataaattttcatttgccc
ttggattgctctctcaaccctgaaggcttaaaaatagatatctttgagacacttgagaaa
acatcaagatactagcttaccttatttttacttttttttgactagatgtcttaatggttt
tattactttgtaagacttgttttaaaagatatgtttcactactcagtatagtctatgata
aaatcagtaaatataaaatatttgtcatttgcatctcttaggaggttcgtctccattgat
gaaagctcagcttatgtgtaactgtcttccaaagtagcttgcttggaaggacattcacta
tcatcatattctgctttatttcatagctgtgtaatcacatctactacatccttgtttatc
gtttattagattactgcttttaagcctattttagttctttaatctgagaataacagttga
ctcagttaatttcattaaagcaagattgcctgtaatttttttataaccttagaaaagtac
tatgcccaacagcaaaacaaaaacctaattcaaaaatgggtaaaggacttgaacagatat
ttctccaaagaagatataaaatggccaataaacacatatgaagagtctcaacaccagtag
tcattagagaaatgcaaatcaaaaccacatttagatacaatttggctattagcaaagaca
cagaaaatagcaaggattgggaaggatgtggagaaaattggaaccatgcctgcactactg
gtgggaacgtaaaatggtacagctgctatgaaagacagtttaccagttcttcaaaaagtt
tgatgtagaatgattgtatcatccaacaatttcactgttaggcttatctccaaaagaatt
gaaagaagggacacaaactggataacttatacagcagtgttcatagcagcattactcata
ctaaataaaagatgaaaaaggccaaatgtctgtgaacagatgaatggacaaatgtgttta
tacatatagtagaatattattcagccataaatacaaatgaaattttggtacatggtacaa
catagataaaccttgaaaacattgtgctaagtgaaataagctagacagaaaatattgtat
gattctacttagatgagataccccaagctggcaaatatataaagctggcaagtataatag
aagttactaaggcctagggtgagggaggaatgagaagtttgcttaatgagtatagagttt
ctatttggaatgatgaaaagttctggaaatgaatagtggtgataattgtacaacattctg
aatgtactcaatgctactgaattgtccatttaaaaatggctaaaatagtacattttatgt
atattttatgatacaaaaatatttttaaagtaaaagtttgtattttgatgttacttgtga
aaaatattttcactaataaaattttgttgtttcattgctgcctctttgaaaatgtattaa
aaattatatgcattttgaaatattacagctatataaatgaaaaagagtatcagtaaagaa
ttttagctcaatactctcatatcatgttatttattgtcctttctgtttcacattaagata
tacagagtcaacaatttaaatttgcagattaaaaaacactttctgcttttggccctatag
aaataaaccaaactggaaaaggtgtcatgtttcctagattgtggacacagaaaaagaaga
gctcaaaaggacaagtaactcagcacagtagatggttattgacctgatcatatttaagaa
tatcctaaaaatgcaaaaatgttaagaagcaaaactgggaaatactgggatgatatatga
aaaattcaaatgacaaatatgcagtggttgagtatttatgggtttttttggggtttacta
ttttttcccccaaatttaatgtctcctcttttaaatacttctaccggattctctttagat
gatgctgcaattggattgagacattttcattgtttggctttgtaagatttccaaatgcgt
atagttgttaaaatacgtattcaatgagggtggaaggaaacacacaaaaatagaagtatg
tcactctggtaagagatgataggtccgatgactgctaatacttttccaaatggccttatt
tggatccttcttagaacttttatatttttctaatattgacctacccctttggaactagag
tcccattgtcaagaactgtgaagaatcttgacattttaccctacttgcaagctagcagtt
actcagttgtagttttatggatggtggttaaagatatgagactcctgagttggagttgaa
ggacagtttattagtgcagcaagcagtagcagtgaccagagtattagcttcctttttttt
tttttgccgcttttccaagccctggttcccatatagtagtgtgaagaggagggctacatg
atgcttgtacacactgtggattgcataacagatgcggaatcctcagcttcttacagaatc
tgaatagtagtgtataataggaagtaagaatgcctttctttgctttggagcaagatactg
tctttgtctcccaacactattgctgtaaaagtatcctcaaaaggatagtgcagaccaaaa
gggtagttgagtgtcttgctcctaagaggtgcagaaacatgagagtctcatagaaaattg
tctcccaataatattttctgctcatttctatactattttgtcttaaggcaaatttttttc
atgagtataccactctgatttatctgacagaagatagtgaagtatctgttcaaatagtta
gctggcttttaaaatcaggttatttgctttcttattgttgagtttgagagttctttcttg
attctgggtacagtcctttgtcagatgtgtgatttgcaagtattttctctgagtctgtga
cctgtcttttcatattcttcaatctcttttacagaacagaagtttctaattttgattact
atcaggtttttctcttatgaattgtcttaggaactttttgcctagttgaaaatcacagag
tttttttctttgttttgtccagatgttttattattttgtgtcttacatttagatctgtga
tctgtcttgcattaccttttgtttgcataagttatgaggtagagttcaggttcattgttt
ttgcgtatggatatttacttgttctagcactatttgttgaagagactatcttttccttcg
gtgaactgcctttttacctttgtcaaaaatcaattgcccttatttgtaagtatattttgc
tggatttattattctgttttgttgatttctgtgtgtgtcctcttgctaatacatcattgc
cttaaatagtaacagttttatggtagttattgaaattagtgttctcaaattttgttcttt
tgctgaattgttttggatattatagtacctttgcattttctttataaattaggaatcagc
ttgttgatagctacaaagatactttgagattttgattggtgttatgtttaatgtacatgt
cagtctgaggagactgacattttgccaatattgagccttccaatccatgaatatagttat
ttttccattttttaggtctttgattattttcatcagtgctttatagttttcagcatgcag
atcctgcacatattttgtggaagtgtatcttttccagatgctattgtgagtgctactgct
ttaaaaatttttgatgtactaacttattgctgatatataaaaatatggtcgatttttaca
tattgattttatattccacaacttgttaaaatcataatcataaaaccctttggattttct
atatagacaacatgcttttgaataaaggctgttttacaaaatttcattttaaatttgtat
accttttatttcatttcctttcttaattgcacttgctagagctttccaggacaatgctga
atagaggtggtaaaaacacatctttgctttgttcctggtgttttattactcttttattat
tagtgttatgtttagagatggggtcttgctcagttgcgaaggctggagtgcaccggtatg
atcaaagctcactgcagcttcaaactgttgggcccaagtggtcttcctgccccgccttcc
tgagtagctgggattacaggcatgtgccatcatgcctggctaagttttttattttttgtg
gagatgaggtcttgctgtgttcacaggctggtcttgaactccagtctcaagtgatcctcc
cacttgtactgggattacaggcattagccaccacacttggcctcttattatcattattat
tattaatatatataaataaatatacatatatttatttattgagacagagtcttgctctgt
cagccaggatggagtgcagtgccacaatctctgctcactcaacctctgcttcctgggttc
aagtgattctcccacttcagcctcctgagtagctgggattactggcgtttgctaccatgc
cgggcgtggtggctcacgcctgtaataccagcactttgggaggccaagacgggcggatca
tgaggtcgggagatcgagaccatcctggctaacatggtgaaaccccgtctctactgaaaa
tacaaaaattagctgggagtggtggcgtgcgcctgtagtcccagctacttgagaggctga
ggcaggagaatcacttgaacctgggaggtggaggttgcagtgagccaagattatactatt
gcactccagcctggcaacagtgagactccatcttaaaaaaatgttatgggatgaaatttt
aaaaagacctttgaaagatacaagataataggattcaattctgtataaggtagagataaa
ctgttgatttgtactagtttgatttgtgaaatattacaattaggctaaatcaaataggtg
atgggtaatttgagaattgatttctaatgctttgtcctagatcattcaaaatctgatgtt
cacataactacttagaactcttgactagttgtctgtgctttttaaaaacctttacctggc
tggctacaacttttctaccgccataattaaaattgaaaacaaaataattagtctcttagg
aaaataatttacgaacactatctaaacatgctagatttaaaactaccagtatttacatgg
ttagaattcatgagcagtcttcattagacttactgctgaatgaccagcatacagtcgctg
aattcattgtgcatcttttaagggaagattgttttgcttgtatctttgcagtcttctctg
agatctttaatgttactagaagttataaagtttggttaattttcctaataagcctttatc
tgattctgtgggctatttaggtttgggatgggatctgttgtcttaatttggtcaccattg
gctcttggcttcaaagtggaccctaaactatattcctagtctgtctagtatttgtctttt
ttttgctgagatatgtgacccctggggaggtttcccttattttcaataagttaatctttc
tcttttaggaagaaaagacaaatttctcataagggagggattttttccttttttgttcta
gagtgcctttcaaattaacaattttgttcatatcaaaagttgcccataatgatcactgta
agtctcaagttattcttggtgaaaatatgccatttagaaagataagtatctgaattggaa
gaagcatgtttcatgcttggagaggttgtggagttagactgacttaaaaagtcagttttg
tgcagcctgggcaacataacaagacccagtttctatggaaaataaaaaaaaaattaggca
ggcgtggtggtgcacacctgtagtcctagctactccagaagctgaggtggattgcttgag
cccaggagtttgaggctgtggtgagctatggtaatgccactgcacttcagcctgggcaaa
agagtaagacctcatcttttaaaatatatatacaggtttttcttcaaaagattccaaagg
tactgctttaaaaaatgtaagattttctaagcaacagggccgagtactttgctttaaagt
ttttatgaatgcctggcttggctatctggttgcttttgaaatcaacatcttactaaacta
ttaatgcttttaaaatgtgtgcagaattacatatttgtaaatttaacttgagtctgtcta
ggaaagcattagcaagacttttgtttttgtttttgtttttggagacagtcttgctctgtc
gcccagcctggagtgcagtgcaggtcactgctcactgtagccttcacctcctgggctcat
gctatcttcccacctcagcctcctgagtagctgggactacaggtgtacatcaccacaccc
agctaagtaaaaagatgtatttttcatagagacggggtctccctatgttgccaggctggg
cattagcaagattttacgggaaattttttttatcattagtagaaaataataaggaatccc
ttgctggtaaaatttatattgaaactactcttaaacatatttatataaaatgaccagaga
tatttaatttacctttatataataaatagaaataaattgaaatacattattaggagattc
agattttttaattagtaaattagtgtgatggcttaaagttattgagaggtaatttttcat
ctttgattagaaaaacttaaaaattgtataatttacatacacaaagtatatatgagcttt
ttatattgatgaactttacaaaatgaacacacttttgtaaccacaacccagatcaagata
tcagcttctcaggagcttccctgtatctgtcatattcctttacatgaaaggtaattaata
ttctgactttgtaattagagattagcttggcttttttttgaatttttataaatcataaag
tatataagactatatgatttataagttttttcaaactggcttctttcccacagcagtatg
tttttgagatggatatttggcttacagcatttttcattttcactgaggcttagggtttca
ttgtgtgaatatccttagtttatttctcccttctgctgatggacattcaaattatatcaa
ttttgggttcttacaaatagtgctgctgtgataattctcgtacatatttttttgtgcata
tatgtacacatttccattgagtgtatgctcaggggtagtatcacttagtctttgggttta
tcttcagcttagtagacacttccgaacagtttcccatactgtttgtattatattattata
catttttacctgcagtattttagaatgtcagttgctacagatttttgcttacacttggta
ttgtctttttttagataaaagttagtcattttggagggtagtagtatgtctttgtgaagt
tttgtttggtctttagcctatcttaaaaatcggtttgttttttttgttgagttgaagtat
ttttaaagaatatattttgggtatgagtctttagtggaacatgtgcattgctgatatttt
ctcctacactgtagcttgcattttcatttttaatttatttaaattttattttatttttta
agagacagggtctcactctgtcacccaggctggagtgcagtggccttatcatagcttatt
gcggccttgaactcttgggctcaagcagtcctcctgcttcagccttctgagtagctggga
ctgcaggtatgtgccaccacacctgggtaatttttaaatttttttgtagagacagaatct
cactatgttgcccacactaatctcggactcctggcctcaagcagtcctcctgcctgggtc
tcccaaaatgctgggattacaggtgtgagccactgcgccagtcatttccttctttccttc
tttctctctttccctctttccctctctctttctctcttcctttctctctccttccttcct
tccatccttccctccctccctccctccccattcccttccccttccctccccctttccctc
cctgcttccctccttccttccctccttccttctctctttccttccctccttccttccctt
tttccttccttccttccctccttccctccttccttctctccttccttccctccttccttc
cctccctgtaattgtaatgaaattgtttgtttgtaatgaaattcagtttttttatgtgct
gtgtattaaatctatgcctaaatatcgtgacagtatttatcttgtcactatataagcttt
attatttaaaattttatatatataggtctctaatatatcttattttttttcccacctttt
ctgtggcttgtggatagatgttattcattttactgattttgattttaacagtagtagtca
tagaaaatctattgatgtggcttactcatgagaaagcatttttatttttatatgtgatat
tataatttctagtatggcaacattttctgctagttatgatatgtgatttttactctgcag
attctttaagaggaatttaaaaacagctttattcctgactttagagaaggcagtcagttt
tacttaatatgtcattttatgtcttatttgttatgttttgtttttgaggagtcatactgg
tagtttaaaaatactgagttttatcatcaaatgcagctacaatgtttaagcagatgactt
aatctccgtgaggcatgaggtgattaaaaaatgtatgtatatgctttttcaaatacagag
taaattgcctggcagacaagaagagcttcataaatattatttagtgctttttattctaga
tttttgaaataaattggactcgacttgatgcaaaggatttgtactataagtttattataa
aattattaaccgctagcttttgtaaatagtaaaattgatttgcccattatacatttcttt
ttgttatgcataaaatattttaatttttaagtaccaacataggatggatttttaaatgct
tcctcatgttaattttttaataaacatagaatgaatactgtgtgaaatattttcccactt
tattggctctgttttaactcagtagactctatagttagattgtctaggtgccacttaaaa
gatggtaactagataccagaatgtgttacattatatgttgattgtcaggaagaactgaat
gcatgaatgcttccctaagacattgttagcatgccgtggtcttgatctataattttttgt
ctaagttgtgatagagctcattctttacatggtctagtaaattcagccaatgtctttata
attcattagcatctctatatattacctataaaatttaaattataaggctatgaaaaaatt
gtttcatatgtataatataccaaagttttagcatatggtttggattttaaactgttttac
tgaaacaaattaccgtcattaaatggaattcttaggtagacattacagagtaggtgtaac
tctgaagttgaaaatggtttcatcttcttttttaaaaaattttattatatagggaatctc
aaccaccagcaggattttaaaaaggctatctaaatatttggtaagtaaatattctccatg
ttaaatattttgcgtgttacttaagtttacacatatgctacatgaatgacatttacaata
gtgagtgtaaattatagttataattaatattttaaattttacttggttactagaattttt
gtctccttaaacctattccaatccttttctctcttgttctttatgttgtgtccagagatt
ctttcagtgtacagccttattattaagagttcaaggaaagaaatggttttagtgagcagg
aatgctaatactttatgaataaaacaggatttcccctacttgagattttgtagtatttct
tttttgtgaaaacactaatacagatttgttctttagccatccattgcttgactcctcctt
gtatttaggtcctgtttgctgcaacatgccaacatgaaccaaatactgtggtcagggtct
atgcatttcctgtttagattattctttgttccaagcttaaaatatgaaaaattttgtagg
tgtctgtagttaagaatttcataaacttatgatttcatagataaagcttttactttttta
tttttgagtaaaggtaggtgagtcaatctcagaatgtattttacttcaactacatagtat
tttatacctgtgtactatggtgattatatagatataaaatgatttggttaatcataatag
attttaaagttattaaatatgcttcagaatgggaaagaagtgctgagacattgcataatg
aggtgtcatagtataggcttttggttgtttttaatatagtcaatagcagattattaagga
ggaaatatttagtgataagcattggcttcttttattatactttttaatgtgtttgaaaca
gcaaatatttgaccaaattatttttcttaaattttatagtaactacagtaggtgcctaaa
tgttcaacagttatactgttctgtttactgtaaatttgtaataaatataaatacttttag
gagcaaatgatgccacagagatctcttaaacagatttttactactttagaaggtggtaac
tgatttctggagttgtattaagtacaaaagagtaacttattgtcatttttttccctattg
atgcgtatatttgagttagagggaagcagtcgtctttgtgttatatgcttatcatgtata
tttcatcttagcaaatttattaattttagtgacagttttattgagatgaaatttatatac
caggagatttcatctctaagtgtacaattcagtgatattttaatatattcaccaagttat
gcaactaccagtactttgtaatcttaggttactttcatcatccttcttctggacgccctc
tatcacccagccctaggcaaccactaatacactttctgtttgtataggtttgcctattct
gggcattttataaaaacagaatcataaaatatttgtccttttgtgtctggcttctttaat
ttagcatgatatttaaagcttcactcatgttgtagcatgtatcagtgtttcattcatttt
tgtggctgaatatttctttgtatatcatttgtgtattcatcagttaatgagttgtatcca
cttctttttttggctattaggagcgcttttctgtgaacattcttatataagttttgtgta
gatacgtttttgagtatatatggatggggtgggattgctcgataatatagtacctgtgtg
taactctttgaggaactctcaaaatggctgtaccattttacaataccaccaacaatgtat
gaggattgtaatttctccacgacctcataaacacttattgtcagtcttttgtatttcagc
cattctagtaggtatgaagttgtatcccattgtgattttgatttggataatgactaatga
taagcatcatttcacatgcttattgaccatatatcttctttggagaaatatctgttcaac
tcctttgcccattaaaaaaaaaaagattatctttttaatgttgaatattgtaaacattct
ttatatattctggaaactagtcccttatcacatacatgatttgcaaatattttctcctat
ggattatctctttacttttatgatggtgtcctttgaagcagaaaagttttaattttgaag
ttcagctgatctgttttttgttgcttgttcttttggtgttatattgcagatttgttttta
aataaatactcactgatagcctgggtctatgacaaagttttaatcgtcataggactacga
aaaagcattggcatttcttgtgtttgacagtcaaagcccaaaggaggcaattatccttgt
atttgtgcttcagttcctcagctaattgttttaagcaaaaccttacgttttgatcccatg
tgttactgtgaaaatattagctgtgtgtgatatgtttattgatattagtcttgtgaattt
atgtttagttttttttggattgaagaagggaggggccatccttaatagatgttattagtc
cgttctcacgctgctgataaagacatatacccaagactgggtagtttataaaggaaagag
gcttaattgactcacagttcctcagggctggagaggcctcaggaatcttacagttgtggc
agaagaggaaccaaacacatggtgacagcaaggagaagtgcagagtgaatcgggggaaaa
gccccttataaaaccatcagatcttgtgggaactcagtcactattatgagaacagcatgg
aggtcatcatccccatgattcgattaccttgcactgggtcccaccacaacacttggggat
tgtgagaactgcaattcaagatgagatttgggtttggacacagccaaaccatatcaatag
tttattatgacaattactgcatattttatttcatagtttaagaaaccattgctttttaga
ctaaggattatcaggttcttatctttcttgtgaagaaggaatagattttgataatctcac
tgattttaatcttgtacagattattattattattattttgagacggaatctcgctctttc
gctcaggccagagtgcagtggcgcgatctcagctcactgccagctctgcctcctgggttc
acgccattctcctgcctcagcctcccgagtagctgggactacaggtgcccgccaccgcgc
ctggctaattttttgtatttttagtagagatggcatgtcaccatgttagccaggatggtc
tcgatctcctgacctcgtgatccgcccacctcggcctcccaaagtgctgggattacaggt
gtgagccaccacgcccaacccagattatttttaagcaatgatattttggtgtttttgtaa
acttatgtatggaatatgtaagtttttcctctattaagtaaatgtctttaattttttttt
ttttttgaaatggagtctcgctctgttgcccaggctggagggcagtggcacaatcttggc
tcactgcaacctctgcctcccgggttcaagtgattctcatgcctgagcctcccgagtagc
tgcgattacaggcatgtgccaccaaacctgactaatttttgtatttttagaagagacggg
gtttcaccatgttggtcaggctggtctcaaactcctgacctcaggtgaccacctgcctct
gcctcccaaagtgctgggattacaggcttgagccactgcgcccagccagtgtctttaatt
tttaaggtaccaatttagttccttattttttatcgttatgtgacgttatgtgatcagttg
aaggtgtatttcatgtattttgacctataatgttgtctagtttagtttctggagaaatgg
tgtgtaacagtggaatgtcacttgtcttcaaagcattagctttctgtgtaaggttgaggt
tgtaatataaatgcgctagatgtgtttttattaggggaattgactattcttacctttcat
gttatttgacagtattctttgtcaacttcaaataacggcctattttgatgagattttata
gtgctaggatcactgatattcttgttagttgatttaaaatcatggtgttcattggctaac
agtcacttgagtggctattagatgggtaatattgttctcttttttttttttttttttgag
aaagagtctcactctgtcgcccaggctggagtgcagtggcatgatcttggctcactgcaa
cctctgcctcctgggttcaagcgattctcctgtgtcagcctcctgagtagctgggattat
aggcgcatgccatcatgcccggctaatttttgtatttttagtagagatggggtctcacca
tgttggacaggctggtctctaactcctgacctcaggtatccacccactttggcctcccaa
agtgctgggattgcaggcatgagccaccgcgcctggccagatgggtaatattgttctata
cattgatattcttatggtttatgttatagtattcatggaaatttagcaatggaaaaggaa
atgaaaaacttattaatgatgttgttaaattttcaattgtgatgacagtaaggaaaagac
ataattgagagctacctccaattgtttatatcaaatgtggtaattaagaggattttggtg
acctatttatggctgaattaaggcaaaatagcttttatatccgattctttcctcttccct
tctcttgttctgtgtgcttactatcataataattgagtatacattaattttctttaagta
tttatattttaaataaggttttgatgtcagtagagacttttttttttccttgaaggtaag
taccctttaaaccactaatttgtcactagagtctatagtcgcttccatatataataggta
gcacagcttctcaggtcatgtcctgctttgctgctctgctgaccaataatacaccttgga
gatactggttctggttatatgaatagaacaattaaattgttcttcattagatggatcttt
gattactaatcttactcagaaaggctcaactggacttggattattctaattgccaatact
gtttttgcacaaatgtctgcaaatcattgaacagtaatactgacccctttttcttccaat
aaagttattgcagtaatcacatcattgcagagaatatttcttaaaagcttttttcaatat
ttacttctttaatgaaaaacttaatccagattaagccacatacatagacgtcttgacttt
attattttgaatacctgagatttccaaaatctctttaactgtcttcacacaatttctagt
ctgtcattttaattaattacaactgttcatttcttctgcttttgtttcctcttcatgtgc
tgcctagcgttgctagagtttcatcagtttcctgccttgagaggtttctagtacaaatta
acattttcccttcctagtttatgcaggttccttcctggtaatttttatctttttgatctg
tttcattctgcatgttagttatttggaaccattctactgtgctcaaatgaacagtcctat
ccctagttctcttattcctttttcatttcatattttataccctttttcctcagtctgtct
taaagaataaagtgttttcatttcaaaagcttattatctctgtttttaatcttagccctt
ctttcctctactatttttcttcctcttaatgctccatgctgggttctctgccttatcaga
cttaatcttgaaaagtacgttacttccttcccattatctccaaggttgttatagtccatt
ctaggtgtctccaatttctcaactcttactcaattactttctgaagtttgactattattt
ctactagtgttcagctttcttaagtgtaagtaaacacatttcttacttaactaggtagat
aaacattagctggacggatctggccgtaaacatgctattatttccaagataaaactgagt
tccaaatggctaaatctttgtcttttcacttttcatccattcctgtcttttttagcattt
gtttttttgctgcttaacattcacttctcctttgtcttctgtaataaaccttaatggttt
ttctcctctctctggctaaatgtttgttttctttgttactgctttcttcttttatatacc
acttatatataaattgggttgaagctttgctcttagctctttttcattacacatcatttt
gaggagatggtctcgtgttcattatgtagattatcacaaatttgatgactcagaaatacc
acatttttcttagtgacctgttttaccactttctcagtcgccaggtccttagcaccccaa
aattgttatttcccagtggaaagtttgtcttgtacaatgcttgctatatggttggaacta
agtaaatgctttttgaatcatgaactaactccttatctgtctttttttttcttaagataa
tggttttaactgtccacctattctcatgtcacaagccctgtaatatctctgaccacttct
tgtctacacttgttaatattagttgtcatattttggtcctaagtttgactaatgaggaag
atgacatttttgtctgcagatggtggaaataaaaatcacagagattgtgatttccttatg
gttttgtgggtaatggtggagtttaaacttacaacgaagtttctggtgacatgtttccta
gttttcacagaaaacattctttttttttttgagacagaaagtctcactgtgtcgcccagg
ctggagtgcagtggtgtgatgtgtcggctcactgcaacctctgcctcctggattcaagtg
attctcatgcgtcagcctccaagtagctgggattacaggcgcccaccaccacgcccagct
aatttttgtatttttagtatagatggagtttcaccatgttgaccaggctggtcttgaact
cctggcctcaagtgacccacctgtctcagcctcccaaagtgctgggattacaggcataag
ccaccacacccagccaaaaacattctttataatgatacaagtaatataccagacaagaaa
ttactcagtgtactatgtgagattaaaaaaaaaaaaaaaaaaagactggaagtggaagtg
ggagtacttcaaattcttgtctgatgaggtaaagattttaacttggtggaaggaatagaa
atcaaggatgtgagaaatttttcattaattagaatttcgagattcttcttttaagaatgc
agtgatgctagtgaagtggttgagtgaaccatctttcagctaacaattataagatctgga
caaaatattgaaagaaaaactttaactgttttaaaagccatggaaagtaaccaaaaaagc
aaagccagatgagagcctccaactgcaatgtgtaattgtatgtttgtggctttcttgcct
tatggtgcattttcgcctcctgtctttcagtggcataggaaagaagttacgggtagcctg
gcagttggaaatttactcaggggaatcatggaagcaataagtaagatccaaaatctgagt
gtaaactctgtccaaatcatggctaatcacgaaactatacatgcataggggagaccccag
gggacccagtgtaaaaacaggcagaaacaagagcggaatctgctcctgaaagaattgaac
cctgtgagatttgtgattgcttctttttttgacagtacatttctcaacttgcatgcaatt
tgcgtggccaacagctggcttgaggtatcagagcacagaacatattgttgacagaagagt
taaaatttagggaacaaacactgaaagcaagataacatcagaggatgtaggccacacatc
tcagtataatctctgcccatttccttcactgaccaccagactacccaggtgcagagggga
tgccctgggcatcaggttagaagaaggaagcagctggaaggtgaaagaactaagcaaaat
cattgttgcttactataagggagacacacagtaagtcctcacttaacattattgataggt
tcttggaatctgactttaactgaaacaacgtataacagaacatttttttctcatcagttg
tataacaaagcagtgttaaaggaaacgacagtattcaaggaccttgctgtttgtgttttg
cttcaagttgctgtttccaagacctatcgatggtattaagtgaggacttactgaagtttg
cagtttgagtccatacgagcccattagaatcatacaacatgaaaaccaaatggtcctttg
aaagattgcgacaaaatctggttgctataacttgctatttacaatgtgtattttcaacta
aaaattattagatatgcaaataaagtggaaatatgactcattttcaggtaaagaaaatca
ttcaatagaaattaaccccaagtatagccagatattggatttagcaagcaaggactttaa
aatagccctaaatataatcagaagaatttaaggagtcctatgttcaaaggattaaagaaa
aatacagtcacccctctgtttccatgtttctgcatttgcagattcaaccaaccgtggatc
aaaaatagttgggaaaaaacataataaaatttacagtacagcaataaaaaataatgccaa
taaaaaacaataaagtcttgttcagtgaatagataggagatcaaaacagagaaacagata
ctgtaaagaagaagcaaaagagaattttagggttgagatcagtaactggaatgaaatttt
tatattatattttattttattttattttgtggtggagtcttgctccgtcgcccaggctgg
agtgcagtggtgcgatctcggctcaccacaacctccgcctcctgagtagctgggattaca
gacatgcaccaacatgtctggctaatttttgtatttttagtagagacagggtctcaccat
gttggccaggctggtcttgaactccctcccgacctcaggtgatctgcctaccttggcctc
ccaaagtactttgattacaggcgtgagccactgtgctcggccaagcaaaacagttgtttg
gagatggcaaaagaatcagttaaagatagattaataaaagtcatctatttgaaagagaaa
gaatggagaaaaataagcagagtttgagagacctttggggtaatacaatgtttgaacatg
catctaaatggagtctcagatggggaataaataatggctgaaaacccccacaactttggt
ggaaagtatcaatttacatatccaggaagctcagcaaacttcaagcaggataaacacaca
caaaaaaacagatgttagcacataagtggtcaaattgctcaataccagagaatttcttga
aagtagccagaaatatcttaccagaaacaatggcgacctctagccatggatatggtatat
tcagctgaaaggcaaaaagctatcaaataagaattctgtgttcagctagaagtgaaggta
aaataaatacatgttcagataaaccaaaactgggaaattttcaccagccagccaccattg
caagcatttctaaaggaatcaatttcaagggaaatggcaccagatggcaagtaccatcta
caggaggaaatcaacatcaaaactagtaaatacataatttactctaatttttataaacta
tatttttccttattttaaaaacaagtacctgtgacttgaagcaaaaattatgatgctgta
ttgttgggtttataatggagtttgggaatttacatattgtacaatttttaatatgtaatg
acatacaaattaacgtgttaatagtcttctaacttatgaacatggtatatctcattaagg
ttattgtttaattttagtaatgtataataggtttctaaaaaacttaaaaatgtggtaaaa
tatgtacaaaataaaatttgccattttagctgtacaatttggtggcacagttgcattcac
agtgttgttcgttcattactactttttccagaacttttctatcattccaaacagaaactc
tgtacctactaagcagtaatttcccatttcccttccccgagtctctggcagtgtctaatc
tactttctgtctttatgaatttggctattctgtatttcatatatagtagtcccccttatc
cacagttttgctttccacaattttggttatctgtggtcatccatggtctgaaaataggtg
attatagcacaataagatgttttgagagaccacattcatataactttattacagtataat
tgttcttttttattattgttgttgttaatcttttactttgcccaatttacagattaaact
aataagtatatatgtataggaaaagacatagtatatatagagtttggttctatctgtggt
ttcaagcatccactggggggtctggaacatatgctctgtggataagtgggggactagtgt
acttcctgccctaccccttggtttggagtcgctgctgtatgaatataagggcagtctgcc
tgtgatgtgactctccattgatcacctgggtgtatgaattaattgctgtctataaagagt
cttggaatgactattgagtttgctctgtgtatgtatacatacaacagtatggctactgtt
gtagctgatttggctatgatactgtagtatcttatttgctccaaagagatggagtagtga
tgaaatctggtttactaggtggaagctagtgtaaaaatgggtactctgttttattgaatt
agcttgattggaaaaagtgaagtgctgatggttttgaaaatatgatgataatgatgtcat
ccttctggtttaaatattttgtagcacttgtggtagattgaatgctggtgtcggtagtaa
agtcatgctgcagttatagtctgaaccagctgtactgttttgggtagtaacttagacagt
agagaacaccacttttctaggcagggctcctcacctctcctagggggccatttcactgca
tcttggagtgaatatacagagaggaagtagctagatcctaatttctacaagttatatcag
ttggggaacagttgtggttgtcaacctgtgttagggctgtattggttttgctttcagttg
tgctataaagtggaaaaaattgaattgtttatttatctcttattgtgaaactttcctgtg
gcccatagaagtggcatagcaagctgaacatagctgggtttgttcttcattgcaagatcc
tacccccacctccctttctttcctgcctggtttcttgtgtccatactgcaagctcttttt
cccagttagaagtaagttagttcactcttaatagagctgatttacaaaggaattttatgt
gtgtcatactgtttgattctctgaaaggtagatatgggactgtggtaacaatccagcatg
atgttaactgtgctctcatttttaaatgtgcaaatggatgtatgtgaaatggtggtttgt
ctgatacaatattggctgctaagacatgtctgacctagagcctagatgtgttggcttctt
aataatgctggatgatgtatatctggatcattctttttaattgactgagatgatgcgtat
cttctacctcttgagagttaggaatgtaaagggcaagtgaaaaatttgttagatgctctt
aaaagatgaaaatagatgtgctccatcactagtatagtttctttttgaccactatgtcaa
cctgtttagaaagtatattttaggccgggcgcggtggctcacgcctgtaatcccagcact
ttggggaggctgaggcgggcggatcacgaggtcgggagttcaagaccagcctggccaaca
tggtgaaaccccatctctactaaaaatacaaaaattagctgcgcgtggtggtgcgtgcct
gtaatcccagctacttgggaggctgaggcaggagaatctcttgaacccgggaggcagagg
ttgcagtgagctgagactgcaccagtgcactggcatggcaacagagactgccaacagagc
gagactccatctcaaaaacaaaacaaaacaaaacaaaaaacaaagaaagtatattgtaaa
catcaaattaaaagaatttcctgaagtaactttttttttttttagtaggggtggggaaga
tatgagtagaagagaaatgaattggaaactgagtaccactaccctttttcttaccttcta
tccagaaatctttcttaaattccacagtatcagtgtaacatcttttatcttttgagctgt
ctttgagattctttactttgtctccagactgtttgttaggattctactagagaagttgtt
ttcagattttaatttctctgctccattttaatcttctaattttttaatgaaaaaaaatcc
agttttattgagttcttttttttttttttttttttttgagacagagtcttgctctgtcac
ccaggctggagtgcagtggcacgatctcagctcactgcaagctccgcctcctgggttcac
gccattctcctgcctcagcctcccaagtagctgggactacaggcgcctgccaccacgccc
agctaattttttttgtatttttagcagagatggggtttcaccgtgttagccaggatggtc
tcgctctcctgacctcgtgatccgcctgcctcggcctcccagagtgctgggattacaggc
gtgagccactgcacccggcctattgagttctaattaatgcataatttgttgagttttgac
gtatatgtgcactcatgaaaccatcaccacaatgtatatatccagtgaatatatacatca
cccttaaaagcctctttgtaatctcttcactccccacttcgcaggcaaccactgatttgc
tttctgttactgtaggtcagtttgtatattcttgaatgttatgtaggtagaatcatataa
tgtgtctcctcttttgtctgacttctttcaaccctagctcaatatatttgagatttgtct
atattgttggatgaatgatcaaaactttgtttttgtttttttaactggggaatagtattt
catttttagtatatagtacaatttattttttgcccatttccatttagctgttattgctgg
attgggttattaacagttattgactattatgaataaagtggtcacaaatgttggtgcatg
gttttttatgtggacttgtgctcttatttctgctatgagtggaatggctgattcatatag
taggtgtatgtttacctgtataagacactgccaaacttttccggtttgtatcagtttata
ttaccacttgcagtgtatgagcattccagttgcttcacatcctcagaattcttggtatgg
acagtattttaatttgatttgttttagtagctgtataataatgtcttattgtggcattca
ttaatcattaggaaaatgcgaattatgttgaacatctttacctatgcttgtttgccattc
acatctttttctgttgaagtattttctcgaatctttgcttattgatttattgatttattt
attttgagtaagagtcttgctctgtctcctaggctggagtgcagtggtgtgatcatagct
cactgcagccttgaactcctgagttcaagtgatccccctgcctcagcctcctgagtagct
gggactacagatgtgtactacaacacttggctgttttttttttttttttttttttttttt
aaggatttttttaaagagatagggcctgactgttgcccaggctggtctcaaacttctggc
ctcaagccatccttccacttcagcctccagaagtgcagggattacaggcttgagccacag
tgcctaaattttttcattgtattgtttgtcttattattgaattataagagttctttctat
gtgctaaatactagtcctttggataattacttgcagatttttttcagtttgtggtttgcc
tttttttctttacccatgtcttctcaagttcaagtcttttattttgatgaatacacattt
atagtttttcttttatatttcatgctttttgtgttgtattttggcaggccaaataatgac
ccctgaaagatgtccatatcctaatttccaaaagctgtatatatgttacctgatatggct
ccacaaactttgcatatgtgattaagttaaagattttgaaatgaggagattagccaggat
tgtccaggtgagcccagtgtaatcagagagtccttgtaagaaggaggcaaaaccaatcag
ggcagaagagatgaatgactgcagcagaggttggaatgatgtgctttgaagatgaaggga
aggggccatgagctaaggccatgagataataaatttgtgttgctttacaccaagtttata
gtaatttcttaccacagcccatatagattttgcctgtaagtggaatgccgctgtaacaaa
tacctaaaaaggtgaaagtggctttgaaattggggtattggcagaagctggtagaatttt
gaggaacataatggagaaaacctagattatcttgaatgtacagatggtagaaatacagat
gttaaaggctctgctggtgatgccccagaaggaagtgaggagcacagtagagaaaatgtg
tatcgtcttagagaatacctaaatcatcataaagagactgttggtggaaatgtgaacatt
aaaggtgctgctgcttgtgagtgctttgaaggaaatgaggaagatgttattggaaactga
agtgaaggagatccttgtttagattgtaacagaaagtttacctgaattccgtcctgtagt
tatgtgactaactctcctgtgttgcctgggtatagcagcaatcatttatacatacatttg
cagttctgttctgtacttgttataaagtagttttaattaaagtgacagatttatgttttt
ataaattaaatttatatttaaaaaatcagtatttagaacaaatatcttagtggactatat
tcttgagatattattattaaagtctttttacagcttcctttcaaataaatggatatgtaa
tttaaacctttaatttctggatttcctgatgacttttctttaaaaagaataatttttaaa
aacacattttaatttataactaaaatggttatttaaggaaacacatatattagttttatt
tagatatatgcttttttattattaatttggaaagtggaggtgcattgtttggtagaataa
ttttaaatggtagctcttgttaatggctaatatttattagtggatacaacttacaaatat
ctgtacattcagtctttatccttatcttccatatctttttcttctgcattctaggttttt
ttgtttgttttttggaaggaggatatataaagtaattattactacatttttagctgttgc
caagttgaaagtcagtgtttagttttttttaaaaagggctatttagtgtggaattcctaa
atattatagttccctcttctgtcctttcagagctattgcaccctgttaaattttttttgc
attcagttcaaccagtgtttgaaaattaccaatagacttaaagtacttctctgaagttag
aagagctcctatgtctgatgctatattgctagtatttctattcaggttggaatttggaat
agtcattatcaaaagttatttgtaggcaaaagaaaacaaaaagaaaaaagttatctgtag
gaaatattggagactggggtacaaggtaagtgacacagtgaagaagcacagacaatttca
gaatatgagacacctaacatggtttctgcaaggtattggcgtggaaaaaaaaaaaaaagg
gggggggggcttagagagttactctccagtaaaagagatataagaagcagtactatagtt
atatacaaagggtagttctttggattctgtttagatcaaattaactagaaaatacatttt
taaaataagatggggaatttgaatattgactggtattacttaatatcgtggaaatatact
tattttgaatgtgtatagtgactttgtgagtatgaaaggcagtggccaaattatttagtg
atacataatgatgtatatggatgagggaagtaatgacgtgatgtctggaatttactttaa
aataccttggaaaaaatgaataaaagatagataaagcaatattgctaatatttcagtacc
tgttaaatataggtgacagtatatatatagaggttcattatactcctttctccacgtttt
gaaaaatttcatttaaaaattcaaaaaaattctaaaaaatattttttcttttagaaacta
acacatgctgaaaaaattcaaattttgttcactgaccacgtttttgctgccaccactctt
tcaacatttaaaaaatgttcttaaatctttaaaaattcgatttacattttcattaagaaa
atgaggcatttgctggccatttcatccttcctcttgtattttttctccttacctgctgcc
accgtttactagaaaaatatttctcttgttttttctgtaccttctccttgaaatataaaa
gttcagtgaaggcaggagagattttagcccgtttttgtatggctgcatcttccttatcca
tacctgcatagcatgtggtaggtgctgagtaaatatttgaatgactgaatgggtgactga
atagactgaatgaaccagagttgtagaagacatggggtgacgtggggtttggctaaggac
agaaccaaggagaacccctttgaaaattcactctgtgggatttacaggggaaaatgtggg
ctactacataagaaattaagataagaaatcatctttcctgtttgactgtaaagccttaat
atttcaagcatttaacatattctcagcataaataattaagtttttttcttcttttttctg
aaacagtctcactctgttgcccaggctggagtgcaatggtgcgatcttggctcactgcaa
cctccgcctcccaggttcaagtgattctcctgcctcagcctcccgagtagctgggattac
aggcatgtgccaccacgcctggctaattttttgtatttttagttgagacgggggtttcac
catgttggccaggctggtctcaaactcctgacctcaggaacccacctgcctcagcctccc
agagtggtgggattacaggcgtgagccactgcgcctggccagtaattaagtttttatact
gtaagttctctaattgtctttaatgtattcttgaaagtgttttcctcctaatttatttat
tactgcaaggcaatccgttcttgaggttctatttaaagtttatgtatttcttctcaggaa
aaaaaattacttaaaactagtggtttgtacagtcaagatgagttttagaagctgttctta
taatttctttttatgttaaacatatataatgtcacatttcccttttcctaacaactgatc
ttctttctttttcaacgtgtatttataagctagatttttaaattttgctccagggactgc
tgtagttgttctactacttaaaaaaagaaagatgttagtattgaacactattctgagaca
cagctgataacaattgtgctcaacaatgaagatggctaaaaattgggttttaaaaataac
agtgaaaattcagaagcattttatacttgctattctaaagtgagtatttttctaatctct
gctttaaaattactgaagtccttttaatgaccaacgctgtattttaaggaaaaaatgtga
gcaaagatttttagtgattctaaatttgttttgctctctggatctctttaaacttttaaa
aattattgaagaccccaaaaagctttgacttatgtgggttatctctcttgatatttaccc
tgttagaaaataaagctgagaagtttaaatagttttcatttattaaatttatttaaaaat
aacagctaattacatgtttacacaattaacaaagtctggcttacaggaagacagttggat
gctcatatctgcttttgcattcagtttgttaggatattacatgctctatactctgacaaa
aactttactttgtgtatactcaagagacagagtaaaaaacataagtaacatcttagtatt
atagaaataattttgatcttagaaactccatgtaagtgtctcaggaactcccagggttca
tattttgaaaattgctggagtcatctctcagaggctatgtgtgacaaatggtgtttttaa
atgaaacaataatttttaaaatatagtgtcaattggctaagtttttatttgatacttttt
ttttcttttttttttttgagacagggtcttgctctgtcgtccaagctggagtgcggtcac
agctcactgcagcctccacctcctgggctcaagtgatcttcccactttagcttcccaagt
aggaagtaggtgggactacaggcaagtgccaccatgcctcagtaatttttgtatgttttg
tggagacagggtttaccatgttgcccaggctgctctcatactcctgggctcaagcagtct
acttgcacccaactcagcatcccagagttctgggagccggattcttatttgatacatttt
tacctttaggggaactataaaaattagcttatgcttatttatgcaaataaatttgagcat
tgaggaaggatgtctcttgttcagggttgtgtttgtagatttcacatcttttagactacc
atttaagagaagtgatcaccttgtatcttaaggaaatgaaaatcatttgatagaaatgga
ttgttaaatatttttaaaacaagttttgacagttacttgcaattaaaggggaaagcaact
ttaaaatgcattagttaattgaagaaatactcaagatccttaattgttgttagtgagcaa
gtattaatgttctttcctctttgaaaacgtatctgtacctagacaaagttaattataaac
tttttcataaacaatattatgcttctttttcttctaatttttaaaccttatctttgttgg
tttgccttccagctccatctatctgcaacctaagaatcaacaatactatacatcaaatta
gctgatgtggaaggaaaatgataaataatgaaaatacgtcatattgcttatactctaagt
tataattaatataagatccaagggatattggtgtgggggacgaggaggaaggaacattgc
aagtcaagctatttcacaagtattctatacctttgaatcttttatttcatagttttcttt
agaaacaatagtgatgcagactgaatagctagtaagataactttcattaatagcagtatg
gcagggatttttaattgatgcttgttggtaatattagacacatgatttatttataggctt
ttaattagaaaactaatacaaatatcaaattgttatggacaggttatacattttttggca
acacaaatcctaccaggttttaatgcctggtacttgtttttacattagtatgtggtatta
attttttcattactactcttagcttccctagcttacccatttgctaggagtacatagcag
cactaggacatttttcgctaacgtggctggcatgcatctgaaagtgtgacagagcagggg
tatagctagattttagaagttcttttgctcatttaagaaacttaaacttcagatttgata
gtacttcagattctacataatgtgctctttcgatgaatagctgtgtagcagcagcacagg
aaaagcagagttcttggcaatacccagttcaattgcatctgcagatgaggatttaaaatt
ctgtgcagcagacagtacctccagcaggcgagtaaaaggttactgcattatttttcttgt
ttgttaccatagcctttatctaccaaaggatttttaaaaaattgaatgtgaagtgcatgc
ctgtttttagatattctggaaattgatttaagtataacatgaatatcaaaagggcagtgc
aaggtgaaggcagaggcagtgctggatctttttagctttaaaccagggaagaaattgagc
atgttacatacacaaagaagaaacctttcagtgtcacagacaatgggaagttttaaaata
cgtattttaattcaaggtgtttattatttagttttagactatcttccccaaaaagggtgc
tcttacattagcaatcattggtctatgaatgttcatttaagttacagtaatttgaagtat
gattatttgtgaagactataaagtatttgtattaatgcgaccctcccttgtcctggaaat
tgtaatgtggattaattacattgtataaacaaatacctttgtgcattattggcaaaggta
gaatgcttaagtataaaaggtatagtatagctagcatacttaatattgttgcttatgttt
tcagggtggtttgtggtttttaaaataatcttttaaataaaatgcagacaagtaagtggg
agaaaattcagtatcaaaacaatgaggtttttgatggctttttacttgtggtcagtacct
acctatatagccatatgtccttttttctcattaaaactcttctaacatttatgatagcag
gagcctccttggattatccagcttgtttattacatcattatctgcaccagtatttttgct
atagatttttattgttcattacttctcaagtctacataaagtatccagagggaatttttg
cttgtatattttattcagttaccctcctaccctcttccatcagtcctcaaagatttgtat
tagctactgtctacattttgtttatttaaaaaatacttaggcctatatttttgtattgta
gattgaataataagattgaaaacaattctgtaaattaatctatagtatagaagtaaaatt
tgttgattattcttactgtgagtatttggcaggcttcttgaattgtagagtcataaaagg
tcagcagttctgattggtaagttaaagaatccatccagtaatttgctccctgttgtttct
caaagcaatgcagcagcagttcttttccagatattaaacggttcttgaacagtgtgtgtg
gagacggggaggggtggagctcattcctgttggttgttgaaatcgtgtgcatttacagag
ggaacaggttggataggctgagctgaaggagtaggcaaacagatctaaactgatggaaga
cagaaagtggttagtataattttggggtgtcttgagtttgctcctttgttaagtacttgg
gctttaatataaaacaatttaaaatattaaagcaccttaccttctttctaaagagtagct
gggagacttctgttttaatatccagtttttcttggcgatgagtttgtggctggcatcttc
tgtagcaaagctgttgttttattcttttttagtagtgtttctttcagccagagagctgca
gaatgaggcagtttgacaattttgagtatatatgtgacatcaccagttctgattttttaa
gtcaaattctcatctaatgagcaagtcaatgtgtggctgtggctgcagcattatttcttc
tttaatgaagaggttgagccgttcgacctgctgctccgatttgttctcttgtagccatac
atcagaccagttgcagagattatccatcgaatgtcccaggcaaccaaaggtaactacata
gatttatttcaaataaaaatatgcaatgaaaatggatgcattatgactaagaccaaatga
ttaaaaataaaagaccaattaaagatgttcttagcagttttcttgaccttgcagtagata
tccaatatcattttgtcatcatcagattgtgtagcaatggaaatgcactgcagtaattga
ttttgtaataggatcaggtgatttactagctgcactgacaaccacttgcttgcttgctct
gagctgtggagcactctaatggatgttgtgatttcagcctggagtttatgtgagactgcc
ggccacttaaagcagcagcacttattttaaagattagattagtttttctttcttgttttc
ttcgtttcaagttttgtgagtagcctcagtaactttatggttaagttgtatgccttcatg
aaattttagagattatatattatttcattcatcaattcatagtctttctgctccatattc
ctagctaaatatcaacatattctggtattgaccatgagagcatatctctaaaatatgaga
gttattggtaactatgctgtgttatctaaatgaagtggaatattcctcacattcgtagat
ttcattagcttcagactctagctgtaattagaatgatggtaaggttcttgatctcttgag
ttgtactgcagttgttttgcatccttttttggtcttttactttcttagtggtttcatggg
taaggcaccatttaggaaatatgagttgtattacttctaagggatactgatgaggatata
taaagctattttaaagtagtgtttaaggatatagcaaattaaaaatctaatatcaagtat
tataaatttcaaagtgattattttaaaataatttttgttttccttttctatgccttttaa
acaaataattggttcaaatatagaagtgtaggaatattgctaactgtaaaatagaactac
tgtcatagaaactcagatgctgtcaaagactttgattacttaaaagttttgctgatggtg
ttagattagaaaagaaactctcttccactcccttcctcaccaatacctctacctcatgta
aagtgttttatacagactccaccacataaaaatactgaattctttatcattccctgtttc
tgttcttgccatgatagagacaccatttctctcaaccacatctaaaaacatttacaaaaa
ttaaataagattaacaatttactgtagtagaaacggggcataaaattgtcatcacatgtg
gtattcaaatcaccatgttaagagaactttcctttttgatgtactcaaatagtcacttgt
agtgtttgaagccttagtgtttctagaaagttgaaaatattatctgtgctagtctgcaca
tttcctttaattcagatactttaaacattaattatggaaaattgaaaataatttaaacac
tagtatttgtaatcttttattattcaacaggtaaaaagtttatagactctcttgacttcc
aagaaaaaaacccttctgtgaacactgatgaacataagcatgttaatatcatttaggatt
cggtcaaggatgtgtctgaatttcatatatattgaaaatgtttaatgatgggccaccagc
aaattaatcatggatatgttttactggagtgccgtctatgacagcttcttttcatgatgg
gttcagcaaataggaaacgaggaagtaaacaccagagtgttgactactttttataaaagt
tagaaagataactatgattggtctgtgattagacagtattttatgtaaaataaatgggca
gcgtgaagttctagcctcagtggagctgccttttctaaagagccctggatcgagcattaa
aagagttggatttaatttggctctgccattaatctatttgataaccttgatcaaattatg
tatgaacattttaaagtcccttaaacatgctcttaaaatgtccattaaaaagatttcaat
ttacccttaccagtgggaagtattagaatacttgaccttgaagctatagaagttagagtt
aggaagaaggatgaagtttcttaaagaatcaagttgtaggtgatgttaaaacctctcctt
tcactttttatgtctttttttgggggggtgggtgggtaacatgtttttgctaagaatact
gttttatctctttgatatccaatatttcctaagtaggatagtaattctggaaattatcct
agtggttaatagaatagacctgggattaaaatctggtgctgccaattttttctagacttt
ctaacaaagataatgtcattagggagtttattcagtacctaggatattttttagtaaaca
cgttttaagaaggttggccattatgttattggtgctttcttcctgtatggcctattagat
aagcgctgtgcagtcatctttgttgcctaggcaaaatggtttgtaggttcattgattgaa
ctcttactttggaccaagtgctgtactaagcactttggtttttttttttttttttttttt
ttttttttttaaagacagagtcttgctgtgtcacccaggttggagtacagtggcgcgatc
ttggctcactgcaacctctgcctcctaggttcaagtgattatcctgcctcagcctcccaa
gtagctgggattacaggcacctgccaccacacccagctaatttttttgtatgtttaatag
agatggggttctgccatgttgcccaggctgatctctaactcctggcctcaagtgatctgc
cccgcttggcctcccaaggtgttgggattacaggtgtgagccactgtgcctggcctgtac
taagcacttctatgttaattatgtcatttatgagaaagactctaatgattatttttaaag
atgagagaactgaagctcagagaggttctacaactcctaaaaatcacacaactgggaaat
agcagaaccaatgtcaaaactttaggcatcagcctgctgctcaaaggaagtgctcattga
agcgcttcggattttgaaatttctgataagtataatgcaaatattttttaaaaatcccaa
atctgaaacatttctggttccaagcatttcagataagagatactcagtttgtatttgtac
tttcaattaagctgtgagtgtagagatgaagccaaatttcatctgaagatgtaatataaa
tgattacctaatttttttagtcttgaataagaaatgatttgttcacctcttactcagcaa
taattcgtaggaaaaattattcatataaagcatatttgtactatttggagtaatgttttc
caaagtgtggttcagggctgggcacggtggctcacattggtaatcctagcgctttgggag
gtagaggcagatgggtcgcttgagtcgagaagtttgagaccagcctgggacacgtggcaa
aacctcatctctacaaaaaatacaagaaaattagccaggtgtggtggcatgtgcctgtag
tcacagccatgtaggaggatcatctgagtctggcaggtcgagactgcagtgagctgagct
attattgtgccactgcagtccagcctggacagcagagtgagaccctgtctcaaacaaaga
aagtatggttcaggaatcacttgtatctatcactttggatggtggttaaaagtaccagtt
tacatcagggcacattgaaagaatcaccggaatgggatccagagatctttgtttttaaat
aagcctctcacatgattcttggttataccaaattgttacaaccaatgatattgagaatgt
tttcctgttttaccttgtttctatgccttgcagcttttcctttaagaatttgtttacaaa
ccatagatggtttgtaaaattatccttctgacttatggtgaaatgtaaattggttctttt
gtggtactcattttaggaacttgtttaggattaaaagtattcataaactttgactcagaa
aacctacttctaggaattatagagataattcctaaggagatactcagattcgcaggaaga
cttaggcacaaggatgttcgtaccattatcatctataatggtgaaaaactggaaacaact
caatttcaaacatcagataaatggtttgttttttatatccatttgtttggactaaccatc
cagccataaaatgttatatggggaagattttgtaagacataggaaagtgctcatgattta
atgtaaagtctcgaagaatataaaattatggtcatacatatatgtacatatagtgcatat
ggtcttaggttaaatcatatgaaattaccgtttttgtaggtcaagagtagtagaatatca
gcattttcatgtagtgtaacctaacatggcacccatttttaaagcacatgtttgtagaga
agcacatgtttgtagagaagcacatgtttgtagagaacgctttgtagagaaggagctgga
aggaaatacagcaaatttataaagtgattatctctggatgatgatattatggataatttg
aaatatttcatctttacatgtcttatgttttcaaattttctactataccttagactcaac
ctttgaaataggacaaagtaaattaataaaacagggaacagttgcaaactttctctgaaa
aacattaaaattttttagaagattgtaaatcctttaagctagtataaggacccctttata
ctaatattcttaactcttgtattaattattgagtgtgtttatgcaaatggatcatatatg
cctttacctacccacccagcaaattttgattggattttaatctgcagtagaaagtgtctt
tcaaaagaagagaaaaaaatcaattttcaaagtttttaaaaagaattgggttcttaaaag
accgccaactatatgcaggagtcagttgggtagccacgttaagcacaaagccctgtttta
ggctgtgtggttttgatttttaattgtaatttaactgatcgataaaactctgtagattaa
aattagtaatttgtgtatttattttaaaatattactgtatttttttgaaaggaaaatgtt
tctcccactttcatacactttaatcgtttaattttaaagaaatagcttaattaaagtaac
agctaccatattctggttatctgccatacaccaggttttgggtgctttaatatgtttttt
gttttgttttgtttttttgagaaggagtttcactcttgttgcccaggctggagtgcaatg
gtgcgatttcggctcaccacaacctccgcctcccgggttcaagtgatactccggcctctg
cctcccgagtagctgggattacaggcatgcaccaccacgcccggctaattttgtattttt
agtagagacagggcttctccgtgttggtcaagctggtcttgaactcccaacctcaggtta
tccgcctgcctcggcctcccaaagtggtgggattacaggcatgagccaccgcgcctggcc
ttgggtgctttaatatgttacttctttcaatcttcacctagaatgtattaattagatatg
agctttgttttagcgttgaggaggcactgaaactattataagctttcctgagatcatatt
aagtggtggatctaggtttttacctcagtaatgtctaacttaaagacccatgttcttatc
cctgatactgtccttgttggtctgtctgaaacttttaagtgttcataacttgggaaacaa
aactgttgtataagccttcctaattcagactgtcaaagtgtagagcaaagtatataatga
gtcattattgctgatttttattcaactatttgttacctgtcctttaatctggtcagtaca
tgtgtactggcatttaacgatcgcctgctaatagctaacctcttgtgtaaaccactggaa
atactaaaacagagaagccaaaaattacttccttggagtttacagtcctaatgagagaga
cagacaacaaaatagttacaaggtatttcagaacataggtatggaatagaacctgttgga
acagaaagtttttaaactaataatgtttgggagtagtggtgaagagttacttgggaagcc
atcttggaagaggtgaatttagaaggagccatagatttcgtttttgctaccagcctagga
atttacattttaatacaagaacccctgcctccagagtgatctgtaattctactgaagttt
gagaaccaccactgtagattgtagttgatgctacgagaaagcatataataccctgaggaa
gctgtgtggagtgggaaatgagaagttggaaacctcagagcaacaacattgctttgataa
gtaggggaagttatatgatagttacgattaatatattataagcaatatcttttagtgcct
agaagtaagattggaaatgtcttgaattttgtataagatcacaaaacttacaggaagaat
atcaaaattccgattgaggttacttttttctgggagggatggtgaaatgttattctttac
cctacaatgtgtgattaagagtgaataaaaatcgtgttaaccttttaaagataatgttta
agaaacaggtttaatttttaaaagaaaagtgagcataggagcaaagttttgtaacatatt
ctgcttctatataggaaattaattcctaaattttggaaatgactagttgaaagtaatagc
agtctatcctccaaagagagctttttaaaatttattttttagaatcagacctgggttaac
ttttagatataatttggcataaccagcaagtcattggttattcaaaaataaagattggaa
ataattgccatatagtcttggctctttgcgtattttgtgatactgattgttgcttcctcc
tttacttccattttattattagagcttcattaagagtgaattgaaaagaaaactgaattt
tattattttaaaaatctgaatagttggattggggcaactttcttcctatttatgagactt
cactcatttatgttggtcttatattgacattaaagaatcagtgtcatatactggtgaata
ttaacaaagagacaagattaagattaaagaaattgaacctttaagcaggaggcgtgtgac
taatggagcaaatttctgaaaggcttaggggtagagaaaatcaaagctactgatggtatt
aatagtcttagaaatgatggagattacatttttcctctcagagacaggcgacaaagaagg
gaggagtaaagaaacaaaacctttgagattgagatatcacaagttgataggtacacagct
ctctgaaagcttcagtcatctcaataaggagaggaacatacttagccatataagttcgag
ttgctgaatatacattctatgtaggtaggtctgtgtaattttgatacgtattttctgttt
atcacagtggaaattgttcttgatcaatgtttttatttcaataagaaagatatattgccg
cttccatcttaaaatttctacatagaagagacatgacgcttagtgaacgttattgaaaaa
aaaaaaccgtataaacttaagcaatgaagaaagagaaacagtgtttcagaatgttacagt
attgaacctgaattggtagagagaatagaaagtggtgtgaacagtcagggtccaaatgac
attctatataaagcatataggacatgatttggagcagggagtgggatggggaatgcttag
attacatctctttactagatgggtgtacttgttctgtgcaaggtaccattttctcccctt
ttttgttagctcatatatcccttcccagttttgaagagtgctccactggtatttataact
ttagtgatttgacatttcaggatatacctgtttcagaaaatcataagaagttttaaaaat
gccttgatgtaaaacaaaatttcaggtaaacttaaatattttaagtttttaataatttgt
gttaccaaaattaaattttggttagagtttgacattcctaagtgatagaatcatcttaaa
cacatgttgtcaacagaaatttgaatgctgaagtcaatttttgttattgttattgttctg
cttagtgaagaaatgtatcctctctaaccagggacagtatgtactgctttctccttaata
ctttgagattttcataaagaaaataatttcaaaggtgttgtgctgcaagctaaagagatt
atttctaaaggaagaatttgtcattctaatggttgtcatcttccttgtggcattattata
aaattaagaaactgaaaaaaaatttttttagaagacatagtatataccagaatttaaatt
tagtttggcataagaggccacatattctacctatgctattacagtctgtatttgttattg
cctaattttgactcattttgaatatctcactccagtcctttttattagtatttcttaaaa
caaaaatgggcaatatgttggattgactgaaaataatttttaagtgtttagtagcaataa
aatttaatcttttagtgcagattggtttaccccataacagcacatatggaagaagtaaag
ccaggtctgtatgaagcaggaggtgacattggactttttttttttttttttttttgtgac
ggagtcttgctctgtcacccaggctggagtgcagtggcacgatcttggctcactgcaagc
tccacctcctgggttcacaccattctcctgcctcagcctcctgagtagctgggactacag
gtgcctgccaccacacctggctaatttttttgtattttttagtagagacggggtttcact
gggttagccaggatgtctcaatctcctgacctcgtgatcggcccgcctcagcctcccgaa
gtgctgggattacaggcctgagccaccgtggacttctttaaataaccatagtacaggcag
ttacttttgttatataacagtatagttgtctctgaagaaaacctgaagctctgcaaaatc
gggccctgaaaatcggtttctgagtggcttgttcctgtgtaaacttgtaatctctgtagt
aacgaaaccagtacccattctaataaaaatattatatagctaaatatttaccccagttag
ttctaagttctaggatttatgctttctcctttgaaatgtaggtcctcgagaacattcatt
tctacttatgatgaaattgtgaaaactagtaaatctgatccagggtgtagtttttatcag
ttgcctctcaccccttaaaaaaaaaaattaacgtgggaaaaatattgtcatggattttct
atctgtactcacagcttgaccagatggttttaacattgtggaatgcctagcagtttaaag
ccattaactagcctctggttaccctcttctggatttctagtttttttcttaaggtcatca
tgtattgcttaagctctgtctttgtgagaaagcttgtccctgctggctttaaaactttac
tatgctggggaaaattttggactaaagtgacattcacattatactgacctcatcgcccaa
atgatgaatgaatgaactaatgtacactacaagaacatgtcttaataggaaagtagactg
ttgtctgtagttgtcccattgttttatcattgctgctactatattgacatgaagtagcag
tgggtgtgcatgtttgtatattttttctgtgctgttaaataagtccacgcacatctccca
tgtgggtagggtagtactgggtgatggagtaggtttaccagtaccatttgtcccttactt
tacttgtatagctttatgttttaaaatagagtgtttttgtttttttgagacagagtctca
gtctgtcacccaggctcgagtgcagtgatgtaatctcggctcactgcaacttttgcctct
ggggttcaagtgattctcgttcctcagcctcctgagtagctgagattacaggtgtgtgcc
actcctggctaatttttatgtttttagtagagacagtgtttcaccatgttggccaagttg
gtctcgaactcctgacctcaagtgatccacctggcttggcctcccaaagtgctgagatta
caggcatgagccaccgtgcctggccactttttgttagaattaatactttgaagataccat
ggatttggttctagaccactacaattaagcaaatatcataataaagttagtcacaagaat
ttattgtttccccagtgcatataaagttatatttacatggcattgtagcctattaagtat
gcaatagcattctgtctaaaaagtgtgcataccttaactaaaaaatactttactatttat
ttatttattgagacagtcttgctctgttgcccaggctggagtgcagtggtatggtattgg
ctcgctgtaacctccgcctctcaggttcaagcgattctcttgcctctgccttccaagtag
ctgggattacaggcacccgccaccacacccggctaattttatatttttagtagagaccgg
gtttcaccatgttggccaggctggtcttgaactcctgacctcaagtgatctgcccacctc
tgcctcccaaagtgctgagattacaagcgtgagccatcgcgcctggctaaaaaatacttg
attgctaaaaaatgctaacaatcacctgagccttcagggagtcacgatcattttactgct
gtagtcttgcctcagtgttgatggctgctgactgatagagtgatgttgctgagggttgga
atggctgtgtcagtttcttaaaataacaatgaagtttgctgcattgattgactcttcctg
tcatgaacgatttctctgtagcgtgtgatgctgtttgatagcattttatgcagagtagaa
cttctttcaaaactggaggcaatcctttcaaatcctgccgctggtttatcaattaagtgt
atgcagtattctaaattgtttaactttgtctcaggaaactactttccttgctcattcaga
agaaacaacaccttatccattcaagttttattatgagattgcaacagtttagtcacattg
tcaaactccagttctaattctagttctcttgctatttctacgcgcctgcagtgacttccg
ccactgaattttttttttttttgagatggagtctggctctgtctcccaggctggagtgca
gtggcacgatctcagtcagctcactgcaacttgcgcctgccgggttcaagtgattcttct
gcctcagcctcctgagtagccctccactgaagtcttgaacccctcaaagtcatccacgag
ggttggaatcaacttcttccaaactcctgttgatgttgatattttgacctccttctgtga
aacacagatattcttaatggtatctggagtgcagttatccttctcaaaaggttttcaatt
tactttgcccagatccatcagaggattcactatgtatatatataaaacagctaatgtatt
tctaaaacaagactggaaagtagaaatgacccttgattaatgggctgcagaatgggtatt
gtattattaggcatgaaaacaacatgaatctccatgtacatctccatcagagctcttggg
tgaccaggttcgttgtcattgagcaatgagtaatatttgaaaggaatctttcattctgag
cagtaggtctcaacagtgggcttaaaatgtctagtaaagtcttgcaataaatggatgtgc
tgccatctaggctctgttattccatttgtagagcacagggagagtatatttagcctaatt
cttaagagctctacagtttttggaatggtaaatgagcactgttctacctaaagtcaacag
cagcatcagcccctaacaagagagtcagcctgtcctttagtttggaaggcaggtattgac
ttcacctgtccagctagaagagtcatggatggcatcttccagcagggggctgtttcatct
acagtgaaaatctgttgtttagtgtagccaccttcgtcagtgatcttagttagctagacc
ttttaggtaacttgctgcagcttctgcatcagcatttgctgctttaccttgtacttatat
gttatcaagacagcttctttttttaagcccgtgaaccaacctctgctagcttccagcttt
tcttctgaagctttctcacctctctcagccttcatagaattgaagtgagttagggccttg
ctctggataaagctttggtctaaggaaatgttatggttgatttgaccttttattcagatc
actcaaaactttttccatatcaacaataaggctatttcactttcttatcatttatgtgtt
cactggagtagcacttttaatttccttcaagaacttttcttttgcatttctagcttttga
tttaaagtgagactcttcctttttgcctgaatgcttagaggccattttaggattattaat
tggcctaattttaatattgttttgtcttaggggatagggaggcctaaggagagggacaaa
gatgggggaatggctggttggtgtagtagtcagaatacacaacatttttgccgtcttcca
tgggtgcggtttgtggcaccccaaaacaattacagtagtaacatgaaagatcactgatca
ttgatcatcataatagatacaataataatgaaaaagtttgaaatactgcaagaattacca
aaatgtgatacagagacatgaagttaccacatgcttttgaaaaaattgtgctgatagact
tgctcaacataggattgccacaaacttccaatttttaaaaaaatatactatttgtgaagt
gcagtaaactgcagtccagtaaaatgaggtatgcctgtaaaacaaacccgcagaaaaaaa
atcgtctactcagaagagcactgacaagtatcattgaattattatatctacaacttgcta
atcaagctaatgtagtgtgagttgtagatacaaccattttaatgcaggggttcctgagat
ctgaaaattattttaagtgtgcctccagtgtagaaagattgagaaaggttgaaagtgaag
atcaataatgggatgaactggaaatatgcctacctgataatacaaagcaatgagagaaac
atagtatcacttccgtcatattcctgcctgcgatgcataatgtgaatccaaaattagaca
aaccaaaattaagggccgttatacaaaatgaatggcttataatctttcaaagttgttaaa
ggaatgaaaataaagaaaggatcaaggaagtatttcagatacaaaactcaaatgggttct
gaggaatagacagtctgatatgtcagtgccattttccttactttaacatttatattgtga
ttatgaagaagtatgtcctttttataagaaatgcatacttacgtgtttggtaggggatgg
tgcatcatgttggaaacccctcccctcccctcccctcccctcccctcccctccccaggcc
tctcgttagtagtttccaaaccttttgcatgctgaacccattgctcaaatgaaggcagaa
tattaaacatgtaaaggaagagcttgtgtgtgggggggggggagcggggggaaaggaggt
tgggggaacagagaattaggaacccctgagtgactacagaacttctgaaaaccactgttc
tgtaggataaaatctaaactctgaagcacattatacaaacctctatctatacacatcagt
ttgtctgaaattgtatatttctcaacaatattgatctatttgtatttcaatatgttgtat
gctctcatatgtctgtatctttgcaaatgatattttcttctctttaacgatttttgtctt
accctgctggtaaggaagtcaccttccctctcccttctgtatatccttgtcaaggaacta
tggaaattgaatggcataggtcttgatacaagaacctctcttttctctttataaaaaagt
cagtaaatagtggtggaacattcaggagaaagttggggacatagagactaagaaggcaat
gcagtattattaggacagggggtaaactaaggcatagaggacagatttgatgaacatatt
atcttttttatatacattataagaagtagaaggctagaaaagtgaacatgcttgctattg
cacgtctcactttccaatttcatgaagaaagcaaaaactacttctactttattacttctt
tttgatgttaattggtaccacatagcaagtttgcattatagtactgagctttaaaatgca
aggatatactcatgtatatacacagacaataaataataggttcttttccacattcaggaa
gtgaaactcagctgtccgtacgacacttactgtccatatataggcatccctcagtatctg
caggggattggttccaggaccctcacaccaaaatctaggtatactagtcctgcagaacca
acatatatgcaaagttggtcctctgtatacttgggtttctatatcccattaatgtggaga
atacttttgatatgtggatatggagagctgactgtatttattgaaaaaaaatccacgtat
aagtgcacctgtgctgttcaaactcatgttgtttgagggacaactgtattctgttacttc
ctatagtttctaaaagcattaaccaacaaatacatgtaagatgcgtggaataatttttcc
atttcctagaaaatagtcaatcatttttttttcagattgtgaactagaaagcatagtgaa
tatagatttttaaacatgaaaatgtaagtgtccattttaaattgttatggatatttctgt
ttgctgaagttcaagactgaagactactggaaaaaatcctgtgactgagagcacatagat
tccttctttttaggtagagcgcaacagtcaccatgacttcagtgtttgcatatggaagct
aaaatctgtctgagaaagggcaaacaatcacaggagtcattgatttatttatttgttcat
ttttaatgcaaattttaattgagtgaaaaagtggtggaaaaaacagttgactaaagaaga
tatgagaagggttaatacatgaagaagtcctcaataatattagccaccatggagatgcag
attaaaaccacaatgccatactactttccatccacaagaatagctaaaattaaaagaagt
cagtcaccactgaatgttggtgaaaataggaagcagctgaaactctcttacatggctggt
ggaagtgtaaaatggtaccattcctgtggaaaactaattgacagtttcttagaaaaaaca
ctcaccctatgagcaagcactcctagttatttacccaggagaaatgtccacaaataggct
ggcacaagaatgcttatagcagctttattcagaatgaccaaaaactgaaaacaactcccc
aacattcatcaagagaagtgataaataaattgaggtatattcatacaatagaaagtacac
agggacagaaaagaatggatttctgatacatgcgacaacacgaatatcaaaaacagtatg
ctgaattaaggaaaaagacaacaaaagtaatgcagcatgagtcatataaacatcatataa
aaatgatgtttcaaaatccacaaaactaatctgcactgatggaacagtagttatctaaat
gtgctaaggagtgattagaatgggaatgggacatgagaaaagagggaactttatggagtg
atgaaaatattcaatacctttttctttttgagacagtgtctcgctctgtttcccaggctg
gagtgcagtgtcgcgatctcgtctcactgcaacctgtgcctcccaggttcaagtgattct
cctgcctcagcctctttagtagctgggactgcaggcttgtgccgccatacccggctaatt
ttttttttgtgtttttagtagagatgggggttcactatgttggtcaggctggtcttgaac
tcctgacctcaaatgatccacctgccttggccccccaaagtgctgggattgcaggcatga
gccaccgcacctggctgaaatattctgtgtcttgattaaggtgttgtatatatagatata
tatgtttttcaaaactcactgcgctttctactttatataaaaagtacctcaattaaatta
catattaattgaaggcaaattaaaaacatctctgtctgtaactctcctactgtgagtctg
ttccatgttgaagaatgctcttgtcttcttttctttgctcttcttggttgggacttccct
gttctaatatcaggtatgctcacgttagaaagctaggacagaggcctagaatactgattt
ttttccatttcaacaggcaaggtgacagttttcctagagttcacaactcttggtattcca
agtcagcacatagttcacggtgaaacattctctaggaggtagagagaattgtgaccgaag
aatatgataggaccaaataagtagctatactagaaggaagcacatttgagaccactaccc
taaacagccaatgactacagaaccagagaaaaccagaatctgccaaatttcagaaataac
tcagcatctttacagggagcctctacacaatcacaatgagactctgaagagtagcaagta
ctgattcaaatccttttagctgtggactccactctgcctcacaatcattttagaagaggg
ttttgagctttagaataagcccaccactgtataaaactagtgagtctgttttttaaggta
ggattcccatcttagtaatcataattttggcatggcaattattaaatatgaaccacaagg
agaaaaaaatgtcctcttgttattactttttaaattaggcattttaatttcatttactac
tgcttgatactctcttctctgggttcctgggaggtttcatttggtaagtattgttcagta
aaaattatgggacaccattgttttggactgtgctcctgccctaggccccagtggaccaga
tcaaactaaaatgtagtcattcatgttaactgccacataatcaaatagaaacttccagga
agcagacagatcccaaagcagaccatttttttccctgagaacaggagattccagtctagc
tgaatcccctgtgctttaaacccttagaaaaagtaaactgaagtaacctgatattaacaa
atcagcttttttttcctgttctgtttccttgttcccaccttacaaaacccactgttgtgc
tgttgctcagtaggacctctcattctaccttgcagaatgggggctgccctgattcatgaa
atgcaaataaatgccagttagatctgtaactaaactgttgtaattttgtcttttgacaga
atgcagttaaaactcaggtatatccacactgcttttatttcgtctccttcccatttcaga
aactcctgtctagatttcttttcttgctctctaaggcaaacaattaccacagttagatga
atatgcctggcaatcgctattgacagatagactctcagaaacactcgttccattttcctt
ttcaaattcttggcaggaagactgggtggatatattgaaggaggtactgtttgattgtac
tgcacacagtgtggtttcaaaaatgtgtatttgaattggaaaagagaaatatatgtcagg
cactggggatacaatactgaccaaaggtatataccctgaatccaaaaataaatataatgt
agttgctgtaaacatattaattttaaagattgtcagaagaatatccatcttagttagacc
tttgcaagatatatagataagaaaatttctgaaagttacataactaaaatagaaaccagt
caaaatgaacttcattctaccctggtttcttccctctaatgttcttatctgtcaatactg
tgactgaaaaagccaaacctaccaccaccaccaccactgacaaaatccaaaaaactacag
atatctacaacattaattctaacaccaagtaacattataacccacttaaaactgttcttt
tttctttctttttttagtacagacagaacttttttttttttaatggtaagaaatatcatg
attttcttataaagaccttctgtatgtacaaataaagctatactatcatagtggataaat
cagtaaacaatatcttatgtaatctgtaatttttcacacaagaaatttaaaaagtttttc
aggttatagaacagtatttctggacacacctaacaaggcagtagctgacattctcaaact
tcaaaacaccctcactttcaatacaaataatgctgtgttgtacatttaacctaaaactaa
gagaaagcattgttggttggccacaacttgaatttcttattacaatgaggactgcttcag
gattttaccactaaactcagccgttcagagaactaggaagatagtaaccatggtcactgt
tcttcaaaagcactgatttcagatttgttgaagggatatttcttagatcttcaagtttct
gaaaaggtttccttcattctggttttctggatttccttgtcattctgaagttattcctct
tgcacaggcaaaattagagtactcattcaataacaacaacaacaacaacaacaaagccat
aaaatatgccttcttttttctacaacaccaatttagctactcaagatacagaactaaaat
attagcagaaaatcttgtaggtatgaagccagttatgtattcacattagtaaagatgatt
ccactgcattcatttaatcagcaaacatttacagaatgcctactacataccaacaatata
ttagtgactgcatattcatttaatcccgaactctgaaatcattattcctggtttacaaaa
taggaaacaggcttggattaaggttgcttgcccaaagatttatagttagtaagattcact
accaagtttgaaactcagatatgtctgagccaaaatttctgctccttccactaagccaag
ctgtcttccagcatggcttccagcatacttagtatcttgctgtatcaagagaaaacaagg
tacaggtgacagtgacgtgatgccattatgactatctctttttatataaaaagacccttg
ttgagataactacaactctctctttctcagacctcctacacattgcttttccttcaatca
tcagggtcatgagccttctcccctctcctcccccgccgccccccaccacccaacttcccc
ctaacaaagtaaaagaaatcaaatgagatttgattaaagtgttttgtctagactgcttca
ggactaaagggtcttggtaatttgtaccagcacctcacttcttccccacgcccaaaaact
cacgtacacagtttgattgttcttgctttctaaagtgttctcttttttttgagatggagt
ttcgctcttgttgcccatgctggagtgcaatgacccgatctcggctcaacacaacctccg
cctcccaggttcaagtgattctcctgcctcggcctcccaagtagctgggattataggcat
gtgtcaccatgcccagctaatttttgtatttttagtagagacggagtttctccatgttgg
ttaggctggtctcaaactccctacctcaggtgatccacccgccttggcctcccaaagtgc
tgggattacaggcatgagcccggccaaagtgttctgaaagctatgtgtgaatctgttaag
tacaatctatacttacatttgattataaatattttgacgatctgaattggtaagatttta
ggcaaaacaacaaaatcaaatcccgtggactctgacccaaagaatatattcagctttgtt
tgaatatatttcatcttgcttccttaccaattctcaacacaactccagaataactgttat
taactcttattgaatcttattttgtactaagccttgttaaatgttttacctatgaattgt
gatttcattgttacagcaaacctatgatatagatgctatataacagatgaagaaatgagg
ttcagaacctgtgctacgctgcctcgcaggcacaatccatctgataaaagtagtcccttg
aatctctctttctgaattccagtcccaggacagtggaaatacacagtattgtggtcaaag
ataaaccattcttggctccaggtgagctaatggttttagataagaataaagaatatttat
tagaaataaaaaattttgtaagtataaaattagactaggaaaaagctcccggttgaattt
tatatttataaaatcctttactgaagttgaaatcttctccttgaaatttctgcttggtat
ggcctctctgtttgcctcacaagtgaaatttaatcctaatgttaccttattttccaagca
taaccacaccagtaggctgctttgtcatgagtttagtatggtgtcagtagaaacgagacc
agcagtcattagggttacctaattcaaatcttgactcattttgggtacttcctacaagat
tagaataattatcttccaaaccaaaaacatacccaaggttccatgggaaagggtgtagct
tcaactctttttaggtggcaagaaaatcattctctaacctctcaaaggtcaggaataggg
tcgatggtccttaaaatttggtctggggaccactggttgctgaaccttttgcaggaggtc
tgaaaggtcagacctgttttcacaatacatgttaagaagttaatggcttttttgttgtca
ttcttttccaagtgtatttgattatacaacttgggatgatgttcctctgatagttaatgc
aattatacttatgttttaaaagtttgttagttttgatttctaatacagtaagtctcaaca
gatagaactcacataacaaaacacttttgggagtccttcataatttttaagagtgtaaag
gaggttctaagaccaaaaggtgtggaaactgctcttctaggtttcagaattatgaagcat
acaaagactcttcatctggaagggtttagaggtaattttatctaatccccttattttaca
ggtttagaaactgattctagacatttaagttcccagactaatgtcacagaagctaatgaa
ttgcagaggttaattggaagcctggtcttaacactcccaggttatcttaatgagttcatg
aggatggcatatggataatgcacttcaaagggtgttgtaagtattaactaagttaataca
ggtcaaatgcatatattagcactcaatgcacggccattgatcaataaatgctagtggttc
tgatcagtgagaatctaacctctgcttaaatacctttagtcatcagcagcttccactccc
tgagtaacatgttgcatttcttgatcaattatatttttacagaattcttcctttactgaa
gttgaaatcgtctccttgaaatttctacttggtatggcctctctgtttgctacacaaata
aatttaatcctaattttatctagcttattttccaagcataaccacaccaatttcattaaa
tgattcctcatgtggcatgactttaaactccgtcaccatcctatttgtttttctcaaaga
gctccagttgactgctcctgtgaaattgtccatctattaatgtaaatgttttttctaatt
ttacagagctccccgttgtattgtgtacagtgttaaaatagttttctgagatttcttgac
tctgttttcccaagtttcttgtggcccttctctttccttcgtctctattctgtgcggttt
ttatttcactcccacagtttctcattgctgtgaggccctgttatggaatgagagccctgg
ttttgaaagttcacagaggctagacttctcttgtccctgtagtcctggctgagggcccac
tacacttgtctgttatccgagtgggcaaacgacctacccgttttcatctgctgggcggcc
ggttatttggggggatccccctgttacaggtctgatctctgttgcttcctttgggaggcc
gaggcgggcggatcaccaggtcaggagtttgagaccagcctgaccaatatggtgacactc
ctgtctctactaaaaatgcaaaaactagctgggcatggtgacgtgcgcctgtagtcccag
ctactctggaggctgaggcagaagaatcgcttgaacccgggaggcggaggctacagtgag
tcgagattgtgccactgcactccagcctgggcaacagagcgagactgtttaaaaaaaaaa
aaaaaaagtttgtagccattatctctggaaatatttactcttcctcattcttctgcttat
ctctttctggaactccaactagatatatactagacctttgattctatttttccacacctc
ttaacctgtctagcttatttagtcatacatctctcaagctgcattcttccttggagcttt
atctgtaggaattctttgagccttgattgagtttatattgcttcagagaaaactaaggtt
tgcttcagctagttgcccaggaacattaccacctggttaccacttttaattaaggtcact
gcttgaggatttcagagcgacacagagccctgtatgagggcctgtttatggttataaatt
cttggggtgtaggcaggggtggggatcctgcttctttacctggagctaaaactgagacag
aaaaatccctgactgtccatttttgtgtggtgggtttattttctgttcaccctgaacaat
gataggtgttcaccttgtagagctccccgctttatgtgggggtttcctattagagaacta
tcatatgtggcctgttggcttattttcttgccttcagcacctgtgccagtacataagtta
gaaacccaaggtttccaggtttcgaaaaactctcagggcaaaagttcgttttagcctatg
cttagctcccagggtcctttgtacttggccatatggatttctttttttttttttcttcat
gtcaacactgtggttatatttatctgtctaacctactcttttaatttttgttttatctgg
tattttaattgtttcatttgagaaagtttttcatagtatctgtttggtagttggccattc
tactaggagtttaggagttatacttaactccttccagggagatgttggaagatttttact
aaaaatacaatatttaaggtgggtattgaagtactatcagaggggacctggggtggagta
tgaaaacaaaacaaaaccttacaaacgggaataattaagagttaggatatgtgagatgtt
tggttttgctgggcttaaaatagaattgtggggtatggaattgctgcaaatgaggaagat
ggactcagagatgaggctcaagagaggccacatgcaacttttgagagtctttgtgtgtca
cagtaaggaagatgaacttgtgaaatcaccaaagggtggaatatataaggaggaagctga
cactgtcatatctatgccttaggatgtttattttggcagcagtgaaaacaaatcaggtag
acaagagtggagcattgctcttcaaaatttagtcattgactggcagtaatggcatcatct
gggagcttgttagaaatgcagaatctcgggtcctatttcagatttaccaactcagaatct
gcattttaacaagactcctaagtgtttcatatgcatagtcagttgatgtctcaaccagtg
acctgcagagccagtgatctgcaaagcaagtgaacgttagctttattttaaatttcaaga
aagaagtcttaatttgcctagagtacatatcatttgatgttcacagtgtcactttatttg
cctaataactagctgactctagatccttagcagcattagatttagtatttgaggttttga
ccctatgtacctttgataagtagtgatttgtaacttatgaattaaatttgaattccttaa
catgttgctagttacaaaactctagtgtctctagcagactattaagaaattgagcaggtt
tcgttctccattcactgttaacacatgcggtaactcaccaggcgcttaaaacaagtgggc
tgtggtgcagggtacttcttgtttagtgacagaaatgaaaagatctaagaatatgatggt
tttaaataaatatgataagagatgaagagagttttaagtaatagaactattctaagtttt
ggatgtttggtttagtgtctctagctatatttctgtttaatgtcaaggcataatatattc
gaaccattttatttattaaatttaataaacattgattgtcctattagtcaaagtttcaga
aaaatgcaaaaagtcttgttctcaaaggaatgctccctggaccatgcacatcaaagtcgt
gggtggctgcttgtgggggtagaaaatattaaaaatgcagaaactgggccctactgccga
atcccaatgtacattttaaaataagcagcttcctggccaggtacggtggctttcacctgt
aatcccagtattttgggaggctgaggcaggaggatcacttgaactcaggagttcaagcct
gggcaacatagtgggacccccatctctgcaaaaaatggaaaaaaagtaaaagcatctccc
aaggaattctgccagcaggaaagttgtgttttcccccctcaaatctggaaaccataattg
ttcactttttgaccttttactggaatacagcatactctgagaaaagtaacaacactcaaa
taaaaaaatagaacattctattccagaacctcctctagtgactccttctagaaagcagtc
tagcaccccacccctccaaacacaaacataaccaccatagtgacttttaacaccatagat
tagtttttccttttttgaactctttaaagtggaataatacaatttatactctgtctgttt
tgtgttgtttttgcttagcattatgtctgtaaaatttgtctatattgttagctgcagttt
gtcattctcattctcattgctgtgtggttctctgttgtttaaaagtaccagtggtaaaat
tatcatttatgtggtttatctctgatggacattagaggtatggtggtaccactgataggc
accaaaatcaagtttaataaccactgttgtatattatttgggaaaatgtgtactcttgac
atgtttggcaaatttttatttctccaatctaaaatttagaaaattataattttaaaatta
tcaataaaaatttacaaagtttttactgtcaaatattatctaatagctcaactagctcat
gctagagtaatacagccatgtaatacagcattaccaacttttctaagtggtctatagcac
tgaggaattaaatagcaaataagggaagcatttcttcatgaaatcatttcttttaataaa
ttaaaaagaaatgagagtattagaataccaccattttgccaccttcagtgtattaatgga
tggaggcaatgaatatcaacagctgctaaaatcataagaaggaaaccagatgctagatga
cacataatgaagtaacacattattagacaaaggaacaaaccaagttcaataaagcctctg
gatccagcttccaatttgccacaaataaagagaccagatgaatatgctgaattgcattat
gtgtaaataatcagtcaaatctacactacgaaaagtcagatcaaacataaattttaagaa
aaagaaaagaatggaaaataaacctgtagattaaaagagactgaatagaagtagcaactc
ttcttaaaaatggtcaagattaaataatagttatctagggaggcttacttagctgataaa
gtgataaatacaggatgtaattaagaataatttaaaagaagtgagttacttagaggaagg
gaaggggctatgactgaatcagcacttggaggagcttctgggctcgctggcaaattgctg
tgtggtggtcacaagaatgtattctcttaatatgctaaggtataaagtttttgtatagtt
ttctgtatctgtattttattttactctaaaggaagtagttttttaaaaaagattatagat
ttgctaatctaatataaactcccctagagcttcatcagtaatagtctagtgtcatctgga
ttatcttcagcaatcattttcattctaaattgtcatagtgtcatctcaaagcagaaagag
acagagagtttgacaaagtgtcttaaaatgcctgatgtctgtcttaatgattctaataga
aactcttcagtacaatactcagacttactgcgcttacaactacataatggcttataaatt
tgggaaactcaacagatttgggaatacaacacatatcttgaagccagttaatttactttt
tacatacctcttcactattccactaccagtgtgcaactggtaattcagttgaattcataa
agaattcactgagtttttctgaatttcttctgatttcaagaggctagaagaactcagatt
ataaagcatcaggttatcacttttactatataggcacaccatggtttattgcactttgct
tctgattttttttttaaacaaattgaagggttggagcaatcctgtgttgagcaagtctct
tggtgccattttccagcagctcactgtgtgtctctgtgtcccattttggtaattctggca
atatttcaatctttttcattattattatacctattatggttatctatgatcagcgatctc
ttttatgttactatcataattgtttggggatgccatgaaccacacccatagaagaataaa
agatggcaaacttaataaatgttgtatgtattctgaccactccaccaaccagctgttccc
atctctcttttctcaggcctttttattccctgagacacaacattgaaattttgccaatta
ataaccctacagtggcttctaagtgttcaagtgaaaagaagagttgcatgtctctcattt
taaatcaaaagctagaaatgattagtgaagaaggcatgttgaaagcagagacagggcaaa
agctaggccttttgtgccaaacatccaagttgtgagtacagaggaaaagttcctttcttt
ttttcactttttcgtatgtttaaaaagtagagacagggtctcgctatgttgatcaggctg
ttcttgaactcctgacctcaagcagtcctcccacctcggcttcctcaagtgctgggatta
taagcatgagccactacacccagctgaaaagttcttgaaggaaattaaaagtgctactct
ggtgaacacatgaattataagcaaaacagccttattgctgatatggagaaagttttagtg
tctggatagaagatcaaatcaaccataacattcccttaaacaaaagcctaatccagagca
aggcccgaacttacttcaattctatgaagggtaagaggggaggaagcttcagaagaaaag
ttggaagctagcagtttggttcctgaggtttaaggaaataagctgtctcgataacatgta
agtacaaggtaaagtagcaagtgctgatggagaagctgctgcagcatgttatctagaatg
tctagctaagatcactgatgaaggtggctaccctaaaagattttcaatgtagatgaaaca
gcactctattggaagatgccatctaagacttttctagctggataggggaagtcagtacct
ggcttcaaaacatcaaaggacaggctgactctcttgttaggggctaatgctgctggtgac
ctcaagtggaagccagtgctcattggtcattccgaaaatcacagggtcctaagaattatg
ctaaatctactctgcccatgctctgtaaatggaacagagcctggatgacagcacagtttg
ctagatatgttaagcccactcttgagtcctactgctctcagtgaaagattcctttcaaaa
tattactgctcactgacaatgcacctggtaacccaagaactctgatggagatgtacaagg
agattcctattttttcatgcttgataaacataataacatctattgtgtagcccgtggatc
aaggagtcatttctactttcaagttttattatttaaaaaatatatttcttaaggctgtag
ctgccgcagtgattcctgtgatgaatctgggcaaagtaaaccaaaaaccttctgggaagg
attcaccattctaagtaccattgagaacattcgtgatttatgggagggggtcaaaatatc
agcattaataagagttgggaagaaattgattctacccccatgggcaactttgaagggctc
aagactacagtggaggaagtcactgcagttgtggtagaaatagcaagagaagtagaagta
gaacttgacacagcgtgacaattgctgaaatctcaagataaaatttgaatggctgaggtg
ttgctttttattgatgagcaaagaaagctgtttcttgagatggaacctactcctagtgaa
gaggctttgaaaattgttgaaatatcaacaaaggacttagaatattacataaaattagtt
gacaaattgataaagcagcggtggtttgggaggattgcctccaatcttgaaagaagatct
acagttaggtaaaatgctgtcacatagcctagtgtctttcatgaaaggaagagtcaattg
atgtggctaactttattgttgtcttattttaagaaattgccacagccatactgattagtc
agcagccatcaatatcaagataagaccccccaccggcaaaaaaattggactcactgaagt
ctcagttgatcattagcatttttttaagcagtatagtccttttaaattaaggtatgtaca
ttttttttacacataatgctattacacacttaatagtagtgtaaatataacttttattgc
actgggaaaccaaaacattttttgtgacctgctgtattgcaatattacctttattgcagt
ggtctggaattgaacctgcagttttctgaggtgtaagtattggtgaaatcattaacctcc
tcctctgtgtgtgtggtttcttcaactcacaaaggatagcagccccagtgaaagtcgtta
cagaaggcagataaactccattttttttaagactttgccactctgtgtgtgtgtgtgtgt
gtgtgtgtgtgtgtgtgtgtgtgtgtttaccttatcctagtgggccttaccacacatttc
tattatgtgagatccaaaagtgatgtggtatacacaagagtaattttcaccccattctta
aaactgaaaatgatgtttaaatcactttttcttgggggaaaattcaaccccgtggtaatt
aagcttcacttttcctgaaattaatctttttataaattttataaacttataagaaatgta
atacattttgcttaagtaactagcataagccaaaatggcacttcatttttcttaggctca
gcagatttaatttaataaagattataaattgattttaattaatgatgtttacgaaatgaa
aattaattgacagtgttgaatagtaaagctgctaaaaccataagccaaagcaatatgaat
aaaagtataatttagacacaattttaaaccagattttgaaaaactttactcggtgatatg
gtcaggctcaattgtatgcctgctagttagattacttcttgtataggctgatatatgcat
aaaattattatttttgaattttaaataaaattatatttatctctttaatgtgaatttacc
tctttaatgtaaaaattatatttgttttttgtccttgtttcttactgcacatatcaattc
caggtcaagcatgagataagcaaatcatgttaatttcattttcaactgaaataagactat
ttctgagcttattcttgtttgctgaataaaaaaagattgttcatatgtgcgagaaattgc
tgcagcataatgttcactgtttttctgtgagtagcaggataatcttttcttcatagaaat
ctacatcttgtctgggaaagattagtaaaactcatctttaatgtatgataaaactgcaga
tctttttccttttttctcaagtcaaattttcacagtcagaacattcagaaacaggatccc
ttttccagttctacacttgtgttcaagtgaaggaaaatctgttcagttttttttctgcag
ctcctccaggtgatttccagtaagacttgagacttgctgatatccttcctcactctcaag
cccaaacagcaatagaaacatttcaagattttcttttgtaacatgtttttcaaaagctca
ttttccttttacatccaagaatgttgtcctttgaaataaagaaaacccaaccattttctt
cacaccttacagagactgaaaaataagtactacgtaggctagtttctacagccattaatg
tttaagtattcaacagactgtagcctactgttttctttaaaggacccatttcagttcaaa
taccctattgagaagttttccactgctaagtggattttgtgtgtgtgtgtgtgtgtgtgt
gtgtgtgtgtgtggtaagagctgttctttaaatgagtgaaatgacctttgtttaatgaag
ttaaccattttataacatctttcagatgttaaaacattttgccccaagagcttttgatat
cagtactcttaacacagatgttttttgtgttagtttgggtcttctgagacacagatgtca
agacaagatttgatgcgcagggaatctcccaccccaccctactaccctcctattgtcctt
tggagcaatccccattctagtacttctgccctgcttggtagcttgctgggagaagcccag
aggcagcaaggccaggggtggttgccatggattccaaggtgcgatagatggaggctgtca
agcagttctgttcctcatagcaggttaggttgaggggatatctgagtagggtaccactgt
ggccactgcaccctcctcttcagcctttgacatctttccctcatgtggacactctttact
gtgttacgcacccacagggatgttgtcactgtgttctgcctttgacctacgggggcttct
ttactatatggattattcctcatgcttcttagagtcaagctttgccaccttgcccccttg
ttggtctgcctctgttcctccccgccgccatccctcactacagaaaggctcttttcattc
tgctttgactcccaaattttggactacctgcctgccacatacatacctaacttgcttggc
tccatctaatagctttttgactaaatttttgaggaaaagaaggaagaagagccatctggg
tttcttacatctgcaactagctagaatatgattccattcactgaaatagagaaaataata
ggaggctctaagaacaaatttgggccttacgtatgtgtgcatgttgtggggaaatagaag
tttggatgttaaaataatgaatttggtgtgactttgggacaccttcttgaaattaatagg
ctcagtagaggagtctgactggagataatagtatggggagtcattagcttctgtgtgata
gttggaaccctgactttcaacgaaacaacaaaggagggaggagctagcgatggagcacag
taacagtgctgcctgagggatgcactgaggaatgtaccaaggaaaattaagggacaggag
agagttgtaaagagagctagtgaccaacagaatataaagatacaaaaatggcaaaagagt
ctttgagatttaacaaccagtttaaaatttagttattatggagtaagcccatatcacaca
ggtggtggcgtatcacacagggattaaatttagccaaaacaaaatatgtattataattca
aataacaaaaaatatatattgtcatgtggactgaggaatagtcttttgcatcctttccag
taaagatatatatgattgactagtgaagtctatttaaattattcagtgtacatccataaa
actttatcaattactttctgtgtatatgatgacacttgccagtattgcccattttgtgct
gattttgttagtgtacattactaaggagcttttcatcaaattttgcagtccctgaaagat
tttcatctttaaagttacaaatgtgatttctcagctgcagtatagaatatatggaaccag
aaatactcaaaaaataattgttttcgaagtattaatctttttagtcacagttgttttttt
acatcctgtgctgaaaataacaagaaatttgatacagagggaaaaatgatttattatgac
ttagtagacaatttacaaatgtgtgttaagatattaggatatatgtcctaccagtttcaa
aagtactacaagtgacttgttcaatacagagtacaactttaaaaaatgtatttaaagtaa
ttgacgttgtgtttgtgacctggctcgaagttcttttttggtgaagagcatgcttagtac
atgattgtctttaaaaagatctgataattatttactcagtaaatgttttgagtgccttat
gagtcaggcatttttgtgggtgctggagataatagtgatgaaaaacaaaagtcccttttc
cttatagaacttaaattctagtgtgggaaggcagatagtacagaaataaattgatggatg
gatagatggataggtaggtatttgctatatagcaggtactatttttgcttttatatagtg
ctgtggaaatgagtaaagtcaggacaaaggaaaagagaaggtgatgctggggggtattat
tttaagttggatggttagggaaagtatcattgaataggggacatttgaacagagtcctct
atgaaggaagtaaaagaaataaaaagaggaatagctagcacaaagactctgaggttgcag
cacactttacccctcaagaaagaacggtgacagaaagggtggaacaaagtgagagagtag
taaaatatgagttcacagtggtggggatgtatgtagatgatagaaggacccaacaggcaa
ccataaaagacattggcttttactctgagtgaaatgggaagctcttagtgggtgttgagc
agtggagtaataagataaaacttatttcatcatgcatttcaggtacttactcctaatcat
agtaattaataaattattaatttggggtatagaaagttcatatatgaagtggagggtgtt
ggctcttttaagaattcagtgaaaatcctaaacttcttgcagaaagtgattccaagcttt
gagttattcttcctgatgatcagtacagttggtttactttcttgttatctttgtcctaaa
gcttcttaaatcactttgtagctcaagcctaatggattatacctgcccatgtaaattctg
aaaatgttaatgttgcctagtgattcagaggctttagtaatttacttaaactacttcttg
ttttattggtataaactgtattcctcagtgtctactatgatttcaaagttagtatttgcc
ttggaatttttctttgaagctggcaactctagttcaatataagacaggctctcagagctc
cacttatttaacaactgtatctatgcccacttttattcctctaagcattcctagaataag
catttcaaatggttttgaattatcaaaggtctttttgaaaaacaaggttattgagaaaat
gtttaaaaagttcatgagaaagttgtctaaaatacacccccctcccccaaggtaggcctg
aaatgccactaaatcaacaaaggatagtcactaaaagttaccctgtattttctaggactt
ggggaaagtcatggagtagctgtgctttctctaagtaacctattttaaatttttttgaag
agcaaaattattatacctctttttcatccagaatggagctgactgtaggcatctttttaa
agtacccataggtcaggacctctctggactttccactaccttgaaagacactgccagaag
aatgttgttccccacacttaaaaaaggaaaagtaatgttcttttctccagccattatttt
atgcaaagtccaactttattttatctgagctatctgaactttcaatttatttgaaacatt
tattgattaatatctataatggagcttttaatttctctgatcatttatcctttgttaggt
tagcagaactgtaacaacacagtcactttcacagccacgaatgatacttttatagtcatt
tgtggacatatactgtgcagagtatcacaaagtttgagccacctgacaaacttatttcca
gttgagatctgcagggtgatgctctgccttcttgttgcagctctcataccatgaacaagt
gtcattttcatggtccatgaggaaaaagcattttcctcattttggtgctttttgttagtg
attgtgcagtttagaatggcccccaaacatagcctgaagttctgtttaatgttccgaagt
gcaggaaggctgtaatttgccttaaagataaactaatgtgtgttaaagaaacttaattca
ggcattagttataatgctgttgaccatgagttcgatgctaatgaatcaatgtatattaaa
gatatatttaggcagaaacacgcataaaaaaggttatgcattgatcagttggcaacagtg
ttgtgaccagaggcttgtgagaactgtatttcccctaggagcaatggctcagtatttgtt
aattcagtggttgcagcaattttatagaacacaattactgtgaatgggaatcaactgtat
ttgattaacggtggtgatttatagggactttagataccagaaatgtttcataattccctt
tctctgaaatcactgttttaatatgtttaaatgcatgcatgcagagttgtgatgttaaac
ataattttactgtgggttctggtcaaaaaagatgaaatgtcattggttaacttgaaagag
acattattctaaacattatcctccagaaaattgacatgtacaggaagcatattgaatgct
cagcaaataatttgttttagagtaataatctttgagtattgtagtgattcgagttttgga
aaagaaggcagtatcagtgttagagaataatattctttcaggattttatgtagttacttt
aaggtgcatgaaaggagactcagttgggtgttaaattcttgtccctgctttacttgaaac
tagtaatcttgagaaatttactaaatcattttgagccttgattaccttgcctgcaaagtg
aggaacattttcaatttaaaattctatttcttttattagtattcttcctgaccttgaatc
atttctattcccatcatgtccagttgccaagaaaaccatgcaacttaatgttgtttgtta
gccatcagattttattttactgaactcaagttcagtcacatcagttaccgataaaataat
aagttctatattaatatttgatatatcttgacattttgtattatggtaggaagaaataaa
agaccttcctcaaattttaggatatgggcttctgtttgttgatgtttgtgttttatgata
cactgttgtaaatcatactaaaggttttctctgaaaaagactagaattgacaatacttca
tttgttagcagtttcaagttttagatcacgggtgcataatttcttgtgtgtgcacaacat
gttggcattttatctgacttcaaagatgtccatatagataccagcaaaggataaaatgta
agaatgaatatcatgaaaagatgctgtatcagataaaattctagagcctaggttaaaaaa
cataaaggcaaacatgcttgatatataaaaattgagaaatatgataaaaagaagtaagtt
taaagcatcagtaataccactgcctaagatgcagtttattttgaaatgttgtcttctagt
cttcatacagtttaaaaatcatactataatcacaatttccagcattttccccttagcata
atatattataacatccatgtttttaaaaatattttatgaacgtcatgtttagggattaca
ccatatatatcccatctcatagattcctttatattgaacatttggatttttatttacatt
tttatttacatttatttttatctttattttttgctttcgtggataaactgcagtgagcat
cctctgtataaattttttttttctgttttaggattacttccttaggatagattgccagaa
gtggagttactgggtcagagggtatgaacttatgaactttttttttaaataaattttttg
ttcattttctgtccagtcatttgtaaaaaaattaaaataataatgattataattgttacc
tgctagaaaactgtgtctgaaggcaagattctgtgatcagaaaagtttgggaaataccca
gggtaaaaagccttttaatctgatttacctcagcatttcccaaattaactgaacactgaa
aattttttttgatacacttcttaaaattttgtggaagttttatataggatatagtctatg
aactactgatgtaaaattaaaattgttttatttgcttgaaatattatgttttattggtca
aaggaattaaactataaatcttcatgctaaaaattattaaaagggattagtgttgtttac
tatggtttttgtgcatgtctttataaagtcttataacttttaaaattgaaatgttctata
ttgtttactaaagaacattttaatgccttggtggcatcaatacttttttgcaatcattaa
tgatatttagaaagtagacatataacatgaaagtagaacataatatagattgtacaaatc
ttgttttttaccctattttcccctgcagaatgtgaaaacctttgcatcttctgatagtct
agccaaggtccaagaagtagcaagctggcttttggaaatgaatcaggaactgctctctgt
gggcagcaaaagacgacgaactggaggctctctgagaggtaacccttcctcaagccaggt
agatgaagaacagatgaatcgtgtggtagaggaggaacagcaacagcaactcagacaaca
agaggaggagcacactgcaaggaatggtgaagttgttggagtagaacctagacctggagg
ccaaaatgattcccagcaaggacagttggaagaaaacaataatagatttatttcggtaga
tgaggactcctcaggaaaccaagaagaacaagaggaagatgaagaacatgctggtgaaca
agatgaggaggatgaggaggaggaggagatggaccaggagagtgacgattttgatcagtc
tgatgatagtagcagagaagatgaacatacacatactaacagtgtcacgaactccagtag
tattgtggacctgcccgttcaccaactctcctccccattctatacaaaaacaacaaaagt
gagtatattcaatatattgttaacctgagaaactttacatatctattttaattgtaatga
aactttcctcacagtctttgtattactaaaaattaatcttaagatgtgtaataataaaat
gaactagttttttgtcttacagaaggtaatggaaagtattacaagtagaaatactgataa
tgggacaatctgagtacaaatggagggaaagtaattgagcctattactatgaaaaattat
taacatcaaattttcgtctaaaccttgcgtcccgggcagggattagtagcttgtgtgggg
tgtagaggtttcgggtgaaacatgatttgtgtaaatcaagtaatgaagaaactttttatt
tacaaaatgcagctgctagagtgggctattcacaagtgtcactctaaggcataaattact
cagaagttgcctcaaattagttctagttatttatcatttgagaattagaaataatgaata
ttcatgtagagtagacttacaagttaaactatgttttggaaaggaacattttaacttgaa
tatacttgttttatagacattaacataatgtctgtggtagttcatcctcttactcctact
tgcttatttcttacattttacagtgattttaggccataaacaatatacaaactgatttaa
catcgtatcgtttaacctaacttgacttggaatgaagtatcttacacacttggattaata
ataattgaaatgctaaatcatgttttacatctgcttgtaaaaccttactttctaaaatgt
gtttggccctaatttacctagcctatctgcaagtcagattttatttagctggcatgatct
aagaaggacgtgacatcaagtagtgatttttcatccattgtaccatccctgtctcaagat
gttgaagcagtagctttggatctcagccagatggctctaccactaacaactatagcacca
ctttgttaaggctcagaccttttatagtgtcattcctcccttttgcccccctagtcttga
tcatcttcttcaaggttgctatatcaaaagggagttttaacatctctttttttcctgagg
gttcagttagaggaataggggtttaatacttaaggtccaattgcactaacttatttcaga
aagaggttcaatcaaaactaagccttgcggaacaatgcagagttattgctgatttctgct
acactttaaattaaaagcaaaaaaagctagttagacaaaacattatctttttccaaaagg
aaaagtcatccttatattctgtaggaaaccttccagttcacaagtttgacaggtgcttta
tcttttctttaccttcaagatgtaaaattttgtgtatgatgattacctttttaaaaacga
agtatactgaatttgttagtttttttctagtgattctgaatcatgaataacaattattgt
cctgtacatataacaagaatggactgggctctaaccatagaaggaatacatgtgtgtatg
catttatgtatatacacatgtacagtatagatgtgtgtatgcatttatgtatatacacat
gtacagtatagatgtgtgtatgcatttattatataagtgtgtatgtatagtttttgatat
attgttgatcatgagatgatctgccttcgtaagtatggcatgaagaatacaaagctattt
gattgccaggacagtgatacccttctacaagtaagatttcagtctgaaatttttctaaat
gttttcctttttgtccattcctgaaacccttctgccagagtattcccagagcactttcaa
cagcattaaaaaaagtcactgagtactgcttcacctaaaaagaaaatgtcagtattctcc
gtgtatacatgttttaatacaataaatagaagctctcactagttgaagtactttattttg
gaagagctttaaattcttgatcattataaagttaagacacttctacagatgttaccctag
accatagtttcattcaagtcttctttgacttttgagagtagaggcataagaaatgaaatc
ccattttaaatgttaacataggattagctttctacaaattttactcaggatacctttcaa
gttttattttttggcattctcataaacaaaccctgtggacagctagactgaattttaccc
atcatccctaaatatgctgcataagcaaatatattaatgagaaaaaaagatttttaattc
ttccttgcattgctttattctttgaaagcattctgaaaactgtcaacactctgaggacag
tttttccccccttgtcactaaaggtttgttcttttgggagccttatggcaagcaatatat
gttaaaaattccatgttgtcttaatgttaaaagtcaagggaagtggtagtcaagaattgc
tatttattaagctcacactgtttgaaagatcctactacagatacataaacaaaagttaca
gagaggccgaatgatccaccccagattacactacgtgatgagagattttaatcctgattt
gcctaatagcaaaaaacccctttctctttccacatgctatgctgccccctatggttgaat
ttgacaaacatcactgaacaagatttccaacatagtcagagactctgccatgtgatgatt
ttctgatactatgtcccatttgaagtgataagagctaccctatagaaaggacagagggct
cagttaactacttttctttgtggcacttaacaaaacattattgaagattttacagctcaa
ggctaaagtaatcaggaacaaatgacgctgttaaaaaattttatagaggtcatactctct
tgaatccataaaacatgcttttaaagcagaggttgtgtgctggctattcctctaaatctt
gcttctccagatacaaatgtctgtctcttcagttttaactctcattctcaacatccttat
catatttttttaaaaaaagttgttttctgaagatatgcccagctcatattgaagaatctt
tgacatattttatgcagaatatcaactaattttctactaacatgaaaaacaaatgagatg
attattaggccagatctggtatcctgatcttttgatgaagagaagtgtgtttgagcatta
atttctgatgcttccatatctatcttagtggcttccacctgttctcttctcacataactt
ccttacctcaaattagtctatttttttcccatcttgattgttagattcttcctagtaatt
aaatcgtttttatccatttgcctcaaataagaaataaatgtttatcaaaaaataaatata
tataaaagatagccaggtgtggtggctcacacctgtgatcccagcactttgagaggctga
ggtgggcagattgcttgagaccagcctgggcaacatggcaaaaccctgtctctacaaaaa
atacaaaaattagctgagtgtgatggcatgcgcctgtggttccagctactttgggggctg
aggttggaggatcatttgggcccaggaggttgaggctgcagtgatctgggccacagagca
agaccctgtttcagaataaataaatagagggtggggggaggtgtggagaagcatctagtg
tattggagtttagaggcgtaagaccctttcttgtctataaattctgaacaatagaggaat
caggtaataggatcattaaccaatcagggtatctggtccgccattctccaaccacctcct
aggaccagagctgctgagcagctttgtaggataatgtggctaacattggaaatataacag
ggctctaagttacactctgttgtagagttctagagaatgatagcttcttcagaaagtccc
tggcctagtctacctgcaagacaagcaacttaagttcaaagtctgtttggtatatacatg
tccagagagcaaaaaatgctatttaaggcagtcatcttgaataacatccactagtaaaca
tggcccaatgaatagccatgtcccctacctttgtgaaggaggaaaaatgaaacattgact
cagcctaatttagcccaattttaaacatgtggggaggataatggaaatgggagaaatggg
gacaatgggtgagatgggttttcccctaacatattagagtgataagaacttttcacatct
gagtttgaattctgattataccacttactgtataaccatgagtatataatctcagtgaac
ctattactttatctgtaaaataaagactcatgcctacctctcaaggtttttgtaaggatt
aagtgaaataatttatgtaaaacaagtagcatagtgcctgggacactcagatattttgca
gatatcggccttgtgacctcattttttccgtctcctgtgccttaccccaaatcccacaaa
ataaacttcattccccgcagcggttgtcaatctctatatatatgttttacaaccttcagc
attcagtcatcatatcaccctctagtgtaaaaagtagagaaataaattcttttaaaaagg
tacataggaggattagaagagtactgtaataagctaggcccatcttcaataaaaatagac
ataaattttgaagtcgatgctacccacagatctccctagttgataaaatatgtacccttt
cctttcattagaatttaagtcaatttctctagactttcttctaatctttaactaaagctt
ctgtatatttaaatgctttaatgaatcttccatatggatgccaaaatgaattttagctca
tggttaatttctaattaatgcctgttaatatagttttaaatgtatttgttaatattattt
taaacaataatagctctaaatgttggtgataaactgtatctcattcactgcaggtaatga
cagttactattattaattatttaagagacggggccttgctgtgtttcccaggctgtcctt
gaacccctgggcataagagatcctcctgtgtagctgggactacaggcgtggaccaccgtg
cccagctcaaaaagtattttaattatgaggtttcattgtagactaaagaataatcttcac
accaaatggaccattttaaaattgttttatagcattccattgattagttctttatacaga
tgataaacttcactgaagtatacacaaatatcacaaatagttatcttcaaaatgcttcca
aagaaaattgtttcatatgtgtattataatgatctgatccatttttaatgttgtttttgt
tggtggtagggaagacagtatgaagaaaaaggacataggaatagatcatgatgaatattg
ttttcactttggtagttaactccaaaattatttctcaagctccgatcattcaccttagcc
atagttttgcatttctagctgtctgttgggcatcatcatctgagtggtccactgaaaact
aaaagtactgtttctttctccaaaccaagtttttatttgcctccccaggttatcttttcc
ctctcaataggtcccatcttctggtcagtggatagaaaagtggcttgactttttcttatc
atctgagtctgggcctgctgaataatttaacagacttgatcacttagtctctctttttct
tacccgccaggcaattgtaagataaaaattggagggtcagtgtttaaaaacgttaagagc
atttttgcttgggcttctgtttgcatcaggctataacgccacgtccttgacaatgagttg
gcagtatgaataattggatgagttttttaaaatgaatttttagctctgtatagataataa
cctcaatacagatatctggtgccccatttcccactgataaagccaaacctaaatgagaat
gtttgaagtcttccaatatcttaccatagtttttacctggtttcatctcctctcctctct
acttgttttgaatcatactccttcccaccagccatatccttcaaatcgaagtccaaatcc
cctatcttaataccttcctagatttagaaaatcgaattcctttcaacctgttcttgcttc
tcttcatggtgcagttcatttggtattacctggtgacatttttggtaagctttgtcttaa
gttgacatataattcttcacttttttgagcagtagaaatttggtaaatttgttttcagtc
ctaattcagtttgagaattgaagactggcaaactgtaatgtgaacagttgtcatttcacc
tctttttttttttttttttttttttaagacaaggtcttgctttgttgcccaggctggatg
gagtgcagtggcataatcatggctcactgcagcctccacctcccaggcttaagcaattct
cccacctcagcctcccgagtaactgggaccactcgtggcaccaccacgcttggctaattt
ttgaattatctgtaaagacaaggtcttactatgttgcccaccctggtctcaaactcctga
gctctggtgatcttcctgcctgggcctcccaaagtgctgggattacaggtatgagccacc
atgccccagcctcatttcacctcttaagatgtacctaagctagactagaaattgtaaatg
ggcacgtcattaaaattgagtttaagactcaggtattcagtatatgtacagatcgttaaa
ttgagactgccctgaggagaagttaaaaatcccactttaggaatcattttgctaacatgc
agaaatcagttgaaggcaattcatacagccattaaacatcacatttgaaagaatatttaa
taacaggaaatgcttaaaaatattttatttaaaatataggattcaaaactctatacagtg
tgatcttaattatgaagcaaacatacatttttatatatgtgtgtgtgtatgtatctacat
atatacatatgcaaagaaaaagatcagaagaagttgtaccaaatgttaggaattatctct
gaatgtggttgaataaaaggttattttttcttaaatacattttataatttctccattttg
ttataacaagcatatatgcctctttatttgaaaaataactctctgctggaaaccaattat
tattctatgtggtcttttttgatgactttacatggtgtataatgctcctaatggaggatt
ggataaaaagatgttcctcacaggaaatgtcattatatcatgatctctgcatgagaatgc
agctgaatatagtggaaaccttcccctattcccaaccccccatttcatagtggaaagagc
acagaactctttaagtcctagtgtaactgattcttcacatctctagggctcaggtgttac
cctggcaattttctaagatcaacttaacacgttattagaagagtaatagcatagtagtat
ccgttcttgttttgaagatatttattgttttctttgaagagttctttagccttgatagca
cttttggctctctctaaacatgaatatttttcatataaaaaccgttctccgagcaatttc
agagtctgtttttgaaaatgggtgtccccaacccccaggccatggaccagtctgtggcct
gttaggaactgggcctcacagcaggaggcgggcggtaggtgagtgagcattaccgcctgt
gctccgcctcctgtcagatcagctgtggcattagattctaataggagcacgaaccctatt
gtgaactgtgcttgcgagagatctaggttgtatgctccttccgagagtctaatgcgtggt
gagctgaggtggaacagtttcatcccaaaccatccccccacttcccctgtccatggaaaa
attgtctttcatgaaacctgtctctggtgccaaaaaggttggtgactaccgttttaaaac
acaattcttttttttttttttttgagatgaagtctggctctgttgcccaggctggagtgc
agtggtgcaatctcagctcactgcagcctccacctcccaggttcaagcgattatcctgcc
tcagcctcctgagtagctgggattacaggtgtttgccaccacgtccagctaatttttgta
tttttagtagagacagggtttcaccatgttggccaggctggcctcgaactcctgacctca
agtgatccacctgccttggcctctcaaagtgctgggattacaggcatgagccatcgtgcc
cagccttaaaacataattctaaagtggccactgctttatgaggaaatcatcagtgctttc
ccttccctcccttcccgtattcccaactccccatttcatcaaactaatgatatatttaac
atgttaagggtttttagtcctgattttaagtctcctaagacagaccccaaattggagtgg
acctttgagatcaccaaggccaaacctgtgcccagtggctttaccaacagacttgaactt
acaaatgaggtcattacaaaggaatactgcaatgtgaagtatcttcatagttttagacgt
tatttatgttagggttctttattttgtgttgacatctctgactttgctttggtccttgtt
cttctacctagagcagtgacagctcttttcataggagaagattgctgccatattcatctg
tgatcttctccagactgaatgccttcaatgtgttcccagaatgtgtatatatacttatat
tttttctcatttatcttaacagtcctaatatgtcatttctaatctttaaccccataatgg
agatgagaaatatgtgaagagaggttaagtaattaatcttgtatcagtcagctggtaaat
agtggagttgggattggaactcagatcttatgctgggtcccggttgtacttttgttaacg
tttggaaattcagttaaacaatattagtaattttgggatgagtgggctgtgatgaaataa
cctataaagctaccgccctaacatttattttcatgtccccaaatgttgcctcaactaaat
ctctgttactgtggtatatttattgtgactcatcgtattaagtttgtttttctccctctc
tcttaagccctttgatttcttattttattgcctaataatatttattgtgttatcaggtac
cctgataacctaattattagtcaacaaagaattagtgtataaatttttacataatacttg
tcagtcattgaccacatggatatgtattttgtgtagctggagtcagtgtgtatacttctt
ttatattcctgattctttcagttattatgataataatagtcttattattatttggtaacc
tgtttgaacagttgacttgttcagcccctcaaagggaatttcccccactcccctctgctt
cactgaaacatgacccattaacatgaattatttctacattccttcacctagtatttcata
tgtatcttttttcttctatccctccaatctggtaagaagaaatacacttgtcctttacaa
ggaaaccttgtctttgaattattgatccttgaggtctgcgaaatgttttttggagacttc
ctgcattagccattctgtctttttaaattaacaagctttaatttttagattagttttaga
ttcacagcaaagttgaatgagaggcctagagagttcccatatgccccctggtgcacacat
gtacagcttcacccttaccagagtagtacatttgttatagtcagtgaatctacctttgtc
atatcattatccagaatccatagtttccaccaaggtctcactcttggtgttgtgcactct
gtgggtgtgggcaaatgtgtattgacacgcatctaccaatgtagtaccatagagaatggt
ttcactaccttaaaagtcctgtgttctgcctattcatccctttcttccccagcctctcat
cgccactgatcctttcaccgtctccatagttttgctttttccagaatgtcatatagttgg
aattatacagtatgtagcctttcagattggcttctttcacttacgtaatatgcattgact
tttccttcatgtatatttatagcttgatagctcatttccttctagcactgaataatattc
cattgtttggatataccacagtttatccacttccctagtgaaagacatcttggttgcttc
cacataattggcaattatgaatatagctgcttgtgaaaatctgtgtgcaggattttgcat
ggactaagtttttaactcctttgcgtaaatgccaaggagtgtgattccaggattgcaagt
taacagtatgattaattttgtaagaaactgccaaatggcttccaaagtgtctatatactt
ttgcattcccaccaacagtgaatgagagttcctgtggctccacatcttcagtggcatata
gtgttgtcagtgttctggattttgaccattctgataggtgtgtatggtatctctttgtta
ccatttgaagttccctagtgatatatgatgttgaccatcttttcctgtgcttacttccca
tctgtaaatctcctttggtgaggtgtctgtccagatcttttgtccatgttttagtcaggt
tgttctgttactgttgtatattttggataacaatcgtttatcccatgtgcattttgcaaa
tactttcttccagtctgtggcttatgttcttattttcctgacagtgtctttcagagagct
gacattcctattttagtagagtccagcttatgaattatttctttcatgtatcatgctgtt
agtattgtattgttgtatctaaaatgtcatttccggctgggtgccatggctcacacctat
aatcccagcactttggcaggctgaggtgggaggatcgcttgagcccaggagtttgagacc
agcctgggcaacatactgagacctcctccttaaaaaaaaaaaatgcacaaaattagccag
gcatggtggtgcgttcctgtgatcttagcaacccgagaggctgaggttggaggattgctt
gagcctgggacatcaaggatgtggtgagccaagatcataccactctaccccagcaaaaag
tcatgtctatacccaaggccatttaggttttctcttatgttatcttctaagagttttata
gttctgcactttacatttatgtctatgatccattttgagttaattttgtgaaggttgtaa
ggcctttgtttagattcatttatttgcatgtggatgttcggtggttcagcaccatttgcg
gaaaagactagcttttctacattgtactgcctttgttccattgtcaaagatcactgtacc
atattctggtctctctattctgttccattcatctacttttaattctttcaccaataccac
actgtcttggttactgtagctttatagtaagccttgaagttggagagtgtctatctccca
gcttcattcctctccttcagtatcgttttggctattctgggtcttttgcctctccatata
agttttagaatcagttcgttgatatctgttacttgctgggattttgattggtattgcttt
gaaactatagatgagattggcaaggactgacatcttgacagtattgagtcttcctgtgca
agaacatggaatatatctccatttatttagttcttctttgattttttatcagagttttat
agttttcattatgtagatcttatacatattttattagattcatccctaaatattttattt
cagggggctgctaatgtaaatgatactgtatttgtcatttcagatttcactttttcattg
ctggtgtgtaggaaaataattgacttattatatattaatcttatatcttgcaaccttgct
ataattgcttttttttggtcaattttttaggattttctatgtagagggtcatgtcatgtg
ccaaaaaagacagtgttatttcctctttcttaatctctttatcttttacttccttttctt
gtctttgtgctttaactaggacttcccctatgatgttgaaaagcagtggttaaagaggac
attcttgccttgtgtctgatcttagtaggacagctttgagtttccatccttaagtgtgat
gttagctgtaggctttttataggtagtctttatcaagttgagaaagttcctcccctctat
tcctagtttgctaagagtccttctatcctatttcttaatggtttagtagatgactctgtg
gtactttgaaggttgtttgcagaatttccatgccataggcaatttacctttccttgacat
ttgaaggattgatgttggtgccaagtatagaatcttcacagagtcctcctgtagcttcta
aaggtttagcttgaaaatgttaattgcttaacgctagtaagtgagtgaaaaagctgggga
taaattttgtatcttgcttatatttcagttcccacctctgtcctgacaaaaccccaatat
ataaacagtaaatgtaagatatatatattttaaacataataacaaatgtaagttcctttt
gaaattattatttcaccaactttaaacagtattatcaatttttcaaacatttggccagtg
gttacaattttatcaatcttattagtgagattcttttctgcttgtgggtctacttttact
ttgactcttgtcctgaaatctcaattctatctttggcctctcttctgagagacataattt
tgtgtatttaattaatctctattttctataaattaatatgataaaaagaatcaagttgga
tggatcagaagtagaatacagagaaaagaaaatatggttagggaattgtgattaggtatg
tttaaatacttagttcgtagacacgaccccattcttttgttttttctttgaaatttgcct
tagattcagtattgtctagttcttaaaatgctattttttttcatttagcattacaattat
ttgtgggaaaaattttaagaacatttcagaattctttcactggatattctaatatggata
gtaattcaaaagaaagcacaaagatgtaaaatcacaaaagatataaaatatttaggtttt
tgtaaaaatcatatattagtatgtttagcatacatagaaaattttataaagtattatttt
tttttttactgtgaaacatcatatcaaaagcacacaattccttaagaatttatctgtttc
ttcactgaacactagagggccatatagcaccttatagatagagcggctttccatatattt
aaatatatttaatatgtgcttgtctttctctcctactttgtgttttagtatagtatgtct
tgctcagaatttggagtatacactcaacagtaattatgtgcactgtcagataatatgtgg
gatacctagggtttttgtttttgctttttttttaacaagcagctagcattttgctactta
gtaacaacaaaaagagataaagagtgtatcttgggataaatgtatgtgcagacttactat
gctttggaagtataagtatgcttttagaaaatttctcaagtatctgaacatatacgtgta
aaaagaataggcctctcattgttatttgctctggtacttcttggcatgtatgtgagaaaa
gaattatctctccaaagaagctacttcccttgtctaaatgtcatcttgtattagcacaga
cagtaatattgtggggtataaccacctaatcatcgtggagtaaagaatatttaaacaact
gatttaatcttcctcaattttttttttttttttttttttttttttggcttcttagggtaa
agaaatgagaaatacaacttacaggaacttaaataggtaatgtcaatgtgtgaagagagt
catgaacagccagtaagactgttcaggctcttatgaactacgtaagatatgccttcccca
aaaggtgttaatgttcagttaaaagaataaaaatatgaggcaagaggatggtttgagccc
aggagtttgaggctgcagtgatagtactacagtactctagcctcggtgacagagtgagac
tctgtctttagaaatatcaagaaaagaaaaagaaaaacattgtgctggctccaaaaaact
gactttgagctacatatgacctatgggctgtatactgttggatctttaatagcttctaga
gaagaagttcaagatctaagtagggccttggctaaattttaatttcttccttagatatac
tttagacagcacttttcattattaatttctgtgttatgtttcagggggcagaaataagga
catttctcgttaactactgtttacactgtcttttggtgaaggaagattagtatgatgaac
ttagaggaaacacactgtttacacctagaggagaaaataaaagagggtcatgtgaaagag
aaagaaggaacaataattcactcttaaagacttaaaagaatctcatcttttaagtctctc
ctcctttgatttgccaattttaataccttaaattatttcttcaaattagttactataatg
gagtgacaaaactttgatctttagttctaaattttaccgagatgttagtcctcttgaata
ttgatgaacttacaattattttaaagtcactgctatgataaacagtattctctaactgat
aatattttttatgccttagtggtgtgtttgctagttttctgtatcactgccattttctga
aaaccacatttattggcagattgacatgagcttaggtaattgaaggctgagcttgaattc
tgatccttttgcacaggtaatgaaatgagttattgttagcataatatagctggtgcatgt
tgaactcttaaatggtcaagaaggtaaatcaaataaaaagatcagtatttggatatgttg
aaaaacacatggactccttctttgagtggcagagataatagatgctattgatggattgaa
gacatccattattgtgtctttaacacgtgtctggagaaaacactggcttgctagttaaaa
cttgattcatttaacagtattgctgagcactcaaagaattgccattatttttctaagata
tgccacaatttaatagtagctcttgtagggaggggttggggaaggacgatgttgggagta
gattactatgctaaatgcatatattcattgttagttgcattacattttaaaagggttaat
gggagggggcaagtgcagaattgaggatatgtattattttataaagggtaagagatcact
tttatccacccagaagtcagaggtttggaaaaagcaggttagtatgaatatgaatactag
agatgtttctagaatatcatacacctgtattgtaactaatttccatcaatgatttattta
ctaacctggaatgattttcaagcagaaaaccctctggaccatagggtccagaaagttaat
tgatatgaatgaaatggactaaatataagagccagggagctaaaacttctctaaaaagag
ggtcttgatattgccatgtttttgaatttttcaaaattcattggaatttcagattttcag
atgatatttcttaatctttaaaatatattggtgtccagattttgcaaaagcacttccgtg
ggttggtttgacccatagaccacctatttttcactagacatcatgtagggaagaagaatg
gactattattcttgaactgaataactgaaactgaaataaatgcaagggaagatgttcttt
ggcacttctgtagaaatgtttatcacccattgcaaaattacgatagtttgcctccagtgc
tgatccattaatgggaattgggttgtttggagtggaagtgcggcatatgcatagcacttt
aagtgctttagccagtgacctacggacttttccttatgagtttggtgtactgattttagt
tattaaactgccaaatgaattgacctcattttaaagaaatgagtatccaacttccacaag
gaatttgagatttgaagtggtctatatgtaggaaaaatacatataataaagtgacaaagt
gtacatgaaaatcagaaccagggacaatactggagtagaatatgagggcaaggtcatgag
aagaggtaaggattgagttatgatgagttggagaaagctagtaataggatataggttgtc
cactcttaagtgtctgcaccagtatttttcaaagtgttaccttggaccagctgcatcaga
agcatctggagtgctttttaaaaataacagatttctgggttactctcaagagctacttaa
ctaaactccagggtcgggatgtgcatttttaataagttctcaactgattttcatgtaaac
tgaagtttgcaaaacactggtgtgtagtttgttgtctctgctgccctgtgataaaattga
cgatgtactgagtcctcaggggaagagaaaatatgtcacacacacacacttatgcacacc
catgcatctttcctaatattacataaaaggaatttcccacataggacttcctgagtgact
cagtagatcatgtcctcaacagtatagctataacaaatgcattaattctcatgaatagtg
gtacagtgtgatctctaaggtcaaactacggggggattttcaaatgctagctctgtcact
tcatggcagtttgaccttgcacaaaccgttaaccttgtagagtttcttagggacggtagt
agcatctaccacatagtgttattgagaggattcagtgaggtaatgtatgcaaagtactca
ggacaatgactggcatatagcaaatccttaatgagcatttgctatggtatattataaggt
taattattgtaatgttacaagtctatgctataactaactcactagttcaggctgggagga
atgctatgcgaaatatttttagttccaaagatgctcttttgggtaggttgaaacaattcc
attgacttttgcctacattattaatggcttttagcatatagttcagctgtttgatattgc
acattttgttactgctaactatgaagcactcttgatccccgacagatattttttccctaa
ttggttgttaatgctgaacttagaaggaatttgtaaaagaacctacaagtcaattttggt
aatcttagagctttgagtactcatatgaaaatgttcagaagagaggaaaaggcaagtttt
attctttaatgatacttgggtgttcttgaactgaatagctgaaactggaataaatgcaag
ggaagatgttctttggcacttctgtagaaattctagaattactgcttgatactacagaaa
atatctaagtttttcacagtgaagagatgattataaacaaatgaagcaatcactatgaat
ggaacaatagttttgggtaggttaaaattatctttaggatacattttagagaacacttcg
taaattattagggtaacttctagctgtatctgacataggatattaacttcattccttgta
ataagtattgtcagggtagcatcttgcatataaactttaacctctaattatatgaaatgc
aggaagaatgatgattgttttcatagatcaggtttaaaaatgatgtttaaaatttttttg
tatctttccccctccccagaaaagggtctgttttattcaaacgtaccttgtccttctgtc
agttggtttttgctgatgcaaaaggtagtttctctgctttagaaatagcagttgctttta
ggagatgcattacttggttggtttccatggcagtttttggttattgaaatcagatgcaaa
tgaggaatggaagtatgaagaaggtactggtgaatttgaggagtaagtgccaacagttat
tcatttgcttagctacagatgttggttctattaatataagactacattataagtttaatt
ttctagaatgaattgtttgtactttaccgatatgctgccatctgtcttagggtttaatga
gtgaaaaacttaaatatactgaggtacctctgtagtgtttatcacgtgtttcagttttgg
ggtttattcatcattatcattttactctgttacttggtatagaaaagaaaattaaaactt
taagacagaatcattattctctatcagttagattatcactaatcttacattttattttaa
ttatatttttgtgcttctttactcagaagggaacagaatagagattacaaaatggtgcct
tgtaggtatttaaatctttttcttttcatcatgacagcttaccattgaatgtatttttaa
aacacatttaaatttgttgccaacattggaaaatcttgagagttcacataaaaatattga
tttttttcaccttcttttgaaaatatggaacaaatggctttgctgggcacatgtacttac
atggcaacatcaactgcaccttctaaaagggcttttcagatcttaaacctaatttgtttt
tctcctttattatacttgccttatttgtcatctgaatttgcttggaatatagacaaaagc
atgattttaggaaatccaaaagtgagtttgatttttcctttcttggtcataacatcaaat
gactttagatgtaaattcaagccaaagtaactctttaaaaagttttattatgaaaataga
aacttgctaaggcctcatttccattgagtgtcaaggtgggaaattgtcacccaaaatttt
gccatcctcaagatgatcacttaacgttttcattttgaatcacctgttgtcctttccatt
cctgttacattgtaaaattaacactccactaggatgactataaaccaacttagagctcct
tgttttataagatgtctcggttatccattgtcacctgaccaatcaccgtcaaagctttgt
ggattataacaaacgagaattttattatttctcattgttcttataggtttgtaatttgag
gagggctgatctggccattgttaactctgggtggcagtcagatcatggctggaggtgcaa
aagtgggggtctagaacagctgagggctgacccagaaattctttttccgtatcatctcct
aacatctctatataatacggtctttttacctgggctagtttgggcttcttcagagcatgg
tggcctgaaggtactacttagccagcacatcaggactaccaagtgagtgttccagcaagc
aaggtggaagctgaatctcctttttatgactcagcctgtgaagtcacatgataccatttt
tactatctctaatggtccaaacagtcacaaaggtctggcctgttattaaggcaagaggac
atggcctctccctcttagtgaaaggagtctcagagtcaccttgcaagaagagcatgcaag
atgggggacagcgttgtgactacctttgagaatacaatttgctataagagttcataatat
ctttgatgttattcctgtttctttttttaaatttgggcttcataaaaatcaggcacatta
aaacattccatatttaatatgtaaatgtcttatacaatactttatttcagttatctagaa
ctcatttatatctgacaaacttacatatctgaaacactgacaaagaagtgtctccgaaca
actaatttaggaagtaacacaactaagttaggatgtaacattctgtggaattggatagat
gttctgttacaaagcacaggtttaggaattagacataccagggttaaaatctcaactcta
ctacctggcagctgtgttaccttgggcaaatttgtaagcctttctgagccttagttttgt
ctgtaacaagtagacaaggataatgatacctcagggtttcggcgtggtgattaagtgata
caaagcatgatctagtagaaactcttgtcagtgtttaatctcttccccttatcattgttc
cagtctgaaacaatgcgtacgtattttctaagccctatagctgcagacttcctttatatc
tcttactgatctctgatagtctccttagacttatttatctttctatcccaagatctttgt
attacaggcatactaaagaatagtaggaattgaattttatttctttgtttctaatttttc
ttgatagccagttagtgttttacttactgtaaagaatgaaaacttaggctctcagcttaa
tttaaacctcagctacaaaaaataacattgatatttttcagaaaaattatttttttgagt
atgtaatattgacagcgtaaaaggccagtggtatcaactgtcagcccgtccaagaattaa
gttctaaatatgtattttctattgcttaatcttatacttgttttctgtactttaattcta
tacttaaattctgtgctgaagtactttcagttaatattaataaatctcacatacttgcat
agcacattactgaaaattatataaatgaagtatggtactatgtttatctttctcaaaata
tattgatttttatttataattgcatcagaaactcaacagtataggaaataatgtccccct
cctattttacctatttaaagcaccagcactccttcagattttttatgtttccataagtgt
agctttttatttttcataagataaatttttataatttgtcaaactgtcaatttggttgag
ccttctgcattatagcttaattttttaaagctgtgtagcaaaaaaacacacaattactta
ctgtattttaaaagaatattagtattatttccttagtcctaattattagtataattagta
ttatgactccctggcctgcaatactcttaagaatttttataagagaaagatattattggt
aagtaatgtttaatttattttaactgaaagtttaaactgttatgtttaaactgtttatgg
acttaagtactgacattaaagaagttattgtctttcttagacttaaattttattttgaga
attatggaaaattaggttactttaaatcacttactctgtcattacatgaggctttttgca
tgactgaaatacctgtaattactgtgtcatttttgtcatagttgacttaaaggtgtaact
ataataatcacgaactaaattcttggtgagcagctgtgtaaggccattttaacagctggg
gtaacccagctcagcctgtaagagttaagcttaatcatgagacaggttatgttaagtcat
tgaagacttcatactgggcatttctgaaatacttagaaaattcagtgggatagttggatt
tttgttttatcttaagtactttttaaaatgcttaatgaaaggatgaggaaaagtatttta
acaactaaagttcttaggcacaaataatagaaataaaatccaaagtttactcattaaaaa
aaaaaagtcaaatatactctatatgctagtgtttagaccactacagtaggtaggagagga
aaaaaatatataaacacacacacatattattatagtataatatggaatcagaaaaatcct
gaggttatttttcttgaatttcatcttttaaaaatttttttaaaaatcattatgcctgta
gcttctttccaataaaaatgttttcttattactatacaaaaacatttagagcacaggcat
acatgccttttgtaaaagaaggcagacacaaaagaattgaaacaatgcattttatttgga
tatattttataaactagctatattgttcactaaactataataaaggaaagctcaccaaat
tgaaccttcttacaagaaggccatatatgtaatattattagagctgtaatatgctttttt
ttggtataaatttaaggggtactctagttttttttacatggatatattacatagtggtga
aatttgggcctttagtgtacctgtcacccaaatagtatacattgtacccgttaagtgatt
tctcatctctcagcatcctccctgcatgtcacccttctgagtctcttaatatctattact
acacactccatgttcatgtgtacactttacttagctttcacttgtaagtgaaacgtggta
tttgactttctgtttctgtgttgtttcactgaagaatcacctccaattccatccatgttg
ctgcaaaagacatttcattcttttttatagctgaatagtattccatttgtgtgtgtgtgt
aaacatatatatatgtgggttagttatcatagttgacttaaagttataactattataatc
accaactaaattcttgggcatctttaaaataatgatttcttttcttttgggtagatgccc
agtagtgggattgctggatcaaatgatagttctattattagttctctgagaaatctccat
aatgttttccatagagattgtactaatttgcattcccaccaacaccgtataagcatttct
ttttctccacatcctcgccaacatctattattttttgtctttttaataatagccattctg
aatggtgtgagatgagatctcattgtggttttactttgcatttctttgataatgatgttg
aacattttttcatgcttgtaggcatgaaaaaatatgtatgtcttcttttgagaagtattt
attcatgttcttttcccactttttaatgaagttaatttggtttttgttgttgagttgcct
gtcaattctgcatatcagccccctgttagaagcatagtttgcaaatattttctcccattc
tgtaggttgtcttttcattctgttgatttagagatgtaatgtactcttaaacctgcaagg
gacttaatctcaatcatctgaagagaagtcagatagtctatatctattaaatatttagcc
tcctctttgtagaaatctgaagtataaactccaggtaattgtactgcaagacattaaata
gttaaggttcctgaatcttggagtttctggtgtaccatgggatggttttgtcatctgtca
acttataatttattagcacttgttttggttggttttctgtgttttactttttaatttata
atgtattttcagcaaaatttttggaaataactggaatataaaacagcttaaatgtctaac
attgagatgttgattccataaatcagagcagttacaataccttatataatctagtattct
ggcatttttattttgagaatgctacctatctgtgcatataggcatgtacatgcaatgctt
ttattttgcaaatgtccaattatcaggtcacatttttataacacttgtgtatgttgtatg
tgctgcttcagaacccaagcatatttctcttagttaggggccgcccttgttgcccaaatg
aagaaaattagcagggaagtgcagtatgttgtccattgaatgtttacatacatgtaatgt
ctcaaatacattataattggaagttgtaatctgagtgagcactttgagcatgtaaataaa
tatcttttagaacatttttagtaatcattttaaatgtgattttaatcttataaaaacatt
ttaatttatttgacataccttttgtgaatactaaagcttaaaaaaaaaatactaaaactt
taaaagatatcctgaccaggcgctgtggctcacgcctgtaatcccagcactttgggaggc
cgaggtgggcggatcacaaggtcaggagttcgagaccagcctggccaacatagtaaaacc
ctgtctctactaaaaataacaaaaattagctgggcgtggtggtgggcacctgtaatccca
gctgctcaggagactgaggcaggagaattgcttgaacctgggaggcggaggttgcagtga
gccgacattacaccactgcactccagcctaggcaacagagcaagactcagtctcaaaaaa
aaaaaaaaaaaaggtatgccatgttttttttctttgatgcccaatctaattgtatttatg
tttctgcatttaaagtaatatgttgcttgctggatgtttttttttttttttttacagttt
ttgctaatacaagcttgataatttatcattaagcagagggtttaatttattgatttatga
ttgttttcctctgtgtttaaattgttccgtcttcaaccatgaattgcgttcatttgtgaa
ttgcttggtagggaaatactttaatactctgatatctatgatgttaggaagactaaaaac
tgatttggtagataaaataatctgctttactctgtatctgtgaaataagtgagtatgctt
gataataccatattatctgatcagaaggattttgattctgatttattgcacttcagtaat
ggtatactttttagcatgtgggactttctgtttctacaacagagtaagtcacttgttgcc
tgaattcacagaagcagtgaataatactgaattcaaataacatacgttgatattattcct
gtgaaagtttttgaggtaagactttgaaaactcttggtataaagtcaatttgaatttgca
gggagatttacatcttctgagtgatttgttttagctgattcaggaataatttatgaaaaa
tggaaaatctttgtgctgctcatatgctgtggctttggtcaggagcagttaaatttctag
ccttttgtataatactgaatgaaagaaaagatggaaatccagagtaaattgctgtgatgg
tatttttattgttgttacaagagaattggagtgcttggacttgtaatgcattttacagat
accgtattggtgatattttattacatgtcacatttatatataattgtttttacggatgca
aatttttagaagaaagaaaatcagggagtgataagtgggcaaaataagttaccttatata
aagaccttttctttactacaagttccctaatgtttagcccatcttggatgatcagtggta
tctggaatcttaaaatgccaagacactatggctaagatactacagcttcatattttggca
aaaattttgacagaattatagaagtctttctgaaacttacaaataatatgttttaaaata
ttcttttccaaagttattatagatggattttagaatctgctttatttcctggtgatttta
aaattggaaatgtaatctacaaagctctatgtgatttttgattccattaatagtagtagt
aatcataatgacagtttaattaacactatcgaacacttactagatgtgaggcgttactct
gtatacttatatcaactaatttttaatcctcacaacagtcctatgagtactgcctaggtg
aacttgagacacgaattaaattaaggtcatacctggtaaagccaggatttggactaagtt
agtttggttctggagtccatgttcctacctatcatattgcactgccttattatttgtggt
atcatctttaaaagagcttgtctgtttttaagagtctcaatttgggtagtaattaaagag
tatctttaatgtttttaagagtagaattgagactcagactttatcttttcttgaacacat
ggaattagctacattttgaagtattaacagttggggttcttatataccttgtctatgatt
tccctcatttattagggtttttgtctatatttgaaggtttgtttttttagtgtttcgtgt
tgattgggaatatcgcattagaaatactgttacttgaatcctcccaaaaaaatgaaactt
gtagtatatatatttggaaattagtactcactttgcccaatcattgataatgtcttcttt
ttccaaaaatatattatgacaaacacaggattctgttacagcgtttggtttgccacagtt
gcaatgccctgtattatccagttgagggcagctatttactgatttaaaaaaataataatt
gaattagaatgttacccccttgtgcttaccaaaacttggtatccactaaaaaactccaca
tttgaggtacaaacagccttaattgtgttcacttatgttttgtttctgttgtcattgtaa
attatttcatgaaaatgaaaagcctagctttacttcatagtaacttttggtctcttagaa
actagaaactgttgattttagttttaattttcccaatccaagttcttttcaattctaaat
cttttttccccccgttaggatcagactcattgagatcagggattttcttatctttaaaat
gtctgcatgtaaatacatgtgactttagagttctttgagggcttttaaatgtaaaagtga
aaatttataatattgaatacattatctttattaaccagtggaataaccactgatggctaa
ctaggtctttgattcatttttctaaatgccaaatgaacatgttaaactcgtagaatatat
taccctcttctaatttttctgcattggagtatcacattgttaaaattgtatcttctatgt
agttcaggaaaagaaattcaggggggttaattatccaattgatttcaaaggtatcactga
gacagaatttttttgaaaataatctcagactgcagaaaactgttgtaatattaacaggta
gtgttagtaactgagataaggatattccatgggacctactttagcaaaagggattgtcta
atgagagaaggacaaaatatgacatatgtttcacaaacaaaacaattagtcttatgttgg
aaaataaagtgaacatctgaggagatgctaagactagcatgaacctagtattcattctgc
aggatagtaagttgtctcatagggctgtagacatggtatcttggatatcattctcacttt
ttttctactaccagtttataggtttccaacactctcatattatctcttctctctcctaac
cttagatctctcgaggtttttttggtgatgattattccattttcaatcatttactttttt
gtttaaaaacatttaaaatttatggttaaatatccataacatgaaatttaatattttaac
catgtttaagtatacaattcagtggcactaaatatatttacattgttatacaaccattac
cactatccatcttcagaatattttcattttctcctactgaaactctgtgccaactaaaca
ctaactctcatttccccctctccctgtctcagacaaccatgattctgttttctatctatg
caaatttaagtattccaagtaccttatataagtggatcatacactatccttttttgactg
acttattttacttagtatagtatcttcaaggttcatgcatgttgtagcatatgtcagaat
ttccttctttttcagagctgaataatgttcccttgtttgtatatgtcacattttgtttat
tcattcattcattgagacttgagttgcttctaccttttggctattgttaataaggctgct
ataagcatgtgtatacaggtatctgttcaagtctctgcttttaattatttaaggtatata
ctcagaagtggaaatgctggatcatgtgctgattttatgtttaaattgttgaggaactgc
catactattttctacagcagctacattcattcatttcacattgccgccaaggatgcacaa
gggttctgatttccccacatcctagcccatgcttgttgtttactgttttgttttgtttta
aaaccatcctaatggatgtgaagtggtagcgcattgtggctttgatttgcatttccctaa
ttagtgatgttgagcatcttttcatgtgcctattgaccatttacatatctttggagaaat
ctgtttttgtgtagcattgctgccagtgattttgcatttttaactacattttaaattgtg
gtataagagaaaggtctactaataggaatgggactatttcttgtgcatagattgccttcc
cagcagcccactttaatatttagcaatgctaatctttccaaagtcaaaaagaaatcaccc
atttctaactttctcctttcaaatcctttcttgtctccgtcaaaattcctgctataaaac
tccataaaagcttggaagttaaaagttggaaggagagataagtggagctgactggctgtt
ggaagaagaaaatagaatggaaagagagaatagttgattgagcactgtgaatggatagtt
cacaatcattatctactatctgttaggaatatggattatagtataaagattaattaacac
ttgcacaaatggttaatttagggaaatatttgagataatgctggaaagtttagtttgggg
ccactttgaagagagtcttcatctccagactttatcttaggccgtggagaaccattgaag
gttcttgaggagtaaacggcacactgcattccctctctgtgttttaggaagatttatctg
accatactgtgaaggagaaattaggcactagggatttgcaatggactggctagttactct
cccattttactataattacttcccatcatagattactttataaataaatacactttttac
atgtctttaatcagttgggtgactgttatgctttcaagtataatcaataagtttttcaga
attaaagattgacaattgaaaactctggtacaaagattaatctgaagtgtgtattaatag
ctattagagaaccagagtatacagtatttaatgtctagtgagtatgagataaagaatggt
gtgtatggaaaagggaaagagagcacacacttgaaaatagtgctcattataatatatcta
aggtagaacaaacatacatatgtgtgtgtgttttttttaaaccatacgtaaacacagttc
aaaaaattcaaaacaaaatttgtaacagaattcaaaacattcctaaaatattaatgctga
catttggataaggaagacttaatagatactcaagatacaatgtttgttttttttatttta
tttttgtaagtaaaaatttactttcactttctaggaaacactcttagattagttagttgg
agaaatattaaaagggaccttacagcacagccctgctttggttgtgttcacattactgac
tttgtatgagaggttcaccagctgtttgactggcaggagcaggagctttcacttcagtag
tttataattcctggctattctaggatagtttgcacttcaccaagcctcagacaggtcagg
acatttggtaggggaaggttgaaagacaaaagcagcaggccttgggttctcagcctttta
aaaactattattaaatatatatttttaaaatttagtggttagagcttttagtaatgtgcc
tgtattacatgtagagagtattcgtcaaccaagaggagttttaaaatgtcaaaaccggga
aaacctactctaaaccatggcttggttcctgttgatcttaaaagtgcaaaagagcctcta
ccacatcaaactgtgatgaagatatttagcattagcatcattgcccaaggcctccctttt
tgtcgaagacgggtaagtctcacacatttgaaaaatatgaatgtttatttaagtgaagta
ccttggcctcttttagtgaaaagatgcctcaaatttcagtttttatttaaaattaaaact
atcaatgcattgtgtagccagtaaacacttatataagtgatagttttataatatgatata
gagcctagaagatagggttacccagttcacctttttctctcatttatttttcatgaaatt
agaatcctgcagatttatctaaaattagtaaatgtaaaaaggtatgttttgaaatacgtt
gacaaatattagcactgtaaatttgtagtttttgcatgaaaggcaaaaagttgatggtat
tctccttatacatgggaaatacagcatttcattcgttaaaaagtgttccaaatccttagg
cttctttggcctcttaagctgtattatttttttaaattgctattccttacattcaaactt
aataggaacactactttttgctgttatataaaataaaaatagcatgctattgtgtggcag
ttacgatatggtttatatatagacaatcctgaactaacaaacattttgtagaacttgtat
gatctattggatgtttctcaggattacgtactcctctccctggtcaacaagttttaaccc
tgttaacatgagcaattttttgtatttcctgagttaatttaaattgccagtttcaaatta
tttattagagtgatatatttttagatctaacataggtttatgagcctgaaacaaacaaac
aaaccactgcttgggattaaatgagtgtatttgtgtatatcaacactgttagggttctat
ctttttgtgcgtttcttaaagttttattttgtttttaattgacacataattgtattgtac
tttttcttttagctaaaggggaaatattctattaaatagaataatccataacactggaag
aatatttttcctccttaataaagagaatcttctatttgctggaacttaaccttcataaaa
acatattttctagttagcttagttctttttgagtagtttccacttctacgccaaacaagt
ggcagccatgttggaaaacttttacatgaggttaaaatgtaatacagtttaaacatcgaa
aacagaatacaaatatccccagcaccctcttgttacttttgtactttactgttttaagaa
agtgtttgttttattttgtctgctttcaccttgtcattttagtagcttcttctgaatgag
gtcactgctctgatttgagtattttatagaggcctctaatagctttaaagtcactaataa
actaatttattatcttatacccctgtaaaagaaaataccaatacaaataaaattaaaaat
tgagaaaaagaagtgtagggctttttaaaattaaagggttaagatgttaataatcaagtt
aatttttttattgaatatttttaataaacaagtacatatgggagattgcctcattatgtt
acagtatatatttcctgtctttgtcttctaacatggacattaagtttaaaagacatagga
gaagattctgtaagataaattaatagagaactgtaaggctttcctgagggctttctgggg
tcactatttaatttgacttttctaggttactgtgttatgttaaattatagccttgcagtt
gtaatagcagaataaaactaaatattaaaaatgaaattcatattaaaatttttagaagat
atttaaaaaacaaaatgaacacattcaattttagcctgccggccaataatttactattgc
ataactcattgactctgactactagaaatattagagggattgtgaaattatatcctttct
ttccttttgcaaatggttagttgatttacattttctgtgttatttattaactttcccatt
tttctttattagaaattttaaagatatttttaatatgttataattaataatattttgtac
ctgacgtattgtatggcttttacatcgggtatttttactttctttcaagcagattgctat
acaccttgacaacaaagcacacaaagcaaacattcttctgtattttacagaccacttttt
tccttctaagtcatgagctgaatgtaatgtatagttattgcttatgagactatatgcagt
cttgatctattaaaatttgaaagcatggtattttccttccagtcatcatagatactgagt
caagttaatgtttgcacagttaatattttggacagaataaaaaaagtttgaaaatagtgg
aaaaaattttaaggtagcagtaataactataacttcaatttatatctcttcaaatatttt
caggaaattaatttaccttactattaagtgtaatattagtactggtagttaatgtattat
ataaattattctagctgctcatctgagataatttataagcattggatttaatactaacaa
cagtaaataaatagcaacctagctattaatcatttaccttcaaatgaaaatgtttttagg
ccaggcatggtgggtcacgcctgtaatcccaacactttgggggccaaggtaggaggattt
cttgaacccaggagttagaggttgcagtgagctgtgatggcaccactgcactccagcttg
ggtgacagagtgagaccttgcctctgaaaaaaaactaaaattagaagagagaaagaaaaa
aatgtttttagtcttgcagtcctcataatttgtttgtgttttctcagattttcctggtca
taaatatcagctggaatacttactaaaaataagttctgcaggtatttcttagtatcagaa
aaatttgggaatacaatttttaactaaaaaaagccttggttgtcttttaagggaagataa
attttacaggaaaaaattatttaacctataaaataattaggtaaagatatttaaaataac
cagttattttgtaggttatagaaaaataactattttttatttgaaattggaatgcctaat
tatggtaatgttatatttcttaaatcctaagatgcattattttttcacattataagcata
tatgaaataaggatacatcttgatggcactagatagcttacctagcaacatttttttctt
tctagtaataagagaaattatattaagatgcagcttacaactaataattgcatggttttt
tgttttgctttttttgggacagtgtctcactctgtctcccaggctggagtgtagtggcgc
catcttggcttactgcagccttcatctcctgggtgcaagagatcctcccacctcagcctc
ccaagtagctgggactacaggtgcatgccaccgcatctggctaatttttgtatttttgta
gagatggggtttcaccatgttgcccaggctggtcttgaactcctggactcaaatgtttta
tctgctcgcctcagcctccaaaagtgctgggactacaggcatgaaccaccatgcccggca
taataagtgtatgttgaaagaaaatggacttttaatgcataaatttcatctcttgactca
ttttgtttttgtttggagcacttttaagcatttagaatattctcttaaaattattcttca
gtgttactattttagtagaaatttccaaatgacatgcaattgttggtttatttgggactt
aaatatatctggtttgactgctggtttatagacaaatattgtatttagagtgcctcctat
attcttgagtttgtgtagcataggcatcagcaacacaggagagaatggagtagagctttg
gtggaaaggatggagcacagataagggacaaggatttatgcatctgagacccaggaactc
aggaaatagtgaaatatacaactgacgtttccagtagttgaaaataatttcatagtgatt
atattctatatcaaattctattccaaatactgtacttaaaattatttttctttaaaataa
aaatagaaataaaagtagaatatttgtgtgtcatttgaaaaggtgttactgtcacatctt
atgtatcgtcatcttaaatcaggagtccacatgctttttctgcaaagggccagtaaatat
tttaggctttgtggaccgtgtagttggcctctgttgcactcagctctctctgcctttcta
tcatgaaagcagccacagacagtacttaaacaaataggcatgactgtgttttaataaaat
gttatttataaaaacagtgaaaaacaggtttttactgttacaacagtactgtagtaaatt
ttcttttgcatatagctttttgcttgtgtaagtttcattttatggtgagtttttagaatg
gggttctggagctttaagccattactttcctttataagactcattttctctagttccttt
gtgtttgtgggaggaagaattttatttttgcgtatctaatcactctgatttttttttttt
ttttttgagacagactcttcctctgtcacctaggctggtgcgatcttagcttactgcaac
cttctccttccaggttcaagcgattatcctgccgcagcctcctgagtagctgggactata
ggtgcccaccaccatgcccagctaacttttttttttttttttgatatttttagtagagac
agggctccaccatgttggccaggctggtctcgaactcctgaccgcagttgatccacctgc
ctcggtctcccagagggctgggattacaggcagagccaccgtgcctggcctaatcactag
aattttaacatactgctttttattatttatcttctaaatgtataataatggtattgatga
tttctatcattttaaaagagccaactatgtattctaggtactcctcagctactttttata
ttttaatccatttaatccttaaatagtttaaatgttaatgaaacaatgtaagttttatct
ccattcagtagatgagaaaactgaggctcagacaagtgaataactctcccagttttccag
gtagttcggaacgctggtatttaatctcaggactctctaatgctgcaggtactggctgtt
cttttactatgctttgtaacatatacattttttttcttgtgaacttaacacgtttcaact
attagtattttgtagggttagtctgggggcctattgctgttgacattgtttctatgagac
aatacaaagtttcaaacagttgacttacaaataatttttgtagcaccgtccctttataaa
tttaaaattttttgtgtaatcccttttaagttttttttttcctttttagttatagagtta
tctcacagattatgtaacaattttagtcatgaaaaaactggaaatattcagaccgtatgc
atgtttgtcctgtgtctttttgtaaactgagggatgatacttcagggtctggggtcaggc
aactgagaacagggtgcctaacaggtgactatcctcaaaagacaatgcagtaccattctc
atcagttaatcaattagtacttttgacaattatgaccaaaaaaaaacacagcacctctca
taggacctcttagcaatactgtgttaggttcaggtttttcttcattgttcagttattcat
gataatagacaagcctatattcaaaattttaatcttctcattctcataacaaattagtcc
ataacctaatttgttgtttttgtatgtataaacttcagttagttcaaggggtgtataatt
ttaatctattatactcctgtagtagactgttgggtttgttagaacttttcagcaaggtct
gctaggcttattaaacagtcgtattgcttttgtttcagaaaataacatctctaattattt
attctacaataaaattttcattaatatgaagattttatacatacccaaaacaccgaccat
cataagctctgttggtaaagcaaatttgggaattattaccctcccaaatttagtaattta
atatcctccaaagtctgagttcaatggctatataatgaatttttatttccctccaatatt
taatatcttttacttctctttataaaactattagacatgttaaatatgtaatgtagaaaa
ggcacttgtctttacagtgtcctaaattattagtaatctcacgggtcagaaataaccaca
ataaggattttaagcatataattttttaaacaaaactgccactttaattcatttttaata
tactgctagtattttcccatggctgtaggtattcaaaagtatataatctgccatataaat
atatcgtaatttctcactattgttgatttagattgtgacctgctttgaacatcgttatgc
ataaatttaaccacatcactgtacacagatttctacaagtagttttattgggtcaaggag
tgtgaacttctttagattcttgataaatattattaaattttttcagaaaaaattgtatca
gttcaaaaacagcatggggtttctaatgttacaagacattatattttaaagcacatttat
tcttatggcaaagctttgtggactttacttagctgatactgtatcattgtacttccatgt
ttattattctaggttttctgtgaactgaattttcattttgagaaaataacgatatatgtt
tttacttaaggcctcataaggaaaatatatcaattgattaaaaaattatttgtcgctcat
agtgattttattttttattcttataggagataatatttgccatagacttgtcacctatat
tattttcaactgtaattttcttctattcttttcatcgttttaaaactgtgatctgtgttt
ttatctaaatagatgacccacatgtatgatttgataatttattattgattcaacatattt
tttatatggagagtcttatggttgtctcattttttgtcacattagattatacttttacat
ttctgaattttagcatggtatctttacagtataagtgacaacctctcccaaagtatatag
ctattaataatctctactgtaagagtacttctcatgctagcaggagcagctgccagaaat
acttgcctgtttcctatagcactgttacttagcattacatttctgtcatagctttgtcat
ttagccccaatcctggtttgccttaaaaaaaaaaattctagtctagctatgattaattct
catttaaagtagttgtagcaagtctcctcttctaaaatgtcttgatgactaaaagggatt
tcacgaaactcaacatcactttcatagaactcttagccaagtaaaaatatattgatacct
ccttaacatacggaaatataaaacgttttaaaagccagcatcatccccccccctcctgta
actagtctatttggtatccactggttacatttgtgcctgtttttctacatcctttccatg
acctcatggtccagcttacctctctcttgaaacctcccttctggcactggtttcatccac
tgttgtcctcaattattgctgttatctttggtccttaggcatactatatgggctgctcaa
tgatgttatatgacaactttgtatagatatctccttggttaggttataaactcttacaat
tcagaggttatttttaatacctttttatgcttccagagatatgcaggagaaagttagtat
aaatatttctatggctgattcattgaagtgtctgttaaacttggaaaaatccttaaggaa
tttttgtgctgtaagaatgtggcttgtttttgtttactaaatgtttttaagtgactgtgc
agctgtgattttgaatatttcattttctaaatttaacgaaactcagatacagtttgacac
ttaattttaaaagtaattactatgtactaattttgaaaatgaaaaagaaacacacatggt
atagtcatttaggtggtacttttgggagtctagagtagctcctaaatatcagaaaatgct
gaaatagagaaaaaaaatacagtactttgaagagaagaaagaatagggtggtggtatgag
caaatttagagagggaatttaatagggtacctgttttgtggtggggtttttttggtttgt
tttgtttttgtttgtttgtttttgagtgaggaaactaaaatgtggctttctaagcttctc
ttacttgcatatatcttccattggtacttggaaaatataaacatttccagatcctttggt
gttttttatggtcactacttactttatcaataaaggcattaattactgtcagttatcaca
tatcagaccattctattttagatcctttggtctttttctctttaagaatgtttcacacat
catgcatcaacatgagtaagcaatgtaattgtcggttttctttgcattctctgtgctgtg
caagccaccggctataaataagatgatgatcccccatgcccatgcacaaatgaaaagtca
cagtgataaaactacaaacaaaccattatagtacaaaataataactgcttaaatggacct
acgtacaaagtactgtcagagtatgaggaggcactcagagctcaatatgagtggttcagg
aagggcttcacagtaaccctgaattaaaacatgaagatgtttgctaagtaggtgatcaga
ggagtgatagagggaacttgtatcattgcttgggatcgtgtcttgtcttatgttttttca
tattaagtatactgctgcaaattacatcttgccgtcaatttattaatgttgaaaaaatac
gtattgagcacctagagtagagaactttcctagaaactgtagatataacagtgaacttga
tagacaaaggccatgccctcatggagcttagattgtagtttggggagacagaaaataaat
actactgttaatattttgatgacagtaaaatagggtaatagaataaggtatgatttggga
tgtagaagaggcactactctagttactttctatacataaactcatttaatccttaaaatc
accttgtgaagtacgttgtattgtattttacctgttttacacgtgaggaatctcaggcat
aggaaggttcttgccccttagctagtaagtaatgtagctaggacactaactcaagcagtc
ttattccagcatcacagccactgcaggaaacggatctggagtaggcatttgttgaacaga
cccttagtgtccaaatgttttaagggttaacttttgccatataatcttggaattttaaat
gtgtaattctcacctttcccaacaatttatgatggtctcaatatctaattactgataata
ctgccttctgtatgtttttgtcatcagatgatgttttagtattgggttgtgccctagttg
tcagataggagtacaaagcgacttagcacatgatttataaatctgtaactacactatgga
aacaacttgtatgattttgtcttttatgtaacaaatttttaattaggtaacaatttgtat
aattttgacatcaaagggcaaggctatttaaatttgtaaatataaactaagttaggtttt
acagaaactaacttaatgttgaggcttgtaatatagaagacagatgaaaaatatgctaaa
aataaacttggaactaagctcctggcaaggtaaacaagtgtgaaataagtccacagtccc
ttgtctgcaaagcagaaatccatcatgctctgaaaactgaaaatttaaaaataaaggtag
caaaacctaacccaaactaacataaaacagtcatctttattatttatctgaatattcata
tattttgctggagaaacactaatgtgtgatatactggaagtgctctgtaataaatagtat
atgcactctgttacctttctacaattcaaaatattctgaatttaggaacacagctgtctc
caagagcatggaactaaatccagagaccttagagttttacaactgtcacccactcataaa
aaactgaacacactttgaaatgtcagagtagtctaagctttatcttaagtctctgagtac
atgcatattaaatactttaaaacatacatatatagatgagtacctaaagtttattaacct
aattataggctattataaccatataatacaatgatagtttggagcttttatattactttt
cttcagtgattaaaatacagctcatgctaatagacttattctttaaatgagtgtagagaa
cctcaaccacattttttttaattgtcccagtatttccatgggaaattaacaccacaaaat
taattttaatctgctgatatatagtatgcatactctctgtgtgtgttgctactgttacaa
agatgaataagacagcacacctttcccttaaggaatccacaaatgccttcatgtctctga
gagacttgtctcatcatccacatttcatggtcacatatcaaaattagatgaacatggctt
aattaatttattcaacaaataagcatctgctaatagccaggcccagttctaggcacttgg
gatacattagtgaacaaaacaggaaaaaaagtcctttcaacattttagcggtcctgagaa
gtcctccaaggcaagaggcagtttgtgccaacatacagtgtgtcatcagactcctttatt
aaattatacacttttcatttctaaatcatttggtgataatttgtattatagttttttcat
atgtatacataactacatacatgtacatacaatttacctgatttgatagaattaagttag
tggccttattgaaatgtataatctgccggttttcaaatactgttagatagtatctattac
aagcaaaatattattttgatgctctgaaatgataaataggattccatttaactccctttt
taaatatttttgtgaaacaactttcttttggtgtttttaataattctatcaggcacagct
tagtgatttctttctgaattattcctggatcagaattagacttcttgtctttttccatgc
cactatggaaggttgcagaaactagcatgtatctcacatataaattagttatataatggt
agataaaatttatggtagatatatttgtgttatattttggtataaataacagtagatata
atttaagatttttaaagatgatattaattttactatagaaataatagtggaaaggcccag
catttttggtaaagttgatgacattctaaatgttagcagtttcaattcagttatttcctt
tcacaagtacctgctgatgcagcacgtctctttatttgtatatatagtgttttactccta
aaatatgaagggtaaaattattaaagtagggtttttttaagtcaatatcttagattaata
aagttttcttttgtgactttgatagtgtcttgtgtttatatatttataaacttcattgca
atttaggaatgggggaagcagtgagtctttctcaaatattaatactgtttttgtaattct
gtttttctatactgaatatttttcagagctatcgaaccataaaatagcaggcttttcttc
ttaggagagccatcatttctcaaacgtacatcttactacattggcattatttgcatattt
gtttttctttgagagagagaatagttgaaagtggccattgaaagtagcacctccaagtgt
agataatgctaaaaacattgcagtgatccactatacatgtaatattttgatataattaac
ttgaaacattaattttatagtggaattccatgtttgcattcattaggaatttactcacaa
ctaaatcaagacttttaatttgaactttcttcctcaagtaaaggtagcactccccacccc
atacataaaagagacagaaaaggttaaatatttatcccaaaagcacatggctaattagcc
atttgtttatgattattttgtggtatcctcatataatgatgggaaaatctgttttgagga
tttgagaaaatttatggtaatgttgaatgggattttcttaaatttcttctcctttgctag
tcttgaacatgaaaattgaaacttatgagctatttaattacatttttatgtgtgggtgat
ttcatatagttcacttgggaacaatttcagcttaaacagcttttcaataatccatagaaa
tctctgcgttatcactttagattttaaaatgtcactgttgctactaatatgaacatgtag
gggcagtatagtactgtttttgtaaatcttgtctgaatttgtgagttttaaaaatctgtg
ttactgttagaagtttgttcttctctaagataacataactatatcttgcttttttaaagg
atggttggtactgcttcattaagttggaactattctttcagttcatgttcttacatgtgt
tatacaaaatctcttatattatgaaagctcttggccctgatttatatttgaagtcaagga
atatgtttagttaaaaggaaaaatgcatttttattgtctgacaataaaaatgagaataaa
tggattcataaaaaccggaaaataccaagatcagtgcagttttgcccttactctgtgtca
tcctttcaaaatgaatgtttatgataatttttacagtcttaatttttcaatatgacaagt
gaacaaaactgagattaccgaacagtcttatttttaagattctttaattggtacatcttt
ttttcgtgtcaaaatggttaaggttattacaaactttattacaaaatatagtaacttttc
ccccgacttaatatttaaaagaagacaactgagcagttatctcttccatctatgtattca
ctttattcaacacctcctatgagccacggactgctttaggcatttagagtacaacagtga
acaaaacatcccagccatcaccctcgtggagttcatatcagaaaagcagagtgcctaaga
gcgcggattatagggtcagttggacctgggtttaagttctttgctgtaccacttactaag
ccttatctgtgaagagctaatagtagtaccttttacagagggttgttgtaaggataaaat
gaaataacccatggaaagcatggttatgtacaagagttctgtaactattatgaagccctc
ataagacatttttagatgtgttggtaaaatttataattaattctcctgatgtaatagtac
ttatgatagagattttgagtatttttttcatttttagtttcttcaagtggtaaaagaaga
tgcacagcataaattttgaataatttttcttaaacagtttcctgagtgacttttaatctt
ctctgagtggaaagataccatgcataaaaagttaatgtgatttggttctacttacattat
gtagattcattcagtgcgactatcatatatgtaaaaattaaccccattatgtctccctac
atgaagaaaagcatatgtgacatcttggtagtttgtttggagattctcagatgttcagaa
taaatgtgttggtttcaaatgtcattaaattggaatttttaaaatcgctatataagaatt
aaaaatacatgttattgatttacatattatgttagctgctaaaatgtctagtatatagtt
tccattataattttaagttctgacaccattttcctactgaagcccccaaaatttggtaat
taagaaatgtaacatactttttaacaaatttgaaaactgtcttgtcaaaatggtacctga
tatatcattactacataagaattgagacttaaaacatttaaagaactgattcacaaattt
taacaaaagtacttagaaaattagacctttctttagtaccctgtgcttctcttggttctt
gttatttctgtgttatacagaaattccatatttaacacacacacatgcatgcatccgcac
acacgggcatcccacacacatagaagtaaaattctaaccaccccctacctctatttaata
gcttatcagctatgttcatgctacaactgcttcattgaagggaagtcctattctgctgtt
ttcatattagctttgttataatgcagtatttttcagctgctcatgctcattaccatacag
ctggtatgtagagaagagaaggaaacaggcagaagttgtactgtctacaagaataagaag
ctctcttgcagatggggtgctggcagatggggcatgtgtttgaacaggttttactaataa
cagagtgcgttaaaaatcattctgagaaagtcctaaggtcagtgccatggattcagagct
tgagaggttgttcagctttttcgtagacctttttagaatattaatcgactcttacaaaaa
aaaagtaataaatggcaaaccatacctcctccaaaaaaaacttaccagtttttttcttct
ttatattagtatgttttttctttagtataattttaaaagcattaatactgtcacctagta
tcatcaacttgtgttgtcgtagtcttaaatatatttacttaaaatgtctacatctatata
caaaaatgtagatgctttgtgaaaatggtaataacattcttcagtttctgtatattagaa
tatttattagaaagtgaaaactaaagatttagatatcagtgtgtatagtgttgactacgt
tgaagattaagtcgaggaatgttttatctgcatacaaagttagaagtgaatggtatgccc
agaaatgtatctattgttaaaaatatttcttgcatattttgcatattcacaaaaccaaag
tgaatgaagtataattaaaataaccatcatttttattagagtattttagtgggatcttag
tctttttggcccttttttattaactagtatttcttgatatatcctctctcaatgggagat
gctgatgaaaagcaactgaagcaaaataaagctaggcaattttgtgtaacatatgtagta
ttcaatttaattttaaaggaaatagaacttgttcttttcatagagattaaaatatgtgaa
tatatataatatatatgaaatgtatacaagtatagctagtttttttttatactttaagtt
ctagggtacatgtgcacaacgtgcaggtttgttacatatgtatacatgtgccatgttggt
ttgctgtacccattaacttgtcatttacattagatatttctcctaatgctatccttgccc
catccctcaaccccacgacaggccccagtgtgtgatgttcccagccctgtgtccaaatgt
tctcattgttcaattcccacctatgagtgagaacatgcggtgtttggttttctgtccttg
tgatagtttgctcagagtgatggtttccatcttcatccatgtccctacaaaggacatgaa
ctcatccttttttatggctgcatagtattccatgatgtatatttgccacattttcttaat
ccagtctatcattgatggacatttgggttggttgcaagtctttgctattgtgaatagtgc
cacaataaacatacgtgtgcatgtgtctttattgtagcatgatttataatcctttgggta
tatacccagtaatgggattgctgggtcaaatagtatctagatccttgaagaatcgccaca
ctgtcttccacaatggttgaactagtttatagtcccaccaacagtgtaaaagagttccta
tttcttctccacatcgtctccagcacctgttgtttcctgactttttaatgatcgccattc
taactggtgtgagatggtatctcattgtggttttgatttacatttagtttttaaagctag
tataagttacaacacttttggcaactcagaaactcatgcttcagatttcttgtcagtaaa
atggaggtacaaaatactgtctgtgaagttaataactatgaataaataaaatgatttaaa
tgagacctgtgagaagtggtgtactttaacaagcattgctaaaactgtgtattcctttta
atagtgaaagtttagattctgttgggcattttaaaggtacagaagtcctaaactgtgaag
ttgcatttttattgctcatcaaacttattttcttccaatttaatttgtatttggactgta
tgactgataaatatgtagaaatgcttttatgacttttccctgagggggaaatatattaag
tattataaacaatagacagtataattataaaatgataaacctgtaaaatgtatttttcac
ctacatcatcatttcttatttagaaaattatgagtttttaaaattgtgttcaaaattaat
ggttttctgttgttatttctgtagctctaccattgttgattagggtatgtcttgtgtggc
taattatctcatataaatattttttattggtaccctactatatgcaggaactatgtgagg
agctgagatacaacaagaatcaaaacagaataaaaatactgccctcatggaacttacagt
ctattggagaagacagacaaatatttaatgtgctcacactgtaacaaggtgttggcttca
agcactgggaaatcagagatagcctgtatacagataattcacatattttcatctccaacc
cagacctttcttctgggccagaaccagttggagcatgagatttggatacccagttatctc
caagatatctcaagatcctcctcacccctggcccccagaaaacaaaaaacaaccaactaa
cccagtccttgttatccaattgcacaagtgagaaaacaggtgttatctttgataccttcc
tcttttttacttgccatgcatgacccatctgttactatcctatcagtttgatgtcctaaa
tgtctcttaactgcccactcctatgcatctctgtttctacctagctaatcgaagccatca
ttatctcttgcctggtctagtggaataacttcctcacacagtctctccacatctactctc
aatcactgccattctattttcagcattacactttgaatgatttttttcaagaaataacat
ctgattatatctttttcttaactactatgcagcattagcttttcattactcttatggtaa
aacccaaaatcctttgaggtcttgggtaatgtatcccttgtctacttcttcagcctcatc
ttacacctgccatcctacctctgttcagtgtcctctagctataggtggttatccctccct
gctccgaacctagctcagtcattcattcagcgagaagatgccttctgtgaacccacagac
taggtggaaacctcaccattcttaacactcttcgaacactgcagcatttaccacagttgc
agttttgctttgttttgtctcattatcttgcccactatgctgtgaactccagccatgaaa
cagacacaagacttagtttagcttatgttaggcaactaagtaactattatattgaatgaa
tgggagtatctgtggcagactgaaataaccatgtaaatctgcatgtgttgtaattatgta
tattaggtagaatataattatggagatacttgagacaaagtgtagggtagaatcacagag
atacatatgtttcaatacagatgttcactgttaggaatgataaccatacatggatcaaat
ggacaattaatgaagatggattggctctttaaaaaaggccttatgtaatgaagttaagaa
acagggcccaaatgatagccacatattgaaaagcatgtgctctatgtattcagatggaaa
atcctgtgtatttgttactacttggttagtaagtcaagtatttttaggctgtgtactaaa
aaaattttcaatgaagaagtcatagtcccttattaagtgtatttgggtttgagtaatcaa
caaagaacatttgaagcagtttctttcataaatattatgattcataacttttgccagttt
tccatagggttcaaatcattttggtaaggaggaaaaaacctagtagcttcgttgaaagga
cactgcaatggagatcctttgatgtttgtgttttcagctctcttttatttcatattttag
ggaaataaagcaggacatgaaggaaggagagtaagacatagaagtcctgagagggtttgg
gaaaaggaagcaaaacccatttgttgttgtgtttcttccagtatgcccactgatgactgc
ctggcctcaatataatgtcacagattataagagcagttcttaaccaagcggagccactgg
cagtagtgtattaacttgttgtcaagctgctcctggtgaattcccagaaataaagtgtat
cacagtacacaattagctgcatagtccgtctcttctttctgcaagtctgcagtggctcta
gtgtggatttcatattcacatcacacatttgcatactgaaataactaagaaaacatctta
gagatatttaaactgccaatatttcttgctttgtatatagagaaccctatttttattttt
ttgtaaactgaagaccaggacttccttaggttcaaatcccatctaccttaagtgaccttg
gggtcgcttaaaactctctgtgcctctttcctcatctgtaaaatagagataataatagta
ctttctgcagaaagttgttaaaccgtttcattgaattaatataagaaaaacacctaggaa
gagtgcctgacatatagtgagtttccttccctgtatacattgtattcttaagaaaacatc
tttttgaaaaggagttgcttattcaaactggaataaagtttgcagcatctttgatagtgg
gcactgaaagagcattcaggtcagtttaaaatctagtgtttatttttcttgtatacattt
ggtttaaaatattgccatgtgcatttcttgaaaatattatctcttcctctttctagttgt
atatattgtcatacaaattaagcagtttcacttattagtcaaatggatttgtcatttttt
attgacttgcctgtccttccctcctgagttgggagtgcttccaggatagatattacatct
tgctcaattttgcatcttttttgcttagtgcttggcttttagtaaccctggacaaatatt
tctaaaatgtggtagatctagatctaaaaatataacatttgaatcactgcttaatttttt
ctccctcttgttttattgtttagaaaatggagaatcattatatgactcaggattcatttg
gatgagactgataatattataatggaatttggagttagaggctcacatgaattgctctca
gtcactagctttttctgtgctacctataaatgataaacaaatattgtgaatctcacaaag
ttaaagtctttgaaataaatggattggtagggggtaaattacatcttagtctaacaaaat
ctgtttattgcaatggtttaataaaagtcaggtgactaaaaatatcttggaaaataccac
ttaattttaatctttgctgtttatgttgtgatcatgatgattaacatatcttcatttatc
ttctcttgaaaactattccaagttcttgttgtttttttttttttttttttttgagagaga
gagtttcactctgttgcccaggctgcagtacagtggtgcaatcttggctcactgcatcct
ctgcctcccgggtttgagcaattctctgcctcagcctcccaagtagctgagattacaggt
gcccaccaccatgcctggctaatttgttttatattttcagtggagacggggtttcaccat
cttggccaggtgatggcctggtgaacttgatcttgaactcgtgaccttatgatccacctg
cctcagcctcccaaagtgctgggattacaggcatgagccactgcgcccggccctaagttc
ttttttaatatgctaagttgtctgttgttggaatgactttaaaggaaaggtttaaaatga
agaaaggaatatgtgtgtgtcttctttatcatctcatgaactaaaaaatactggaatttt
aagattacactaaagttcagcatccttttgctagctttgaattgatattcttttgggtag
atttagactattcctcataggattttttcatattgtataattatgaacatacattgactc
ataaacttagattttaagaaatctaagatttagctttaaaaagtggtctcaatttaccat
gtagcagttcgtaacagaccacagctatatgcacttgttcatatcaggagcagttcttaa
ttttagttataattcatatttgtttaatagtcatcattccatcattacataccatgcctt
catgcaactggtgggaaacttatgatattcattccacataaaggagtttttagaatcatg
tattaaatgtgataagctttcttttttcttttcttttttctgttctgttctgttcttttt
tttttttttttttttgagatggagtctagctctgtctcccaggctggagtgcagtgttgc
aatctcggctcactgcaccctccacctcccgagttcaagcaattctcctgccttagcctc
ccaagtagctgggattacaggtgcacaccaccatacccggctaatttttgtatttttagt
agagtcggggtttcactcttggccaggctggtctccacctcaagtgatccaccagccttg
gtctcccaaaatgctgggattacaggtgtgagccaccatgcctgtcaaaagttagttttc
aaacatttggaggtctcaagtgtttataggtaaatattttttaaatcatgtgctaaaata
aattttaagaaagtgaatctgtaaataaaatatttctatgagaatgttgagaagatagca
aagtgcttctaactatcagtcttacagttcactgcacataaaagacaaatatacattcag
ataccttttcaagttaagttgtcttgaaaatatttattgaccttcagagtcttttggaat
ctcctaaacaggtagggaatcatgtaagtatttgctagtcatagaattcttgattttttt
ttaaagcaaataatgtaatttgttaaagtatatatatgtatataggaagtactttaaaat
ttaaattaatgttttaaaaatgaatagaatcctctttttaaaatgtagtgggggcctgat
tactatgttcttccctactcacttgacagctatgtcaaacacttgaatacttaaagaaca
agcaagctacataaaaacaaagaattgattaaagatcaccatgaggcacactttctagag
caagaatccagtgttatttttgtttctcactggcctagcaccaaaacaaaataggtactc
aataaatatttttgaattagtgaaatcagtttgaattcacagactttgattcataactag
agttttaaaagattggttcaagttatgccttgtcattttgaattcttctggaaaattagt
aagatgcttgatgagaagagatgcctatattttaaaaagtgcagtaagtatgaatctgga
ctttctgtatctgccagccaaatgttggctggcattttactatattttatatatatatat
atatttaatatattttaatatatttttctaggcttttctctatgcatacacagtgcgcac
acacataaaatatatgcttgttttttcaaaaggaccgtaccttatattgtgttatcgaat
ctcacataccttctagttttacagtcacccttccatagtcacctagctctgtacctgatg
aacggtttctgagacttcaaaatgctctatagtgaagtctgttctttagaaaagaggctt
tgagtctgggggccagacagtagaggtgtggggagaaagggctggggtagaacaagatga
aaattttcttagtgagctatacctttttgaataggacaggttctttcagtcacatgttgg
caagaatcggtgaattgtgtactcacctgttgccccgagtaacttttcggattgtttttt
cattaactttttctgggaatatacaatttcaatgaatgttttatacaccatttccctgcg
ctgtgtattcaggagtacaacttgcttgtatgtatgatttctagtgtgaatgcctgttca
aacagaaagtccccttgtttgtcagtgtttgtaactaaaataaaaagtcaacagtcttca
cacccacacagtaaagttctcctagtacagaaagacacataatgccctaagatggaatga
tgagtcatgacaggaggtattttgaagaaatttaacaatatgttaaataacattttaact
ctagtattaaatgtttcagttaggagaaataattgttatcacatgggttatcccctaatg
aaccttgtaagaccagtgattagaagtctaatagatgctgttaatcctgcaagttaataa
tagtcgtatttccatttacatctcaggtccacaggtggtaatttccttcttccctctcaa
cacttatcaaacttctgttttttaaacacagaagcaagaccatgtcatttatgaactccc
agactattaaaataacttctccattttctgactcactccacccaatttggcaattgatat
atagtccagtattgctttctatttgtttcttgtctttgtgggttttttttgttttttgag
actgagttttgctctttttgcccaggctggagtgcaacggcacgatctcagctcactgca
acctctgccttccgggttcaagtgattctcttgcctcacctgtcttcgtggtttttgttg
ttgttttgttttgttttgttttgtttttggtagtttttgtttgtttgtttttgagacaga
gtcttgccttatcacccaggctggagtagagtgctgtgatcatagctaactgcaacctca
aacgatcctcctgccccagcctctcaagtagctagggctacaggtgcacactaccatgcc
caggtaattttttttcattatttatagaaatggggtcttgctatattgcccaggctggtc
ttgaactcgtagcctcaaatgatcctcctgcctcggcctcccaaagctctgggattatgg
gtgtgagccaccacacccagcctcatttcttatcttgtcacctggattttaagtatcttg
tgggaggaaggtaattgcaatgtttttgtttgcccttggtagcatttcgcagatattggg
acataggaggtgacttatttctcaattgattgtttttttaacctaaaattttctcagttt
ctgtcacctttcccccatcaccaggcctttgcctcagatttcccagatgtagtagaagca
tagttaatatggacctaaaataacgattcaataaactgaataatcaagatttacttgtaa
tttacatacatactttttgcaaaatataacacttttttcttctttaacaatgtctcgtgt
tttagcttaacttgagctaccacatatctaatggaaatcagtaagaattattgatcttgg
gaccagagaccaaggttctaaatatagctagctatttaagcatgggcaagacacttaatt
ctgtacctacttttcccacctgttaaatacagctaccatttctagggtatctgttatgtg
ctaggcattttctgtttcctcacagtgactctgtagtttaggtggtaaggtaggtgtata
tcattactgttttataaaaagagaaaacaaactcgtaaaggtcaagtaaccacctaacat
tacactctggtaatggcaaaacttgcgcttaaatccaggtttttctggttccattgcttg
tgttcattccacaaacaaagaggaattagatgacatccaaaatcactctatggcttcctg
ctgttaatattttttgctgatttagtagctataccttttttttttttttagtctttagct
ctcttctcaaaacttttttatcatgactaatcactaatttaggaaattaatcccagaatt
ttaaacagttcttgaaatgaatacaagattaatatatcacacctatgggaaattcttctc
aagtcaggcaaatatatttgaaaatcacaaacagcagcaataaatatggatttgtgtaaa
gaaagcagaagagtgaatagggtttaggccttatattgagggaaatttgatttcattctt
tctccatgttaaattctttcctgggtcatatttgtggtttcatttttcttgaagtcaaac
ttacattttatactttttttccatgatatctgtcttactttactgaaggtattactataa
gaaattcacaataaaattttgagaggaaacttatgaattccaaatgcttattggtagttt
attttggattttaagcatgaaagattcattgctttctggataaatcagtaaactccaggg
tgacttgctaactttgttgtcaaattattgcactgatgccggctgtctgtgaaaaagtag
aggatgttgacttagtattagtctgacagctctgtaacaatataaattttagaggagcga
ataaaacccccatattattttattctgtggctggcataaatagaactcttaacttgcact
attgggtatgaattatgtatgtgtgtatatttatatatttgtagataaatacatttgtgc
atgtgtccatcagatgactaatttggcccaatctgaaggaatgaaaaaacttacactgtt
ggaactaatactgcagttgattagtgtgtcacactaatcagaggctgtgtctttgtaagg
agagttttaaatagcttaaacttcaagaattaaaacttctggccaggcacggtggcttac
ccctgtaatcccagcaccttgggaggctgaggcgggcggattacttgaggccaggagttc
gagactagcctggccaacatggtgaaaccccatctctactaaaaatagaaaaattagtgg
ggtgtggtggcacacgactgtaatcccagctacttgggaggctgagtcatgagaacctct
tgaacccaggaagtggaggttgcaatgagccgagattgcgccactgcactctagcatggg
caacagagtatgactatgtctttaaaaaaaaaaaagaaaaaactactacttctgtaatta
tattaaaatttatttaaaagcagtcagctataacattttgatttgcagcttgatttacaa
aggtttggattatatatattataaacatccccttccccccaaaaaataagtttaagtaaa
gtaagattatctattacagtgtttagtgtttgggatttttttctaaggcactagtgtcac
agtatttgtttacttagatggtagttttgaactaaagctggctcatcttgttttatttta
catgttctcaaaaattttttagaactaatttcagtatgagttctaacagtcagatgtttg
tgtatattacgttacactatgctcttgtgttaattgactgtatttccaatggagaaagaa
caaagtttcaattgatgtatataatattctgtgcccaacacaaagtaggtgctaaacaaa
tgcttactgaatgaataaatatggaaaattgcttattggccaatactgccacctatttgt
gttagtgaaaatgtaaatgctgggcatatatactattccagttgatatctgtaatagaac
aggtttattagtttgccagttttaataagcaaaccttttaataagcactcacatcccaga
aagttaatgacagtgagttagtgaaatttcaaaaagtatttcagtctcaacatgcttggc
attcctttcattgatgttgaaattttgaactgtctgtaattttgaactgtctgtaatcta
aattctgtgctcagggcaagacagctaactgtggtggtttcctagtcctttctagagtac
aaagtgttgtcatgataataataaaatcactgacataggaatataaacacattctcctac
tattataaaatggaagtgtatttttaaatgttagcaaggtaaagaaaagaactaatagta
ttcatagtattttccactaattataaactttttagttgagttaaactttttagtactgat
tgtaatatatgtttaagctttagaaaaataatgctgtggtttctgatatttgcttgtatg
gccaattcaaaaatattaattatttgttttgtatcattttctggatgccctagaaataaa
tttgagtaagatgtgaatctttagggaggagataggggagaaagatgtataaaaataatg
acaatgaactatgataactgcagtaatagaactatatataagacggaaataatgaactct
tgagagtcaaggtgaggtaaagtactctattaactacatcctttatgttgacattatatg
taaagataagtatttctctgaagatacatgattcgcttaagaccatttgtgtatattttt
ctcactgtatatacactattttttctttctgctcacaaccacaacttgcccttctaaaaa
cttaagtcgtcccaattatttgttcagagctctggtaggagctcacgggttaatgattct
atggcatcattccccagaggcaaacatatcatcatatatttgtgttgtttttttttcctc
gttacttctaacttctatgctagcacactagttccacttctacacctagcacagtggagt
tgtgatgagatgagcattgggtctgagttagcagatggattttattccaggaccttattg
ttggtcattccctttgctaatggaatttcaataatgcttctaggttgtcagtcaacacaa
gaattaattttttatagaatgggagaaatgagtgcctagctgaaattccatgaaaattat
tgtaatgaaaatatatttaatatagtgaaatagtaattctaaaaatatagatgacaaaca
ttttcttagctgtttggttgactaacttagctatttggtttcttagctatttggttgacc
ttctttcctgtgaacttactacttaatattttagtgtaaggcataaaatactttaaaaaa
acttatttaacatagataatagactgatctaaagtttaaaatgtttcttgtcacagagat
aagatagataaatacagacacaaacccataaaaatttttaaatattatattaccaaaaac
ttaaatctgaataaagtttcaaactatattttagccgagtggtataaatagaaatgcaag
tgattttatttgctttgttcatattaagattatgatgacagtaatataatagatcacagt
tttcattgatagtacgtcagagaatgttgaaaatcatttttattctgttaatttgaaatt
cagttagaagcttagttgactagttttgcttgacagattcttcacagttcaaattaacat
cataagatattagggagaattgtttaaatacttgggaagacaaggtcttcattagtactt
ataaacattcgcatttctcacttaaaggataaacaataaaaatcattcccaaatgggaac
catggtagatggaggatatgtggtagtagaaagacttctcagtgtagaattttttgtata
tcattttgatgtttgaagcatacaaacctggtaataaaacatggatctacaggacagtac
ccacatacaaacaatatttgtagggaacaggttttcctgtaactcacagccctaagtagg
agccatataaccatgggagcagtcctttgaatatacaaagttaagagatattttctttag
gttttgagacagtatcccctacttcattaaagttggtctcttcttaactgagtagattta
atttgaaaaggttagttcaactagaaataggacatgatttcagttgaagccaaagtatat
actattatagcactagtattacacgtgtcttatggaaaacttgttaccactgacaaattc
tggatgaaatttcaggtgttctcatagcccagaacattgaggaaaatcaccttttttccc
cctttcctaaatcttgagtcctcagaacttctgacaaaagagtttatgggtccattccta
acaattagatttttttgtttttttgcttcccttcgaagtgagattatttagataactaac
ttctgctgagtacatatacatattgttcctgagggtaagaattaatttaagagccaggta
ggagattttttttaaagataagtagacagtagaatataatttctagaagactcaagatgt
acatcttcataaaattttacgttatcttaatttcatttatgttttaaagtaaaagatgtt
gcattgacataatgtcttagttataaaggtaaacatttttatttttaaaattatgatact
actttatgcagtatttagaaccattttactaaagaaagccatttaccaccatagcagaga
gtattaaaatgttagtaagtccaaatggttgttttgttttgtttttctggagaaacttac
ttcgaatagtggtaactataggcacacaatttacttgtaataaagaacactaatggggag
ggtaggagtaaattaactagaaccttaataatgggcttttctagctcaaggaatcttata
attgtccctgaaggtagttgtgaaggaagttgcaagatgaaatatctagatttgtgtagt
agtcattccaggaaaaatgttgccttctggcaaactttggcatctttattatgtaaaatg
cctttttaatgataagcaatatagattgtcaaaataattttggagatagaagtctagtgc
ctcctgtttactccagattaaattagcaaataaaagtaagcctctccagccagcacagtg
tttgctcttttggggataaaatagccttttggccaaactttacacacttaggtagtacta
gtttgttaatcttacaaagatattcttatgattagtaagaattgatgaagtgtatggcaa
tttccctgaaaattactatgttttaaaggattgaaatggaaaagcataggcaatgttttt
attaaatcaataccaaaatacgcagtttgaaatataacttagattacataatttctgcaa
atgctgtcggctccaaaccagtttactttattgtcagtcaaccccagagtatattaattt
ctcttttcgggattgcctgttttgtaccatccaagctacttgaaaaatctgattagtctg
tgccaggactacggtaggatgacagtgaaaatagcatgctttgcctcagttcagggggac
cgtatgaaaatatatcctattttcaaaatgtttctgaaaagacttatttgtggaaatgtt
ccttgctatttaaaactcttcaaaaatgcaggttttacaaaatacaaaaataacctcttg
aaagtttgaaaggggagagggggctgtcgtgtgggggcggtctctgctgcccacatcctc
cctctgttcagtgcgttgtgttcttacgggttccctggagcggatcaccatataattgat
gtgcagtctgcattgctgaatcctggactgcaccattctgtgttcaagggaagatgtaat
ctgatccctctgctgctgagggaggaatctgttcagtcaaggctttgacagggcatagtc
tcctccaataatcttctccgttctctctcattattccctcgagttcttctcagtcaagct
gcatgtatgtatgtgtgtcccgagaagcggtttgatactgagctgcatttgcctttactg
tggagttttgttgccggttctgctccctaatcttccttttctgacgtgcctgagcatgtc
cacattagaatctgtgacatacctacctgaaaaaggtttatattgtcagagactgccaag
cagccggacacacgggggcacagaatcactgaaggggaaaaatacagaaaatatgggttt
ctacggcacattaaaaatgattttttacaaagtaagtaagctgttttcttcctatgctat
gtgattgtgtgtgtatgcctgcctgagaaggaaaatctgtatcatcagagatggctgcag
tatcctctaattttggtgggtggctggatgtctggtattctgttttatatcatgaatgta
agcaaataacggaaagactgtgagctggaaaaagctttaaatttaagataaaatgtatat
tttaatctgagaatttttcactaaaaatacattgagactttagaaatgtggctctgttaa
atgctgacctggaaatactctatccttagttggcttttagtgaaccttttgccttcaatt
tttctataccttgtatacagtttttcttttttcattagttgactctttaaaagtatagaa
tcatgagactatttgggatttatatttaaagagatcatgtttactttctgtacccctcaa
aaattgcagaattaatttttggttctgaagtcagtattttcagtaaacagtacagtattt
aaaagacttctatagtcatacagaaggtgctttcctttataaaatatttttaaaataaaa
tcctttctcagtggtttccttgatacacattttggggcatgtttattcattgaaggattt
ctaagatggaaataaagtttgcttttagtggcaaatggtatttgcaccaatgttttttac
aaggtatcctgggaagttctgaaagccagtattacaaacttaattagaataaatatcaaa
gtttctcctaattatgaagataagaatttgtggaagttatcaaaaaactagattttaaca
tagtctgtactgactgaccttcctgatggattcatttgcatgtatgtttatatatatatg
tgtgtatctataaattatatacacggaaacacgtggctacctatatacttcagcttttat
atttatatttgctttttaataaaattatttggagaaagaaaataatggtttctgagagga
atcactttaaaacaatgtaatgagtatataaacttctaaagggaagaaaccagccagatc
aattccatttctttccagtaggtatggcatgaactaaggtataaattgtaggtgagcttg
ttgtaccctcacctctagcccctaatgtcgtaagatcatcctagtattttagaaaactct
tcgtgatgacaaaaacatggtctttattattcagtgtggctctaaaacattttaagtctc
tttagtatttatattgtatcatgctatgttatgatgttcccatgtaaggcaagaggtagg
aaaataaggctgctgcaaagcaaggatttttacctacttataaacatgaggatagctctc
tatcactttggagtgtttttcctcctataacttgcagtaatacagtgattaattagtgga
cacaaatcttaaaaaggggttgttgaaatggaaaaagtcaaatcttaccagattttattt
tctagttttaaaatatacatatagtcaaatgagaatatttcaatattgatgttgacaaga
aacaattctgaatatatttatagtataacatggcataatgaaacaccttaaaatgtagat
tttggatacaccttatagaagaggcataactgattcttttaaaacctttctgttgttgct
tttcaaattagaatattttgtcctgagtatctgtcaatagagctatattctgtatatata
tcaattgacaactttcccctcagcgttattatactatctccagatttctcttattattga
tttactttttaaaactgtccttattgcccatattaattgctttatgccttatcactattc
aagattatttatactgctctttaccctcttataatggtatgagtgtttaaaattgagttt
gttactttcataattatttctgattttacaattttgaattttatctttattaaaaacatg
tttttaaagaaaaatgattcacgaggaaggcacgttaaatatgattattttctgtgtgga
ttaacttgagatttttccctatgttggttattaaattggaaaacatgaaagatatattat
ctcaaggaatatatatatataatgaatatcagttttcaaatcatcacacactgttcttct
ggataaaatattaggggggaaaattcacctttccattccattcagagtaaaacaaagtag
aacaaatagaagaaactatgaaacatttattttatatatttaattaacattgtaggcata
tgaaaatttaagaatttaaaatattaaccatgctgactcaagatttgatagttagacgat
ttttaaaaaacatatcttgtttgttttaccttatagatgaaaagaaagttggaccatggt
tctgaggtccgctctttttctttgggaaagaaaccatgcaaagtctcagaatatacaagg
taatattaaatataatgattatctcatgattctcagttttttagagttgattctgaaagt
gttttagtttattcttgtagaataataatgttactagtacggttgactgtttaacacatt
ttgagattaccggaaattgagaactatatcattttaaataagaggaatgccaccttagtt
tatgttttgttactaatacattttgaagtaggacacaagcagaaattatcttgtgaacaa
aaatttaaaattacaatttttaaggaattctaaaaagtaaaccataaaatatgaatatga
gcccctgtgtcttgttactataggaacagctgttctagggtagacatttatgtatggaaa
tttgaagatacatttattagcagtgaagtaaaacaattactaactcaatcttatttcatg
aaattaatgtgttagaaattttcttttatgggcttggtaggtcttaagtttttaacacat
acatttcttttctataccagtagaatagtttccataacaatgaaacataattgtaatctt
tttctacttcagtccccattatttttggattttacatcagtataaaacttttcccactct
cacagttattaggctaacttaagcacaatgtctattgcccagccaattaaaagtggcttt
gaatgtcacttggaatgtcgtattttaaaatttaaatgacagaagtatcttagagctttt
ttttttggtctttaaaacagaattttttgtataattgccagtttttaaaataagtgattg
aaagttaacagtagaaaccataagaacttcatggttatgacatttcttcctttctctcaa
aatatttcaatttagttatttttattctataagatgcactaaaattaacattgctcagat
ataatttttttattgaacattgttgcagattgtgcctcttacagagaaatagcttgtttt
tatttcagttgtaaaaaatatttctttcaaatgctgctttatgaaatttgaaaatcatca
taattggaatagtcttacattcaaaaagtgaagagataacacagaaaagaaaaataaagc
tatctcttattcaggagctgcttttcggtgtgatttttcttatgttaactgtcccccttc
ctgttactctttgccttttgacagttttactgtgttttggcacttccaccagagggtgtg
gagggatagagagacaagtgaaactattcacttcattttgcttttcccttctagtcatga
taaagtatttcttggatgagaaatttgcatttaatgttaaactaactgttaatgctattt
tttaaatgtatcagtcttctattaatatggctgataccaggacaaaggtattacattgtt
cacttgtttttaaggcagcttagaataaatgagtgctgaatttttattacagaacacatt
ttagtaaaatttgtctttagttgctcactttttatattgacaaagcaactgaaaaggcag
aagttttatttttagcctgttaatatataattttattaaagcaaaattagaccaaacagc
atatttatttccatgtttttctggagactctcggttaggaaggtatatatttggtatttt
gtgatatttttgacagatttgctaataagataaaactatgatttttccatcaaaaatttg
ttataaacatatcctattttattttggaggaaaagaaaatagaacttacctttgaatttg
aaagaagaaaagatagatttaattgctgaatgttaaagattgtcttcgatttatactatt
agaggatttaaagggagtagataagaagacttttctgtaaaatctggtgactgagataca
ttttttaacaaaataatgctggatagaaaaaaacagctggaggagttgtttcattagtaa
tttatcagagtttgattgttaactattacgatgcaatttgtaagtgttctttgaatttaa
aataattaatcaacatagctcaatgaattgagtgagaatttcttttcaaatataattttg
ttaaaccagaccttcctgctgctttttttccaaggtctttaaaatggatacccaatattt
accttgagtttagtttaattacatgcagaaattagaaaggaaagtttaatacgttagtaa
tatagttatatattctttctgctattaattggtcatttttagggtagttagttttagaga
gatcatctagtcgtcctcattttactagtggcagaattgaatttcaagtttgtttgagac
agttaaatctcaaaatttaaattattgtataaaaatgtttagatgatcttatttagtttt
gcatataagttcttaatacttacctgttttgaataaatgtatttctaatcaagactgagt
cctgtcaactattcagaccaaactttataaatagcagtaaaaaattagaaatattataga
caattttgaaggcatctgaggcaaacttaaggaataatgtgattcttgaaattcaaaaca
tttgaacatagtatttctatgtctgagttttttaaagctttaataaagtatcacaaccct
ttttataaagaaaatttaaactttacttatgtcaccaaagaccagtcataggagaagtgg
gacttgtatgttctaaggaaatattttaactttatttgtatatattaacaacttgcagtt
ttgtcatggagattctgttatttggttgcaattagcatttctgggataataacaggcgtt
atataggagggaggaaacttactgtaagaatagcaaataggttttgtgtacctgtgatct
ctgggaagaaaggtaattaattgatgtttgaaatatgccagatcatcattctttgtacta
gaatctattattaataagtatctatctttgctttcacttttgtttttttccttttatcct
ttctctctctcttttttttttttttgtagtaccactgggcttgtaccatgttcagcaaca
ccaacaacttttggggacctcagagcagccaatggccaagggcaacaacgacgccgaatt
acatctgtccagccacctacaggcctccaggaatggctaaaaatgtttcaggtgacatat
ctaaacatagtacttgaaatgcctcacttaattgctgcaaaataagggattattcatccg
taaaaaaagaaggaaacactgttacatagtatactgtggatgaattttgaaatcattctg
ccaagtgaaagaagccagacacaaaaggccacatgttgattctctttatatgaaatgtcc
agaataggcaaatccatagagacagaaagtagattaatggttgcctgacaatgaggggag
gagaaggactaactgccagtaggtacagattgtccttttggggtgatggaagtgttctgg
aattggatagtagcgatggttgcacagcaaggtgaatatactgggaagcactgaagtata
tacttgaaaatggtgaatgttatgtgctgtttatttcaatttaaaaatgctattaaaaac
aagggaactcaatttttgaatgtcataattgagaaagtccaatacttatttgtgtaactg
tgcactgacattttttttattgctttttaaataataagaccttccttcatcttctccttt
gtttgaatttctgggttgtctagttttctaaactgcattgcttgtaagttaggcatttaa
gagtaatttaagaaccattctagctagagttaatacagcttcttttcttcagtgccatgc
ttgtatatggcttaccatgcttgaatcctgattactatattttaatagtatttatattct
ttggatgtgtacagggaagattggttacaaccaaaccctgaatattatgaactttataac
ctaagatagtgatattacattgatttatctcttcttccagtaagataatgcatatacatc
aagtgtttagcaaaatacatggcaaggattaagtacttactcaattatttgttgcaaata
ataaataatataaattatgtaaagtaaaaaatatctgatattagcctccatagtaatgta
aaaagaatatactaccctacctctggctgcttgaatggttaggtatttaatcaatgcata
aggcaataatcgttggtaaagatgatactttacttgacttttagtgtttcagatttttaa
ggtgttcactttgtaaaccctaatattttaaatacctagatgaatatgagataaatgtgt
ctatttttttcttctgtaggtagaaaactaacttggagcagtattgcttttaaatggttt
tttaaaattgttttctacccttcaggtgatttctgcaaaactaggataacctttttttgg
ttggtaactgaaagatttaagccttgctctcaacagggattagtttttccccgcgttgag
acaaaagtgtctcaagacatttttcatttttataactgaggggatggggcgcaggacact
gctggcatctagtaagtagaggcaatacaaaggacgatctccatgccaaagaattatcca
gcccaaaatgtcaatggtactaaagaatctgggttagaggaatgaaagttagattttatt
gttattaaaatgttataagccactgtaattacaaagaattttttttaagttttcattttc
atagtcatttgttgattaaacttaccttttccccagggattattcctacccttcctcctg
agttattttgcctctggttcatgatggtttgtaaaatgttaattaatttttattttttcc
tatgcacaccctcagaattctaaatttttaattttgtcccatccttttaaaaggaaattt
atccgtcaataagaggcgaaaataaaactcggacacttgtatagccttaaaaaaataatt
taaagatttttatcatctctgattaagccaaagaggtaggaatatggttgtatgttgcca
tgctaacacatgcatgtagtgtgaacagcatatatcattctgatttcatatcacatgatt
tcactgatttcatatcatttttataagaaaataccaacatgattgaaaattacactatta
gcaatagttttatcatcattgtagataacctccaaaatgcttattttatatgaaaaggct
tatttcttttcagaaggtagttcagagtaagatttactttttctgtggtagggctcattt
tttccaacgttttggaatacctgaatattctttgcttgctatctaggatatttctcaatt
ttaaagggactctgtagtattgatctataggaagaggcatttcagctttattttagtcag
gatattttagagagttcattgtacaccgtttgcttgtttagctagcttatgctttttaaa
taaaatgtccaaatgttaccgcatgctgtatttcatcaaatctgagacaccatagttttt
ttagatgcattccaatttcggaaatgttaatgtgaaaaaaaaaaaaactgtattcaataa
gctaaagaatgttagaatagcttttggtcctgtaaaaggagtaggtgcagtgaagaaaac
atgttcttagttaacaggaatagcatgtggaattacccctgcaggtatagtattatatac
atttttttcagataaatattctgtagcaaagaaatctgtagcaacaaaaggtcaactgga
caggtagatcaggaccttcatcttttgagtaccatgtctgtctctgacacacccaaggcc
tcattgttgtacactgccctttattttcagggctggtcccagagtaactagaaatgattc
acttgcctaaaggcttactgtgctttaatgtctaacttgtagagatataatttcataata
aagtggggagtcatgagttgacctatgtgagagagttgagggataattaaactggcatgt
tttctacaaatatactgtgggggcatttcatgatgacttttctgttgcataataagcagt
atgggagagcttgtccccttactgttctgctattgctcctaaggcagtgatacagcattg
ctttggtccagttccatctattctgacaaagtactctgtattctgtttataaattgagta
tgaacaaatgatattccattttgcataaaagaggactgaaacagaatgccactagaaaat
agaagtgcaaattaaagacaaaaaccaaaaaaaaaaatagaaagtttataccatttgaat
tttcagtagccattgatccactttgtatggcagtcagattgatgcttcagttaatttagg
gaagcgagttggtcatttttttggcttgtttatatcattcaggtgctaataaatggtttt
tcacttaaacttttcagactgggtaaagacattaattacatgaacattatcttatattga
aatttgtattcagaagcatcttgagaatagcacaaatgacatagaaggacaagttcttag
ttcaaattttgacattgtctcttaaaaacaataatttcattaatatataaccattcttgc
ctttattttcacgctctataaaacacataacattaaaaagttgtcaaaggggaaaaaaca
tgtacatctgcttaaagttatacttggaaatatattggagactcaataaatgttagtttc
ttcccttggtcaagaaataatcttagtggacctcagaaattttctcagatctaaaaaggg
accgttaacactagcttcacagtattgctatgaggatcaaagataaagtgatatggaaga
actttaaccttacacagatgccattagctttacattattttaattaaagcatattggaat
taagctatttatgtactttgaaaagatgttatactaaaaatatttttaaatgataagtat
aaaatacatgtgttttgatattaaaaatggagaaaaaacttctccattggaagatttaat
taggtttcatgggaacacttagaaattgtacatgttctcaaatctactttatttacatct
tatttgctaatcaacaaaagattaaaacctacaggatagtgccaaaattgaatttattat
tactttgaaattgtatttgggacacagtgatttaaaaaccttgttttgtgtgaaagactt
gaagaaagtcatgtatttagctagagaagaccaggagaaaatgtgatgtctgtttttaac
attttaacgacttttgtataaaagtaggaaaagattcattcttggtttcttcagaaagta
aaacctgaattatgttctttgagtcagattttggcattaagttaaacttagcaattagaa
ctggtccacagtagaatgggagataataagttactcgaatcctggaactgttcaaacaga
ctggataaccatgtgctagaggtgctagagaagtgagttctttaccacatgaaattcagg
tgcagtactacccaaggtcataaaacagtctatctaattgagacactaaagatcatccag
accagtggttcccaaatgctgctttgtgagtcatctacatcaacactggatgagttgctt
gtttaaaaaataaaatgtcttcagccactgaatcagactatctgctggcgggttttagaa
ctcagtaattcttgctgtccttgatcggcatttgggaaccactggcctggaccaagcctc
tcactgtgtaggtgggttctctgagactcagactcattaagagacttccatgttgttcta
cagtgagtttcctggtgaagcccaggctcttgattgcagtttagtgctccttctactctt
ctctactacctctgtgccctaatttacctgaaataaaacaccacttccatggggaatttt
caaatttgcatttgcaattttgtccttaaattaaaatttgcttctgaacctggaatgttt
ttcctctccctttcaatgtgttccaacatggctaccatgaaaggccaaaggaagtacttt
ttttttttctcttttctgagacaaccacttttcaagtagcagtaaaataataattaaaag
aattatccaacactctgcttttgcagataaactaaggcctgaagaggttaaattgtaacc
taattacaacgacagtatctcctaaaattatttcctgatccccaattcagttatctctca
actattcttcgttaattgttgttgtaggttgttatttcatgtacacatgcatgcgcgcgc
acacacacacatacacacgtagatagtcattagctgcaaggttttcatgttaatgtatat
gtcccttgctgctaaattgtatatatacaaaatttgaaaccactcttggagccagaataa
gcaagcaagtcctagtgataccatcaaatataaagaatagaaacactctctagagataga
gttgagatgatagcattaatctagagagagtatttcctgattcttttgggattcatttat
ttattcagtaaacatttattaagcaccttttctgtgctaggccctacttgggtgtggtat
gcaatagtgaataaaacagacaaaaaacactttttcctgaaacctacattctagtcagaa
ggggatgaggtggatgagtttggaactggttgcaattttagagaggttgctcagggaagg
aagtaaggaagcaaggcatgtgggtatctagggaaacaatattccaaggaacagcaagga
aagatgctgtgaagctgagtgtgcctggagtgtttacagcacagaaagagtgtgtatggc
agagcagagtaaataaaagggaaagtaatgggaaagtagtgtgttgtaggccaacctaag
gactgatttctactttgggtgaaatggaaagccattagaaggttttaagaaggtttaaga
ctttctgacagtagcactgagtacagtgtggagaatagaacgaagagccgaaggtagaaa
gctggaagaccacttagggagttacaggaataatctaggtgacgcatactggtagctcaa
tccagtgtagtaatagtcagattgtggatatattttaaagtttaaggctgtgagttcatt
tggatcaaaagcatctgatggtgaaagagagggaggggtcaggaatgaccaaagacttct
tggcctgagtaactagtaaaatagagtttattttactgaaatgaggaagactgtggaaat
acagatttggagagaaagatcatagacatgttaagtttcaattacatatccaaaaataga
tgttgaaaaagcaacaacataatacaggtctggagttcaggaaggactccaggctggaga
gaggaaagctgtttttatggtccagatctttgtttagaatcaccaaagagatgtgatttt
aaacgataggacagttgaatatgtgtagtcagtggaaacagtctcattttctattgctac
tcgtctttatcagtggtatccttttttttttttttttttttttttgagatggagtttcgc
tctgtcgcccaagctggagtgcagtggtacaatctcggctcactgcaagctcctcccggg
ttcacgccattctcctacatcagcctcccaagtcgccgggactacaggcgcccgccacga
tgcccagctaattgtttttatatttttagtagagatggggtttcaccatgttagccagga
tggtctcgatctcctgacctcatgatctgcctgccttggcctcccagagtggtgggatta
caggcatgagccactgcaccgggcctatcagtggtatactttttaataagataacacgaa
gtaacatctgctgagtatatagataatttaaattgagacgttttaaattgtctctggttc
ttattaaagattctttaaaacaaaataaaaatacattgtttcaaatattgtttaaagtct
tagatatgttttgttatacaggcataatttttttccatgtactttttcttaaatatatgg
aaatttctaacacagtttcaaatatttagcttaggtctgaattagttgtttatcatctgt
tcatctgttctgtcttctttaatttctacaccctatcctccaaaagccttgataaaagat
atgtctcatatattagctttttttttttttttttttttgagacaaagcctcgctctgtca
cctaggctggagtgcagtggcatgatcatagctcgttgcagacttgacctcctgccacag
ccccctgagtagctgggactacaggcgcattccaccatgcctggctgatttctaattttt
ttgtagagatggggtctcgttatgttgccaggctgatctcaaattcctgagctccagtga
tcctcttgcctctgcctcccaaaactactgagattacaggcatgagccaacacacctggc
ctaagctacattttaatcaagtcagcatagttatccatcactccaaatgagcatgtcctt
gtattagtgtccatgcattgtctatgatgtagatggagaggtatagcatattatcaaaac
ccacctcaaatcctgtgagggatgtgaatcaataaattaatgagtttgttctattgatac
caaatggagagagatgagtggagattgatcactatcagttatgtcaaagaggtaagtttt
aaaccagtgtttgaagttatgtgagagaaaccaaaccaaacaaggtaattaaacttcaaa
ttaactgcttttaattggtcacgtgtgtacatatacatgtttatgagtattttacatttc
tttgtataataaatcccctgaccaaaaaagatacaatgtaagactcaaggtgaatgttct
ggatacatgtatttgtagaagcaaaaggaagggacacaactggttggaacctgtatatct
ttaacattaggtggtaaaatggaatatactatggaaataaataccattgaaatgttttat
ggtattgccaatttaatacatattttatgataagaaagaagagaagaagttcattgtaga
ttccacatgggtaaatactttaaaaagtattccaaatacagctcctcatatccttaatat
atctgttgtcaattattatatatttcatgtcttggatcaagttttcttgtttggacaaga
aatggaatggcaaaatccagatctttttagctgaaaacctcacctattctctattcaccc
acttctaaagtggacacaataattcttaaaatggtacttaaatcagtatagatttgcaga
aacaaaggcctgcttaagaatgttctttcatttaagtattatgaggttaattaaatgtgt
gaaattgtaacattttgaattattcacatggcattttagtttttccttttaagaaccatg
gcttttctgtgcaggtgttcttcactgggataatgttttaagataatcccctgatgtcat
ttaaaggaactcttatttctgaagcatgattgtgtgtttttgtgtttttatagtgtatct
ctttcttatcctcttttggcctttgtttatattctcattttaccactatcagctactatt
ttctattgttgattgtttttattgtttattctattttctattgtgtatttgttttcacat
aacaaatcaaagcctgtttaatttgtggaagacagttgtcaaatgtgtaatatatccagt
tactaatttttctctgattgatgctgctcgtctcctttcagatgagtgccttattaaaac
catctaattttatggtgcttactagaaagaatgtagagaaattgtcaaaattttctatta
tttactcagtaacccaaaatgatgtttctggaataaaattttggcttctcagttagtgac
ttggtggtagatgaacatataactattataataatgtaatgaaaaaatatttttctgtca
aacatgtttttttttaaccttttacgaaataaaagttgttggtaagtgatgaaattaaat
gccttcttcaatgccttgtggaaaaaatgtcactataaggagccactttttatacagttc
taccctgatcaactatggttgtgtgtagatgtgagttttcctttataagcctgtaatttg
ggacatctgttaaaacaataaaaatttctaattctctttgatagagctggagtggaccag
agaaattgcttgctttagatgaactcattgatagttgtgaaccaacacaagtaaaacata
tgatgcaagtgatagaaccccagtttcaacgagacttcatttcattgctccctaaagagg
taaagattatcaattacattcttcattgggtgttatttgaatatataactcaacagctac
cacctttaaacattgtcatgctaattatggtattcggtattgtcagtttgacactcttaa
ttagtggtaaagagggctgtaaattgataaattgccatgacttaataatgtaattgtcag
attaaagaatgcatgtgaactaagtaactcctttccactatttgaataagtcatatatgt
gagagtaactattgagtttcactaatttgattatttatttaccaagatttttttgtcaag
ttatttgaagcccaacctttagttatcttggtatattcatgatagtagattttttaaaat
tctgaccatattaaaatagcaatattttttctcttattttattatatatcttaagtccct
tcaagaccaataggtttatgattctattgaattctagttctctggtacttaacattgtgt
aattattttaaatacaaatattataatttcacatgtgtatatttttctaatcattctcct
ccccatctccattacacaaatacagatttcccatactaattacatgctaacagcacttct
ttgggaattatttgtgcctatttctttttatatttttatgtaagctgaaactatcataga
ctgattatatgggcattcataactataaggcatagtttagatatagttcctttattgata
ttatagacctagtaattttgtcagtgaacagaaataatataacattaaataatcagcatc
accatgatttatattttcataagtaatatgtatatgcccctagttatatgtcagctaaga
agacaatataaaatactaacataccatttcaaatgatcctgaaacttcaaatttgaacag
aaaaaagtcatttttttaagattatcattctaaatatcctaatggtgttaaagatagtcc
caattagaattgcacgttggatacgaataaaatagttgtatcatatttaatgcatggaac
taagcataactccagagaaaagcaaagtggagaaaatcttttcttcctaaaatgtgtttt
ctactgagtgctttttaaaaatgactttggcccagaagaaaatttttatttttgctggct
cattctaaaactgaatcctttgaatagatttggcttatgaaagtgacgtcagctctcttt
gcttccagaagggcattaataatgggaaacacttacataagtatttgactgaaagtgatt
ttattcacctttgatatattctctgtagtttacttagtgatatcctccgtattttagagg
ttatcatttttgttaaaatgcaggctttctcatagtctcattttttctcttatttctaat
aatggtgaggtttttggtttgttttgctttatttgaatttccataacttcttaacagaca
aaatgttgtgtaagatcagaatatgaaagtattttgaaaaatctaagaattaaaaacaaa
taatcaataaccagggtaaatccagtaaagattctctcagtttaagtgttcttttaaaga
ctgcctggggaaaaataagtgaaattagttatataggagactaagggagagccttggttt
agcatatcatctgtgtttccctcttgttttcaactaccagaaaaaaaaattcctattttg
aaaaatgctttattgtctaagtggataaataatgatatagaaaacattatcttctcagaa
acaaagtattattatttaaaacattattaccttcttgtcaggaaatggacttatttttct
tataattcctttggccatatctcaaaactatttacaaacatttactttattaatctcaaa
aagcttgacagaagtctcactaatttcacactagaggcttgtgatgaatagcattgtctt
tataagtgaataccaagaaaatcataatcatacaatagatgatctagagcaaaacctgaa
attcttaatagaggattagccaacatttaaaaataattttttttccttgactagtttaat
ccactgaaatacagcaagtctgggaaaatattggctaccttttaagtacaatgtggttgt
cttttatataggttatagagaaaaataaaagcaattcttacagagttggacaaggaaaag
gtaattatctgaaatacgtttcaattcaagagcttttctctggcagaagtgatctttagg
aagaaatactaatctgggttttacctttaaaacaaatgaatagaaaactgcattaactgg
accatttgacttggctgtaagaaacccataactccctaccatagcggttggacagacaag
agtctcactctgttgcccaggctgggatgcagtggaatgatcatagctcactgcagcctc
aaactcctaggctcaaatgattctcctatgttagcctcttgagtagctaggactacaggc
atgtgccaccacacttgggtaatttttttgcggggtgggggtggtggtagggggggatgt
cccttttatgagagtactaattccattcatgaggactccaacctcatgacctaattattt
tccaaacacctcacttcctaataccatcacattgagggttaggatttcaacatgaatttg
agggaatatggacattcagttcattttagagccctacatgacaaaaccttgcttcctttg
agggaactggtatacttcctcctcataatacgtagtaagaatcatgcaactaattgctat
tctctttttggcttggcagtttaattcaaaaccttgtaagacataaccttgttgacagcg
tatttgtgctctactttttctctagtttaaaccttcttttctgattttttaattcatatt
tttttgttttgctttgtgtattttctgtattgcttagatgttttgatggaactggctaga
tgtaaataggcaaacatttaataagtcgaagttcttggttgagttagtatcttcactact
tttaaggaaagtgtgccagtaacctgtagagttagcacttcaactttctttagcgcctta
gtatttctttatatttctctttaatgcactggactagatgatctcataatcactattaat
gctggaaattttacctggtgatttggggatgtacttttgaaatctaagctgctctggatt
tgctgtttcctagtctttaagatacattgactatttctgtaaattacctgatttgtactt
cagttagataacgaaagcaaagaaaagaatagactttttctgtttttagataaatatttt
ggtcattaattagcatctcttttgcacaaatatttttatgcctgatagcttctatgttaa
attacaattccacatagattagtaatggtaacaccacattactgctatactgacacaaag
cgaacttgcctaggctttgattgattggtaattcccaaattaagtagcttttggagtatc
tgttaagtacaatattgagactatatatgtgtgagtattgttgaatatagttacaccttt
ttattttaaatgactcagcttctctacataatactggtcttctctgatataccaaaagta
attatttcacaaaaagtttttcactatcattcatcagtagtagtattaaactgaatattg
gctggttggagtattttgtatattgaatatactcaatacatatgtaattaatgatgagtt
gttgttgttgttgttgttgttgttgttgagacagaatcttggtctgtcgcccgggctgga
gtgcaacgacgtgatcttggctcactgcaacctccacctgctgggttcaagtgtttctta
tgcctcagcctcctgagtagctgggattataagcatgcaccaccacacctggctaatttt
tgtatttttagtagagatggggtttcaccatgttgaccaagctgatgtcgaactcctggc
ctcaagtgatccacccacctcggcctcccaaagtgctggtattacaggagtgagccacca
cacccagcccatgatgaattccttatatatgcaaagtcattgggaaagcataatttatat
tatatactttacatttagcatcaaaaaataattaaaattgtgcttttactccctgtgtga
cccttgaccctttaacggataatttatgttagttcttgtcatttatttcatatgaaaact
gaacttaattgctctatttaaattgttttccctagtatcataatttaaaattttatgtga
tatgtgaggctacttgaataatcagtcagaggctataattccccttctttcttttttttt
ctctttatttttcctttttatttatttgaactgacacactgaaaaaaaatcaatagttta
taagtattataaacaaagtttttaaatcagtggtataccaaagatttagctcaaatatac
aactcttatgtggcccatcaagaagatccttataagtaatataaggtaatcagaactcaa
ttacttttatatgtctatggaatggtttaataattgtccccctttgtcatttacagtcaa
ggatgtaagtgtttcagaagttcattacaataataaacactttgatttgaagactgttaa
ttatgatatatatctatcaaaaatgatctggagtcataccacacagaatatatacaaatc
tcatttcttatctgtcagggaagcatttaggtcctatcttcagtgaatttacattgtagt
aggatagagaaaaaagttaaacaactaataacattttttttaaaaaattgagttgaggct
cacagagtaaaggcaagagcagagacagagaggtttaataagtagtccttgaagagggta
aagagttagtagagtgaagttcataaaaaccataaggtgtgtgtgtgttttttatctggt
atatatttgttaggactgaagttactgcctttcataattttagaaatcagagtgcagaat
tatatttatcaagtatctcatcctgtggagaataattaacttattatataatatatatat
ttatttaaatatgggtattatactatgcatttttttcttagttggcactctatgtgcttt
cattcctggaacccaaagacctgctacaagcagctcagacatgtcgctactggagaattt
tggctgaagacaaccttctctggagagagaaatgcaaagaagagggtaaggttcaaaatt
gcatagagaatatgtttatactttgaaaagcagagtgattctgaaaacaagtagaaaaaa
taaaaataatattcagctacttatgataaaatccacagattattctacacattcagatga
gccgaatcttaacagctaacccctttaacaagcaatataactaatagcaaatatatctga
tttgcttcctatcactagtagaattaataaacaatgaaaaagtcaagatgaggatgactt
aaaggaaaccccgaaattatatttccaattaaatatccaaatatttagtattaattcatt
cttttcataaatgtttattgaacacctcctatgagccagacatttcttcaaggtggggga
tacagcagtgaactgaagttctcttttgtcatggagcttatgttcttgaaacctgttttt
ttccttagtagaggagtagttatcctgtgtattgggagaactgttttttttgtttttgtt
ttttttcccagcagacatccagaaaaatatttttgaaaaaatataaaaatactatgtgcc
tcagaagcaaaatattttagaaagaatcatagagcatgatgtaggtactctataggtgat
tattcccttcatggtagttatagtcaagtaggaagcattgatggttagtccagttaataa
aggtttgaggtaaagatccagggtgcttggtttgtcatcctgcttttgacctctgtaggg
cttggattaatcagtgaaatcttcattcttaagtatttgcatacataaagtggaattaat
ctgtgccttcccttcttccatataatcattttttaaatgatcttttaaaaattgtgataa
catgtatgtaatttatcagtttaaccatttttatatgtacagttcagtggcattaagtac
attcacattgttttgcaactaccatctgtgttcagattcactaccatctgtgttcagaac
ttcatcatcttcacatgaaactccatgccccctgttctaactccccattattcctctcca
cagcctctggcaaccacccttttactttctgtctctatgagtttgactactctaggtacc
tcatataagtgaaatcatacaatatttttccttttgtaatcagcttatttcacttagcat
aatgtcttcaaggttcatccatgtcatagcatgtgtcagaatttcctttattccattgtg
tatatgtatctaatgctacattttgtttatccatctttcgatggacccttggattgcttg
cacgttttagttactgtgaataatcctgctatgagcatgggtgtacagatgactgtttga
gactctgcttttaattgttttggttatgtatccagaagtggaattgctagatcatatgtt
aattctgtgtttgttttggggaaccaccagactattttccacagcagctgcaccatttgc
acaagcattccaatttcctcacatcttcaccaacatattttagaatcattttgagaaatg
aaatagtgaatacaaaaatatgccatatagaatgtaaactttacattattgctacatact
gtgataacagtattggaaatttgacaatgtcttgcatttcagtggttttgagcagagaga
tggaatactctttataaagatggaatactttataaaatgtataactgttattattggtga
aggcaatttactcttgaactgtaacactttagggattgatgaaccattgcacatcaagag
aagaaaagtaataaaaccaggtttcatacacagtccatggaaaagtgcatacatcagaca
gcacagaattgatactaactggaggcgaggagaactcaaatctcctaaggtaactgaact
ctgcacaatgtaacagaaaccgttaatcagtgttagtacctagtgtactatttcacttgg
caaactgtataatatgacactcctcttctgtagaaagaaaaccgtatattttaaaataac
tgaaagtattaactgacaatattcttttattgctaatttctctaatctgacaactaattg
tgcttgaataaaagataagttaaattgagtatctttttatcttacgtaaatgaaaatctc
tcaatatcctattgaattttactgtgtcatacacattctacctaagaatttgctgcctcc
tctgatcacatactccaaacatgattttttgaggagtgtctgtgttttggatttggtgac
ttttgtgtgtcttatttttacaaattgaagcataattctggttttctgtctaataacatt
tcaaatacataaagaatggtaaaaacacttgaaattattgggcgtatttttacaactgga
aaaaaccaatttggggagacattgttatactaatatgtatatattatgattttttttgtt
ttaagaatgctaataattgttactctttgtggttaatagatgcttattttgttcttgtgt
gtcaaagttgtaaaaattagaggcatattcttctggtttgaacagatggaatagattatt
gtcaaatacattggtgtttgaaccatctttatgaggtcttgatgcagtaaaactttaata
gttgatgttttgagtataaataatagactatggaaacaataaaatgtttaattctggatt
cgtcagccaggaaaaataatgctttatataaatttaaattctagagatagactacaaatt
actgttcctgtttatgccttcatttttctcttcaccagtattttaaagaacataatttaa
ttaacatatttctaatctgcacatctttcttataggtgctgaaaggacatgatgatcatg
tgatcacatgcttacagttttgtggtaaccgaatagttagtggttctgatgacaacactt
taaaagtttggtcagcagtcacaggcaaagtaagtttactcttcctacacttcacaaagt
cataattttgtgtatatcattgcaagtagcaactggaaaaaagaaaacaataattcattt
ttgattcttagttatttggatagcaaatatcagagctcataacttagcttttttccccat
atttatgaaagccaatggcatgaggatagtgatgcttctcatgatagaggtttcattaat
tttttttcatttacatagtttgtctatctttgtgacagatactttaacattttcaagggt
gctttatgtttttcttggctcaatttttccccacttttatttactatctaagttatacct
cattactttgtaatgtttttcaaaagtatggttgagggatatttatttttccttataaac
tgctttgttgataacctacctgtagtttagaacttaataaatcatgttaatttatactta
tctcaaacccttgtgagatgagagtggacaaggcatttaaatgttaagtttgaagaaaaa
gcccaagcaatcagcttgactgtgtaccatatcataccttaatgattctctagtgaatat
ttatttttatctgtccagtagagaatattttcatgaattaagtaattgccacctaaaaaa
taacagaagagagtatgaaaacaaaaaaaaatccatgtcttttaaaatgtcttttaccat
gttgcttcttggcaagtatcttataaatattccaagtcagatgttaaaattttttaaagc
agctttatatttccatagtgtaatattagtttgggatttattacagggctttaatgtctg
tctttacttgtctagaagtagcttcactggctcctaactactgccctggggcagattcag
atgaaatgtggtatgtagccacacccaatactgttggcatactttcattgaatctattcc
catggaagaaacccagttgcaatttcagtattatttcatcaaagaggccaggccagagct
atcataactgctttcatgtcgtgtttcctttttattatatcatgttttatatcatatgtt
tgctttctacatactacatacacaattgcttgtgcttatataaatttataatgcttttga
ataaacaaaacaataaatagtgatgggatcattttatacggatgtaatattttctgaaga
gccaaacaaattaaataagtaactttactccaaattttaatgagctattaaattttattt
aaatcacttttcctttctacccaaaagtaatcatcttaagtgtttttccagtgtctgaga
acattagtgggacatacaggtggagtatggtcatcacaaatgagagacaacatcatcatt
agtggatctacagatcggacactcaaagtgtggaatgcagagactggagaatgtatacac
accttatatgggcatacttccactgtgcgttgtatgcatcttcatgaaaaaaggtaaggg
aaaatcttgtcatggttgggacttcttcctctattattggtgaatttgggccctttctgt
gtagagttactgaaatagtgacagtgtgctcttaaattagatttaattattacacttcag
atgtttctattctaggaaagccatagcgttttgtcttaaaattggtgattgtctgtaaat
ttcctttctcatttgctagtgttgtcagcttcctgtttgaaactctctactctctaatct
ctaatcctttttgttgttctgtatctttgatgactcatcatattcaagcagaaatcatgt
tttctctctctttttttgatattcttatttccgcaagtgggacaaattcaggcttcaagc
gtctgagcctttttaaattacctacccaccttacacccaccctatctctaagtctaggca
aaagctgaggcctgtgggtaatttgttctctcattctgaactattgtcacagctttctaa
cttggttatcttatgttttatacagctgacatatagtccttttataattatgatagtgtc
actgcccagttcttaagtctttttgagcctactgttgactaacgaattaaggaagaaatc
ctcgatccagttttcaaaatggtttctgccaaactggactttttgactttctcttctcag
tgcctttgctgttgcttttatctcctcctgtaaagccctcctattaaaatcctatctttc
tattatcgtcatcatacccctgttaaacaagtgtgatttctacctcatttgtcaaaaaag
gtctctctgaaaggtccttacagtgaatccatggcagtctttacctttaattctgtctat
cgcatcttctttattaagaacaattacattaagcagatttcacaccagatttgtaatatg
tgaaaaggagtaccttttcaattgtcatttacctctactttgaacagtgtgctatactgt
taaaatttgggggatgagtatcaagagaccttgtattccagtttccattatgttctctca
acattaggttctcaattaaagggttctctcaaccctttaattatctacttttttaaaata
tggaaactgtggtatttctttatctcttcagaatgacataaggatacctgagttttcagc
tttgttttgtaatccaaaagtgattacagtatttgtatattgctctggttttcagaagag
tatagcgctgatgttgaaccaaatgtttaagggatagccattgtttttaaagatgtacat
atcttttaaaatctttatctcttaatatttttatagcctattgcatatttataaatctaa
tcattaaaagcataagcagcctgtgtttgaggacatgtctaaaactggttaatttttact
ggaatgtatagtgtaaacacaaatctgcagctgtttaaattccaaattctgacttgtttt
gttgtataaactttattgaattttttacctaatcacaagggaatatatgcattcataatt
gaaattttggaaaatggcaaagaacataaagaaaatttaaattctttaaaccactaccac
ctagagttctcccattaacattttatacatatgcccacattaacattttatttctaaaat
caaaatcttattgtatatatttagaggcagcttctgctgctttttaaaaaatacagtaca
gtggttttcaaactcagtcccacagaaccttaaggtgctgaacctaaggtccccaggtca
gggaaccaagcatgcagcattctaggcttcccaacttcccattcccttattatgtttata
taccaaggttttacataggatttcttttgaaaatggttgttgctgtgtaaaaaaaaaaaa
aaaaaagctaaaaaaccttgactaaatctaccatgttttctcatattattaaaaattcta
aacgtgggtttttttgttttgttttgtttttctgtttctccctctgcagagttgttagcg
gttctcgagatgccactcttagggtttgggatattgagacaggccagtgtttacatgttt
tgatgggtcatgttgcagcagtccgctgtgttcaatatgatggcaggagggttgttagtg
gagcatatgattttatggtaaaggtgtgggatccagagactgaaacctgtctacacacgt
tgcaggggcatactaatagagtctattcattacaggtaagatctcttatctctcccttaa
atgctctcctgatgaatcataaggttgttttactcagataatcactgtcaaattgctgat
ccagtacagtccaaaaaagattaatcagattattctgctgtttttatatacgttaaaatt
ggcatgaagaaagtgtaaatttcaaaattatttaaatgctttcaattttctgtgatctat
ttctcctctaaatacaaatatttgttttgcaacctgacactttggggctctaagttttgc
tatgttaattaatggttaaatagtctcataggagagtaacctggattttaagaaactcct
ttgcagaagattaatatggccaaaagctgcccctttgctttgatagctgaaagttgatgg
accatgtaattgtgattccatccccattcaaagtggaaatagatcctttctgcataaagc
actctactgtcccaggcccactgatttgtagtcataaagaagtaaagttgactaaaccga
tttcttttttttggtttatatttcaagcccatatccttgaaaacattgccatattttgag
atcattaactagatgaataggaagtttagaatatgaatcttgttttatttccagaaaggc
aacatttttaatgtaaaaatatttcctatagcttatatagtttatttaccagttattacc
cattaatgttcagcaaattagtacataatcttagtactgtcaattctgttcttaggatat
ttgtttctttgtcaaagggatattgtccatgtcatgaaatattgacaatatgataaaaat
gtttgctaaattagaaaaggtgatgaatttggaggaaggggaattggctgcacctgtttc
tgatatgttcagaagcttaatgaatataatattctaatttaaataaactgtttgattgag
aaaagaggtagccacattattgtttagaaatgatagactgttattgacttttggtgtagc
tgggaagctggagaagaggtagtatgtagtttgcttttgatttcaaaatgccacctcttc
tgattccagatacaattatcttttggcacatttcctaattagcattaggttcttataaat
gaaattttattttacacacagtttttaatggaacttacttttgaacatcacgaaagttat
ctctagcccttttcatgccttaggtgctgataagcattccgtttatcataagctatgtca
ttagtctcagcttcctagtgggaagtaaaactcatagcaattctctcagtcatccatgat
atatagctaggtggggccagatgatttgaaaattaacatattgttcattttaggtgcttt
gttttcattttaagttgtttctgcatctagtttgaagctgcttggcataaagatgagcct
ttctgatagaagtgtgtgagaacatacattgtagagttgcttgatggcatgcactttatc
ctcattgccactttatcttagtacctcattttgttcctggcattacctgtttcccatcct
cttccccctttcctactaggattaaggtcagtaattgataggaagagtatccatacttct
attctaataaactgtgtcttttgtagtttgatggtatccatgtggtgagtggatctcttg
atacatcaatccgtgtttgggatgtggagacagggaattgcattcacacgttaacagggc
accagtcgttaacaagtggaatggaactcaaagacaatattcttgtctctgggaatgcag
attctacagttaaaatctgggatatcaaaacaggacagtgtttacaaacattgcaaggta
agtcttactcacctttcgttttgttcattaaaaatgagatcattattcaagagaaatatg
gagccttagcatcacaaaaagagtcaacctcatacagaatggttacttcagaaaagttta
tgaatattttcaccctgacttacagaactagtctactcttgaggaaatgttttaaactgt
gtttttaatatgtaatataaaattgtttaatcacttcctgcatgttcaattgactctact
taacctttaagaacagtgagaagaaagatcttggctatatcatagaagcaacgtactcat
tatattttttgattgaccattgcattttgaatttgtagtccaattttaaagggctacctt
attgaaaaaaacaacagcatccttgatagaaaatttatttggcctccacttagtcacgtg
cttaactggaatggagaggaataatagtgcctactaaagtagagaaagaaacaagtgaaa
atcactaatattagatgaggtacagcagtgcctcatgattaactttttctatttgttata
gcccttagaaagtgtttttttcagtggggccatctgataggcaacattgcagctttttac
tcattggttttgtctctcctcagagttttggaatagtgactgttcagctttgagtggttc
atggtggtggggtaatagatctcttagtgctcatttgtccatcagcagaaggtatgaagt
tgcactatctacatcctcaccaaggaatgctgtcgttcacacagccagaaaataagatgt
ttgtctggtagctaaccaagcttcttggaagacagacctcttgaaagttaatgtttcagt
tacaaatcactaccagattttcttacatccatgctgatcctaaaatactgaggacatggg
tttctaaatatgtatcatttcaaatgttgcatttattgtatgttctaaagtagaagtcta
caaattataatgtaactaactcatagccattatttctaaccagtaattaaattcttttgg
tttttgtctaggtcccaacaagcatcagagtgctgtgacctgtttacagttcaacaagaa
ctttgtaattaccagctcagatgatggaactgtaaaactatgggacttgaaaacgggtga
atttattcgaaacctagtcacattggagagtggggggagtgggggagttgtgtggcggat
cagagcctcaaacacaaagctggtgtgtgcagttgggagtcggaatgggactgaagaaac
caagctgctggtgctggactttgatgtggacatgaagtgaagagcagaaaagatgaattt
gtccaattgtgtagacgatatactccctgcccttccccctgcaaaaagaaaaaaagaaaa
gaaaaagaaaaaaatcccttgttctcagtggtgcaggatgttggcttggggcaacagatt
gaaaagacctacagactaagaaggaaaagaagaagagatgacaaaccataactgacaaga
gaggcgtctgctgtctcatcacataaaaggcttcacttttgactgagggcagctttgcaa
aatgagactttctaaatcaaaccaggtgcaattatttctttattttcttctccagtggtc
attgggcagtgttaatgctgaaacatcattacagattctgctagcctgttcttttaccac
tgacagctagacacctagaaaggaactgcaataatatcaaaacaagtactggttgacttt
ctaattagagagcatctgcaacaaaaagtcatttttctggagtggaaaagcttaaaaaaa
ttactgtgaattgtttttgtacagttatcatgaaaagcttttttttttttttttttgcca
accattgccaatgtcaatcaatcacagtattagcctctgttaatctatttactgttgctt
ccatatacattcttcaatgcatatgttgctcaaaggtggcaagttgtcctgggttctgtg
agtcctgagatggatttaattcttgatgctggtgctagaagtaggtcttcaaatatggga
ttgttgtcccaaccctgtactgtactcccagtggccaaacttatttatgctgctaaatga
aagaaagaaaaaagcaaattatttttttttattttttttctgctgtgacgttttagtccc
agactgaattccaaatttgctctagtttggttatggaaaaaagactttttgccactgaaa
cttgagccatctgtgcctctaagaggctgagaatggaagagtttcagataataaagagtg
aagtttgcctgcaagtaaagaattgagagtgtgtgcaaagcttattttcttttatctggg
caaaaattaaaacacattccttggaacagagctattacttgcctgttctgtggagaaact
tttctttttgagggctgtggtgaatggatgaacgtacatcgtaaaactgacaaaatattt
taaaaatatataaaacacaaaattaaaataaagttgctggtcagtcttagtgttttacag
tatttgggaaaacaactgttacagttttattgctctgagtaactgacaaagcagaaacta
ttcagtttttgtagtaaaggcgtcacatgcaaacaaacaaaatgaatgaaacagtcaaat
ggtttgcctcattctccaagagccacaactcaagctgaactgtgaaagtggtttaacact
gtatcctaggcgatcttttttcctccttctgtttatttttttgtttgttttatttatagt
ctgatttaaaacaatcagattcaagttggttaattttagttatgtaacaacctgacatga
tggaggaaaacaacctttaaagggattgtgtctatggtttgattcacttagaaattttat
tttcttataacttaagtgcaataaaatgtgttttttcatgtta

TABLE 2b
Mouse Fbw7 nucleotide sequence
Mouse (mus musculus) Fbw7 nucleotide sequence
NCBI Reference Sequence: NC_000069.6 (SEQ ID NO: 4)

gagcgagcggggccgccaccgccgcctctatcccagcagcgcggaagagacccgggtagc
tgcttggtggagcgacgctagcaccgcttcttcctcagttccgcgcctagccagccttcc
gcagctgcccgcctcagcccgaaggaggaagggagccagccacattgtcagcgccaccgc
tcgactctgaagcgtgagtggacccgggggagggggacattataaggtgacaagtgggag
cgggtgggtgctttgatgctggagttcggctgttcctcgggagggttggcagtggaaacc
tttcctttctaccaataggagaaagtgtcagtttgcaggagtaatgcaagtactcggagt
tttttcttagagcagctgtgtctccagagtggacacttgcacctttttggacactaaact
gagaaagcctatggccagctctgtcccatgccttttcctggcaaccttattctggcagtg
gtttttgtttcagggaaactcccatggattcccacgtgggaatcacttgaatcaaccctt
aatttttttttttcctttcctaactattgtttttgaagttgccttggatgggaagaccag
atttcattttagtgcataggggagtttggtgcccccaacgctttatttcccaggtctaca
gagtccgatgtgtagggtgcttgcatgtttcagttggctcaggaaaatccgagttcctaa
gatgcacaacccactgaaacttatttcagggagagcacttaataaattaccatttcgtaa
aagttgctgcagcgctctctctctctctctctctctctctctctctctctctgtgttgag
ggagagagggagtaatatgttttaagtggtagcacatttttactaggtttctactaggga
ctggaagggcatgcatgcagctgtttttatttggctctacttgtcgaccttgagacttta
tactcttttaggaaaacatttagcactctgagtgttaaattagaggtatagaaaagtaat
gctcacatatgtaggttaagctgaccatcctaaggcttaacgtactgtaggatttcatgt
agcaaggaacataggtagtagtttaagactggaattctggtctatccagacagctaaatg
tgggcagaataaagatgctctgtagaaatgactagaaacttgggagtatcaatttatcta
cccatccatccatcttaatttctccattgagggtcatgattaaatttgtttgaaagtttt
tcacatttcatgtttgggacctgttctcccctcctctttttaaataatgaacctccaagt
ttatcattctgcattatatagcaatgtatttctctttgaccagccttctcaatctttcag
gatacagtttgtttgaggaatgctaaaacactgtgaatttaccttaccttgcttttatag
attttgtttaatctctactatcatctgtcccaacttttcttttcttcaagactgtttttc
tatgttaccttggctgtcctggaactatctctgtatatcaggctggcctagtgttcacag
atatgcacctgcctctgcctcctgagtgctgactgggattaaaaggtatgtgccctgact
gccagccacgtcacagcttttttgagtagctccctcccccaaccctcatttctttggata
actttgccctctttttggatctcctagccttttttttttttttttttttttgttagatac
tttgttcttcatatgatttcatgagacactttaaattttgcttaagtgggaattagtatg
aaaatattgaatggtaaatttaactttagatttagaataaaacgtgtttttggatctgct
aaccttactttgctaccctgataaagtcatatattccagttttgaaaaagatgatatttg
agaaacttagactttaatagtttgcttcttggaaaggaggagagtggtactttgagtttg
tactgcatctaatcagaggacctgctttgtgtgtaaattggttttaaattatggaagtac
tttactgaatgtagtaataacaagacatttgctattttcagtcttttgagggtgacagag
acaaccaactacaaattgcacaaaactttaggaaaggttgaactggtgggaaggctctgt
gggtaaagctgcttgttgcaagtctaaggacgtggagtctgtctaatgcccagaaccaca
ggtggacagagaaactaaccatacagagttgttctctgacctgtgtcgtatgtgtggaca
tgtggtggtagtacagttgtagtagtagtagcagtagtagtagcagtagtagtagcagta
gtagtagtagtagtagtagtagtagtagtagtagtgtagtagcagtaaaaagaaatacat
ttcttaaaaatgtaggaaaggggacatatattaccattattactcataagatcactctgt
gtcagggaatgcagttgcacatttttattagggaaatactgattttggatgacccaggaa
taattggagtgtctgagccaaatcttggaaaggctctttcaggaagaaaagaacatacac
aaacaaaatgttaagggcttagcgtcagaataagtaaaagatgagatctaattaggaagt
tagcttggtaggaatgcctactgagggagggaagttcctgactagagttaaggtcagttt
gtgcttgtataagagaatttgaaaacagtgttccaaagaaggtagtttcaggatattggg
agaaattatattttaggacagacattatactggctttaggattttgtttcattaggagac
taacatttttatattatggtcatctagggggcagcacagaaggagacacgaaaaaggaaa
tgttttgaatgttttttagcatgatgctagtttcttatgagtaaatcagaaacagggtag
caggctttgctttggatgttgaatttggggttaagagctaggtatctgtacagttcatgt
gttagctcattgtttttcttttcctccctctctctttaaaactttttccactttctttct
cttcttttgtctctgtctcttgtgccaggctgaccttgaacttgttgtgtaccttttaac
ttgtaaccaccctgcttggacttcctgaatgctaaatgtactagtgtgtgctgccatttc
ttgttttgtagtgctggggatggaacctaggcctttgtgcatgctaggcgggtactttac
caactgagctacatccttaggacctaccccgtgcagctttcaagactgcaaaatagtttg
agtaatatgctgacagtgtaggtagatgggagtcatttttaagcctttatatttctgaga
ttactgatacgaaattaagagctgaaaaatgaatttaaaattttgggacctgaagcagat
ttgtgttggtataagcacagctatcaaaaggagagacagtccccagaagatcagcttcgc
aggtgagtgggaatgacagttggaaagaggagcagtctgtaacataatgaccattcagtc
ccaggacatgggattctgttttagaagtagttaaaggttttcaatagcacatttagatcc
tgagatagataagttcagaaaacataggaccttgttagggaacttataaataaaatggaa
ggccattacagtttccctgcattttataatttttttccccgagacagggtttctctgtgt
agccctggctgtcctggaactcactctgtagaccagactggcctcgaactcagaaatacg
cctgcctctgcctcctgagtgctgggattaaaggtgtgcgccaccactgcctggctcatt
attataatcttaagcttggtcttaatactaatgaagtggggctaaacattgatgaaaggg
aaggctgtttctcttacttcagaaatgtgcatctttgtgtccctacaatgtgtgtttagg
ggggacatccctgtgttggtacagaagtattagggatacttttttgtataaacttaaaaa
gagcctacaatttaggaatatttaaacaacacaaacaaacaaacaaacccaagctagata
atgatggcacatgccttttaatctgacccaggtagctctgagttcaaggccagcctagtc
tacagagtgagttacaggacagccaaagcaacacagctaaacctggtcttgaaaaaaaca
aacaaaacaacaacaacaaaacgaacaaaaaagactattcaggcatctttatggttagct
tttggtattggctttgataaggataaaagattgtgaataaaaacacgtaatcattatgat
cctttgtttgaattcattgtttttctttggcttcttaagagagcatccagttggcagtaa
tctcttggaatagatatttttaaacaaggatgtgggtctacaattattttagggtgagta
gatagggattggaatatctgcaatataggggtcttttgctacttggcccatttcttccat
gggtgaagacatgcttaagtgtatttaaaagacagccttttaggattaacgtgatttact
aatatggcgggattctgatggagaatggtaacatcaatattctttgccttgatagttatg
ctatttatatgagaggctggaagttttgtatctttgtgtgtcttagtaccagaggactcc
agagtctgtgtgagttatcagcagttcatttagatatactttgaaaatgattctttgaaa
agtcttgtgggacgacgtaaagaatgggttggcaacctttctgtgtgggtcagcatactg
tgtcacaactttcagctgtttctgccatgtgaaaacagctgtagataatatggaagtgtg
ttataactgttccagtcagatctcatgcataaaaacaagaagtagatgggatttggcctg
taggcggaggtttatagctccctggcagaggacagctaaatctaccagcatctactagtg
taagctagttatactagaaagccagattggccatttcaggtggattttgttttctagatg
agatgtgcttctctggactgccattgtaatgatgatttgcctttatagtaatgagaatta
tggatcaagaagcagagacttgatgttctgtaaatagatctgaaaaatagagaccttcat
cttaccagaaggtgccataattgatgtcttagttacttttctatttctgtcacaaatatg
tgaccaaggcaacttactgaaaaaaaaaagcacttaattttgcttacagtttcagagggt
gagcccttggtcatcattgtgggtgagcaggcataacactggagaagtagctgagagctc
acatctgacccacaagcaggagagactgggtctggtgtgggcttttgaaactgaaagctt
accccagtgacacacctccttgccttttcaaagcagttcaccaactggagaccattcaaa
tatgtgagccagtgggggccattctcattcaaaccatcacattggtatgtagggatacac
ttgttttgtattatggctaaaagaatctgttaaggaagaagttaactttctaccccactg
ctacagggataggagccccgagaagctcgaggcctgctaatcaagcattctcccactgag
ctgcatctctctatggtataagcttacttttgaaaggttctcaaaacaaagtataaagca
tttgcttttaatgtgaaagtaaaaaggcatttaagttgttttgaatcagacaaagatgtt
aagtcagactcaaggaaagttaaggtttaatgcttctacctcttaaaatcataatctcat
actaaatcagtgtgaagaattcagtacagaattttcattttgttgttaaatcttaaatta
cctaaaccacttgaatgtatactttaaacaacattgctatctttaagactaatatagaca
aataaatgtactacctagaaagaactagctattcacttagtgaaatgtttaatgttggct
taatgttaaaactttcgtttcatatgtcaacagaaacctgtattagatgctagtatattt
accctggttttacaaactttgtttcatggtactgcaattttttttttttttttttgtata
atacagtgcaatagtgatccctttttagttagtttcagaactgtattaactatagtgatc
cctctgtataggacccctgcagataccaaaatctgaggatgtttaagtctcttatgtaaa
ataaagaaatatttaaaggaattagtgagacagcttgacatcatgtctgagaacctgagt
ttcatccctcagacatccctaccatggacatgatggaaggagataacttactcttgaaag
ttgtcctctggccttcatatatgcagtgtatagtgtgccaaccctgccataaataatgta
ataaaagtatttacatataacatgcttatatatataaacatatatatgtatatacacaca
tgtatatctacatacctctttctgtgtactttaaaattcacctctaaattacttgtaata
aatataataaagtgtttaataattgttataatacaataaagtagcacattagcagttgtt
atgctgtgttgtggagggataacaacaacaacaacaacaaaaatctattaggttatactt
ttttttctggatatttttcaatttggttggttgagtctgtagacatagaactcacatgga
actgtttattctgtgtatatagtagatactttagtttctgttttgctctttgaataaaag
atgatataatctgacaatacttttctttttgggggtggggtgtaggctctttagtttgta
tgcatatgcaattctgtgagacagtctttcctgcccaggctgcttttcagtgtgagatac
tgctgcctcatcttctggagtgttgagattatagggatctataactatgcctggttttat
tttggtcttttaaaaatcttactttaaacattttgcttttaaaaataattttatcaaaag
atgggtgatgaagttaaatgacactggcactctcatgtctgcagggatttcaagtagaaa
tattttactttggcaacacttagtagaccaggaatggtgagctttttggtgtgtgttcat
taccattgcaaatagatgagtacatttgttttcaaatagtgaactcttgccatcacatat
ttctattttgagactcttctcatttagtataaaatcattctctggtaggcttgaaaatag
acaattagatttttttgcagttttctgatgttacctcactgatagtttctgtaatacctt
tcatgtatgcaaatggagtatgtttcctaagtgcatatgacatttatacatattctagta
attacatgtttacatataaatatagttaaaattattcctaagacattttgagattgtgaa
atccttgagaaacatttgcaattctcactgagccctgggacagtagaatgattaatttaa
aagaaaagttttataaaatgttcctttacgttttttaagtacactcaatttctctaatag
agtaagcttaaaaaagagaagatgagaagcaatatttgtgttgtatatcttccttgtgtg
taggtggaaatgctcaagagatgagctattattatttgttctttctcctaaagcatagct
gtgcttcagtcaggtaggtcctgtgtgctctgctgctaatttgtggcctctgccctttgc
ttccacctgaagaagaacaactactagaaatttgggtggctttggaaagagctagttctt
tttccctccctgtttgtgtatctgtgtactggtataatatgataaatactttttttctgt
gctgtaagacttctctgatgtttgtaaatcttaaaagctagtgtgtaatcaggctaacac
acttacactttaccttccctctgagaagtctctatgaacacactgtgtttaccttgggct
ttctcagcccagactagcctggaactcactaagtcatttgtataggcctcaaacttgaga
gccctttgtctcagcttcccaagtgctatcagtgctgggagtataggtctgtgccgccag
agtccacttgccaatactggtaacaccaatcagtatgattatttacaattggttgattct
tttaggatgtctgtttttttaggagtttaagaagagatagaacttggcatggtgttctag
gtctatgaatttgctttaccaaaagtattgagcaatttataactaactacaatcagtggt
tgtattcctggaaccagcaacaagttgctagctcatctctcatttacctacgaacacaca
atcacactttgggggcagaatactcaggtaattgagaacccatttcttagcttcctcacc
ttgaacaagctacttcctcagtacccactcactggggaaagcatgctctccacaaggttt
gctgggagaagctcatatatatgatttaccaatgttaatttactaaaggacatgcctttt
aatatgtaatgtaagtagaaggcccgagtcagccattattttgctcaacatttctgtttt
acggtttctattgctgtgaagagacaccatgaccaaggcaactcttataaaggaaaacat
ttaattggggctgccttacagttttaggggattagtctattatcctcatggtgggaaaca
tagtgtgcaggcagacatggtgctggagaaggagctgagagttccacatctggatccata
gaccacagaagtgagtcctacactaggcctagctttaacatttgagacctttaagcctgc
tcacgtacatagtgacacacttcctctaaaaaggccacacatcctaatagtaacactcat
tactgtgggccaaacaatcaaatacatgagtctatggggaccattcctattcagaccact
aactacaattcacctttaacaccacccccgagacagggtttctctgcgtagtcttgactg
tccaagaactagttctgtagaccaggctggcctcaagcctttgccaggattacaggtgtg
tgccatctctgcctggctgattctatccaacatttaaatgagcacttgagcttgtagtcc
tgtttcataagcatcaaaaatgaatatatgtttcattaacattttattaaaaagtcctaa
aaactagggaaatgacagaactatttatagacaaagatttaatcaaatatcactggactt
gtagttaactgccaaagacctcaagtggtgcatggaacagaaaggtttcctctttattct
ggaacaattagtgttttccttttccttcttctgctagtgatgatgatttgagttgtccag
gacaaatcctgggatgcgattcagttctcagttcactgataactttactgccaccactgc
cccttcaacactggaaatacaaagctttggaaaatcgtaaagctggatgcaagatttatc
tttctcagttattcagactaagattgaaataccttgatttagtgaacacattgttagttg
atacctaattttattttcataaaatgattgtgtgtgtgtaagtaatgtgccttaaataca
agatagctttttcttcaagtgtagaatagtcggttttaacagcatctacaaggaatagtt
gcttcttaatggtttaaagagaggatttatgttattttaaattctgtgtatagatctgaa
tgtctgtgcataggtgtgtgcacttatgcaggtgcctggaaaggccagaggtgtcagctc
ctctggagctagagtttctggggattgtgagccacccagtgtgggtgctgggaacaaact
ggggtcttcagaaagagcagtgtctgcttttaacatccaacccatctctgactcttgatt
tttttttttttttttaatttggaaggtttttctggtttgaggaccacttggttatgcttt
tacttacattttacttgacagaattatacatttaatttataaaagctcacaggcaggata
aaagatggaaacatttcttagcagtgatgatgtttatgctttatgctccattcttcatgc
tttattttctcctttgtcggcagctttcctttagattgtgatttaaatgcaaaatcaggt
tgcttatctgcacgtggatctcttgttagattgtcaatttgatgactatctaatttttaa
aaatttcaatttttaaacttttttctttttggtagcaattatagctaggtcatctctagt
tttctttccacttcagttattttcaaaaaggatgctgatttctataagaacgcagtcacc
tagccttctgtgtggtcagtagctctgagctgtgggtttgatgcaggagtgcagggcgtt
tctctacggatcattacacagtggtgttgttttcattaaggaagataagattttaattta
ctggattgagaaatttggagtaggaaacatttaaacttgttataaaattcattgtggggg
agggggaggggtggacggtacagggaagggacagacagcaggacagaagagggagggctg
aaaaataaactgggggagcctgtctccggaagttttgaggtcagtcttagtcagaggtca
caccctgttatttctttgggctgtggacctggggtgttaccggttgttatctgctatact
ttgaaatgttgattagaagattgatttttgttcttggcttttccacatctggattcatta
aaccgttgaatttctaatcctttttacataatcagtgtgtcctcgggtggtagaatttcc
actatacctgataagagaaggtttggacaaaaggtctgatctaccctacatttctgattt
ctgtaagagggatgctaataattgtatataaggagctaagaggcaagctttgagctgctt
gctcaagttcattgtgcttcctgtacaactgtaatgataataatagtacctacagcactt
atggtattaggaagactggcattgcgcatgcgagcctttagcagtatctggctcagtgcc
cactaagtgctagtcactgtagtaatacagagtgcagtgttacgttattgttgtatttgt
gaagttgctctgttttcaaaaactgatgtcatttgtgatctttgcttttgaatatgttcc
tattttgaatactgtctttattcagcagaagatatttactgaatacctactgtgtcaggc
attgatctaagtgaacaaagcataaaaatacctgattgctgaaacttatctctggttgga
ggaggcaggacacacacaataaagcaaaacagaaattgggaggcagaggcagttgaattt
ctgagtttcaggccagcctggtctacagagtgtgttccaggacagtcagggctatacaga
gaaaccctgtctcaaaaaaaaaaaccaaaaaaaaccaaaaaaaaaaaaaaaaaaaaaaga
aagaaagaaagaaagaaattgggctggtagattttgtgtattggaaaccaccgttaatga
aacctgctagtctttcccctgcttgtgttgctttgctttatttaggaagcacctgctggc
ctgtccccagctgtctgtcttccgtggaaggtagcctcaaggctttcagcattgccttgg
cttactgcttacggtggctagctcagcctttgtcttccagtcctccacctctgtttgagt
cttggctttgcttctccccagtgtcccacctttggcctttcaactctgtcacactcaaga
gacagttcacagaaaacctctttttaaatgtatttcatgtttaagcagcattttcccatt
ttgtgtttcctcccttttcattttctttagctaggatagttagcttgcttgccagtctca
tttaagaaatcctatttttagccgggcggtggtggcgcacgactttaatcccagcactcg
ggaggcagaggcaggcggatttctgagttcgaggccagcctggtctgcagagtgagttcc
aggacagccagggctacacagagaaaccctgtctcgaaaaaccaaaagaaaaaaaaaatc
ctgttttttttttttttttttttgtatcgtcaattcagttgtgaatcttttaaggttaca
gaccttttccattccgagctttcttaacttaggaagaaaggataatacttgctccaagga
gtgttgtgagttttctgagagttatgtagccactcgggttgctgcagctctggccttagt
gctcaagagcccttgtgtctgtcatcccgcacctagactaggagagcttacagtttcact
tccattttcttaatgggtcacagttttaggtttcttttttttttttttttaaatgataca
atgttacttttcaaaaatcacatgaaaatatgatgatgtaacaatgattataaagcagga
attcaaggcccatcatgtgggtgctcacactgattgtaagacctttggcaagttcctttc
tctctcttcagtgtatccatatgttagggaaactaatacctaccttacggatgcttttag
gaaaaatgagatcacaacactatctggatgtagtaactaatctctcacagtggaggtgtt
tctgatggtctcagctatgtggagagaagacaggaggagcagaggggacagtgtgggaga
aggaggagaggaatgaattaactcactcactgctgggatctaatgactatagaaactgct
ataaatgatatatagggcactattcaaagcaaaaaattatctggtttaaaataccattaa
tgccagtatcgggaagcctgctttgtaattaagtccccactctttcatttaggtcagttt
tttttttaatatattttatcagttagattctgtttaagaattgattgttgataagcataa
acatggataggacttatttaatattcattctaagaaagacttaatagggaagtcaataga
taaaactttttaaaaggctttttctgaacctggggaaagtttgccatattcctgaaggtt
ttattaattttttatttgttaatctgctcatgtaaaagtctgttgagaactgagggcttg
tgagagaggagtgccagaactcgtgtgttgtaaagcgagcaacgtggacactgaaaggaa
aagcacacatccaggttacggaagtgacctgatggggaacttgccttgtgtgttcttttt
ctctccttgtcttgttttattacttgacaagcaaaggactttactgtctgtcttctcttg
ttatctttttctccttgtttaaagttaaatcttgctattccctgtggagcctttcctttc
tttgctcttgtcctttctgtgcattttcacctcaggggactgggctggtagcctttctgg
tgcagatcctatctacatgcagacttcctgtatcatcctctaattcctgggagactatca
gcctaaagtctaaagctttaggagttgggaggagcagacgtttctctcttggtgtcgcct
gtgtgctaagaaagtagggacataaatttcagtctttgtcattcacttgaattttatcct
ttgtatgcaaaagacaggctgtaccttcaggtgtctaaaacatatgcatactttgtgtgt
gtgtgtatgtgtgcgcatatatgtgtgtatatagttgtctctcagtattagttctagaat
ctcccaaggctgctagactctgcagcactgcttctaaccaactgcatcactttgaatacc
ttgtctctaggttacttctaactaatactgtgtgaaggtgatggtgatgtaaacaattta
aaaccctatgtttaggacaaaaaaaaaatgtgtgtattcagttgcacaaagcaacttttc
tttgtgtgtgtctactcttgcatatgtgtgggtagaggccagaggttgacatctgctttg
tcttacatgtttttcttattttttgagacaagtctgtcactgagtctggagctgatccat
tgggataggctgactgccaggatgctctagagacccgcctgcctctggcttcctactgtg
gttgggcttcatgactagtgtttaacattcctcctaggcattggaactgtctccatgctt
gcatagcagactgttgtattgattgatccatctcttccagtacaacatcactttatagac
tattgtttgtttgttggtttggtttttggtgttttttttttttttttttttgagacaggt
cttctctatgtaacctttgctgtcctggaacttgctctgtagtccaggctgacctcaaag
gctaactttttatttctatctttagtgtttcttcagttaaactgaggatgttgaatctgt
ggatttggagcccatagatgttaaaggccagctatactttcatgtatatttgcttctgta
ccctactattctctgcctggaggagtctttcttttcttagatcttacttaaaaagtcatc
ctaggaaacgtgccctgattaagtagagtgagttattttttacttacagtgtctttagcg
tttacagaatcagatgtgagtctgtttttctctagtctctttgagcataggggtcatgtc
ttatttactttgtttagtgaatttccctcccccagataccttaacattactgctacatag
gatgtgtatggcttgtgaacaaacatcagaagagaacacacagaaaatcttttcaataat
ctctaaactggaagagattagagattattttgtcttgttatctgtctattcaggtgctta
gtagtgccttctgtagaggtattctgaaaatttttattgggtcaattagaagcaagtttc
atatgatatattttttttatttgcacctatgtgtatgtctctgtgaatgccacatgtgtg
ttagtgcctgtgaatgccagaagagggcatccagttctgtggggatggagttatagacgg
ttgtggcccacccaacatgggtattaggaactgaacttggggatctctgcaagaggtgcc
agcactcttaaccagtaagcctgagagggaaaacttcatagtagatattttctgtgtgac
tttccttcttcctcatttctaggaagcatgtagtactaagacagctttgtacccctggga
gaatgtgccttatgctggcttttcttgatttagtaggaagattttgagacaacacaatca
gtttaaacttttctttaaacttgtctgcctttctggggctggagtgatggctcagtcgtt
aagagtattggctgctattctggaggacctggctcaattcccagcacccacatggcagtt
cacaactgtaattccagttccaggagagccaacactcccacacaaacatacatataggca
aaacactgatccacataaaacaagtaaataaataaatctatcttaaacaaacaaacaaac
atctgccttttgtctaagagattagatttctaattatctttcagatattgcctgttcaga
aacttttaccattgccaaatttttcatagcctgcatgctgagctgtgtaatccccgatgg
gataatgacccatttaagaaactgatctaaaatgggctgaaatgatgctcttgattgatt
tcttttttattcatggtccttgtctggtgttgtgtttgttgaaaaaacaagaatgttgct
tattagccttacattatataagagctgttattaaataataataatactaaatgaaacttg
aaggcaaaccaagtgaatatgttttttcaaatgttggccatagctgaggtattgaggctt
gaagtaaggaagtcgccactgcatctctttccttttgatgatggattttagccttagact
tgcccaagtaatttacaggtagttagctggaagggagaattatgtgaatctaggggatag
gcatatagatttgatcatctaataaatgaataatttgatatttgtcaccacagtcatgat
tagcacttagtgagtgcttcctgtgtgtcagtcactgtgctaaggactcccccacacagt
gtttacagtttacttcttactttatctcatgctgtaggaagaggaaacattgagagcatt
gaacacaaggctaggaatttgaactcccagaagctctgtaagctgcactacacaggagca
aacattgtaggatttcactcttagaagcagattacaggctcagaggaaagcagttcccaa
tcattgtaaaatttaagtctgtatttgaatgactttccatgagccttagttttcagtgtc
acttttgccaggttaccaagacattttgcttcacttgcaggagtttattaaggtaaaata
tatttaaaacctgaagcagggatgcagtgggtgcagttgagtggtagtgtgcttgcctag
cacgtaccaggcacctagcactgccaggacgggcgtagagtgaagggacttgtcaatttt
atattcaattcataattttaaaaatattaattaactaattaggacaaggtctcaggcagt
atgagctgaccttgaactcctgatccttctacctctagttccccactgctgggattacag
gcatgcactggtgttttcagaggaggagatgaaactttgaaatatcatctatgaattgaa
aattatggttggatatatatgtgaagcatagcagaaatttgagaattaatacttgttaat
ctcttgaatatgttgtttaagttagtgtggttctgtggaatatttccctaatgttacttc
tttttggtcaattaaagtatgtcactttttctattctatactttgtttagcatgagtccc
tactcagtatattattgacaatgcatttaagaagtacttctgatatctcccctagtatgc
ctttgtatgttaactgggaggcttaaaagtttcacattttaattctttttccttacaaga
gctaagcctggagattgttttttaaaaaatattttgtttatttattttagtggtggaact
caaaaccaggccctgtgcatgctaggtgggcagtccaccactttttagtaaaagacttca
ccgatttgagaactttaaattctgtgcttttataaaatatgtatcagaggttacatgcac
gagtagagtatctagtgatttattaaagattaaaaaatatcaaaatggggtggctggaga
gatggctccacagatgagcttgtatgtatggcttttcctagattcagcactcctaccccc
agctcataacctatatctctctgctggggcaccagccctcatgtgtacatgcccctacac
cacattgaaaaataaaataaatacgtggggaagaaggtctgaattggtcctgagattctc
aattggtacttgcttccaggtcttgcaacgttgtttaatccctagaactgacatggtgga
gagaactgactcccccaggattgtcctctgacctccacatgcatattgtggcatatgtac
agtagcatacaatcatatatacatacccacagcaaaaaagtaacatcccctttaaaaatg
cataaatggtgctgagttcagctatggaagctagttgagtgattgaggcagagagagtca
gagtcttaagttcttcacgtccttggacattattgacctttctaccacaggtttctttct
taaagtggctgttagtattggtattgtagaaatccattcctagaaacattgctgaatgtt
taatgccagatactgcagtgtttatccacctctcacatactcctatttcatagtgcaagc
taggtgtgaagatggtgtgtgtttacattttcaccttgacttcggagttgacctaggatc
atgtgcaggagacatcaatggctaccctttactgcctgcactgtttgccagtttatttct
tatgtatttttaatagaacccagcccagcccatgcatagaagtgggggtctcttttcggg
ggcagttagttatgtattctctaagtttggcagcacatgcataattcttccttttactga
ttttcactatttatacattgatttatttctttgtatgtgtgcatgcgcgtgcatgtgtgc
atgtgtgttggtgcctgtgggtgtatgtaggcctgtgggcacatgcccacaatgtaggta
aggaagtcagaggcagcttgtgggaggcagttcctttctttcaccaggtgagttccaggg
gtcaaactcatgttgtcagccttgggaataatcaccatcttgctggccctgtttttccta
ttacttagtttcagactttatatacttgtataaaaatatctatctatctatctatctgtc
tattataatagaataatatataaataacaagatatttcctaaaatttcttgaattggcca
tgctggatctcaagcagagttttttttttttttaaccattcactgattctcgtaggcgtg
gatgagaactgaggctgcctctttcttccattctctttcgttccttttccttctctttat
aacacccccccccaattctttcatttccccagttgcttaggaagacagacagatgcacac
taaatttataatctagtttgtgcacacagttgatggaggaaacacagccttccaagttac
atccttttttaaactgaagttttaagtatgtatcttaactgactggggagattgaattgt
cacttatgctaatgtgatatattggaatttgggttagtgatcaagattggcatcattaaa
gagaaacaagtcatagtttttcacatctgagaagtcacctgcttttcatggctgcataaa
cagtgattacaaactcagcacctgagagtctgcctaggagtctggatctgcttccgctgg
gtccttcctcagggaacacccaggctaactgaagttcatctgcctgttgcctctcctcct
aatgctctgcttgtcttagaggcttactggttggcagaattagggttgtagggctgatgt
ctctgttttcttgcaagccacaagtcagggatccttttgagttcctcagaggcctgtctg
tgttcttgccacatggccctcccaccagtatagatctgaatcaaaggttagtgatagaaa
catctgtatatgtgcactgggatggcaaatggcactgtcacatgtgcttgtttgctgctg
ttccctgagtactttcagtcaactttactgtggcataatttatatgaaatgagctcatct
atttaaaatgtatgatttgaagagatttgagatatgtatatgtcatgaaaacactgccac
aagggaagacatttcatcattctttaaagatggttccttgtccagtctggtagtccctcc
tgaaataacttcatcttctgacactcattgctgctttctctagcctagttttactttggt
attttaaatagcatgatgaaaaaagaggagatggtgaaaggttattcctccatttttttg
tttttgtttttgttttttttttaaatactattttatcttactttacgcctatgggtgttt
gtttgtgtggaactctgtgcatcataggcatgattggtgccagaagagggtgtcacatcc
cctggactgttacagtagttgtgagccaccatgtgggtgctgggagttaaaccagggtct
tctggaagagtgaccagtgcttttgactgctgaaccatctctccaggtccgatacatatt
actcttaacttacgctttcctcagattccttcttggcaaaataatcctttatcagataat
gctttatggttttgggatgtggtaaaatgattcttgtattttctcaagatagttgcagta
tgctcttgttctctgttagatctccaaagctagatagatttaaataggtagcttcctcat
ctactgttccagttactgtctcttaaggcccaaaccaggtaatttcccattagaaattct
agtagtagtgaatcttttcactggttactggaaacccaacccttctagagaagtcagagt
atactcacctcagactttctgttgttacagttcatgtcacagtgtgaatttagaagacat
gaacagtttcccttgtagtcacttactgtcaccccctccccctgctccttgtataatgca
gtgttgtgcagtgcaggaaggttttaggaagccttgcttcttgcagcagaagaaaaagag
agctcagaggagactcagtgagaccctctaaaagctgtcaactgtcaacgagacagtgtt
gatcaatgctgtatacctgaatgcaaagtttctactaatggtattgaaacagatagtagt
aattttagtgtatttttaaaatgtgtttactatatatttgtataatatatccaaatatta
ttacttctgcatttaagctaagaaaacttattgaggtagtgttattttaatattaatctt
tgagttcctgtgcatattttatcctttacacagtagtgtcacttccttgcttccctccct
tccttctctttttcctccctctttggcatgtagcttagattggccttgagtcccctgggt
aacaaaagccggtttcagacgcttcattcttcatccttttgccctgttggggccgtagac
agttgcaccgtcccaggggcccagaggtgagtctggtcaggttgtaatagcaggtgctcg
ctatctactggcccctccactagacagtgcttatgctccagataagcaactctctctttt
atgtaacattacatgactccctaagaaaccacctttgagtaaatgtttgattatcttgta
ggttagggtggtagctcagaaggtttctgtggttaattttgtgaaggtaaatatactgta
atacatgtttattatacagatgatgacattattgaagaggtggtggggtctgggaagaaa
cttgcttcctcagtcaagagttaactagtggtaaacttgggctgagttttcttcttttaa
gtgtttcactttaaatcaaagagaaatgaattcctttttgatcttactctacatattaag
acatttattgttagcatctgttttactgctagatctttaaagttttgtatttttttctca
gggttgtactcactacaggtctgcttgtggtattctcacaatagtctgaatgtttcctga
atagaattagaaatctgttttaatttactaaaattaaaacagtcttatagctcaaggtgg
cctggaatttactatgtaggtgaagatggtcttcgactccttatccttgtgtctttaagg
agtgctgccctaactgcttaagggaagctatctcctgtctcacccatacttttcaggcac
agaaaaagaggtaaagtagaataaatgggaaaatggaagccaagagttctcttttacttt
gtttatagaatcagaagaccatgtgagggtctgttgtgccctcccaggtgttcctgtcct
gtctctcagcgtaaccttgtctgtcctggaactacagtgtatgccaggctactgtggaac
tcagagatctacttgcctccctcctgagtgcagggattaaaggtgtgcaccaccattcgt
agctttacctggcatagtgcgtctgtttaaatgaaggaaagaggacttgttttttcactg
ctgtgacagaatactcaagaaaaccaaggaagacaagatttattttggctcatcatttaa
gaatggatacaagtttgtcatggtgtagaaggcatggcagcaggaacttggggtgtctgg
ttgtgccatgtaacctagagggctttgcattttattgagcagggggtcctgcatatctgg
gcctgtctggctgcttcatagggtagcaggcacactgttgctgactactgccacctttgc
agagctgtagaagggttgtgttaactcactgagtgtgtcctagtttccttctacctcttg
cactgttaggatgatgttccacattgtaagtaatttctgcctcagaaagcagctccagca
gtcactaaaggaggactcccagttgctatgagaaccttaggcaacgactatgtaacagaa
tgtctcaccctaaaaaactaaaaaagaagatttattacttatttgtgtgcctgtgaatgc
caggagagggtatccagtctcctggaactggaatggttgtgagccacctgatgtgagtgt
tgggatttgaactcaggtcctgtggaagagcacaagggctctcaacagcggagctatctt
ctcagcctcagcagttcttttgatgccacttttccttctgctacccaggaagcagtgatt
gctcaagttcacctcaaactgtgtaaaacgcattacctgtttattgtctctgtgcgtgtg
ttttctctgggccagagtctaactccgaaccactcacgagtcacatttatttgctgctgt
attagcaacttcctattgctgtgataaaacaccacagcaactgactgaaaagtgtttgtt
tgggctcagagttccagagcatagggtttccttcacagtcatggtggggcagcatggcag
caggcaggcgtggctgctgcagttggagcttttataacttgggccacaagcatgagacag
aaagtggcctgaggatggtgtgagttttttaagccccagaatcccgcccccagagacata
acttcttttaacaaagccacaactcacttccccaaacagtaccagcaactgggaccaggt
atctaaaaagctgagaatatgggggacatttcattcgaatcaccagtgtccttaattttc
ttcagtctgaaacatttctaaaattcttgccttttattattactttggaaaagtactggt
tgtgttgaatacctccagtttttgtttaatgttttggtgacaattgttgggtaagagtac
tacagaaatgctgttgtgactcttactggatggcacacagtgtcagtatgcccattcatc
tggtgagaccagcactggtaagctgatgttaggctttgtaccagttgcatgctaaccttg
caattaagtgtcctatggggaaggtatcttgtgattatgttaattaccttattttcatca
tgcttttagtttctaatttcaaattctggtgataatttgtatctgaatgaatattattac
tttggtgtttggccagtgttattttctgtttcttttctttcttctgtaaggaagagctat
ctcctctgaggtatgtactttaagtgtatcaaaatagactcatgaatattttctctatcc
tgtagactcagattttattctctggatttcatctatttctttctcttcctttggtgcttt
ggtcatctgaggtttggtcctcaggactctctcaatctttcatcttgtctcaatcacttt
ttgaggacagattatagcagcagcatcagttgtcccaggcttatcttgtccctttttttc
ttccctagccttcaatttggccatctcaccaaggagttatttgtttatttttactgtggg
ttgagttttttccatttttttctccattagcatctttaaaaacgtttgtgttagaatgac
tttggatggcacagacatatgaccctgctgtaggaatactagtgcatagccagtggttgt
cttgtgatagactttgtctcccagcaggttcactcatttagtcatggatgagttttacac
cttaacattgctcacatgttactccatggaaacaactgatatttttctttggttttgttc
tgtgtttggtaataacaatttaaaaagctcttctaaaagtttccttctaatatcacatag
tatagaaatgtttatagctctttatttatctttatttatttatttatttatttatgatgt
tatgatagtttcagtttttcaataaagtttaaactgcttctacgttcttctagttcactt
ccatgtcagagatggcttactctctagaatacatttgtctgtctgtctgtctgtctgtct
gtctgtctgcttgcctgcctgcctttgtgtgtggtttgggatggtgtgagctatatttat
atgatgcatgcacatgagtatgcaggtgtatatgcctgtgcacgggtatgtggaagttgg
aggaagtctttgagtgcctccctttttggctctttgcggtattatgttgacccaaggtct
ctccgtaagccaaaagtttggcattccagctaggctgactggctagtaagctcctgcttc
ctccttccagtggtctagctgaggtcctcatgtttgcacagcaagtgttctttctgtgag
ccggctcccaagtcctgcagtgcattttacatcaggcttcacagtgcctacagtagattt
catttttcgtcctgctgctttggtttttaaagcaatgtttgttctcaatttatttcctat
ctctgttgtaaactgcagcttgaaatttgcaacgcctggaaagttggaaacaaggaaaac
atcatggagcatggtttataagctacagcttcccagacattgtgcttttagctaagcaag
gcattaactagtgcccccttgtacttgggctgtgtcagtttgcttcacaaaattcatcca
tacttcccattaatttatcccagttttatttctaagtattttcccatggtcagccatggc
agagtcaacctcaactccgaatgggcacaaacacacagatgttcctggctgcagccagag
agtagtgtacttgctcacttctccaggcgttcttgatgtgttctgaagctggaggagtac
tgatgcatttcctagtgactgcttcagaccgcactgggaacaggcgtgccagctcatgct
ctctgtctgttgaaagcagctcttaagtttctgcatcagtctgctgtttttcagctgttt
cttgtcattttttttgcagctgatttgcttaataattctaattagcttttgtttatgggt
ttatgttaaaaagtataaattttgattgttgattgtgttatggaaattaaatttcatcat
tagtgtctatacttgggtagctagctcacactaaacttgacttttggatgcttcagttca
gtgtgttcatatgcagtgagatatgagaaagtaaacactttttttgttgttgttgaatat
tgaaacacattttgtttagtttagagttaatggtgggttaatttctacgaaattctttta
ctattcatccagtgaggagagagcacaagatctttaaatgacttgtattttgttttcatt
cccacagactgggtaggatcactattgtattgtattggcacatacacgctggacagctgg
tgaactgtatccccaatctttcataggctgtctcagaaaactgagcacgagcattttcaa
tgtgtgtcatacagggactaaagctacaagagaaatattgctttcttcttagatatttct
agcatagaacacttggtttgataaacctaatttttagtttgctattttttgacatatatg
aaaaattagtaaggtccatgtcattatcagtagttattacttggttattaccattttggt
ttttttgacacagggtctgtgtgtagctggtgatggcatccaactcacaatcctgcttta
gcttgttgagtgctggaatcacaatcctgtgtcaccatgaagggcttattttaatttttt
aaaaagcagcatcttgacagtatttcttttatttggaagttctttcagaacttgtctgaa
cttgtctgaagttgtgattggcagtgcccttgtcatttgactcatttaaaactgaaggaa
gtaatttttcaaaatttgagtttattgatatttttacaaaacatcttcagactagccgag
agtggccttgactttgggccctcttgccaccacttccctagcgcagggattgtaggcttg
ctccagcatacctgcttatgcgatgctggatcaagcccaggtccctgtgtatgctcagca
agcattctaccaattgagtggcatctgccagctgagccgtattgccacttgagctgcttc
cttttgtatccactccttagaagagtataattttaaattgtgtataaaagataataaaat
aattcagatagaaatcaaaaggcattttaatttagtaaccatctaagatttatgtaattt
ttttttccattttttattaggtatttagctcatttacatttccaatgctatactaaaagt
cccccatacccacccacccccactcccctacccacccactccccctttttggccctggcg
ttcccctgtactggggcatacaaagtttacgtgtccaatgggcctctctttccagtgatg
gccgattaggccatcttttgttacatatgcagctagagtcaagagatccggggtactggt
tagttcataatgttgttccacctatagggttgcagatccctttagctccttgggttcttt
ttctagctcctccattgggagccctgtgatccatccattagctgactgtgagcatccact
tctgtgtagatttatgtaatttatataggcatcctcacaacctgtattgtaaaaaatttt
cagtaaatacattaaactttatattgaaagttgaaaaacattcacttcaactgaaatagt
tagtggatactgacaagaccagtactatttttatgtataattttgaaaaggcttatgtca
taggaatattaagtaccagggaactttttaatcaagagccccgggaagagctgctcagta
ttaaaattttctttgtagcatgcttcactgccacgaaggattaaaagaaatgataattca
aacattgttcatgggtaaaaatctatgtaataagctggcaggatggcaggaaaggtgatt
gccaccaagcctcatgacctgagtttgagtctgatgacccagccttgtccctgtcactca
tctgatagagaactcacttagatgagttttcctctcacctttatacacgtgctgtggttt
gtgtttgtgtgtgcatgtacacacagacagacagacagacacacacatacattttagaaa
agacgataaatcaggaacaatctcatttaatagctcttttcacaatatatatgtatatgt
ataaaggtctcaagtttagaacaaatatttaagttacacattaaattctattgtgggaag
attgttaatagatctcaaatgtttaatattttgatttttgagtagattatcatctgactg
tttctacccaccagctcttaataatgacacggagactatttatttatttatttatttatt
tatttatttatgaaagcttaggccttagcttgttactttctaactagctcatataattta
attaacctgtttattctaatctacatttgccatgtggctccttacctctcactaagtctg
atttcctcagtgtctggctagtgactccctcacttctcttttcttcccatagctactatc
tctgcctggaagttcccctttcctctcctgtctcagctataggctgtcagctctttatta
aaccaattggaaggtggcggagaagaatgtttacaaaatactcaggcaggtgatgatcga
ataaaaataccaatatgagaattcagtacatttgaataatacaaagacaacctttacaca
atgcacagaaagattatcccaatagtagatacactttttttttttttttttaaagtttgc
gtcttctgtaatgtacttttaaagctagaggtagatgtgtctggagtagttaccacacca
gactttctttctcttctttgattaagtccagcagcattaagttcttcatgtctgatgacc
acaggaataaggagtatttctgctggcatttatcccgctgactgttaggatttgcacaat
acatggtgatgcccagagaattgctttaaagtcagaggaataagcccatgtaaaaaagga
ggagacacttgagcaatcctgtaagcttttcttttgagagaattcgtccagtagtttgtt
tgtgtaatgaattctgtgacaataattttaggaccactggtcctgaagagagttttgtga
ttaagttgaagaaacaccatatactgtaactatagcctattaagattcatagtgcacatt
aatatgctaagggttctgagccatctcttgaaaaagaagtttaccaaagcttgcttatcc
tgtatttcttaagtgtcttggacaatggaattctttcattcaagcttctctgtcctgtga
taaatgctgccctctagaggcataaattcaccggctcttcaatttgagcaccttaattta
atacttagtgagtagtaagtggctcttaacaccaagtgcacaagagtggaatagcctagt
agaaaaagatggtctgatttaattttaagaaaatgtatgctctaaattggctttattatg
tttactacaatcatctttgtaatcagttctctctttggtggcacttgggatgggatctag
ggctttgcatgtgctctgtggaccttctactattcactgtgctggaataacattctttta
atagaagaaattgttaccgtttaatttttattacattttaattttgggaacacatacata
gagttcagagaataacttccaagaatcagttcttacctcctcctggggatcaaattcagg
taatcaggcttttcatcaagcaccttgacctgctgaactgtcttgtggctcaaatatagg
ggattgttgacacataagtcttcatgaacctactatgcagttcagattggccttaaactc
ctagtgaacctcctgcctcatcccaccacacccatctttgtctactttattgaagacctg
atacaatcttgcttaagagagtgagcagttgttcccacagtccttatgtttccagacctc
taactggagccaagattatgtcctacaacaccctcatctacttagattatgtcctacaac
accctcgtctacttagattatgtcctacaacaccctcatctacttagattatgtcctaca
acaccctcatctacttagattatgtcctacaacccctcatctacttagattatgtcctac
aacccctcatctacttagattatgtcctacaacccctcatctacttagattatgtcctac
aacaccctcatctacttagattatatcctacaacaccctcatctacttagattatgtcct
acaacccctcatctacttagattatgtcctacaacaccctcgtctacttagattatatcc
tacaaccccctcatctacttagattatgtcctacaacccctcatctacttagattatatc
ctacaaccccctcatctacttagattatgtcctacaacccctcatctacttagattatgt
cctacaaccccctcatctacttagattatatcctacaaccccctcatctacttagattat
gtcctacaaccctctcatctacttagattatatcctacaaccccctcatctacttagatt
atgtcctacaaccctctcatctacttagattatatcctacaacaccctcatctacttaga
ttatgtcctacaacccctcatctacttagattatgtcctacaaccccctcatctacttag
attatgtcctacaaccccctcatctacttagattatgtcctacaacccctcatctactta
gattatgtcctacaacccctcatctacttagattatgtcctacaacccctcatctactta
gattatgtcctacaacccccttatctacttagattatgtcctacaaccccctcatctact
tagattatgtcctacaaccccctcatctacttagattatgtcctacaaccctctcatcta
cttagattatatcctacaaccccctcatctacttagattatgtcctacaacccccttatc
tacttagattatgtcctacaacccctcatctacttagattatgtcctacaaccccctcat
ctacttagattatgtcctacaaccccctcatctacttagattgggaagaagaggattgtg
tagtgtgttttaagacttgcaaaaactttatacgtatgtatagaacttgtttcaaaacat
gggaggggaggaaaaaaagaggggtgaaaccacgtacgaatagacaacatgcttttgtat
ttttgctgaatgagttaatgtgatttacttacagagattctagtttcagtgttttagttc
aaatagttctaattatttgcccttttgattttggtattgctgtgcactaactgaaactca
tggtacagtttaatggaagtgaaaaagttaaaacaaaacaggattgatgaagtccattta
tcatgttgaccattcagctatcttcctgactctccagaaaattccacttatgatcttgaa
catactgcatttacctaggtgggaataaaaacctttgtggcactgtgactgctgctgtgg
gtgtcttttccctctttggaggaagggctgataggattctagatgctctcagttccgtaa
gaaagataggcatcatttcataaggaaacatgctaagtgtgatggtcaaagaaacactgc
ataaattctccttggctatgactgtaaaatggtagaattcctagatctcaaataggaggg
cagcccattagaatctgttaagttctgtcccatgccccaccctcctaatgctggggattg
aacctaggtctttgagcacgatagacaagtagccctgctacttttttagattgtatttca
aggtagtatctaagttacacagggtttccttgagtttgtgacccttttcttggcctctgg
gtatctgctagagggatgactaagttccttcttatatgcttatgtgctaatacattttcc
ctcattcttttcattcctactccccttttgtaatttagtctgtaggttatagaaaagcca
ggcaacggtcatcacctagaatttctgctattggagctgctgtagcagctggtgggactg
gttttttaacgtttttgtgaggagcagaagatttatacaagtcttagagtttgtcatctt
gagaattttgtttagagtttattgttaaaaataattgtgtgtgttgggtcatgtttttga
ttgtttatttcagattgcttattggtattgggatactatcatactgggctataattcaca
gaatcctttgcaataagatacaggcatgtggtctaggcactgtgcattacatggatgcat
aagatacaaatcccaaagagaagtgcaagctaacagctcttgattggaatggctgcttca
tgtagtttgaggcttgagaccctgagcttgtggggcagcagtgcggaggggccagtgacc
ttgtcctgctccctgggcttttgtggtactagagtgctgttatggggatcatttgatgac
tttgctgtttttctgctgtacagcctgtgagtggatgctttctctgagagttacttacat
gttttaacaagttcttttttgcaacttagatcctggttttatagatttttgtaaatattt
tatttttctgagacttaaaaattgccaagcacatctttcacagttttccttccccagagg
tttacattttgacaccatttttcttgcttggtgtgtgttttgcacctttggattgttttg
atgtttgtccttcacctcctcagcttcagttccactgtagttaggaaacctttcctccag
ctggcactttttttccctgaatgccagccgaagttgtctaggtgtgaagtgttgcttttt
ctttctgtatgcatcttagtggcacactagtgataagctcccagtgagctgctaacaggc
ttttctgtctcttgcattaccaggcagaatgcactgtttagattcaaggtcacctgaaaa
atcacagtgaagccaggccgagtgagctgagataacaagctagctacatttgattcacct
tagcaccctgtccaggatgtccaatttccttcttccttacagagactcagatctgctctc
attaggaggtcatggcctcgccatctgaattacttactgtctttgtaatggtttgtgatt
tttgccttgacaaaaacctgtttgtctccaagttcctttcattgtaacttttggatttgg
tattcatcctcagcaagcttactattttaaataccattataactctataaataatgaagt
aaatcagacacactgtagaaatccagaaggtactaaacatacctcttgtgcccaagtcag
ggttaaggagccattaacaacctttgtcctcctttgcatccctcatctgatgctctgaac
tgatgatcctcctccagtctcttcctgacctgtaggggctcttcttcctttcaagttctt
tccttctcagctcgtcttcctttgttactaggtacccggtggcctaaggtcctttggcca
ttccactcttgtttttctatgtctgagatgttcataaaatgacctgaacagttttacatt
tcattgctttatagatgcattccaattgacataaaaaacttagttcatttcttactgcct
ctgttacctttgcatgttgagtaagagtcagttttgctgagagataactaggcttttttg
tctttggcaagtggtgcgtaatgctcgcacatggtatcgtaatcaagtgtgtgagttgat
ttgcgataggcaccagaaagtaggggctcagaggatctaggttcaggtattgctttgggg
tccaaaagttacagcttgatacctcccaccctgtttcccttaatttttatctagtggcct
ccagttggcctgtgtgagtttgatgtaaaatttatttattactttaatttctagtgaagt
tgagcatctttcatcactctgatatcctgttctttgaatcttttcttcgtatgctctaca
tttactttttgtattttccctttatattgatttctggcactttttatagtttgtgcatta
gtgttttgagaactaagcattacaattacttttctaatctttgttacactcagttctgtt
agtgttttagtttgttgcttgtatctatagttgatgttgagtgtcagcctgatagattga
gccaagcctaggacctcagtaacaagcacttctgggtgtttctttccagagtggattaac
tgagggcagagctgccctgactgtggactgtaccattcagagactgagaggcagataaag
tgaatgtgggaacgaaaatcaggtgtacacagctgtaatcccagtactccggaggtcagg
agttaaaggtgcttttggcctacacagaagagccctgttttcaaattttggggattgaga
atgtactccagtggtagagtgttagcatagcatgtgcagccttgggctagatctctagag
ttgcacttcacccaactccagcaagaagggggcggaaacctgagggtggaggtactgtgg
gaagcctgttggcatagacagacatcctgtctttctgtttcctgaccactgatatgacac
catgataagagtttctctcctgtgtcacgtcctcctgccccaagtcagagggaatccctc
tttaaaatgatttctgccatgtatttttagtggtggaaaaagcctgttaagaccgtgagt
tttatttgtgttgtttctattctctgggctgatttggtttgtcttccttcatgtcagaat
cacactgttaaatgagaacagttctaatgtgtctttgaaatttaataaagtgaaccttct
ctgaaactttcaggtcagcttatcaagtttcaaacatccttccaaaataatctaatatca
agcaatttctaacaactcttggaattatgattggaacctcactgaatttacagttttcag
tgcggtaacagtcatctgttcctttatgtacttttaaatgtcttctggtacagttgatgg
ctttcatagacataatctttagatttactcttgttactgtagctctggctggcccagaac
ttgctgtatagactaggctggcctcaagctcagacatccacttgcctttgcctccaaagt
gctggaattataggtgttgccaccatgccccatttctagttccacctccccatttttgtc
ccccccaaaacttttaattcaaaaaataggttctttcgggtatcctctgttgataagcct
ataattagcaaatgctgcattaatgctgcccaagtctgttccttttaatttttcttgtac
taacaacattgaagcccttccttgttcttggtcatttttatgctagtgtgtggaagagag
gtagatttgatttttaaggaccaaaaagtcttaactatagtccttataaaggttaaatat
ctagttccaacaagcaacctcttatagggtagacactttgttcctttgtagtcttgttag
ttgtactgacatgtgaagatcactttcatgtcagctgaaagattaatttataagaatggc
tttgatatatatatatgtgtgtatatatatatatatatatatatatatatatatatatat
atatatatacacgcacacacacacacacacatatcccttcttaatggggcttctgaggga
ggctattcagaactggaaatgtactcagtatttaatatatttctgtaactgaaactactg
taactgaaactactctgtttggatgcttacggtaacctgaatccttcttgatgtgtgagt
tgtcttatactttgttataacatggcttaagagaataacttgataaaccactttatactt
catgctgtttccttcatcagtagaagtatcatcataccttaccatctaaatggctactag
aattaagtaattcccagaaggcattcagaagcgtctgatccctgggattggccgtctatg
ggctgccacctactgcctttctggaaatttctttaaattctagaaagatactagtacaac
tgcatttccagccctatagtaactcttcttggcttagtacttgcatggatgactagctag
tgttctgggttttactttctgtgatttttttttttcccctcctctgcagcctattctggt
tggcctggaaattatgttaatgattgttactcctaggggctggagagatggctcagaggt
taagagttcaatttccagcaaccacatggtggctcacaaccatctgtaatgggatctgat
gcctttttctggagtctctgaagacagctacagtgtacttgtcatatatataaaaaataa
gatttaagattttatttatttaaaaaaaaagattgttacacctagttaatcactgtgtct
ttcaaggaagccaggttagttgctgccactgtagctactgtactttctgtgcgttagctt
gctctgtttccctttcctattttaacttgttttctttacttttatactacttaaatgctg
cagtagaaagccaccccaatgggtacacatatacacatgtgtatatatgtacatacatgt
gtgtgtgtatgtatgtatgcatctaaactttagattataaatgtgtacttgggtatattt
ttgtaagtttaagattatagaaactgctataaattcatgggcatcaacaagtagatgttg
gcatagcttggctgcctgatgcagtgcttcttctgttgactgcttcccttggccccagta
cctaccattcagtcatttgggctctgtaggctcccacaatgtctctttctacagaaggtt
ttgttctcactttctagggaagacaggactccgtggtgttccttagtgtcttccttctta
ctgggataggctcccttcctctccacagccgggcatgctctgctgctctgcgtcattctt
cactgttaagccaaagttaaagttattttcacttagctttttccttcaatttagttatta
aaaaaattaaatttgcttgattcttggttctagactattctctcctgatttttttttttc
tctttccaggcattcgcaatatcatgactccatgcagctatcttagaagtagatcagaca
aaacaagtctaaatctgagttagcagtcctcactcacataatttcacctttagcctactg
tctcagcctattctcatggggcttcactgtctgtcttgtctgtctctctctctttcttat
aactattctggccttatgctctacatgtactatatttttaatgtagaaaaagatactcat
gtcagtcattagggaggtgaaagtttagcctgcaagatagccatcaaaaaatagccagaa
gggctaaaattccaaggactcacaacagagatttccttgggaagataccccaggaccctt
ttagatgacacctctggcagcctcttacaaattgaaacatgtactactactggacattgt
cctcctatgtattccaaagagaaatgtaaacactcattcataggaagctctttgaaagga
aggcgtactagatttattcagaatatctgactactggaaacaacccaaatatctttcagt
agctaatggctggagtttcctgcagtggaatgctactcattaataaaaaggaactcccag
tgtaaaaagagacatagatgaatagcacagacacactcagcagaagaacctggatacaga
attctataatttctcttatattaagttttagataagacagagctactctacgataaaaag
taaaaattatgaccactgactgcctttgagtttaactatgctagtggtggatagttgact
ggatatgtttgctaaagtgatacaaatgttcagtgtctgactggtatatgtgtgcgcgtg
catgcgttacagggtatatttatttgttggacagctaagatatgtgtgtattttctggta
tggatgatttatctagtgattataaaaatagttgaatgactgggagtgggcggcaaagat
gcttgaaatttccacttaagatttttgaaggtcaaatgttataaagatataaacaacaaa
tacccgtgcatgcatttatattcttcaataaaaataaccttattcatatgataaattcct
ttctcttaggcttctatactgactccttactcttcagtccctttttgctgtttttttttt
ttttttttttttttttgaggcagagttttactgtgtagcccaggctggccttgaagttga
agtccttctgctttaagcgctagaatgcaggtatgttccaacctgcccaccctgttctct
gctttaaacatctgtatgtttgttgaaaggtcattgccatgggggaaagctgctcttgat
ttagaacattcactagggtgtgccctttcatagtttatcacagttatggttggaattaaa
gtgacccttgcaggttcatgttttgaaatcctgttcccaagctggcgctgtatggcaaat
gggcctgatcggtggaggttggtcactaggagtggtcttgaaggttacagcccggactct
gcttctgggactttgtacttccttgtctgttgtaatgtgaggaatctctggcacaggctt
ctgccgacaggaactcctctccacccttcctgcagctgtgcatggggatctccacccttc
ctgcagcagtgcatgcagaccatttaaattgtgcgctcagtaggtccttccttgtgtagt
ttctgtcaggtgttttgtcagggcgagggacgagacaagtggttaatacaattacatgct
ctctccagaagacttgagtttgcagaggcagagctgttctagtattgctactactcaggc
tttttacatcctagtgaacagttcttgagaaaagcaagtaaggaacacatctctctgtat
gtgtgtgtgtaagtatatgtgtacatgtgtgtgcatgtatgtatgtatatgactacactt
tatttcagctcctgattttagagacttgtgttgattatgatgaggagggtatagcacagc
aaagcatgaacatcacaacagccttggaaggacatagaaggagatccagtggggcaaggg
caagtgatagccccaggttcacactccagtgattgatttattccagtagacccgcctcct
actcatcaccacctctcagcaatacagtcacgagatgaatccatcaggagattcatacat
tgattaggtcagagccctcagaatccagacacttctgaaaatctgtcagcagacaaccaa
gcccctaatactttagactgggatgggaggggagatgaggcattacatactcaaactaca
gtgcattttcatgctggggtatgtatatataactctcttcccccacaaaccctatcttgc
ctatagctctcatttgcttttgtcacttaacaaaacaccacagacatctgctttatgatt
accttcattcaaaacacagttcaggctggagttggctcaatggttccgagcgagcgcttg
ctgctcttccagtagacatgactttggttcctagcactcacatcaggcagctcactgcag
tctgtaactgcagttccaagggatacaatgttctcttctagcctttgtgggcacctgcat
agaactgcatgtaacatacacacttgcacataattagaaaaacaaagcagttctgtcgct
tgattttaccagatattcactttttatctatgaaatgtgaatactaacagtttctgtctt
ttaggacttggagagcataatattattactatttcttaatgaggcatgcaaagttcccaa
ttttaagtgttcagcatgtattgaagaaatgttggcagaactagccattttgttccttta
aatttttttctcatatactacattttgattgtatcctttcctttgagatttttcccacct
cacttcatgctctcactttctctctttctctctctcagaaagtaaaagcaaatggtcaaa
taaaacccagtaagataaaaaaagtaacaaagcaaagaaaaagcataggattgccccaaa
tccaaaccaaacccctaaaccagcctgtaaagtctgttttgtgttggccaactactcctg
ggcttggttatatatcaattgtaactatcttcttggttaagggtggggtcacttcccctt
ctctttgctccagtgttgtctgggtccaacttcttgtgagcattgtgcatgctgttaagt
ctctgagctcatatgttttatcagccttgttattttggaagatgctgtttccttggagtc
acccactgcctctggctcttatgatctttccatattcttttacacattgatccttaagcc
tgatttccaagccgtctgtgttgactgtgggttcctggtagagagctggtttagggatca
gttgggaagctggtccctaaaaggggttactgaatgggataacaaaaagaacgaggattt
taaacatttttaatgtagacagtcaggtggcagttatattttgcaacaatcacttttatg
taattatttttataatcattcttcccagcagaatgctttaccagcagaataattcctcac
accttccccacagccatttgtgacttctgttgtgcagtctctgtgaacttgagggttcca
ggtagcacatatacatgcatttataccgtggttgtctctgtatgactggcttatagccgt
gtggcataatgctttcaaggttaatccatgtcttaccatgtgttgtcacactgatttaca
acttccctccttgtgattggagaagacactttcccagtggatgtgaaaacatgtccatag
agttttcccaagaaaattgataataaatttattaaaaatagctaacgagtagacatgtac
cagtaccatggacattcatagtaagattataggaaggtaaccatgatcaaggttatatgt
tctgagtgctgggctggcaagatggtttagcaggtgagaatacctgctggcaagcctggt
gacgtgactcctgaaacccataaagtgttagaacagaatcagctcctgagagttgttctc
tgacctgcacatgcttgccattgtgtgtcctctaacaataaaatataataaaataaaaca
ctcagtgttttctattgaatttagaagtagcagctttgtatcttacagttctcaggaact
tgaaggattgaagatacctgatattgtagtcatttcatgtattaaacaagtagtcatatt
gatcatgaattgcatttaaaaatccagccttcatgttcttcacgtaccagtggtgcctca
tttccccaggtctttatgccttctgaaggcattggaaatagagcctttagggctggagag
atggcccactagttacagagcgtacgtttgctcttgcagagggtcggagttctactgtag
cacctacattgaggggctcacaacttcttgtaactccagctctaggtgcatttcataaca
taactctggcctctcagggtgcctgcacttgtgtatgttgcatttgtgcacacacaacca
catacataaatacacacaaaccttttaaaatcataaaagaaaaggagaggttttggtagt
tcagatagttgagaggtcttttgggggtgggggtgctggggatggttcacagcagtgacc
tttctgaataacaaaccatagtgcttcttacaaccatgtagttaattgcccattgtctga
aaccacgtccagattctagagtaactagacacttcttcatgaaccaacagctattttcca
atataataattcctaaagctagttccctatatggatggttacacttagtcttactaatgt
attaaaagtctggttgtggcatttgattttatatattttatatttactttgtatatatgc
tgttcacataacttacagtgtcctttctttagtctcttctatttgttcaagcttaattat
acctgttaaatcatatttagtagttaacattatcatattgtaatttactttagttttcct
aggtgtacatcttattctccagaataaattgcttgaggtcgaagaggagttagtttggca
gtattttctgtgctgtgctagctgaagctgacagtgctgattggaagtgatcagttatgc
ttttgattttgtgttgtgttgtttctgagatatttagtaggtagcatggattttatactt
tggtcttatgggtaaccacaacagaataaagtatatattagttaaattctcaacatctct
tataattggatgtattgtagagagaagatttgagtttagtgaggttttaaagtaaatgcc
ttaaagaaaacctcaagttcatatgataaaacagagaaacatttcaaaattgaagtccct
gaagcttctgatgtaagttgaacaaatgttgcttccgcagccacagcaatagaggagtag
ctttattggctgaggcacattagtagctttgtcgatgagaagagctaagggtccttattt
caattcatggttatgctagagtagtgaagtgagagctgttgacctgctcttaagcagaga
ctagaaagtttttacaaatagattaaagtacatagagaggtgtgaggagctaggcatgcc
tttaacattaggaatttcatccttttttgtccagtcttaatacagttgaccctacaacac
ttactaattgattctgaaaacaacagctttaatgaggtggtttaatagagttaaggtaat
agttgagttgttggtacatctctgagtgctattggatacaaagagtaatatgagatacag
gatttattaaactggagacaatgtaatgccactaggatggcacatctccctatttgtttt
atgagtccgtgggcatgactatgtggaggtcaggggacaatacttggagaagctggctct
gtgtttccatcatgcagacccaggggattgaactcaggttgtcaggcttggcagcaagca
tcttcatctacactgagccgcccttttggtccttgaactgtaacttacctgttaggtttt
ttttttttttttggttttctttctttttttttttttttttttaaattttactttcataat
tttcaattgtgcgtattatggatgtttttctacattgtcaggttgttattttttcctcat
ttctttttattctctacttttcatatttttcagttctctcatgtttttattcttaactca
tgatatttttcttaattttactttcctggagaaggctatcctagtagaggttataattgc
catggtgaagtactataactaagaacaatttggagaggaaagggtttatttggcttttgt
gttctgaatcaaagtccactgatggaagcccaggcaggaactcaaataggatagaaactg
tggagacaggagctgatgtagaggctttagagggatgttctttgctggcttgcttcccct
ggcttgctcagtctgctttcttatagaacttaggaccaccagcctagagctggtcccacc
cactgtgggatgggccctcctctattaatcagaaagccctgtcagtgcttgccaacagca
agccatcttctggaggcattttctctgttgaggctccctcctcttagatgattacagctg
tgtcaagttggcctaaaactgacaggacgagaatcagctcctaattcgtttcttgctttg
tcttacccattttttatttgtcttttgtatttttagttaaaggtggaaaaaaaaaaactt
ttaaatgtttacttcagtcattccagttgatcttactttattgtgttttctgtggctttt
gggttttaaacttagctgaagtttgacttggattatcttgagtacttctgcttctgttcc
ttagagtcctacaagtgggagctgggaagaaggagggctcagtggttaagagcacatact
gcttttctggagaccagagtgaggtttccaggatccatatcaggctcacaacagcctgga
actccattcccagaagacccaacactcttttggcttccaaggataacccctgcccacaat
gtgcatgtatagataagtaaaaaacccaccaatttttgagtaccatttaaatgtgatata
ctaaaactttttaaaaatagaaaatagaattgttttaagttttttttttaatttagaaga
aattttaattaaaaaagtacatatgtttatggagttccatgagatgttatgatacatata
tgcactgtgtgatgtttatggtaaggttaatgcatcaacctctataaacagttaccattt
ctttgtagtgaaaaactcaaaatctagcttttttttttttttttttaataggttataatg
ttgttctgtgtaatgattaggctactgtgcagagcactggaacttctagctctgagctag
agcctgggaaagtgctggcttaggtatcagtgtttgctggttaccatgcaggaagaatct
catgaacttactgttgaacagtgtgtagctgcttaggtaactgtgattttctgctttagt
ggacattccatgcttcttccatccttacagtctttctgatctctctgtgttctcagaaag
cataaaactgggtacttactccttaacaaacccataatctgcttctgtgggctctttaga
taatgcatgatctgttttttctcttgcctttggtcattgtagactcttaacttcaaactt
caaagtagatacctctccccctcccctcccctccccatcctcccctcccctccccatcct
cccttccccatcctcccctcccttccccatcctcccctccccatccttcctcccctcccc
tcccctcccctcccctcccctcccctcccctcccctcccctcccctgcctgccttccttc
ttccttcctgaaggatagatctctttggtgtaagagttcagtcaggatgacaaatctctt
attatgaaggagtttcccctttattgttataagatacctctccaattaacatttacatta
aaggaggctgatatttctcttaataattgagagacaactttaaagcagtatggttcatgc
ttggagggggaatgcagtctgacttaggaattgatgcagtggaagtgttcctgtggcttt
gctctaagtgttggcatctgtattgtgtggctgtcttgtcttctcacagaaccctcataa
ctcctggctgattgagctttctctacaggtcaaattcttatattcttgtaacattttggt
gagtctacctctaatgaatgctggccttaggagaaacttaagtttctcagcatctgggga
gtgctgagagagcttatctttaaggactgggttttcaaacaagttggtttgtaaatggtt
aaaggtgcttgctgccaagtctgccaatctgagttcaatctatatgacctacatggtgga
ctaagagaatatacactgggaattgttctctgatctccatgtgtatctgtggaacttgtg
tgcatttgtatacatgtacgtgcacacattaaacaaaatgttaaaagaaagcaaaacgtg
tcacagtccatggcaatctgctgttgtgaattacaaagtgtaggtggctttttttctcaa
atgtgttaaaaacttcaaattgaatcaggatgctaatttagtttagagagaaatagaaat
tgtttacattaagagacaaatggaaaagtaattaaaaagaaatgagactgcttatatttt
aaataaagaagatactattgtcaatattatgtagaattttagggatatcagaatcttctt
ttgtaaagcattcttcagcaggtccaaaagactttcttaaaaaagacattggttagaaaa
aattcagtttgtaaaacaataggactgggtagttttctttaaaggttttacaggtccctg
gcacgaccaccatctgttcactgctaattctttatatgtgtgctggattatatatttgta
attttgtttgtgtgcatatgtgcatgccacagtgcatgtttgtgtggagctcagaagaca
atgttcaggagttggttctaactttctactgtggattctggggatcaaataagccaggct
agtaaagttctcacttgctaagctatcttaatggcccatatgtttgtggattttacctga
acctgttctaaggtagacctgggggaagggtgtgggggtagggaggagataagatatggg
gcataaataattaattaaaaaatgtattagcaaaatttggtgttaagtggtagagctgta
gagtgcttgccttgcatacatggcatcctaggttcactttgtccccacaagaaaccattg
acaaatagagctagggaggtcgctcagtggatcagagtgccttatagcacaaacataacc
agtagtgtctgtgtacccttgtgaccctagtgcttggggcaaaggtcagttggaggcaga
agaatggctggggcttgctggcctattagtttagccaaaaaagtagcaagctctaggttc
agtgagggaccctgtcttaaggggctaagggagagttgacagagaggatacctaccaggc
tgccagattttcagatcagagttttctatgtcactagtctgatatatcgtctaaacttgt
tttattgaaaacaaatttgtcaagtctgttttaggaaagtaaacttgaaaaatggaacta
tagttgaacgttctattttttatttgttgctatattttcacaaagtgaaacccagcctag
atagatggagatccattcttcaggagactcctgtgcctttcactagatagtagctccttc
ctaagagaggtaatcacctgtgactttatcagagatatactttgcttagcctcatttttt
tttttttttttaaatagccagatcctaagtgtgtgcttgtttaggttcttatttcatatt
cttaatttagtttctgttatatgactatactgcagtttatggcccctgctgctgttgata
cttggattctgtacagtgtggggttcataaggataattttgctatgacagttcatcttcc
tatctttttgtatgctctcactcactgttggtagtagatgcctaggagaggtgttatttg
gtcagtaggagtgtgtatctctgtaccattttatgtttctttttccagaggataaaaatt
acagttgcagcacatgcttgcttgcaattaatattatgtagttttaaaataaacaaaata
ttttggtatgcagtaatatctcactgtgataacaatctgtatcttcctgattaggttgaa
tatgatctttagaatttttattagattcttaggctatcttaaaaattagattgccatttt
attttaaaaaccattccacttttgattatgtatatacatatatgtgtctgtgcgtgggtt
gtgcatgtgagtgcaggtgctcatagagtgagtacaggaggatatggaactcatagctag
agttataagggggggggcactgagctaccgtgggtgctgggaactgaactgaggtgctct
gcaaaagcaggaagtgccctgaccacagagctgtctctctagcatcagttgtcattttct
ttctttcttttttttttttttttaaagatttatttattactatatgtaaatacactgtag
ctgtcttcaggcacaccagaagagggagtcagatcttgttacggatggttgtgagccacc
atgtggttgctgggacttgaactctggaccttcggaagagccgtcgggtgctcttactca
ctgagccatctcaccagcccagttgtcattttcttaatgatttgaaattgttttcagcat
gttttggatatgaatcatggttgtacatgtgccttgccagagtactgtatccttagcctg
cattttcagtttttcataatgaatatggaattataccaatctagtttttttccacgtgtg
ccagttaggaaatctttgcccaatttgaaaaagttataaccttttctggtcatcttctaa
aaactttatggtcctaaattttctgtttaagcttctaatagccccctttctttctatatt
tcccacggtctggggttagatgctatcaatctaattgctcttgattttagcacaagtaga
gagaggaaatctattgatatgacttaatatcatttacatttatttaatttggtgtgcatg
tatgtatgtgctgggtgcatacatgtggcagtcagaggacaacaggataggttccttcat
cctcgtgggctctagagatgaactgaggtcatcagacttggcttctttacctcaggaccc
atcttgccagctcaagaaacattttgctgccaggcatggtgttgcacgcctttaatccca
gcacttggtaggcagaggcaggcggatttctgagttcgaggccagcctggtctacaaagt
gagttccaggacagccagggctacagagaaaccctgactcgaaaaaaccaaaaaccaaaa
caaaaccaaaacaaaaaaaccaaaacaaacaaaaaaagaaacattttgcttttgtataag
ttttagatggtagcagttgtgttgttaattaaatctggtgagtctgtaaaaggaatttta
aaagtttttccactttgattaagaggcagccagttatactttatattatagtacatcctt
ttgagtctagtaatggtggtgatagaagacgtgtgttttcttttgaggtagtgtctctag
cccaggctggccttgagcttcctattgaactcctgattttcctgtgtgtacttctaggaa
cctgtacccagcttataatggtagtgagaacacgaactgtgcatcttggtgtggctgctg
tgtgtaagcaggtgacttagtcgctgttaggcatgaagtacttaacaacaaaaagataac
tatatgctttgtaaaggcccaagtgaagaacctgacaaatagtgagtttataactcttat
tcctgaatttttgaacctaattaaagttgaaacctgactgagaagatttgtactctagtt
gttcttattaagatgacttaaatctaatttttataagtagtaaaatcaacttgccagttt
atacatatctttttgctatggataatatattttagtaaagccgggcgtggtggcgcacac
ctttaatcccagcacttgggaggcagaggcaggcggatttctgagttcgaggccagcctg
gtctacaaagtgagttccaggacagccagggctacacagagaaaccctgtctcgaaaaaa
acaaacaaacaaaaagcccccccccccagggtttaaactactaatatatatatatattat
atatatattaaactttgcatgtgtttttgtagtttattatctcttccatgcattatgtat
acaagttatttttaagatcataaaaactttaggcagactatgtttaactctgacattgaa
taattatttttattttctattattcaggaaatctcagctatcaaggagacttttaaaagg
ctctctaaatatttggtaagtgcttgatttctgtgtacttttgcatgtttcttatattta
tgtatatgctacatgaacccctcttgacagcagtaagttaaattacaataagaattatat
ttgttttcatttttacctaggtacgagaatttcttctctttatatcttttgtgatccctc
tacacattgtgtctagacaatcaggttagtaagtaggaatagtaatttatgaattaaata
agactttttctacctgagattttataataattgtctattagttgccaaaacaaacagatg
atgatgatgttgtttactgtttgttgctgactgttccttaatgtattaagtcttgtttat
tacaatgtatgagcataagcaccgaccggtcctgtgggttggtgtctatgctgtttccca
tctgaattaccttaatttctctgttctaatattaaaacgaaaacttctgtttgaagttgc
ctctaattaagaatttggtaagttaatcatttcactagggtaagctatcccctcctacta
gccccttgtgtacatagtatgtgtatgcggtgtatgtatgtgtgcacatgtgagtacagg
tatgtatgtaggcatgcatgctgaaaccagaggaggatgtcaggtgtcttgctttgtcac
cttcttccttcttctcttgagacagagtctcagtgaaactcaggttcaccatcttttggc
cgagctagctggccatcaagccctaacagctttttgtgtccctcaacccagtggtgacgt
tccagggccacttggccatacctaccttttatgtgcgtgctgggaatcaaaacccaggtt
ctcatgcttgcagagcaagcaccgttagtcagaagccatttccccagcccatctcttgtc
cctgcctcttttaggctggttcctgcctgtccatccatgcatccaccatacatgtatgat
gcatatcctgcacataggggaaagcaaagcaggtgatagtttgtcttccctgagtttagc
ataatcattagttaacatgatgatttcatttctatctatcttcttaatgtatttttattc
ttctttaatagctgaggggaaaagaccccctcatagtttatgtgtgcatgtttctccatc
cattcattcatctgttgcatggtggctgtgaacagtgctgcagcaagcactcatgtgcac
agcactgtgctgtgctggctgcttcttttagctatgtgtctaggagtggaataactggaa
cacaaggagttctagttgtaggtttttttgaggatcctctgtactgatttccatagtact
tataccagcttatatgtatactaacagtgttgaaagcattcctttttactagtgaaaaaa
attcaaaaggattaagtgttttataaaatgggataagtgctgagatatccaataatcaag
taccgtggtatagtttatggttgtttttaatataaccacacaaaaatgttttgttttgtt
tttcattcgctaaaggagtgtctctagatatatgaagtgggaaaaacatctgtatttgaa
acagtcagttttcaaccaaaagatatttctatagcaattatagcacatgcttacatgagc
agcagttacagtattctgtttagcatacacctgcaatttgtagcatattcaggaacaact
aatagagcaaagatgcctgtgaagatttccactctgtgtgccttgtggtggctcatctct
ggagtatatcacacagaaggggctagtttggccctctcctaaacatgaaccagctgtcat
cattgtaggcttttggagcatatctcatcctaaatgtatttgtgacagttttattgtctt
acagtttacacaccagaaaacgtcacacttgtgtactttccaggattctgagtctgtcag
ctatgcactcctcacagccactcaggcgtagggggctttccttactgtctaacctcatct
ccctctcttggcaacaagtaatgggctttctgctcgagagtttgcctgttttggttattt
ataaaaatgcaccacttagtatttgttcttttgtgcttcattcacttagcacagtatttt
tagcatcatatgatatgtgtcatatcagtacttaattcatttttatgacttggtattcct
ttgtatcccacatgttacttatcctttcatcagttgatagagctgcttttttggttgtta
gtaatgtgtattgtgaactagtgtggatatatattttcacttcttttgagcatgtgtgaa
tggggaggggttgagaattaatcctttgtgtttcttttcaagtagtggtcaaaatagcaa
tacactggggtcctagtctccacatccgcatgggcagccatgtgtgtattttagtcttcc
tggttggtgtgaaaggacacttcattgtgggtgcttactggccgtttgttgtctctgaag
aaagagccattcaagtattttgctcattttccagagcaaatacttttaattttgaattgt
gatcattttttttacgtgttatgagtagtagatgtttattcagtatgagagttccaagta
ttttcttcttttctatggattatccttttagtttgaaaaaaaaagtcattggaagaaatg
acccattttatcagatgggtttctctgtagccctggctgtcctggaactcactctgtaga
ccaggctggcctcgacctcagagatctaactgctctgcctcctgaatgctgggtttaaag
gtcgtgacaccattgcctggcagaatgaagtgaaaggttttaatcttgatagagctcagc
tttcattttgctttgcgtgtggcacaaagttacacgttcattttaaaataagtcattatt
gatagtttaggtctgtaataggcttcaccctaagtccataaagaacactggcatttcttg
tatctgacggccagagcacaaagggaaggatttctactgtatttgtctttcagttcccca
tgcatttgtttcttatgtttgactctgtgattactcaatacccgaccctacctgcccttg
ggggttggaggttaggtagtaaagagtcaacaacacaggctccaggagcaggtgagaaac
accgcagcaagctcttctgaaaactgctgcaagctcctttaataccctctacacacacat
gcccattggtcccaagaacagcagttcctgactggctggcactgtctgcatcaatccttg
ctctctcaggcagtccgcacttaggtcctcctgcaggatatgcttttgcggctccttgcc
ccctcccggcatttacataaaggtgaccccgctggtcctgctacagttcctatatgagat
tggtttatttatattagtctgtgttgtatatgatagctccccctccacctgagatgagac
agggtcttgtgcatctctggcagatcctggagatctctgcttcctaagtactggggtttt
tgaagttttctttgcatgcctttggtttctgttctggaaagaaaatagattcataggaga
cttatctttgttactttttggctgagaatgttcttctgatttggcatgtggccagaagct
ttctgagagcctcacagaacacaaagctgatagaagaagtgctgggaagaagctctgagg
caagatggcatcagaggatgtgagccatgtatctgagtacagtctctgcccagttctttc
actgactaccataaactactcaggtgccagggtatcaggctgataaataggaaggcgtgg
ctcaaaggtgaaggagctgagcacaggcagtagctacatgctgcaaaggacagtttggtg
ttcccattcacaacactaaaaccacaagttcctctgaaagattatagcaaagtccagata
gactcttacattgtttacataaggaattctgttttcaaagggctcaagaagaatacttag
gagtgaatagataaagcatagaggcactttaaagatgcaggagagagttttagggctgtc
tggaaggagagttaattaagtaacaaaacaagtgtttagcagtgaggagagtcaattagc
ttagagtaataaaagccatcaactggaaggatgggaggatgaagagtccaagtcagaggc
ctgcgagaggattgctagcctgtgatggcacaggaccataaataatggctgcagactcaa
ttcagggggaagtgtcagcctacaaaagcaggaagttcaggcagaatcaacataaaagag
tccacatttagctaagtaagtggtctagctgcttagttgcagagaaaatcttgtaagcac
taatagagaaagaagttatcatgttggctagttgaggaactagacattcagtgagctgaa
agaaaacctaaaagatgcgaatttgttaccgtaatttgaaagtaaaataaagacatgttc
agataaacactggggttttctatcatcccaccacacttatgaacagttctataggaaatt
gattggtttgaagggaaatagccccagaagtcatatcgaagctagtaaatacataatgta
ctatatttttctaattataaaaagctatatgatttccttactgtaaaaatgactaactat
aatactatattgttgactaatgaaaacatttatcaaattatactgtttctttttgactaa
atttggatttttttgttgtcgttgaggatttttattgtgcttttgcttatttgaatgtat
ctcctgtgaaatgtctgagaaatgacctattagactctttaataagatataccatgacaa
gtggatgttaaatgatatttagtctttcaacatctcaaatctgctaggtctttaatttaa
tttcaggttgtttaatagcatattgtcactttgaaatgtgagaattttctaacaaatctg
acattgtaattatttcaggtaggccattcagttgattgcatttacactgcagtccatatg
cctttcactgcctgtatgcagaactttctccttactcccgccagaaactctgttcccagt
aagcaacaatcccgtagttcctcttctttaagtgttattagaccgtctgtctgagccatg
agagtgctgtaaattctcaacacgagctgctcagacagctgtttgcagactcttctcagt
ttgtgactgattcttcagaaatataaaagctttgctgctgaggcctcagaaggaggtaag
ggaacctgggagagaaagcgggaattggggctagagagatgatagaagaggtgaggactc
ttgctactcttgtagagactggggtttatctccctgcaccaacattaggcagctcgaaac
ttcctctaccctcctctcagtgccccttaagacatacatgcatctgcccatacaaacaca
catgcacagcactagcatgctcacacacaaataagtaaataagaatacttaaagagaaga
aagctttttttttttttttttttaaaggaatcaatgaaatgctcatgaatagactgctgg
tggagatgtaaatgttaaaggttggtgagggtcacacgtgtggaggacacagggcctcac
tgaatgtagactttcccagtgggttagactggctggctagtgagcccctcatgcttctgc
ttctacctcctagtctgggatcgccccaggattgccattacatgctagcctttttacatg
gggagctgaactcaggtctccatgcttggacagcaagcattttgcctacctagccatccc
cttagcccctaaatttatagttttcataaaatgtcagtatcacttgaaacaaacttcttt
tagagctggggaccaaacctgaggatttgccagctctaccactgaggtgtttccctaacc
ctgtgtgcttaggagttaccataaagactttgtcacttctttttgaacacatgaacattg
tgcagtttaatgcaaatttcagaacttccttgttaaaaattgaatttatgatagaaattt
atttagcttaatatattagtctacttagattagcttatttattgaggaaaattatttgga
aactggaagtacattgttttttggatcattttaattggtaacttctgataatggcagtga
gtacatttgaattataaaatctatacagtcagcccttatctacctttaaatcctttacag
tgtggtcttgtttgcttgttttttgatggaatcatatgcaaggatataattgttgtgttt
ttagccattactacattgaaattcagtgtctaggtttaaagtaaaatgtaatgttactta
gtatgaaattactaaacattacagttccctcttctgtcacttcagagttactgcaccatg
cttagctttaaatagctgaccagaaaactgtgtaccaattgtattgttagtatttcttta
atagttgaatttagaatattcactgccaaaagttatttgtaggtaatatgggagattggg
taacaaggcaagtaacacagtgaaaaacacaggcattgtacaattgatggcatcagcatg
tttctgctggactgggcagggcatctaggcacccagcggggtggattaaggcgaaggaag
aagccggtggaacggagtgctactccattcatctgcgcggacttctttactttagatgtt
tttgagacaatgtggaaaatttgactattgactggtgttaatgaaatgtggttaatgtga
tctaattgtgtggtgattctgtgaacatgagaggcacttgtgaagtgtgtgtgtgtgtgt
gtgtgtgtgtgtgtgtgtgtgtagtaagtaggcatggtatataggacttaatttaaaata
tctgcaaggaagaagaaaagtcaaagcaatattggtgatattttgaattgaatataggtg
taatagatataaaatataaataatatactgtttttttaatgtatattggaaacttttcat
gtaaaaattccagatttttacctactgactgttttcctggtaccacaaatcctctcctag
catttacaagtcttctgagatattggagagctttacttaaatttttgttgcagaaatgag
gctcttgtaaaccatttaacctttccctttgtattttcttcttgttatctgtcaccattg
atcatagtaattattctggttgttacgtcttttttttttttaaactttattttgtctttg
ttttgggaatataaatttaattctggtggaagagatttgtttctgttgcagtaccttcat
tacaattgttttgttttgtttttcgagacagggtttctctgtatagccctggctgtcctg
gaactcactttgtagaccaggctggcctcgaactcagaaatccacctgcctctgcctccc
aagtgctgggattaaaggtgtgtgccaccacgcccggctccttcattactcttagttgcc
tagcatattagatgcttaataaatatttcaatgattgagagaatgggaattagttctaga
aatccaaggacatggagtttggtcaaggacagagcaaaagagaatgtttttgaatcttat
gaggctatggaaaccttaacctttggagaaaaatgtgggctaatatgtacagatttggaa
gagtaagtcaccacctcttgttttttcactctaaaaccttgatatttcatacattcagca
tagtcccaacaacaggaattcctctaagttattgtgctgtgcttttccaattgtcttcaa
aatgttctctgactttttgaggcaaatcttactctgatttccaacttaacatggaagcgg
tcctcctgctcagcctctcagctctccaggacttcctgcctgtgttgccatgactgtact
tatttatctgtatgatgtaccacttgagtctggtgtctatgcaggccagaagaagggatt
agtacagatggttgtgagctgccatgtgggtgctggaaattgtacctgggtcctctggac
gaacagaacagccagtgcatctctctagccttgaataactaactaactaactaactaact
aactaactaactaacacacacacacacacacacacacaaaataactatattctccttaaa
caaaataagaaggatggcattgtgtttcatttttgtaaatgtatttaaggtctggcttga
aatatgttttagcttttgtgtttagccaagtgagagacaggctgtaaagtgtaaggaaac
ttctcaggtaaataactacaggtgtcttcttataacatagattgagaatgacagactaag
caacatcctaggagtactgaaaacagttgtggtctcatagattccatgcaaggatcctgg
gaaccctccatgttgctggagtgaataatttctcactgggatactgtttatgaacaacat
gctatgtttgtaaatggaagaatcattgtttaaagatatactagaaccggtttggtgaat
ttatatttgagacatttttcttaaagactgttgacagtaccttatgtttacttacacaga
taaaaatgggccaaagaatgtcctgccacaaagttgtgtttgtagttttctgaggatgga
tgaatggactgatggacagatgggtggatggagggagcaggggagggagctggagatcta
accaaggcccttgttcatgctaggcaaacactcaagacctctactacatacagtatttaa
cttctcaaaggaagaatttgacattgagttacttatagttaagaaggaaactagttactt
tattgaagtaatcatgagggctggggatgtggttatgatggcttgaatgagaatggcccc
caatgattcatatatttgaaagtttggtccttagtaggtgaaactgcttaggaagggtta
ggtggtgtgggttttgttgaaggagctgtcactaggggttgggctctgaggtttcaaaag
ccaagactaggaccagtgtctctctctgcctcaaacctgttgacaagatataatctgtca
gctccttttccagggccatgcctgcctgctgccatgcttcatgttgtagtagttatgagc
tcaccacctgaaactaagcaagcctccagttaagttcttctaaaaagttgttttggtcct
tatgtcctcagtgcagtagaacagtaactaagacaacggtatattccttgcttaccatgc
ttaaagccctgggttccatccttagcatcacacaggaaagaaaaatcatacttttaactt
attattgataaattattaataaataatgttttctctactttttaaaagggcctgtaggta
gacttgattaaatatgcactgtctttttccctccagaacagtattatgctcatttttctg
atagttaataaatgtttacttctgttggttagtcttcaagttctgtctgtatgcaacata
gtgatcagtattacattgaattagttaacagggaagaaaaatgataaataatgaaataac
cattattgcttacattctatttttgttagaactaatttaagagccaagggatattgggga
tggtatagggaatgaggctgatgaagcatttcatgcttttgggtatttcattttatttta
ttttgtttttcaggacagggtttctctgtgtagccctggctgtcctggaattcactttgt
agaccagggtggcctcgaactcagaaatccgcctgcctctgcctcccgagtgcagggatt
aaaggcgtgcgccactacgcccggtgtggatcttttattttaggcctttgtagaagcagt
agtgatgctgaggctggtcagatacctggctttcattgagagcagcatatatttgctggg
gactttaaattgattcttgctttattttgtacacagtgatgtatgttctgagcatattct
gtacttattggcggagtaaattatgttagatgttaatgcttggtacctgttcttacacta
gcacaatactttatttatttatttatttatttatttatttattttaccttctttagctcc
cctacttcccatatttgctagagaggtttattagcagcagttaagactgtaccctaatgt
ctctgatgagcatctcaaaaggtagcaacatggctaaagcttgcttttacaagttctgtt
gataaatcattttagacacttagtctgatagtcccttgcattttccatgtactgtgttct
ttggcttgtctggcagcagcacaggaaggtagacttaacggcagtccagctcaaatgcat
ctacagatgagaatttatcttctgtttagcatacagtgttttcagcaggcgagtacgatg
ttactgaattattttgcttgtttctttctacagtcgttatttatagaacttttttccccc
cttgtgaagtacatgcctgtttctaggtattctgaaaaaactgatttaagtataacatga
gttaagtctagaaggacagtgcaaggtgaaggcagaaagagtactgactctttttagctc
taaagcagggaagggactgagcgtgggaagggactgagcgtgacacacacacacacacac
acacacacacacacacacacacacacgcagagcagggaagggactgaatgtgagacacac
acatacacagagcctttcaatgcctcaaacataggctcttctatagaaggtattttgtta
tttttagtttcctttttctttttcttaaaggtgtattaaaaagagttgacatcattggta
tctgaatgttcattaaattattttgaatatgattatttaagaagactaacatgtttgtat
taatgtgactctaattctgggaattcagaaagaattataattactggatacaagtaactg
ttatcaggaaaggtagaatcttttaagtataaaattgttagggaatgactaataccttca
gtggtgtttgcttatgttttcagtgtagttttcgggtctgtctactgaaatgcaaatgac
caagggaaagatgcctcgtcagcttaggttccgatgacttttagttattgcaaatagagc
acatagccacatccttttcttctccgtaaacctcagaacacttatgacagcaggcattcc
acagctcaccttgctgcttgtctcattcttacctgtagtgaaactctgcagtagattttc
ttgttatccctcaggtccaagttcagcatgcagagggaatttcttttaacattgtattca
gtcatcctcctgcctctcccaccactattcaaagatttctgctagctactctccacattt
tattatttttaaaaaagctgtaagcccacgttgtctgtttctcgttgggttttagggtga
ctgactgagaacagttctgcctgtaactgcaatagagaaataaagcgagtgggtattctt
actgtgcgtgcttggcaggcaggcgtcttaagtgtagagtcagaggtcctcagttctgag
tggtgagctagaggctgtccagtgatttgctcccgctgtttctcagagcagtgctgcagc
ggttctctccagatcctgaggctttcttcaacagtgtagaacagtgagggtggggctctt
tccggttggctgttgaaaaagcgtccatttacagagagaacaggttggataggctaagct
gaaggcgtaggcaaacagatccaaaactgatggaagacagaaagagtggttagtatgttt
tggggtgtcttgagcttgcttctttgctaagttcttgagctttaatataaacacattgaa
atattaaagcactttacattctttgtgtagtagcctggagacttatgcttttcacagttt
cttggtgatgagtttgtggctgctatcttctatagtaaagttggtttttcatttgttttt
aatttgcttttagtggtgtatctttcagccagagagctgcagaatgagtcagttagccag
ttttagtatatatgggacattactagttgtgattttttaaagccagattctcttttaatg
atcaagtcaatgtgtggctgtggctgcagcaacattatttcttttttaatgaagaggttg
aaccgttcgacttgctgctccgatcttttcccttgtagctatacatcagaacagttgcag
agattttccatcgaatgtcccaggcaactaaaggtaactacatagatttatttcaaataa
gaatacacagttaaaatggctgcattataagttgggccaaatgattaaacataaaagacc
aattaagatgttcttagcagttttcttgaccttgcagtagctatccaatatcattttgtc
atcatcagattgtggcagtggaaatgtactgcagtaattgattttgtaataggatcaggt
gatttaccagcggcactgagaaccacttgcttgctctgagtgtgcagcactctaatgact
gtgtgattgcagcctagagctgaagtgaggtggctggccacccacagtagcagcgtttac
tttagaaatcacaagagtactaggagtttcttgtttgttttctgtgctatcagttttact
tcatgtgttcatgtgaagtcttagaaattacgattttctttttcactcaccagttcataa
aaagtttatcggtttgactaacattaagtataacaaaacacattcttctctctctacttc
tgagctcagcatcaacatactgtgctgctgaccatgaggacacatgtctaaaacataagc
ttttgttaactattgtacattatctgcattaaggaaaatattcttaacacctgcaaatct
catcatgtttcaactcttatctgtatggtaatactgggactagaggaaactattggtcca
gagagttactagaaaattgttttgtgttttattttgtgtttggatttctcagtagtttat
ttagcaaatctggactgtataatttcttggaaatatccagaagaacattaagggcaagtg
taatgtttaatgatactacaaattaataacttggtatccaattttatttaatatccaaaa
tttataaaatggctatgttttaataatgtttgtgcctttaaagcaaataattggttacaa
atatgtgtgtacagagctcattcttgatgcggaatgcacctgctgttgtaggaatgtgag
accctgtgttgagatcctggctgctgagattttgctacaatgatcttttgcttagctttg
ttgtattagtgaaaaaaaaaattacacgctttccctccctcactcctttccttgccagaa
ccaccaactcttgtgcttagtatgtaatagtgttttgtacagaaccacacacacagggaa
aatgacttttctgtcagtccttagatatatttttcctgttagccttactatatttcataa
cagttaaaattagatcttgcagaatcaagaagagttctgccattgcatatgctatgtgag
gcgcttccctttctgataagcccagaggctagttcttaaactgtttgaaggcttggtgtt
tcttgaaaggtgaaaatatgaacagtcatactttggccaaatccagatacttaaaaaggt
aattatagttatgtgttctgttgctaataatgatgcaatatcaacatggagtttgtagtc
tttattcttctttgccaacaacagaaatccctctgtatacattggtaatgtgtacataag
gatgttattactatttgagatttgttcaaggacatgtctaagttcaatattgaaaatgtt
aaatgatgtaccatcaccaaatcaattactgataaatatttcactatggtagtgttcatt
aaagcccctcccccagcattgtacacttgattgagcaaattggaacagaaatatgtccca
gtgtgtgattgcttcttaaaataatgattgtgaaagacacagggcactgttacatcttca
atatgatagggcttcctttagggttaagtgggagatgtatcctgggaagtccttgcccca
gctgggcacactcttccaaagtgcccgagatcaaacacctggattttgtttgactttgtt
acaaaggaatttgatgatcttgacacagttacatatgtatgaacactataagctccctta
aaatcctcattagaaagaaatgggcgtgtcctgtccctttagcactggggttggaagctg
tagaagttggactttaatggaaagaatcagtgtgcatgctgtagggacaactcctttcca
tattcatgcctttctttttgctgagactactgttttatctttttgatatctaatattctc
taaatagcatatttgtagtagcgtttaaaagaccagacctagggctaaattctgacacgt
gatttttttttccctaggcatttgatgataataatgttgttcggtcatttgttcagtatt
tcacatggtagttttcagtggtcagagtttgggaaggcactgttgtttccagtgctccag
caagtgcacctggcaggtaagcttgtggaggcgcctgcctttgcggcttaggatggactg
ttgccttcccagagatggggatgtggtgccgactcactcatccttcaggaacctgtgttt
tggatcatgctgctctctagtgaggactcctttgtcagtcatttcacttgtgagattgga
actctgagtatttttaagacaaagagactgaggcttacaaaggtctcattcctaaaatca
gataactaggaaatagaataactaaggttgaagcactactttaaaatactttctttgaca
acaatgtgataaaacactatattttcttacactttaaaagaattttctttcagttaagcg
taactgcagagatggcaacaaatgtctgaagatactcatgtatccataaactacttcctg
tactatgagtgaattttcaggtgtgggttagattttatcaactgcttctctctcgaatga
cagaaaagcttggcatttactaattgtacagaagaaaaatataagttcatgttaatatgt
gaagaatcaattaacaaacaacgggactagaaagtacagtttgtccttctataaaactat
tatgtagtgtgagttggtttgtgcattcctcactgagggatttagctggtatagtggtaa
agtcacttcagtcccccatgttcttaagtaatgtaaatacatctatttcagggtttctac
tgctgtgataaaacaccatgttcaaaagcaacttggggaggaaaggggttatttcagctt
acagttttacatcacagtccatcactgagggaagtcagaacaggaactcaaagtagaaac
atagaggcaggagttgatgtagaggccatggaaaagtgccactcactatcttgctttcca
tggctttcttataccatctaggcccaccttggggtggtatcaccagcaataagccaggca
ctgagatatcagtcaagaaaatgccctgcagacttatctacaaataaatcctgtggaaac
aaagactgtagcttatgtcaagctgacatgaaaattagccaacacaccatcagaagtaag
tttttttcacaaagagggtattatcaatatataatggcattaagaataaaactgaaggtc
cagagagacacttgctatcatgcccaattacctgagttcattttctacagcccacatgat
aagaaagaaattcagagagactcctgaaattgtcttcagaaccttctgaatgccacatac
ctattttcagaacctgatcttgcagaatttccttctttttcagagaaagacagattgttt
attatttgtgtgaatgtggaacatgtgttatctaagtatggatatgtatagaatgtgtat
acacaggtgtatgcatggagggtattaagtatctttctcagtctatcttacctctatatt
ttgagataaagtctctctattagtagctttccatgtcctgaaactcaatatgtagacaag
gatggcctaagactcagaaatctgctcacctctgcctcccaagtgctgggataaaaggca
tgcaaccaccaccatgcctggctcaagccagtgtttcttactgatcataccaatttagct
acacattttggccagcaagccgcaggaatcctcctatcgccatctctctagtcctgggat
tacagatacataccagggactcaaaacccaggtcctcacactcatgcggacacactttac
ccaccaagccattcaatttgtaccatatgtaacagaactgcctgtgttacagctttcaag
gatctcagggatagccagtgcttcatttagacctggctttactgtggaatgaataccatc
ttccttgagatgaagttttattcctgatgttcatcatgttcagtcaacctcaaatagtgt
ccatttcagctgtagtctaagaaacactaatagagatagaatgaaatattaaaagaaaaa
acactagaaatacagatttaattatagagctaggatctgtggtccttttaaaacaaatta
aatttaaattcttgtatacgctgtctacttttcaaaacaaagtctggggctggagagatg
gcttagtggttaagagcactgactgctctttcagaggtcttgttcaatttccagcaacca
catggtggttcacaaccatcggtaatggaatccaatgcccttttctggtgtgtctgaaga
gagtgacagtatactttgtgtgtgtgtgtgtgtgtgtgtgtagtgtacacacacacatat
aaataaataaattaaaacaaaacaaaatctgagtcagtttcatcgactttatgaacatcc
catatgagaaaaaaaggctatccattaaaatgatatattttcttgggagccatctgcctt
tagcagaacaaaattgaaattgaaattattttcttcatgaaaattacaaattaccaatga
gaagttatgggtaccattcctggttataggcaattctttctttaactaagctgaaaagta
ataaagttgacttcaatatttaaatctattgattacatatacttccctcatccaatctaa
atcataactttgatgatataaatccttaaaaagtaaatcattaagatatatacttcaaaa
gtaccatttgtgttgctataaactggaatcacttaatcaagaaataagagagaaagaagc
gtgtagcacttcagaacacacgaggggcagcagcctgaagagaagacgtacccacgcacc
aaacattcccagctcacctgcctgcccagattgcatcctgtccactgcacaggatttcct
ttgccttggaactgtcattcataccattttctgtcaagttaagccctcttctctaacatt
ttgaatcagcttctgggttagggattaaggttagtaactattcccaaactccatctttct
ttttcattgcttacatttacatgatttaacttctcaaagcctcactataaggcatacgtc
ttttctgttaaaaattttatatgtatacatatataaaatataataaatatatagtataat
atatatgtttgtatgtacatatatatgtatgtatatttcttttggggggcatggtttctt
tgttagtggtgtggtcttcttttttgtttcttgagatctcacagtgtgaccctgtataat
cctgcaactgctatatagaccagctagccttgaactcagagatccacctgtatctgtctc
ccagtgctggcatgaatgacatgaaacacaccacatctggtctctgcaaaaaaattaatg
tggaataagcaaacatatatctttcttacagaaaaataaatatgtatcagtaattaaaag
caatttccactctggataaaaacactaataaaaatcaaaactacaatctgcacagaataa
aatgtttataaacaactagaactcatcctactaaactaactcttaactaagatgactgga
gttttcagaactgggtacttgtcaacatgggtgatgtgccagtatcaaaactcaggtttt
ttgtaactcctttcttttcccatatcatttacaaactaatgacatcagttcctttgaata
tcttccccacaccagaaactagacccaccaaccgttcattccccttctgctcaaaagctc
tacctccttaacctcagtcactctgctatgcacaagtctatgacttaaatgccaagacag
accagggtgaatggtgaagtctcacaaagaggagagaatttgtaccaatttgactattca
gatttaatttttttaatttgattttcttttcatttctttgatttttgtttttaatgaaac
cctaataatagaatgaaatgttagaaaagcaaaaatcagtattaaagtgtacattgagaa
aaagctgtagtgtcctgactcccaacctctgagtaactaaggactttcaagttgctccta
aagttaacctggatacaaggcacatgttaaagggtgcctctttgggtggcttagcttaca
cagaagcaacaaatgcagaccttagattctagaggatgccacttactgctcttaacatgt
gcccatggaagccacttcccaattcttcttgccaactcagtattatatatgtgtagagga
aatttatcataacatccagagtaactgtgtgaattgaacacaagacaacacacacacaaa
acaaacaaacaagaactagtcatgtctgaagctggaagtaaagtgtcacggctctgactc
ggctcctttcccttaaacactgtttttgcacccactgccaattcgtgaattcagctccgc
aacacttagctccttgtctccagtactcgtccctgcaaagctgctgcttttggcttccct
gccctgcatgccttccccaggctctccactctgctctcaccgtatccgctgccaccaaca
cggctgattcttaaaactcaacgagatgagccatcccatgggagcaagggcactgcttcc
agggcagattcctagaacttgtggcaaaaacaaaaccactggctccttctccactgctgt
cttccgagatggctcgcacactccccactccaaagccattaaagggcgcttaagaacctc
aagcagcatcagccacacctgtgctcttcagtgttggctttcctaaagggcccagcagct
gctccagggcagtttttgccttttctgtttcagtaattccagttgcaccggaatttaact
gtaacctaacaaagtaagtgacaagaatggcatatgaatcgccctcgtagcagacatcta
cagtggttggataaaatgaaaaccatttgcttattctatctcaacaccatcaacagtctg
aagcacgattttacattttacaaaacggattctttacaaaaacaatccttaagaaaagga
aaacttgcatgcaacatgcagaacaagttaagaatgacagtgtgttctcagaaccaatgg
ctctcaaaagaacccagggcttgagagatggctcagcagttaagagcatatactactcta
gcaaaggacctgagttcaacatctggcatccatgcgaggctgcttagtgggatccaacat
tctcttttggccgctgtagacactgcacgtatgcagcgtagactcaactcatacacataa
gtttttttttttttttttaaacaaaataaagatgcctgtatcccattacacagcttttgc
cagtggttactaatatagcgtgaatgtgtgtacgcagatgtgcatgtgtgtgtaggcaca
cctaactctgcacgtgcatctggaggccagagaacaacctgagctgttgtatctcctcag
acacagcaacagcctaccttgtctttaacacggggtctctggctggacctgactagatgg
ccggtaaacaccaggaatcttcttgtcttcttcccagtgctgggagctgcccctggcctg
cccgtgggtaccgggcatcggcgtcaggaacacatgcttgaacaccaagcacgtgacagg
ctgagctatctctgcagctctagtttttacttccccctcctttctgagtgaggcacgtga
tccctgaagcactactccaaaggaggacatgcggtgagatatgtttacaagacaaattac
catgtgactgctgaataagagggtcagcatgtcacctcattccagtcgctgtgataaaac
gcccttaacaaaagcaactttttgttaagagaaagggctgattttggctcacagttgatg
atgggctgtcatgaggggaccctccagtgacattcaggttacattcttgctgcccagcaa
tgcttcccctttctgttcagttcaggacccagcctgtaaaggaatgctacttcctaattt
tagggtgggtctttagagcctaagttatcagttaacctaattgaggagacaggtacaggc
aagggcaaaatctgctttcctgggtggttgtaggtcctgttaatttgacagtattaacac
tttccaaatgctagtggaatcagattttccagacatggtattatgtacacatactggtgt
gtccctgcatctttgtgtgcacatacacgtagaggtcagaggtcggcctctgaagttatt
cctcacacactgtccaccttgttttgtttttgtgacaagtgagctccagggaccttcctt
ctgtctcctgctgggagacttaaatttccaaataggttttaagatcagaagcactagtcg
ggtcttggggtctgggcattgagcacaggtcctcctgcgtggcaagcactttactggttg
ctgtctccagccctacttcttagtaagttagaaaagtcttctgtttatttacaagcacaa
gtagaaatgattttctgacaccttacagtttgaatgcctataggctctcataatagcccc
cctccctcactggttgagtagtgaataaggaggtgtgttaaagtaaattaactgacttga
agatgaatgggaatattgtgccaggtagttttcagtagacacattaggacagacatcacc
cattttacaggcgatatgctctatcagtttctgtatctcattcttacccttgttatccgg
ctcccttagctcttgcctgccatctgagggaatttcaaatgggtttacagaagagctgag
caagcagtttattacagtaaaagttcctccagacagctaggagagggctgacaagaggga
tggcccacagctctcaggctatgaggtttgcctctccccccacccccaccccccatgtcc
tctacttatgtccccttcctcccaccccttttcctctcccctccttgagccagggttcta
taaaacaggttgtttttatcttctcccagtgtgtgcactcattgctaagttgcttgtgat
ctggtgtcctgatgataggatcacatgactgtttgtcagctctgatctcctgatgccaca
gagctgagattaaacaatgcagctttaccccctgaccccttccaccactgttgactgtga
cttctacccatgcagtaacaacttggaataaaagtaactcagataacattgacctgatga
agctgtgggggagtccttgtcagaatgacagatctcatcatcgtttgcattttgctgtca
tcttttcttgagcagtggctttaaagtggtgatcaggtgattctattcctaggcagctta
gctcaagcaaatttaactttctctgaagcagtataaattccaccaaagacacaaagaatt
cttataaagctccaaggtaaaattccaggggacagtgaagcttcaacaacaagtgaattg
aagaaaaatccttgttctagcatgcctctcatttgccttctaaaaagaaacagaagaatg
gggaagagtctactaaaaactgaagaagtatacatatgtaaagctcagagaatgtcagtc
aggataaatacaagtaaactcactctcagactcagcacacagtaactgcagatcagtcag
cctaaagaaactatttaaactacaactggagagcagaagtaatttacctaaaagaactag
gtatttcactgcattgtgagtagaaatagtggaagcctgatgatggtaagttcagcttgc
tgagaagggtggccaaatgaagacctgctgtggaggcagggaggctatcttagttagggt
ttctattcctgcacaaacatcatgaccaagaagcaagttggggaggaaagggcttattcg
gcttacacttccatactcctgttcattaccaaggaggtcaggactcgaactcaagcaggt
cagaaagcaggagctgatgcagaggccatggagggatgttctttactggcttgattcccc
tggcttgctcagcctgctctcttaaagaacaaagactaccagcccagagatggtctcacc
cacaaggggcctttcccccttgatcactaattgagaaaaagccttacagctggatttcat
ggaggcatttccccaactgaagctcctttctctgtgataactccagctgtgtcaggttga
cacaaaactagccagtacagaggctgatgtgtagattccgtctgtattggcttttacccc
tcatgtgactgctgcttactataccagaggtgtcagaggaggagtcttcatgctgggtta
cctgagccaaaggatggcagctcctgcacagactacagtgtctctggaaagcactgctct
ttacagtctgcctgccagtctagttttgcagcatttggtgtttggttaccacgggtttgt
gtaggatttgtgtcttagcagaaaaaaagcaaagggaagctgtgggagtcaggcacagca
acccccagattacacggttccttacagtgttcttcccttatacctccattgtaagagggc
tgtctgtaaagtgggagttgctgcttctactgatagaccactagtgtctggagccaagta
aatgcaagtctaatgtgtgctgagaccacactagaaggctccctgattttcaactctata
ttttcataggagaaatctcttagaactttttaatcaaaggcaaacaggatgatggagttt
cccatagccgttattgattatcttaatttacaaaactgaaatctgaaatgccccagaatc
caaaattgttgactgtcatgttggcatacagaaagtttctgatttgggaacagttggact
tcttgagattttgaaataaggaatatgcagcatacagcatgttaaatgctgtgtgccaaa
atatcggtcagcactgtatatgtacactcttatttaatctttaatacttctataagacaa
agtacaatcatttgaaatcctctctgcagattagacagttgaggttttaagacactggat
aacttgtcccgttttgtaggtagatgctggcacaacttgtgtttgtagctcctcactacc
aatccgggcctcttatccaccaagattatagcttgggtgttatgtagctgtgtatttccc
catccaatgctttaataacatccagcaaggtttatatttttcatgtcctttgttcctatt
tatttttactttcaacataaaacagctctgctggaattggaatggcctggctgtggccac
agtattgtataacttccacagccttgggagtgaaattcaggcaaatatgttgcagcaggt
ggtcagtgcttaaaattatttcacttgtgatacagcgtgtgtgtcagaggtttactctaa
cagtaacattacgctttaactgtacagcacctggcataatgtaaggttcagttggagata
attgttattttttttagttgtgtggggaattagaaggcagcaaaataagaaagttggatg
tgtttgtgttagaattcaaaattttattttcattattttgccttaaaagtaattttagat
gtcaaagtacttataacttatgtaatacagtgctttttaaaatttatctgcagctttaaa
aactctttgtaactggtaaggaagcatgcattagttactgttctgctgctatgataaagc
accacgatcaaggcaactgatggaaggaagggttcatttagacttatgttctacaaggta
aggatatagcagcagcagcagcagcagcagcggagagttcacatctgaaccagaagtggg
aagcagaaagagggaatgcctgagtttttgaactctgcaagttcagttgcagtgactttc
tccagcaaggctactgctccccaagcctcctaaaacagggccaccggctggaagccaagt
attcacatgccatagactagaaggggacatctgactctcaccactaccaagtgcagtaag
aatgaatgcatgagtttggggtgtgcacaattacaaattaccaaaactcattagtcctga
tacaagttctaaggaagagagttgtgtcttagttagggttttactgctgcgaacagacac
catgaccaaggcaagacttataaagaacagtatttaattgggctggcttacaggtccaga
ggtttagtccatcatcatcaaggtgggaacatgacaggcatggtgcaggcagagctaaga
gttctacatctttatctgaaggctgctagtgcaagactgactttgaaagatctgcatgcc
tttcttctaaacactgggattaaaggtgtggtccaccaagcctggatttaagtttttctt
tacctagaacttgctctgttctaggctggccttcaactcatatctatttgtctctgcctc
ctgggattaaaggcttgtactaccacacctggacctaaatttagatgggtggaatcttgc
cccaaggtcacttctcccttaattcaaattaatctccttgagcacaggattcagctccat
ttcacttcctggtgtccctttagtactcaaactttatattttgtatttttcctttctcag
cttgctacatttgttcaaatgttcttcatcagacttaaccagagaacaaagtctgttggg
ctttgttgggacttcctttgtcaatgcaattattataaatctctttagtcttagtcttag
gcagtcttttcagacaatggccacgttcaccaaaacaacacaaaacagtctctcaccaca
tattaaagttcttctccactgaaacctcttgggctagcatgcctaaacagttcaaatgac
cctcagcaccagtcttccatattttactaggttggctcattaagccccacttaaagcatt
ccttggcttcccaaacccaaagtctcaaatccacattcttccaaacaaaagcatcgtcaa
gcctatcacagcagtgccccaatccctggtaccaacttctgtcttagggttttactgctg
taaacagacaccatggccaaggcacctctcagaaggacaacatttaactggggctcactt
acaggttcagagtttcagtccattatcaaggcaggaacatggcagcatccaggctggtgt
ggtgcaggagaagctgagagttctacatattcatctgaaggctgctagcagaatactgac
ttccaggcagctaggagtagggacttaaagcccacacccacagtgacacacctactgcaa
ccaggtcacacctattccaacaaggctacacctccaaacagtgccactccctgggccaag
catatataaaccatcacaagttgtcactgtcttaatcttaggagggagcttttccacaaa
aggagtagtcacagatggtactatttgctgttgtaagttatgtcttgtttgtggtggaag
acagatttatacgagtgacttagaaataacctgcaccagctaagcctctggaccagcagc
cctaagaacttgtttcctgttccactgcctgggaatacttatctcagagtctagattagg
tgaatgtgccccctagtgttagagtgggcactctgactgcttgctggttaaatggaagca
tctttacccatttaccaactggaacataaaactggctttgtacctgctaatattttctgt
attcttagtatcttggttggtagattaaggaaatggatctatagtattttctgttccata
actccttaattgctagtgtaggtaatggatagtattttctgtcttttctttttcttagtt
tgaattagtgcttggatttttgcctagacaaaaggagtaacttggaatgaagaaaatgga
ctcaaacttaaagagctgcacactaatcaatccttcgaacgcacatggaaaccagagacc
acccctactgtcctcttagttacacaagtggctgaggttagtgataaaatcctctgcagc
ctccactgaattcaagctgtgaccaacacagcattgtctgtatggaaactggtgcctcca
agttggagagtgcttacaagtgctgccttgttaggttggtgcagaagtattgttctgtaa
cctgaacactctgaaaatttcttgtgtgaaaaatacagattattacaaacaggatctatt
ttaatgattcattaagacactgttattttatctctacacaaatggaactctttgtaagag
aatcacagtatttcttaccatttcttacctcataaaaccagtcagtacaactgaccctgt
gtcaacttgacacatacatccatcactagtaagctataatctttcctttcttaatcatct
ccagatctataactttaaactccctcattttttacaaattcaaacacattaaaattcccg
tccctttaaaatagccagtttcttaaaatccaaagtcttttaagacaaagtctgaattgt
ggattcaaagtcttaactgtagactccagtaaaatacttttttacttcaagaggaaaaaa
aaaatcagggcacagtcacagtcaaatcaaaatcaaactccaactgtccagtgtctagga
tctactcataatcttctggacaccttcatagggcttgggtcacttcttcagttttgccct
ttgcagtacacacagcttgtcttctaggctctggttggctccattccactgctgctgttc
ttggtagtcatcccatggtactggcatctccaaaactcaagcctcaacttctgtgaaggc
cccttcagttctgggccttcaactgccactgaggcggcaccctcaccaatggccttttct
ggccgctcccagtgccaaacctcagctgctctccatgacccctttatatctttaaaactt
gtatcgtctgggtgactcatatattcctatgtccggctgccaacatgaggtacaaccttt
gttgcctctggagcacagcttctctgtgctctcagaaaacacttctcagaagactttatt
tcattaatactggtctttctttaatcggcactaatttcttaggtccagctaaccagcaac
aaatgttctagggatgtaaaggtttcactttaagtagttctggtgtcttgttagtcacag
ctacagctggttctttagccccagctggccagaaccagggtctaaatcacagtaacaaat
ggcccggatagtctttaatcttccctctgaaacttcacaagacaggcctccatctttatt
gctctcaacattcttttttttttttttaatattttttaaagatttatttatttactatat
gtaagtacactgtagctgtcttcagacacaccagaagagagcatcagatctcagtatggg
tggttgtgagccaccatgtggttgctgggatttgaactcaggacctccggaagagcagtc
agtgctcttaaccactgagccatttctccatccctcccacccccgcccctctacattctt
atcttacaggttccttcagaacatcccactgaggtgtcaacactcaatggttcttctagc
ccaaagttcttacatcctcccacaatccttcctaaaacatgtcaggtctcttacagcaat
aaccttctgtattagtcagggttccctagagacccagaacttaaggatagtctctatatt
gttagggaatttgttgatgacttatagtctgtagtccaactctccaacaattgtcagcag
tagctgtgaatgggaagtccagggatctagcagttgctcagtcccacaaggcaatcaggc
gaagaagagagagtaaatcttccttcttccaatgtccttatgttggtctctagcagaagg
tgtggcccaaattaaaggtgtgtggctcacgcctggatctgggacttgctttgtcactat
gcctcaagagctccatgccaagatccaggtcagaaacttgtatctcccagattaggatca
caggtgagtcttccaattctggattgtagttcaatccagatatagttaagttgactacca
gaaacagccactacaatcaccccttgtcaacttgatacagataacatctcatgtccacat
gaaacaataacaaagccaagaatacacctaacatgatatacttactatccgtcgtacaat
tgcaaacacatttgtaaatttacaatggggcaatgtcccttgagaacattcttttagtgt
ctcaacttaaataacaattgatgttaaagaaattggaagaaaaacaaatgtattctaaac
aaaataagacagaagcatttacattacttgataatcctcatttctgcaactggttacatg
gccttagctggtatttataactaccttcctctactacccattctgtatttccttcaccct
tggcaaacacctcagcaggtcttggctcttttcctggaggattgacccataccttcattc
ctgatgggtctgcatcctttgtcaccttgcttggattaggctgttgtagtttcccattga
ctttaatcacagggcatggtagtactaagagacgccctaaggggtctcctacattccaca
cataatcctgcttacctccattgtggagaggcaatccaatttccccatggtaatctggat
ctatcacccctcctaacactgttattcctttcttagcctgttggtttaagggcattagaa
gcccaaaatgaccagggggaagtctgagctttcagttcaatggaatgtttgttgtggttc
ctggtatgagcactccctgctctggatcccaaacttctaggccagcagaacctagagttg
tggggacaggatgcaaaacatttcctataggttcactgggagtgataatgagtggaacta
ttcccttttccaccccttgattcctggacccatggatcctgactatgggggaaactctac
catatattggatgctgattcaaagcatatactgccttctgaagaactctgccccagcctt
ccatgctgctgccacctaattggtgctgtaactgtgtcttcaaaaggccatttcatcttt
ctgtcagaccagctcctttaggatgataggaaacatggtaagagctgtagcactttgctg
gctgtgaaatgagttccttggtcagaagcaatactgtgtggtataccatgacgatggata
aggcattctgtgagtctgtggatggtggttttggcagaagcattacgtgcaggaaaggca
aatccataaccagaaaaagaatctattccagtaagaacaaaacattgtcctttccatgaa
gtaagtggtccaatgtagtcaacctgccaccaggttgctggctggtcacctcgaggaatg
gtgccatatctggggctcagtgttggtttctgctgttggtagatctggcattcagtagta
gctgtaaccaggtcagccttggtgagtggaagtctgtgttgctgagcccaagcataacct
ccatctcgaccaccatggccactttgttcaagtggtcattaaacaacgacatggatggct
ggggaaagaggctgactgtccacagaacaggtcatcttatccacttgattattgaactcc
tcctcagttgaagtcaccttttggtgagcatttacatgggacacaaatatcttcacatcc
tttgcccatttggagagatttatccacatacttcttccccagatgtctttctcaacaatt
ttccagatctttccaggtccttgaccatccagtcaatccattggctatagcccactatgc
tggtactaacttgtcttagggcttttaatgctgtgatgaaacatcttgacaaaagcaatt
ggggagaaaagagtttatttgacctacactttccaatcatagccggtcattgaaggaagg
caggacaggaactgaagcagagcaggaacttgaaggcaggagctgatacagaggccatgg
agagatgctacttactggcttgcttcccatggcttggtcagcctgctttttcatagaacc
accagcccagggatggcaccacgtacaatgggctgggccctcccccgttgatcactaact
gagaaaatgccctacagctagatatcatggaggtatttccgaatctgaggctgcctcctt
tcggatgactctaactcttgtcaagtcgacgcacagtacagcgagtaaggagagaaatcc
agacaggagtttttaaaatgggaagtaggtgaggtaagagcagcctggagctagccactt
gttgcttcatatttgacaaagattactcaggagatgagcagagaggtaggctctatagag
gatacacttctcggttccaagatgcaaactatacgaagacagttttctttttttgtttca
caaaaataatgttaaatgattagctaacaattaaaatgatagctaagttgggagtcccat
taaagaaacagactcctcactggttgctacaaataatgtgttttccatactgcagtgaaa
gatttattttgaagttcagaacgttcctgcaaaatgatagtgaggcacagtggaaagaaa
atctaaaaggatttgaatccatagtggttactttctagttccttatgatgctgctgagca
aggctgtctctctctctctcactctctcactggctcttagaggtttcaaatgcttcacct
atggctgttgcttgtatgaccctctcatattctgtcactagtagtgagctgtgggcacac
acctgcattgtaggggctgtgcatttgagaaagcctgtcactttgcccattgaattggga
aaccatgagtctttattctgattccctgatgagatttttatctgatggtactgtagggga
atccttaccactgaggacacgggctccaaggccaacaccgtccttgaatttaacagcaga
cagtaggagagtcaaaaacagatagagaagttttgtattatcttcagttttacagactcc
ccccacccaatacttaatatagatttaaaccaaagtactctaactaaatactgaaagcac
agttaagctctgaacaatacatacacttatatacccaacacatcagtcaagatagagatt
atcaccctagaaaattctttaaggatatattttatgtgtatgtgtgtattagtatctgag
gagaagagggggttggatcctctggagctggagttacagctagtgaactgctcaatgtgg
gtgctgggaactgaattctggttctctggaagagcagcaaatgctcttaactccactgag
tcagtctttccaggccttactcccaaattcttttaagccccatttcagtcactatcacac
ttagaaaaactctgtactgtcttacatcattgtaaatagtttaaaattaaaaaaaatatt
gtcagtggaattttacaatatttcttttgcatctggatatgcatgcatttacattcctgt
gcatgcacagaggccatcctacccttttccccccaaactcccttgacacatgctctctca
cccaatttgaagctggactagaggccagaaaacccaagtaaactctttgtctctgccccc
tcccacaatgaacttggtgaatagattaggctggctggcctgcaaaccccaggcatccat
ctgtcttggctgtcatttgtgggggaaagttaagcatgtaaatgtttctaatctgccacc
aaggtattggcatttcatgttcttgcagtgattcaaagggtagacaagagtgatagagat
acatggtagaaatgactagtgatactacttaaactgagttcaggtgtgtgacttgtctgc
tttattaagcagggatagggatctgagagtgattgtggcactcagaaggacacatttaat
tctagaggctttgagcccatgatcatcatgccaaggaacatggtggcaagaaggtactgc
agtggatcagatcagtagctgagagcttacatgtgctctaggagcatgaggcagataggg
agactgagaagtgggagtgctgtggggcccttttccaaacgaggtcacacctcccaaccc
ttccttaacagttccatcaactggggaccaagtactcaggtccaagggactattgtcatt
cacaccaccacaagcactaaggacttactttgccctggtcatttggtagaaagttaccaa
cgtgatgtactgtctcatccaaacctgccctttgttccttctttccctcttctcccttcg
cccctttcttttccacacccttccattttgttttcatttgtgtgttgggggtgggtgggt
ttggttgtggtttgtgtgtgtatgcatttgtatgtggggacagaaggcagttttggggtt
tcattctaccctgttcttgtcacacagtctcttactgagacttggggctaggctgtgcag
aacttacagacaagcctgcctctgcctcacaacctcacaagcactggccttacaagtgca
tgccaccatgcccagcttcctacatgagttctgggtaccaaactcagtttgactggcaag
ttctttaccagctgagctatgtgtctccgatccagtgtacattttgtatgaataggacat
tttcatgcataatgacagtatagatataaaagcaaagatgttggcatcaatatggcattt
cccagacctcctttcagttaaccagttatctggttaatgtgtcttgtgagagattgactg
caagactttgtgttgcatttgttaggcttctagaatagttcattgatctcatgtgctact
ttcatccctttcaccacttggaggtgcttctgattctgtacagtagtaactcttgcctca
tgttcccttagtagtggttctagttgtgccttactagtggttcagtatcagaagtgccag
agaaatgatgctgcctttgtcttgctgcttagggattaggtccatcagcagggatgcact
tttatttattgtcagtctgtctacatgagaataaaaacattcacatctttaaaacatctg
gctagttcaagattcaagtgcttgccttgcaaatctgacagtaagtttgtgcctcagaat
ccatagaagggtaggaaggcactgcttccacaaagttgtcctctgcccaccacgtatcat
ttgtagcatgcacatgcccacatgcatattatacccagacacgcagtaacaacaggaaga
acacctgcatggcaatggttatccacagctcatggtaagtaagttagcgtggtccctgag
ttcctgacagtcagtgtctgatcagaagctgctctgtctcccaccctgtgttttgggtgc
tcattgaaacctctttttctctattgcactcaccccacaaacagtatcagctcttactaa
agttattaaagcaagtaatagagattccattttttctttttaaacattatacattgtctt
tattacatcattagatcttatcatagatttctgctggcctggaatgtgccagaataattc
ttacacacttcttattaagaataaaaagtcatatttaaatcatctttttggggggaggtt
gcaactccctggtaattaattcagtgtttggtctttgaaatcaatcccttatcttttcat
aaaattttcaaactcaagaaatttaataaaataggtttagctagtttgagccaaaacgaa
gcttaatttttcagaatctcggcttagatgacttaaattaaataaaggccataagttgat
tgattaattttatttgtagcgtgacagtttattgatagtattgcatagttctgttactta
gagtggtgccccagggaccaggaacagataaccagactcgatgtgattataggactgagc
aaacagtgctgcctgcccacggagcctgtgcagtgcggctcttacatggctcctcctctc
ccaatctctgccttatacttttgagcctcgtgaatagaaactatttcacctgcccttatt
tcctgccacacaaataatgtgggtcacatggccaggtgggttagttggaaggtctcaccc
tcaagtgacaaggccaggccgagcttgtttgtcactgctgctgagttagaaaggctcctg
tggtatgcaatttgaaaacttggcataatgcttatgcttccatatcaagtcaggcttcta
tttggccaggtcgggaataggctttttctcagttactctgtgacaggagatggagcacct
gcacttgctggtccccttttttttgctctcaagccgtagcagtggtagtatcatccgtgg
cctcttagaatgcattgcttgtcaaaagcttatattcccattgtatccaagagcctcagc
tgatggaagaaacagctgccatattcttgatgctgtacagagacagacaataaatatgtt
acaggctagcttctgcacccttcatctttcaagtctagagcctagcccataggatggcat
ctatatgtaaggcaagcctttcttctttagttacatatttctggatgcaaccacagactt
acctggagagatacatactttaatagtgatcctaaatccagtcaagttgacagtgatgat
gattaattatctcactcttctggaatgcccattttatgtccgtgttaatatgtttcttag
tcagtgttctgttgctatgaagatacaccatgatgatggcaactcttaaaggagaacatt
tcattgaggattgcctacagttcagagggttagtctattatcatggggaagtatggcagt
ggatagtagacagagtactatatgtagctttagctgagagttctatatctggattctcag
gtatcagggacagggacgaggatggacacacacacacatacacacacacacacacggggt
ggggagagggagggtagacagacagacagacagtgatcctgacttaggcttctgaaacct
gaaagcccacccccccaaacccctacctcttccagtgcacacttcctccaacaaggccat
acctgctccagcaaggccacacctcctaatccttctcaagtagtgctactctgggatgac
caagcattcaaatataggtgcctgtaggggccattcttattcagaccactacagctatgt
atcactgaggatcttttcagctctttttgtggttcttgattttttaccttaaagttttta
cttcctgccatgggagataaagaatcaaaattactaaaaagcaaaatgagcaataataat
ttgaaataaaagtttttagttatagtattagtagtctgtactaaagatgacaagatttat
ttttttgtctattttgttttgagacagtatttattatgaagactaggctggtcttaaatt
cacaagtttctgagttctaggattacaggtatgagctattaaactatttgctaaagagga
aaaatatttcatgattttatgctttataaaaaactaaatataatttataaataaatttca
gagtttagaaagcatttatctttgtggtgctgggatgacatagtaagaaaggacagtgcc
tttctgatatggcctcacatctagtgggggaaggccatacatataaatgtgggcttgtgg
atccataggcacttcttatgcagcaaggcttattctagtgcttctatttatttagtatta
tgggaagaggcagggcaaggggagaggggagcttagtgcaggggcttttactgattttag
tggactagtgggagagtgtacgtatgtttgtgcaggtttgtatgctatgcagagtcctga
agttgatgctgggtgtcttcctctattgctctacaacgtatattttgcaacagggtctct
cagaaaactgggtgctctagattggcaggctaacaaataccattgctcccctgcccagct
ctgggttcatagacttggattggaatgctgtagttttacatgggtgctggcgatctgacc
tgagatccttatacttgtgaagcaaatactttacccactgagccatattatgagcttacc
tgtgaccttttggcagttatttgtggacatgtacaggactcccccctcccaaaaaaactg
gacccaattggcatgcatgtgttaataagcaatgctttaccttcttgtttcagctccaca
tcataaacaagtgtccttttcatgtctatactgaaatacttgcccatatttgttttcttt
ttgttgttgattatgttgtttaaaatgatccccagtcatacaaccaaattgccctctaat
atttgtaagttccaaaatgctttgatactgccatgtgaaaaaaatactgcagtttagcta
agtctcgtcccgacctgagttgtattatttgggcatgagttcagtgttgagtcaatactg
tacattgaataagatgccttttggctggtaggtcaacagtgttgtggtctagatgcattt
ttcttcaagagcagtggcttgggatttactaattcagtgggtaaaccaatttcaccaaac
attctggtcatgactaagagaactaactacatttaactaatggtggtgacctatagagat
tttaggtattgtgaatgttttattatccccactccgtctgaaactactgtttcagttacg
tttaatatttccttgtagccttgcatgtgaaatcctattttactgtgagttctggtcaga
aaggcctgacacaagcagaaacatgtcttagatttgctgttgtttgtttttttttagggg
tctttgagacagggtttctctgtgtagccttagctgtcctagaatttagctttgtagacc
aggttggcctcacactcaagagatctacctgctcctgcctcccaagtactaggattaaag
gcgtgtaccacgactgcctggctagaatttgttttataatttattcttggtgagacttgg
gattaggtaaagaaacaattgtaaatacttctctccagaatattaatgtggatttgaagg
aggttcttgttttagaataataatgtgtgaacactgcattgattctgatgttagaacagg
caatataagtgttagcacatactactgtcagggttttatcatgtgcttcctggagactaa
ggcacaagaagaacatgcagttacaagttcttaaacctgggctgtttttgaaggcagcat
ccttagcaagtgtaattcttttgagcctcggttgtctagtgtttagttagaaatatttcc
agtttgaaagttgtgcggttttgtcagtatcatttacattctcagcatgtccagttgtgg
aagaaagagttcagtattttgaaaattgtagtgttaaaaatacattagctgtcagatttc
atttttctaaagtctagctaagtctcattagatattggtagaaacataagttttgtacct
gataaatattgacatcatgtacttctgctatgaaatagtagagcatttaaaaatgttaaa
gttgaaaatttcaattaaaaaacaatagatactatatgtttgtgttttatataagtgttc
tgattcatacaaatatttctctaaagtgttcagaattagtttgtaaagcaaaatgtgtat
tcttgggagattattaacctttttgcatcaagtttcaagttctaggtgcttttttaaaaa
atgtatgtaatgtgttgaaattattttaggaaaacttgaacatggtcaaatatcagcaaa
gaacgttctctgtaatacacagactgaacgctggtatagctgtcttgtatgaggctatgt
gccagcctggcaaatacagaagtggatgcccacagtcatctataggatggatatacacag
ggcccccaatggagaagctagagaaagcacccgaggagctgaaggactctgcaaccctat
aggtggaacaacaatatgaactaaccagtacccccagagcttgtgtctttagctgcatat
atagcagaagatggcctagttggccatcactgggaagagaggccccttggtattgcaaac
tttatatcccccagtacaggggaccgccagggccaagaagtgggagtgggtgggtagggg
agcagggcgggggggagtatagggaactttcgggatagcatttgaaatttgaaatgtata
taaagaaaatgtctaattaaaaaatacacagactgaaaaatgtaacatgaaatattagac
catatgctagcacacagaaaaaaacattttcagtatgtaagttttgagaaataagttgaa
agcctccataaatacccaatcttcataatgcttttaaaatgcaacagcatgtcttggcta
caaattctagctcatggactaggttcttgtaaattcattaactagttaattaatttatgt
atgtgggtatttttgtttctatatatgtctgtatatacctgacctgaagaaggtgtcaga
tcctctgtaactggagttacagatggttgtgaacctccatacaggtgctaggaattgaac
ctgtgtcctttggaagaacagccagtgctcttaactgctgagccatctctccagccccac
tcatgggctcttgtatcttgtatactaaacatctggattttattctcattctccccaccc
cctttccatcagtgagcctcttctgtataaagttccctttcctattttcagatccttagg
ataggctaccacaagtggagttactgggttagagagtatggacttttgtgtgtttttacc
tacaatatttagatttgttgttaattgatttatattcattgtagaagataaaaattacag
cgattgtgcttcttatctgaaggtaaggttttatggttcaggaagcttgggaaattgcca
ggataataaagctccttgaaggggacttatgccaacatccctgaaccttgctggtcactt
tataggtggctttgttgttgttgtttgtatatgtttaatagttagtgaatgatttatata
ggaattattttattggctaagttatatcttattgatccaagcaactaaaccaccaagctt
ttagagatttatgagaaaaggtagttaaaagtctttagtatgattttttttgaaaaaaat
tataacttacttaatttaaaacttatttattactaacgagcgcgtgaatctttaattata
tacttagactataggcaaatgacacgaaaatagacagtaacaaagattgtacaaatcttg
tttatcccccttttcctcctacagaatgtgaaaacctttgcatcttctgatagtctagcc
aaggtccaagaagtagcaagctggcttttggaaatgaatcaggaactgctctctgtgggc
agcaaaagacgacgaactggaggctctctgagagggaatgcttcctcaagccaggttgat
gagggacagatgaatcgcgtggttgaggaggatccacagcagcaagcgagacatcaagag
gaggagcacactgcgcggaatggtgaacttgtgggtgcaaaccctaggcctggagaccag
aacgatacccagcaaggacaagtggaggaaaataataaccgctttatttcagtagatgag
gactcttcgggaaatcaggaagagcaagaggaagatgaagagcatgctggggaacaggag
gaggaagaggaggaagaggaagaggaggaggagatggaccaggagagtgatgattttgat
ccgtctgatgacagtagcagagaagatgaacatacgcacaatagcaatgtcacaaactgc
agtagtgtctcggacctgcccgctcaccagctctcctctccattctatacaaagacaaca
aaagtgagtacctttagtccccttccagctggtatgctacctcgctagttcagttatagt
gaagtcatccttgcaatcttcacatgcagaacaactttagtaagtcagttttagtttgtt
aaaggctttagtgttagaaatacagatactaataagaagtcaacttaattgcaaaactag
ggggtgcggattgatgttgtttaccaccaaaaaaaaaaaaatcaccagcttcacctttgc
tgccagagtggtttgggtgagtgtgcagggttggtgtgaaatatgatgtgtgtaaattaa
gtaatgaagaatcttatttgcagagtagctaatgtagtcactgtctgtcgtttcaccagc
gttaactctaatgggtaagttagtggaagtcacagcaaatagttctgcgttgttgttcag
aagttaaccgtggctcatggatgtctaagtagactagctttcagttaacctgttctaggg
attgcatggagtgacaggtcacttgttctacacacataaaaacaaacagtccacatcaga
catccctttactcatttgctgatttttaaatttaacaatgaatcattggatttagtctgt
aaatacctgtaaatttaacagtatctctttcaatatggtttgactttaactatagtatct
aatacagttggcttaaaattcaatttgctgaatcagttttgtttttaggttgtaaaacaa
actgtctcagagaacgtttgtacctctaatctccagagcctggtcacgttgttttattta
cttgctggcatggcttaggaaggaagtggcatcaggtcatggctctctcccatttaactt
tctcaaaatggagggttttggtctgggctgattcttagtgttttgccgttctttttattt
ccgtcatagcagtttagaaaatcctctagcactctggagggcagggcaggaagattatga
attcaaggcaagacattctctcaattgcttaaaagacgttttaaaagttacctgtagctg
tattaagacttcacatatcgaaatctattaataagaagaaatttatactaattcagtatt
ttattttggaggcctttacatctttgatcattataaaacaacagaattgtacagtatcct
aggctgcagtttcatttcagtccgcaacgtggggccttagagaagagtagggctgagatg
ggaatcctactggagaggcaggtgtggcagcctcatcacttaaggcaggaagattgttgt
agatttcaggctggcctgggctgtatagtgggtgtgagtctgggatggactgttcagaag
acttttatctttgaaaggaagaaaacgtcttccacgttctcagtttgctttaaaaatgtc
cctgtagaagtctccagcaatcctgaccgcagctccacttagtgtgtcattctgcagcat
ttctgcattaatgagaaaaggcgtgtgttctctgttgcattggtttactctacagagcac
aatggagactgtccagactaagggcagtttcctgcccttggtcattgagctttccttttt
tgtgagtgtagagaagacggacagcagttttgacctaactgtgaagtgggcacccttaag
tgctgagtataacagtggccagtggttcaaggcagacttgtactagttcttaaaatttct
gaactcttgacaggttttttgaggtgctttctgtccaggtgactgacagacccatttgga
ctaactcttagttatagggaacagtctctgctctttataagaaagccatgtttcctcttg
gggccattaagttttgttcttaagggaatatgccataagcagtatagagtatattagata
ctagataaacagatcgggaattctaatattaagtgtcaaggctaattgtagtcaataatt
gctgcttattaaatacaaactgtttacacgttcctgaatagataagtaaacaggttatag
ggagcccaatggttcactccacaccacactttctttctcatgtttagatttttttctatt
tttcttttcttgagaatagtttttttttaaatacaatatattctgattatgcttcccccc
ccaacttctccaagatccttctcacctccttcctgtctgtacaccccaccctttctgtca
tgtaaagaaaaataatagaccatgataaaacaaatccaaatgaataagaatagacataac
tggcaaacaaacagaagcaaaagagccaaagcatgagtaacacatacagatgtagagaca
taaacgcttatacacataggagtccataaaaacaaagacctggaagccatagtatatatg
catagggtctgtagggttaaaaagatgaagctttgacaaaacattgtgagacagagaacc
tccatggatgccatgtagtttgtttgtgttgatcatctactgctgggcactggaccaacc
cttaagagtggtttgtttcctcagtgagactctgttggaacaaagtaattatcaattgca
aatagcttctaggtttgcaatggggctctgtctacttttcctctcagtgctgggacccca
tctggcacagacctgtgcatgttctgagtaacaagactttctaagaaccctttctctttc
cgtgtgctatgctgccccctatggtaggatgtaacacacagtacagcagagtttccaagt
ttgataatgaaccccctgagaactcagctagctgcttttcttgtggtgttaacctgactt
tattgaagatatgaggccacaagacctaagttgtaagaggaaggaaggatcaggaaaaaa
ggaattggaggctctggagtctattctatgctagctgtttttctctgtgtgacaagtctt
caccaagttctagagtcagtttgctttgccagttatgagtgtttgtctttgcaggtttaa
ctgtgccagcttactatatctttttttttccctaaattattctctcaagagattctcaga
tcatattgaatctttggcatgtgataatttaaaatacagattattaacgtttgcttctgc
tttatctttatcagctcactttcagctaacaggaaaagcaaatggagctacagaggtcag
gtctttatcctcttgatttttttcgtgaaaacaagcatgtttgagaagtcatttctgtag
taactccattctttatctctaattattagattcttcctagtgatggtttttatccatttg
cttctaggaagaactccatatacacaggcatacacatgaatacatatgcaaagtagatag
agaatacattgtgtctttgagctctaaacagtagaggatgagcagggtctagttgcaagt
gcctatagtcatggcgaccagagagaagggctttagaccataaacttaaagtaaacaggt
ttgtgggggctctactggagctggaactaccagaaaactctatataataatgtgaccaac
agctgaaacttaaagggaagctaggttatactctagagaatgatgttctcctcagaaagt
ccatggcttagttctgtccataactctaaactcccaagttgtattaggtctaggtatgtc
cagagagcagttgtcttatataacatttgctattatgcacagccctgtgagtggctgtgt
cctttccttgtaaatgagaagaaatggaaacatggtgaggtctgcttaatttgattcaga
atctgggggagatgatggcagggaaaaggcaggctgtgaaggatgaggtaggttccctaa
acacggggaactgtagcttgtggtgcctgaacgtggactcttgactctttcactgactat
acgaccatgtgtgatagatagccttattgaacttttctttatctgtacaatgattacctt
tttttagaagtttttcaaggattaagtaaaatcaaatatatgaagtaggtagcatgatgc
ctgagaaacgaggctagtgtgttcccataggccggtgacctggctttgtatctctccctt
aaaatacaattcattctctataacacgtatatctttttgtgattttcagtgttcagtcat
tatgtcactgctaacatacaaaagatatggagaaataagcagaaaataggttaatacttt
aacaaaatgcatgcaagtagttgaacttaataaactgggcttcagtaaaaagaatataag
ctctaatcaaagtgttgccagatcttgtcagtcatcaggctataagctattacttggagt
agaatttgtcagtgcatctgtactttttccaaactttaactgtaaagtttctgcttacat
gctctgtaatccttgcatttgattgataaagtcaatgtcaaattgtggtaaatttctaat
taatttctcttaatacagtttttaaaatatttaacattgtttgaaactatatttgctcaa
tatatatatgtgaatcagattatttttaatctatattccattcactgaaagtagtgaaaa
gaatatcttaattaagagctttctctgtaaactaaagatattttccctatagaacatgtt
accttagcacttgagaagcagaggcaggcatttctctaggccagccatagctacttgatg
agaccgtgtctcaaaaacaaaaacagaatggggaattttaaaacttgagtacaaatgata
tcattgagtacaaatgataaacttctaaaatatgtattagtaccaagcagagttattcca
agatgtttttgaggcagatcgctttatatgtataattacaagggcagtctttttaatgtt
attttgttggtggtaaggaagatggtatgtagaataagcatgcagtcttcaggatgaatt
tcattttagttttgatatataacttcagtattcttaagtatcatagcaggcatttgttag
tatcagaaaatatagatgtaactgctaggaaccacttattttacctaggcttttgtgtaa
cttttctaatgcataatagagggttagagaaaaaggttttcttcccaagaactggcacat
gcccttaaaggagggagagaagtgagactagaaagtgtcactcacttgtgtagtgggaag
ggtgggtgctttgctgtctggccttgatcagattccacatctcagacacacttggagctc
tggcaaattctagctaggtcagctgactccattgctttctgagtttggctagcatggggc
tctatttcatttatgtttgtgcgtacttcatgcatgtgggtactttagaacttagaacag
gtactagatcctctgaagctggagttaagacagcccttggtgcctggtggggattctggg
aatagtactcaggatctttggcagaccagaaggtgctttaactcctgagccatttcttaa
gctgaaaagttttttagtatgtagtttcctgaacaaattctttagtgtggccctgaaagc
ttctggctctccatgtgtctgtcacatttctattgttgtaaagagatgccataaccaagg
caacttatgaatgaaagcattcagttggggacttgcttacatcatggtagagagtgtggt
ggcaggcagacagacaggcatagcactgagcaagtagctgagagctcacatctgaccctc
aagcacaaaagactaggtctggtggtgtgtgcattgaaccctcaccccacccccacatcc
cacgattaatttctctaacaagggcacacctcctaatctttctcaaacagttttataaaa
caggaccaaacatacaaatatatgaactagtggggctcattctcgttccaagtattatac
catgaaacataaataactttcattgagggagggtggaggggacacagtttgagaatcctg
tttttaaaataattttgaggtgcccaatgctttagcaggaaggcaatagtactctccttt
tccttccctgctcccgactctccatttatatagacctgttactataagttattctgtaat
ggaaactgacaaggagactgggctcatcagctccaaatgtaaggtcatcactcagcttta
tgtttaacagttctttatgttaacctctgtagtgagaattttggtttattaaaaaaaaat
tcaattttgttgtgtgactatgttttttgttttaatcccaggtgtgggatcagaggctgc
ttcacagcaggtgattatgatttgccttgtgcactaccaggggcgtgttttttttttttt
tttttttaccagctacagatagtttaattctggggactctagagaaggtataaatgccag
atccccaagaaggctgctgctccttctcctgctcctggttgatattgttgtagtttgaag
agaagaagttggagaaatcttgctgatgaaaattggacttgccccccaaatccagcatcc
ctagtcaccaggaagtagtctattttgttggctgaaagaggtcttcacccctctcccctc
taaccttttctcctatatagtgttcagggttggaaggggttagggtggaataagggttgg
aagagaggtagagataaaagaacccaataaaatagtaaaaaacaaaatagcaccaactga
cctctttctggtcattgtgtttccatgtagagtactgacatctctgtagtcttcagtaga
agactgctgttataaggaatgtatttggtgtattctctgaatcaacgtcctgtttttggt
ttgtttgttttcgttgttgttttccttgtttttttttttttttttcttctggataagttg
ctcttgaaatggcatgcccttttttcagcatgctaatgctgaggggcagttgaccacacc
atcttttgtccttattctggtcctcagcagtgcagtggaagatggactacgtaatggtga
tagccatagtaatgattgatatgtagccatgtgtgtggcacgttgcttttgtcccagcta
ctttgtattttcttcccaaggatcatctgagctcctaagttcaataccaggctggggaac
ataggaaaaaccctgtctcagttaaacaagcaaataaggataagtcatttattgaacaca
tttctggggtaccaatagattccttatgtggatgtccttgctgagtctcgtcgttgccag
cctcgtgtctgagatgacttatgagcaggaattcagttgtcctcaggttggtagcagaga
tggtattgagctgagatttcacttcagcccaggttgttctccctgaaatacccagtgaag
cagtatttccaactttgggatctgcagactatggaatgaccataaacctaacatctgctt
tcaggtcctcagtgtcaccccactgtggcaagtttaaatacacagtttcatagcgcaagc
ctgaggatcccctgggtccctgaattactgtgttggtgacatagttaatttgatagcctg
tctaaacaattgattatttaaacaattggcctcttccttacccaactgaaattatgctca
ttagcataaattcttcctacttactttaatctcatgtttcatcggtgcgctcaccctggc
ccttcagtcctctaaccgtagaggaagaaatggcgtacactcaaaaggaagtctgtcctt
gagttaacaactgattctccagacttgtgaaatggcttttttgggacagtggatggggtg
gggctttcctacattagccattctttgtaacttttaaaaattaacaacctttttattttc
aaggtaatagcaaaattgagtggtaacacagaggctcctgtatgcttcctgtctccatcc
acacatatcataccccacaataagcccctcaccagaatggtagatgctttacaaatgata
aggcagaggccacacttgcatcgggtttcactcctggtgttatgtattctgtgggtttga
acaaatgtgtaatgatgtatttaccagtgtggtattatataaaatagtttccattgtcca
ttgtcctagaaatcctcaagatcagcctattcagtctccatccacccaaccccaggctca
ccagtctcttcactatttccacaacattcctatttctcaagtgccatataattggaatca
tatagtttgtagtcctcttagcttgcctttttagtaccattaagtttctttagtgtattt
tcatggcctgaaagttcactttttactgttgaataatattttgttgtctgtttatctatc
catccacctaatggggaatattttggtttcctagacttcggcaaccgtgagctgcagaca
aggagtagtgtagttgtatatttcctttcgaaaaggaaacctatttgccagggtgtcttc
tcttgcctgtgctgtttttgcgttcttctcactgtcagcgactgctctgttcttcctggg
gcttggcatcctcagggctattggcttgggctgcttgggctgtgttgctgtccactgtgg
gcatgtgcacgtctccagtcgggtacatgcatatgtgcatcttcagactgttctcttttc
ttttcttttcttttctttttccttttccttttccttttccttttctttttccttttcctt
ttccttttccctttccttttccttttcctttttcctttttcctttccttttttctttcct
ttttcctttcctttttcctttcctttttcctttcccttttcctttcccttttcctttccc
ttttccttttcctttcccttttccttttccttttcctttcctttttcctttcctttttcc
tttcctttttcctttcctttttcctttcctttttcctttcctttttcctttccttttttc
tggctcttttcagtttattgcatgaaggagttacactagtccaagttaaaagcagacccc
aaatgattacattatacaagctgtgaggtttttaaacttgtgacaagggacagaagggaa
attctactcattgcaaggaaatcctcgcttaagcttcagagagccacaagcacttaaaac
ccatgagccttcagctgatcgtccttagccagcccagtctctatcaggacctggcacatg
ttcttgcgctggtcaccctgtagctgaattacttctccatattctggatgctcaattaca
gtaccattgcaggcaaatttcttcttaaacaccttcactagtttctttttatcgtaatca
tcagcgatcccttggacagtggtaagggtcttcctgccctttctctgttgagttcttata
tggatataatcctcagagccagcaggatgcaggtcatcacccttacttacttgcatcagc
aaaggggtcgaaagagtggaggttctggatagcagacatacagtacgattccttttcctc
agtggaaatggcctgcgggagaaggcgggtggcggggcgggggggggggggggggcggag
cgtcgggaagcgagggggctcgagggggaggcagctgagtccttggcggcggctcagtga
ctggggctagactgttttcttagcaatgttgtttttcaagagttttatggattttttaaa
aataaaagttatttgttaatattttacaagtatttttggcttgtttgtggtgtgtcttct
tattctagtcatagtgtatttcagagaacctgcttaattttaatgatgtgcatcttctct
attacttcattcatagacaatgcctttcatgttgtaactacagtcaaggttatctgctta
ggtttttttcttgcgtgtttgcagtctgctttaagataatttttgaaaagagtgtaaggt
ctctgagtgatgatgttgatgctgatgtagaggttcaggtgccgtggtaccatctacagg
agactaacttttctctattatattttctttgtatctctgtcaaagatctgttgactgcat
ttatgttggtccatttctgggtcctcagtcctgttgcgctatctgttccccaattgctct
cttgatggctgtaactttgtgaaaagacttcaggataggttagtgtcaatctttccaggt
tgttcagtattatatgggccctttgaagtctctaccgtaacatttaaatgttggagcatt
tgttagtgtctgcagagtaactgctgggattttgtttggaacagtgttgagcatttttgc
ttctgctctgccttcccgtgtttagaactttagaattcctttatctgccttcccgtgttt
agaactttagaattcctttattatattttcagcatatggatagtaactcaaaagacagtg
caataatgaaaacctacaaaagagataagaatgttcagttttaaagacgtctacaattaa
tagatttagcagtgtccttagtaagttggagaaagtgatttttgcagtgaaacatgtaat
cattaaagtttcttcattgaacactagagggccatatagtaccttgttaatggtgctgtt
ttcatgtatttaaatatcttagtgtctgcctattatttcttgctccaatttggaataagt
attaagggaagttgtttatattgacatataatatttttgtattttttaagtcaagaaact
agcatatttcttattttgacacatagtaacaaaaatattcagtgcatcttgtacatgtgt
ataaactcactctctcctcaaggatgagtacattttgaaaagtccttaagaattagcata
taatcttcagtcagcagacctcccatgaccagtgattgccctgggcttcctcagcatatg
tgcaaaaagagcaggtccctggcgttagcactacagtaggctgaggacgcctcgtcatct
tgaagtaaagaatgcttgaccagttaattttcttgacttttcttagtttggtagtggggt
tgaaccataatgtttaggccctaacaaagtagagtgactgtgtgaagaggaacaggaaca
gtttgtttagacttttggaatatggatgactacttgtgaaagttcagagtctgatcagaa
tcgtgctgaaattgtcaacttcttgcctctgtttatttgatagaatgcttctcattctta
atttttgtattctgggggacgtaggacattctcactagtcagtgttcatgctgtttttaa
gaactggtcaaggatggtgaacacagtgggcgtgccttagctacactgtgaacgtgggga
agggatgggttggactctgagactgccaagggtaaaccaccacatccctgagcctctgca
cctgagctgtcattattaagccacttttaggttgacaatttaaattttctgggcaggtta
gtccctgaccatacatggttgttagagtcgatatggataaacagtggtgtctcgtggtgt
ggttacggctggtgtgctctcaccacacactcactgctctgtaccgctgctgttcttctg
cagaatacacacatgcttagttctggcagagtcatgtgagcagagcttaagtgactaaac
accttttgctccaggaatgaaattagttacctcatctattatgttggtgaaagttaaatt
cttaaatggctaataacgcaatccaaacgagaagagggatcggtatcccgtaaagccttt
ctatggctctgttttccagagcgccaccttcatttacatcatacacagagaaaagtagct
gttctcactcattgcaaaatcaaagcagttttcatccaacaaataggattaatagcagtt
ggatagaaacatagcattgagtgccacaactgtattatatggtctgttttcattttcaat
tagattattttcagatgttaaactgccaggtgatctaatttctttatcgacatgattatc
cttgtttttattcttttgtgtgtgagtgttttgcctatggaccacatgtgtgcctagtgc
ctgaagaggccagaagtcagtgtcatatttcctgaaactggaattgttgcttatcatgag
gcatcatgtgtgtgttgggaattgagcccaggtcctctagaaggacaatcagtgctctta
accactaagccatctctccagcccgcatgggatcccatttaaaggatgtatcccagcttg
cacaaggaatctgaggtggtctgtatttcggagtagcatggtaaaatggagtatatgtga
gaaccagcgcaatgggaataatgcagtagaagtgaggctgaggccagagaggacgaaagg
aacgatctacagtgagttggggcaagctggtaatagcacgtaggccgggcatttctgtct
tagggttttttactgctgtgaccagacaccatgaccaaggcaattcttttattattatta
ttattattattattattattattattattattattttaaagatttatttattattatatg
taagtacaccgtagcttacttcagacataccagaagagggcatcagacctcattactggt
ggttgtgattcaccatgtggttgctgggatttgaactcaggacctctggaagaacagtca
gtgagtgctcttacccactgagccatctcaccatccccaggctgggcattcctgaagttg
aatcccagtattcttcagagtgtgaccctacaccacctcaaacatctggagtgctaatta
ggaatgtaggtcggccaagtctacctcagaactacttagccaaactctagagctggagta
tgcatttaagggagctgtcctaacattcgtagcacactggttgtccatagagtggtgcca
taaaactgaggacagtgtacacagccctctcaatgaagaccaaatattctgtgtctgttc
tctctgcatgtctttctctcttactgggtttcatgagtaccttagcagattgtacccttg
gatgtataataagtgcattaaggaaacactgaactttacagttagatagctagtcaagac
caaactgttaactctttgatgatattaaggatgctggtagtacctgtcacatagtatcac
tgagagtgtttattgaagcctgaaaggtagccaggacgagtcctgagagtcttcagcagc
tattagctgattgtatttcataatgtcaattatttcaaagttacaagtttgtgctaaagc
tagcttgctgggatgtcatgcaaagcattgttggtttctaagaggtcatctggtgcattg
aaactgttctgacttctacctaaatgattatggcatttaacttggagttgaactgtaggt
tatttaagtttagttagtactaaccatgaagtactcattttgtaattaactgcttaatgc
tgaaactagaaagagttggaacttaaaatattaataaactgggggccacttgggaatgct
gagaagagatgaaaacctcggttgtcttcttgctatgctatggtgctgtggactcagtga
ctgcagtaactggaaggaggatccattcgatactcctgcaggttctgtccctgctgcgtg
cgtgctgcgtgcgtgctgcgtgcgtgctggagagaatgcccacatgttcacactcacttt
ctttctttttacctgtctactactgtgtggtcagtggagtattcacgaagcaaagttgaa
agtatgatgacagaaaatttaaaagtgagctaattttttaaatcttacattaaagtaact
caggtgaaatccaagccatacattttctatagttttatattttattttgaaaatagccaa
agtcttgctaaattggttcatttttagtagttatcaaagtgggaaagtgttaccaaataa
tctccaccctcagaatagcaactaatctttccatcttggatcaccttttgcccttttcac
cgtggttaaaaattgaaattaacactctttggaaagactacatagatgccatatggtagt
gaggactgtgtaggtgccatttcacagtgaaggctttgtaggcactgtatcacagggaag
ctgtatactcggagccctttatttgtatgtatactttgtcatcccccggcacattgaaca
ctcagcctgtacaagtcagagtgcagcctgaggtgaagccgtgggtctgagtcagcggag
gctggttagtcgccttcccatcatgcagtcttaggctttacccagattgctcttctacct
gagctgccctgagctttattctatgactgcagatcacttggaaacagcacagcagtgtga
aaagcaagctccacttttttttttttttttttaatgacagcctaaatggatgttccttta
aacagaaatgtctgcccattttttttaagtgaagagaatgtagcgtacacccttcagagt
aagcaatattagcgtcctactgcaaaaaggaatcttttcttaaagacagggatactatag
tgatcatctttgaaaatgtatatgctgtaggatgttgtaatatctatggtctccttcctg
cttcctttttcttacgtagaggctagagataaatagctggtgtcttaattactctccatc
tcaggcctctcactgtacccgaggcttgctagtctggctagccagcaagctttggggacc
ctttcttctccatttgggcactagcattgtagatgcttgagcccactgagcatgcaagtg
taagcatgggagttgggtgttgaactcaaggtccccatgtttacatagcaagcatttacc
cttatgtctctatctccacagcctctcactcacttcctcttaactctcaagatcttccta
cacaagcacatttgggattaaagaagttgagttaatttatcttttttgatagctaattag
tgttttatttattgaagaaatgaaaactcagacatctgctataaaattaatgttgaaatt
tatgagactgtcagtttatctaggaatgaaagtctaaattattttctatagtttgattgt
atgtttaaagtattttatttagagtaaatattttttgtacttaatttttttttattttta
ttttaaaggtgtgtacgcacatatccttccctgcagggtccccagaagcacagagtagtg
tgatttcctggcctgcaaacgatttgtaagacttttgcaggagaaatagctgaaaggttg
atgagtgagttgtgctaaaacttgcttataaaagtaaagggcagactttaattgtttatt
ttgaatactgtggaagatgaagttgttggaggctctttgtcgtcacatggggatggaatt
cttgcatgattgaaatacctgtaattacaatgacatttttgccatagttgacttaaaggt
atagctatattaatcaccagctgaattcttggtgagcagctgtgtaaggccagtttaaca
gctggggtatcccggcataagcatgagagttaaacttagtcacgaagtaaaatttgttac
agacacagaggactctatcctgggcactgctgagacacttagaaaacgtaatctgaaagt
tggactttagtttataatcgcaaatctttctttaaatgtctgataaaagaatgagaagtt
tgttttgaagtaaatagttcatagacacaaatattggattaaaattcaaagcttacatta
gaaaaattaaatctatcatctatccactttgtatgttattgcctaggtcattatggtgga
taggaaaagaaaataaaaatatatgattttttgtagtgttaattttttgaatttcatctt
ttggtcaatttttaaaacttttcattacatctataccttctttccagttaaaatattttc
attgtatacaactcaactacactacctaacacattgccacgattgcctgttttgaaagag
tatccatgctgggcagtggtggcgcacccctttaatcccagcacttgggaggcagaggca
ggcagatttctgagttggagcccagcctggtctacaaagtgagttccaggacagccaggg
ctacacagagaaaccctgtctcgaaacaaaaaagaaaaaaaaatgaaaaccaaacaaaaa
gaaacgtgtagaatatacactactggattgaaacagtgagttttgtttagatgtattcta
ttttaaagtttctataaatcacatttatttacttatttatctatttatttatgaggggga
gggacatatacatgaaggtcataggacaacctttaagagttggttctttgctaccatctg
agtcttagggatcaaacccaggtcgtcaggcttggcaacaagcacctttttttttttttt
tttttcattaaaccatgttgctggcccttggatatgttttgtaaagctaactttactaac
tcgtgataaagaaggctcacaaatcgatagtccagacacgcacatgagactaagaaagct
acagtatacactccagactgtaaagttttcaccgtcagtcttctgtcccatccttctctt
tagggaatctgaagtgtgggataagcagccagcctaaagttccttgtttcatggtggcac
tgaccctagaatttaaaattcacctgataatctatgtgctgtgcagttgcctgtcagctt
tggataatttagcctagagcggtggttcccaactttactaatgctgggaccctttaaaat
agttcctttaactatagtttaatatagttgtactgtggtaatcataaaattattcctgtt
gctacttcacaactatcattttactactgttacagaacataatgtaaatttataatggtc
ttaggcaacccctgtgggtttgtgaatttgtagctgctaggtctatttgtagaatgcccg
tatacattcggtactgtttcagaatccaagctactgtctgcttatgtgcttagtgctttt
gggttgttctttgtgcttagatatcgaaacttaacctaggaagttcaagaatacattgta
gtattgctagtacactagtaatggtgggatgccctagaaactgtaacttgaaattgtaaa
cttcctgacaagtactctgagcatttaaatgtgtcgaacttctctggaaatcattttgga
ctggaccttagctcaccctccctcatttttaagacagggtctcactgtgtagttctggct
atcctggaatacagtgtgtagaccagactggcctccaactcaaacagtccctcctgcttc
tgtcttctatgtgccaccactgcttagcctggactttaaagtttataaacacagttcttc
catttgatttcgttttatgaatactaaaggtataggggccacactgtatagttaaaattt
aattttatgctgaattgttttttctatagttgaggtattattaatacaagaagacagttt
attggtttgtgactgttccctgtttgtttcagtttctccatttttatgcataaatttctt
aacgggcaatagtgtaagagtcctttctctttggcactaggctatgaaagaccgaaaatt
gatttagcacaggttgacaatattctatatataatgttctatattaaaagaatagttatt
ctgacttatttaactttcagtagtgctattaaaagtatctagaattttctgtttcaccaa
gaacacagtggtgttaacaaaagtggctttaagttcacagaagcactgaataatactgag
atccaatatttttcccattggaaaaaatgtgaaaacttatacaaaatcagtttcttaagg
ctggtgcagtgtcactttgcatgtagaagctgaggcaagaggattcttggagggaagact
gcttgaggcctcgaatttgaggctagtctggacactgtaaggaaactagcttactgtgct
aatagttgaggagccatgcatactgtacacaatgcccttcatggagtgtggttttcttga
gtagctaatcacgtgaaccaggctcttgcttaatgtccgtggttgtgaggctctacaccc
actgtccagctcgtgcttcgttcatcagggctttcttggatatttgaagggtttgaaatt
tttcacatttccattctagattgacaacatggctttagaataagagttccacaaagtaca
gacacttggaagttagtagcttgcactgatcatcactgaagagctactttatatctttat
ccttataaaggcttggctggacacttcatttagtgttaggtttgctggagttgcagtgct
catattatccagttgagggcagctacttaccaatttaaaagagtttagaatgctaccact
tggtgcttctgaaaatgcagtttctgatatacaacagtgctcactttctctcctgtgttt
tttattgtcacaaataatcatgggaacatggaaagctcctttttccttcgtaataattgt
ttagctctttaagaactgaaggagaaagccttatgttcacttaaagttcttcctgtccca
gctcctttagatcccgtctttcttccccgcttacatcagacttcaaaagtcgcatagatt
ggcttatctgggaacctggatcttaacagaacatacacataaacaaggcggctttacagg
tctctgagacttttatgtgtaaatgtgagaacagacagtgtttattgtgctgtttaatta
atcagtgggctgagtaaaaattaaattgcaactggcctgtttggtatgttttttatgcaa
aataaattatattctcacagaatatacggctgtctttcatttttgtttttgtttaagcac
agagatatcatttagtttaaatgatatcttccctatagttctgaaaggaattagaagtgc
taattgttttcagagattatcactgaaataaattattttaagaataaagatctcgttctg
tataaaattatattataatattattagcaaaatagatagtatttatatctggggatagat
atattagcaaaaaggttattctaagaagacgggcagaatatgagtgagggttataattat
gtgaacaaaatatcagtcttcctctagagagtaaagtgagtgtcaaaggacacacacaga
ctgggaggtggtgacatttcttctggagaggctggtgggaccatagacatgtttctctta
actatcagttattaccttcccccacccccccgcccctggccctggccataataatttata
gatttccagtgtccttatgctgtcttttctctggcctacccctgggtctttgttgggaat
gatgttgtgcatgcactgccatgcatctcttagagcatgtccttgtttagaagtgacatg
tgactgtgatcatggtccttcataaattattttataagaaaataagtgtatgtgtgtatc
tgaatgtgaatgtatgtggatgtctgtctgcatactcccatgcagagacaggaagacact
gatgacttgtgcaatcaccccttgccttatttccttgagatacaagacagtctcttggag
ctaagtagaggctaccaatccccagggattctcctgtctcagttccctgtacaacagtta
ctgactcacgggaccacacccagctttttttgtgggtgtgtgcaaagcttcatactcagg
tcctcaggtttgaacagcaagagccctgaagtccactgagccaagtctccagtcccagtg
ttaaggactgttgttatattctactatcatttactttttaaacttgttactgtggttgag
caggtataacataaaaagtaatgtttttccttgttcacgtatgttcagtggcattaagta
cactcagaatattgcataacaattaccaccttccctctccagaactctcccatttttttc
ccctgaaactctgtagcagttaaacatacttatctccacacccttgtgctttctgtctct
gaactgaactgtccccacacactgcttaagtgtttgattataagtggagtcatgccacac
ctgtctctttgtaatgcacttgttttacttagtatgatagcctcaactgtcaatgatgtg
tcaaggctggaattttcttcttgaaggcaaaacagtattcaactctgtgtgtgtagcact
tagcttacttagattatttccatgttgtgattgttgtgaataatgttgctacactcatgg
tacttctagtcaagttcctgcttttaaatcgtgtgtatgtccatgtccatgtccagaagt
ggaaatttggagtgaccccttgactgtgggcagtgtttggggagctgccataccacttcc
catcgcagttacagtgttttccatcatattccagttcccctacatactgccagcaagttt
gcactttaagctacatttcaaatttgatattagagagaaagacctggtttaagaacataa
catccggatcgggctaggaataattcttgtgtacaaactgtcttttctgctcattagtct
acttacgatagagtaacattgagctttccaaagtgggaggggctgggggaagaagccaga
caaggtggctcaccctgtaattctagcacttgggagttagaggcaggagaatcaccagtt
ccaggtcatttctggctgcaggatcgaggtcgacctgagtggttcgtttaaggaaatatt
taagattacactgcaactaatagtttgagaatactttcaggtagagtcgttgcctctcaa
gtttaccttgggcgttagggagcaatggaaagttctttaaacgtagatgttatagcccag
tcctttgcgttttaggaagggttgtctaattgatatgttggaggggttaagcacagagaa
tccccacctaccatccaacatttcagtcacctctgtgtcagagtactcggtgacagggac
acactgccatgggtttcttagagcatgtttctgctgagaagtgacatcgtactgtgatca
ctgcctatcatcgttcattttataaataataaacaatttatgtgccttcagtcagtcatg
tggctattataatggactcaggaactgtttcagaactacagaggaactgagattgataat
ttctagtaagataatttgaggttattaacagcgagagagcggtagggtatgtttgctgtc
ttggtgagcaggagactggtctgtaaggtcagtcacttgatgcagaggaggctgaacaag
caagctgttttcagcatgcatcaacactcctgaagtttgaatagactcaaagcattcttc
agacacttatggtgacgtttgagaaaggaagacttaagagaacatagagttgtgtgtgtg
ttgaattttgaaaaggaactcttactttccactttctagggagcaccttggttatttagt
tggaacgctcccaggaccccgcagcccagccctgctttgctcctgtctgcactgctcact
tgctgtgggagctcaccagctgtttgactggcaggagcaggagtgctcacttcaggagtt
tataattcctggctattctaggacagtttgcacttcaccaagcctcagacaggtcaggac
actcgcaaagagaaggttggaagacaaaaacggcaacaggccttggattctcagcctttt
aaaactactagtcattttttcagtttggttgtgagtgttttagtaatgtgcctgtagtac
atattgagagtgttcatcaaccaagaggagtttaaaatgtcaaaacctggaaaagctcct
gtgaaccatggcttggttcctgttgatctcaaaagtgcaaaagaccctctgccacatcag
actgtgatgaagatatttagcattagcattattgcccagggtctgcctttttgtcgaaga
cgggtaagtctgtaacatttaaagcacgactgtttattaagtgctgtttctcttcctgta
aagcactgcttccgatcgcctgtgtgttgtctgtgaggagagctaacgatgctttgtgtg
gctagggaaaacttacacagcaagagccgggcaggggcaccctctctgcctttcgtctcc
tctgtttttcatgagattagaatcctgtaaacttatttcaaactggtaaatgtgaaaacg
gtatgtcttgaaatatgctgataaatgttagaaagttgtgtagcttttattttgctggga
ggccaaactgctgttggtatgttccttacatgtgggaacgcagcatctagtgcaaagaac
actccaggcaggtctgtaggcttcttgggcccgtaaagctgggctatttttacaaactct
ttcatgaattaaaacttcatgggttagaagagatgtcagttgttttctgaaacagaagcg
gcacatggttgggtgatatcccgaatgtcttggactttgcagcactcgtgttactgtata
atcagcataaggtccaagggtgtttttcagtatgacgcactggggctgtagtattagcaa
tatgtgtttcttctgacctagctgctgttgccattttgaattatttactagagtcatgta
tttatacatatgaaatatgtttatggtcctgaacacagtaggcccaaacacataagtttg
tttgtgtgtctatgtctgtctgtctgtttctttggaagtaaatgagaacttgtttaatca
atgttttctgttcttacatctcttatagttactttaattttttaactgcaggggacaagg
agaataatccacatcactagaaaacacttttgtctgtatagtggcatttactgagatata
ggaaagctttgtaatttttttctaattagcttagttccttttgagtgatttcaacttcta
tcttaataagtggcagccatgttggaaaatgaagttaaaacaatgtcttctaaattttga
aacaagaacccaaagtgctctcttgtcctatttcttctcttcttccttttctatttgttc
tgtcttcatggtttccttgtctttcacctgtcagtttgaaagctgctgtggagtgaggtc
actgctcgcatttgagattttacaagaagcctctgacagctgtaatgtcattcgtaaaat
aagcgattagtttactccttctgtaaaagaaaataccaacacaaataatttaaaagacaa
aaattagcaaacataaaaacatattaaacaattgaagtattaatagcgaagttaataatg
ttctcttctcttgttcttattttgtaaagaacatacggagttttagcttgcaggagactt
tatgtcacagggcgtttgtttggaaactgcacagaagctgatcctgaaggacccattgcc
gaggaccttgagggcttcctgcaggttttctcatgtcactgctctgacattgcttaggtt
acggccgtgcagctgcgtgccaagataaaactatgtatgggaataaaagttgtactagaa
aattagaaataaagaacatggctaacgatttgaaacagtcttaatcagaatgtccaggct
ttgttttataaaaacagtgttgagtgtggcatgactcattgcctctgactcctggaaacc
tcagagttaggagaccttctctttccacatttggttactttaaatggattttcattatct
ttgttagtttagatattttaagtgtttatgagagaactcttctggagtaccatatagttt
gctcttattttatgccagatgtcttggagagcatcatatcccttccagaggtgctgactc
ggctgctatttgtggggagctattgttttgaacaaaatcgaaagcatgacctatgaaaat
tgtgggaaattctttaaaactataattatagctatattttttaactttcatctctctctc
tctttaaaagaaaatgtgtttatcttatcaaagtatcataactatgagggtagtctaatt
tatcagtttttctaggctctcacctaatgtgtgtgaagattaaagtttttcgtaacagta
aataacaaataataaactagctgctaatcagtcactattgagtactttagcattaaggaa
cccatcagctagaatatttggtaaaaatcagttccttagggggccggagccatagctcca
ttgttaatagtgcagactgctctcataatagtggccaaaagtttggtccccaggcgtcag
atggtgcacagtctacctgtagttcatggcccactggcaccctctactggacttcataag
aacccacattcacattcagagactccctgtacacacacacacacacacacacacacacac
acacacacacacacacctctacctgcaaaagtaatcttaaaaaataaactccataggtaa
ttcttaatataaagaagatttggggctttaacttataattttaagaagctctgattatcc
tttaggagaacagaaatgatgtacgagttgctttattttcccttgaaactggaatgcttt
taattatggtagtaccatcagtaacattctaaaattccagtttcattatttttctacatt
ttggtcacatctaaaataagaatgcagaattgatttttctttttagcaagagaaatattg
gtgaatcttattgttagtatattagaagaaaaccattcttacagctaatattagaatgta
taaatctcacttattcactcattccagctttggaacactcaagtatctagactgttcctg
tagaatttctcttatgtgttactcttttgataggcagttgttggtttagatcagattctt
aaacatctggtgtgctcattcagcgtggaagactaagtggggtagggcttgggcgcaggc
agtgagtgtgggaaaacactcacaggtccttctctgttgttcattgtgtttatgttttga
gacagtcttactggacagctcaggctggccttgaactagaaattcttctgctgtaccctt
tctagtagcaggctttggttgtgtgtatcaccatgtttgactttaaagagcccagtaagt
agcctaggaactgtgttactgggtgactactatcatgagccatttcggtccttattgact
taagtgtgagctgaatgctgtagaccagggactcaaacaggtgagtctcccagtttttgt
ggtgatcctgagcagtgatcttacctcctggcattccactatctctgtggtctatctaca
tacaccttttcctaacatgcttggcacttttcagctgctgtgataattaggtttggaaga
taagtctgggagcatattacagtccatttgtaatatgcttaaaatgtcttgtataatcac
cttcaagatttttaacttaagctacacattgagacagttttactcattgaaattttgaaa
tgtttatgtcagtggttggattgttctctgttgccttgttagccagggatcacacctagt
cagctgagttcacctggcagccattgagacaggcagggctccctcacgttggcgcactgt
ccttcctgtccacggcctcagtcagtgctttggacaaggacaaagtgacacagcacctct
tcagcaacattgctttggtttaggatttgttcttaatcactgtgccattaatagagacaa
agctgtattaaaatttttagtgttttttcaaatataacctaatgggctgttggttttgta
tgtatagacttcagttagtttaggttgtatataattttaatcttgttcttttagtggact
ttagaccagccagttaatttttcagaaaaggcctgccataattattaagaaaatgtattg
ggtttttgtttgtttatttttttgttttgttttgtttttaggaagaatgtctaatgatcc
cgtttatagtaaaattttcatatgtatagagattttttttgtgtgtgttactaaaaactg
taccacaatcgtgaaggcctttcttggtaaagcaaaagtatgaattctgaatttagaaat
gtgatatctccccaaattatctggattgagtagcttatgtagtaaattttattttcattg
aacaacttacatctcaaaaataggacatgcttattgtatgcaaaataaaatactttcttt
ataacttcctaaattattagtaattttactgctcagaaacaaccaaaacaataattttaa
atatttaattatttttacgcaaaattggcagttagacccattcttactgtattgcttgtg
tttctccaagactgtgagtactggtaagtgtctgatctgccacgtgaatgtggcagtatt
cttcttctttgctaatttgaatagtgacatgctttggccatgtgtatacaaaaaattttt
agtcatattattatgcacagatttgtagatgtaggattacttggtaaagaattaacttta
ttctgttcttggtaggtagtaatgaattgttttaccgaaaattgtaccaattttaaaata
ccacagggcccatctaaccttactgtaaaacatatatgtctgtttattcctatgtaaaca
ttttagggacattacacttaggtgatgtcagttccaatatttccattaagtacatgtcat
catactccatgaagaaagcataagaagaaatctaaaattatttgttcatagtgatgatct
ttgttgtggaaaaggacctttgccagagacttgtcacctgtattatttccaactgtaagg
attttctactcttttcatagctatgtttctgtgtctttgtccaaatagattgtccacatg
tgtgattttaaaaatgtatttctgattcaacattgtctgtttattgctggtcttctgata
gatgtcttggtttttatgtcatattatactcttacactcctggattttagtgagatattt
ttatagtattaatggtggcctctcccaaagtgtactgctattaataatctccactacgag
agtccttctcctgcgagctgcagcagctgccggagacacttgcctgcttcttacccagtg
ttacttaggtcacatctgccatagctttgcccattagccactttctaggcttacccttcc
tccctctctctgctgagttccatttccaccatgattaacttgactatcttagtttcagca
gttttcttgtttaaggtggccaactgcaattcaacttggtttctacacagttcagtgtct
gtttccagaatgttcagctgaatatataaacacacacgagtatctcttcagcagagataa
atataaaacattttaacacacatcatcccccctgtgttacgcatggatttttgtccccag
gttctttcaagacctcacggtggagcctggctggctggctgcaccttcttccaggcgctg
tgcttctttgtgccctagtggtcctcgtccattatccttaactccagtgcactcgctgtt
ctaccattgcccgtggcctggtttgggctgctcagtggtgctgtgtgacactttgtagaa
acacttggtggcttcagttgggctatccactcagaccacaggggctgactccaatacttt
ttatgctgcaggggatacgcaggagaaaggtagtgtacagatttgtaggactggttttgt
tgaaaaagctgttaaagttggagtctcttctaggagttttcgtactcttaggagcatggc
atgtttttgtttactgtgttttcaaatgactatgcagctgtgattttgaatatttcattt
tctaaatttaaccaaactcagatatagtttgacacttaattttaaaagtaattactactt
accaattttgaaagtaaaggctaaaacaaaaactcagtgtagtcctttaggaggtacttt
taagagtcatttaggaggcatgagggcttgaaaatgccagaaaaggctaaaagagacaaa
gacctaatactcggaagacgaaggtcagtattgtggtaggagctgatctagaaacagcat
tgagtgaggatatagaaacattgtcttttgcaagttgcttaccacgtaatatgcaattta
aagtcctaccagtctttttcttcctgagtactttgtacagacatgagtgaagagagtaga
aatcaggagtttcctggggaaactacagtctctaacatgcaaggcactggctaaaaatag
aagctcctatacaggtctatacaaaaatttaaagcaaacaagcaatcaaacaaaacccta
caaccttttaagcatcttccgtctgtggtaaaacttaaactcattgtaatacaaaagggg
aaatgcctaagaggcccactgtacaaagaggccaacagttaagaaagggcttcaggacga
agacatcccgagttttgcaggcaaatagatggaactaggaaatatcatcctgagtgaagt
aactcagacccaaaaaggcatgcatggtatgtactcatcagtgatattagccaaaaaaag
tgcagaatatccaggatacagtccacagaacacaaaaaggttaacaagccaaagggccca
agtgaggacgcctcaatcccacttgagagagagaagaaagcaatcatgggggtggggggg
aaagggagggacatgggtgaaaatggggacagggagaggaagaagggaacatgaccaagt
attgggtgggggaaaaggattgaagccctgagaaccaacagaaagaatggaaacaggcaa
ccttgggtggtaggagatgggggtgggggtgggggactctccagaatataccagggacct
gggaggtgaagagactctcaggactcaaaggggagggaccttagatgaaataccctagag
tggggagagggaacttatagagcccacctccagcagaaagatagacatcaagtgagggat
ggagttaccatccaactgtttaaaactctgacccataattgttcctgtctgaaagaactg
cagggacaaaaatggagaagaccctgaggaaaaggtccagtggcaggcccaaagtggggt
ccagctcaaggggaggtcccaaggcctgatactattactgaggctatggagtgctcacaa
aaaggggcctatcatgaccacattctaaaagactcaacaagtagctgaaagagtgagatg
tagatatttacacccaaccaatggacagaagctgctggcccccgtggttgaattagggaa
aaggtggaagaagttgaggaggagggtgaccttataggaagaccagcagtctcaattaac
ctggacccctgagatctctcagacactggaccaccaaccaggcagcatacaccagctgat
atgaggcccccaacacatatacagcagaggactgccgggtctggactcagtcagagaaga
tgcacctaacccttaagagactagaggccccagggagttggggaggcctggtggagtggt
ggtggttggggacattctgtggagatggggggaggaggtactggatgtggaacagtcgga
gggtggaccagaagaggggtgggagagggagagagagggagagggaggattaaataaaag
taaaaacaaaaagaacttgagatatctttgtttttgaccagctgatctgagaatggcaag
gcatgtggactgttgcttgtgtttacatcttgttactttctgttagttgtcctgctgccg
atatccacctacattgtgccaataaacacaatacccaaaaccaaactgagagtttttctc
taacctttggattaaacaggactagagagaattctgtgttcttctggagcccacagatta
aatactcctacagatgctcctgttttgatgacagtaaagtgaggcagggagaacatctaa
tttggtataaggataagcagatgggatactactttataagtatttctcaaaaataccctc
gtatagtacgtaagatgacctcatgaggtatactgagttatagtattcctactcacttgt
atgagagatctgaggcctaggagggtttaagaacttgtcttaaaactagaagataagtaa
ttacttttgaatgaggtcgtcaggccgtgggactagtatgttagtaacctaatcacagta
gagtggattttcattgcactgctgctagtatcactttgatcttgcctttggtgtgatgcc
actaatggccactcaaagaccctgagtccatggaacagagttgagagccttgaatagacc
tcttcataaaacctgtgtccttaacgttcttcacataggatgatctcggtatgtaattat
gacattgctttctggacctttttgtcatcagatcttagtagtattgggttttgcttcctt
tgttaatctagacataaagtaaattaaatgagcacgtgctatacaaatcacactgaggaa
acactgagtcatgtcttattacccacaataagctggacagatgataagtagccttaatac
caacaggcagggtactcacacctgcaacttgcattttaagaaatgagttgaatgtcaaga
ctggtagagtgaacaacaaataaaaaaatactctaaaaataaacttggaagtaagttcct
ggcagagtaaattgtttgaaataagtctgcacggccccttgcctgcacaccagaaatcta
acaagtcctagaaactaagacttaaaagacttaaagttgacgtatgatggcctcctttaa
tacttatctaagtgattattttgctgcagaaataatgtaatgtgctagaagtttttgtaa
tatgtagtttatgtgccgtattatctttctgaagtgcacatcaaaatgcacataagtgca
ttatctttcaaaatgcgcatgcatgaatgcagctgtcttcagagttagagaaccaagcct
gttgaactgtaacattcataactatcatttatatgtaaaacttgaagactcttggagatt
tcaggacggtttgaactctctcttaagtctctgtgtatgtatgaatttaaagcatacatt
gcatggataaacacctaaatctattacctagttacagataggtcaatgtagtataatgac
agtttgcagttaaaatattttcatcagtgattaaaatatggcagatatatacattaaatg
atgggaaggaaccccagccacttttgacggtctgcacattaaaataagagcaaagctaaa
ttaagtttaaccttccattacagattatgtctgttatctgcattttgctagtattgtaga
tggataagacagagtggcctcccataaggaactactgatggcctttctgtctcttgagag
atgtatctcctgttatcgttctatagtcatatgtcaaaaacaccagaaggacaagctgga
tactatgtactgatgcaacacttggaattaagattggaagatatatgatctcaactcaac
ttgggctgcctagtgggaccctgtctcaaaaacaaaaccataggtacatgatttcattca
ttcatcagataggtattaaataagcaagataaccattatagttacaggatgcatttgtat
ttgggttgtgccaaaatacaatgtgtcagcagacactttgtgtgtgtgtgtgtgtgtgtg
tgtgtgtgtgtgtgtgtgtgtgtgtgtgtacatgtatacagggctccacagaggccagag
atattggatcacctagaagtggagatccaagttgttgtaagccacctaatgagtgcttag
agccaaactcaggtcctccacaaacccagcacacactcttagctgcagagccatctccca
gccctcttttattatacacttgtattttatgttaaagtttcttcacaagtatatacacat
acatggacagacacactttgaattgttgtagcccttagtaaaatatataatctgttctca
gaaattctagtacagaaaatacagtttgaattctattttaaaaaaatatacatgattttc
ctctactcatgtttctacagcaagactttttttgttagagagtctttagtgtcctgtcag
tcacatcccagtggtttgcctctgagtcatctctgggtcagaatcaagtcctctgctctc
cctctccctgactgtgcgaaagttgcctaacttactgtccccagcacactggccagttaa
ataataatcaagtaatccaggagtgttgaaatgtaggatacaattttctactgagataat
agcaatcacctatctgagataaatggatataagccaaaagaaacccaagacagaaaaggt
taaatactgatcctaaaaggacatggacagttagttttgggttgtgatatctttatatga
tggtactcagttctgttttgagaagaaaaaaaatatagaaatgttgaataacactgttac
cttaaatttcttccttacaggtcttgagtgtggaaattgaaatttgttattcctgtgctt
atttacgtttttgtgtgtgggtggttcataataatagtttatttgtgagcaatttaagcc
caaacagcttttcaatcatccacagaaatctttaccagtttagatgtaaaatgtcattgt
tactactacagtgaacatataggggcagtattgcaccgtttttgtaaatcttgtctgaat
ttgaggatttttaaacagttgcagttgcagccttgttcttggacaaatgataacaatcta
tctgacttttaaatgcatggttgcacctgctttgtgaagtccaattcaggttcttatgtg
gattatgcaaaactcttgttttacagagtccttaatttgtatttgaagtcaaggaaatgt
taatttaaagggtaaaattcatctgtagttagacaacaaaatgtaactaattcatgaaaa
attgcaaactattaaaatcagtacagtttgtttttaactgctttataagaacttttcaag
aatgtgagaggtttttttgtcaacttgtcaagcaaacaaaacctagaataatatatagtc
ttactttaagattcttcaattgataaatactatttttcaatatttaagtagtgagattat
tacaaactttgttatagatttttgcaggttttttttttttctgacttcagtgagagagaa
ctgagcagttgtttcccatgtatttgttgttctcgatgctttcttcctgtgatctgagga
ctacttgacacactgagaatcaacagtgaacaaagcaaacccaggcttcactgttggctc
atgtcagaagagcagagagattaagtatgtggatggtggtgctgtccaaacctgggcgtg
agctcttactctttcctttactaagtgagccttagtaaatcagctgagtaaatggagaga
ttactactatctcatagggttgctgtggtggcaacgaaagagtccataggaagtgtgtgt
ggctctaacaagagctcacgtcctcaagcccatgtggctaaagagcgagatgagttctca
ataccagtatatcttacttttacttctgatatagtaataattcttactatttttagaatt
attgttttgaatattttttaaattgtgagagaaaatgcatagcatccattttgaatagtc
agttattttttagtttcctgaatgactgataatccctgcctatagaaagttaatgtggct
tggttcgactcacattatgtagattcattcagtgtgactaccctgtgtaaagattaaccc
cattatgtctatatcgatgagaaaatgataggtgacatctctgtattttgtttgcagatt
cccacatgtttagaataagtgtgttggcttagaatcccattaagtagaatttgtacaagt
actaaatgagaattgaaaagagtttattggttcatgtgttctgtcgtttgcttcactgtc
tctagtcaatcccttccatttatgattttaaattctgaaaccctttctcttttttcagcc
ccttaactttggtaattaagaagtggaacacagttgatgacatctttgaaaatgttctga
tcaaagcggtcctcattactgtgtagagagagacactaaagcatttgaagaactcagctt
tctcatctgttagcaaatatacttaaacatttaggccgttctttgctataccttgctttc
ttcattcttttacattttagacgtataggaaccatgctttaacatgcttataggtatgaa
gccctgcacacacagggcgaagcattgttcaggtctgcaccctgtttaatagcttatcag
ctatgttcctgctgtgcctgctccccggaggggaagggttgttctgcaagctttcatatt
agctttgttaccatgcagtatttctcagctgctcacgctcattaccatacagctggtgtg
gagacgaggaggaagcaggcagaagttgtactggctacaagaataagaagctcttttgca
gatggggtgctggcagatggggcatgtgtttgagcaggttttactaatgacagtgcgcgt
taaatatcattctgaggaagtccaaggtcattgtcatgaatttagagctttcgaggttgt
gcagctttgtgcagaacagaccttttcagagccctaattaataaaaatgtaaccagtggc
aagcaacaactagaaacatctaatgagtttttctttattttagtatattttatctttctc
tgacatgatctaagaagcattaatcttatcaattagtctctcttattactttatgatggc
ataattttaaagttattaaaaatttcaagctatatacaaaagtatatgctttgaaaaaat
acagtagcactcagttttgctatataatgttaaactattagaagatcaaatggcagaaga
attggaaaactgtgtctggtgttgagcattctgaagattaagttgaagtgtgtgacacat
cattgtaaagttagacagaaatggtatggacaggggtgtgtcttttgttaaaatattttg
tgcttaattttttatacccataaaaacatatgaggttgagttgaaataactgccaacact
tgctttttaaaaagttttagccagatgagggagcacacaactgtagcatcaggacttggg
aggctgagacagtttgctactactctgtgctgcttggtgagtaccagcctagccaagact
gtgtaccacgactgaagaaaaataacaagaaaaaattcttcagtggcttctcactccttt
tgacccttttaattaactgtagttttttatatatcatattacaagatgattaaaaagaac
agctaaagcaaaatacagctgtgcagtcgtatgtaaaatgtacagtatcccctttaattt
taatgtaaatagaactcttttattcccccaccccccacccctcttgatccagcatccact
cgctctgggccctgcttgctccgctccggctcaatctggtccatgggttttgggtttttt
tttggttgattttttgttgttgttgttgttttttttttttttttttttttttctcattac
agatggctgtgagccaccatgtggatgctgggatttgaactcgtgatctccaggagagca
gtcagtgctcttaacctctgagccacctcaccagcccacaaatagaactcttattttgtt
aatggagattaaataatataaacacacattacatataaaatatacatagctaatttcagg
attcatggtcagagcatgtttgcttacaaactttaatgcttctattttgtgttttgtttt
tttttattttcttgattagtagaggcacaaaattctgttaatgcctgtaaataaacaaat
aaacaagcctgtatgagattttcaggaaaggttatactgttaaatactttaacagttata
ctttttttcctttaaacactgttagagtatgttctgtttattgtgaaggtttgttttcag
ttggctttttaaagttccagggagttttcagccattccattgcatctcatcacataccga
gatgtatttctgagacacataactattctctagtgagaaaatacataaaagataaaatta
taaataacatagaatacatataaatagccaacactttaaaattatttctgtaggtctgta
tttatttggtagagtgtatatatatatatatatatatatagagagagagagagagggaga
gagagagagagagagagagagagtatatatactctacaaaatatatataatttatatata
tatatatatatatataatatatatatatatttgtagagtatatcttacattgccaatagt
ctcctgtcaatattttttgttgacatgctatttgtacaacaagtttgggaagaactaaga
taacacaagcatcaactgtagtcactgctgcccatgtaacttaacattctcattgggagg
acagacaaacataatgtgttacagtgtacctaggttccagcttgactgagtgacagcttt
ggaatgggagacataaccagtatgcagatgacttaaaccatctcttcttcaacccagacc
ttctgagcttcagagccagttggtacatggtacatctgtctttggatatcttggaatcct
gtcatgttcctcctcctgtcttctagaaagcagaaaacaatcaaacagcagcaacaaaac
aatccttgttatgtcgtttatcttctgtctggtcaaacaaataagaaatttgatgccatc
tttgatactgccctctcctttgtttctatcatctgtcaccagtatgcccagtcagttttg
tgtccaaacacctcaattgttgatccctgtgtatctcttgctacccagctagtccaagct
gccattgcttcttgcttgtccagtgacaaatccttcttggagcctgcccatatcctcttc
cagtcactttacctgctttcaaccttgcatttaaaatcattttttaggaaagtttgtctg
attacatcttgctttacagaagtccaatattcattggattttatgttattaaacataact
tttcattgcttttatgttaaaataaaaattatttacaaagttttccataatctagtcctt
ccttatttttctacccttattctgtgccactagtcatgcaggctgttccctgagcagtct
ctgtacccccggactagatggaaccttaacatacacattcttcaaacactacagcctact
gcatacagcatccccctccctttctctatctcctctcctttctttttgctttgttgtctt
tctcaccctcctacagtataatctcaagccatgaaagccagtatttaattagctaataca
gagtaggtaataaaattaatgcatactgaatgaatgagaatgtttgttggaaattaaaat
agccatataagtctattggtaaaaattatacagcctagcaggatggaggtagagacagag
aggataatgaagtattggtttaacttacagaggcgcatatttactaacaatgatatttgc
tattattagaatgggacaaatgtgcctgggtcagattgacatctgttaaatgtggtttgg
ctcctagaaaaaggatttatgtgataaaatcaagaaacagggccagatccaggtgtgatg
gtgtctagtactggagagagagcagggcagaggtagaaggatcctgagttccaagccagc
ctggactgcatagcatggtccaggtcaacttgggctgcatagtgaagccttggaggtggg
tagttgcagaggagggaagcaaacacaaagaggtagccatattatatactgtgcttgtac
ttaaatgtcctatgtgagtccatagaggaaatcctgtgactctggatatgctacttatat
tgttagattgtatctgaaactgaaaatatttagtatcagatgaagctacaatctccaaga
gtatttggattggtggagtcaatgaatagcacttaaagctgttttctttggttcagtagt
agggtttgtcacttccatcagttgctcttgggatccagatccttgtagtaaggatgataa
aagccttgcagcttccttgaatgggtgtttcattagagatcttcagtgtgtcctggtccc
tactatccttcagtatttccatagaaaataaagtctaggaccatgaaggaatgagggtag
aacctaagttcttcaaagggctggagaaccagctcagcaggtaagagcacctgctgctct
tgcagagggcctgggttcagtgtttagcaacctcgtctggtggctcacaaccacctgtaa
ctgcagactaggggctttgacaccttcttgtggcatccacagatgcttgcgtactcatga
tgcacattggaaatgagcaggggcacacacacacacacacacacacacacacacacacac
acacggaaaatagataacattttgaaaaatttaaagttcttagagggattagaaaaagga
agcaaacttaatgtgttatgatcaattttatattttcttcctctgcccactagtaactgc
tgagtcttaatatatgtaataccaaggataaaggaagttgttcttagcaaggggaaatta
gcagttgtgtatcaactcttgcttcttttggtaaattcatacacgcagttcatcagtgaa
cacatctggctccctggtagctagtttgtcttttctctgtgtctgccttggctttggtgc
agattccatccacatatgatacctttgcatgctgacacagctcagaaaacatcctagaat
atccgaaataccaatatgtcttacctcaatttagagaactctattttattctataattgg
aggccaagatctggttctactaccatgtttttctggccctgggaaaattagtgaactctt
ggcctctttcctcatataaaatagaggttatcatcatagcactgtcttcataaggctgtt
gtacagttttcatgacccaatgaacaaaagcagctggaagaggcatgacttggtgagttc
tcttcccagtgtataccattttcctaaaagggcatctttgggtaaatgaattgcttattc
agactcaaaaagaataacatacatatatagcctctttcattgtggctactgaagggacac
tcaatcatattaaaatctagtgactattttccctttattgttttagagtaaaatattatc
acatgcctaactgcattatcatcatatgagctgaggaatattagttattaattatgccca
tttgtcttctctgattgaccagcctactcttctttgagaatgtgggaggtcttctaggat
agagacagctccatcttgatcagcttgcatcttgttttgcttaatgtctagatagctaga
attttgctgactttggctcaatactttctcaaatgtggtagatgtttaaaaaccaaaaaa
cccttaattcctaattaattttctaccttttccttcatcgtttagaacataagaacccat
tgtatgactgagaatgttaggctgaagttgatcctattgcagtagagtttacggatgaag
gatcactctgtaaatgattcctagttatcaccgtttcctgtgctacctgtgtgtaaacag
tttagtagatccataaaatacgagttttcaaatgagtagagtagtaaggtaaattataac
ttggcctggcaaaatctgtttgttgcaattgtttaataaaaactaggagagtgaaaatgt
tttagaaagaaccatttaatttcatctttgctgatggtgatcatgttcattatcaagtca
tcatctattttgaaatatctttaagattttttaatgtgctattttctctctataaaataa
tttttaaaggaaaggcttaccatgaagaagaaattatttatctgtgagtgtatgtcttta
tcttaagacctgacagatacctaaataaattattttaagattatattgaagttaattatc
tttttgatagttttaagtttgaattttagctacatctaatatgtgattattcttcttgta
aaattcacacgtagtgacttgcaaacttaaatttgtgaaaatttagaatttagttttaaa
atgttctagcgtccttcttgtccagatccagagcaggtctcagctcctccacagcatctg
cccctagtctgcccttccgttctacgccttcgtgtggaaaagtcaataccagtgcagaaa
aataatcctttgaagttaggtatctaaacaacatttaaagttaatttttagacatttggg
gtccccaaggtcttactgttagatatctttagagcatgtcttatttgttttaaagtgaat
aaacaaaaggactataacaaaagattatgtgtaactctactaatggcatgtctgtttcca
gcaatatgcttttgtattcaaattgaggagattctaaaatctcctgaataagcaggggat
cttgtaaacattatgctgctcataattcttgcaatttgttaaattatacacactcagtaa
ctgctttaaatgtaaagtggtgcttttgatatgactatttagcacctttctttttaaaca
taatggggtgatcactgggttcttccgtgctcacttggacattacattagatgcttgaat
acttaaaagagcaagtaagaactctgatgtacattcataaagagtgtggtgttttgttta
ttagcgtacaaccaatgcagagtaggtattcaagcgaagtaagtttatattcccagtcat
tataatgattagagtttcaaaaatatttttaacaggtcagttagggctgtgtagtgtgac
tctgtctcaaaaaaaaaaaaaggaaattgaataaaattatttgttattttaaattttata
taattactgaattggtgaaaaagaaatgccagtttctaaaagtttagtgtttgaatctga
ctttatgtatctaccatccaaatattgtcacctgaatacagttgtcaaagtcagttgagc
aacacatagcaagtcagagagttctagggctgatctgatgactccatgtcaaccaggcca
gagcaagtgcacttttgttgaaggtctcctttaacttggaggctctgcagttatgctctg
tgttcttttatggtcacagagaatagttcacgatgaaatgtcctttcagctccaataact
gaaactaagtgcatcttttattgtagtagacatttgctaaaacaaatactggcaatacca
taatagtcattggtcatctgagcccagtcgctcttttaaaaagtaaattatcaaatgcag
aagtcagatgagaatgtagaaaacagaagagcagtgagataagggctgtaatggtggacc
ggaagtgagttagcagaacctcaaatggtgctttacaagtggtgcacagcatttagggat
tgggggttacctgctcttcagttctcttcttggtccaagtttagtggtctttgaaatagg
gggttgattgagagctggtttcagggtgtggtaaatcatgacaggatgaaggggtaaata
atatatatgttgaaaacaaggcaaatactgtttatactttttaccatgtctcttcatgtg
ggtatgtgcacatgtgcctgcagatgaatagaggccagaagtcaacattgggcatagtgc
cttatagctttccatcttaaattttgagactcagtctctcacttagtctgatgctgattt
ggctaggcactctgggcagcaagccctggggtcttcctctcttcaccctcttcccccaac
actagagttacagataggcacagtctactttgcctgactcctatgtaggtgttggtgatc
caatctcaggccaatgcttgcatagcagagacctaattgtatctcttatttcaaaaagta
atgattgctttgtgaatgatcttcaactttacaatgctgagaatacacggaagaatatgg
gcagacagtatacagggaaattgtgttaatcattccattttagaagccttttaatgattc
acaactttcaaactcttgatagtggtctgcaagatccttagtgttctgatcttgaccttt
taccattgcttcagcctagacaaacttgtcctatccttgctgaacctgaacctgggctga
aaagttgtctcctaggtgctaccattccagattcctctatccttgtaactgctcacttgt
ttcatagcaccccgctctgtgtcttagcgctgataggagcttcctgtgtttgtttcctag
tacactgctcaggcatacacaaatgagtgctcaaaatgcatcaagcttgtttatttattg
acaggtgaggaaagaaggggaaagcttacagagcaatttagactgccaccattaacattt
ttatatttaggtttttcttctctcttcctaaaggggccattattgaacttcaaagtagtc
ctcctccagtttgatatgtaatgaacagtagtgtggtacgtgttttagaaagaaaggttt
ggaggtgtaaacgggagagaggcatggggttaagggctgatggtagaacaggatgaaaaa
attcaaaacaaactgagtttatgaatagtaagggtcctgttagtggaaatcaattagtca
tgttccccctctgcttccctgagatacattgaggagtataacattttaataatggttggg
aaatttctctggactgttcccccactactgtgttcaagagcacaacttgcttgtatgtat
cagttctggtatgagtgtatgttgtaacaggaagacctcttactagtcattactaaagtg
aaacaaacaaacaaaaaaaccaaagcagtcctcatgcctacttatacattcctgtaagtt
ctccttgtatagaaaggcttgtgggattctcccatgaattaagatagattggctaataca
ttaaagaacactgtaacttccacattaggtaattgagtcagtcgatggcagtacatggat
taccatgtaatgaaccctctaagagcagtgatgagaagtcttaccattattagttctgca
ggttagtaatggctgtaagtttacattttggttcacagaggggacctttttcctccccag
cataattttgtcttagttggtggggttctaacctagctttctctacccagtgtctgccat
cctttcctcattgccacttgcctcagacttcctacctgcagtagaggcatgcttgtaatg
accagggaaccagtcctctgaaagcagagcaccttttttttttttttaaagcaatgtctg
tcagcttgacccactaccttgtaatgggaactagaaagcgaagttgagctctggaaacag
agcccacatcctggcaggacacctgttcttactattgtcaccattaatggcagttaccat
ttgtagggtacttaatacctgttagacattcggagagtcctcacagtcccttagggtatg
taagtatctgaggagtactaccactcattccccacacaactcatgtgccaagtggccgaa
ggcacactgacgtttgagccaaagttactcctcagagtctgcgcctcaagttcatcacaa
agaaagattagattagatggctccaggtccactccatggtatatggatggcttcctggta
ctcaagtgttttgttggttcaataggtaaaacactccttttatttcttagtttttagctt
ttttagatgcctctttgtcatgactaatcactaatttaagaaattagttccaggatttta
aacatttgtaagtgaatacaagattggtattcacacacaagatttcatatacatgaaaac
ttttctcaagtagatttgaacttcatagccagctccaataaatatagacgtggatgaaga
aaatagtaaattgggctgtaagctttccactgaaggaaagtaaatttcatcttgactgtg
tattagttactattttgtttttcctaaagtcaaccttttatatttttatgacattttctt
tacagaaaatataagaataagtttacagtacagttttaagaggaaacaactaataacaca
tacatttaaggtagtggtattgtattttagatctaaagtgtgagatagagaaacgcaata
cttgttagtagattgagggtagcttgcatggattgttggcctagtgcacaggtgtgtact
atattgtgaggtatgctgacttcctaccaaatccctgacaattctacagaaatatatatc
ctagaagcaaataaaacttctagctttattttcttctgtgactgtgttaaatgaaactct
aaccttcattattgtgtgtaagttctgaatgtgtggacatttatatttttgtacattcac
atacatgtgcacatgtgcatattataatctttgcattgactttgtaattgattgtgccag
attaccagaggctgcatctctctctgtaaagaggcttttagatagtttgaacttacagaa
aaataaataaatactatttttacagttatatttagttaaataaaacttttaaagtctgag
ctttatgttcatttacagtttgatttactatggttacaatttttgtataaaatctttttg
tgtttttcatatttattttaaattatatattgtgtttaatgttgggtagctatgcattaa
tactatagtgttggtttagttggagggtattttggaaaacgagagttcgatatttttaaa
actaattttattatgggttctaaaagctccgcttccccaccctgtgtgtggggtgtgtgt
gtgtgtgtgtgtgtgtgtgtccatgcccttgtcctttcttattttactatacatttaata
gacgaagaaaaagtcttaaattatatactatcatgtgcttgacacattttaagagctggc
taatttcctaatgaatgtaggaaatgccctactagccagtgccttgcctactgcagtagt
gcatgcatgggtgctgagctgcacacctccttcccagctgatagcacctgcaagagaaca
ctcccactgactttccactttagtcaggagcctttccataggaaagttaatgacaacgag
ttagtaatgattcaaaaagtttgttacagcctcacatgcttggcattcttttctttgatg
tagaaaatttgagctgtctctaatctaattctgctcagggcaagacagctaactgtggta
ggtagtttcctgttcttttccagagtagaatgttgtcatgataataagatcactgacatc
aaaatacaaacacatcttcttactattataaggtagaaatgtatttttaactgttagcaa
ggtaactaagaaaataatattcatggtacaaccaatcctattacagtaaacttggaaata
ctaagtatagtagatattaagaaacatatactttatatattcatacatatgcctgaatta
ccaaattactcttatggttgtactgcttgcttgaaaggccaattgtttaaaactgttatt
tgttacataaatgtttttctggatgtcctacagataacttgagtgatgtatgaaccttcc
gggagtaattattagagagaaagttgtacagagataacattaggttgttgtggcagaaga
ctaccaagactagttagtaatgacttggaacagtaattgtgtgctgtagggaagccagga
gtctgaaggtcatgccaaggaggatagatgcaattcgctgtatgtgcatacttcacttgc
atcatccacagtagactgttttggtttgtttatgtatataaccaactaggtgtccagccg
tttgctcagagcgagtcatttgcttagactagcggtggttaaccattctatgccattatc
tcccagagcttgggctctggttcaaaacatctttgtgctgcttatttctggagccagcat
ccacttgtatacaaccaactctaaagcttgctaataggttttaatttaaataatgcttat
gcattgctagtacaaggaatatttcaaaagaatgggaaaattattgcctacctggaattg
catgaaaatttattttattataatgaaaacatatttactatgttgagatattctaaaatt
ttgtacaactgacattagcatttatttatctatagtagaaactcaccagctaatcttttt
gatgaacttctacttgatgttacagcataagacataaaatacaaagtaagcttagaacta
atagattataggctaaagtcccatgtttctgtccatccaggtgtaatatagatagaaaa
aagtatttaaaatatattactaaaacacctaaatatgatttatgcttgagaccatctgta
agctaagacagccagaaatcagacgatgtgaatgctttgctttgcctgggtcatatgtag
agtctggtagtaaggagatacgacgcatggttttcagtgatgctacttaaaaaaagagtg
ctggaaaaatctttatttttattttgttactttgaaatctgattaggaaattggttaact
agttttgcttggctagttctttacagttaaattagcattgtaaactaagtattgggagaa
ttgtttaaatatttggtaaatggaagtcatcattagtaattataaccatacatatttttc
acttaaagggctaagttaataggggccactcctaagtgatcaccttggcagatgaaggaa
gtgaaggaagtgggatggtatagactttccagtgtatccttttcatataactttatgagc
aagtttccttgtaacttcatgaccataatcagcagttatgtgaccatgagaactcctttg
aataaaggtatttctatctctggcaccccactaagcttacccaatacaaattctatagga
gagtccaactacttgttaatattcttcagtagcctggaacaagcaagaactccatatttg
ctgcccccaccccacaacccctgatcttgaagcaactagttaataagggtctgtatattt
tcccaaatattggacacaccctcgcccccaaagcaagaagtttcaatttaaaaagaactt
ccctttgcagagagtgaattgattgataggaaaaataacttttgttgagcatgtatgtac
atattgtgtctgatggggggtgacataagaattaggtaaatgtttgagaagataatagac
agtagaacataatttcttggagagaatctaagatgtgcatcttcataaaatttacatgtg
gaagtaaacagtattacattgaatgtcatgtttataatggtaaacattttaaaaattgat
atttcatgtaatattccattaccatgcagaactactaaaatatattgttcaaatagtagg
aatctattttctaaagaaactaagtgctctaagtaactatgggcacatagcttacttgta
ataaagaacattagtggggaggataggagttaattaactgggcactagataatagctttc
ttatccaagaagtcttatcaccacttccaagggcagttgtgtagcaagttgcaagatgaa
tacctaaatttgtacataatcattctatgagaaaggttgcctttaggcaaagttagtttc
tttattattacataaaattcttttttaatgctaagcaatgtgcattgccacaatcatttt
tgaggtaaaagtctaccacctcttgttggcccagggtgagtgaatgtcaggggattccca
agtgagcacggagcacatggagaaagtagccttgatacaaacatgcattaggtagaagtg
gttcatctttaagggtaacccttcttgttcccgagaatcactggcagttatcttgaaaac
tatattttaaaataatgtgtggtaaagcttaagaaataattttactaaatcataatataa
gagacaatcaggtgaaatttatcttttattacataatttctgtaaaagaaccttttgctc
caaactggtttactttgttgtcagttcccccaagaatacttaatttttccctttggaatt
gcccatatcacactgcccaagctacttgaagaatatagtcagcctctgccaagtctatcc
agtaggatgacagtaatgaaagtagcctgcttcgcctctgttgggggtgggggttaccca
aatacatttcatttccaaaatgtttctttctggaaggacttatttgtggtaatgctgatt
gctatttaaatctcgtcaaaaatgtgggttttacaaaacacaaaaataatctcctgaaag
tttggaaggggagaggggctgtggtgtgggggcggtctctgctgcccacgtccgccctct
gttcagtgcgttgtgtgcttacgggctccctggagcagatcaccatataattgatgtgca
gtctgcattgctgaaccctggactgcaccattctgtgttcaagggaagatgtaatctgat
ccctctgctgctgagggaggaatctgttcagtcaaggctttgacagggcatagtctcctc
caataatcttgtgggttctcgctcattattccccgagttctcctcagtggagctgcatgc
gtgtgtgcgtcccgagcagcgttctggttctgagctgcgtctgctggtgctggggagttt
tgctgccggttccgctgcctaatcttccttttctggcgtgcctgagcatgtccacgttag
aatctgtgacatacctacctgaaaaggggttatattgtcagagactgccaagcagccgga
cacacgggggcacagaatccctgaaggggaaaaatacagaaaatatgggtttctacggca
cattaaaaatgattttttacaaagtaagtaatctgttttcttcctgtgctgtgtggttgt
gtgtgtgtgtgtgtgtgtgtgtgtgtgtgtgtgtgtgtgtgcctgagaaggaaaatctgt
atcatcagagatggctgcagcatcctctaattttggtgggtggttggattctggtattct
gctttatttcatgactgttgtaagcaagtatggagcgatggtgtgagctgggggaagctt
taaaataaaatgtacatttccatctgagattttttcactgaaaacgtatctgagactcca
gaaatgtagctccgttaaatgctgacctggaaatactcagcctcacttggctttccgtga
acctttctgttcagttttcctgtaccatagtcatacgtatgtgttccttagttaactctt
ggagtgtagtcactgaaacgtgtagggtttatgtttgaagagattggactacttgtggta
tcttaaaaaaattacccaattaatttttggttataaagtcagtattttcagttaacatta
cagtatttaaaagatttccacagtcattcagaagatgctttgttttgtaaaataatttaa
acataaacccttctcagtgaattctgtgatatatgttttaaatattttattcattgaaag
attctagatggaaataaagtttgcttttagtggcaattagatgtcatttgtaccagcata
tttacaaggcgttctgggaagttctgagagccagcattactgacttaattcagatgagtg
tcagagactcccataatgatcaggaaaatcatctgcgaaagttaccaagctctgtactgg
ctgcccttcctgatggattcatttgtgtgtaggtacatgtctgagtatctgaattctaca
cgtggaagcacatggctccctgtgcattacagcttctatattttttgttttctgataaga
ttatttacaaagagaaaaaataatatatttggagattatcttttaaaaatgaagtgagta
tagaaacttccagagggaagaaagcaggccaattctgtttcccacagtaggtatttcact
aaagtataaattgtgcataagcttcttgaaccttcgcagccacagatgacccagtgttga
ctgggacactttcttcacgataacaagtccttgctatcccatcccagagtttgaatcttt
ttaatgccttttagtatttacagtgtatatactatgttccatgtgttcctgtgtgaagca
ataggtaggaaaatgagcctactgcaaaataggggttccagcttactataaaaatgaaga
tagctatctccccataacatgcagtaatacagtagctagtgagaccctgcaaatccaaga
gaagctgttgaaataggaaaagttgaaccttaacagattttattttgtatacatatactc
agatgacctacgttaattgatagtgaaatacaaaaataaatatttcaatattgacattga
caaatgcaattttgaatgtattgatagcacaacatggaatagtgagaaacctttaagatg
tacatgttagacatgtctcatatatcatatgtcttagtcataactcattcttttacaatc
atttcgttgtttcaagagtattttttcctgcgtattctgtatgtagcagttgatgatttt
tttccttcagctttactgtggcagatacagattgcctgctattagggatttactcttttt
aactgtctctgttatccttgctaaactcttctttattttaatcactattcaagattagct
tattttgctctctacctttgtataatgatatgaatgcttaaaattcagttattcatattt
ctggttgtgaaatttaaaatttttatcttactatttttaaagaaaaatgattagtaagga
agacatactaaatacagttattttctatgtgtgctaataaattttttcttgttattaaac
tgaaaaatgaaaagtatataacattctaagggcatggtaaatattagtccctcacctaac
actgctcttcccagtagaatactaagggaacattacctatccagtccataagagtaaagc
aaataggaaaaactgcgtaatatattttctgattaacctactaataatcatcattttaga
cacatgaaaatttaacagtttttaaatattagcatgcaaactcaaagtctgataatggac
caagtaattttttttattttatctgttttactttatagatgaaaagaaagttggaccatg
gttctgaagttcgttccttttctttgggaaagaaaccatgcaaagtctcagattatacca
ggtaacgttgaatagaggggtaggcgggcatgtcatctggaaacatttctttgaatttgt
tttcgaagaataatagtgctactgtattcagtgttgttggttgttgaaatgtgttttaac
attacagaaacggggaagggcatgaggctcaacccaagcagaagggctggcactgtccat
cttggactttctcactgatactctgatgggagacacatgctggagttctttaaacaagat
tgcaaagtcacaattttaaggacttctaaacagtaaaccgcgaggaaatgtctctggtta
ctatagcaactgctgttttagggtagctactttcatataccaaatcgaagataaatttat
tagcagtgaggtaaatctcccggttactaaaacttacttcatggaatgaatgtattagaa
atccccatttattggcatgatgggttttatgttttaacacatacatttctacacttgtag
aatagtttccataacaactaaatacaattgtagtcttgtttccttcttcactccatcatt
tttggattttgcgtcagtataaaactttgcccactctcacgctaattagactaacttaag
cacaatatctcatttcccagccaattaaacatggttttggatgtcacttgaaatgctgaa
ttttaacatttaaatgacaaaagtatctaagaactctgtggtcccctcccccacttttgt
ataactgtcacttttattgaataactggtcagagtgaacagtagaaattccaaggacatt
atggttaggatctttttttaaaaaaaaaaattttgatttagttatttttattctgtgaaa
cagaataaaattaacattgcctagatacaagattttctaaaacatcattacaaattgtca
cacaccccatacatacagtttgtttttattttgttcaattgtggaaatatatttaatgct
cctgggtaatcaaggaatcatactgcttaggataactttacatacagaaagctctggcaa
gatgatgctaaagagatgaagaaagcttcgttctctgtacagcccttctcccctcccctc
tgctgtctgacaatttcactgttgggcgcttgcttccaccagagggtgtggagggataga
gagacaccgcaaatattcacgtcattttggttttccttctaaccttgatgatatatttct
cagatgaggaagttgtttgatgttaaactaattgtaaatgtaatttttttaatgcacaga
tttcagtctgttcacattattttggtcttctattaaagacaataccaaaacaaaagtatt
aaatgatcagctgttttagaataaaacttttggtacagaacacattctcataaaattttt
gttgcctactttttatatcaagacagcaacagccttttaacaaggaatcctactaaagga
gaattagagtcaatacatatttatttccatgtctttgacacaattttgtcagttagtaac
atatatgtttgatattcagtaatgattctgacaggtttttttttgacaggtaataagata
aaactcattttgtattctcctgcctccttaggagactttaactattaacacagcctgttt
tattttggagaaaaagaaactgaatttgcttttggatttgaatgaaggaaagattgattt
gctagttgaatattagagattgttgtaaataaatattaaaaaattcaaagggtgcaggca
taaagatttttctataaaatagtgatttatgtaccgttttaaaaaattaagagtttcttc
attagcgatttgtcagaggttgaaaatcagttgttatggtacaggttacatgtgctcttt
gtgtttcttttttaattcttttctttttggttttgttttgttggggtttttttgttggtt
tttttttgtttgttttgtaaacaaattcactgtaacccagcctgacttagaattcactat
gtagcttgcctcaaacttaaaatggtctttctgcctcatcctcaagtgctgtgataacag
atgtgagctgtctgctctttggatttaagatactttattatcatagttcagtgagctgag
gattcttccacaggtctagttttgttgaacgttaataccacctgtaaggcagaaatgctc
acattacctggtttgaggtgcttgaaccagcttagaggagccaccagcgtgtctgtgtgt
gtgtgtgtctgtgtgtgtgtgtgtgtgtgtgtgtgtgtgtgtgtgtgtgtgtgtgtgtct
gtgtgtgtgtgtctgtgtgtgtctgtgtgtgtgtgtgtgtgtgtgtctgtgtgtgtgtgt
gtgtgtgtgtgtgtgtgtgtgtgtgtctgtgtgtgtgtctgctacagtctcagggctgga
gcagaagcactatactctgttctgagttcatttatttagtgtttttcattatctcagtta
aattattggataagaatgtttagttatctcttttaattttgtttgaaattctctatactt
tttttgaaaaactatgtctcatcgaggctgactactaccagatacctaggctcagcttaa
taaatgacaaattaaaattagcaatggataaaatatcataaataatttcaatgcatctgg
ggcaagcttaagaacttatgtataaccagtgaaatataaaatattttttttctgaaatat
tggtaacaatactttcgtacttatttttacaaagaaaatctaaacttaaactgagtgctt
gaagacgagtcatggaagtaggagctgtatgttctaaagccatgtatttttgcctgcata
cttagttatgtgctcacttacggttagggctggacctggcctgtgttctattactgattg
cacctgcagccctaagcaaatacactagcaacttagagctttgtcatttggttgccagta
gcattgctggatggtgataaggaagggactttagtgaggaagaggaagaggaaaaagagg
aggaagaggaggagaaggaagaagaggaagacaaggaggaagaggaggaggaggaggaag
agaaggaaggaggaggaagagggggacgaagaaggaggccaaatgtgatggcacatgcct
gtatttctaacactgaagagtttgaagcaggaagcttatgagtttaaagctagcctgggt
gacacaacaagaccatctgcagagcaagagaaaaagaagaatagcaaagcaggctttggg
tgtccccgccctgagagtagcaagcgaaagggcttgctctactgatgtttgaagaacacc
agatcattattctgagtaaattagagttcatagtttttaattaagacctttcttccactc
ttacacctaggctcagcttaataaataacaaattaaaattagcaatggataaaatattgt
aaataattttaaatacatctggggcaagcttaaggtcttacgtataagcaggaaatccaa
aatttttaactctgaaatattggtaacattaatactttcatactttttattttattttat
tttttagtaccactggccttgtaccatgttcagcaacaccaacaacttttggggacctga
gagcagccaatgggcaagggcagcagcggcggaggattacatctgtccaaccacccacag
gccttcaagagtggctgaaaatgtttcaggtgactacgtgaacacactgttgaaatgctt
cgctcgtttgcttggggtcagggtgggagtacagaagaatttggccattaacaaaaaaag
tagtgtgtctgatgctctaccatttatgagcattggaaacattggactaaatgaaagaag
ccagatgtaagcggaaattttattaataaaaagggacagaaagtaggttagtggttgcca
tgtaggaagcagagaggcctggggctacctgctaagagatgtagggtttcttttgggaag
ggtgacagaagtgttctagaactgggtagagtcgtggtcatccaaaaagacaggcatcct
gggacccccctaggtgtacacttaaatagcacgcctgttacacggcatgcatcttctaaa
accgaagcacagtgcttattttgtgtaagtgtatatttgcatggctttattggttttcta
aatagtacctttattttcttgtttgtgtttgggtttctatgttgtggaatgtattcttgt
ttttggagcacatttcctgtgtattaggaatgtgaggttagtttaggaactccatcagtg
agtcgctgggattggtaacttagcattcactgcttctcggtagaggtgactcagtgcttt
ctttcattgccttgcttgcagatgccttgagttcgctcaacttccaaccactgtctttta
gtaggatttaaattctttgaacatttactgggaagattgccttgaatactgtggattttt
ttaatttaagttagttagattgtcttatctcttcttattctgaatgatagactaaatcaa
gaacataccaacacagctatcatagatgaattacttaacaattctttccagacagtaatt
aataaaaatatgaacatgcctgatgctagctagccaccacaccaatataaaatgaacaca
tttcactacctcttgtctgcttgaattgttgggtacataatgcacacataagaaaattac
aaatttaaaagatacattggctttgtcatttcaaatatttaagaagtcccattgtataaa
tcctaatattttaaatacttaggtgctgtgtgtgtttgtttcaactatagctagaaaatt
ttcttgaagtgatgtctttttaggtgagttttaacactactttatgggatggggcagatc
tagctactgtaactgacaggtgtggagccctggttctcagtagggtttgttttttgcctc
gcaaataataaggaaggggttggagccttttgatgtttactgtgcaaaagcaatatgaag
gatagcctccaaaagcaaataagtatccaacacaaaatattatagtactaaggttaagag
tcttgggattaaacaaatggaagttacattttacttatgaaaatgaatactaccacaaaa
aacctgtttttaatgtttatagacatgtgttaattaaactcacctttcctgtaagggtta
ctacttccccactcttttacctcaaatttatgacattttccccctacctatgccctgcaa
attctgaactttttattctgtccagtcatttagaaataaataaggcttgaaataaaaatc
tagacatgtttatatactcttaaaggaatatttaaaatactaatatctgaccaagccagg
gaaacgggaggaatgacaatgagttctgcatgcacatgaacatgtgcacaagccagcacc
cagaacacagccctggggtcttacctgggtgctgaaaccacatggtgtctctagtgccca
tttccacagccctcaaaactgactggaaaagacactcattagcaggctattccaaataag
gttttttatatatggttaggcccatttttcctacagcattttaatgtaccaaaatatttt
ttgtaaaccatttatgatgttcattaagagaattctttctaatattgatctgcaatagtg
ggggtattaatgctttcttctcatgaggctgttttagagagccctttatacagtggttat
ttaataaaatttccaaatgttatacttgttacagctcagctttagaaatgttaactataa
aagtaaaatcagaacacccaactgtgagtctctaaacccatttggtcctagaaaagtatg
tcttttatatgagtagttgcagtgagagatcattcttttattttatttattcactttaca
tcctgattgctgcccccccccagtctctccccacctccctccccttttcttctgagaaaa
tggagtgccccctcccccccgaaaacccagcacatcatgtctctgcagggctaggtgctt
cttcccccactgaggccaggcaagacagctgagttggggacatattccacagacaagcaa
cagctttagggtgtagtaggattgaggaccctgaaggggataggaactccacagggagac
caacagagttaactaacctggactctcagagactgaaccattaactaaagagcatacatg
ggctggacctacaccccccaccacacacatgtagcagaagtcagcttgatcttcatgtga
gtcctgaataactggaaagagctcatttttagttcatagaaacaatgtagaattatctgt
gcaagtagaaaacgtaatcctcctgacccaagacattctgtagcaactaaacgtcaacct
gacaaggtagatccagccttgatcttctgagcttcaagtctgtctctgacataacccagg
actcgttgccatacactgcccttgtcttagctagagttttattgctgtgaagagacatcg
tgaccacgataaaggaaaacatttaattgggactggcttacagtttcagaggctcagtcc
attgtcatcatggcaggaagcatgacagcatgcaggcagacatggtgctggagaaggagc
tgagagttctacatcttgatctgcagtcagcagaaagaaactacatgccacactgggcat
agcttgaatagataaaacctcaaagcccaacccacagtgacacacttcctccaacaggcc
acacctcataatagtgccactccctatagccaagcatccaaacacctgagtctatcagag
ccatttcccacattgttctactggctaaccagttttgtttcaatttgacataagctagtg
tcatgggagaaaggagcctcagttgaggaaatgcttcaatgagatccagttttaaggcat
tttctctattagtgatcactggggaggacccaacctcttgtgggtggtgccatctctgag
ggtctgtaagaaagcaggctgagcaagccaggggaagcaagtcagtaagcagcattcctc
catggcctctgtcagctcccaccttcaggattctgctctgtttaggtttctgtcctgcct
tccttcaataatgaacagcaatatggaggtatatgctaaataaacccttccctccccaac
ttgctctttggtcttgctggtttgtcacagcaatagaaaccttagccaagacaaccctgg
ttttgttgttgttgttgttgtttttgtttcgttttgtttcgaggctggtgccaaactaac
tagaactgactcacttgcctggaggcttatgatgcgtcagcgccaatcttatagatagcc
ctagctcccgaacaaaggagggtggtatgagctgacctatgaggggtacactggcaatct
ttgtacaaatctgcgattgaggcatttgagggtgacttccctgttgtgtaacaagcaaaa
tgggagctcttgtcctcttcctgccctcctattgctcctaaggcagtgttacagcacttg
gtccagcctcctctgtcctagtaaagtactctcaactccatttgcaaattgagtaagaac
aaacgatacaccactgtagataacagcctgttaatgaaaaacagaaatgtcaactaaaaa
caatggactagaaaaaatacttctgttttgatatttataatgaataaactatatttaaag
atttaccatgttctatgctaagtcaaattacacatgttttctgacttgttttacttaatt
taagtcttgttagtaacagaagattaaaagctataagagaggtgacaaaacaagatcagt
atttttgtttctttgaaattttgttacagcatggttaaggaagcttattaaaccttgtgt
aacgaagttgaagaactttgtgtatttagtcagggaagactagaggaaagttctgtattt
aatatagtctttacatcaaaatagaaacagattgagccttgggttctttggaaaggaata
cctagatcacgttttctgagttggattatgaagctgtttgaactaagaagaattgcccac
aggagagggcaagaactgttcaaatagagatggggaaactgggtgtggtggcttaagcct
gtgatctccagtgcaagatggtgtctcaaaacagcagtcaggtagactgactgccctgct
taggttctaaagaagtgagttttctatcatgggagttcaggctcagtgctgtccaaggtc
ttaaaaccttgtgcctgattggaacagtaagcacactctattagtgttccctcgtgctaa
ttcatgaatcacctgcattgagagagagagagagagagagagagagaaatcagcagggca
tgttgatacatgtctttaatccctgcactctaggggttgaggcaggcagatctctatgag
ttggaggccagccttgtctacaaagtgagttcatgacagccagagaagcactttcttggg
ggttgggggtggggcatctccttcgagtaaactgtgggatcctgccagttttggggaaat
cactgtgccagaaaaagtctcttattatggaggcttaaggctgtaagtagaagagggact
ttgcctgggaaatagaataggagagtaaagataggaagcttgagagaccatttggagatc
tgaatatcataatctagaagaagtgctggtagcttagctggtacagtaatgactaagtct
tagatatgttttgaacttcttgaatttgtctctgagcctcctgcatctgcattctgcttt
caaatgcttaattaaaattgcagttttgttgagttagcatagttgttcatctcccaaaat
ggccttggctatggtattgtctacagatttcttataatgtaggataggtaaaataaccaa
catactatttttttaaaaaattcaaattctgtgagggacgtgaatcaataaattgtaatt
ttaagttatttatactttatggtcagtgatgaatcaaatttgaccatgttgtcagatggg
cagaatctgtggcaatgcctgaatgctctgggatcagacaaccaatccagtgctttggct
cctgggtggggcgttcttcgtgttgcatttcaaagctgtgttgtgtagagattgagcaca
gagccctgaggacaagtgaatgctttctacattgtatgtgcaagagaaaggagagcacat
gacttactggagcccccccccctccttacactggcaaagtagagtaggtcacggacatag
ttcagtgctaacccgagttccatagttgttcacatcactgcagagtctaagggaatacat
acctcttgaattactctgagtgccgcttcctgcaagcctttgtgtttcttacatcttatg
ggatccgagtgcaagttttagaggaagagccataccttgcctgttactctttacccatcc
atgtccaaaatggtgcattagcccctagaggtgtcttcatcagaggcctgtgttgatatg
ttttcacgtaacaattgtgaagttacttaaatgttttacatggttattataaaattccaa
catcttaaattattcatatggccttaaatttgttcctttaaacagccatggcttttgtga
gcagtcatgctcactgagggaaaatattaaaaacaaacaaatgggaagaattttttaaag
gaacccatacttttgaagtatatgtgttttcatttttcataagtacagcatttttattca
ttttgggctttgattatattcaaattttgcagtattaactcagctaatattttctattgc
ttgccattttttaaagtaaagcatttttaaagtgtgtagaagatacttgtcagatatgca
gaataaccaactattaatttttttatgttgcaaatctcttagctcttagatgaaaacctt
attcaagccataaaattttatggctgtccactaaagagaatatatagagaaaaatgacct
atcaaaattccctatttattcagtaacccacatggatgtttctaggacaagattttcgtg
tctcccaatgtcttggtgttagaagaatgcataactctcataattaaaaataaatgcaga
ctttggtttttttgtttttgtgttggtttgtttgtttgccagtcatgatttttaatgctt
tttgggaaagttactgagtaaacttgctgaaactaaaacctgcagtcgcttcagtggctt
ctgtccatggttccactgctcaaacatggccatgcaggggcttgcgggtcttttgtatga
atttgtaatgtgtccaattcctctttggtagagctggagcggaccagagaagttgctggc
tttagatgagctcattgacagctgtgaaccaacacaagtgaagcatatgatgcaagtgat
agagccccagttccagcgagacttcatctccttgcttcctaaagaggtaaggggctgtca
accgcatcccgctgggtgctgtccagtggttatgggtgcataaccacagcctgtaagcag
tgtcctgctcattatggttgacacttggacaaactgctatagccttttggtacagttgtc
acataaagtgcattcataccatggtactacttccttccacttggataaagtaaggctttg
tgtgagagtcactgagtttcttcaagaattttttccctaggttatctaaaggctggcttt
agttatttggttatttttttagttatttagttgtgtttattatttatagtggatgttttc
aaaattctgggttcattaagcagcaacattatttttgtttttattttgttaattatctaa
tgccccttcaaatcagaaattttataattgttgtatttctctggttattacattatacaa
tttttttaacatatagaattttacctatattcctttttggcatttctactaatcatcatc
catcagagagagaaagagaggagagagagatgacataaaaattggattcttacagctctt
aatcatatagccatgtgtggcttatagtttaaatataatgcacgtattgacattagactt
aatagcttaatttttatcaaaagaaaggaataatgtgatattaaatagtatcctcatagc
ctatattttttaaaattactatatacatgtaataaatatcagttaaatagcacaaaaaac
taacacatcattcatacaatgatcctgaaccctcaaatttgaacagaaagagttttgctt
gctcttttcttagataattactctaaattcctagtggtgttaaatatagtcctagatata
atctggcattagatttgagtaaaatcagtggagtgaagtacaactctggggaaaagcaaa
attgatattcccccccccccccggtatcttttcccttttggagtactttttaaaaatgac
attgtcccagaagaaaagtgtttattcttgtgccctttttctacagcagaactctttgaa
taaacttagaaaagtaacatgagatctcctttgcttccatcggggcgtaaaataataggg
gacattttttgtaagtgaagtagatattcatcttgattattttacatggccgataccctc
ctctagaatatgcatatgtgtagtatgttcatgtgttcaagtgggtgtacatatgtacat
gcagatgtggtctaccactgaatcgagagcttacccattcagctagagtggcagctgtcc
agtgagcagtgactgtagctagaattgccatgcctagccttttacaagatgggatccaag
cccttatcttcatcattacacatgaaatgttactgactgagtcatcaccaagctccagaa
tttgaaagtgttttagaatttaagaagtaagcacaggtgattaacaaaaggcaaagcccc
aaaagattctcttgccttcggtgttctttcaaagacttcctgaggaaaaaagatgttcgg
aaggagagagccctggcttagcatatcagctatggtgttttgtttcctgctactggggtg
ttcttcacttgggaagtgctttatgtctatgtggaagaataatggtataggcttaaccct
atagggaattatgttatttaaaactttattaccatttattacacaaaatgtacttgtata
tatatatatttttgataattggtgtgaacaatttcaagtctctttatcaacatttattta
ggttatctaagtgcttgagcactaggctcacacaagaaacctgtgataagtggcattgct
ctcaccttgtaagtaaataccaataaaatcacaggtcacccagaataaagcctcagattc
ctaatatgagattagtgagcattttctttgctcttaaaatcctgcttttcttgatttgtt
cagccactgaaatgcagtagtcctagcaagatgctcagcctgggtagagtaacctttttt
tctgtgattttttttttttttttttgaagtgtgagtatgccttccctatttccttttcca
tagttggcactctatgtgctttcattcctggaacccaaagacctgctgcaagcggctcag
acttgtcgatactggagaattttggctgaggataaccttctctggagagagaaatgtaaa
gaagagggtaagttcagaatcacacacagatgtgtttctactctggaaacaagttaacag
aataacttggctgtgagtgagagtagatggctggctcaggtgcctgccagaatgcgtctg
cgtctgttcacttccttgatcccaacaggaaaggaggccacctgatttaaaggaagccac
aaatctgcagttaaatacccacagaattgagtattgtatatactcctcgaataaacactt
actgtgcacctcctgtgagctgaacatttgtaaatgctggggacatggcactgcttcatc
ctagtctccctgttgtggaccttctattattaagtatagtttagtttggcttcaaaggga
gaatcttctgttttgtaaacaggtatctagaaaagtaatcatttagaaaaaaatagctgt
ttctgaggggcaaaatgcttgaggggggaatggtaatgctctgttctatagatcaggccc
cttggcagccgtgtgaccaggtagagagcactgacgagtgaggcggaggagggaagactc
cagggtaggatctcaggtgcttcttgctgagcctggttctacagtcaacctctggctgtt
cctagattaactgataagatcctcttcttaagttgtgcgggttttttgttttgttttatg
taaaccacggtgaatcaactctgccttaccttctttctcagaatcttttttccccttgag
acagggtgtctctatacagaccaggctggccttgaactcatgaagatccactttcctctg
cctcctgaattaaaaggatatggaattaaaagggtatgccacatacctagcttaagagtt
ctttttaaatgtcctcttaaaaaactatatttcgtaaaaatgattctgaacagcttggtt
tatatagcaagttccagggcagtccagactgtgataataacctgaggtaatggggttcat
tgtactcatagcacttaggaggcagagacaggattgctgaaagctagaggccatcttggt
ctccatagtgagttcaagccattttattgagtaggatgcagtgatattctccccattctt
ccattcctgttgcctacatgctttataagtttactgttgtaggaaactatgataagtgaa
attatacaacatttgtccttttgtgattgccttattcactgagcaaaatgtcacagcgtg
tatccccgtgtcctctgttaaggcgagcagcatttcagggtgcactgctgcatttggtgt
tcatctatggaaaggcacggtgacttgtatggacagccgttcacatctctgcttttcatc
cttttgcatatacatgcagaagtgaaagtcctgcatcctgcactgcgtttgtgtatttag
gagtcactgtgctaatttccacagcagtgcacatacagtctcatctttgcgtgttcatac
cagcacctcccacagtcactgtgagagagaatggagatagaacaaatggaggttcagtgc
tgcacagcccagtatctgggccaagcagagaaaacaagaactttataagatgtgtaattt
ttattagtggtgaaaagaattattctcttgaactctttgatactttagggattgatgaac
cgttgcacatcaagagaagaaaaataataaaaccaggtttcatacacagcccatggaaga
gtgcgtatatcagacagcacagaattgatacaaactggagacgaggagaactcaaatctc
ctaaggtaacagactcttgacatactaacagaaacctttcttcaggaacaatgcctgctc
tcttcatgtgttggctgcagtgtaactcagtgcctatcttttatggaaaggaagcagctc
tatttcaaaattaactcagagtgttaagatcagtgttcttttatttctcatttctttaat
ctgataattttattgtaatcaaggatgagttgattcaggcatggtaccacatgcttgtag
tccctgtattcaagtggccaaggcaggaggattactatgagttaaagccaacctgagcaa
tatactgagctctaggccaacgtgagctatagtatgaagccttgtctcaaaaatgtttct
ccccaaaaaggtgagttgagacagtgtgtgatactgtgctaccatgattctcactatgag
tttgcagccttctagcctcacaggccaaatggatttttttttgtgtttgtttgtttttaa
tgatttgaagcagaagtctcattttctgtcttagaatattatttcagatgtgtaaatggt
agaaatactaaatacatactttaataacaaaaagtgagccagctatgggaggcataattt
tatatgccagcatgccctgggataagttttaggttaaaaacaaaaaaccaaaaaaacaaa
aaaccattatttgctattactctgtttcttggtgctttatttttcttctctggagtgcta
aaagttggatctaaaacccactcttctcctccttttcctcttccacctctttctcttcct
tttcttctcctttactactgagctgaggttggctcagttatccaggctgccctggagctc
accttggtatcccaggcaggccttaaccctgggaccctctgttttgtcctccttgagaac
aacagtcatcaggcctggttttcttctgtttttgtcaacagtatttactaaaataaagta
acttttcttttgacttaaacagattaggcatttgtgttagaaccatctttatgaagtttt
ggcatagtaaaatgttaatagtgggcaaaggaatatggaaatatttaatgtttaatttat
catccaggaaaaagatgttgtgtcttatataaagttcattctaaaaatagattatagatt
actattctgacgcatggctccttcttgttttcactgtgtgtgtgtgtgatgtatgcacat
gtgtgtgcacgcacacacacaggtcagaggaggtagtcagttttcctgttctcattctct
gtctgacttactttttgagatagtctctctcactgatcctggagctaggctagcaattgt
aagccccagcaacacgcccctcagtgtcagggttattgtgtgaccatacacagtttttct
atgagttggagagatttggccctaggtcctgtatatctgagccatctctacagtatgtca
ctgtattctgaagaatgtgatatagtaacatccatgtgatctgtacacttttcttatagg
tgctgaaagggcatgatgaccatgtgatcacatgcctacagttttgtggcaaccgcatag
ttagtggttctgatgacaacactttaaaagtttggtcagcggtcacgggcaaggtaagtt
cactcacttctttctatatgtcagggagtcaccattttgtgtgtgtagtcacagtaaagt
gggtgtttcttaatagcaaatatcaggcctcctttagaaacacgtcccttttcctgatag
ctatgagtgtcaccctggcatgctggcagcagtagggtttccttcatttaagtagctcgt
gtctttgtcacttagtctaagatgtcccaggagcacatccttgttccttttcctcagcac
ctttcaccttgcctatgcgagttagaatcactacttcctaacgccttccaaaacatgacg
gagaaatgtgcaatttcccttatatgctgctttgttgctgagctgtctgcaatttagaaa
ccaggcacatcctgttaaggttgtcgcagacatcagtggtagggaatgaagcagggctga
agagccaagcagttgaagcagcggctgcaccgttcatacaccatggaccgttccttaacc
agtctctagtgaatattgaattatatgttcaaaagagaacattttcatgaactaagtaat
tgaccatttataaaaataacacaagaaagtatgtgaaaagcttatgtttttagaaaatat
gtcttgctgtgttgttccttggacaagagtcttacaaattctcaacttagatgttaaact
gttcagagcagctatatatttccagggtataatagtagtttgggtttatctcggagtttc
agcgtctgtctttacatgtctagaagtagcataggcagccctaactactgcctttaggca
gatggggagagctgtggtatgtggccacacccaacgctccctgcattcttgccttccttc
tcgtcctggaagacaccctacagctcagcagtattttagtcctgcggccaagctagagtt
gtggaacttgcctgctcacagtatggttgcttcctggcatgtcttatgctttgtagcata
tgattctctttaactttctttacttcattttattatatgcataagtgtttacttgtatgt
atgtctgtgcaccacatgcatatcaggtgccttccaaggccagaacagggtgtcaggtcc
ctgggcactagagttccaggtggttgggaggcaccatgtgcgtgccggagctcaaaccca
ggttcctgaaagagcaaccagtgctctaaatcactgagccatctccccagtcccttccta
tgaatcttacggtttataccatgtttcctttctccatacttgttcctcagtcttgtatcc
ctgtataagcatattctgctaccaaataagctaaaggtagtgatggtgtcagatagcatt
ggtgtgcaaaaatgctttcctagtgcttcagtaaagttgaataagtaaaatttattccag
attttaatgggatgttagtgtagtctttgacttacttgcatttctacctaaaattaatca
tctgaagtgtttttccagtgtctgagaacgttagtgggacatacaggtggagtgtggtca
tcacagatgagagacaatatcatcatcagtggatcgactgaccggactctcaaagtgtgg
aatgctgaaactggagagtgtatacatactttatatgggcacacttctactgtacggtgt
atgcatctccatgaaaaaaggtaaaggatgtgccattatgattggggctcgcattactgt
tccttgtgactctgggattcatagagtgctaatgagctcttggagtggacactaaggata
tagtgcagatggtccgaaattaagaaaattgggttatattggcttaaaagtggaggattg
tctaaatttccttcttcatttgatatatggctgtttttcttcaaactctgctctctaatc
tgttttgatgttttgatgattcatcatctttaaacaaaaatcatttcctttcttgatttt
cgtattatgctgtgtaagaacaacaaattcagtcaccaggccttgttgcctgagtcctta
agtgacacacccactctgcccccactccatcacatccctgcatatagacacagctaaggt
ccaggggccttaacttaatgtcagtactagtcatccaaactattgagagtgctcaaatat
ggggtcctcttgccttttgcacagctgacagagcgtcctcatattaggatggcttgctgc
cctacttctgcccaccaaatgaagggagaacttaaagtcccgttttcaaggtagtctcag
ccaagtctggcctgtttgccctttccttcccagttcctctgcctttatgtcaggcttctg
ctgacctcaggcccttgtgttgatacttcttcactgcacccagctaagaagttgtgtttt
ctacttctttgtcacttttggtctgacgtgtcacacacatgctgctaatctttgcactga
aaaggtagacatgggtggatactgagttcccagcttttagaggagagccttcctctgacg
ctgtagcttagaagggtccttctgtggaatccacagcattcacgacctacagctctgtcc
agactgcttttcattaagaatcattgctgtaagcagaccttccctacagccatcattgta
cagggactcctctcactcataaatttgagcctgctttgcacggtgtgttctgtgattaaa
atttagggatgggtttcaaaggacaccacatttccgttttttgttgtgttcattctccca
catgtcagcactctgctctgtctaatgtagaatggcttttttatctcttgagaatgatgt
aaaggtgcctggttagtaatctagcggtaactgcatgattgtctcgctctggttttcaga
agagtatcattttatagcactgagacagttgctttaggcatttgtgttactgctatagga
atatctttaagaatctctgagtcaccagctatgagcacgtgtctgaaactgactttccct
gactgtgaagtataaggaaacaactatggctttctgagtcccaaattcactttaaaattc
tattttgttgagaaataattgtattgaaatgtctacttactcaaatagttaataggagtt
cataattgcagtgtagtgaaatgacaaagatcataaagaaggaaagttctggattgttgt
aagttcataccacctggaggtgcattgaggatttatgcatatgccacatttccattttac
tgttaatcaggatcttgttgtgtgattggagagctgcttgcttcttgaatgcagtgcttt
ccagactgtgccatggaaccttgaggtggaaccagggtccctaagtcagggaaccaatca
gatggccagttgatctccaagtcttcctcacttcccattccaggcagaggagctggaatt
tgcatttgaaggcattaggtagagtttctcagaaaggatttctgtttaagaaaaagagga
gaaagcaatggtttacctgtcctgtgcattcatttcctgactcttgtgttctgttctgct
ctgcagggttgtaagcggttctcgagatgccactctcagggtttgggatattgagaccgg
ccagtgtttacacgtcctgatgggtcacgtagcagcggtccgctgcgttcagtatgatgg
caggagggttgttagtggagcttatgattttatggtgaaggtgtgggatccagagactga
gacctgtctacacacgttacagggacacactaatagagtctattcattacaggtaagagg
ccgtgtctctcccaccctttgattctctgccgttgactcataagattgttctcctcagtt
actgtcaagttgctgtttataagtaccttaaaattagcatgaagaaacacaaatttcaaa
tcatttaactgatttaatgatttaaatttctgtgatccattttctctttaaaatacaaat
atttgtcttggaacctgatacttcaggattataaattttgctacactaattaatacttag
ataatctcataggagaataacttggattttaataaactccttagctgaggattaatttga
ccaaaagctgcccctttgccttgatagctaaaagttgatggagcatgtaattgtgatttc
atccccatttgaagtagctgtggaacctctctgcagaaagctctgtacttaacttgccca
gagccactgactgggagccacagagagatgaatttgactaacctgttgttttcctttaga
tttgaacttgatatttattaatagactgtaatagttcaagatcttcagtaacaagacata
aaacgtgggatacttttatttctggaaatgcagcattttccatcttaaaatatttcctgt
tacataatttccttcccagttactactgcctgttagtgatgagcaaagtagaacagaatc
ccagtgctgatgttgccgttcttagggcatttgattcttaattgatgatactgcccaagt
aatgaagtattgatatataataaagatgttgattgcttcagaaaagaggatggcttccaa
ggggcgggaattggttgctaatgctgctgcttgtgatgtgctcagaactgtagagaatga
tctcagaatagatagctgcatgtcatagagaccttctggggactggtagatgcttattgg
cttttagtgtccttgggaagctgaagaagagagactacatagattgcgtttgtttctaaa
ataccccctcttctaattcctgatacagttttcttttgggatatttgctaattagcatta
atttcttgtaaacaacttcattttgcaaacactttttaatggaatttactattgaacatc
atgaaagttatctgtggctattcccttgcctcaggtgctgataaacatgtctctgtctca
ggcagtggctttggcttcctctagacaagtcaggctccatagtcctactaaatacctggg
atgtagcttccagctgggaccaggtggtctgaaaaaaatgttgtttatttcaggtgcttt
gttgccttcttgaagttgtttctgccttgtttgaacgattcggctgttttgttttgtttc
tttttttaacttttaagagaatttatgaaagaagtattttgaagacatgatctatagact
ttcatggcatgcacatcagcttacctgcaagtttatcccagtaggctctttgttctggcc
tacgtgttgcttgtaccctctccctattgtgagtaatgtcaacagctgataggacaggaa
tccgtgtttctgttctaatactctgtgtctgttgtagtttgatggcatccatgtggtgag
tggatctcttgatacatcaatccgagtctgggatgtggagacagggaattgtattcacac
gctaacaggacaccagtcattaacgagtggaatggaactcaaagacaatattcttgtctc
tgggaatgcagattctacagttaagatctgggatatcaaaacaggacagtgtttacaaac
tttgcaaggtaagtcctggccctctgttagcctatctttaaaaacatcattgctattcaa
gagagaacctgtggagaacagaggtttaaccttatgcagagtacttactcctgatgagtt
tgtgaatgtttggctttcaaaagtaagccaccttgagaaatggttttcaattgtgtcttt
aatgtgaaaattcttaatcatttcctgtgtgttctgttgactctgtttaacatggctgtc
tcatagggcagcgttctttttgagtgatcattttattttgaattgaagcatctctgatag
gaaatgactttggccttgcttggtcaattccttcagtcggaatggaaggagtaatggtgc
tctctgaggggaaggtagagtgagaagcattagcattaggtatagcaccattggcttctc
catgagtaacttccttctcttagtcacagcccttagaaggtgttgttgttgtttgtttgt
ttgtttgtttggagtgaagccatcaggaggcagcacatagcagcctttgttcattaattg
tgactgttcctaggagtttggggtagtcacaaattctatttccttctttgactgatcagt
tcagtgtggcagagtgacagagtgacaaacctgcacgttcccttttccagcagtggaatg
tgtacactggctgaccagggccactgggagccggagctcttgagacttggtactgtagtt
acatagccaccagatttccttacatctaagctggtcctaaactacagcaaggttgctaac
tgtgcagctttttaaatactgcatttcttgtatgccataatgtagaagagcacaagtccc
acgcagaagccctgaacgtccttcctaagcagtaacccgcatttgcctttgcctaggtcc
cagcaagcatcagagcgctgtgacctgcttacagttcaacaagaacttcgtaattaccag
ctcagacgacggaacggtcaaactctgggacttgaaaacgggtgaatttatccgaaacct
cgtcacattggagagtggggggagcgggggagttgtgtggcggatcagggcctcaaacac
aaagctggtgtgtgcagtcgggagtcggaatggaactgaggaaaccaagctcctggtgct
ggactttgatgtggacatgaaatgaaaagcagacatgatgaattttgtccaactgtgtag
acaatatactccctacccttccccctgcgcaaaaaacaaaaacaaacaaacaaaaaaatg
aaaaaaaaaaacagaaaaaaaaaaagagaaaaaagaaaaggaaaaaaatcccttgtactc
agtggtgcaggatgttggcttgggacaacagactgaaaagacctacagactaagaaggca
agaagagacaagagaccgtaactgacaggaggcggcagctgtcgcatctcgcaaaggcct
cacttgtgactgaggggcagcttggcaagacgactctctaaatccaaccaggtgcaatta
ttctttgttttcttctccagtggtcattgagcagagctacatcagcgttgttaccgtcac
ctagaaaggagtggcagtaatatccaaacacgggctgcttatcttctaatcagagcatct
gcaacaaaccgtcatttttctgaagtggaaaagcttaaaacaattactgtgaattgtttt
tgtacagttatcatgaagctttcttttttctctttttcctgtttctcttcttttcttttt
tcttttttttttttgccaaccattgccaatgtcaatcaatcacagtattagcctctgtta
atctatctctttactgttgcttctactcttcaatgcatatgttgctcaaaggtggcaagt
tgtcctgggttgtgtgagtcctgagatggatataattcttgatgctggtgctagaagtag
gtcttcaaatccggggtcgttgtcccacccctgtactgtactcccagtggccaaacttat
ttatgctgctaaatgaaagaaagaaaaagcaaattattttttttttattttttttctgct
gtgacgttttagtcccagactgaattccaaatttgctctagtttggttacagaaaaaaaa
gacttttttgccactgaaacttgagccatctgtgcctctaagaggctgagaatggaaaag
tttcagataataaagagtgaagtttgcctgcaaataaagaattgagagtgtgtgcaaagc
ttattttcttttatctgggcaaaaattaaaacacattccttgggacagagcctgaggtgc
ctgttctgtggagaaacttctttttgagggctgtggtgaatggaagaacatacatagcaa
aactgacaagatattttaaagatatataaaacacaaaggaaaaggaggttgctggtcagt
cgtagcatcttacagtattggggaaaacaactgttacagtttcattgctctgagtgactg
acgtgagaggaattcgctctgcagtgacgctgtctgtcactcgcctaccagctcgacgag
caagagagcgggagtcagatggtccgcctcattcaccaggagccgtaactcaagctgaac
tgtgaaagtggttaacactgtatcctaggccgtcttttttttcctcctcctgtttatttt
ttgtttgttttatttatagtctgatttaaaacaatcagattcaagttggttaattttagt
tatgtaacaacctgacgtgatggaggaaacaacctgtaaagggattgtgtctatggtttg
attcacttagaaattttattttcttataacttaagtgcaataaaatgtgttttttcatgt
ta
PS

TABLE 3
NGN3 protein sequence

| | | |
|---|---|---|
| NGN3 protein | Human (Homo | MTPQPSGAPTVQVTRETERSFPRASEDEVTCPTSAPPSPTRTRGNCA |
| sequence | sapiens) | EAEEGGCRGAPRKLRARRGGRSRPKSELALSKQRRSRRKKANDRERN |
| | NCBI Reference | RMHNLNSALDALRGVLPTFPDDAKLTKIETLRFAHNYIWALTQTLRI |
| | Sequence: | ADHSLYALEPPAPHCGELGSPGGSPGDWGSLYSPVSQAGSLSPAASL |
| | NP_066279.2 | EERPGLLGATFSACLSPGSLAFSDFL |
| | | (SEQ ID NO: 5) |
| | Mouse (Mus | MAPHPLDALTIQVSPETQQPFPGASDHEVLSSNSTPPSPTLIPRDCS |
| | musculus) | EAEVGDCRGTSRKLRARRGGRNRPKSELALSKQRRSRRKKANDRERN |
| | NCBI Reference | RMHNLNSALDALRGVLPTFPDDAKLTKIETLRFAHNYIWALTQTLRI |
| | Sequence: | ADHSFYGPEPPVPCGELGSPGGGSNGDWGSIYSPVSQAGNLSPTASL |
| | NP_033849.3 | EEFPGLQVPSSPSYLLPGALVFSDFL |
| | | (SEQ ID NO: 6) |

TABLE 4
NGN3 nucleotide sequence

| | | |
|---|---|---|
| NGN3 | Human (Homo | gataaagcgtgccaaggggcacacgacttgctgctcaggaaatccct |
| nucleotide | sapiens) | gcggtctcaccgccgcgcctcgagagagagcgtgacagaggcctcgg |
| sequence | NCBI Reference | accccattctctcttcttttctcctttggggctggggcaactcccag |
| | Sequence: | gcgggggcgcctgcagctcagctgaacttggcgaccagaagcccgct |
| | NC_000010.10 | gagctccccacggccctcgctgctcatcgctctctattcttttgcgc |
| | | cggtagaaaggtaatatttggaggcctccgagggacgggcaggggaa |
| | | agagggatcctctgacccagcgggggctgggaggatggctgtttttg |
| | | ttttttcccacctagcctcggaatcgcggactgcgcccagtgacgga |
| | | ctcaaacttacccttccctctgaccccgccgtaggatgacgcctcaa |
| | | ccctcgggtgcgcccactgtccaagtgacccgtgagacggagcggtc |
| | | cttccccagagcctcggaagacgaagtgacctgccccacgtccgccc |
| | | cgcccagccccactcgcacacgggggaactgcgcagaggcggaagag |
| | | ggaggctgccgaggggccccgaggaagctccgggcacggcgcggggg |
| | | acgcagccggcctaagagcgagttggcactgagcaagcagcgacgga |
| | | gtcggcgaaagaaggccaacgaccgcgagcgcaatcgaatgcacaac |
| | | ctcaactcggcactggacgccctgcgcggtgtcctgcccaccttccc |
| | | agacgacgcgaagctcaccaagatcgagacgctgcgcttcgcccaca |
| | | actacatctgggcgctgactcaaacgctgcgcatagcggaccacagc |
| | | ttgtacgcgctggagccgccggcgccgcactgcggggagctgggcag |
| | | cccaggcggttcccccggggactgggggtccctctactccccagtct |
| | | cccaggctggcagcctgagtcccgccgcgtcgctggaggagcgaccc |
| | | gggctgctgggggccaccttttccgcctgcttgagcccaggcagtct |
| | | ggctttctcagattttctgtgaaaggacctgtctgtcgctgggctgt |
| | | gggtgctaagggtaagggagagggagggagccgggagccgtagaggg |
| | | tggccgacggcggcggccctcaaaagcacttgttccttctgcttctc |
| | | cctggctgacccctggccggcccaggcctccacgggggcggcaggct |
| | | gggttcattccccggccctccgagccgcgccaacgcacgcaaccctt |
| | | gctgctgcccgcgcgaagtgggcattgcaaagtgcgctcattttagg |
| | | cctcctctctgccaccaccccataatctcattcaaagaatactagaa |
| | | tggtagcactacccggccggagccgcccaccgtcttgggtcgcccta |
| | | ccctcactca |
| | | (SEQ ID NO: 7) |
| | Mouse (Mus | atgcagctcagaaatccctctgggtctcatcactgcagcagtggtcg |
| | musculus) | agtacctcctcggagcttttctacgacttccagacgcaatttactcc |
| | NCBI_Reference | aggcgagggcgcctgcagtttagcagaacttcagagggagcagagag |
| | Sequence: | gctcagctatccactgctgcttgacactgaccctatccactgctgct |
| | NC_000076.6 | tgtcactgactgacctgctgctctctattcttttgagtcgggagaac |
| | | taggtaacaattcggaaactccaaagggtggatgaggggcgcgcggg |
| | | gtgtgtgtgggggatactctggtcccccgtgcagtgacctctaagtc |
| | | agaggctggcacacacacaccttccattttttcccaaccgcaggatg |
| | | gcgcctcatcccttggatgcgctcaccatccaagtgtccccagagac |
| | | acaacaaccttttcccggagcctcggaccacgaagtgctcagttcca |
| | | attccaccccacctagccccactctcatacctagggactgctccgaa |
| | | gcagaagtgggtgactgccgagggacctcgaggaagctccgcgcccg |
| | | acgcggagggcgcaacaggcccaagagcgagttggcactcagcaaac |
| | | agcgaagaagccggcgcaagaaggccaatgatcgggagcgcaatcgc |
| | | atgcacaacctcaactcggcgctggatgcgctgcgcggtgtcctgcc |
| | | caccttcccggatgacgccaaacttacaaagatcgagaccctgcgct |
| | | tcgcccacaactacatctgggcactgactcagacgctgcgcatagcg |
| | | gaccacagcttctatggcccggagccccctgtgccctgtggagagct |
| | | ggggagccccggaggtggctccaacggggactggggctctatctact |
| | | ccccagtctcccaagcgggtaacctgagccccacggcctcattggag |
| | | gaattccctggcctgcaggtgcccagctccccatcctatctgctccc |
| | | gggagcactggtgttctcagacttcttgtgaagagacctgtctggct |
| | | ctgggtggtgggtgctagtggaaagggaggggaccagagccgtctg |
| | | gagtgggaggtagtggaggctctcaagcatctcgcctcttctggctt |
| | | tcactacttggatccctagccctctcacagggcttaactaggcttct |
| | | catcggtacccttgctgctgcgcacagcagacattgggggctgctct |
| | | tctcttaactctcctcggtgcagccacatcaaactctcgctccaagc |
| | | atttgagaatggtagcactacctagttggagactcccatacttcctg |
| | | gtgagtctgccctcattcaaatctgccggcctccgaccatccatcac |
| | | tttttccagggtgacctaatccagtgttgcgtcttacctcactggct |
| | | cctccatccagctcttggcccatagatgatgttcgtcgtctttactg |
| | | cccgctacatgcagggtttctgagcttctccattctgccttagtcca |
| | | cgaaggtgatctgccttcttctgcacttttcaagtcgttacccttcc |
| | | cccaagggagaccaggctgtgaaccggaaagccctagcctatggcta |
| | | gagcatctctccaacttgtctcccgtgtctaaagtgtgagttgcagg |
| | | gacggttcctgaagcactgtttgtctccct |
| | | (SEQ ID NO: 8) |

### REFERENCES

1 Mathis, D., Vence, L. & Benoist, C. beta-Cell death during progression to diabetes. Nature 414, 792-798, (2001).
2 Bonner-Weir, S. & Sharma, A. Are there pancreatic progenitor cells from which new islets form after birth? Nature clinical practice. Endocrinology & metabolism 2, 240-241, (2006).
3 Welcker, M. & Clurman, B. E. FBW7 ubiquitin ligase: a tumour suppressor at the crossroads of cell division, growth and differentiation. Nat Rev Cancer 8, 83-93 (2008).
4 Wang, Z. et al. Emerging roles of the FBW7 tumour suppressor in stem cell differentiation. Nature Publishing Group 13, 36-43, (2012).
5 Edlund, H. Pancreatic organogenesis—developmental mechanisms and implications for therapy. Nature reviews Genetics 3, 524-532, (2002).
6 Xu, X. et al. Beta cells can be generated from endogenous progenitors in injured adult mouse pancreas. Cell 132, 197-207, (2008).
7 Bonner-Weir, S. et al. The pancreatic ductal epithelium serves as a potential pool of progenitor cells. Pediatric diabetes 5 Suppl 2, 16-22, (2004).
8 Zhou, Q., Brown, J., Kanarek, A., Rajagopal, J. & Melton, D. A. In vivo reprogramming of adult pancreatic exocrine cells to beta-cells. Nature 455, 627-632, (2008).
9 Thorel, F. et al. Conversion of adult pancreatic α-cells to β-cells after extreme β-cell loss. Nature 464, 1149-1154, (2010).
10 Collombat, P. et al. The ectopic expression of Pax4 in the mouse pancreas converts progenitor cells into alpha and subsequently beta cells. Cell 138, 449-462, (2009).
11 Piper, K. et al. Beta cell differentiation during early human pancreas development. The Journal of endocrinology 181, 11-23 (2004).
12 Oliver-Krasinski, J. M. & Stoffers, D. A. On the origin of the beta cell. Genes &amp; development 22, 1998-2021, (2008).
13 Sancho, R. et al. F-box and WD repeat domain-containing 7 regulates intestinal cell lineage commitment and is a haploinsufficient tumor suppressor. Gastroenterology 139, 929-941, (2010).
14 Hoeck, J. D. et al. Fbw7 controls neural stem cell differentiation and progenitor apoptosis via Notch and c-Jun. Nat Neurosci 13, 1365-1372, (2010).
15 Ishikawa, Y., Onoyama, I., Nakayama, K. I. & Nakayama, K. Notch-dependent cell cycle arrest and apoptosis in mouse embryonic fibroblasts lacking Fbxw7. Oncogene 27, 6164-6174, (2008).
16 Thompson, B. J. et al. Control of hematopoietic stem cell quiescence by the E3 ubiquitin ligase Fbw7. J Exp Med 205, 1395-1408 (2008).
17 Means, A. L., Xu, Y., Ray, K. C. & Gu, G. A CK19(CreERT) knockin mouse line allows for conditional DNA recombination in epithelial cells in multiple endodermal organs. Genesis (New York, N.Y.: 2000) 46, 318-323, (2008).
18 Teta, M., Long, S. Y., Wartschow, L. M., Rankin, M. M. & Kushner, J. A. Very slow turnover of beta-cells in aged adult mice. Diabetes 54, 2557-2567 (2005).
19 Graf, T. & Enver, T. Forcing cells to change lineages. Nature 462, 587-594, (2009).
20 Mikkola, I., Heavey, B., Horcher, M. & Busslinger, M. Reversion of B cell commitment upon loss of Pax5 expression. Science 297, 110-113, (2002).
21 Hingorani, S. R. et al. Preinvasive and invasive ductal pancreatic cancer and its early detection in the mouse. Cancer cell 4, 437-450 (2003).
22 Srinivas, S. et al. Cre reporter strains produced by targeted insertion of EYFP and ECFP into the ROSA26 locus. BMC Dev Biol 1, 4 (2001).
23 Ventura, A. et al. Restoration of p53 function leads to tumour regression in vivo. Nature 445, 661-665, (2007).
24 Jandke, A. et al. The F-box protein Fbw7 is required for cerebellar development. Developmental biology 358, 201-212, (2011).
25 Nateri, A. S., Spencer-Dene, B. & Behrens, A. Interaction of phosphorylated c-Jun with TCF4 regulates intestinal cancer development. Nature 437, 281-285 (2005).
26 Crusio, K. M., King B., Reavie L. B. & Afiantis I. The ubiquitous nature of cancer: the role of the SCF (Fbw7) complex in development and transformation. Oncogene 29(35), 4865-73 (2010).
27 Perry J. M. & Li L. Self-renewal versus transformation: Fbxw7 deletion leads to stem cell activation and leukemogenesis. Genes & Development 22(9): 1107-9, 2008.
28 Grapin-Botton A. Ductal cells of the pancreas. International Journal of Biochemistry and Cell Biology 37(3): 504-10, 2005.
29 Rukstalis J. M. & Habener J. F. Neurogenin3: a master regulator of pancreatic islet differentiation and regeneration. Islets 1(3): 177-184, 2009.

1. A method for reprogramming a somatic cell to a pancreatic β-cell, the method comprising the step of decreasing expression or activity of Fbw7 in the somatic cell.

2. The method according to claim 1, wherein the somatic cell is a pancreatic ductal cell, a skin fibroblast or a keratinocyte.

3. The method according to claim 1 or 2, wherein the method is carried out ex vivo.

4. The method according to claim 1 or 2, wherein the method is carried out in vivo.

5. The method according to claim 4, wherein the method is carried out in a subject with a metabolic disorder, such as diabetes, or in a subject at risk of developing a metabolic disorder, such as diabetes.

6. The method according to any one of the preceding claims, wherein the method comprises contacting the somatic cell with an agent that reduces the expression or activity of Fbw7 in the somatic cell.

7. The method according to claim 6, wherein the activity of Fbw7 is phosphorylation-mediated degradation of NGN3.

8. The method according to claim 7, wherein the agent that reduces the expression or activity of Fbw7 inhibits phosphorylation of NGN3 by GSK3β.

9. The method according to claim 8, wherein the agent is an inhibitor of GSK3β, for example lithium chloride, CHIR-99021 (CT99021), SB 216763, TWS119, CHIR-98014, SB 415286, tideglusib, LY2090314 or TDZD-8.

10. The method according to any one of claims 6 to 8, wherein the agent that reduces the expression or activity of Fbw7 is a small molecule, a nucleotide sequence, a protein, a peptide, a modified peptide, an aptamer or an RNA interference (RNAi) agent.

11. An agent that reduces the expression or activity of Fbw7 for use in a method of reprogramming a somatic cell to a pancreatic β-cell, the method comprising the step of contacting the somatic cell with said agent.

12. An agent for use according to claim 11, wherein the somatic cell is a pancreatic ductal cell, a skin fibroblast or a keratinocyte.

13. An agent for use according to claim 11 or 12, wherein the activity of Fbw7 is phosphorylation-mediated degradation of NGN3.

14. An agent for use according to claim 13, wherein the agent inhibits phosphorylation of NGN3 by GSK3β.

15. A screening method for identifying a candidate agent for reprogramming a somatic cell to a pancreatic β-cell, the method comprising determining the ability of the agent to reduce expression or activity of Fbw7, wherein an agent that reduces expression or activity of Fbw7 is likely to be able to reprogram a somatic cell to a pancreatic β-cell.

16. The method according to claim 15, wherein the somatic cell is a pancreatic ductal cell, a skin fibroblast or a keratinocyte.

17. The method according to claim 15 or 16, wherein the activity of Fbw7 is phosphorylation-mediated degradation of NGN3.

18. The method according to claim 17, wherein the phosphorylation-mediated degradation of NGN3 is phosphorylation of NGN3 by GSK3β.

19. A method for reprogramming a somatic cell to a pancreatic β-cell, the method comprising the step of decreasing phosphorylation-mediated degradation of NGN3 in the somatic cell.

20. The method according to claim 19, wherein the somatic cell is a pancreatic ductal cell, a skin fibroblast or a keratinocyte.

21. The method according to claim 19 or 20, wherein the method comprises the step of decreasing phosphorylation of NGN3 by GSK3β.

22. The method according to any one of claims 19-21, wherein the method is carried out ex vivo.

23. The method according to any one of claims 19-22, wherein the method is carried out in vivo.

24. The method according to any of claims 19-23, wherein the method comprises the step of contacting the somatic cell with an agent that decreases the phosphorylation of NGN3 by GSK3β.

25. The method according to claim 24, wherein the agent is an inhibitor of GSK3β, for example lithium chloride, CHIR-99021 (CT99021), SB 216763, TWS119, CHIR-98014, SB 415286, tideglusib, LY2090314 or TDZD-8.

26. An agent that decreases the phosphorylation-mediated degradation of NGN3 for use in a method of reprogramming a somatic cell to a pancreatic β-cell, the method comprising the step of contacting the somatic cell with said agent.

27. An agent for use according to claim 26, wherein the somatic cell is a pancreatic ductal cell, a skin fibroblast or a keratinocyte.

28. An agent for use according to claim 26 or 27, wherein the agent inhibits phosphorylation of NGN3 by GSK3β.

29. An agent for use according to claim 28, wherein the agent is an inhibitor of GSK3β kinase activity, such as lithium chloride, CHIR-99021 (CT99021), SB 216763, TWS119, CHIR-98014, SB 415286, tideglusib, LY2090314 or TDZD-8.

30. A screening method for identifying a candidate agent for reprogramming a somatic cell to a pancreatic β-cell, the method comprising determining the ability of the agent to decrease phosphorylation-mediated degradation of NGN3, wherein an agent that decreases phosphorylation-mediated degradation of NGN3 is likely to be able to reprogram a somatic cell to a pancreatic β-cell.

31. The method according to claim 30, wherein the somatic cell is a pancreatic ductal cell, a skin fibroblast or a keratinocyte.

32. The method according to claim 30 or 31, wherein the method comprises the step of determining the ability of the agent to decrease phosphorylation of NGN3 by GSK3β, wherein an agent that decreases phosphorylation of NGN3 by GSK3β is likely to be able to reprogram a somatic cell to a pancreatic β-cell.

33. A method of treating a subject with a metabolic disorder or a subject at risk of developing a metabolic disorder, the method comprising the step of administering to the subject pancreatic β-cells obtained by the method of any one of claim 1-10 or 19-25.

34. A method according to claim 33, wherein the method includes:
(i) the step of reprogramming a somatic cell to a pancreatic β-cell using the method of any one of claim 1-10 or 19-25; and
(ii) the step of administering the β-cells obtained in step (i) to the subject.

35. A method according to claim 33 or 34, wherein the metabolic disorder is diabetes.

36. A pancreatic β-cell obtained by the method of any one of claim 1-10 or 19-25 for use in a method for treatment of a subject with a metabolic disorder or a subject at risk of developing a metabolic disorder, said method comprising administering said pancreatic β-cell to the subject.

37. A pancreatic β-cell for use according to claim 36, wherein the metabolic disorder is diabetes.
